(19) United States
(12) Patent Application Publication
(10) Pub. No.: US 2025/0171432 A1
(43) Pub. Date: May 29, 2025

(54) COMPOUNDS AND METHODS OF USE

(71) Applicant: Tango Therapeutics, Inc., Boston, MA (US)

(72) Inventors: Kevin M. COTTRELL, Boston, MA (US); John P. MAXWELL, Boston, MA (US)

(21) Appl. No.: 18/833,486

(22) PCT Filed: Jan. 26, 2023
(86) PCT No.: PCT/US2023/011658

§ 371 Date: Jul. 26, 2024

Related U.S. Application Data
(60) Provisional application No. 63/303,497, filed on Jan. 26, 2022.

(51) Int. Cl. C07D 417/14; A61K 31/4545; A61K 31/4709; A61K 31/496; A61K 31/5377; A61K 31/55; C07D 401/12; C07D 401/14; C07D 405/14; C07D 413/14; C07D 471/04; C07D 471/08; C07D 487/08; C07D 491/107; C07D 491/113

(52) U.S. Cl. CPC C07D 417/14; A61K 31/4545; A61K 31/4709; A61K 31/496; A61K 31/5377; A61K 31/55; C07D 401/12; C07D 401/14; C07D 405/14; C07D 413/14; C07D 471/04; C07D 471/08; C07D 487/08; C07D 491/107; C07D 491/113

(57) ABSTRACT

Provided are compounds of Formula (I): (I), and pharmaceutically acceptable salts thereof, and pharmaceutical compositions, processes of preparing and methods of treating thereof; wherein R¹, R², R³, R⁴, R⁶, R⁷, R⁸ and n are as defined herein and wherein the compounds are selected from the group consisting of the compounds of Table 1.

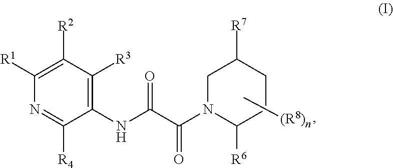

### CROSS-REFERENCE TO RELATED APPLICATION

This application claims priority to U.S. Provisional Application No. 63/303,497, filed on Jan. 26, 2022, which is incorporated by reference herein in its entirety and for all purposes.

### FIELD

Provided herein are compounds, and compositions and methods thereof. In some embodiments, provided are compounds for inhibiting protein arginine methyltransferase 5 (PRMT5). In some embodiments, provided are methods for treatment of diseases or disorders, such as cancer.

### BACKGROUND

Protein arginine methyltransferase 5 (PRMT5) is a type II arginine methyltransferase that regulates essential cellular functions, including the regulation of cell cycle progression, apoptosis and the DNA-damage response (Koh, C. et al., Curr Mol Bio Rep 2015; Wu et al., Nat Rev Drug Discovery 2021). MTAP is a critical enzyme in the methionine salvage pathway, a six-step process that recycles methionine from the product of polyamine synthesis, methylthioadenosine (MTA). Loss of MTAP causes the accumulation of its substrate, MTA, which has been described to function as a SAM-competitive PRMT5 inhibitor (Kruykov et al., 2016; Marjon et al., 2016 and Markarov et al., 2016). Data from genome-wide genetic perturbation screens using shRNA suggests a selective requirement for PRMT5 activity particularly inMTAP-deleted cancer cell lines (Kruykov et al., 2016; Marjon et al., 2016 and Markarov et al., 2016). It is proposed that the accumulation of MTA caused by MTAP-deletion in these cell lines partially inhibits PRMT5, rendering those cells selectively sensitive to additional PRMT5 inhibition.

A PRMT5 inhibitor that leverages the accumulation of MTA by binding in an MTA-uncompetitive, non-competitive or mixed mode manner or in a MTA-cooperative binding manner may demonstrate selectivity for MTAP-deleted tumor cells. Some PRMT5 inhibitors are currently being explored for therapeutic uses (e.g., for treating cancer), however there are currently no such PRMT5 therapies approved by the United States Food and Drug Administration that demonstrate selectivity for MTAP-deleted cancer cell lines.

Accordingly, there is a need for PRMT5 inhibitors for treating diseases, such as cancers.

### SUMMARY

In one aspect, provided is a compound selected from the compounds of Table 1 or a pharmaceutically acceptable salt thereof.

In one embodiment, provided is a pharmaceutical composition comprising a compound from the compounds of Table 1, or a pharmaceutically acceptable salt thereof, as defined herein and a pharmaceutically acceptable carrier. In some embodiments, the pharmaceutical composition further comprises a second therapeutic agent.

In one embodiment, provided is a method of treating an MTAP-deficient and/or an MTA-accumulating disease in a subject in need thereof by administering to the subject an effective amount (e.g., a therapeutically effective amount) of compound selected from the compounds of Table 1, or a pharmaceutically acceptable salt thereof, as defined herein or a pharmaceutically acceptable composition thereof. In some embodiments, the compound or composition is administered in combination with a second therapeutic agent.

In one embodiment, provided is a method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering an effective amount (e.g., a therapeutically effective amount) of a compound of selected from the compounds of Table 1 or a pharmaceutical composition thereof to the subject identified in step b).

### DETAILED DESCRIPTION

The disclosure herein sets forth exemplary methods, parameters and the like. It should be recognized, however, that such description is not intended as a limitation on the scope of the present disclosure but is instead provided as a description of exemplary embodiments.

As generally described herein, provided are compounds (e.g., compounds of Table 1, or pharmaceutically acceptable salts thereof) that are MTA-uncompetitive PRMT5 inhibitors useful for treating proliferating disorders (e.g., cancers) associated with MTAP deficiencies and/or MTA accumulation.

In some embodiments, provided are compounds (e.g., compounds of Table 1, or pharmaceutically acceptable salts thereof) that are MTA-uncompetitive, non-competitive or mixed mode PRMT5 inhibitor or an MTA cooperative binding agent useful for treating proliferating disorders (e.g., cancers) associated with MTAP deficiencies and/or MTA accumulation.

### Definitions

As used in the present disclosure, the following words and phrases are generally intended to have the meanings as set forth below unless expressly indicated otherwise or the context in which they are used indicates otherwise.

### MTAP

“MTAP” as used herein refers to methylthioadenosine phosphorylase, an enzyme in the methionine salvage pathway, also known as S-methyl-5′-thioadenosine phosphorylase; also known as BDMF; DMSFH; DMSMFH; LGMBF; MSAP; and c86fus. External IDs: OMIM: 156540 MGI: 1914152 HomoloGene:1838 chEMBL: 4941 GeneCards: MTAP Gene; Entrez 4507; RefSeq (mRNA): NM_002451; location: Chr 9: 21.8-21.93 Mb. By “wild-type” MTAP is meant that encoded by NM_002451 or having the same amino acid sequence (NP_002442). (Schmid et al. Oncogene 2000, 19, pp 5747-54). As used herein, the term “MTAP-deficient”, “MTAP-deficiency”, “MTAP-null” and the like refer to cells (including, but not limited to, cancer cells, cell lines, tissues, tissue types, tumors, etc.) that have a significant reduction in post-translational modification, production, expression, level, stability and/or activity of MTAP relative to that in a control, e.g., reference or normal or non-cancerous cells. The reduction can be at least about 20%, 30%, 40%, 50%, 60%, 70%, 80% or 90%. In some embodiments, the reduction is at least 20%. In some embodiments, the reduction is at least 50%. The terms “MTAP-deficient and/or MTA accumulating”, “MTAP-deficient and/or MTA-accumulating”, MTAP deficient and/or MTA upregulated” and the like, regarding a cell or cells, etc., indicate that the cell or cells, etc., either are deficient in MTAP and/or overproduce or accumulate MTA. MTAP-deficient cells include those wherein the MTAP gene has been mutated, deleted, or transcriptionally silenced. As a non-limiting example, MTAP-deficient cells can have a homozygous deletion. MTAP knockdown is not lethal. In some embodiments, the MTAP-deficient cells are also CDKN2A-deficient. The MTAP deficiency can be detected using any reagent or technique known in the art, for example: immunohistochemistry utilizing an antibody to MTAP, and/or genomic sequencing, and/or nucleic acid hybridization and/or amplification utilizing at least one probe or primer comprising a sequence of at least 12 contiguous nucleotides (nt) of the sequence of MTAP, wherein the primer is no longer than about 30 nt.

An “MTAP-deficiency-related” or “MTAP-deficiency” or “MTAP deficient” disease (for example, a proliferating disease, e.g., a cancer) or a disease (for example, a proliferating disease, e.g., a cancer) “associated with MTAP deficiency” or a disease (for example, a proliferating disease, e.g., a cancer) “characterized by MTAP deficiency” and the like refer to an ailment (for example, a proliferating disease, e.g., a cancer) wherein a significant number of cells are MTAP-deficient. For example, in a MTAP-deficiency-related disease, one or more disease cells can have a significantly reduced post-translational modification, production, expression, level, stability and/or activity of MTAP. Examples of MTAP-deficiency-related diseases include, but are not limited to, cancers, including but not limited to: glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma (See FIG. 1). In a patient afflicted with a MTAP-deficiency-related disease, it is possible that some disease cells (e.g., cancer cells) can be MTAP-deficient while others are not. Similarly, some disease cells may be MTA-accumulating while others are not. Thus, the present disclosure encompasses methods of treatment involving diseases of these tissues, or any other tissues, wherein the proliferation of MTAP-deficient and/or MTA-accumulating cells can be inhibited by administration of a PRMT5 inhibitor.

Some cancer cells which are MTAP-deficient are also deficient in CDKN2A; the post-translational modification, production, expression, level, stability and/or activity of the CDKN2A gene or its product are decreased in these cells. The genes for MTAP and CDKN2A are in close proximity on chromosome 9p21; MTAP is located approximately 100 kb telomeric to CDKN2A. Many cancer cell types harbor CDKN2A/MTAP loss (loss of both genes). Thus, in some embodiments, a MTAP-deficient cell is also deficient in CDKN2A.

### MTA and MTA Accumulation

By “MTA” is meant the PRMT5 inhibitor also known as methyl-thioadenosine, S-methyl-5′-thioadenosine, [5′deoxy-5′-(methylthio)-fl-D-ribofuranosyl]adenine, 5′-methyl-thioadenosine, 5′-deoxy, 5′-methyl thioadenosine, and the like. MTA selectively inhibits PRMT5 methyltransferase activity. MTA is the sole known catabolic substrate for MTAP. The terms “MTA accumulating”, “MTA overproducing”, “MTA upregulated” and the like refer to cells (including, but not limited to, cancer cells, cell lines, tissues, tissue types, tumors, etc.) that have a significantly increased production, level and/or stability of MTA. MTA-accumulating cells include those wherein the cells comprise at least about 20%, 30%, 40%, 50%, 60%, 70%, 80%, 90%, 100%, or greater than 100%, higher production, level and/or stability of MTA than that in normal or non-cancerous cells. In some embodiments, MTA-accumulating cells include those wherein the cells comprise at least 20% higher production, level and/or stability of MTA than that in normal or non-cancerous cells. In some embodiments, MTA-accumulating cells include those wherein the cells comprise at least 50% higher production, level and/or stability of MTA than that in normal or non-cancerous cells. Determination of MTA accumulation in test samples (e.g., cells such as cancer cells being tested for MTA accumulation) and reference samples, and other cells, tissues, samples, etc., can be performed using any method known in the art. Such methods for detecting MTA include, as a non-limiting example, liquid chromatography-electrospray ionization-tandem mass spectrometry (LC-ESI-MS/MS), as described in Stevens et al. J. Chromatogr. A. 2010, 1217, pp 3282-3288; and Kirovski et al. Am. J. Pathol. 2011, 178, pp 1145-1152; and references cited therein. Loss of MTAP is associated with accumulation of MTA (Williams-Ashman et al. Biochem. Pharm. 1982, 31, pp 277-288; and Limm et al. Eur. J. Cancer. 2013, 49, Issue 6.

An “MTA-accumulation-related”, “MTA-accumulation”, “MTA-accumulating”, “MTA overproducing”, “MTA upregulated” disease (for example, a proliferating disease, e.g., a cancer) or a disease (for example, a proliferating disease, e.g., a cancer) “associated with MTA accumulation” or a disease (for example, a proliferating disease, e.g., a cancer) “characterized by MTA accumulation” and the like refer to an ailment (for example, a proliferating disease, e.g., a cancer) wherein a significant number of cells are MTA accumulating. Examples of MTA-accumulating diseases include, but are not limited to, cancers, including but not limited to: glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma (See FIG. 1). In a patient afflicted with a MTAP-deficiency-related disease, it is possible that some disease cells (e.g., cancer cells) can be MTAP-deficient while others are not.

In a patient having or having been diagnosed with an MTA-accumulating disease, some cells may be MTA-accumulating while others are not.

An increase in therapeutic window between normal cells and MTAP-deleted/MTA accumulating cells could be achieved by using an inhibitor that binds PRMT5 uncompetitively with MTA. As used herein, “uncompetitive binding” and “uncompetitive inhibition” and “cooperative binding” and “cooperative inhibition” (e.g., MTA-uncompetitive binding, MTA-uncompetitive inhibition, MTA-cooperative binding, MTA-cooperative inhibition) refers to binding of an inhibitor to a protein (e.g., PRMT5) that is increased in the presence of a co-factor (e.g., MTA) over the binding of the same inhibitor in the absence of the co-factor. The PRMT5 inhibitors known in the art are generally either SAM (S-adenosylmethionine) uncompetitive or SAM competitive. As the concentration of SAM in wild-type and MTAP-null cells is similar, these inhibitors are expected to bind with similar potency to both cell types. By contrast, an MTA-cooperative (and either SAM competitive or showing enhanced cooperativity with MTA relative to SAM) inhibitor would bind with apparent greater potency in the presence of high concentrations of MTA and would therefore result in preferential inhibition of PRMT5 in MTA-accumulating cells relative to normal cells.

As described further herein, a cancer cell, a cancer type, or a subject with cancer, is “PRMT5 inhibitor sensitive,” sensitive to treatment with PRMT5 inhibitors,” sensitive to PRMT5 therapeutic inhibition,” or described in similar terms if it is amenable to treatment with a PRMT5 inhibitor, e.g., due to its MTAP deficiency and/or MTA accumulation character.

### PRMT5

“PRMT5” as used herein is the gene or protein Protein Arginine Methyltransferase 5, also known as HRMT1L5; IBP72; JBP1; SKB1; or SKB1Hs External IDs: OMIM: 604045, MGI: 1351645, HomoloGene: 4454, ChEMBL: 1795116, GeneCards: PRMT5 Gene; EC number 2.1.1.125. Ensembl ENSG00000100462; UniProt 014744; Entrez Gene ID: 10419; RefSeq (mRNA): NM_001039619. The mouse homolog is NM_013768. Methyltransferases such as PRMT5 catalyze the transfer of one to three methyl groups from the co-factor S-adenosylmethionine (also known as SAM or AdoMet) to lysine or arginine residues of histone proteins. Arginine methylation is carried out by 9 different protein arginine methyltransferases (PRMT) in humans. Three types of methylarginine species exist: (1) Monomethylarginine (MMA); (2) Asymmetric dimethyl arginine (ADMA), which is produced by Type I methyl transferases (PRMT1, PRMT2, PRMT3, CARM1, PRMT6 and PRMT8); and (3) Symmetrical dimethylarginine (SDMA), which is produced by Type II methyl transferases (PRMT5 and PRMT7). PRMT1 and PRMT5 are the major asymmetric and symmetric arginine methyltransferases, respectively. PRMT5 promotes symmetric dimethylation on histones at H3R8 and H4R3 (H4R3me2). Symmetric methylation of H4R3 is associated with transcriptional repression and can act as a binding site for DNMT3A. Loss of PRMT5 results in reduced DNMT3A binding and gene activation. Tumor suppressor gene ST7 and chemokines RNATES, IP10, CXCL11 are targeted and silenced by PRMT5. WO 2011/079236.

Additional substrates include E2F1, p53, EGFR and CRAF. PRMT5 is part of a multi-protein complex comprising the co-regulatory factor WDR77 (also known as MEP50, a CDK4 substrate) during G1/S transition. Phosphorylation increases PRMT5/WDR77 activity. WDR77 is the non-catalytic component of the complex and mediates interactions with binding partners and substrates. PRMT5 can also interact with pICIn or RioK1 adaptor proteins in a mutually exclusive fashion to modulate complex composition and substrate specificity.

PRMT5 has either a positive or negative effect on its substrates by arginine methylation when interacting with a number of complexes and is involved in a variety of cellular processes, including RNA processing, signal transduction, transcriptional regulation, and germ cell development. PRMT5 is a major pro-survival factor regulating eIF4E expression and p53 translation. PRMT5 triggers p53-dependent apoptosis and sensitized various cancer cells to Tumor necrosis factor (TNF)-related apoptosis-inducing ligand (TRAIL) without affecting TRAIL resistance in non-transformed cells.

The term “PRMT5 inhibitor” refers to any compound capable of inhibiting the production, level, activity, expression or presence of PRMT5. These include, as non-limiting examples, any compound inhibiting the transcription of the gene, the maturation of RNA, the translation of mRNA, the posttranslational modification of the protein, the enzymatic activity of the protein, the interaction of same with a substrate, etc. The term also refers to any agent that inhibits the cellular function of the PRMT5 protein, either by ATP-competitive inhibition of the active site, allosteric modulation of the protein structure, disruption of protein-protein interactions, or by inhibiting the transcription, translation, post-translational modification, or stability of PRMT5 protein.

In some embodiments, a PRMT5 inhibitor competes with another compound, protein or other molecule which interacts with PRMT5 and is necessary for PRMT5 function. As a non-limiting example, a PRMT5 inhibitor can compete with the co-factor S-adenosylmethionine (also known as SAM or AdoMet).

In some embodiments, the PRMT5 inhibitor is uncompetitive with MTA. In some embodiments, the PRMT5 inhibitor is uncompetitive with MTA and competitive with SAM. In some embodiments, the PRMT5 inhibitor is uncompetitive with MTA and uncompetitive with SAM but binds with a higher degree of potency for the MTA complex relative to the SAM complex.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). Additionally encompassed are compounds described herein as individual isomers substantially free of other isomers, and alternatively, as mixtures of various isomers.

The “enantiomeric excess” (“e.e.”) or “% enantiomeric excess” (“% e.e.”) of a composition as used herein refers to an excess of one enantiomer relative to the other enantiomer present in the composition. For example, a composition can contain 90% of one enantiomer, e.g., the S enantiomer, and 10% of the other enantiomer, i.e., the R enantiomer.

$$e . e . = ( 90 - 10 ) / 100 = 80 \% .$$

Thus, a composition containing 90% of one enantiomer and 10% of the other enantiomer is said to have an enantiomeric excess of 80%.

The “diastereomeric excess” (“d.e.”) or “% diastereomeric excess” (“% d.e.”) of a composition as used herein refers to an excess of one diastereomer relative to one or more different diastereomers present in the composition. For example, a composition can contain 90% of one diastereomer, and 10% of one or more different diastereomers.

$$d . e . = ( 90 - 10 ) / 100 = 80 \% .$$

Thus, a composition containing 90% of one diastereomers and 10% of one or more different diastereomers is said to have a diastereomeric excess of 80%.

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

In a formula, [symbol] is a single bond where the stereochemistry of the moieties immediately attached thereto is not specified.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

The term “azido” refers to the radical —N₃.

“Aliphatic” refers to an alkyl, alkenyl, alkynyl, or carbocyclyl group, as defined herein. “Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like. “Aralkyl” or “arylalkyl” is a subset of alkyl and aryl, as defined herein, and refers to an optionally substituted alkyl group substituted by an optionally substituted aryl group. “Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), ⁱPr (—CH(CH₃)₂), ⁿPr (—CH₂CH₂CH₃), ⁿBu (—CH₂CH₂CH₂CH₃), or ⁱBu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like. When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”).

Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl. Exemplary heteroalkyl groups include: —CH₂OH, —CH₂OCH₃, —CH₂NH₂, —CH₂NH(CH₃), —CH₂N(CH₃)₂, —CH₂CH₂OH, —CH₂CH₂OCH₃, —CH₂CH₂NH₂, —CH₂CH₂NH(CH₃), —CH₂CH₂N(CH₃)₂.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group is substituted with one or more of groups selected from halo, C₁-C₈ alkyl, C₁-C₈ haloalkyl, cyano, hydroxy, C₁-C₈ alkoxy, and amino.

Examples of representative substituted aryls include the following

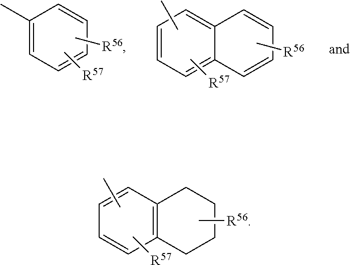

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group consisting of N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbons in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, In such instances, unless otherwise specified, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In some embodiments, a heteroaryl group is a bicyclic 8-12 membered aromatic ring system having ring carbon atoms and 1-6 ring heteroatoms provided in the aromatic ring system, wherein each heteroatom is independently selected from nitrogen, oxygen, and sulfur (“8-12 membered bicyclic heteroaryl”). In some embodiments, a heteroaryl group is an 8-10 membered bicyclic aromatic ring system having ring carbon atoms and 1-6 ring heteroatoms provided in the aromatic ring system, wherein each heteroatom is independently selected from nitrogen, oxygen, and sulfur (“8-10 membered bicyclic heteroaryl”). In some embodiments, a heteroaryl group is a 9-10 membered bicyclic aromatic ring system having ring carbon atoms and 1-6 ring heteroatoms provided in the aromatic ring system, wherein each heteroatom is independently selected from nitrogen, oxygen, and sulfur (“9-10 membered bicyclic heteroaryl”). Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

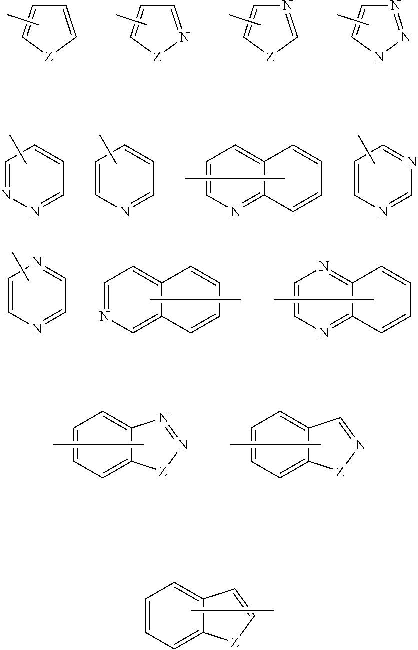

wherein each Z is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

In the structures described herein, a substituent attached to a polycyclic (e.g., bicyclic or tricyclic) cycloalkyl, heterocyclyl, aryl or heteroaryl with a bond that spans two or more rings is understood to mean that the substituent can be attached at any position in each of the rings.

“Heteroaralkyl” or “heteroarylalkyl” is a subset of “alkyl” and refers to an alkyl group substituted by a heteroaryl group, wherein the point of attachment is on the alkyl moiety. The term “carbocyclyl” or “carbocyclic” refers to a radical of a non-aromatic monocyclic, bicyclic, or tricyclic or polycyclic hydrocarbon ring system having from 3 to 14 ring carbon atoms (“C₃₋₁₄ carbocyclyl”) and zero heteroatoms in the non-aromatic ring system. Carbocyclyl groups include fully saturated ring systems (e.g., cycloalkyls), and partially saturated ring systems. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “cycloalkyl” as employed herein includes saturated cyclic, bicyclic, tricyclic, or polycyclic hydrocarbon groups having 3 to 14 carbons containing the indicated number of rings and carbon atoms (for example a C₃-C₁₄ monocyclic, C₄-C₁₄ bicyclic, C₅-C₁₄ tricyclic, or C₆-C₁₄ polycyclic cycloalkyl). In some embodiments “cycloalkyl” is a monocyclic cycloalkyl. In some embodiments, a monocyclic cycloalkyl has 3-14 ring carbon atoms. (“C₃₋₁₄ monocyclic cycloalkyl”). In some embodiments, a monocyclic cycloalkyl group has 3 to 10 ring carbon atoms (“C₃₋₁₀ monocyclic cycloalkyl”). In some embodiments, a monocyclic cycloalkyl group has 3 to 8 ring carbon atoms (“C₃₋₈ monocyclic cycloalkyl”). In some embodiments, a monocyclic cycloalkyl group has 3 to 6 ring carbon atoms (“C₃₋₆ monocyclic cycloalkyl”). In some embodiments, a monocyclic cycloalkyl group has 4 to 6 ring carbon atoms (“C₄₋₆ monocyclic cycloalkyl”). In some embodiments, a monocyclic cycloalkyl group has 5 to 6 ring carbon atoms (“C₅₋₆ monocyclic cycloalkyl”). In some embodiments, a monocyclic cycloalkyl group has 5 to 10 ring carbon atoms (“C₅₋₁₀ monocyclic cycloalkyl”). Examples of monocyclic C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈).

In some embodiments “cycloalkyl” is a bicyclic cycloalkyl. In some embodiments, a bicyclic cycloalkyl has 4-14 ring carbon atoms. (“C₄₋₁₄ bicyclic cycloalkyl”). In some embodiments, a bicyclic cycloalkyl group has 4 to 12 ring carbon atoms (“C₄₋₁₂ bicyclic cycloalkyl”). In some embodiments, a bicyclic cycloalkyl group has 4 to 10 ring carbon atoms (“C₄₋₁₀ bicyclic cycloalkyl”). In some embodiments, a bicyclic cycloalkyl group has 5 to 10 ring carbon atoms (“C₅₋₁₀ bicyclic cycloalkyl”). In some embodiments, a bicyclic cycloalkyl group has 6 to 10 ring carbon atoms (“C₆₋₁₀ bicyclic cycloalkyl”). In some embodiments, a bicyclic cycloalkyl group has 8 to 10 ring carbon atoms (“C₈₋₁₀ bicyclic cycloalkyl”). In some embodiments, a bicyclic cycloalkyl group has 7 to 9 ring carbon atoms (“C₇₋₉ bicyclic cycloalkyl”). Examples of bicyclic cycloalkyls include bicyclo[1.1.0]butane (C₄), bicyclo[1.1.1]pentane (C₅), spiro[2.2]pentane (C₅), bicyclo[2.1.0]pentane (C₅), bicyclo[2.1.1]hexane (C₆), bicyclo[3.1.0]hexane (C₆), spiro[2.3]hexane (C₆), bicyclo[2.2.1]heptane (norbornane) (C₇), bicyclo[3.2.0]heptane (C₇), bicyclo[3.1.1]heptane (C₇), bicyclo[3.1.1]heptane (C₇), bicyclo[4.1.0]heptane (C₇), spiro[2.4]heptane (C₇), spiro [3.3]heptane (C₇), bicyclo[2.2.2]octane (C₈), bicyclo[4.1.1]octane (C₈)octahydropentalene (C₈), bicyclo[3.2.1]octane (C₈), bicyclo[4.2.0]octane (C₈), spiro[2.5]octane (C₈), spiro[3.4]octane (C₈), bicyclo[3.3.1]nonane (C₉), octahydro-1H-indene (C₉), bicyclo[4.2.1]nonane (C₉), spiro[3.5]nonane (C₉), spiro[4.4]nonane (C₉), bicyclo[3.3.2]decane (C₁₀), bicyclo[4.3.1]decane (C₁₀), spiro[4.5]decane (C₁₀), bicyclo[3.3.3]undecane (C₁₁), decahydronaphthalene (C₁₀), bicyclo[4.3.2]undecane (C₁₁), spiro[5.5]undecane (C₁₁) and bicyclo[4.3.3]dodecane (C₁₂).

In some embodiments “cycloalkyl” is a tricyclic cycloalkyl. In some embodiments, a tricyclic cycloalkyl has 6-14 ring carbon atoms. (“C₆₋₁₄ tricyclic cycloalkyl”). In some embodiments, a tricyclic cycloalkyl group has 8 to 12 ring carbon atoms (“C₈₋₁₂ tricyclic cycloalkyl”). In some embodiments, a tricyclic cycloalkyl group has 10 to 12 ring carbon atoms (“C₁₀₋₁₂ tricyclic cycloalkyl. Examples of tricyclic cycloalkyls include adamantine (C₁₂).

Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like.

Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. “Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(═O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(═O)—C₁-C₈ alkyl, —C(═O)—(CH₂)ₜ(C₆-C₁₀ aryl), —C(═O)—(CH₂)ₜ(5-10 membered heteroaryl), —C(═O)—(CH₂)ₜ(C₃-C₁₀ cycloalkyl), and —C(═O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

The term aminoalkyl refers to a substituted alkyl group wherein one or more of the hydrogen atoms are independently replaced by an —NH₂ group.

The term hydroxyalkyl refers to a substituted alkyl group wherein one or more of the hydrogen atoms are independently replaced by an —OH group.

The terms “alkylamino” and “dialkylamino” refer to —NH(alkyl) and —N(alkyl)₂ radicals respectively. In some embodiments the alkylamino is a —NH(C₁-C₄ alkyl). In some embodiments the alkylamino is methylamino, ethylamino, propylamino, isopropylamino, n-butylamino, iso-butylamino, sec-butylamino or tert-butylamino. In some embodiments the dialkylamino is —N(C₁-C₆ alkyl)₂. In some embodiments the dialkylamino is a dimethylamino, a methylethylamino, a diethylamino, a methylpropylamino, a methylisopropylamino, a methylbutylamino, a methylisobutylamino or a methyltertbutylamino.

The term “aryloxy” refers to an —O-aryl radical. In some embodiments the aryloxy group is phenoxy.

The term “haloalkoxy” refers to alkoxy structures that are substituted with one or more halo groups or with combinations thereof. For example, the term “fluoroalkoxy” includes haloalkoxy groups, in which the halo is fluorine. In some embodiments haloalkoxy groups are difluoromethoxy and trifluoromethoxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂N(CH₃)₂.

“Amino” refers to the radical —NH₂.

“Oxo group” refers to —C(═O)—.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —C₁₋₁₀ alkyl (e.g., aralkyl, heteroaralkyl), —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.
each instance of R^{aa} is, independently, selected from —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{d}d groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion.
each instance of R^{cc} is, independently, selected from hydrogen, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NRC(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂—P(═O)(R^{ee})₂, —OP(═O)(R^{ee})₂, —OP(═O)(OR^{ee})₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from —C₁₋₆ alkyl, —C₁₋₆ perhaloalkyl, —C₂₋₆ alkenyl, —C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “leaving group” is given its ordinary meaning in the art of synthetic organic chemistry and refers to an atom or a group capable of being displaced by a nucleophile. Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In certain embodiments, the leaving group is halogen, alkanesulfonyloxy, arenesulfonyloxy, diazonium, alkyl diazenes, aryl diazenes, alkyl triazenes, aryl triazenes, nitro, alkyl nitrate, aryl nitrate, alkyl phosphate, aryl phosphate, alkyl carbonyl oxy, aryl carbonyl oxy, alkoxcarbonyl oxy, aryoxcarbonyl oxy ammonia, alkyl amines, aryl amines, hydroxyl group, alkyloxy group, or aryloxy. In some cases, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

“Carboxy” refers to the radical —C(═O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl (—CF₃), difluoromethyl (—CHF₂), fluoromethyl (—CH₂F), chloromethyl (—CH₂Cl), dichloromethyl (—CHCl₂), tribromomethyl (—CH₂Br), and the like.

“Hydroxy” refers to the radical —OH.

“Nitro” refers to the radical —NO₂.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. Any and all such combinations are contemplated in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc}), —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —S(═O)(═NR^{bb})R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc}), —P(R^{cc})₃, —OP(R^{cc})₂—OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R′)₂, —CN, —C(═O)R^{aa}, —C(═O)N(R^{cc})₂, —CO₂R^{aa}, —SO₂R^{aa}, —C(═NR^{cc})OR^{aa}, —C(═NRC)N(RC)₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R)₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(Re)₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of W is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of Rn is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R′)₂, —CN, —C(═O)R^{aa}, —C(═O)N(R)₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

As used herein, the term “salt” refers to any and all salts and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomologus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In one embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response e.g., to treat a disease or disorder described herein. As will be appreciated by those of ordinary skill in this art, the effective amount of a compound of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the therapeutic treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the therapeutic treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Compounds

In one embodiment, provided is a compound selected from the compounds of Table 1, or pharmaceutically acceptable salts thereof.

In one aspect of the invention, provided is a compound of Formula (I) or a pharmaceutically acceptable salt thereof,

wherein:

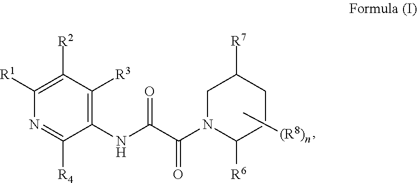

each R¹ is independently selected from H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
each R² is independently selected from halo, —CN, —C₁-C₆ alkyl, —C₁-C₆ heteroalkyl, —C₁-C₆ haloalkyl, —C₁-C₆ haloalkoxy, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁶ is independently absent or selected from H, -D, halo, —CN, —C₁-C₆ alkyl, —C₁-C₆ heteroalkyl, —C₁-C₆ haloalkyl, —C₃-C₁₀ carbocyclyl, 3-10 membered heterocyclyl, heterocyclylalkyl, C₆-C₁₀ aryl, 5-10 member heteroaryl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR⁶, —S(═O)(═NR^{a6})R^{a6}, —NR^{a6}S(═O)₂R^{a6} and —S(═O)₂N(R^{a6})₂, wherein each alkyl, carbocyclyl, heterocyclyl, cycloalkylalkyl, heterocyclylalkyl, aryl, heteroaryl, arylalkyl and heteroarylalkyl is optionally substituted at any available position;
each R⁷ is independently absent or selected from H, -D, halo, —CN, —C₁-C₆ alkyl, —C₁-C₆ hydroxyalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a7}, —N(R^{a7})₂, —C(═O)R^{a7}, —C(═O)OR^{a7}, —NR^{a7}C(═O)R^{a7}, —NR^{a7}C(═O)OR^{a7}, —C(═O)N(R^{a7})₂, —OC(═O)N(R^{a7})₂, —S(═O)R^{a7}, —S(═O)₂R^{a7}, —SR^{a7}, —S(═O)(═NR^{a7})R^{a7}, —NR^{a7}S(═O)₂R^{a7} and —S(═O)₂N(R^{a7})₂;
each R⁸ is independently selected from H, -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a8}, —N(R^{a8})₂, —C(═O)R^{a8}, —C(═O)OR^{a8}, —NR^{a8}C(═O)R^{a8}, —NR^{a8}C(═O)OR^{a8}, —CH₂C(═O)N(R^{a8})₂—C(═O)N(R^{a8})₂, —OC(═O)N(R^{a8})₂, —CH₂C(═O)N(R^{a8})₂, —S(═O)R^{a8}, —S(═O)₂R^{a8}, —SR^{a8}, —S(═O)(═NR^{a8})R^{a8}, —NR^{a8}S(═O)₂R^{as} and —S(═O)₂N(R^{a8})₂ wherein two instances of R⁸ together with the atom or atoms to which they are attached can be taken together to form a 3-10 member cycloalkyl or heterocyclyl ring (e.g., a ring that together with the piperidine ring of Structure I can form a bridged, fused or spiro bicyclic heterocyclic ring)
each R^{a1}, R^{a2}, R^{a3}, R^{a4}, R^{a6}, R^{a7} and R^{as} is independently selected from H, C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R9, wherein each R9 is independently selected from ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from H, —C₁-C₆ alkyl (e.g., -Me, -Et, —Pr, —ⁱPr, -ⁿBu, -^{t}Bu, -sec-Bu, -iso-Bu). and C₃-C₉ cycloalkyl (e.g., cyclopropyl, cyclobutyl, cyclopentyl, cyclohexyl); and
n is 0, 1, 2 or 3 and wherein the compound is selected from the compounds of Table 1.

Compounds described herein (e.g., a compound of Table 1, or pharmaceutically acceptable salts thereof) are useful as inhibitors of PRMT5 (e.g., MTA uncompetitive PRMT5 inhibitors).

Table 1 indicates IC₅₀ and IC₉₀ values in an MTAP-isogenic cell line pair for exemplary compounds in an SDMA in-cell western assay (described in Example 97) (columns 4-6). HAP1 MTAP-intact is a cell line in which endogenous levels of MTAP are expressed, and HAP1 MTAP-deleted is an MTAP-null cell line. For Table 1, “a” and “aa” indicates an IC₅₀ of <5 nM, “b” and “bb” indicates an IC₅₀ equal to or greater than 5 nM but less than 50 nM, and “c” and “cc” indicates an IC₅₀ of greater than or equal to 50 nM in the HAP1 MTAP-intact (column 4) and the HAP1 MTAP-deleted (column 5) assays, respectively. Similarly, “aaa” indicates an IC₉₀ of <75 nM, “bbb” indicates an IC₉₀ equal to or greater than 75 nM but less than 125 nM, and “ccc” indicates an IC₉₀ of greater than or equal to 125 nM in the HAP1 MTAP-deleted (column 6) assay.

In column 7, “A” indicates an IC₅₀ ratio greater than or equal to 30 fold between the IC₅₀ in the HAP1 MTAP-intact cell line and the HAP1 MTAP-deleted cell line; “B” indicates an IC₅₀ ratio greater than or equal to 15 fold but lower than 30 fold between the IC₅₀ in the HAP1 MTAP-intact cell line and the HAP1 MTAP-deleted cell line; “C” indicates an IC₅₀ ratio of less than 15 fold between the IC₅₀ in the HAP1 MTAP-intact cell line and the HAP1 MTAP-deleted cell line. Compounds with a ratio in the SDMA in-cell western assay of equal to or greater than 3 fold are considered MTAP-selective.

Table 1 additionally indicates IC₅₀ values in a viability assay for the MTAP-deleted cell line (described in Example 98) (column 8), indicating the effect of treatment with compound on cell survival. In column 8, a value of A* indicates an IC₅₀ of less than 100 nM, a value of B* indicates an IC₅₀ equal to or greater than 100 nM but less than 1 μM, and a value of C* indicates an IC₅₀ greater than or equal to 1 μM.

Unless otherwise indicated, the absolute stereochemistry of all chiral atoms is as depicted. Compounds marked with (or) or (rel) are single enantiomers wherein the absolute stereochemistry was arbitrarily assigned (e.g., based on chiral SFC elution as described in the Examples section). Compounds marked with (and) or (rac) are mixtures of enantiomers wherein the relative stereochemistry is as shown. Compounds that have a stereogenic center where the configuration is not indicated in the structure as depicted and that are not marked in the “stereochemistry” column are mixtures of enantiomers. Compounds marked with (abs) are single enantiomers wherein the absolute stereochemistry is as indicated. In some instances, different indicators selected from (abs) (or) and (and) apply to different portions of the molecule. A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

TABLE 1
Exemplary compounds and biological data

| | | | | | | Hap1 | Hap1 |
|---|---|---|---|---|---|---|---|
| | | | Hap1 | Hap1 | Hap1 | MTAP | MTAP |
| | | | MTAP | MTAP | MTAP | intact/ | deleted |
| | Stereo | | intact | deleted | deleted | deleted | viability |
| STRUCTURE | chemistry | Nr | IC50 | IC50 | IC90 | ratio | IC50 |
| 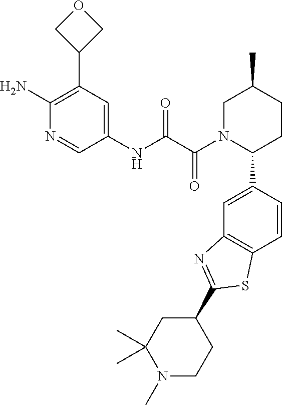 [SMILES: C[C@H]1CC[C@H](c2ccc3sc([C@H]4CCN(C)C(C)(C)C4)nc3c2)N(C(=O)C(=O)Nc2cnc(N)c(C3COC3)c2)C1] | (abs) on core, (or) sidechain | 1 | b | aa | aaa | C | A* |
| 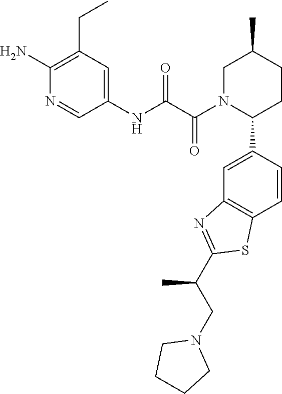 [SMILES: CCc1cc(NC(=O)C(=O)N2C[C@@H](C)CC[C@@H]2c2ccc3sc([C@H](C)CN4CCCC4)nc3c2)cnc1N] | (abs) core, (or) sidechain | 2 | b | aa | aaa | B | A* |
| 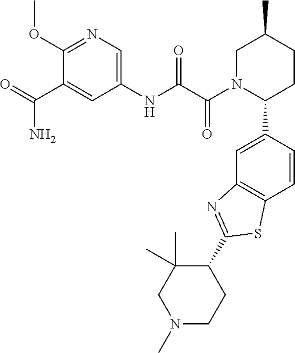 [SMILES: COc1ncc(NC(=O)C(=O)N2C[C@@H](C)CC[C@@H]2c2ccc3sc([C@@H]4CCN(C)CC4(C)C)nc3c2)cc1C(N)=O] | (abs) core, (or) sidechain | 3 | c | aa | aaa | B | A* |
| 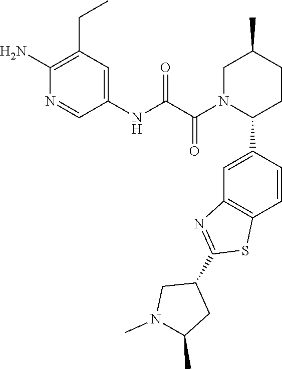 [SMILES: CCc1cc(NC(=O)C(=O)N2C[C@@H](C)CC[C@@H]2c2ccc3sc([C@@H]4C[C@@H](C)N(C)C4)nc3c2)cnc1N] | (abs) core (and) sidechain | 4 | c | aa | aaa | A | A* |
| 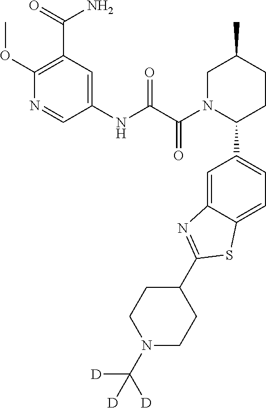 [SMILES: [2H]C([2H])([2H])N1CCC(c2nc3cc([C@H]4CC[C@H](C)CN4C(=O)C(=O)Nc4cnc(OC)c(C(N)=O)c4)ccc3s2)CC1] | (abs) | 5 | b | aa | aaa | A | A* |
| 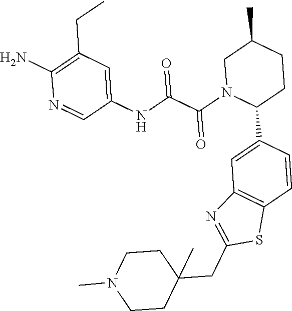 [SMILES: CCc1cc(NC(=O)C(=O)N2C[C@@H](C)CC[C@@H]2c2ccc3sc(CC4(C)CCN(C)CC4)nc3c2)cnc1N] | (abs) | 6 | b | aa | aaa | B | A* |
| 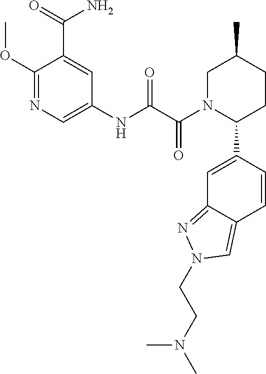 [SMILES: COc1ncc(NC(=O)C(=O)N2C[C@@H](C)CC[C@@H]2c2ccc3cn(CCN(C)C)nc3c2)cc1C(N)=O] | (abs) | 7 | c | bb | aaa | A | A* |
| 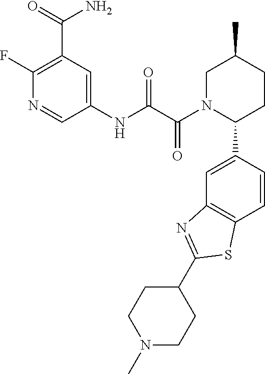 [SMILES: C[C@H]1CC[C@H](c2ccc3sc(C4CCN(C)CC4)nc3c2)N(C(=O)C(=O)Nc2cnc(F)c(C(N)=O)c2)C1] | (abs) | 8 | c | aa | aaa | B | A* |
| 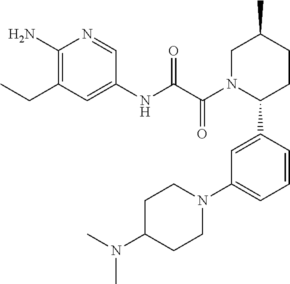 [SMILES: CCc1cc(NC(=O)C(=O)N2C[C@@H](C)CC[C@@H]2c2cccc(N3CCC(N(C)C)CC3)c2)cnc1N] | (abs) | 9 | c | aa | aaa | A | A* |
| 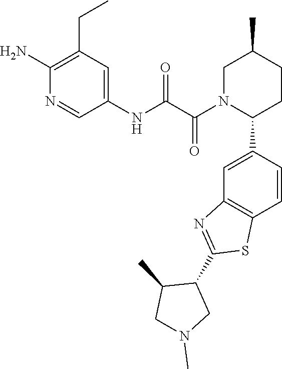 [SMILES: CCc1cc(NC(=O)C(=O)N2C[C@@H](C)CC[C@@H]2c2ccc3sc([C@@H]4CN(C)C[C@H]4C)nc3c2)cnc1N] | (abs) core, (and) sidechain | 10 | c | aa | aaa | A | A* |
| 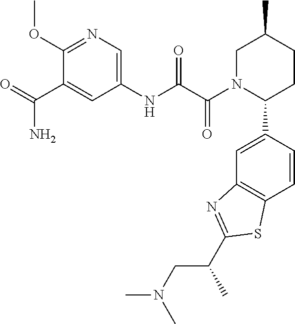 [SMILES: COc1ncc(NC(=O)C(=O)N2C[C@@H](C)CC[C@@H]2c2ccc3sc([C@H](C)CN(C)C)nc3c2)cc1C(N)=O] | (abs) core, (or) sidechain | 11 | c | aa | aaa | A | A* |
| 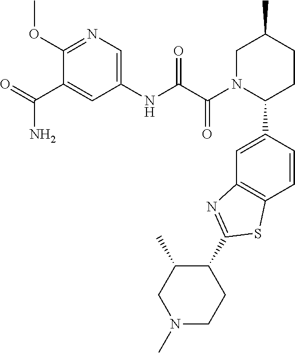 [SMILES: COc1ncc(NC(=O)C(=O)N2C[C@@H](C)CC[C@@H]2c2ccc3sc([C@@H]4CCN(C)C[C@@H]4C)nc3c2)cc1C(N)=O] | (abs) | 12 | c | aa | aaa | A | A* |
| 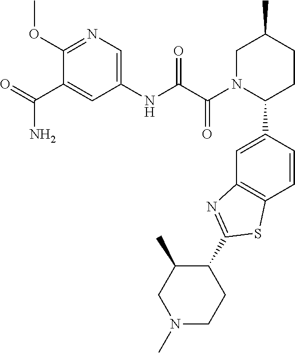 [SMILES: COc1ncc(NC(=O)C(=O)N2C[C@@H](C)CC[C@@H]2c2ccc3sc([C@@H]4CCN(C)C[C@H]4C)nc3c2)cc1C(N)=O] | (abs) core, (or) sidechain | Ent-12 | | | | | |
| 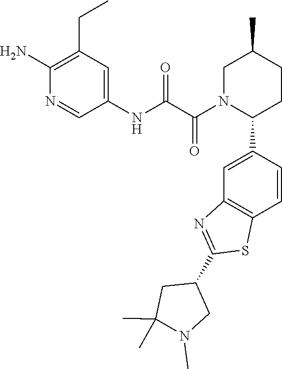 [SMILES: CCc1cc(NC(=O)C(=O)N2C[C@@H](C)CC[C@@H]2c2ccc3sc([C@@H]4CN(C)C(C)(C)C4)nc3c2)cnc1N] | (abs) core, (and) sidechain | 13 | b | aa | aaa | C | A* |
| 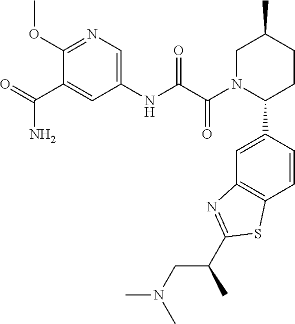 [SMILES: COc1ncc(NC(=O)C(=O)N2C[C@@H](C)CC[C@@H]2c2ccc3sc([C@@H](C)CN(C)C)nc3c2)cc1C(N)=O] | (abs) core, (or) sidechain | 14 | b | aa | aaa | B | A* |
| 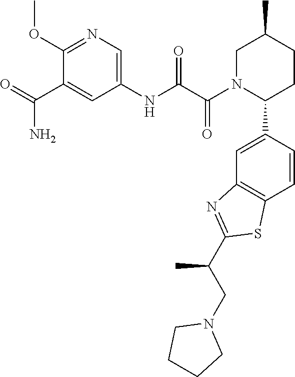 [SMILES: COc1ncc(NC(=O)C(=O)N2C[C@@H](C)CC[C@@H]2c2ccc3sc([C@H](C)CN4CCCC4)nc3c2)cc1C(N)=O] | (abs) core, (or) sidechain | 15 | C | aa | aaa | B | A* |
| 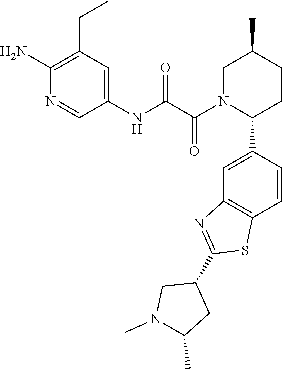 [SMILES: CCc1cc(NC(=O)C(=O)N2C[C@@H](C)CC[C@@H]2c2ccc3sc([C@@H]4C[C@H](C)N(C)C4)nc3c2)cnc1N] | (abs) core, (and) sidechain | 16 | c | aa | aaa | A | A* |
| 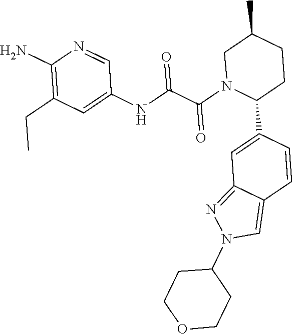 [SMILES: CCc1cc(NC(=O)C(=O)N2C[C@@H](C)CC[C@@H]2c2ccc3cn(C4CCOCC4)nc3c2)cnc1N] | (abs) | 17 | c | bb | aaa | A | A* |
| 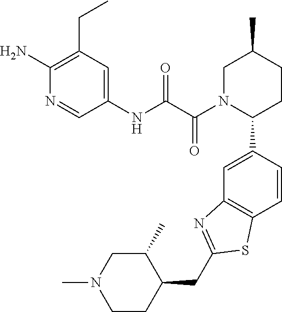 [SMILES: CCc1cc(NC(=O)C(=O)N2C[C@@H](C)CC[C@@H]2c2ccc3sc(C[C@H]4CCN(C)C[C@@H]4C)nc3c2)cnc1N] | (abs) core, (and) sidechain | 18 | b | aa | aaa | A | A* |
| 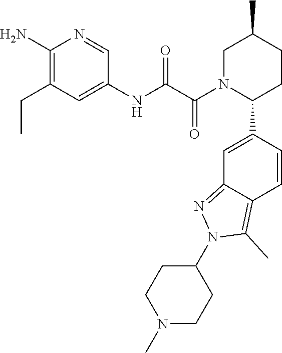 [SMILES: CCc1cc(NC(=O)C(=O)N2C[C@@H](C)CC[C@@H]2c2ccc3c(C)n(C4CCN(C)CC4)nc3c2)cnc1N] | (abs) | 19 | c | aa | aaa | A | A* |
| 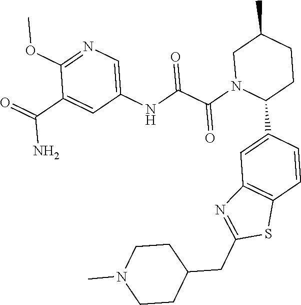 [SMILES: COc1ncc(NC(=O)C(=O)N2C[C@@H](C)CC[C@@H]2c2ccc3sc(CC4CCN(C)CC4)nc3c2)cc1C(N)=O] | (abs) | 20 | C | aa | aaa | A | A* |
| 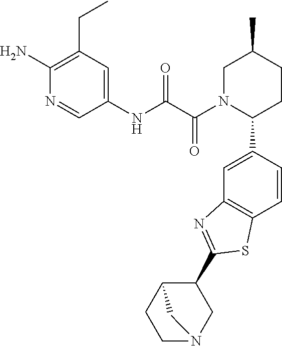 [SMILES: CCc1cc(NC(=O)C(=O)N2C[C@@H](C)CC[C@@H]2c2ccc3sc([C@H]4CN5CC[C@@H]4C5)nc3c2)cnc1N] | (abs) core, (and) sidechain | 21 | b | aa | aaa | C | A* |
| 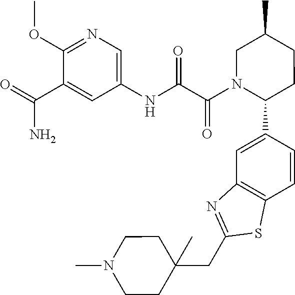 [SMILES: COc1ncc(NC(=O)C(=O)N2C[C@@H](C)CC[C@@H]2c2ccc3sc(CC4(C)CCN(C)CC4)nc3c2)cc1C(N)=O] | (abs) | 22 | c | aa | aaa | B | A* |
| 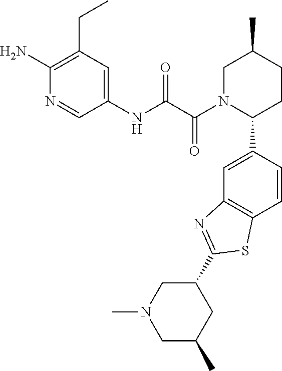 [SMILES: CCc1cc(NC(=O)C(=O)N2C[C@@H](C)CC[C@@H]2c2ccc3sc([C@@H]4C[C@@H](C)CN(C)C4)nc3c2)cnc1N] | (abs) core, (or) sidechain | 23 | b | aa | aaa | B | A* |
| 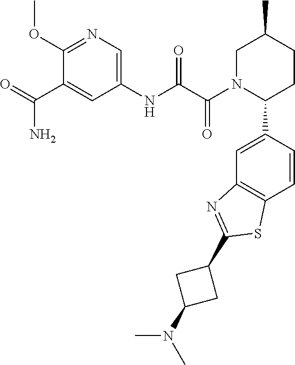 [SMILES: COc1ncc(NC(=O)C(=O)N2C[C@@H](C)CC[C@@H]2c2ccc3sc([C@H]4C[C@@H](N(C)C)C4)nc3c2)cc1C(N)=O] | (abs) core, (or) sidechain | 24 | c | aa | aaa | A | A* |
| 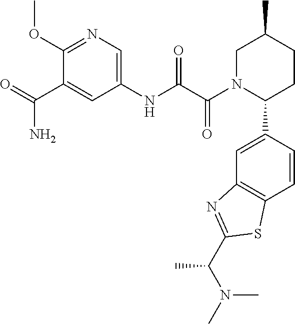 [SMILES: COc1ncc(NC(=O)C(=O)N2C[C@@H](C)CC[C@@H]2c2ccc3sc([C@@H](C)N(C)C)nc3c2)cc1C(N)=O] | (abs) core, (or) sidechain | 25 | c | bb | bbb | A | A* |
| 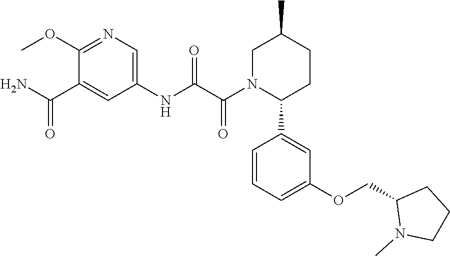 [SMILES: COc1ncc(NC(=O)C(=O)N2C[C@@H](C)CC[C@@H]2c2cccc(OC[C@@H]3CCCN3C)c2)cc1C(N)=O] | (abs) | 26 | c | bb | ccc | A | B* |
| 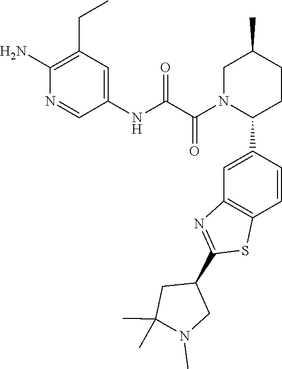 [SMILES: CCc1cc(NC(=O)C(=O)N2C[C@@H](C)CC[C@@H]2c2ccc3sc([C@H]4CN(C)C(C)(C)C4)nc3c2)cnc1N] | (abs) core, (or) sidechain | 27 | b | aa | aaa | B | A* |
| 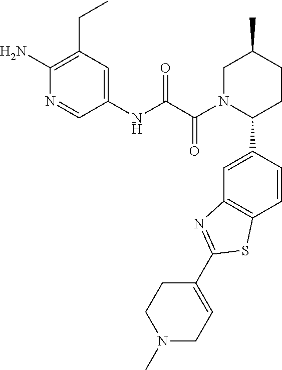 [SMILES: CCc1cc(NC(=O)C(=O)N2C[C@@H](C)CC[C@@H]2c2ccc3sc(C4=CCN(C)CC4)nc3c2)cnc1N] | (abs) | 28 | b | aa | aaa | A | A* |
| 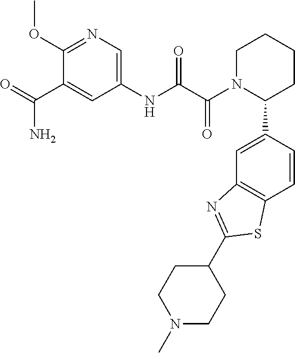 [SMILES: COc1ncc(NC(=O)C(=O)N2CCCC[C@@H]2c2ccc3sc(C4CCN(C)CC4)nc3c2)cc1C(N)=O] | (abs) | 29 | c | bb | ccc | A | B* |
| 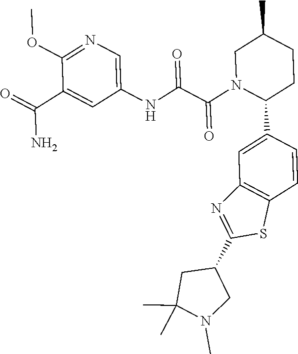 [SMILES: COc1ncc(NC(=O)C(=O)N2C[C@@H](C)CC[C@@H]2c2ccc3sc([C@@H]4CN(C)C(C)(C)C4)nc3c2)cc1C(N)=O] | (abs) core, (or) sidechain | 30 | c | aa | aaa | B | A* |
| 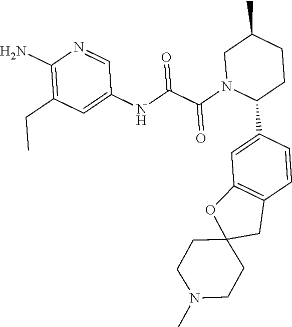 [SMILES: CCc1cc(NC(=O)C(=O)N2C[C@@H](C)CC[C@@H]2c2ccc3c(c2)OC2(CCN(C)CC2)C3)cnc1N] | (abs) | 31 | c | bb | ccc | A | B* |
| 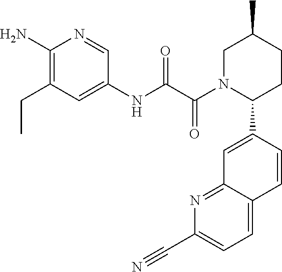 [SMILES: CCc1cc(NC(=O)C(=O)N2C[C@@H](C)CC[C@@H]2c2ccc3ccc(C#N)nc3c2)cnc1N] | (abs) | 32 | c | bb | ccc | A | B* |
| 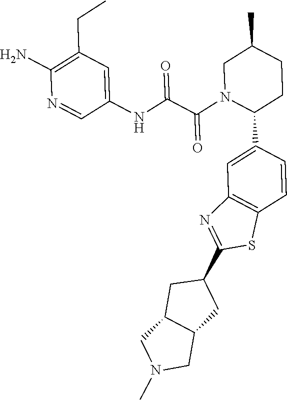 [SMILES: CCc1cc(NC(=O)C(=O)N2C[C@@H](C)CC[C@@H]2c2ccc3sc([C@H]4C[C@@H]5CN(C)C[C@@H]5C4)nc3c2)cnc1N] | (abs) core, (or) sidechain | 33 | c | aa | aaa | A | A* |
| 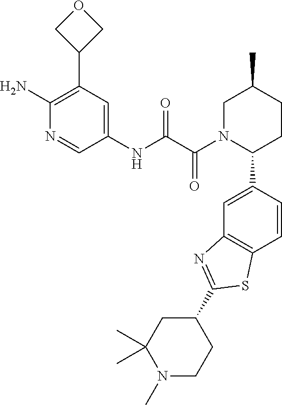 [SMILES: C[C@H]1CC[C@H](c2ccc3sc([C@@H]4CCN(C)C(C)(C)C4)nc3c2)N(C(=O)C(=O)Nc2cnc(N)c(C3COC3)c2)C1] | (abs) core, (or) sidechain | 34 | b | aa | aaa | C | A* |
| 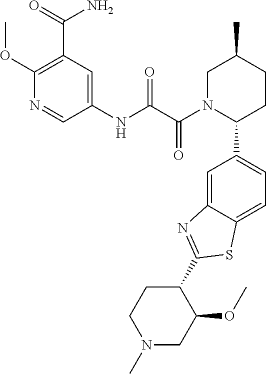 [SMILES: COc1ncc(NC(=O)C(=O)N2C[C@@H](C)CC[C@@H]2c2ccc3sc([C@H]4CCN(C)C[C@@H]4OC)nc3c2)cc1C(N)=O] | (abs) core, (or) sidechain | 35 | c | aa | aaa | A | A* |
| 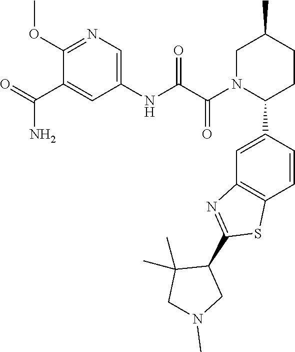 [SMILES: COc1ncc(NC(=O)C(=O)N2C[C@@H](C)CC[C@@H]2c2ccc3sc([C@H]4CN(C)CC4(C)C)nc3c2)cc1C(N)=O] | (abs) core, (or) sidechain | 36 | c | aa | aaa | A | A* |
| 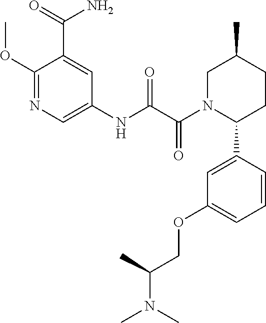 [SMILES: COc1ncc(NC(=O)C(=O)N2C[C@@H](C)CC[C@@H]2c2cccc(OC[C@H](C)N(C)C)c2)cc1C(N)=O] | (abs) | 37 | c | bb | bbb | A | A* |
| 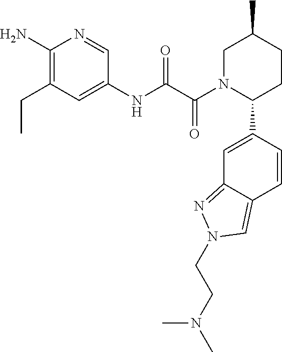 [SMILES: CCc1cc(NC(=O)C(=O)N2C[C@@H](C)CC[C@@H]2c2ccc3cn(CCN(C)C)nc3c2)cnc1N] | (abs) | 38 | c | aa | aaa | B | A* |
| 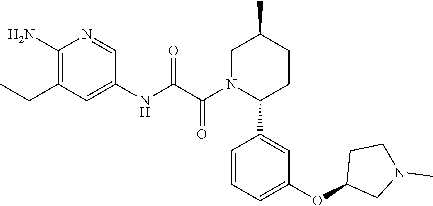 [SMILES: CCc1cc(NC(=O)C(=O)N2C[C@@H](C)CC[C@@H]2c2cccc(O[C@H]3CCN(C)C3)c2)cnc1N] | (abs) core, (and) sidechain | 39 | c | bb | bbb | A | A* |
| 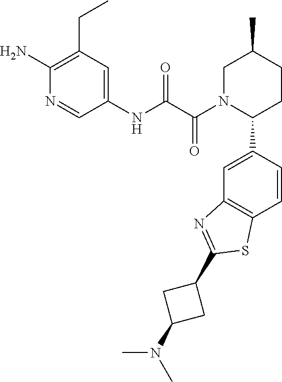 [SMILES: CCc1cc(NC(=O)C(=O)N2C[C@@H](C)CC[C@@H]2c2ccc3sc([C@H]4C[C@@H](N(C)C)C4)nc3c2)cnc1N] | (abs) core, (or) sidechain | 40 | c | aa | aaa | A | A* |
| 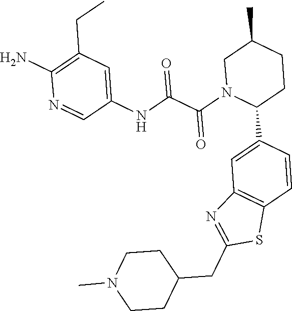 [SMILES: CCc1cc(NC(=O)C(=O)N2C[C@@H](C)CC[C@@H]2c2ccc3sc(CC4CCN(C)CC4)nc3c2)cnc1N] | (abs) | 41 | b | aa | aaa | B | A* |
| 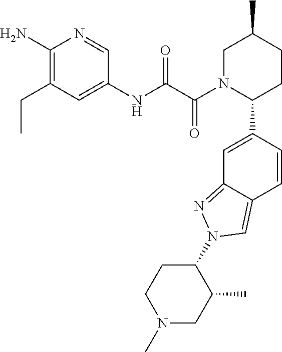 [SMILES: CCc1cc(NC(=O)C(=O)N2C[C@@H](C)CC[C@@H]2c2ccc3cn([C@H]4CCN(C)C[C@H]4C)nc3c2)cnc1N] | (abs) core, (or) sidechain | 42 | c | aa | aaa | B | A* |
| 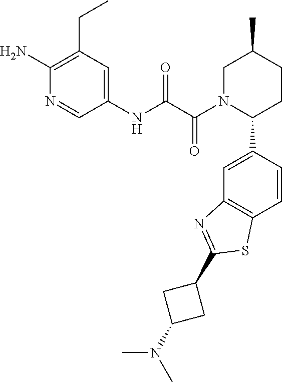 [SMILES: CCc1cc(NC(=O)C(=O)N2C[C@@H](C)CC[C@@H]2c2ccc3sc([C@H]4C[C@H](N(C)C)C4)nc3c2)cnc1N] | (abs) core, (or) sidechain | 43 | c | aa | aaa | A | A* |
| 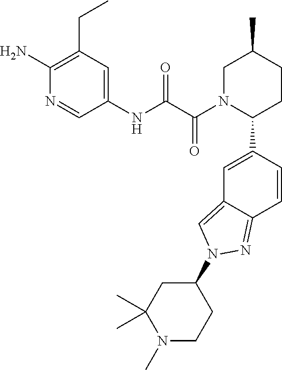 [SMILES: CCc1cc(NC(=O)C(=O)N2C[C@@H](C)CC[C@@H]2c2ccc3nn([C@H]4CCN(C)C(C)(C)C4)cc3c2)cnc1N] | (abs) core, (or) sidechain | 44 | b | aa | aaa | B | A* |
| 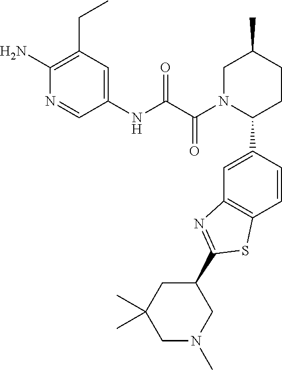 [SMILES: CCc1cc(NC(=O)C(=O)N2C[C@@H](C)CC[C@@H]2c2ccc3sc([C@H]4CN(C)CC(C)(C)C4)nc3c2)cnc1N] | (abs) core, (or) sidechain | 45 | b | aa | aaa | B | A* |
| 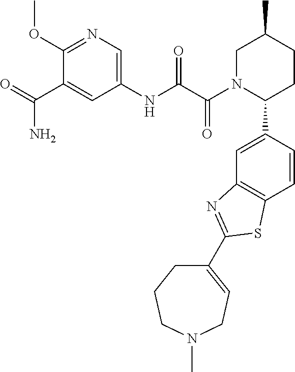 [SMILES: COc1ncc(NC(=O)C(=O)N2C[C@@H](C)CC[C@@H]2c2ccc3sc(C4=CCN(C)CCC4)nc3c2)cc1C(N)=O] | (abs) | 46 | b | aa | aaa | B | A* |
| 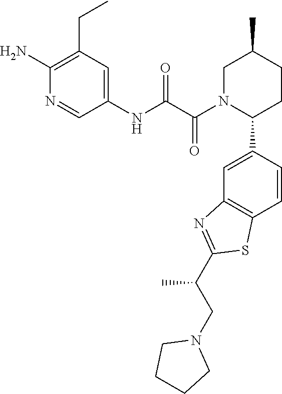 [SMILES: CCc1cc(NC(=O)C(=O)N2C[C@@H](C)CC[C@@H]2c2ccc3sc([C@@H](C)CN4CCCC4)nc3c2)cnc1N] | (abs) core, (and) sidechain | 47 | c | aa | aaa | A | A* |
| 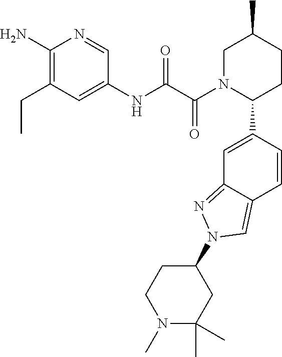 [SMILES: CCc1cc(NC(=O)C(=O)N2C[C@@H](C)CC[C@@H]2c2ccc3cn([C@@H]4CCN(C)C(C)(C)C4)nc3c2)cnc1N] | (abs) core, (or) sidechain | 48 | b | aa | aaa | C | A* |
| 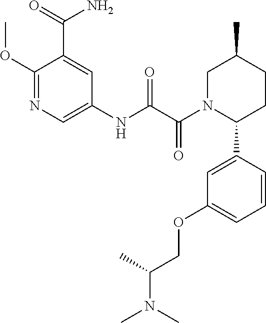 [SMILES: COc1ncc(NC(=O)C(=O)N2C[C@@H](C)CC[C@@H]2c2cccc(OC[C@@H](C)N(C)C)c2)cc1C(N)=O] | (abs) | 49 | c | aa | aaa | A | B* |
| 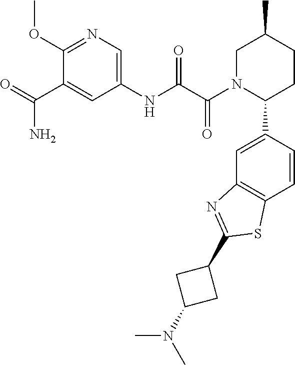 [SMILES: COc1ncc(NC(=O)C(=O)N2C[C@@H](C)CC[C@@H]2c2ccc3sc([C@H]4C[C@H](N(C)C)C4)nc3c2)cc1C(N)=O] | (abs) core, (or) sidechain | 50 | c | aa | aaa | A | A* |
| 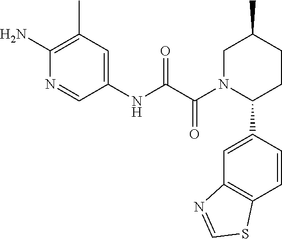 [SMILES: Cc1cc(NC(=O)C(=O)N2C[C@@H](C)CC[C@@H]2c2ccc3scnc3c2)cnc1N] | (abs) | 51 | c | aa | bbb | A | B* |
| 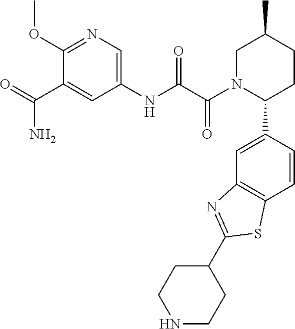 [SMILES: COc1ncc(NC(=O)C(=O)N2C[C@@H](C)CC[C@@H]2c2ccc3sc(C4CCNCC4)nc3c2)cc1C(N)=O] | (abs) | 52 | c | bb | bbb | B | B* |
| 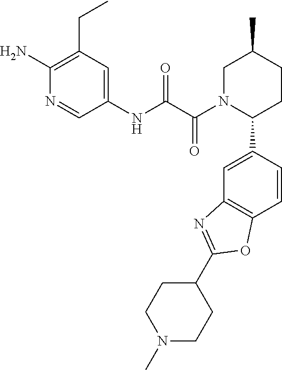 [SMILES: CCc1cc(NC(=O)C(=O)N2C[C@@H](C)CC[C@@H]2c2ccc3oc(C4CCN(C)CC4)nc3c2)cnc1N] | (abs) | 53 | c | aa | aaa | A | A* |
| 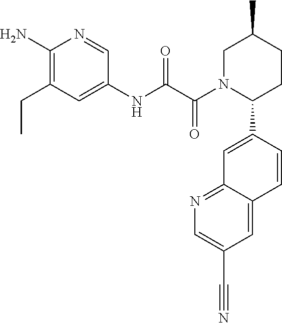 [SMILES: CCc1cc(NC(=O)C(=O)N2C[C@@H](C)CC[C@@H]2c2ccc3cc(C#N)cnc3c2)cnc1N] | (abs) | 54 | c | bb | ccc | A | B* |
| 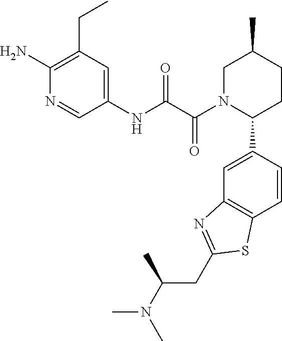 [SMILES: CCc1cc(NC(=O)C(=O)N2C[C@@H](C)CC[C@@H]2c2ccc3sc(C[C@H](C)N(C)C)nc3c2)cnc1N] | (abs) | 55 | b | aa | aaa | B | A* |
| 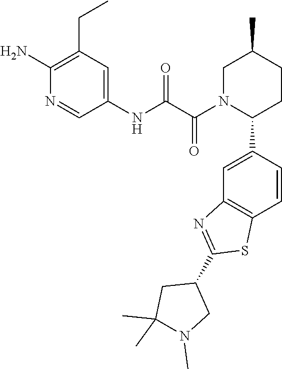 [SMILES: CCc1cc(NC(=O)C(=O)N2C[C@@H](C)CC[C@@H]2c2ccc3sc([C@@H]4CN(C)C(C)(C)C4)nc3c2)cnc1N] | (abs) core, (or) sidechain | 56 | b | aa | aaa | B | A* |
| 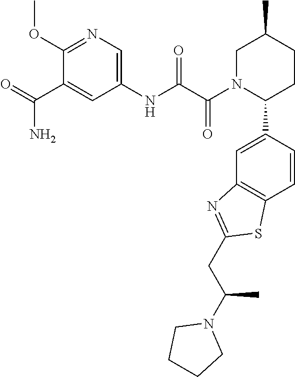 [SMILES: COc1ncc(NC(=O)C(=O)N2C[C@@H](C)CC[C@@H]2c2ccc3sc(C[C@@H](C)N4CCCC4)nc3c2)cc1C(N)=O] | (abs) core, (and) sidechain | 57 | c | aa | aaa | A | A* |
| 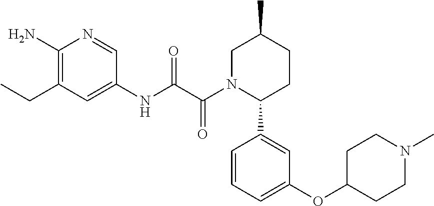 [SMILES: CCc1cc(NC(=O)C(=O)N2C[C@@H](C)CC[C@@H]2c2cccc(OC3CCN(C)CC3)c2)cnc1N] | (abs) | 58 | c | aa | aaa | A | A* |
| 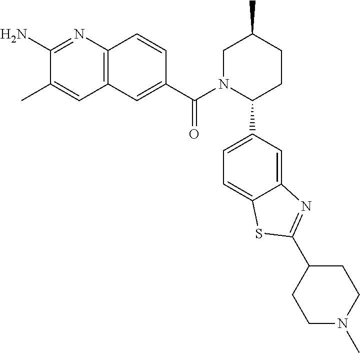 [SMILES: Cc1cc2cc(C(=O)N3C[C@@H](C)CC[C@@H]3c3ccc4sc(C5CCN(C)CC5)nc4c3)ccc2nc1N] | (abs) | 59 | c | aa | aaa | A | A* |
| 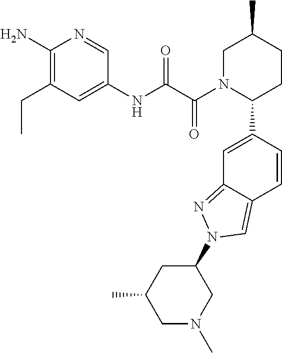 [SMILES: CCc1cc(NC(=O)C(=O)N2C[C@@H](C)CC[C@@H]2c2ccc3cn([C@@H]4C[C@@H](C)CN(C)C4)nc3c2)cnc1N] | (abs) core, (or) sidechain | 60 | c | aa | aaa | A | A* |
| 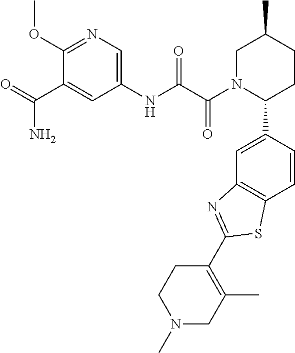 [SMILES: COc1ncc(NC(=O)C(=O)N2C[C@@H](C)CC[C@@H]2c2ccc3sc(C4=C(C)CN(C)CC4)nc3c2)cc1C(N)=O] | (abs) | 61 | c | aa | aaa | A | A* |
| 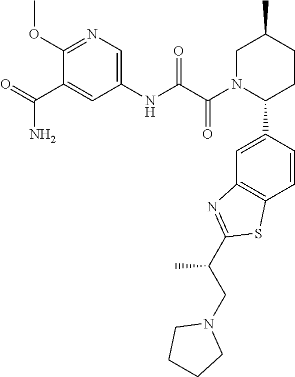 [SMILES: COc1ncc(NC(=O)C(=O)N2C[C@@H](C)CC[C@@H]2c2ccc3sc([C@@H](C)CN4CCCC4)nc3c2)cc1C(N)=O] | (abs) core, (or) sidechain | 62 | c | aa | aaa | A | A* |
| 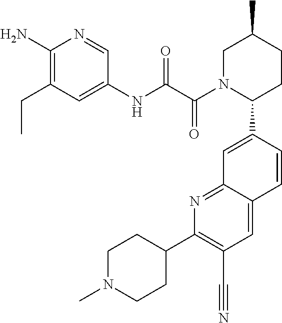 [SMILES: CCc1cc(NC(=O)C(=O)N2C[C@@H](C)CC[C@@H]2c2ccc3cc(C#N)c(C4CCN(C)CC4)nc3c2)cnc1N] | (abs) | 63 | c | aa | aaa | A | A* |
| 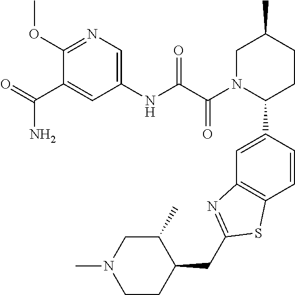 [SMILES: COc1ncc(NC(=O)C(=O)N2C[C@@H](C)CC[C@@H]2c2ccc3sc(C[C@H]4CCN(C)C[C@@H]4C)nc3c2)cc1C(N)=O] | (abs) core, (and) sidechain | 64 | c | aa | aaa | A | A* |
| 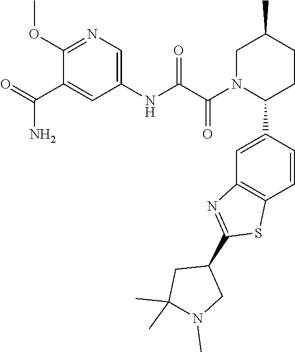 [SMILES: COc1ncc(NC(=O)C(=O)N2C[C@@H](C)CC[C@@H]2c2ccc3sc([C@H]4CN(C)C(C)(C)C4)nc3c2)cc1C(N)=O] | (abs) core, (or) sidechain | 65 | c | aa | aaa | B | A* |
| 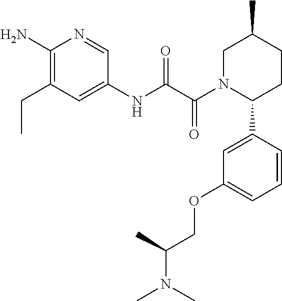 [SMILES: CCc1cc(NC(=O)C(=O)N2C[C@@H](C)CC[C@@H]2c2cccc(OC[C@H](C)N(C)C)c2)cnc1N] | (abs) | 66 | c | aa | aaa | A | A* |
| 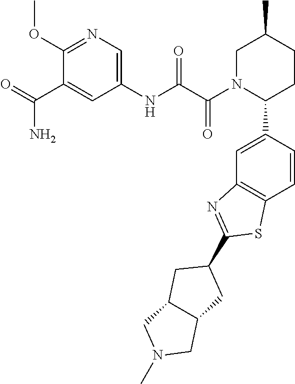 [SMILES: COc1ncc(NC(=O)C(=O)N2C[C@@H](C)CC[C@@H]2c2ccc3sc([C@H]4C[C@@H]5CN(C)C[C@@H]5C4)nc3c2)cc1C(N)=O] | (abs) core, (or) sidechain | 67 | c | aa | aaa | A | A* |
| 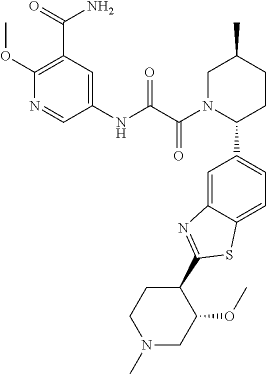 [SMILES: COc1ncc(NC(=O)C(=O)N2C[C@@H](C)CC[C@@H]2c2ccc3sc([C@@H]4CCN(C)C[C@H]4OC)nc3c2)cc1C(N)=O] | (abs) core, (or) sidechain | 68 | c | aa | aaa | A | A* |
| 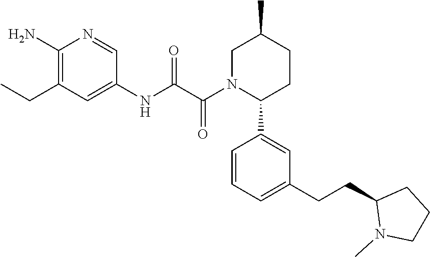 [SMILES: CCc1cc(NC(=O)C(=O)N2C[C@@H](C)CC[C@@H]2c2cccc(CC[C@H]3CCCN3C)c2)cnc1N] | (abs) | 69 | c | aa | aaa | A | A* |
| 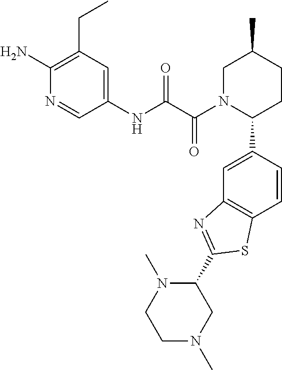 [SMILES: CCc1cc(NC(=O)C(=O)N2C[C@@H](C)CC[C@@H]2c2ccc3sc([C@@H]4CN(C)CCN4C)nc3c2)cnc1N] | (abs) core, (and) sidechain | 70 | c | bb | aaa | B | A* |
| 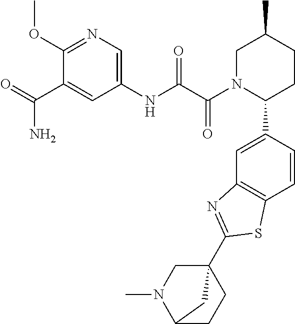 [SMILES: COc1ncc(NC(=O)C(=O)N2C[C@@H](C)CC[C@@H]2c2ccc3sc([C@]45CCC(C4)N(C)C5)nc3c2)cc1C(N)=O] | (abs) core, (or) sidechain | 71 | c | aa | aaa | B | A* |
| 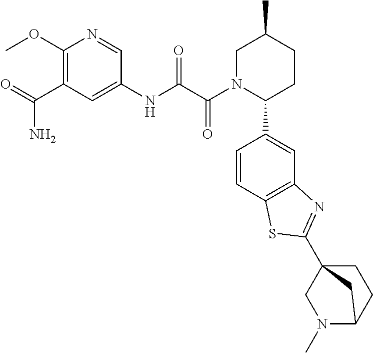 [SMILES: COc1ncc(NC(=O)C(=O)N2C[C@@H](C)CC[C@@H]2c2ccc3sc([C@@]45CCC(C4)N(C)C5)nc3c2)cc1C(N)=O] | (abs) core, (or) sidechain | Ent-71 | | | | | |
| 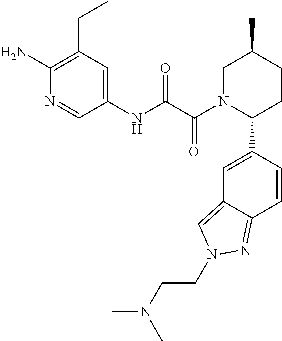 [SMILES: CCc1cc(NC(=O)C(=O)N2C[C@@H](C)CC[C@@H]2c2ccc3nn(CCN(C)C)cc3c2)cnc1N] | (abs) | 72 | c | bb | ccc | A | A* |
| 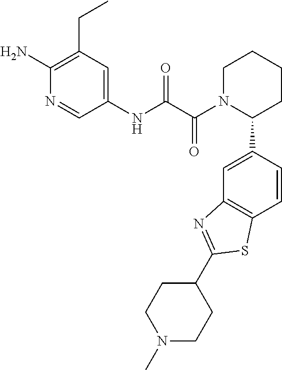 [SMILES: CCc1cc(NC(=O)C(=O)N2CCCC[C@@H]2c2ccc3sc(C4CCN(C)CC4)nc3c2)cnc1N] | (abs) | 73 | c | bb | bbb | A | B* |
| 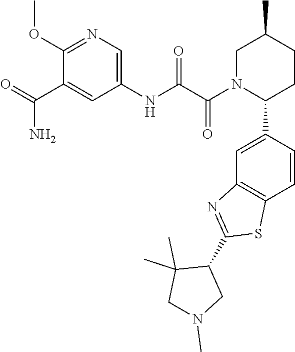 [SMILES: COc1ncc(NC(=O)C(=O)N2C[C@@H](C)CC[C@@H]2c2ccc3sc([C@@H]4CN(C)CC4(C)C)nc3c2)cc1C(N)=O] | (abs) core, (or) sidechain | 74 | b | aa | aaa | B | A* |
| 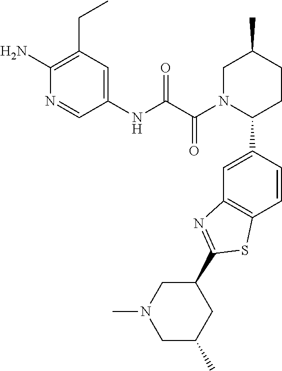 [SMILES: CCc1cc(NC(=O)C(=O)N2C[C@@H](C)CC[C@@H]2c2ccc3sc([C@H]4C[C@H](C)CN(C)C4)nc3c2)cnc1N] | (abs) core (or) sidechain | 75 | c | bb | aaa | B | A* |
| 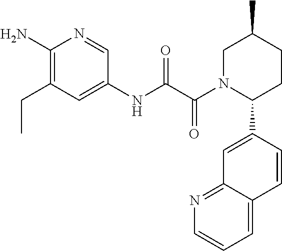 [SMILES: CCc1cc(NC(=O)C(=O)N2C[C@@H](C)CC[C@@H]2c2ccc3cccnc3c2)cnc1N] | (abs) | 76 | c | bb | ccc | A | B* |
| 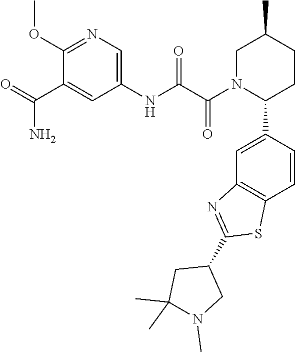 [SMILES: COc1ncc(NC(=O)C(=O)N2C[C@@H](C)CC[C@@H]2c2ccc3sc([C@@H]4CN(C)C(C)(C)C4)nc3c2)cc1C(N)=O] | (abs) core, (and) sidechain | 77 | c | aa | aaa | A | A* |
| 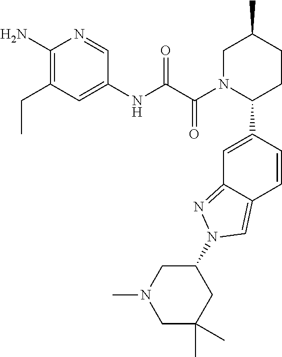 [SMILES: CCc1cc(NC(=O)C(=O)N2C[C@@H](C)CC[C@@H]2c2ccc3cn([C@H]4CN(C)CC(C)(C)C4)nc3c2)cnc1N] | (abs) core, (or) sidechain | 78 | c | aa | aaa | B | A* |
| 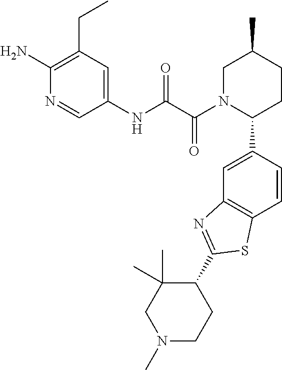 [SMILES: CCc1cc(NC(=O)C(=O)N2C[C@@H](C)CC[C@@H]2c2ccc3sc([C@@H]4CCN(C)CC4(C)C)nc3c2)cnc1N] | (abs) core, (and) sidechain | 79 | b | aa | aaa | C | A* |
| 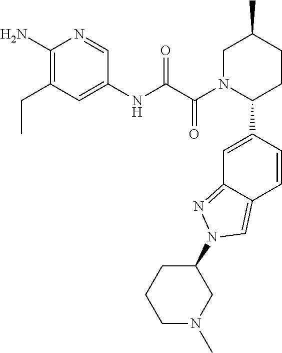 [SMILES: CCc1cc(NC(=O)C(=O)N2C[C@@H](C)CC[C@@H]2c2ccc3cn([C@@H]4CCCN(C)C4)nc3c2)cnc1N] | (abs) core, (or) sidechain | 80 | b | aa | aaa | C | A* |
| 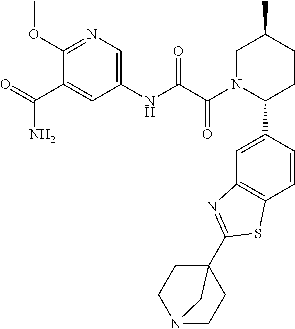 [SMILES: COc1ncc(NC(=O)C(=O)N2C[C@@H](C)CC[C@@H]2c2ccc3sc(C45CCN(CC4)C5)nc3c2)cc1C(N)=O] | (abs) | 81 | c | aa | aaa | A | A* |
| 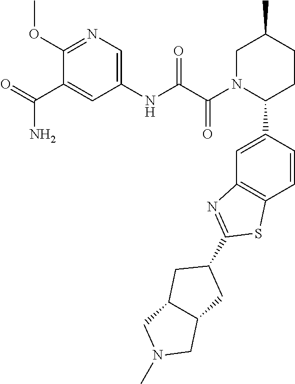 [SMILES: COc1ncc(NC(=O)C(=O)N2C[C@@H](C)CC[C@@H]2c2ccc3sc([C@@H]4C[C@@H]5CN(C)C[C@@H]5C4)nc3c2)cc1C(N)=O] | (abs) core, (or) sidechain | 82 | c | aa | aaa | A | A |
| 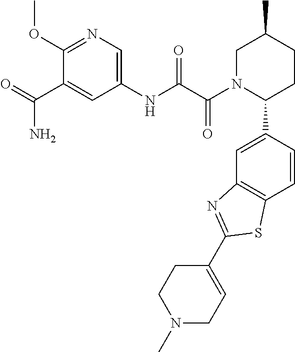 [SMILES: COc1ncc(NC(=O)C(=O)N2C[C@@H](C)CC[C@@H]2c2ccc3sc(C4=CCN(C)CC4)nc3c2)cc1C(N)=O] | (abs) | 83 | c | aa | aaa | A | A* |
| 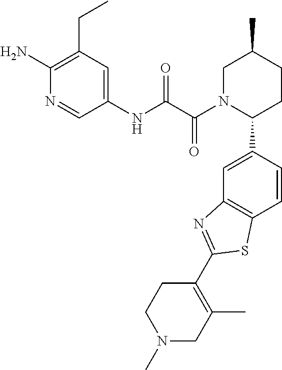 [SMILES: CCc1cc(NC(=O)C(=O)N2C[C@@H](C)CC[C@@H]2c2ccc3sc(C4=C(C)CN(C)CC4)nc3c2)cnc1N] | (abs) | 84 | c | aa | aaa | B | A* |
| 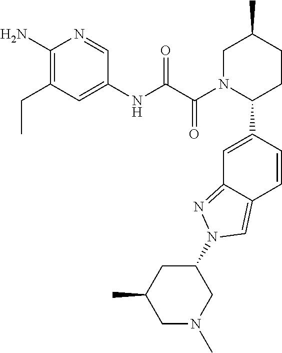 [SMILES: CCc1cc(NC(=O)C(=O)N2C[C@@H](C)CC[C@@H]2c2ccc3cn([C@H]4C[C@H](C)CN(C)C4)nc3c2)cnc1N] | (abs) core, (or) sidechain | 85 | c | aa | aaa | A | A* |
| 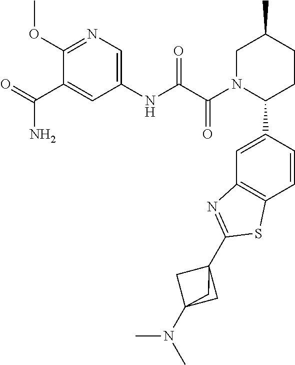 [SMILES: COc1ncc(NC(=O)C(=O)N2C[C@@H](C)CC[C@@H]2c2ccc3sc(C45CC(N(C)C)(C4)C5)nc3c2)cc1C(N)=O] | (abs) | 86 | c | bb | ccc | A | A* |
| 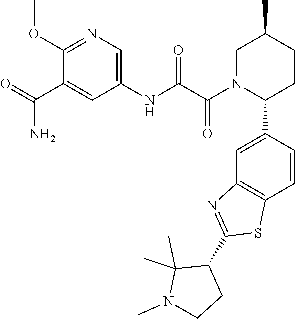 [SMILES: COc1ncc(NC(=O)C(=O)N2C[C@@H](C)CC[C@@H]2c2ccc3sc([C@@H]4CCN(C)C4(C)C)nc3c2)cc1C(N)=O] | (abs) core (and) sidechain | 87 | c | aa | aaa | A | A |
| 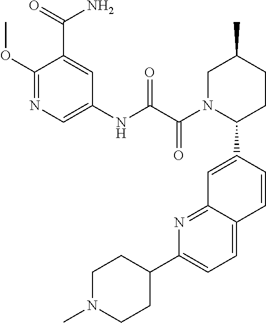 [SMILES: COc1ncc(NC(=O)C(=O)N2C[C@@H](C)CC[C@@H]2c2ccc3ccc(C4CCN(C)CC4)nc3c2)cc1C(N)=O] | (abs) | 88 | c | aa | aaa | A | A* |
| 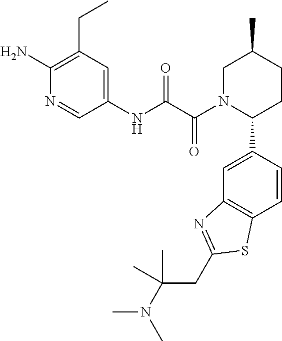 [SMILES: CCc1cc(NC(=O)C(=O)N2C[C@@H](C)CC[C@@H]2c2ccc3sc(CC(C)(C)N(C)C)nc3c2)cnc1N] | (abs) | 89 | c | aa | aaa | A | A* |
| 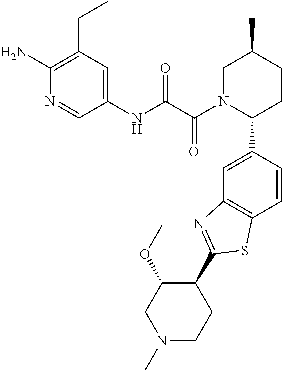 [SMILES: CCc1cc(NC(=O)C(=O)N2C[C@@H](C)CC[C@@H]2c2ccc3sc([C@H]4CCN(C)C[C@@H]4OC)nc3c2)cnc1N] | (abs) core, (or) sidechain | 90 | c | aa | aaa | A | A* |
| 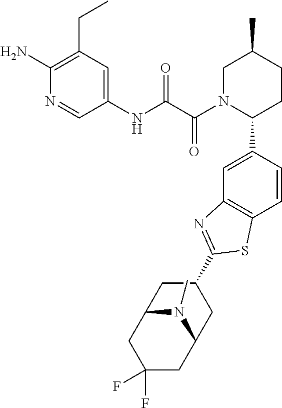 [SMILES: CCc1cc(NC(=O)C(=O)N2C[C@@H](C)CC[C@@H]2c2ccc3sc([C@@H]4C[C@@H]5CC(F)(F)C[C@H](C4)N5C)nc3c2)cnc1N] | (abs) core, (and) sidechain | 91 | c | aa | aaa | A | A* |
| 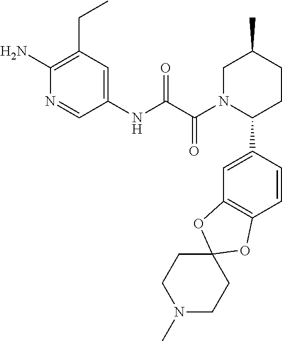 [SMILES: CCc1cc(NC(=O)C(=O)N2C[C@@H](C)CC[C@@H]2c2ccc3c(c2)OC2(CCN(C)CC2)O3)cnc1N] | (abs) | 92 | c | bb | bbb | A | A* |
| 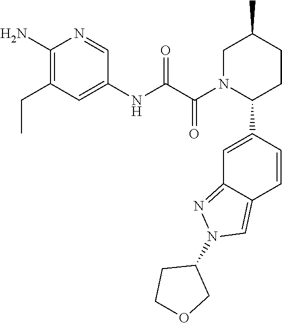 [SMILES: CCc1cc(NC(=O)C(=O)N2C[C@@H](C)CC[C@@H]2c2ccc3cn([C@H]4CCOC4)nc3c2)cnc1N] | (abs) core, (and) sidechain | 93 | c | bb | aaa | A | A |
| 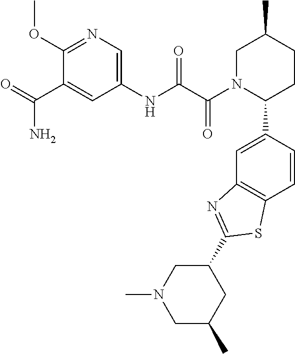 [SMILES: COc1ncc(NC(=O)C(=O)N2C[C@@H](C)CC[C@@H]2c2ccc3sc([C@@H]4C[C@@H](C)CN(C)C4)nc3c2)cc1C(N)=O] | (abs) core, (and) sidechain | 94 | c | aa | aaa | A | A* |
| 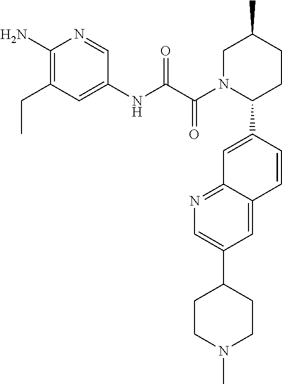 [SMILES: CCc1cc(NC(=O)C(=O)N2C[C@@H](C)CC[C@@H]2c2ccc3cc(C4CCN(C)CC4)cnc3c2)cnc1N] | (abs) | 95 | b | aa | aaa | B | A* |
| 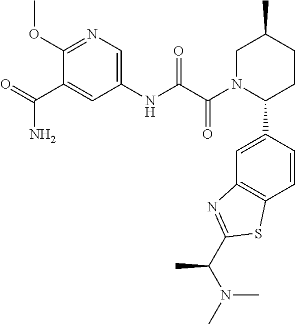 [SMILES: COc1ncc(NC(=O)C(=O)N2C[C@@H](C)CC[C@@H]2c2ccc3sc([C@H](C)N(C)C)nc3c2)cc1C(N)=O] | (abs) core, (or) sidechain | 96 | c | bb | bbb | A | A* |
| 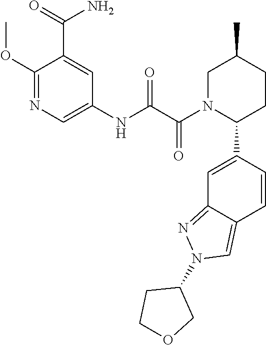 [SMILES: COc1ncc(NC(=O)C(=O)N2C[C@@H](C)CC[C@@H]2c2ccc3cn([C@H]4CCOC4)nc3c2)cc1C(N)=O] | (abs) core, (and) sidechain | 97 | c | bb | bbb | A | B* |
| 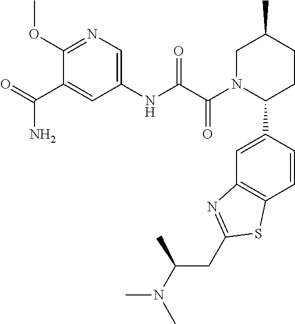 [SMILES: COc1ncc(NC(=O)C(=O)N2C[C@@H](C)CC[C@@H]2c2ccc3sc(C[C@H](C)N(C)C)nc3c2)cc1C(N)=O] | (abs) | 98 | c | aa | aaa | A | * |
| 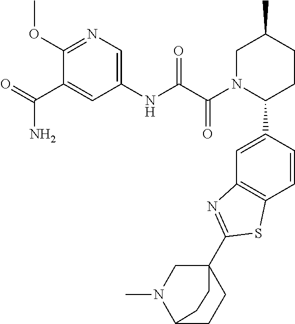 [SMILES: COc1ncc(NC(=O)C(=O)N2C[C@@H](C)CC[C@@H]2c2ccc3sc(C45CCC(CC4)N(C)C5)nc3c2)cc1C(N)=O] | (abs) | 99 | c | aa | aaa | A | A* |
| 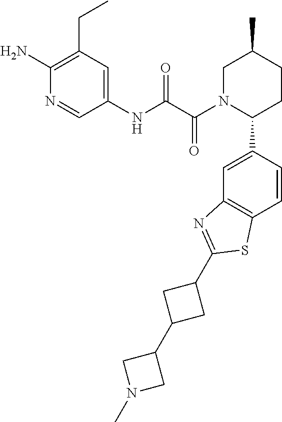 [SMILES: CCc1cc(NC(=O)C(=O)N2C[C@@H](C)CC[C@@H]2c2ccc3sc(C4CC(C5CN(C)C5)C4)nc3c2)cnc1N] | (abs) | 100 | b | aa | aaa | B | A* |
| 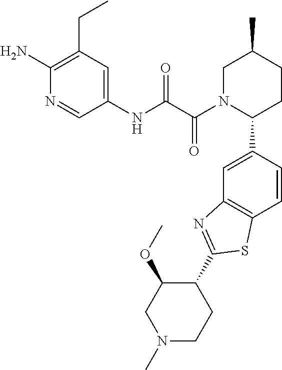 [SMILES: CCc1cc(NC(=O)C(=O)N2C[C@@H](C)CC[C@@H]2c2ccc3sc([C@@H]4CCN(C)C[C@H]4OC)nc3c2)cnc1N] | (abs) core, (or) sidechain | 101 | b | aa | aaa | B | A* |
| 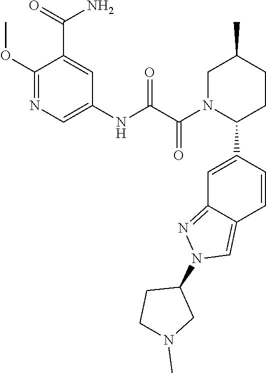 [SMILES: COc1ncc(NC(=O)C(=O)N2C[C@@H](C)CC[C@@H]2c2ccc3cn([C@@H]4CCN(C)C4)nc3c2)cc1C(N)=O] | (abs) | 102 | c | aa | aaa | A | A* |
| 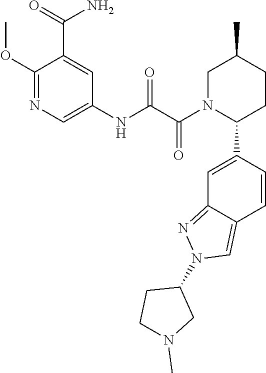 [SMILES: COc1ncc(NC(=O)C(=O)N2C[C@@H](C)CC[C@@H]2c2ccc3cn([C@H]4CCN(C)C4)nc3c2)cc1C(N)=O] | (abs) | 103 | c | aa | aaa | A | A* |
| 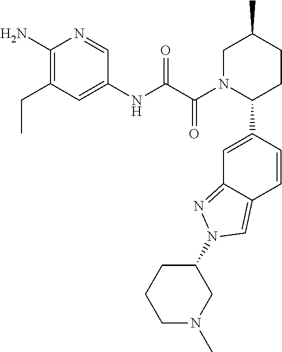 [SMILES: CCc1cc(NC(=O)C(=O)N2C[C@@H](C)CC[C@@H]2c2ccc3cn([C@H]4CCCN(C)C4)nc3c2)cnc1N] | (abs) core, (or) sidechain | 104 | c | aa | aaa | B | A* |
| 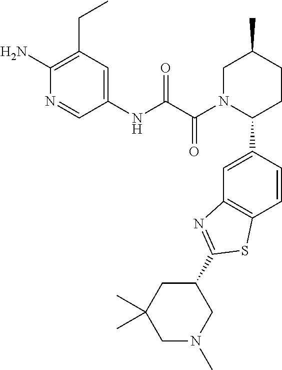 [SMILES: CCc1cc(NC(=O)C(=O)N2C[C@@H](C)CC[C@@H]2c2ccc3sc([C@@H]4CN(C)CC(C)(C)C4)nc3c2)cnc1N] | (abs) core, (or) sidechain | 105 | c | aa | aaa | A | A* |
| 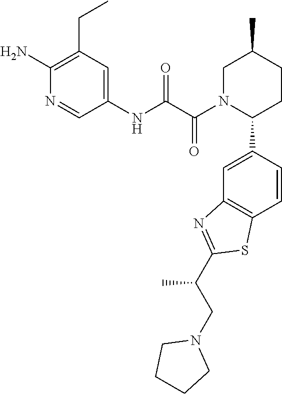 [SMILES: CCc1cc(NC(=O)C(=O)N2C[C@@H](C)CC[C@@H]2c2ccc3sc([C@@H](C)CN4CCCC4)nc3c2)cnc1N] | (abs) core, (or) sidechain | 106 | b | aa | aaa | B | A* |
| 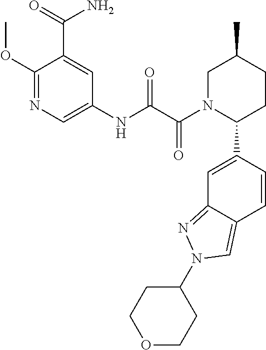 [SMILES: COc1ncc(NC(=O)C(=O)N2C[C@@H](C)CC[C@@H]2c2ccc3cn(C4CCOCC4)nc3c2)cc1C(N)=O] | (abs) | 107 | c | bb | bbb | A | B* |
| 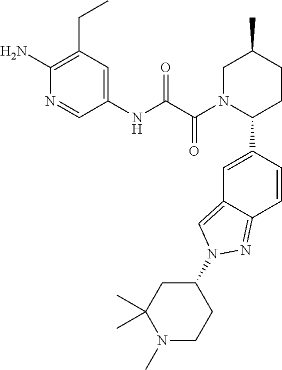 [SMILES: CCc1cc(NC(=O)C(=O)N2C[C@@H](C)CC[C@@H]2c2ccc3nn([C@@H]4CCN(C)C(C)(C)C4)cc3c2)cnc1N] | (abs) core, (or) sidechain | 108 | b | aa | aaa | B | A* |
| 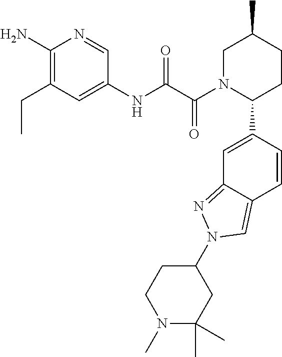 [SMILES: CCc1cc(NC(=O)C(=O)N2C[C@@H](C)CC[C@@H]2c2ccc3cn(C4CCN(C)C(C)(C)C4)nc3c2)cnc1N] | (abs) | 109 | b | aa | aaa | B | A* |
| 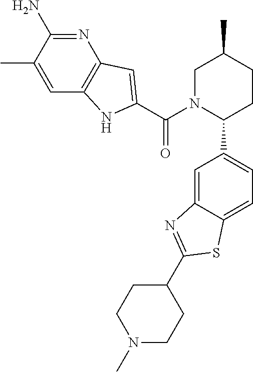 [SMILES: Cc1cc2[nH]c(C(=O)N3C[C@@H](C)CC[C@@H]3c3ccc4sc(C5CCN(C)CC5)nc4c3)cc2nc1N] | (abs) | 110 | c | aa | aaa | A | A* |
| 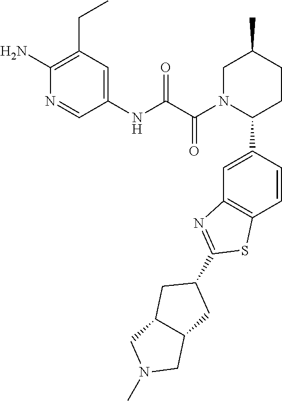 [SMILES: CCc1cc(NC(=O)C(=O)N2C[C@@H](C)CC[C@@H]2c2ccc3sc([C@@H]4C[C@@H]5CN(C)C[C@@H]5C4)nc3c2)cnc1N] | (abs) core, (or) sidechain | 111 | c | aa | aaa | B | A* |
| 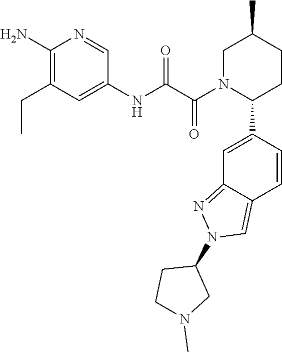 [SMILES: CCc1cc(NC(=O)C(=O)N2C[C@@H](C)CC[C@@H]2c2ccc3cn([C@@H]4CCN(C)C4)nc3c2)cnc1N] | (abs) | 112 | b | aa | aaa | B | A* |
| 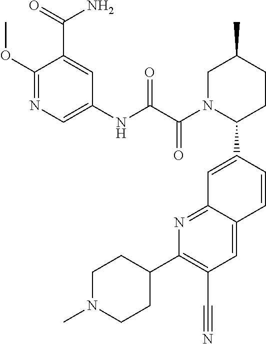 [SMILES: COc1ncc(NC(=O)C(=O)N2C[C@@H](C)CC[C@@H]2c2ccc3cc(C#N)c(C4CCN(C)CC4)nc3c2)cc1C(N)=O] | (abs) | 113 | c | aa | aaa | A | A* |
| 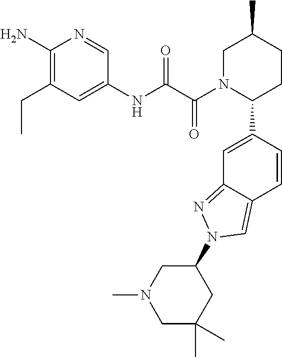 [SMILES: CCc1cc(NC(=O)C(=O)N2C[C@@H](C)CC[C@@H]2c2ccc3cn([C@@H]4CN(C)CC(C)(C)C4)nc3c2)cnc1N] | (abs) core (or) sidechain | 114 | b | aa | aaa | B | A* |
| 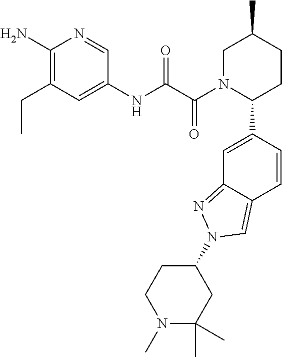 [SMILES: CCc1cc(NC(=O)C(=O)N2C[C@@H](C)CC[C@@H]2c2ccc3cn([C@H]4CCN(C)C(C)(C)C4)nc3c2)cnc1N] | (abs) core, (or) sidechain | 115 | b | aa | aaa | 0 | A* |
| 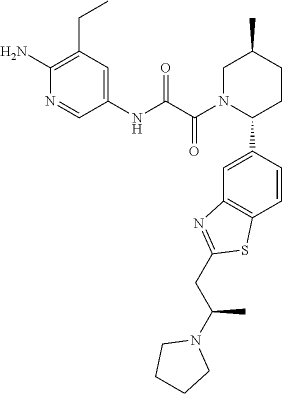 [SMILES: CCc1cc(NC(=O)C(=O)N2C[C@@H](C)CC[C@@H]2c2ccc3sc(C[C@@H](C)N4CCCC4)nc3c2)cnc1N] | (abs) core, (and) sidechain | 116 | c | aa | aaa | A | A* |
| 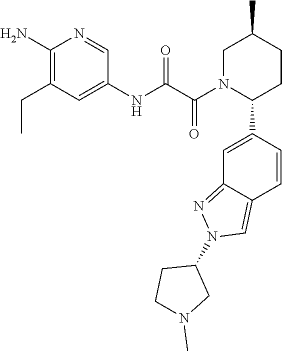 [SMILES: CCc1cc(NC(=O)C(=O)N2C[C@@H](C)CC[C@@H]2c2ccc3cn([C@H]4CCN(C)C4)nc3c2)cnc1N] | (abs) | 117 | c | bb | aaa | C | A* |
| 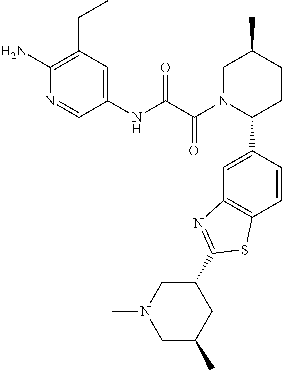 [SMILES: CCc1cc(NC(=O)C(=O)N2C[C@@H](C)CC[C@@H]2c2ccc3sc([C@@H]4C[C@@H](C)CN(C)C4)nc3c2)cnc1N] | (abs) core, (and) sidechain | 118 | b | aa | aaa | C | A* |
| 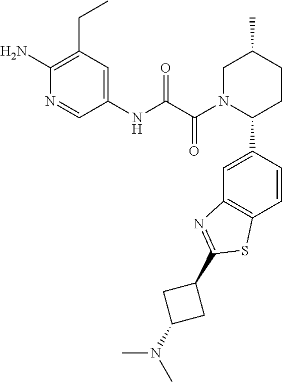 [SMILES: CCc1cc(NC(=O)C(=O)N2C[C@H](C)CC[C@@H]2c2ccc3sc([C@H]4C[C@H](N(C)C)C4)nc3c2)cnc1N] | (abs) core, (and) sidechain | 119 | c | cc | ccc | C | C* |
| 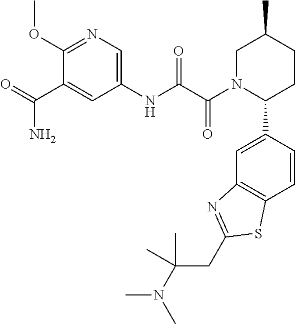 [SMILES: COc1ncc(NC(=O)C(=O)N2C[C@@H](C)CC[C@@H]2c2ccc3sc(CC(C)(C)N(C)C)nc3c2)cc1C(N)=O] | (abs) | 120 | c | aa | aaa | A | A* |
| 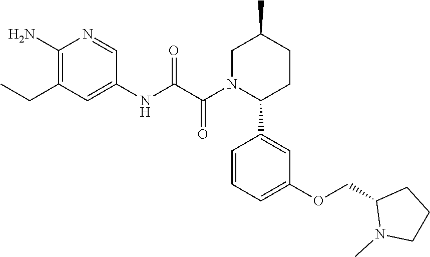 [SMILES: CCc1cc(NC(=O)C(=O)N2C[C@@H](C)CC[C@@H]2c2cccc(OC[C@@H]3CCCN3C)c2)cnc1N] | (abs) core, (or) sidechain | 121 | c | bb | aaa | B | A* |
| 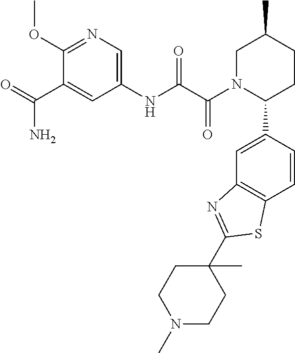 [SMILES: COc1ncc(NC(=O)C(=O)N2C[C@@H](C)CC[C@@H]2c2ccc3sc(C4(C)CCN(C)CC4)nc3c2)cc1C(N)=O] | (abs) | 122 | c | aa | aaa | A | A* |
| 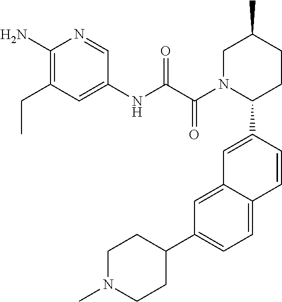 [SMILES: CCc1cc(NC(=O)C(=O)N2C[C@@H](C)CC[C@@H]2c2ccc3ccc(C4CCN(C)CC4)cc3c2)cnc1N] | (abs) | 123 | c | aa | aaa | A | A* |
| 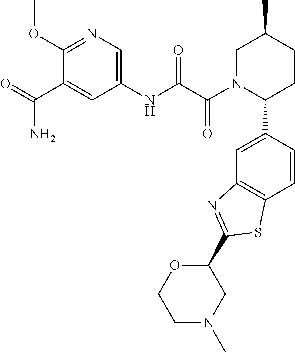 [SMILES: COc1ncc(NC(=O)C(=O)N2C[C@@H](C)CC[C@@H]2c2ccc3sc([C@H]4CN(C)CCO4)nc3c2)cc1C(N)=O] | (abs) core, (and) sidechain | 124 | c | bb | ccc | A | B* |
| 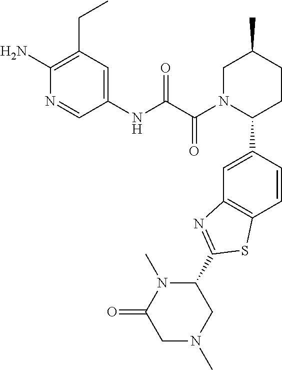 [SMILES: CCc1cc(NC(=O)C(=O)N2C[C@@H](C)CC[C@@H]2c2ccc3sc([C@@H]4CN(C)CC(=O)N4C)nc3c2)cnc1N] | (abs) core, (and) sidechain | 125 | c | bb | bbb | B | A* |
| 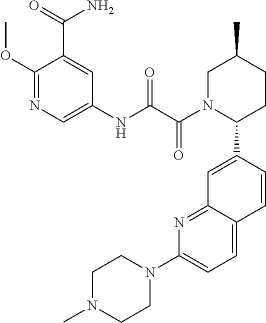 [SMILES: COc1ncc(NC(=O)C(=O)N2C[C@@H](C)CC[C@@H]2c2ccc3ccc(N4CCN(C)CC4)nc3c2)cc1C(N)=O] | (abs) | 126 | c | aa | aaa | A | A* |
| 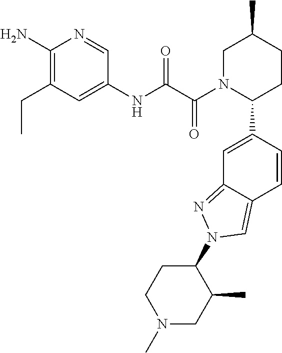 [SMILES: CCc1cc(NC(=O)C(=O)N2C[C@@H](C)CC[C@@H]2c2ccc3cn([C@@H]4CCN(C)C[C@@H]4C)nc3c2)cnc1N] | (abs) core, (or) sidechain | 127 | b | aa | aaa | C | A* |
| 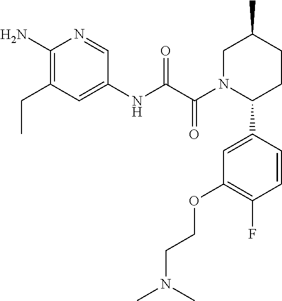 [SMILES: CCc1cc(NC(=O)C(=O)N2C[C@@H](C)CC[C@@H]2c2ccc(F)c(OCCN(C)C)c2)cnc1N] | (abs) | 128 | c | bb | ccc | A | A* |
| 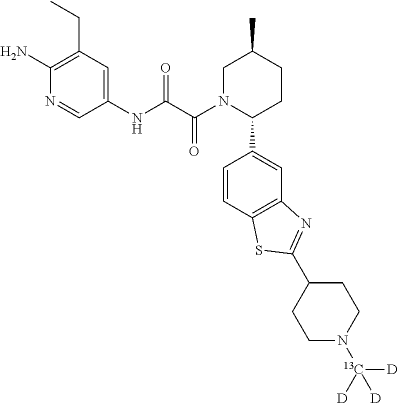 [SMILES: [2H][13C]([2H])([2H])N1CCC(c2nc3cc([C@H]4CC[C@H](C)CN4C(=O)C(=O)Nc4cnc(N)c(CC)c4)ccc3s2)CC1] | (abs) | 129 | b | aa | aaa | C | A* |

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%0, at least 10%, at least 1500, at least 20%, at least 25%, at least 30%, at least 35%, at least 40%, at least 45%, at least 5000, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In another embodiment, provided is a pharmaceutical composition comprising a pharmaceutically acceptable carrier and an effective amount of a compound described herein (e.g., a compound of Table 1), or a pharmaceutically acceptable salt thereof.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions provided herewith include, but are not limited to, ion exchangers, alumina, aluminum stearate, lecithin, self emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat. Cyclodextrins such as α-, β-, and γ-cyclodextrin, or chemically modified derivatives such as hydroxyalkylcyclodextrins, including 2 and 3 hydroxypropyl-β-cyclodextrins, or other solubilized derivatives may also be advantageously used to enhance delivery of compounds of the formulae described herein.

When employed as pharmaceuticals, the compounds provided herein are typically administered in the form of a pharmaceutical composition. Such compositions can be prepared in a manner well known in the pharmaceutical art and comprise at least one active compound.

In one embodiment, with respect to the pharmaceutical composition, the carrier is a parenteral carrier, oral or topical carrier.

Also provided is a compound described herein (e.g., a compound of Table 1, or pharmaceutically acceptable salts thereof) (or pharmaceutical composition thereof) for use as a pharmaceutical or a medicament (e.g., a medicament for the treatment of an MTAP-deficient and/or an MTA-accumulating disease in a subject in need thereof). In one embodiment, the disease is a proliferating disease. In a further embodiment, the disease is an MTAP-deficient and/or MTA-accumulating cancer. In one embodiment, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Also provided is a compound described herein (e.g., a compound of Table 1, or pharmaceutically acceptable salts thereof) (or pharmaceutical composition thereof) for use in the treatment of an MTAP-deficient and/or an MTA-accumulating disease in a subject in need thereof. In one embodiment, the disease is a proliferating disease. In a further embodiment, the disease is an MTAP-deficient and/or MTA-accumulating cancer. In one embodiment, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Also provided is a compound described herein (e.g., a compound of Table 1, or pharmaceutically acceptable salts thereof) (or pharmaceutical composition thereof) for use in the manufacturing of a medicament (e.g., a medicament for the treatment of an MTAP-deficient and/or an MTA-accumulating disease in a subject in need thereof). In one embodiment, the disease is a proliferating disease. In a further embodiment, the disease is an MTAP-deficient and/or MTA-accumulating cancer. In one embodiment, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Generally, the compounds provided herein are administered in an effective amount (e.g., a therapeutically effective amount). The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional nontoxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The pharmaceutical compositions provided herewith may also be administered in the form of suppositories for rectal administration. These compositions can be prepared by mixing a compound provided herewith with a suitable non irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions provided herewith may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The above-described components for orally administrable, injectable or topically administrable, rectally administrable and nasally administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds described herein can also be administered in sustained release forms or from sustained release drug delivery systems. A description of representative sustained release materials can be found in Remington's Pharmaceutical Sciences.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

Also provided is the pharmaceutically acceptable acid addition salt of a compound described herein (e.g., a compound of Table 1).

The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

### Methods of Treatment and Use

Treatment of MTAP-Deficient and/or MTA-Accumulating Proliferation Disorders

5-Methylthioadenosine phosphorylase (MTAP) catalyzes the reversible phosphorylation of S-methyl-5′-thioadenosine (MTA) to adenine and 5-methylthioribose-1-phosphate. MTAP-deletion is a common genetic event in human cancer. MTAP deletion frequency in a subset of human cancers is described in Cerami et al., Cancer Discov. (2012); 2(5):401-4; Gao et al., Sci Signal. (2013); 6(269):pl1; and Lee et al., Nat. Gen. (2014) 46(11):1227-32. For example, more than 50% of malignant peripheral nerve sheath tumor (MPNST) have deletions in MTAP (Lee et al., Nat. Gen. (2014)). Other cancers with high MTAP deletion frequencies are glioblastoma (GBM), mesothelioma, bladder cancer, pancreatic cancer, esophageal cancer, squamous lung cancer, melanoma, diffuse large B cell lymphoma (DLBCL), head and neck cancer, cholangiocarcinoma, lung adenoma, sarcoma, stomach cancer, glioma, adrenal carcinoma, thymoma, breast cancer, liver cancer, ovarian cancer, renal papillary cancer, uterine cancer, prostate cancer, and renal clear cell cancer. MTAP deletion in cells is one of the mechanisms that leads to MTAP-deficiency, increased intracellular MTA accumulation, and confers enhanced dependency on the protein arginine methyltransferase 5 (PRMT5) in cancer cells. Other mechanisms leading to MTAP deficiency include, inter alia, MTAP translocations and MTAP epigenetic silencing which could also lead to MTAP-null and/or MTAP deficient tumors. PRMT5 mediates the formation of symmetric dimethylarginine (SDMA); thus, the PRMT5 activity can be assessed by measuring the SDMA levels using the antibody against an SDMA or SDMA modified polypeptide.

In one embodiment, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of a compound of the present disclosure (e.g., a compound of Table 1) or a pharmaceutically acceptable salt thereof.

In some embodiments, provided is a compound of the present disclosure (e.g., a compound of Table 1), or a pharmaceutical composition comprising a compound of the present disclosure for use in a method of treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a compound of the present disclosure (e.g., a compound of Table 1), or a pharmaceutical composition comprising a compound of the present disclosure for use in the manufacturing of a medicament for treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a use of a compound of the present disclosure (e.g., a compound of Table 1), or of a pharmaceutical composition comprising a compound of the present disclosure in a method of treating human or animal subjects having or having been diagnosed with an MTAP-deficiency-related and/or MTA-accumulating proliferative disorder (e.g., cancer). In some embodiments, the use is of a therapeutically effective amount of the compound or composition.

In some embodiments, provided is use of a compound of the present disclosure (e.g., a compound of Table 1), or of a pharmaceutical composition comprising a compound of the present disclosure in the manufacturing of a medicament for treating human or animal subjects having or having been diagnosed with an MTAP-deficiency-related and/or MTA-accumulating proliferative disorder (e.g., cancer). In some embodiments, the use is of a therapeutically effective amount of the compound or composition.

In one embodiment, provided are methods for treating an MTAP-deficiency-related and/or MTA-accumulating proliferative disorder (e.g., cancer) in a subject in need thereof comprising administering to the subject an effective amount (e.g., a therapeutically effective amount) of a compound of the present disclosure (e.g., a compound of Table 1) or a pharmaceutically acceptable salt thereof.

In one embodiment, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of pharmaceutical composition of the present disclosure (e.g., a composition comprising a compound of Table 1, or pharmaceutically acceptable salts thereof and a pharmaceutically acceptable carrier). In one embodiment, the compound or composition is administered in combination with a second therapeutic agent.

In one embodiment, provided are methods of treating an MTAP-deficiency-related and/or MTA-accumulating proliferative disorder (e.g., cancer) in a subject in need thereof comprising administering to the subject a therapeutically effective amount of pharmaceutical composition of the present disclosure (e.g., a composition comprising a compound of Table 1, or pharmaceutically acceptable salts thereof and a pharmaceutically acceptable carrier). In one embodiment, the compound or composition is administered in combination with a second therapeutic agent.

In some embodiments, the subject is human.

In certain embodiments, the disease is an MTAP-deficient and/or MTA-accumulating cancer.

In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment, the cancer is an MTAP-deficient and/or MTA-accumulating glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive or mixed mode PRMT5 inhibitor or an MTA cooperative binding agent, e.g., a compound of Table 1, or pharmaceutically acceptable salts thereof) described herein can be used in a method of inhibiting proliferation of MTAP-deficient cells in a subject in need thereof, the method comprising the step of administering to the subject, a PRMT5 inhibitor (e.g., an MTA-uncompetitive, non-competitive or mixed mode PRMT5 inhibitor or an MTA cooperative binding agent, e.g., a compound of Table 1, or pharmaceutically acceptable salts thereof) in an amount that is effective to inhibit proliferation of the MTAP-deficient cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive or mixed mode PRMT5 inhibitor or an MTA cooperative binding agent, e.g., a compound of Table 1, or pharmaceutically acceptable salts thereof) described herein can be used in a method of inhibiting proliferation of MTA-accumulating cells in a subject in need thereof, the method comprising the step of administering to the subject, a PRMT5 inhibitor (e.g., an MTA-uncompetitive, non-competitive or mixed mode PRMT5 inhibitor or an MTA cooperative binding agent, e.g., a compound of Table 1, or pharmaceutically acceptable salts thereof) in an amount that is effective to inhibit proliferation of the MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive, or mixed mode PRMT5 inhibitor or an MTA-cooperative binding agent, e.g., a compound of Table 1, or pharmaceutically acceptable salts thereof) described herein can be used in a method of inhibiting proliferation of MTAP deficient and/or MTA-accumulating cells in a subject in need thereof, the method comprising the step of administering to the subject, a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof) in an amount that is effective to inhibit proliferation of the MTAP deficient and/or MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

### Combination Therapies

In some embodiments, provided are methods of treatment of MTAP-deficient and/or MTA accumulating proliferative disorders (e.g., cancers) with a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof) in combination with one or more therapeutic agent.

In some embodiments, provided are methods of treatment of MTAP-deficient and/or MTA accumulating proliferative disorders (e.g., cancers) with a PRMT5 inhibitor described herein (e.g., a compound of Table 1, or pharmaceutically acceptable salts thereof) in combination with a second therapeutic agent. In some embodiments, provided are methods of treatment of MTAP-deficient and/or MTA accumulating proliferative disorders (e.g., cancers) with PRMT5 inhibitor described herein (e.g., a compound of Table 1, or pharmaceutically acceptable salts thereof) in combination with a second therapeutic agent and a third therapeutic agent. In some embodiments, provided are methods of treatment of MTAP-deficient and/or MTA accumulating proliferative disorders (e.g., cancers) with PRMT5 inhibitor described herein (e.g., a compound of Table 1, or pharmaceutically acceptable salts thereof) in combination with a second therapeutic agent, a third therapeutic agent, and a fourth therapeutic agent.

The term “Combination” refers to either a fixed combination in one dosage unit form, or a combined administration where a PRMT5 inhibitor described herein (e.g., a compound of Table 1, or pharmaceutically acceptable salts thereof) and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., PRMT5 inhibitors described herein (e.g., a compound of Table 1, or pharmaceutically acceptable salts thereof) and a combination partner, are both administered to a patient simultaneously in the form of a single entity or dosage. The term “non-fixed combination” means that the therapeutic agents, e.g., a PRMT5 inhibitor described herein (e.g., a compound of Table 1, or pharmaceutically acceptable salts thereof) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In certain embodiments, PRMT5 inhibitors described herein are combined with other therapeutic agents, including, but not limited to, other anti-cancer agents, anti-allergic agents, anti-nausea agents (or anti-emetics), pain relievers, cytoprotective agents, and combinations thereof.

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and a general chemotherapeutic agents selected from anastrozole (Arimidex®), bicalutamide (Casodex®), bleomycin sulfate (Blenoxane®), busulfan (Myleran®), busulfan injection (Busulfex®), capecitabine (Xeloda®), N₄-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and an EGFR-inhibitor (e.g., cetuximab, panitumimab, erlotinib, gefitinib and EGFRi NOS). In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and a MAPK-pathway inhibitor (e.g., BRAFi, panRAFi, MEKi, ERKi). In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and a PI3K-mTOR pathway inhibitor (e.g., alpha-specific PI3Ki, pan-class I PI3Ki and mTOR/PI3Ki, particularly everolimus and analogues thereof).

MTAP-deletion can co-occur with mutations in the KRAS gene (e.g., KRASG12C). In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof), and a KRAS inhibitor (e.g., a pan-KRAS or a specific G12C, G12D, G13C inhibitor, e.g., adagrasib, sotorasib, LY3537982, RMC-6236, RMC-6291, RMC-9805, RMC-8839).

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof), and a Spliceosome inhibitor (e.g., SF3b1 inhibitors; e.g., E7107).

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and an HDAC inhibitor or DNA methyltransferase inhibitor. In some embodiments, the HDAC inhibitor is Trichostatin A. In some embodiments, the DNA methyltransferase inhibitor is 5-azacytidine.

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and a MAT2A inhibitor.

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and an inhibitor of a protein which interacts with or is required for PRMT5 function, including, but not limited to, pICIN, WDR77 or RIOK1.

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and an HDM2 inhibitor and/or 5-FU or other purine analogues (e.g., 6-thioguanine, 6-mercaptopurine).

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and a CDK4 inhibitor, including, but not limited to, LEE011 or a CDK 4/6 inhibitor (e.g., palbociclib (Ibrance®), ribociclib (Kisqali®), and abemaciclib (Verzenio®).

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and a targeted treatment contingent on the dependency of individual target tumors on relevant pathways as determined by suitable predictive markers, including but not limited to: inhibitors of HDM2i, PI3K/mTOR-I, MAPKi, RTKi (EGFRi, FGFRi, METi, IGFiRi, JAKi, and WNTi.

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and immunotherapy.

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and an immunotherapeutic agent.

In some embodiments, the immunotherapeutic agent is an anti-CTLA-4 antibody (e.g., ipilimumab, tremelimumab).

In some embodiments, the immunotherapeutic agent is an anti-PD-1 or anti-PD-L1 agent (e.g., an antibody). In some embodiments, the immunotherapeutic agent is an anti-PD-1 agent (e.g., an anti-PD-1 antibody, e.g., nivolumab (i.e., MDX-1106, BMS-936558, ONO-4538); CT-011; AMP-224; pembrolizumab (MK-3475); pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab). In some embodiments, the immunotherapeutic agent is an anti-PD-L1 agent (e.g., an anti-PD-L1 antibody, e.g., BMS936559 (i.e., MDX-1105); durvalumab (MED14736); avelumab (MSB0010718C); envafolimab; cosibelimab; sugemalimab, AUNP-12 or atezolizumab (MPDL-3280A) or an anti-PD-L1 small molecule (e.g., CA-170)).

In some embodiments, the immunotherapeutic agent is a checkpoint blocking antibody (e.g., anti-TIM3, anti-LAG3, anti-TIGIT including IMP321 and MGA271).

In some embodiments, the immunotherapeutic agent is a cell-based therapy. In some embodiments, the cell-based therapy is a CAR-T therapy.

In some embodiments, the immunotherapeutic agent is a co-stimulatory antibody (e.g., anti-4-1BB, anti-OX40, anti-GITR, anti-CD27, anti-CD40).

In some embodiments, the immunotherapeutic agent is a cancer vaccine such as a neoantigen. These vaccines can be developed using peptides or RNA (e.g., mRNA).

In some embodiments, the immunotherapeutic agent is an oncolytic virus.

In some embodiments, the immunotherapeutic agent is a STING pathway agonist. Exemplary STING agonists include MK-1454 and ADU-S100.

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and a disease-specific huMAB (e.g., an anti-HER3 huMAB).

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and an ADC/ADCC contingent on the expression of relevant surface targets on target tumors of interest.

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and one or more DNA damage pathway inhibitor.

In some embodiments, a DNA damage pathway inhibitor is selected from the group consisting of bleomycin, an ATM inhibitor (e.g., AZD1390), a USP1 inhibitor, a WEE1 inhibitor (e.g., AZD1775), and a Chk1 inhibitor (e.g., AZD7762). In some embodiments, a DNA damage pathway inhibitor is a DNA alkylating agent.

In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and a PARP inhibitor. In some embodiments, a PARP inhibitor is selected from the group consisting of olaparib, rucaparib, niraparib, talazoparib, veliparib, pamiparib, CEP 9722, E7016, iniparib, and 3-aminobenzamide.

Some patients may experience allergic reactions to the PRMT5 inhibitors described herein and/or other anti-cancer agent(s) during or after administration; therefore, anti-allergic agents are often administered to minimize the risk of an allergic reaction. In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and an anti-allergic agent (e.g., a corticosteroid, including, but not limited to, dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); an antihistamine, such as diphenhydramine (e.g., Benadryl®), hydroxyzine, and cyproheptadine; a bronchodilator, such as the beta-adrenergic receptor agonists, albuterol (e.g., Proventil®), and terbutaline (Brethine®)).

Some patients may experience nausea during and after administration of the PRMT5 inhibitors described herein and/or other anti-cancer agent(s); therefore, anti-emetics are used in preventing nausea (upper stomach) and vomiting. In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and an anti-emetic (e.g., aprepitant (Emend®), ondansetron (Zofran®), granisetron HCl (Kytril®), lorazepam (Ativan®), dexamethasone (Decadron®), prochlorperazine (Compazine®), casopitant (Rezonic® and Zunrisa®), and combinations thereof).

Medication to alleviate the pain experienced during the treatment period is often prescribed to make the patient more comfortable. In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and an analgesic (e.g., an over-the-counter analgesic (e.g., Tylenol®), an opioid analgesic (e.g., hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), fentanyl (e.g., Duragesic®))).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and a cytoprotective agent (e.g., Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid)).

The structure of the active compounds identified by code numbers, generic or trade names may be taken from the actual edition of the standard compendium “The Merck Index” or from databases, e.g., Patents International (e.g., IMS World Publications).

The above-mentioned compounds, which can be used in combination with a PRMT5 inhibitor as described herein, can be prepared and administered as described in the art, including, but not limited to, in the documents cited above.

In one embodiment, provided are pharmaceutical compositions comprising at least one compound of the present disclosure (e.g., a PRMT5 inhibitor, e.g., a compound of Table 1) or a pharmaceutically acceptable salt thereof together with a pharmaceutically acceptable carrier suitable for administration to a human or animal subject, either alone or together with other anti-cancer agents.

In one embodiment, provided are methods of treating human or animal subjects having or having been diagnosed with an MTAP-deficient and/or MTA accumulating proliferative disorder (e.g., cancer) comprising administering to the subject in need thereof a pharmaceutical composition comprising a therapeutically effective amount of a compound of the present disclosure (e.g., a compound of Table 1) or a pharmaceutically acceptable salt thereof in combination with one or more therapeutic agents as described herein.

In one embodiment, provided are methods of treating an MTAP-deficient and/or MTA accumulating proliferative disorder (e.g., cancer) in a subject in need thereof comprising administering to the subject a pharmaceutical composition comprising an effective amount (e.g., a therapeutically effective amount) of a compound of the present disclosure (e.g., a compound of Table 1) or a pharmaceutically acceptable salt thereof in combination with one or more therapeutic agents as described herein.

In particular, compositions will either be formulated together as a combination therapeutic or administered separately.

In combination therapy, a PRMT5 inhibitor as described herein and other anti-cancer agent(s) may be administered either simultaneously, concurrently or sequentially with no specific time limits, wherein such administration provides therapeutically effective levels of the two compounds in the body of the patient.

In a preferred embodiment, the compound of the present disclosure (e.g., a compound of Table 1, or pharmaceutically acceptable salts thereof) and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The PRMT5 inhibitor as described herein and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another embodiment, provided are kits that include one or more PRMT5 inhibitor(s) as described herein (e.g., a compound of Table 1, or pharmaceutically acceptable salts thereof) and a second therapeutic agent as disclosed herein are provided. Representative kits include (a) a PRMT5 inhibitor as described herein or a pharmaceutically acceptable salt thereof (e.g., a compound of Table 1, or pharmaceutically acceptable salts thereof), (b) at least one other therapeutic agent, e.g., as indicated above, whereby such kit may comprise a package insert or other labeling including directions for administration.

A PRMT5 inhibitor as described herein (e.g., a compound of Table 1, or pharmaceutically acceptable salts thereof) may also be used in combination with known therapeutic processes, for example, the administration of hormones or especially radiation. A compound of the present disclosure may in particular be used as a radiosensitizer, especially for the treatment of tumors which exhibit poor sensitivity to radiotherapy. In some embodiments, provided is a method of treating a disease or disorder (e.g., cancer) comprising administering or coadministering, in any order, to a patient in need thereof a PRMT5 inhibitor described herein (e.g., a compound of Table 1, or pharmaceutically acceptable salts thereof) and radiation.

### Patient Selection and Monitoring

In one embodiment, provided is a method of determining if a subject having or having been diagnosed with a cancer (e.g., a cancer patient) will respond to therapeutic treatment with a PRMT5 inhibitor (e.g., an MTA-uncompetitive PRMT5 inhibitor, e.g., a compound of Table 1, or pharmaceutically acceptable salts thereof), comprising the steps of:
a) contacting a test sample obtained from said subject with a reagent capable of detecting human cancer cells that have MTAP deficiency and/or MTA accumulation; and
b) comparing the test sample with a reference (e.g., a reference sample taken from a non-cancerous or normal control subject),
wherein the presence of MTAP deficiency and/or MTA accumulation in said test sample indicates that the subject will respond to therapeutic treatment with a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof).

In one embodiment, provided is a method of determining if a cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof), comprising the steps of:
a) contacting a test sample obtained from a subject having or having been diagnosed with said cancer with a reagent capable of detecting human cancer cells that have MTAP deficiency and/or MTA accumulation; and
b) comparing the test sample with a reference (e.g., a reference sample taken from a non-cancerous or normal control subject),
   wherein the presence of MTAP deficiency and/or MTA accumulation in said test sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof). In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma. In some embodiments, the method further comprises the step of determining the level of PRMT5 in the cancer cells. The level of expression of PRMT5 can be considered when determining the therapeutically effective dosage of a PRMT5 inhibitor.

In one embodiment, provided is a method of determining the sensitivity of a cancer cell to PRMT5 inhibition (e.g., inhibition with an MTA-uncompetitive PRMT5 inhibitor, e.g., a compound of Table 1, or pharmaceutically acceptable salts thereof), comprising the steps of:
a) assaying the production, level, activity, expression or presence of MTAP), in said cancer cell;
b) comparing the production, level, activity, expression or presence of MTAP in the cancer cell with the production, level, activity, expression or presence of MTAP, respectively, in a non-cancerous or normal control cell,
   wherein a decreased level, activity or expression in the cancer cell indicates MTAP deficiency and wherein MTAP deficiency indicates that said cancer cell is sensitive to the PRMT5 inhibitor.

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment, provided is a method of determining the sensitivity of a cancer cell to a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof), comprising the steps of:
a) assaying for level, activity or expression of the MTAP gene or its gene product in both the cancer cell and a normal control cell, wherein a decreased level, activity or expression in the cancer cell indicates MTAP deficiency; b) assaying for PRMT5 expression in said cancer cell; c) comparing the PRMT5 expression with PRMT5 expression in the cancer cell and a normal control cell; wherein the similarity in PRMT5 expression, and the presence of said MTAP deficiency in said cancer cell, indicates said cell is sensitive to a PRMT5 inhibitor.

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment the provided is a therapeutic method of treating a subject having or having been diagnosed with a cancer (e.g., a cancer associated with MTAP deficiency and/or MTA accumulation) comprising the steps of:
a) assessing the level of MTAP and/or MTA in a test sample obtained from said subject (e.g., by contacting the sample with a reagent capable of detecting human MTAP-deficient and/or MTA-accumulating cancer cells in a test sample obtained from said subject), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor; and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof) to the subject identified in step b).

In one embodiment provided is a therapeutic method of treating a cancer (e.g., a cancer associated with MTAP deficiency and/or MTA accumulation) in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof) to the subject identified in step b).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

In one embodiment provided is a therapeutic method of treating a subject having or having been diagnosed with a cancer associated with MTAP deficiency and/or MTA accumulation comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof) to the subject identified in step b).

In one embodiment provided is a therapeutic method of treating cancer associated with MTAP deficiency and/or MTA accumulation in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive PRMT5 inhibitor e.g., a compound of Table 1, or pharmaceutically acceptable salts thereof) to the subject identified in step b).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

In one embodiment provided is a method of determining if a subject having or having been diagnosed with a cancer associated with MTAP deficiency and/or MTA accumulation will respond to treatment with a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof) comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor (e.g., an MTA-uncompetitive, non-competitive, or mixed mode PRMT5 inhibitor or an MTA-cooperative binding agent).

In one embodiment provided is a method of determining if a cancer associated with MTAP deficiency and/or MTA accumulation will respond to treatment with a PRMT5 inhibitor (e.g., an MTA-uncompetitive PRMT5 inhibitor, e.g., a compound of Table 1, or pharmaceutically acceptable salts thereof) comprising the steps of:
a) assessing the level of MTAP and/or MTA in a test sample obtained from a subject having or having been diagnosed with said cancer (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

### Sample Preparation

Further provided are assays for the detection of MTAP deficiency and/or MTA accumulation. They can include detecting a mutation related to MTAP deficiency and/or MTA accumulation, e.g., in a body fluid such as blood (e.g., serum or plasma) bone marrow, cerebral spinal fluid, peritoneal/pleural fluid, lymph fluid, ascites, serous fluid, sputum, lacrimal fluid, stool, and urine, or in a tissue such as a tumor tissue. The tumor tissue can be fresh tissue or preserved tissue (e.g., formalin fixed tissue, e.g., paraffin-embedded tissue).

Body fluid samples can be obtained from a subject using any of the methods known in the art. Methods for extracting cellular DNA from body fluid samples are well known in the art. Typically, cells are lysed with detergents. After cell lysis, proteins are removed from DNA using various proteases. DNA is then extracted with phenol, precipitated in alcohol, and dissolved in an aqueous solution. Methods for extracting acellular DNA from body fluid samples are also known in the art. Commonly, a cellular DNA in a body fluid sample is separated from cells, precipitated in alcohol, and dissolved in an aqueous solution.

### Detection of PRMT5 Selectivity

Samples, once prepared, can be tested for MTAP deficiency and/or MTA accumulation, either or both of which indicates that the sample is sensitive to treatment with a PRMT5 inhibitor. Cells can be determined to be MTA accumulating by techniques known in the art; methods for detecting MTA include, as a non-limiting example, liquid chromatography-electrospray ionization-tandem mass spectrometry (LC-ESI-MS/MS), as described in Stevens et al. 2010. J. Chromatogr. A. 1217: 3282-3288; and Kirovski et al. 2011 Am. J. Pathol. 178: 1145-1152; and references cited therein. The detection of MTAP deficiency can be done by any number of ways, for example: DNA sequencing, PCR based methods, including RT-PCR, microarray analysis, Southern blotting, Northern blotting, Next Generation Sequencing, and dip stick analysis. In some embodiments, MTAP deficiency is evaluated by any technique known in the art, for example, immunohistochemistry utilizing an anti-MTAP antibody or derivative thereof, and/or genomic sequencing, or nucleic acid hybridization, or amplification utilizing at least one probe or primer comprising a sequence of at least 12 contiguous nucleotides (nt) of the sequence of MTAP wherein the primer is no longer than about 30 nt.

The polymerase chain reaction (PCR) can be used to amplify and identify MTAP deficiency from either genomic DNA or RNA extracted from tumor tissue. PCR is well known in the art and is described in detail in Saiki et al., Science 1988, 239:487.

Methods of detecting MTAP deficiency by hybridization are provided. The method comprises identifying MTAP deficiency in a sample by its inability to hybridize to MTAP nucleic acid. The nucleic acid probe is detectably labeled with a label such as a radioisotope, a fluorescent agent or a chromogenic agent. Radioisotopes can include without limitation; 3H, 32P, 33P and 35S etc. Fluorescent agents can include without limitation: FITC, texas red, rhodamine, etc.

The probe used in detection that is capable of hybridizing to MTAP nucleic acid can be from about 8 nucleotides to about 100 nucleotides, from about 10 nucleotides to about 75 nucleotides, from about 15 nucleotides to about 50 nucleotides, or about 20 to about 30 nucleotides. The kit can also provide instructions for analysis of patient cancer samples, wherein the presence or absence of MTAP deficiency indicates if the subject is sensitive or insensitive to treatment with a PRMT5 inhibitor.

Single stranded conformational polymorphism (SSCP) can also be used to detect MTAP deficiency. This technique is well described in Orita et al., PNAS 1989, 86:2766-2770.

### Measurement of Gene Expression

Evaluation of MTAP deficiency and measurement of MTAP gene expression, and measurement of PRMT5 gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In one embodiment, gene expression is detected and quantitated by hybridization to a probe that specifically hybridizes to the appropriate probe for that biomarker. The probes also can be attached to a solid support for use in high throughput screening assays using methods known in the art.

In one embodiment, the expression level of a gene is determined through exposure of a nucleic acid sample to the probe-modified chip. Extracted nucleic acid is labeled, for example, with a fluorescent tag, preferably during an amplification step.

Hybridization of the labeled sample is performed at an appropriate stringency level. The degree of probe-nucleic acid hybridization is quantitatively measured using a detection device.

Alternatively, any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In one embodiment, the hybridized nucleic acids are detected by detecting one or more labels attached to the sample nucleic acids. The labels can be incorporated by any of a number of means well known to those of skill in the art. However, in one embodiment, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

In one example, the gene expression can be measured through an in-situ hybridization protocol that can detect RNA molecules on a slide containing tissue sections or cells (e.g., through RNAscope®).

Detectable labels suitable for use in the methods disclosed herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 1251, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

### Detection of Polypeptides

Protein levels of MTAP can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

### Adjacent Biomarkers

Near or adjacent to MTAP on chromosome 9 are several other biomarkers. CDKN2A is often, if not usually, deleted along with MTAP. Additional genes or pseudogenes in this region include: C₉orf53, ERVFRD-3, TUBB8P1, KHSRPP1, MIR31, and MIR31HG.

In some embodiments of the methods, the cell that is MTAP-deficient is also deficient in CDKN2A. In some embodiments, the cell that is MTAP-deficient is also deficient in one or more of: CDKN2A, C₉orf53, ERVFRD-3, TUBB8P1, KHSRPP1, MIR31, and MIR31HG.

Thus, in various methods involving a step of evaluating a cell for MTAP deficiency or determining if a cell is MTAP-deficient, this step can comprise the step of determining if the cell is deficient for one or more of these markers: CDKN2A, C₉orf53, ERVFRD-3, TUBB8P1, KHSRPP1, MIR31, and MIR31HG.

Thus, in some embodiments, the disclosure encompasses: A method of determining if a subject having or having been diagnosed with a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from said subject for MTAP deficiency, and evaluating a reference sample from a non-cancerous or normal control subject for MTAP deficiency, wherein MTAP deficiency in the test sample relative to the reference sample indicates that the subject will respond to therapeutic treatment with a PRMT5 inhibitor (e.g., an MTA-uncompetitive PRMT5 inhibitor, e.g., a compound of Table 1, or pharmaceutically acceptable salts thereof); wherein MTAP deficiency is evaluated by evaluating the deficiency of one or more of the following biomarkers: CDKN2A, C₉orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

In some embodiments, the disclosure encompasses: A method of determining if a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from a subject having or having been diagnosed with said cancer for MTAP deficiency, and evaluating a reference sample from a non-cancerous or normal control subject for MTAP deficiency, wherein MTAP deficiency in the test sample relative to the reference sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof); wherein MTAP deficiency is evaluated by evaluating the deficiency of one or more of the following biomarkers: CDKN2A, C₉orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive PRMT5 inhibitor, e.g., a compound of Table 1, or pharmaceutically acceptable salts thereof) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

### Assaying for Biomarkers and PRMT5 Inhibitor Treatment

A number of patient stratification strategies could be employed to find patients likely to be sensitive to PRMT5 inhibition with an MTA-uncompetitive, non-competitive, or mixed mode PRMT5 inhibitor or an MTA-cooperative binding agent (e.g., a PRMT5 inhibitor of the present disclosure, e.g., a compound of Table 1, or pharmaceutically acceptable salts thereof), including but not limited to, testing for MTAP deficiency and/or MTA accumulation.

Once a patient has been assayed for MTAP deficiency and/or MTA accumulation and predicted to be sensitive to treatment with a PRMT5 inhibitor, administration of any PRMT5 inhibitor (e.g., an MTA-uncompetitive, non-competitive, or mixed mode PRMT5 inhibitor or an MTA-cooperative binding agent, e.g., a compound of Table 1, or pharmaceutically acceptable salts thereof) to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

### Kits

In some embodiments provided are kits related to methods of use described herein.

In one embodiment, provided is a kit for predicting the sensitivity of a subject having or having been diagnosed with an MTAP-deficiency-related cancer for treatment with a PRMT5 inhibitor is provided. The kit comprises: i) reagents capable of detecting human MTAP-deficient and/or MTA-accumulating cancer cells; and ii) instructions for how to use said kit.

### EXAMPLES

In order that the invention(s) described herein may be more fully understood, the following examples are set forth. The synthetic and biological examples described in this application are offered to illustrate the compounds, pharmaceutical compositions, and methods provided herein and are not to be construed in any way as limiting their scope. In the synthetic examples below, the descriptions of experimental procedures within a reaction sequence are listed in numerical order.

### Abbreviations

### General

ADDP 1,1′-(azodicarbonyl)dipiperidine
anhy. anhydrous
aq. aqueous
satd. saturated
min(s) minute(s)
hr(s) hour(s)
mL milliliter
mmol millimole(s)
mol mole(s)
MS mass spectrometry
NMR nuclear magnetic resonance
TLC thin layer chromatography
HPLC high-performance liquid chromatography
Me methyl
i-Pr iso-propyl
t-Bu tert-butyl
^{t}BuXPhos 2-di-tert-butylphosphino-2′,4′,6′-triisopropylbiphenyl
Ph phenyl
Et ethyl
Bz benzoyl
RuPhos 2-dicyclohexylphosphino-2′,6′-diisopropoxybiphenyl

### Spectrum

Hz hertz
δ chemical shift
J coupling constant
s singlet
d doublet
t triplet
q quartet
m multiplet
br broad
qd quartet of doublets
dquin doublet of quintets
dd doublet of doublets
dt doublet of triplets

### Solvents and Reagents

DAST Diethylaminosulfurtrifluoride
CHCl₃ chloroform
DCM dichloromethane
DMF dimethylformamide
Et₂O diethyl ether
EtOH ethyl alcohol
EtOAc ethyl acetate
MeOH methyl alcohol
MeCN acetonitrile
PE petroleum ether
THF tetrahydrofuran
DMSO dimethyl sulfoxide
t-BuOK potassium tert-butoxide
9-BBN 9-borabicyclo[3.3.1]nonane
AcOH acetic acid
HCl hydrochloric acid
H₂SO₄ sulfuric acid
NH₄Cl ammonium chloride
KOH potassium hydroxide
NaOH sodium hydroxide
K₂CO₃ potassium carbonate
Na₂CO₃ sodium carbonate
TFA trifluoroacetic acid
Na₂SO₄ sodium sulfate
NaBH₄ sodium borohydride
NaHCO₃ sodium bicarbonate
LiHMDS lithium hexamethyldisilylamide
NaBH₄ sodium borohydride
Et₃N triethylamine
Py pyridine
PCC pyridinium chlorochromate
DMAP 4-(dimethylamino)pyridine
DIPEA N,N-diisopropylethylamine
BINAP 2,2′-bis(diphenylphosphanyl)-1,1′-binaphthyl
dppf 1,1′-bis(diphenylphosphino)ferrocene
PEP Phospho(enol)pyruvic acid
LDH Lactate Dehydrogenase
DTT DL-Dithiothreitol
BSA Bovine Serum Albumin
NADH β-Nicotinamide adenine dinucleotide, reduced
Pd(t-Bu₃P)₂ bis(tri-tert-butylphosphine)palladium(0)
AcCl acetyl chloride
i-PrMgCl Isopropylmagnesium chloride
TBSCl tert-Butyl(chloro)dimethylsilane
(i-PrO)₄Ti titanium tetraisopropoxide
BHT 2,6-di-t-butyl-4-methylphenoxide
BzCl benzoyl chloride
CsF cesium fluoride
DCC dicyclohexylcarbodiimide
DMP Dess-Martin periodinane
EtMgBr ethylmagnesium bromide
EtOAc ethyl acetate
TEA triethylamine
AlaOH alanine
TBAF tetra-n-butylammonium fluoride
TBS t-butyldimethylsilyl
TMS trimethylsilyl
TMSCF₃ (Trifluoromethyl)trimethylsilane
Ts p-toluenesulfonyl
Bu butyl
Ti(OⁱPr)₄ tetraisopropoxytitanium
LAH Lithium Aluminium Hydride
LDA lithium diisopropylamide
LiOH·H₂O lithium hydroxide hydrates
MAD methyl aluminum bis(2,6-di-t-butyl-4-methylphenoxide)
NBS N-bromosuccinimide
Na₂SO₄ sodium sulfate
Na₂S₂O₃ sodium thiosulfate
PE petroleum ether
MeCN acetonitrile
Boc t-butoxycarbonyl
MTBE methyl tert-butyl ether
DIAD diisopropyl azodicarboxylate

### General Experimental Notes

In the following examples, the chemical reagents were purchased from commercial sources (such as Alfa, Acros, Sigma Aldrich, TCI and Shanghai Chemical Reagent Company), and used without further purification.

In some examples, purification of intermediates and final compounds was performed using HPLC (H₂O-MeOH; Agilent 1260 Infinity systems equipped with DAD and mass-detectors. Waters Sunfire C18 OBD Prep Column, 100A, 5 μm, 19 mm×100 mm with SunFire C18 Prep Guard Cartridge, 100A, 10 μm, 19 mm×10 mm) The material was dissolved in 0.7 mL DMSO. Flow: 30 mL/min. Purity of the obtained fractions was checked via the analytical LCMS. Spectra were recorded for each fraction as it was obtained straight after chromatography in the solution form. The solvent was evaporated under the N₂ flow upon heating to 80° C. On the basis of post-chromatography LCMS analysis fractions were united. Solid fractions were dissolved in 0.5 mL MeOH and transferred into pre-weighted marked vials. Obtained solutions were again evaporated under the N₂ flow upon heating to 80° C. After drying, products were subjected to lyophilization using acetonitrile-water mixtures and finally characterized by LCMS and ¹H NMR.

Nuclear magnetic resonance (NMR) spectra were recorded using Brucker AVANCE DRX 500, Bruker 400 spectrometer or Varian UNITYplus 400. Chemical shifts for protons were reported as parts per million in 8 scale using solvent residual peak (CHCl₃: 7.27 ppm) (methanol-d₄: 3.31 ppm) (DMSO-d₆: 2.50 ppm) or tetramethylsilane (0.00 ppm) as internal standards. Chemical shifts of ¹³C NMR spectra were reported in ppm from the central peak of CDCl₃ (77.00 ppm) (methanol-d₄: 49.15 ppm) (DMSO-d₆: 39.51 ppm) on the 8 scale.

Data are represented as follows: chemical shift, multiplicity (s=singlet, d=doublet, t=triplet, q=quartet, qn=quintuplet, sx=sextet, sp=septuplet, m=multiplet, br=broad), coupling constant (J, Hz) and integration.

In certain examples, mass spectra were recorded on an Agilent 1100 Series LC/MSD system with DAD\ELSD and Agilent LC\MSD VL (G1956A), SL (G1956B) mass-spectrometer or an Agilent 1200 Series LC/MSD system with DAD\ELSD and Agilent LC\MSD SL (G6130A), SL (G6140A) mass-spectrometer.

All the LC/MS data were obtained using positive/negative mode switching.
Column Zorbax SB-C18 1.8 μm 4.6×15 mm Rapid Resolution cartridge (PN 821975-932)
Mobile phase A—acetonitrile, 0.1% formic acid
   B—water (0.1% formic acid)
Flow rate 3 ml/min
Gradient 0 min—100% B
0.01 min—100% B
1.5 min—0% B
1.8 min—0% B
1.81 min—100% B
Injection volume 1 μl
Ionization mode atmospheric pressure chemical ionization (APCI)
Scan range m/z 80-1000.

### Other Exemplary Analytical LC/MS Instruments and Conditions are Described Below:

Instrument: Agilent LC1100-MS6100 series G1956B; Column: Xbridge Shield RP-18, 50*2.1 mm*5 μm; Mobile Phase A: H₂O with 0.05% NH₃—H₂O (v %); Mobile Phase B: MeCN; Flow rate: 1.0 mL/min; Wavelength: UV 220 nm, 254 nm; Column temperature: 30° C.; MS ionization: ESI.
0-30CD: Gradient: B from 0%˜30% over 2 minutes and holding at 30% for 0.48 minutes;
0-60CD: Gradient: B from 0%˜60% over 2 minutes and holding at 60% for 0.48 minutes;
10-80CD: Gradient: B from 10%˜80% over 2 minutes and holding at 80% for 0.48 minutes;
30-90CD: Gradient: B from 30%˜90% over 2 minutes and holding at 90% for 0.48 minutes;
50-100CD: Gradient: B from 50%1⁰⁰⁰% over 2 minutes and holding at 100% for 0.48 minutes.

Instrument: Agilent LC1100-MS6100 series G1956B; Column: Xtimate C18, 30*2.1 mm*3 m; Mobile Phase A: H₂O with 0.0375% TFA (v %); Mobile Phase B: MeCN with 0.01875% TFA (v %): Flow rate: 0.8 mL/min; Wavelength: UV 220 nm, 254 nm; Column temperature: 50° C.; MS ionization: ESI.
0-30AB: Gradient: B from 0%˜30% over 3 minutes and holding at 30% for 0.5 minutes;
0-60AB: Gradient: B from 0%˜60% over 3 minutes and holding at 30% for 0.5 minutes;
10-80AB: Gradient: B from 10%˜80% over 3 minutes and holding at 30% for 0.5 minutes;
30-90AB: Gradient: B from 0%˜30% over 3 minutes and holding at 30% for 0.5 minutes;
50-100AB: Gradient: B from 50%1⁰⁰⁰% over 3 minutes and holding at 100% for 0.5 minutes.

Instrument: Shimadzu LC20-MS2010; Column: Agilent Pursit 5 C18 20*2.0 mm; Mobile Phase A: H₂O with 0.0375% of TFA (v %); Mobile Phase B: MeCN with 0.01875% of TFA (v %); Gradient: B from 5˜95% over 0.7 minutes and holding at 95% for 0.4 minutes; Flow Rate: 1.5 mL/min; Wavelength: UV 220 nm, 254 nm, 215 nm; Column temperature: 50° C.; MS ionization: ESI.

Instrument: Shimadzu LC20-MS2020; Column: Agilent Pursit 5 C18 20*2.0 mm; Mobile Phase A: H₂O with 0.0375% of TFA (v %); Mobile Phase B: MeCN with 0.01875% of TFA (v %); Gradient: B from 5˜95% over 0.7 minutes and holding at 95% for 0.4 minutes; Flow Rate: 1.5 mL/min; Wavelength: UV 220 nm, 254 nm; Column temperature: 50° C.; MS ionization: ESI.

### Exemplary HPLC Instruments and Conditions

Instrument: Shimadzu LC20; Column: YMC-Pack ODS-A 150*4.6 mm; Mobile Phase A: H₂O with 0.06875% TFA (v %); Mobile Phase B: MeCN with 0.0625% TFA (v %); Flow rate: 1.5 mL/min; Wavelength: UV 220 nm, 215 nm, 254 nm; Column temperature: 40° C.
0-30: Gradient: B from 0˜30% over 10 minutes and holding at 30% for 5 minutes;
0˜60: Gradient: B from 0˜60% over 10 minutes and holding at 60% for 5 minutes;
0-95: Gradient: B from 0˜95% over 10 minutes and holding at 95% for 5 minutes;
10-80: Gradient: B from 10˜80% over 10 minutes and holding at 80% for 5 minutes;
30-90: Gradient: B from 30˜90% over 10 minutes and holding at 90% for 5 minutes;
50-100: Gradient: B from 50˜100% over 10 minutes and holding at 100% for 5 minutes.

Instrument: Shimadzu LC20; Column: Xbridge Shield RP-18 50*2.1 mm, 5 μm; Mobile Phase A: H₂O with 0.01% NH₃—H₂O; Mobile Phase B: MeCN; Flow Rate: 1.2 mL/min; Wavelength: UV 220 nm, 215 nm, 254 nm; Column temperature: 40° C.
0-30CD: Gradient: B from 0˜30% over 6 minutes and holding at 30% for 2 minutes;
0-60CD: Gradient: B from 0˜60% over 6 minutes and holding at 60% for 2 minutes;
10-80CD: Gradient: B from 10˜80% over 6 minutes and holding at 80% for 2 minutes;
30-90CD: Gradient: B from 30˜90% over 6 minutes and holding at 90% for 2 minutes;
50-100CD: Gradient: B from 10˜80% over 6 minutes and holding at 100% for 2 minutes.

Instrument: Shimadzu LC20; Column: Ultimate C18 50*3 mm, 3 μm; Mobile Phase A: H₂O with 0.06875% TFA (v %); Mobile Phase B: MeCN with 0.0625% TFA (v %); Flow Rate: 1.2 mL/min; Wavelength: UV 220 nm, 215 nm, 254 nm; Column temperature: 40° C.
0-30AB: Gradient: B from 0˜30% over 2.5 minutes and holding at 30% for 0.75 minutes;
0-60AB: Gradient: B from 0˜60% over 2.5 minutes and holding at 60% for 0.75 minutes;
5-95AB: Gradient: B from 5˜95% over 2.5 minutes and holding at 95% for 0.75 minutes.

Instrument: Shimadzu LC20; Column: Ultimate C18 50*3 mm, 3 μm; Mobile Phase A: H₂O with 0.06875% TFA (v %); Mobile Phase B: MeCN with 0.0625% TFA (v %); Flow Rate: 1.2 mL/min; Wavelength: UV 220 nm, 215 nm, 254 nm; Column temperature: 40° C.
10-80AB: Gradient: B from 10˜80% over 4 minutes and holding at 80% for 2 minutes.

### Exemplary TLC, Concentration and Normal Phase Chromatography.

Analytical thin layer chromatography (TLC) was performed with silica gel 60 F254 aluminum plates. Visualization was done under a UV lamp (254 nm) and by iodine or immersion in ethanolic phosphomolybdic acid (PMA) or potassium permanganate (KMnO₄), followed by heating using a heat gun. Organic solutions were concentrated by rotary evaporation at 20˜40° C. Purification of reaction products were generally done by flash column chromatography with 230-400 mesh silica gel or Agela flash silica column.

### Exemplary Chiral SFC Analytical Methods

Column: Chiralpak AD-3 150×4.6 mm I.D., 3 m; Mobile phase: A: supercritical CO₂; Mobile phase B: EtOH (0.05% DEA); Gradient: from 5% to 40% of B in 5 min and hold 40% for 2.5 min, then 5% of B for 2.5 min; Flow rate: 2.5 mL/min; Column temperature: 35° C.; ABPR: 1500 psi.

Column: Chiralpak AD-3 100×4.6 mm I.D., 3 m; Mobile phase: A: supercritical CO₂ Mobile phase B: EtOH (0.1% ethanolamine); Gradient: from 5% to 40% of B in 4.5 min and hold 40% for 2.5 min, then 5% of B for 1 min; Flow rate: 2.8 mL/min; Column temperature: 40° C.

### Exemplary Preparative HPLC Separation Methods

Basic condition (NH₃—H₂O): Instrument: Gilson GX-281 Liquid Handler, Gilson 322 Pump, Gilson 156 UV Detector; Column: Waters Xbridge 150×25 mm×5 m; Mobile phase A: H₂O with 0.05% NH₃—H₂O (v %); Mobile phase B: MeCN; Gradient: B from 22% to 52% in 9.5 min, hold 100% B for 1 min; Flow Rate: 25 mL/min; Column Temperature: 30° C.; Wavelength: 220 nm, 254 nm.

Acid condition (HCOOH): Instrument: Gilson GX-281 Liquid Handler, Gilson 322 Pump, Gilson 156 UV Detector; Column: Agela Durashell C18 150*25 mm 5 μm; Mobile phase A: H₂O (0.0225% HCOOH); Mobile phase B: MeCN; Gradient: B from 7% to 37% in 9 min, hold 100% B for 0 min; Flow Rate: 25 mL/min; Column Temperature: 30° C.; Wavelength: 220 nm, 254 nm.

Acid condition (HCl): Gilson GX-281 Liquid Handler, Gilson 322 Pump, Gilson 156 UV Detector; Column: Xtimate C18 150*25 mm*5 m; Mobile phase A: H₂O with 0.05% HCl (v %); Mobile phase B: MeCN; Gradient: B from 0% to 30% in 6.5 min, hold 100% B for 2.5 min; Flow Rate: 25 mL/min; Column Temperature: 30° C.; Wavelength: 220 nm, 254 nm).

Neutral condition (NH₄HCO₃): (Instrument: Gilson GX-281 Liquid Handler, Gilson 322 Pump, Gilson 156 UV Detector; Column: Waters Xbridge 150×25 mm×5 μm; Mobile phase A: H₂O with 10 mmol NH₄HCO₃; Mobile phase B: MeCN; Gradient: B from 39% to 69% in 10 min, hold 100% B for 2.5 min; Flow Rate: 25 mL/min; Column Temperature: 30° C.; Wavelength: 220 nm, 254 nm).

### Exemplary Large-Scale Separation

Basic condition: Instrument: Shimadzu LC-8A Pumps, Shimadzu SCL-10A VP System Controller, Shimadzu SPD-20AV UV/VIS Detector; Column: Phenomenex Gemini C18 250*50 mm*10 m; Mobile phase A: water (0.04% NH₃—H₂O+10 mM NH₄HCO₃); Mobile phase B: MeCN; Gradient: B from 65% to 95% in 26 min, hold 100% B for 3 min; Flow Rate: 110 mL/min; Column Temperature: 30° C.; Wavelength: 220 nm, 254 nm.

Acid condition (TFA): Instrument: Shimadzu LC-20AP Pumps, Shimadzu CBM-20A System Controller Shimadzu SPD-20AV UV/VIS Detector; Column: Phenomenex luna C18 250×50 mm×10 m; Mobile phase A: H₂O with 0.1% TFA (v %); Mobile phase B: MeCN; Gradient: B from 0% to 25% in 15 min, hold 100% B for 4 min; Flow Rate: 120 mL/min; Column Temperature: 30° C.; Wavelength: 220 nm, 254 nm.

### Exemplary Preparative Chiral SFC Method:

Exemplary chiral columns available for use in the separation/purification of the enantiomers/diastereomers provided herein include, but are not limited to, CHIRALPAK® AD-10, CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

In certain examples, the chiral separation was performed under the following conditions: Instrument: Thar 80; Column: Daicel Chiralpak AD. 250×30 mm I.D. 10 m; Mobile phase: supercritical C₀₂/MeOH (0.1% NH₃—H₂O, v %)=60/40; Flow Rate: 70 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative pyrazoles that have been listed herein. The compounds provided herein may be prepared from known or commercially available starting materials and reagents by one skilled in the art of organic synthesis.

Exemplary general method for preparative HPLC: Column: Waters RBridge prep 10 m C18, 19*250 mm. Mobile phase: acetonitrile, water (NH₄HCO₃) (30 L water, 24 g NH₄HCO₃, 30 mL NH₃·H₂O). Flow rate: 25 mL/min

Exemplary general method for analytical HPLC: Mobile phase: A: water (10 mM NH₄HCO₃), B: acetonitrile Gradient: 5%-95% B in 1.6 or 2 min Flow rate: 1.8 or 2 mL/min; Column: XBridge C18, 4.6*50 mm, 3.5 m at 45° C.

### Miscellaneous Piperidine Intermediates Syntheses

### 3EEE. 5-((2R,5S)-5-methylpiperidin-2-yl)-2-(1-(pyrrolidin-1-yl)propan-2-yl)benzo[d]thiazole

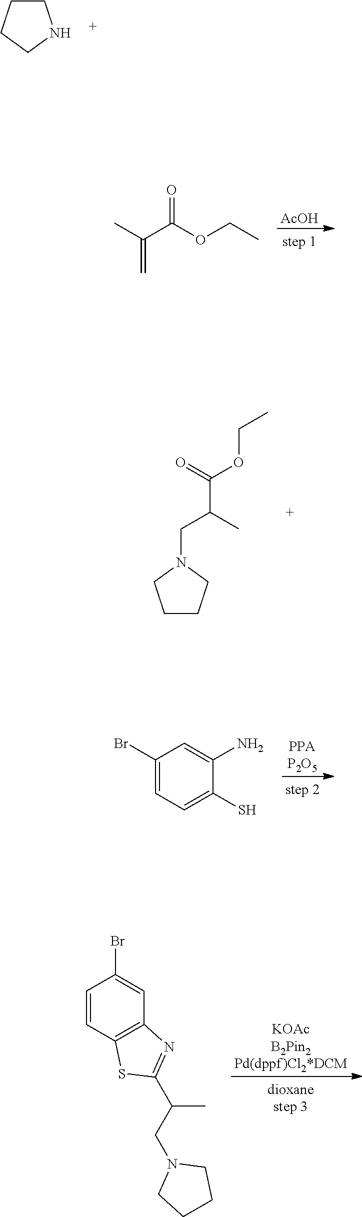

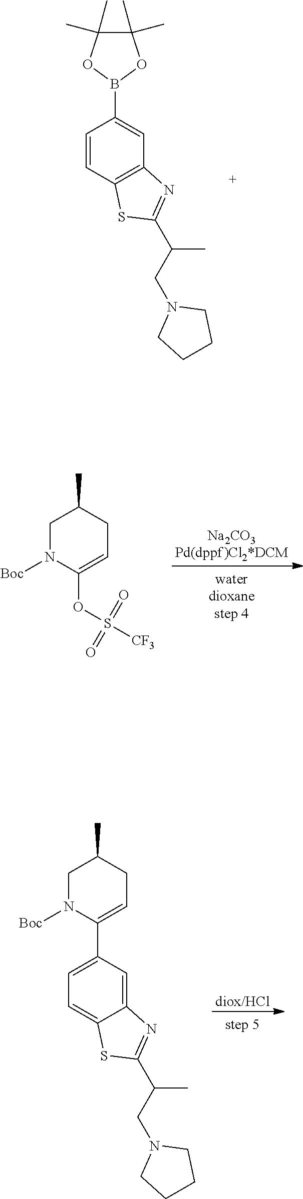 [SMILES: CC(CN1CCCC1)c1nc2cc(B3OC(C)(C)C(C)(C)O3)ccc2s1 ; C[C@H]1CC=C(OS(=O)(=O)C(F)(F)F)N(C(=O)OC(C)(C)C)C1 ; "Na2CO3Pd(dppf)Cl2*DCM" ; CC(CN1CCCC1)c1nc2cc(C3=CC[C@H](C)CN3C(=O)OC(C)(C)C)ccc2s1 ; "diox/HCl"]

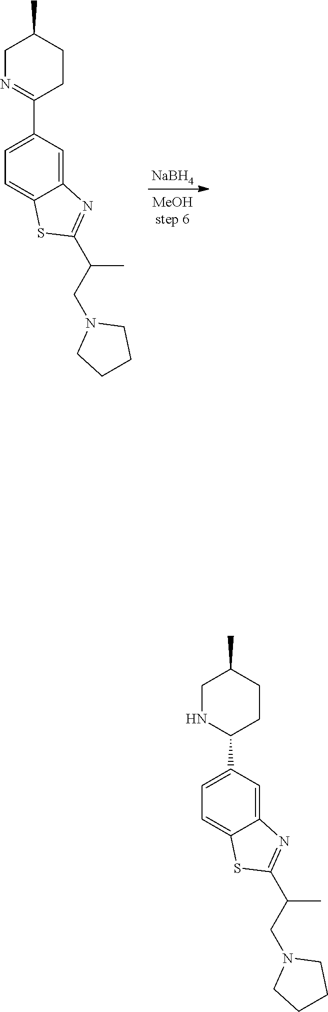 [SMILES: CC(CN1CCCC1)c1nc2cc(C3=NC[C@@H](C)CC3)ccc2s1 ; "NaBH4" ; CC(CN1CCCC1)c1nc2cc([C@H]3CC[C@H](C)CN3)ccc2s1]

### Step 1: Synthesis of ethyl 2-methyl-3-(pyrrolidin-1-yl)propanoate

Pyrrolidine (7 g, 98.42 mmol, 8.18 mL) and ethyl 2-methylprop-2-enoate (10.21 g, 89.48 mmol, 11.14 mL) were mixed together and acetic acid (537.32 mg, 8.95 mmol, 512.22 L) was added thereto. The resulting reaction mixture was stirred at 70° C. for 17 hr. After completion the reaction mixture was diluted by hexane (30 mL) and washed with NaHCO₃. This solution was filtered through the pad of silica that was washed with hexane (3*10 mL). The combined organic solution was evaporated under reduced pressure to afford ethyl 2-methyl-3-pyrrolidin-1-yl-propanoate (13 g, 70.17 mmol, 78.42% yield).

¹H NMR (400 MHz, CDCl₃) δ (ppm) (d, 3H), 1.19 (t, 3H), 1.67 (m, 4H), 2.43 (m, 5H), 2.58 (m, 1H), 2.69 (m, 1H), 4.09 (m, 2H).

### Step 2: Synthesis of 5-bromo-2-(1-(pyrrolidin-1-yl)propan-2-yl)benzo[d]thiazole

Prepared by general procedure scheme 4.1 step 1A. Yield: 5 g (28.48%).

LCMS(ESI): [M]⁺ m/z: calcd 325.2; found 326.2; Rt=0.760 min.

### Step 3: Synthesis of 2-(1-(pyrrolidin-1-yl)propan-2-yl)-5-(4,4,5,5-tetramethyl-1,3,2-dioxaborolan-2-yl)benzo[d]thiazole

Prepared by general procedure scheme 4.1 step 2. Yield: 7.7 g of crude.

LCMS(ESI): [M]⁺ m/z: calcd 372.2; found 373.2; Rt=1.154 min.

### Step 4: Synthesis of (3S)-tert-butyl 3-methyl-6-(2-(1-(pyrrolidin-1-yl)propan-2-yl)benzo[d]thiazol-5-yl)-3,4-dihydropyridine-1(2H)-carboxylate

Prepared by general procedure scheme 4.1 step 3. Yield: 9 g of crude.

LCMS(ESI): [M]⁺ m/z: calcd 441.2; found 442.2; Rt=1.273 min.

### Step 5: Synthesis of 5-((S)-5-methyl-3,4,5,6-tetrahydropyridin-2-yl)-2-(1-(pyrrolidin-1-yl)propan-2-yl)benzo[d]thiazole

tert-Butyl (3S)-3-methyl-6-[2-[1-methyl-2-pyrrolidin-1-yl-ethyl]-1,3-benzothiazol-5-yl]-3,4-dihydro-2H-pyridine-1-carboxylate (4.98 g, 11.27 mmol) was dissolved in the diox/HCl (30 mL) and the resulting reaction mixture was stirred at 25° C. for 16 hr. After the completion the solvent was evaporated under reduced pressure to afford crude 2-[1-methyl-2-pyrrolidin-1-yl-ethyl]-5-[(3S)-3-methyl-1,2,3,4-tetrahydropyridin-6-yl]-1,3-benzothiazole that was directly used in the next step.

LCMS(ESI): [M]⁺ m/z: calcd 341.2; found 342.2; Rt=0.678 min.

### Step 6: Synthesis of 5-((2R,5S)-5-methylpiperidin-2-yl)-2-(1-(pyrrolidin-1-yl)propan-2-yl)benzo[d]thiazole

Prepared by general procedure scheme 4.1 step 5. Yield: 3 g of crude.

LCMS(ESI): [M]⁺ m/z: calcd 343.2; found 344.2; Rt=0.586 min.

### 3FFF. 5-((2R,5S)-5-methylpiperidin-2-yl)-2-(1,5,5-trimethylpyrrolidin-3-yl)benzo[d]thiazole

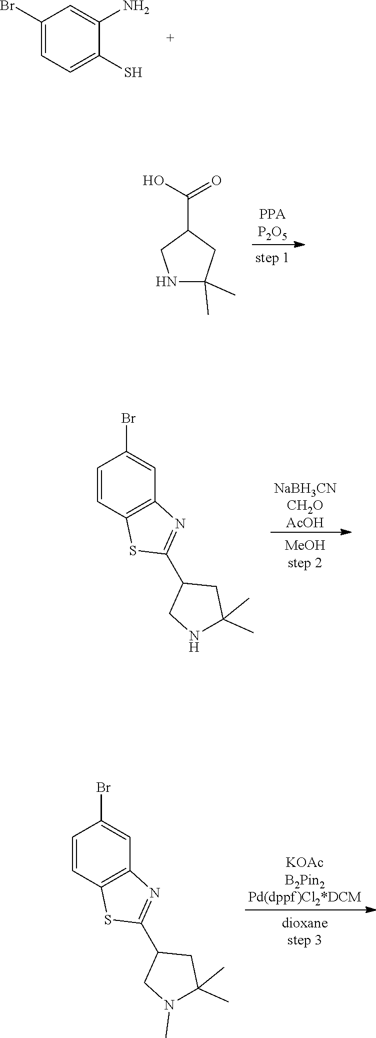

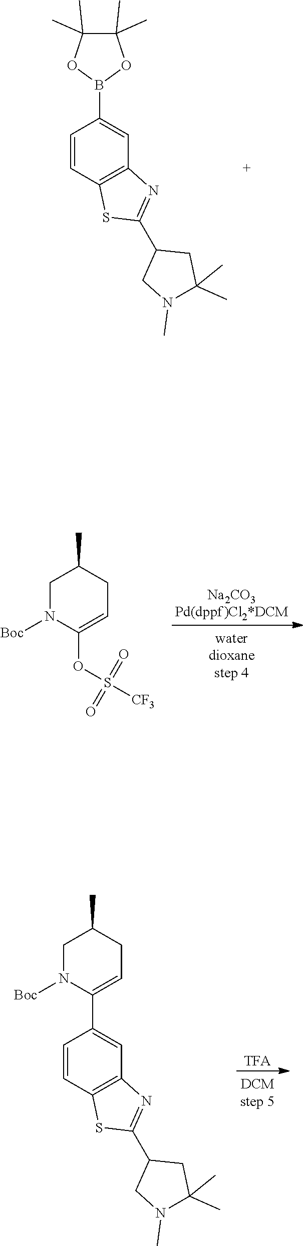

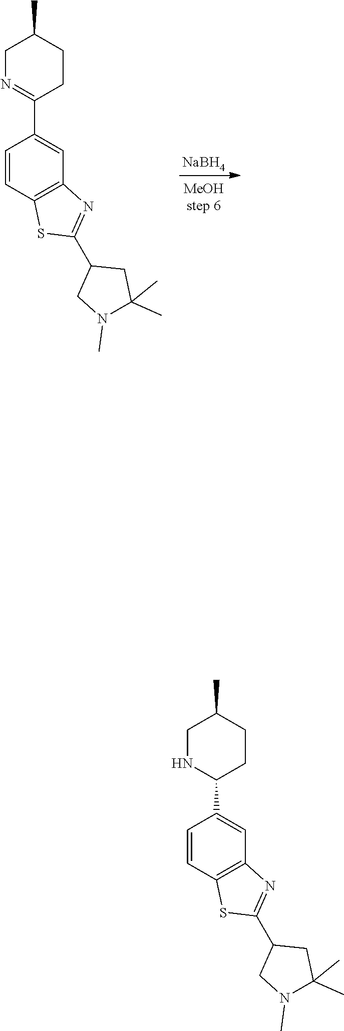 [SMILES: C[C@H]1CCC(c2ccc3sc(C4CN(C)C(C)(C)C4)nc3c2)=NC1 ; "NaBH4" ; C[C@H]1CC[C@H](c2ccc3sc(C4CN(C)C(C)(C)C4)nc3c2)NC1]

### Step 1: Synthesis of 5-bromo-2-(5,5-dimethylpyrrolidin-3-yl)benzo[d]thiazole

Prepared by general procedure scheme 4.1 step 1A. Yield: 9.5 g (98.88%).

LCMS(ESI): [M]⁺ m/z: calcd 311.2; found 312.2; Rt=0.949 min.

### Step 2: Synthesis of 5-bromo-2-(1,5,5-trimethylpyrrolidin-3-yl)benzo[d]thiazole

Formaldehyde, 37% w/w aq. soln., stab. with 7-8% MeOH (3.47 g, 42.73 mmol, 3.20 mL, 37% purity) and acetic acid (3.67 g, 61.05 mmol, 3.49 mL) were added to a stirred solution of 5-bromo-2-(5,5-dimethylpyrrolidin-3-yl)-1,3-benzothiazole (9.5 g, 30.52 mmol) in MeOH (250 mL) at 25° C. The resulting mixture was stirred at 25° C. for 1 hr, then sodium cyan borohydride (1.92 g, 30.52 mmol) was added in one portion at 25° C. (foaming!). The reaction mixture was stirred at 25° C. for 18 hr, and then concentrated in vacuum. The residue was diluted with 10% aqueous sodium hydroxide solution (100 ml) and extracted with DCM (2*100 ml). The combined organic extracts were dried over sodium sulphate and concentrated in vacuum to afford crude 5-bromo-2-(1,5,5-trimethylpyrrolidin-3-yl)-1,3-benzothiazole (9.5 g, 29.21 mmol, 95.69% yield) as light-yellow solid, which was used directly in the next step.

LCMS(ESI): [M]⁺ m/z: calcd 325.2; found 326.2; Rt=2.518 min.

### Step 3: Synthesis of 5-(4,4,5,5-tetramethyl-1,3,2-dioxaborolan-2-yl)-2-(1,5,5-trimethylpyrrolidin-3-yl)benzo[d]thiazole

Prepared by general procedure scheme 4.1 step 2. Yield: 5.7 g of crude.

LCMS(ESI): [M]⁺ m/z: calcd 372.2; found 373.2; Rt=1.171 min.

### Step 4: Synthesis of (3S)-tert-butyl 3-methyl-6-(2-(1,5,5-trimethylpyrrolidin-3-yl)benzo[d]thiazol-5-yl)-3,4-dihydropyridine-1(2H)-carboxylate

Prepared by general procedure scheme 4.1 step 3. Yield: 16.5 g of crude.

LCMS(ESI): [M]⁺ m/z: calcd 441.2; found 442.2; Rt=1.289 min.

### Step 5: Synthesis of 5-((S)-5-methyl-3,4,5,6-tetrahydropyridin-2-yl)-2-(1,5,5-trimethylpyrrolidin-3-yl)benzo[d]thiazole

Prepared by general procedure scheme 4.1 step 4. Yield: 4.7 g of crude.

LCMS(ESI): [M]⁺ m/z: calcd 341.2; found 342.2; Rt=0.635 min.

### Step 6: Synthesis of 5-((2R,5S)-5-methylpiperidin-2-yl)-2-(1,5,5-trimethylpyrrolidin-3-yl)benzo[d]thiazole

Sodium borohydride (780.94 mg, 20.64 mmol, 727.13 μL) was added in one portion at 0° C. to a stirred solution of 5-[(3S)-3-methyl-2,3,4,5-tetrahydropyridin-6-yl]-2-(1,5,5-trimethylpyrrolidin-3-yl)-1,3-benzothiazole (4.7 g, 13.76 mmol) in MeOH (60 mL). The reaction mixture was stirred at 0° C. for 1 hr, and then concentrated in vacuum. The residue was diluted with water (25 ml) and extracted with DCM (2*50 ml). The combined organic extracts were dried over sodium sulphate and concentrated in vacuum to afford crude 5-[(2R,5S)-5-methyl-2-piperidyl]-2-(1,5,5-trimethylpyrrolidin-3-yl)-1,3-benzothiazole (4 g, 11.64 mmol, 84.61% yield) as light-brown gum, which was used directly in the next step. Additionally, 1 g of crude amine was purified by reverse phase HPLC (column: XBridge C18 100×19 mm, 5 um; mobile phase: 35-60% 0-5 min H₂O/MeCN/0.1% NH₄OH, flow rate: 30 ml/min (loading pump 4 ml/min MeCN)) to afford pure 5-[(2R,5S)-5-methyl-2-piperidyl]-2-(1,5,5-trimethylpyrrolidin-3-yl)-1,3-benzothiazole (530 mg, 1.54 mmol, 11.21% yield) as red gum.

LCMS(ESI): [M]⁺ m/z: calcd 343.2; found 344.2; Rt=0.789 min.

### 3GGG. (S)—N,N-dimethyl-1-(5-((2R,5S)-5-methylpiperidin-2-yl)benzo[d]thiazol-2-yl)propan-2-amine

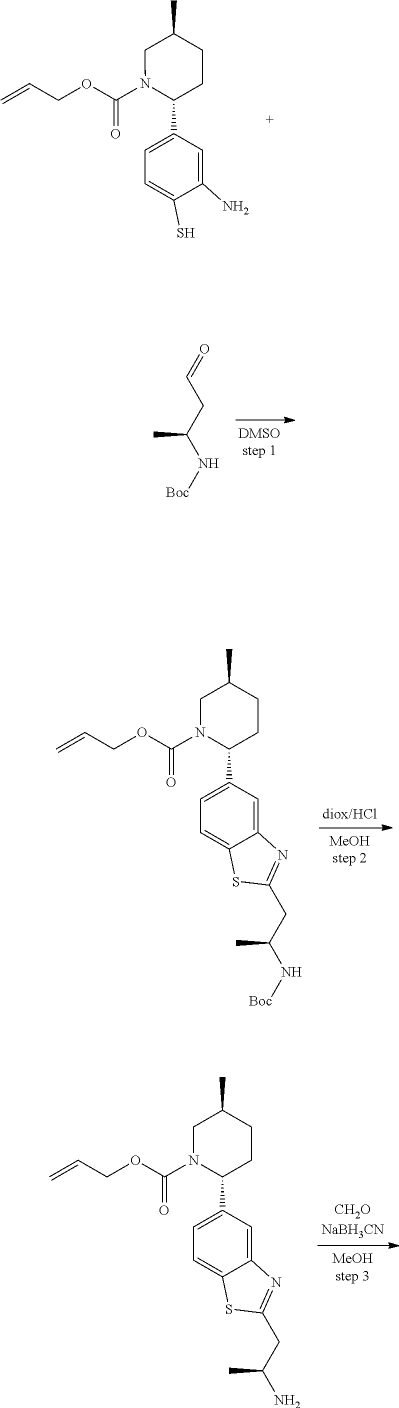 [SMILES: C=CCOC(=O)N1C[C@@H](C)CC[C@@H]1c1ccc(S)c(N)c1 ; C[C@@H](CC=O)NC(=O)OC(C)(C)C ; C=CCOC(=O)N1C[C@@H](C)CC[C@@H]1c1ccc2sc(C[C@H](C)NC(=O)OC(C)(C)C)nc2c1 ; C=CCOC(=O)N1C[C@@H](C)CC[C@@H]1c1ccc2sc(C[C@H](C)N)nc2c1]

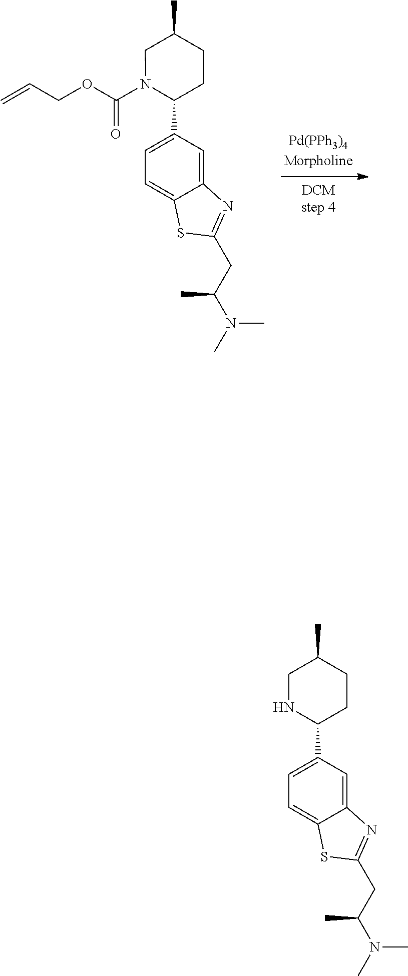 [SMILES: C=CCOC(=O)N1C[C@@H](C)CC[C@@H]1c1ccc2sc(C[C@H](C)N(C)C)nc2c1 ; C[C@H]1CC[C@H](c2ccc3sc(C[C@H](C)N(C)C)nc3c2)NC1]

### Step 1: Synthesis of (2R,5S)-allyl 2-(2-((S)-2-((tert-butoxycarbonyl)amino)propyl)benzo[d]thiazol-5-yl)-5-methylpiperidine-1-carboxylate

Allyl (2R,5S)-2-(3-amino-4-sulfanyl-phenyl)-5-methyl-piperidine-1-carboxylate (2.3 g, 7.51 mmol) and tert-butyl N-[(1S)-1-methyl-3-oxo-propyl]carbamate (1.6 g, 8.55 mmol) were mixed together in DMSO (20 mL) under argon atmosphere and the resulting solution was heated at 90° C. for 16 hr. Upon completion, the reaction mixture was quenched with ethyl acetate (30 mL). The organic phase was extracted with brine (3*30 mL). The organic phase was dried over Na₂SO₄ and concentrated under reduced pressure. The desired product allyl (2R,5S)-5-methyl-2-[2-[(2S)-2-(tert-butoxycarbonylamino)propyl]-1,3-benzothiazol-5-yl]piperidine-1-carboxylate (3.5 g, 7.39 mmol, 98.45% yield) was isolated.

LCMS(ESI): [M]⁺ m/z: calcd 473.2; found 474.2; Rt=1.540 min.

### Step 2: Synthesis of (2R,5S)-allyl 2-(2-((S)-2-aminopropyl)benzo[d]thiazol-5-yl)-5-methylpiperidine-1-carboxylate

Allyl (2R,5S)-5-methyl-2-[2-[(2S)-2-(tert-butoxycarbonylamino)propyl]-1,3-benzothiazol-5-yl]piperidine-1-carboxylate (3.50 g, 7.39 mmol) was stirred in MeOH (30 mL) and diox/HCl (15 mL) for 16 hr at 25° C. Upon completion, the reaction mixture was evaporated, the crude product was quenched with water (20 mL) and neutralized by NaHCO₃ to pH=8. The aqueous phase was extracted with CHCl₃ (2*20 mL). The combined organic phase was dried over Na₂SO₄ and concentrated under reduced pressure. The desired product allyl (2R,5S)-5-methyl-2-[2-[(2S)-2-aminopropyl]-1,3-benzothiazol-5-yl]piperidine-1-carboxylate (2.5 g, 6.69 mmol, 90.57% yield) was isolated.

LCMS(ESI): [M]⁺ m/z: calcd 373.2; found 374.2; Rt=1.146 min.

### Step 3: Synthesis of (2R,5S)-allyl 2-(2-((S)-2-(dimethylamino)propyl)benzo[d]thiazol-5-yl)-5-methylpiperidine-1-carboxylate

Allyl (2R,5S)-5-methyl-2-[2-[(25)-2-aminopropyl]-1,3-benzothiazol-5-yl]piperidine-1-carboxylate (2.5 g, 6.69 mmol) and formaldehyde, 37% w/w aq. soln., stab. with 7-8% MeOH (5.42 g, 180.32 mmol, 5 mL) were mixed together in MeOH (50 mL) and the resulting solution was cooled to 5° C. in an ice bath. Sodium cyan borohydride (2.10 g, 33.47 mmol) was added to the previous solution and the resulting mixture was allowed to warm to rt and stirred overnight. Upon completion, the reaction mixture was evaporated; the crude product was quenched with water (30 mL). The aqueous phase was extracted with CHCl₃ (2*30 mL). The combined organic phase was dried over Na₂SO₄ and concentrated under reduced pressure. The desired product allyl (2R,5S)-5-methyl-2-[2-[(25)-2-(dimethylamino)propyl]-1,3-benzothiazol-5-yl]piperidine-1-carboxylate (2.5 g, 6.23 mmol, 93.01% yield) was isolated.

LCMS(ESI): [M]⁺ m/z: calcd 401.2; found 402.2; Rt=0.991 min.

### Step 4: Synthesis of (S)—N,N-dimethyl-1-(5-((2R,5S)-5-methylpiperidin-2-yl)benzo[d]thiazol-2-yl)propan-2-amine

To a stirred solution of allyl (2R,5S)-5-methyl-2-[2-[(25)-2-(dimethylamino)propyl]-1,3-benzothiazol-5-yl]piperidine-1-carboxylate (2.5 g, 6.23 mmol) and morpholine (1.10 g, 12.58 mmol, 1.1 mL) in DCM (25 mL) was added palladium (0) tetrakis(triphenylphosphine) (0.3 g, 259.61 μmol). The resulting suspension was degassed with argon at 25° C. for 0.5 hr. The reaction mixture was stirred at 25° C. for 6 hr. Upon completion, the reaction mixture was extracted with water (2*25 mL). The organic phase was dried over Na₂SO₄ and concentrated under reduced pressure. The desired product (2S)—N,N-dimethyl-1-[5-[(2R,5S)-5-methyl-2-piperidyl]-1,3-benzothiazol-2-yl]propan-2-amine (1.9 g, 5.98 mmol, 96.13% yield) was isolated.

LCMS(ESI): [M]⁺ m/z: calcd 317.2; found 318.2; Rt=0.501 min.

### 3HHH. N,N,2-trimethyl-1-(5-((2R,5S)-5-methylpiperidin-2-yl)benzo[d]thiazol-2-yl)propan-2-amine

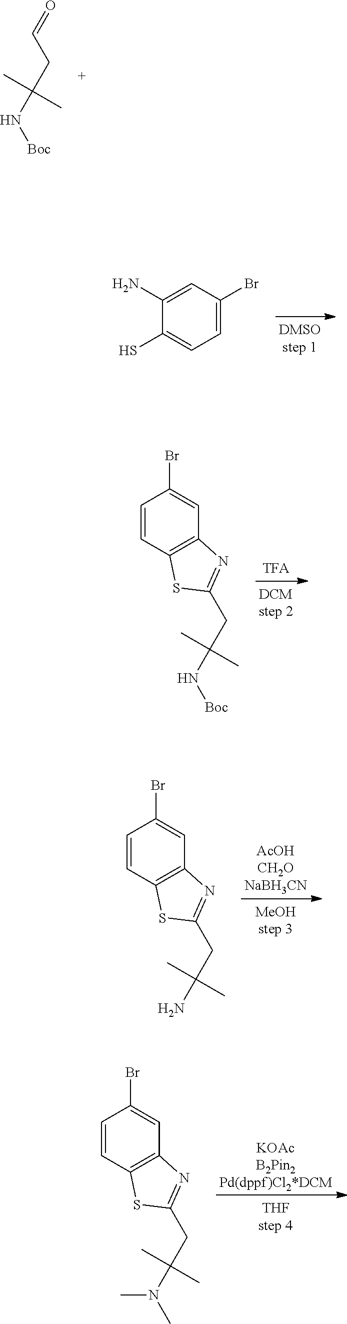

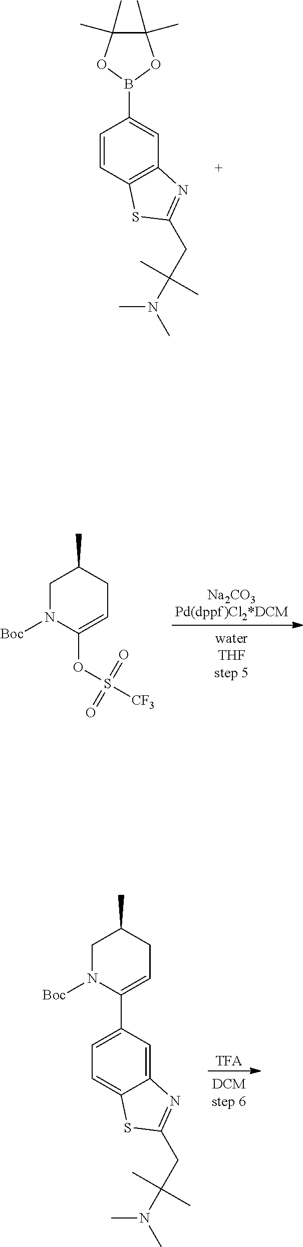 [SMILES: CN(C)C(C)(C)Cc1nc2cc(B3OC(C)(C)C(C)(C)O3)ccc2s1 ; C[C@H]1CC=C(OS(=O)(=O)C(F)(F)F)N(C(=O)OC(C)(C)C)C1 ; "Na2CO3Pd(dppf)Cl2*DCM" ; "TFA" ; C[C@H]1CC=C(c2ccc3sc(CC(C)(C)N(C)C)nc3c2)N(C(=O)OC(C)(C)C)C1]

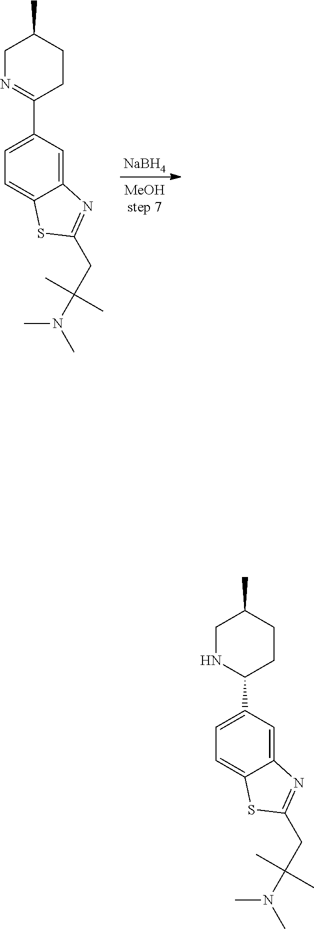 [SMILES: C[C@H]1CCC(c2ccc3sc(CC(C)(C)N(C)C)nc3c2)=NC1 ; "NaBH4" ; C[C@H]1CC[C@H](c2ccc3sc(CC(C)(C)N(C)C)nc3c2)NC1]

### Step 1: Synthesis of tert-butyl (1-(5-bromobenzo[d]thiazol-2-yl)-2-methylpropan-2-yl)carbamate

Prepared by general procedure scheme 4.1 step 1B. Yield: 6.14 g (76.66%).

LCMS(ESI): [M-Boc]⁺ m/z: calcd 285.2; found 286.2; Rt=0.668 min.

### Step 2: Synthesis of 1-(5-bromobenzo[d]thiazol-2-yl)-2-methylpropan-2-amine

tert-Butyl N-[2-(5-bromo-1,3-benzothiazol-2-yl)-1,1-dimethyl-ethyl]carbamate (6.14 g, 15.93 mmol) was dissolved in DCM (20 mL) and TFA (20 mL) was added. The mixture was stirred for 1 hr at 25° C. Then it was evaporated, dissolved in water and Na₂CO₃ was added. The mixture was extracted with DCM twice, the combined organic phase was dried over Na₂SO₄ and concentrated under reduced pressure to obtain 1-(5-bromo-1,3-benzothiazol-2-yl)-2-methyl-propan-2-amine (4.5 g, 15.78 mmol, 99.02% yield).

LCMS(ESI): [M]⁺ m/z: calcd 285.2; found 286.2; Rt=1.010 min.

### Step 3: Synthesis of 1-(5-bromobenzo[d]thiazol-2-yl)-N,N,2-trimethylpropan-2-amine

1-(5-Bromo-1,3-benzothiazol-2-yl)-2-methyl-propan-2-amine (4.5 g, 15.78 mmol) was dissolved in MeOH (40.46 mL) and formaldehyde, 37% in aq. soln., ACS, 36.5-38.0%, stab. with 10-15% MeOH (5.69 g, 189.34 mmol, 5.25 mL), sodium cyan borohydride (2.97 g, 47.33 mmol) and acetic acid (3.79 g, 63.11 mmol, 3.61 mL) was added. The reaction was stirred for 12 hr at 25° C. The reaction mixture was evaporated. Then H₂O was added and it was extracted with DCM twice, combined organic phase was dried over Na₂SO₄ and evaporated to obtain 1-(5-bromo-1,3-benzothiazol-2-yl)-N,N,2-trimethyl-propan-2-amine (4.2 g, 13.41 mmol, 84.98% yield).

LCMS(ESI): [M]⁺ m/z: calcd 313.2; found 314.2; Rt=1.034 min.

### Step 4: Synthesis of N,N,2-trimethyl-1-(5-(4,4,5,5-tetramethyl-1,3,2-dioxaborolan-2-yl)benzo[d]thiazol-2-yl)propan-2-amine

Prepared by general procedure scheme 4.1 step 2. Yield: 4.37 g of crude.

LCMS(ESI): [M]⁺ m/z: calcd 360.2; found 361.2; Rt=1.001 min.

### Step 5: Synthesis of (S)-tert-butyl 6-(2-(2-(dimethylamino)-2-methylpropyl)benzo[d]thiazol-5-yl)-3-methyl-3,4-dihydropyridine-1(2H)-carboxylate

Prepared by general procedure scheme 4.1 step 3. Yield: 7 g of crude.

LCMS(ESI): [M]⁺ m/z: calcd 429.2; found 430.2; Rt=1.040 min.

### Step 6: Synthesis of (S)—N,N,2-trimethyl-1-(5-(5-methyl-3,4,5,6-tetrahydropyridin-2-yl)benzo[d]thiazol-2-yl)propan-2-amine

tert-Butyl (3S)-6-[2-[2-(dimethylamino)-2-methyl-propyl]-1,3-benzothiazol-5-yl]-3-methyl-3,4-dihydro-2H-pyridine-1-carboxylate (7 g, 16.29 mmol) was dissolved in DCM (20 mL) and TFA (20 mL) was added. The mixture was stirred for 1 hr at 25° C. Then it was evaporated, dissolved in water and filtered. To the filtrate Na₂CO₃ was added. The mixture was extracted with DCM twice, the combined organic phase was dried over Na₂SO₄ and concentrated under reduced pressure to obtain N,N,2-trimethyl-1-[5-[(3S)-3-methyl-2,3,4,5-tetrahydropyridin-6-yl]-1,3-benzothiazol-2-yl]propan-2-amine (2.05 g, crude).

LCMS(ESI): [M]⁺ m/z: calcd 329.2; found 330.2; Rt=0.481 min.

### Step 7: Synthesis of N,N,2-trimethyl-1-(5-((2R,5S)-5-methylpiperidin-2-yl)benzo[d]thiazol-2-yl)propan-2-amine

Prepared by general procedure scheme 4.1 step 5. Yield: 1.14 g of crude.

LCMS(ESI): [M]⁺ m/z: calcd 331.2; found 332.2; Rt=0.737 min.

### 3III. 2-((1,3-dimethylpiperidin-4-yl)methyl)-5-((2R,5S)-5-methylpiperidin-2-yl)benzo[d]thiazole

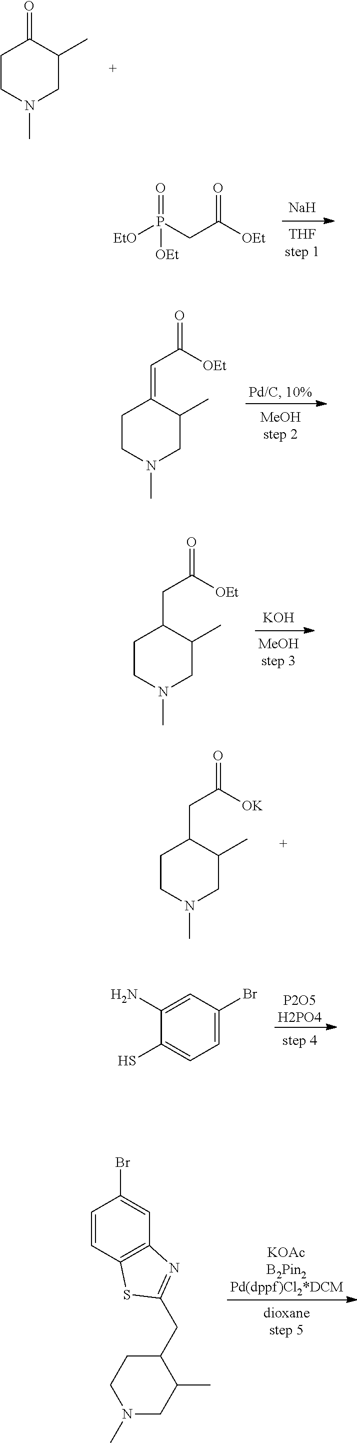

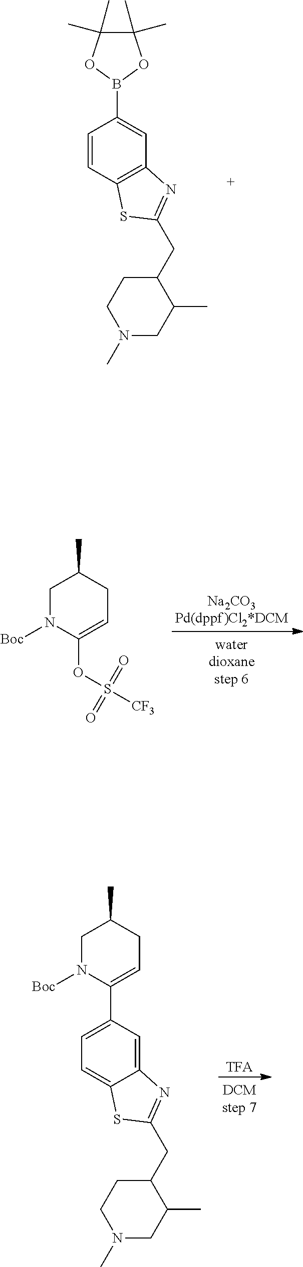

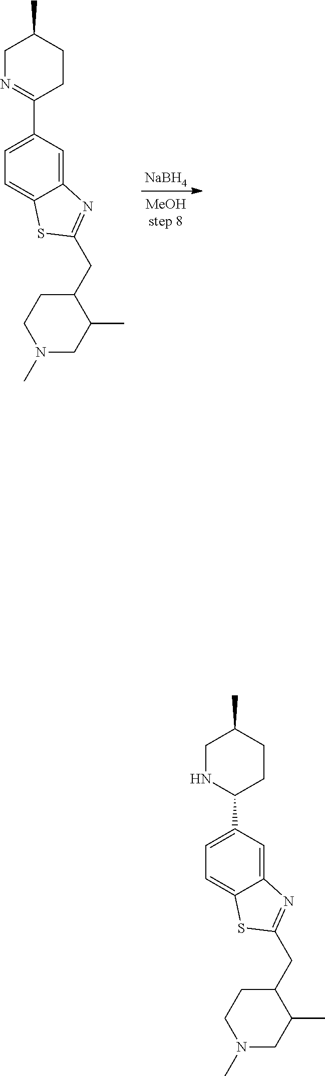 [SMILES: CC1CN(C)CCC1Cc1nc2cc(C3=NC[C@@H](C)CC3)ccc2s1 ; "NaBH4" ; CC1CN(C)CCC1Cc1nc2cc([C@H]3CC[C@H](C)CN3)ccc2s1]

Step 1: Synthesis of (E)-ethyl 2-(1,3-dimethylpiperidin-4-ylidene)acetate

To a stirred solution of sodium hydride (in oil dispersion) 60% dispersion in mineral oil (1.90 g, 82.56 mmol) in THF (300 mL) was added dropwise ethyl 2-(diethoxyphosphoryl)acetate (19.39 g, 86.49 mmol, 17.24 mL), then THF solution of 1,3-dimethylpiperidin-4-one (10 g, 78.63 mmol) was added after 1 hr. The resulting reaction mixture was stirred at 25° C. for 12 hr. Upon completion, the reaction mixture concentrated under reduced pressure. Then it was extracted with MTBE/water (150 ml/30 ml), organic layer was dried over Na₂SO₄ and evaporated. The desired product ethyl (2E)-2-(1,3-dimethyl-4-piperidylidene)acetate (13.5 g, 68.43 mmol, 87.04% yield) was isolated as yellow liquid.

¹H NMR (500 MHz, CDCl₃) δ (ppm) 1.07 (d, 3H), 1.25 (t, 3H), 1.90 (m, 1H), 2.01 (m, 1H), 2.21 (s, 3H), 2.48 (m, 2H), 2.73 (m, 2H), 3.54 (m, 1H), 4.14 (m, 2H), 5.60 (s, 1H).

### Step 2: Synthesis of ethyl 2-(1,3-dimethylpiperidin-4-yl)acetate

To a stirred solution of ethyl (2E)-2-(1,3-dimethyl-4-piperidylidene)acetate (6 g, 30.41 mmol) in MeOH (50 mL) palladium, 10% on carbon (0.5 g, 4.70 mmol) was added at 25° C. The resulting reaction mixture was vacuumed and stirred at 25° C. for 16 hr under hydrogen (61.31 mg, 30.41 mmol). The reaction mixture was filtered off and concentrated in vacuum to give ethyl 2-(1,3-dimethyl-4-piperidyl)acetate (5.48 g, crude).

GCMS: [M]: calcd 199.2; found 199.2; Rt=6.739 min.

### Step 3: Synthesis of potassium 2-(1,3-dimethylpiperidin-4-yl)acetate

To a stirred solution of ethyl 2-(1,3-dimethyl-4-piperidyl)acetate (5.48 g, 27.50 mmol) in MeOH (40.04 mL) were added potassium hydroxide (2.31 g, 41.25 mmol, 1.13 mL) respectively at 25° C. The resulting reaction mixture was stirred at 25° C. for 12 hr, next concentrated in vacuum to give potassium; 2-(1,3-dimethyl-4-piperidyl)acetate (8 g, crude).

GCMS: [M]: calcd 209.2; found 209.2; Rt=2.211 min.

### Step 4: Synthesis of 5-bromo-2-((1,3-dimethylpiperidin-4-yl)methyl)benzo[d]thiazole

To a stirred H₃PO₄ (27.75 g, 240.70 mmol, 15 mL, 85% purity) P₂O₅ (4.5 g, 31.70 mmol) was added at 60° C. and next heated to 120° C. The resulting reaction mixture was stirred at 120° C. for 30 min. Then potassium; 2-(1,3-dimethyl-4-piperidyl)acetate (7.5 g, 21.50 mmol) and 2-amino-4-bromo-benzenethiol (4.39 g, 21.50 mmol) were added and stirred overnight. Upon completion, the reaction mixture was cooled to rt. Then solution of NaOH with ice was added, and quenched to pH=14. Desired product was extracted with EtOAc. Organic layer was washed with water twice, then dried over Na₂SO₄ and evaporated to give 5-bromo-2-[(1,3-dimethyl-4-piperidyl)methyl]-1,3-benzothiazole (5.5 g, crude).

LCMS(ESI): [M]⁺ m/z: calcd 339.2; found 340.2; Rt=0.933 min.

### Step 5: Synthesis of 2-((1,3-dimethylpiperidin-4-yl)methyl)-5-(4,4,5,5-tetramethyl-1,3,2-dioxaborolan-2-yl)benzo[d]thiazole

Prepared by general procedure scheme 4.1 step 2. Yield: 3.8 g of crude.

LCMS(ESI): [M]⁺ m/z: calcd 386.2; found 387.2; Rt=1.010 min.

### Step 6: Synthesis of (3S)-tert-butyl 6-(2-((1,3-dimethylpiperidin-4-yl)methyl)benzo[d]thiazol-5-yl)-3-methyl-3,4-dihydropyridine-1(2H)-carboxylate

Prepared by general procedure scheme 4.1 step 3. Yield: 3.8 g of crude.

LCMS(ESI): [M]⁺ m/z: calcd 455.2; found 456.2; Rt=1.171 min.

### Step 7: Synthesis of 2-((1,3-dimethylpiperidin-4-yl)methyl)-5-((S)-5-methyl-3,4,5,6-tetrahydropyridin-2-yl)benzo[d]thiazole

To a solution of tert-butyl (3S)-6-[2-[(1,3-dimethyl-4-piperidyl)methyl]-1,3-benzothiazol-5-yl]-3-methyl-3,4-dihydro-2H-pyridine-1-carboxylate (3.8 g, 4.17 mmol) in DCM (10 mL) TFA (15 g, 131.55 mmol, 10.14 mL) was added and then stirred for 6 hr at 20° C. The reaction mixture was concentrated in vacuum, treated with water and DCM. Aq. layer was washed with DCM twice. Aq. solution of NaHCO₃ was added to basic pH of solution, desired product was extracted with DCM (2*50 ml). Organic layer was dried over Na₂SO₄ and then concentrated in vacuum to give 2-[(1,3-dimethyl-4-piperidyl)methyl]-5-[(3S)-3-methyl-2,3,4,5-tetrahydropyridin-6-yl]-1,3-benzothiazole (0.8 g, crude).

LCMS(ESI): [M]⁺ m/z: calcd 355.2; found 356.2; Rt=0.542 min.

### Step 8: Synthesis of 2-((1,3-dimethylpiperidin-4-yl)methyl)-5-((2R,5S)-5-methylpiperidin-2-yl)benzo[d]thiazole

Prepared by general procedure scheme 4.1 step 5. Yield: 0.51 g of crude.

LCMS(ESI): [M]⁺ m/z: calcd 357.2; found 358.2; Rt=0.484 min.

### Step 9: Synthesis of 2-((2R,5S)-2-(2-((1,3-dimethylpiperidin-4-yl)methyl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme 4.1 step 6B. Yield: 84.9 mg (26.42%).

HPLC conditions: Column: SunFire C18 100*19 mm, 5 microM; 2-10 min 30-65% MeOH; (loading pump 4 ml/min MeOH).

LCMS(ESI): [M]⁺ m/z: calcd 675.2; found 676.2; Rt=3.129 min.

### 3JJJ. 2-(1,5-dimethyl-1,2,3,6-tetrahydropyridin-4-yl)-5-((2R,5S)-5-methylpiperidin-2-yl)benzo[d]thiazole

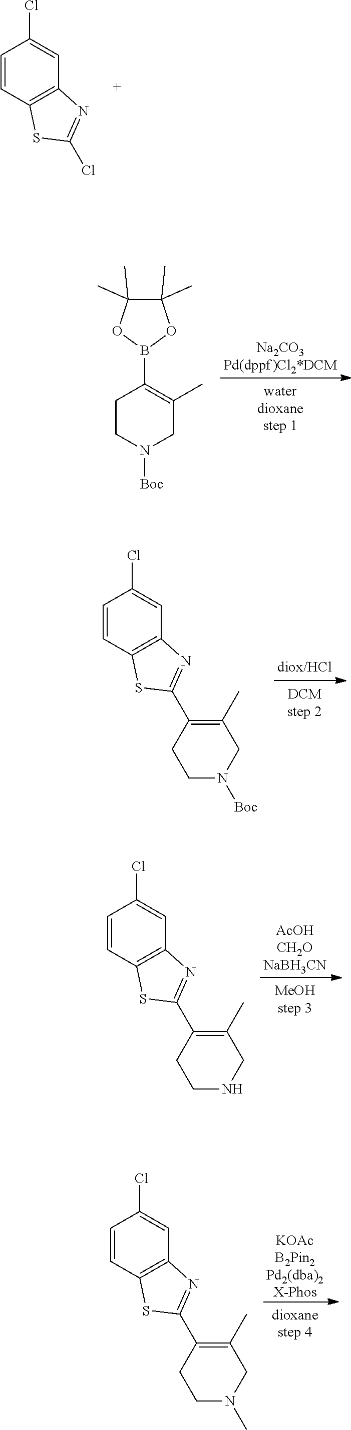

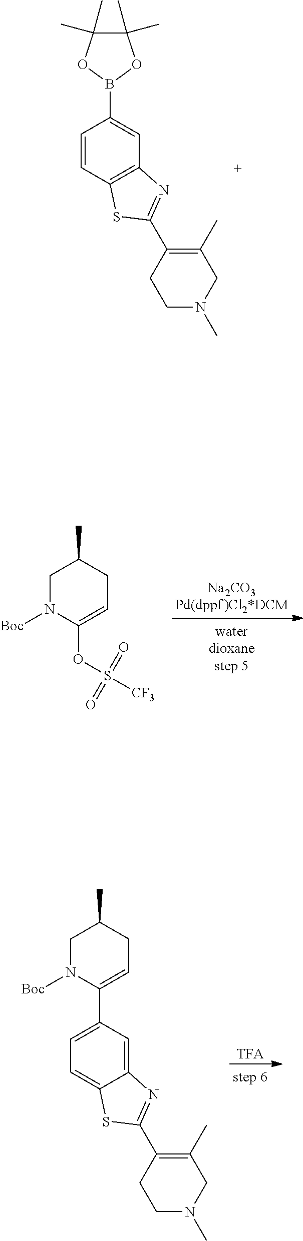

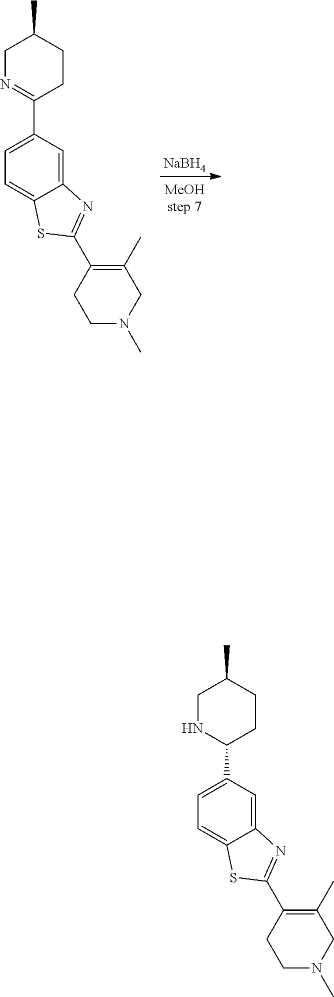 [SMILES: CC1=C(c2nc3cc(C4=NC[C@@H](C)CC4)ccc3s2)CCN(C)C1 ; "NaBH4" ; CC1=C(c2nc3cc([C@H]4CC[C@H](C)CN4)ccc3s2)CCN(C)C1]

### Step 1: Synthesis of tert-butyl 4-(5-chlorobenzo[d]thiazol-2-yl)-3-methyl-5,6-dihydropyridine-1(2H)-carboxylate

Sodium carbonate (19.48 g, 183.75 mmol, 7.69 mL) was added to a solution of 2,5-dichloro-1,3-benzothiazole (15 g, 73.50 mmol) and tert-butyl 5-methyl-4-(4,4,5,5-tetramethyl-1,3,2-dioxaborolan-2-yl)-3,6-dihydro-2H-pyridine-1-carboxylate (23.76 g, 73.50 mmol) in dioxane (200 mL) and water (65 mL). Reaction flask was evacuated and refilled with argon 3 times. Then, Pd(dppf)Cl₂*DCM (3.00 g, 3.68 mmol) was added under stream of argon. Resulting mixture was stirred at 95° C. for 15 hr under inert atmosphere. Reaction solution was decanted and concentrated under reduce pressure to give crude product which was diluted with MTBE (300 ml). The resulting cloudy solution was decanted from oily residue. MTBE was evaporated in vacuum to give tert-butyl 4-(5-chloro-1,3-benzothiazol-2-yl)-5-methyl-3,6-dihydro-2H-pyridine-1-carboxylate (18 g, 49.33 mmol, 67.11% yield).

LCMS(ESI): [M]⁺ m/z: calcd 364.2; found 365.2; Rt=1.802 min.

### Step 2: Synthesis of 5-chloro-2-(5-methyl-1,2,3,6-tetrahydropyridin-4-yl)benzo[d]thiazole

To the stirred solution of tert-butyl 4-(5-chloro-1,3-benzothiazol-2-yl)-5-methyl-3,6-dihydro-2H-pyridine-1-carboxylate (18 g, 49.33 mmol) in DCM (150 mL) hydrogen chloride solution 4.0M in dioxane (80.00 g, 2.19 mol, 100 mL) was added. The resulting mixture was stirred at 25° C. for 14 hr. Solvents were evaporated in vacuum. The residue was diluted with MTBE (200 ml). Solid was filtered, washed with MTBE, then dried in vacuum to give 5-chloro-2-(5-methyl-1,2,3,6-tetrahydropyridin-4-yl)-1,3-benzothiazole (8.5 g, 28.22 mmol, 57.20% yield, HCl).

¹H NMR (500 MHz, DMSO-d₆) δ (ppm) 2.12 (s, 3H), 2.85 (m, 2H), 3.26 (m, 2H), 3.75 (m, 2H), 7.49 (d, 1H), 8.08 (s, 1H), 8.16 (d, 1H), 9.73 (bds, 2H).

### Step 3: Synthesis of 5-chloro-2-(1,5-dimethyl-1,2,3,6-tetrahydropyridin-4-yl)benzo[d]thiazole

To the stirred solution of 5-chloro-2-(5-methyl-1,2,3,6-tetrahydropyridin-4-yl)-1,3-benzothiazole (8.5 g, 28.22 mmol, HCl) in MeOH (300 mL) formaldehyde, 37% w/w aq. soln., stab. with 7-8% MeOH (3.43 g, 42.33 mmol, 3.17 mL, 37% purity) and sodium acetate, anhydrous (5.79 g, 70.54 mmol, 3.79 mL) were added. The resulting mixture was stirred for 2 hr at 25° C. Then Sodium cyan borohydride (1.95 g, 31.04 mmol) was added portion wise. The resulting mixture was stirred at 25° C. for 12 hr. MeOH was evaporated. The residue was diluted with 10% aqueous sodium hydroxide solution (150 ml) and extracted with DCM (3*100 ml). The combined organic extracts were dried over sodium sulphate and concentrated in vacuum to afford crude 5-chloro-2-(1,5-dimethyl-3,6-dihydro-2H-pyridin-4-yl)-1,3-benzothiazole (6.1 g, 21.88 mmol, 77.54% yield).

LCMS(ESI): [M]⁺ m/z: calcd 278.2; found 279.2; Rt=1.006 min.

### Step 4: Synthesis of 2-(1,5-dimethyl-1,2,3,6-tetrahydropyridin-4-yl)-5-(4,4,5,5-tetramethyl-1,3,2-dioxaborolan-2-yl)benzo[d]thiazole

5-Chloro-2-(1,5-dimethyl-3,6-dihydro-2H-pyridin-4-yl)-1,3-benzothiazole (6.2 g, 22.24 mmol), 4,4,5,5-tetramethyl-2-(4,4,5,5-tetramethyl-1,3,2-dioxaborolan-2-yl)-1,3,2-dioxaborolane (5.65 g, 22.24 mmol) and potassium acetate (4.37 g, 44.48 mmol, 2.78 mL) were mixed in dioxane (70 mL). The resulting mixture was evacuated and then backfilled with argon, this operation was repeated three times, then tris(dibenzylideneacetone)dipalladium (0) (1.02 g, 1.11 mmol) and XPhos (1.06 g, 2.22 mmol) were added under argon. The reaction mixture was stirred under argon at 90° C. for 15 hr. The reaction mixture was cooled down and filtered. The filter cake was washed with dioxane (2*10 ml) and discarded. The combined filtrate was concentrated in vacuum. The residue was diluted with MTBE (100 ml) and extracted with a NaHSO₄ water solution (30 ml) (repeated 3 times). The combined aqueous layer was basified to pH 10 with 10% aqueous sodium hydroxide to give water solution of 2-(1,5-dimethyl-3,6-dihydro-2H-pyridin-4-yl)-5-(4,4,5,5-tetramethyl-1,3,2-dioxaborolan-2-yl)-1,3-benzothiazole (8 g, 21.60 mmol, 97.14% yield) which was used directly in the next step.

LCMS(ESI): [M]⁺ m/z: calcd 370.2; found 371.2; Rt=1.178 min.

### Step 5: Synthesis of (S)-tert-butyl 6-(2-(1,5-dimethyl-1,2,3,6-tetrahydropyridin-4-yl)benzo[d]thiazol-5-yl)-3-methyl-3,4-dihydropyridine-1(2H)-carboxylate

Prepared by general procedure scheme 4.1 step 3. Yield: 6 g of crude.

LCMS(ESI): [M]⁺ m/z: calcd 439.2; found 440.2; Rt=1.141 min.

### Step 6: Synthesis of (S)-2-(1,5-dimethyl-1,2,3,6-tetrahydropyridin-4-yl)-5-(5-methyl-3,4,5,6-tetrahydropyridin-2-yl)benzo[d]thiazole

tert-Butyl (3S)-6-[2-(1,5-dimethyl-3,6-dihydro-2H-pyridin-4-yl)-1,3-benzothiazol-5-yl]-3-methyl-3,4-dihydro-2H-pyridine-1-carboxylate (6 g, 13.65 mmol) was diluted with TFA (88.80 g, 778.79 mmol, 60.00 mL). The resulting mixture was stirred at 25° C. for 1.5 hr. TFA was evaporated in vacuum. The residue was diluted with water (150 ml). The resulting cloudy solution was decanted from oily residue, and then basified with NaHCO₃. Product was extracted with DCM (3*50 ml). Combined organic layers were dried over Na₂SO₄. DCM was evaporated in vacuum to give 2-(1,5-dimethyl-3,6-dihydro-2H-pyridin-4-yl)-5-[(3S)-3-methyl-2,3,4,5-tetrahydropyridin-6-yl]-1,3-benzothiazole (3.2 g, 9.43 mmol, 69.06% yield).

LCMS(ESI): [M]⁺ m/z: calcd 339.2; found 340.2; Rt=0.583 min.

### Step 7: Synthesis of 2-(1,5-dimethyl-1,2,3,6-tetrahydropyridin-4-yl)-5-((2R,5S)-5-methylpiperidin-2-yl)benzo[d]thiazole

Prepared by general procedure scheme 4.1 step 5. Yield: 2.3 g of crude.

LCMS(ESI): [M]⁺ m/z: calcd 341.2; found 342.2; Rt=0.628 min.

### 3JJJ. 2-(rac-(3S,4R)-3-methoxy-1-methylpiperidin-4-yl)-5-((2R,5S)-5-methylpiperidin-2-yl)benzo[d]thiazole

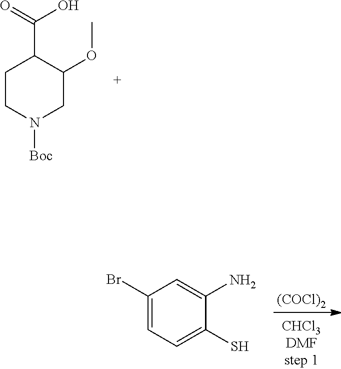 [SMILES: COC1CN(C(=O)OC(C)(C)C)CCC1C(=O)O ; Nc1cc(Br)ccc1S ; "(COCl)2"]

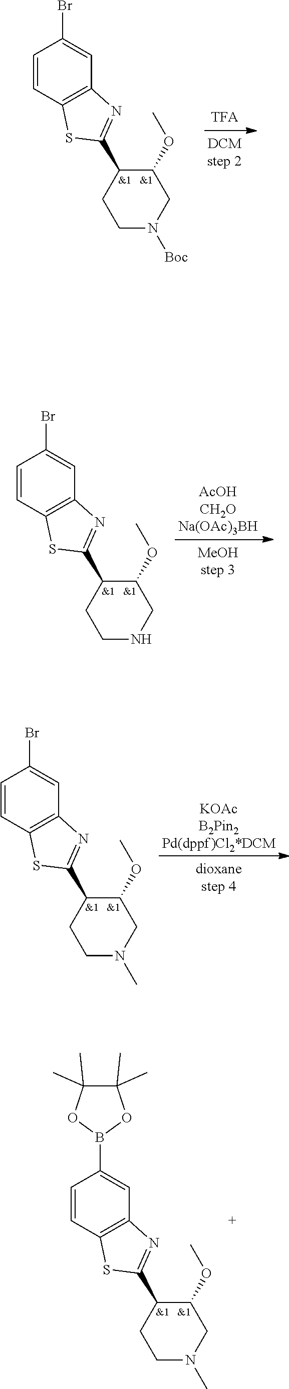

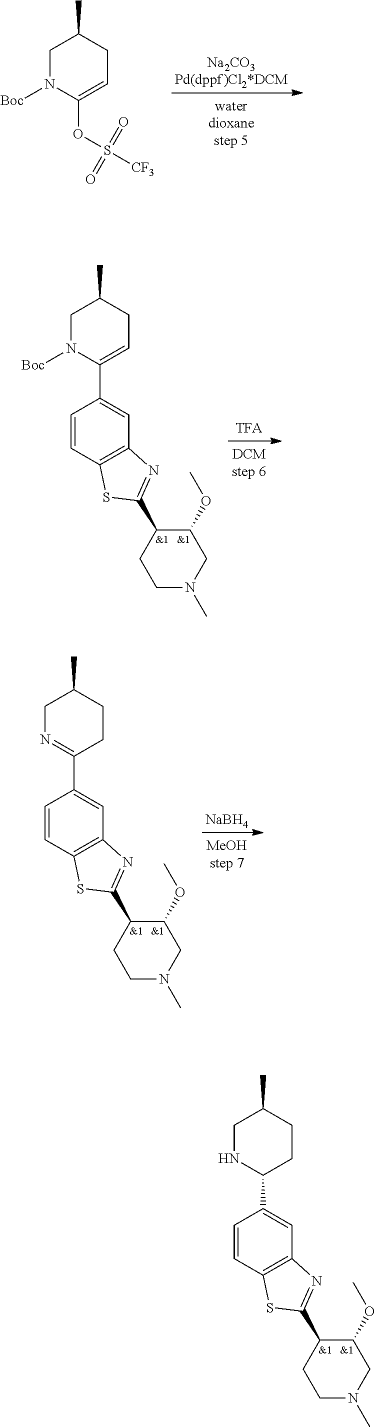

### Step 1: Synthesis of rac-(3S,4R)-tert-butyl 4-(5-bromobenzo[d]thiazol-2-yl)-3-methoxypiperidine-1-carboxylate

Oxalyl chloride (1.08 g, 8.48 mmol, 740.16 μL) was added in one portion to a stirred slurry of 1-tert-butoxycarbonyl-3-methoxy-piperidine-4-carboxylic acid (2 g, 7.71 mmol) in CHCl₃ (60 mL). After few minutes, DMF (0.1 mL) (catalytic amount) was added and the resulting mixture was stirred at 25° C. until clear solution formed and gaseous products evolution had stopped (approximately 1 hr), then 2-amino-4-bromo-benzenethiol (1.57 g, 7.71 mmol) was added in one portion under argon. The resulting slurry was stirred vigorously at 25° C. for 18 hr, and then the resulting heavy slurry was neutralized with 10% aqueous sodium carbonate solution. The organic layer was separated, dried over sodium sulfate and concentrated in vacuum to afford crude product 2.2 g (50% purity by LCMS), which was purified by column chromatography on silica using hexane/MTBE gradient (0-17% of MTBE) to afford tert-butyl rac-(3S,4R)-4-(5-bromo-1,3-benzothiazol-2-yl)-3-methoxy-piperidine-1-carboxylate (1 g, 2.34 mmol, 30.34% yield) as light-yellow solid.

LCMS(ESI): [M]⁺ m/z: calcd 427.2; found 428.2; Rt=1.652 min.

### Step 2: Synthesis of rac-5-bromo-2-((3S,4R)-3-methoxypiperidin-4-yl)benzo[d]thiazole

TFA (7.40 g, 64.90 mmol, 5 mL) was added in one portion to a stirred solution of tert-butyl rac-(3S,4R)-4-(5-bromo-1,3-benzothiazol-2-yl)-3-methoxy-piperidine-1-carboxylate (1 g, 2.34 mmol) in DCM (5 mL). The resulting solution was stirred at 25° C. for 0.5 hr, and then concentrated in vacuum. The residue was diluted with water (10 ml) and basified to pH 10-11 with 10% aqueous sodium carbonate solution and then extracted with DCM (2*15 ml). The combined organic extracts were dried over sodium sulfate and concentrated in vacuum to afford 5-bromo-2-[rac-(3S,4R)-3-methoxy-4-piperidyl]-1,3-benzothiazole (650 mg, 1.99 mmol, 84.89% yield) as light-yellow gum, which was directly used in the next step.

LCMS(ESI): [M]⁺ m/z: calcd 327.2; found 328.2; Rt=0.975 min.

### Step 3: Synthesis of rac-5-bromo-2-((3S,4R)-3-methoxy-1-methylpiperidin-4-yl)benzo[d]thiazole

Formaldehyde, 37% w/w aq. soln., stab. with 7-8% MeOH (241.82 mg, 2.98 mmol, 223.29 μL, 37% purity) and acetic acid (238.56 mg, 3.97 mmol, 227.41 μL) were added to a stirred solution of 5-bromo-2-[rac-(3S,4R)-3-methoxy-4-piperidyl]-1,3-benzothiazole (650 mg, 1.99 mmol) in MeOH (20 mL) at 25° C. The resulting mixture was stirred at 25° C. for 1 hr, then sodium cyan borohydride (149.78 mg, 2.38 mmol) was added in one portion at 25° C. (foaming!). The reaction mixture was stirred at 25° C. for 18 hr, and then concentrated in vacuum. The residue was diluted with 10% aqueous sodium hydroxide solution (50 ml) and extracted with DCM (2*30 ml). The combined organic extracts were dried over sodium sulphate and concentrated in vacuum to afford crude 5-bromo-2-[rac-(3S,4R)-3-methoxy-1-methyl-4-piperidyl]-1,3-benzothiazole (600 mg, 1.76 mmol, 88.51% yield) as light-brown gum, which was used directly in the next step.

LCMS(ESI): [M]⁺ m/z: calcd 341.2; found 342.2; Rt=0.750 min.

### Step 4: Synthesis of rac-2-((3S,4R)-3-methoxy-1-methylpiperidin-4-yl)-5-(4,4,5,5-tetramethyl-1,3,2-dioxaborolan-2-yl)benzo[d]thiazole

Prepared by general procedure scheme 4.1 step 2. Yield: 680 mg of crude.

LCMS(ESI): [M]⁺ m/z: calcd 388.2; found 389.2; Rt=1.018 min.

### Step 5: Synthesis of (S)-tert-butyl 6-(2-(rac-(3S,4R)-3-methoxy-1-methylpiperidin-4-yl)benzo[d]thiazol-5-yl)-3-methyl-3,4-dihydropyridine-1(2H)-carboxylate

Prepared by general procedure scheme 4.1 step 3. Yield: 1.5 g of crude.

LCMS(ESI): [M]⁺ m/z: calcd 457.2; found 458.2; Rt=1.241 min.

### Step 6: Synthesis of 2-(rac-(3S,4R)-3-methoxy-1-methylpiperidin-4-yl)-5-((S)-5-methyl-3,4,5,6-tetrahydropyridin-2-yl)benzo[d]thiazole

Prepared by general procedure scheme 4.1 step 4. Yield: 0.4 g of crude.

LCMS(ESI): [M]⁺ m/z: calcd 357.2; found 358.2; Rt=0.668 min.

### Step 7: Synthesis of 2-(rac-(3S,4R)-3-methoxy-1-methylpiperidin-4-yl)-5-((2R,5S)-5-methylpiperidin-2-yl)benzo[d]thiazole

Prepared by general procedure scheme 4.1 step 5. Yield: 340 mg of crude.

LCMS(ESI): [M]⁺ m/z: calcd 359.2; found 360.2; Rt=0.721 min.

### 3JJJ. 2-(1,4-dimethylpiperidin-4-yl)-5-((2R,5S)-5-methylpiperidin-2-yl)benzo[d]thiazole

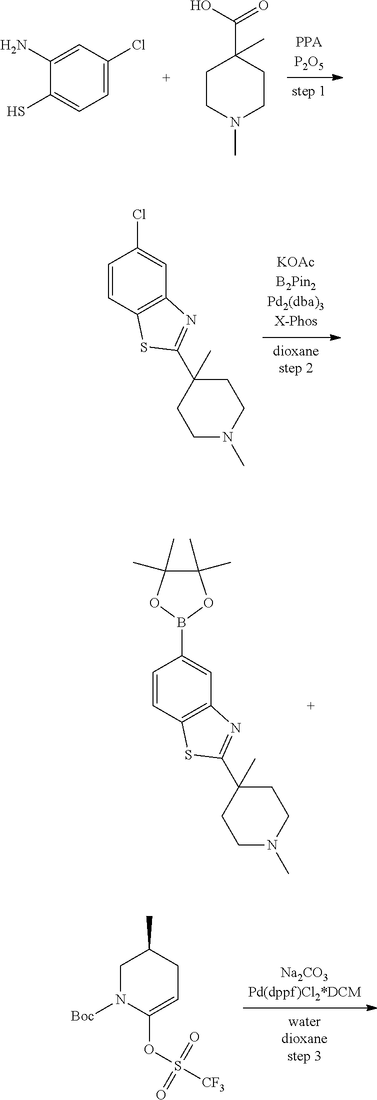

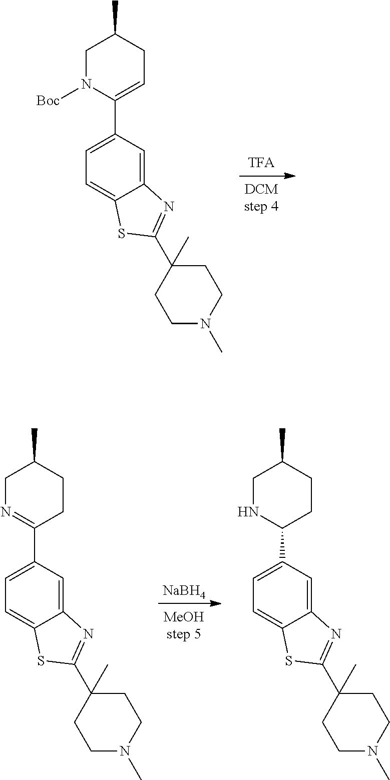 [SMILES: C[C@H]1CC=C(c2ccc3sc(C4(C)CCN(C)CC4)nc3c2)N(C(=O)OC(C)(C)C)C1 ; C[C@H]1CCC(c2ccc3sc(C4(C)CCN(C)CC4)nc3c2)=NC1 ; "NaBH4" ; C[C@H]1CC[C@H](c2ccc3sc(C4(C)CCN(C)CC4)nc3c2)NC1]

### Step 1: Synthesis of 5-chloro-2-(1,4-dimethylpiperidin-4-yl)benzo[d]thiazoleamine

Prepared by general procedure scheme 4.1 step 1A. Yield: 3 g (41.38%).

CC conditions: The crude product was purified by silica gel with MeCN/MeOH (gradient 10-100% MeOH) as an eluent mixture.

LCMS(ESI): [M]⁺ m/z: calcd 280.2; found 281.2; Rt=1.529 min.

### Step 2: Synthesis of 2-(1,4-dimethylpiperidin-4-yl)-5-(4,4,5,5-tetramethyl-1,3,2-dioxaborolan-2-yl)benzo[d]thiazole

tris(Dibenzylideneacetone)dipalladium(0) (652.18 mg, 712.21 mol) and XPhos (1.36 g, 2.85 mmol) was added to a solution of 5-chloro-2-(1,4-dimethyl-4-piperidyl)-1,3-benzothiazole (4 g, 14.24 mmol) and 4,4,5,5-tetramethyl-2-(4,4,5,5-tetramethyl-1,3,2-dioxaborolan-2-yl)-1,3,2-dioxaborolane (4.70 g, 18.52 mmol) in dioxane (60.00 mL). Reaction flask was evacuated and refilled with argon 3 times. Then potassium acetate (2.80 g, 28.49 mmol, 1.78 mL) was added under stream of argon. Resulting mixture was stirred at 100° C. for 15 hr under inert atmosphere, then cooled and evaporated in vacuum poured into water (120 ml) and extracted with DCM (2×50 ml), dried over sodium sulphate and evaporated in vacuum to afford 2-(1,4-dimethyl-4-piperidyl)-5-(4,4,5,5-tetramethyl-1,3,2-dioxaborolan-2-yl)-1,3-benzothiazole (0.9 g, 2.42 mmol, 16.97% yield).

LCMS(ESI): [M]⁺ m/z: calcd 372.2; found 373.2; Rt=3.119 min.

### Step 3: Synthesis of (S)-tert-butyl 6-(2-(1,4-dimethylpiperidin-4-yl)benzo[d]thiazol-5-yl)-3-methyl-3,4-dihydropyridine-1(2H)-carboxylate

Prepared by general procedure scheme 4.1 step 3. Yield: 1.3 g of crude.

LCMS(ESI): [M]⁺ m/z: calcd 441.2; found 442.2; Rt=1.289 min.

### Step 4: Synthesis of (S)-2-(1,4-dimethylpiperidin-4-yl)-5-(5-methyl-3,4,5,6-tetrahydropyridin-2-yl)benzo[d]thiazole

Prepared by general procedure scheme 4.1 step 4. Yield: 0.7 g of crude.

LCMS(ESI): [M]⁺ m/z: calcd 341.2; found 342.2; Rt=0.724 min.

### Step 5: Synthesis of 2-(1,4-dimethylpiperidin-4-yl)-5-((2R,5S)-5-methylpiperidin-2-yl)benzo[d]thiazole

Prepared by general procedure scheme 4.1 step 5. Yield: 0.55 g of crude.

LCMS(ESI): [M]⁺ m/z: calcd 343.2; found 344.2; Rt=1.772 min.

### 3KKK. 2-(1-azabicyclo[2.2.1]heptan-4-yl)-5-((2R,5S)-5-methylpiperidin-2-yl)benzo[d]thiazole

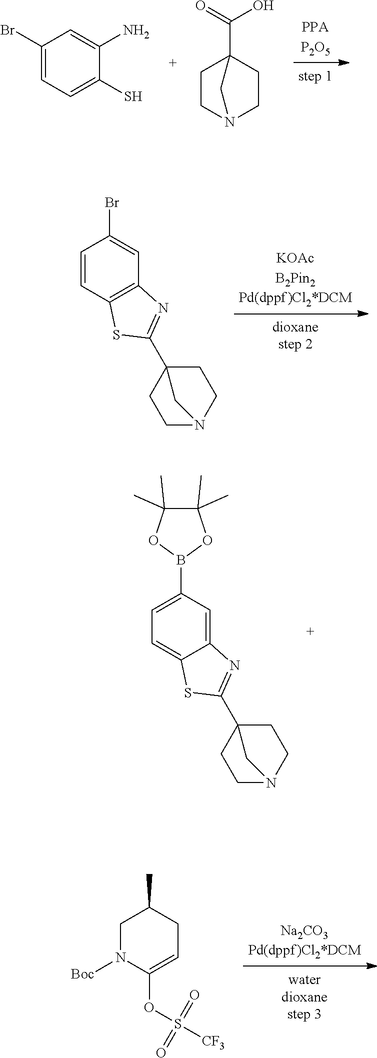 [SMILES: Nc1cc(Br)ccc1S ; O=C(O)C12CCN(CC1)C2 ; Brc1ccc2sc(C34CCN(CC3)C4)nc2c1 ; CC1(C)OB(c2ccc3sc(C45CCN(CC4)C5)nc3c2)OC1(C)C ; C[C@H]1CC=C(OS(=O)(=O)C(F)(F)F)N(C(=O)OC(C)(C)C)C1]

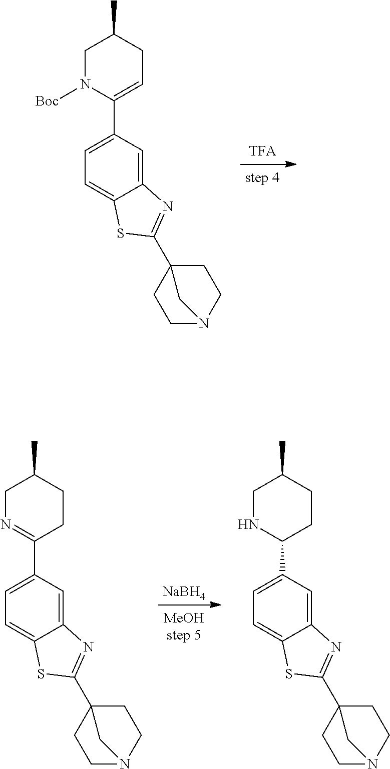 [SMILES: C[C@H]1CC=C(c2ccc3sc(C45CCN(CC4)C5)nc3c2)N(C(=O)OC(C)(C)C)C1 ; C[C@H]1CCC(c2ccc3sc(C45CCN(CC4)C5)nc3c2)=NC1 ; "NaBH4" ; C[C@H]1CC[C@H](c2ccc3sc(C45CCN(CC4)C5)nc3c2)NC1]

### Step 1: Synthesis of 2-(1-azabicyclo[2.2.1]heptan-4-yl)-5-bromobenzo[d]thiazole

Prepared by general procedure scheme 4.1 step 1A. Yield: 3.5 g (92.4%).

LCMS(ESI): [M]⁺ m/z: calcd 309.2; found 310.2; Rt=0.961 min.

### Step 2: Synthesis of 2-(1-azabicyclo[2.2.1]heptan-4-yl)-5-(4,4,5,5-tetramethyl-1,3,2-dioxaborolan-2-yl)benzo[d]thiazole

Prepared by general procedure scheme 4.1 step 2. Yield: 4 g of crude.

LCMS(ESI): [M]⁺ m/z: calcd 356.2; found 357.2; Rt=1.025 min.

### Step 3: Synthesis of (S)-tert-butyl 6-(2-(1-azabicyclo[2.2.1]heptan-4-yl)benzo[d]thiazol-5-yl)-3-methyl-3,4-dihydropyridine-1(2H)-carboxylate

Prepared by general procedure scheme 4.1 step 3. Yield: 8 g of crude.

LCMS(ESI): [M]⁺ m/z: calcd 425.2; found 426.2; Rt=1.254 min.

### Step 4: Synthesis of (S)-2-(1-azabicyclo[2.2.1]heptan-4-yl)-5-(5-methyl-3,4,5,6-tetrahydropyridin-2-yl)benzo[d]thiazole)

Prepared by general procedure scheme 4.1 step 4. Yield: 6 g of crude.

LCMS(ESI): [M]⁺ m/z: calcd 325.2; found 326.2; Rt=0.683 min.

### Step 5: Synthesis of 2-(1-azabicyclo[2.2.1]heptan-4-yl)-5-((2R,5S)-5-methylpiperidin-2-yl)benzo[d]thiazole

Prepared by general procedure scheme 4.1 step 5. Yield: 2 g of crude.

LCMS(ESI): [M]⁺ m/z: calcd 327.2; found 328.2; Rt=0.709 min.

### 3LLL. N,N-dimethyl-3-(5-((2R,5S)-5-methylpiperidin-2-yl)benzo[d]thiazol-2-yl)cyclobutanamine

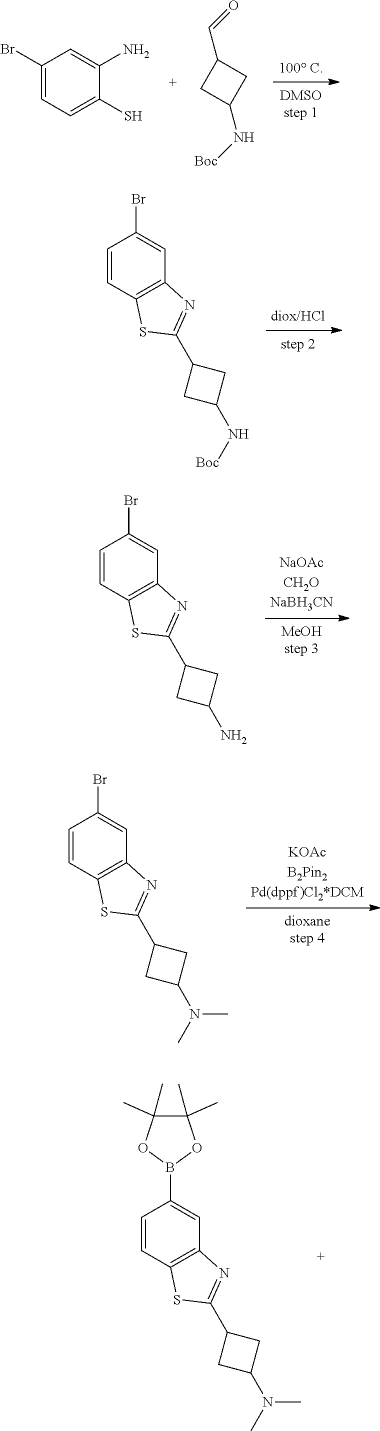

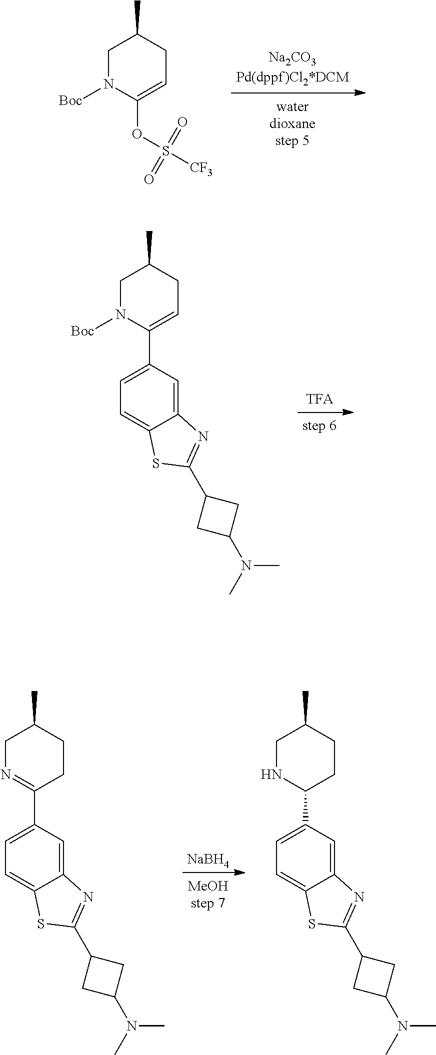 [SMILES: C[C@H]1CC=C(OS(=O)(=O)C(F)(F)F)N(C(=O)OC(C)(C)C)C1 ; C[C@H]1CC=C(c2ccc3sc(C4CC(N(C)C)C4)nc3c2)N(C(=O)OC(C)(C)C)C1 ; C[C@H]1CCC(c2ccc3sc(C4CC(N(C)C)C4)nc3c2)=NC1 ; "NaBH4" ; C[C@H]1CC[C@H](c2ccc3sc(C4CC(N(C)C)C4)nc3c2)NC1]

### Step 1: Synthesis of tert-butyl (3-(5-bromobenzo[d]thiazol-2-yl)cyclobutyl)carbamate

Prepared by general procedure scheme 4.1 step 1B. Yield: 5 g (53.24%).

LCMS(ESI): [M]⁺ m/z: calcd 383.2; found 384.2; Rt=1.402 min.

### Step 2: Synthesis of 3-(5-bromobenzo[d]thiazol-2-yl)cyclobutanamine

tert-Butyl N-[3-(5-bromo-1,3-benzothiazol-2-yl)cyclobutyl]carbamate (5 g, 13.04 mmol) was treated with hydrogen chloride solution 4.0M in dioxane (24.00 g, 658.26 mmol, 30 mL). The resulting mixture was stirred at 25° C. for 14 hr. Precipitate was filtered and additionally washed with MTBE. Then dried in vacuum to give 3-(5-bromo-1,3-benzothiazol-2-yl)cyclobutanamine (4 g, 12.51 mmol, 95.93% yield, HCl).

LCMS(ESI): [M]⁺ m/z: calcd 284.2; found 285.2; Rt=0.931 min.

### Step 3: Synthesis of 3-(5-bromobenzo[d]thiazol-2-yl)-N,N-dimethylcyclobutanamine

To the stirred solution of 3-(5-bromo-1,3-benzothiazol-2-yl)cyclobutanamine (4 g, 12.51 mmol, HCl) in MeOH (71.00 mL) formaldehyde, 37% w/w aq. soln., stab. with 7-8% MeOH (2.54 g, 31.28 mmol, 2.34 mL, 37% purity) and sodium acetate, anhydrous (2.57 g, 31.28 mmol, 1.68 mL) were added. The resulting mixture was stirred for 2 hr at 25° C. Then Sodium cyan borohydride (1.57 g, 25.03 mmol) was added portion wise. The resulting mixture was stirred at 25° C. for 12 hr. MeOH was evaporated. The residue was diluted with water (100 ml) and extracted with DCM (3*50 ml). Combined organic layers were dried over Na₂SO₄. DCM was evaporated in vacuum to give 3-(5-bromo-1,3-benzothiazol-2-yl)-N,N-dimethyl-cyclobutanamine (3.9 g, crude).

LCMS(ESI): [M]⁺ m/z: calcd 311.2; found 312.2; Rt=0.829 min.

### Step 4: Synthesis of N,N-dimethyl-3-(5-(4,4,5,5-tetramethyl-1,3,2-dioxaborolan-2-yl)benzo[d]thiazol-2-yl)cyclobutanamine

Prepared by general procedure scheme 4.1 step 2. Yield: 4 g of crude.

LCMS(ESI): [M]⁺ m/z: calcd 358.2; found 359.2; Rt=1.029 min.

### Step 5: Synthesis of (S)-tert-butyl 6-(2-(3-(dimethylamino)cyclobutyl)benzo[d]thiazol-5-yl)-3-methyl-3,4-dihydropyridine-1(2H)-carboxylate

Prepared by general procedure scheme 4.1 step 3. Yield: 7 g of crude.

LCMS(ESI): [M]⁺ m/z: calcd 427.2; found 428.2; Rt=1.057 min.

### Step 6: Synthesis of (S)—N,N-dimethyl-3-(5-(5-methyl-3,4,5,6-tetrahydropyridin-2-yl)benzo[d]thiazol-2-yl)cyclobutanamine

Prepared by general procedure scheme 4.1 step 4. Yield: 3 g of crude.

LCMS(ESI): [M]⁺ m/z: calcd 327.2; found 328.2; Rt=0.635 min.

### Step 7: Synthesis of N,N-dimethyl-3-(5-((2R,5S)-5-methylpiperidin-2-yl)benzo[d]thiazol-2-yl)cyclobutanamine

Prepared by general procedure scheme 4.1 step 5. Yield: 80 mg (2.65%).

HPLC conditions: Column: Chromatorex C18 100*19 mm, 5 microM; 0-1-6 min 45-45-85% water-MeOH, flow: 30 ml/min; (loading pump 4 ml/min MeOH).

LCMS(ESI): [M]⁺ m/z: calcd 329.2; found 330.2; Rt=1.324 min.

### 3MMM. 2-(2-methyl-2-azabicyclo[2.2.2]octan-4-yl)-5-((2R,5S)-5-methylpiperidin-2-yl)benzo[d]thiazole

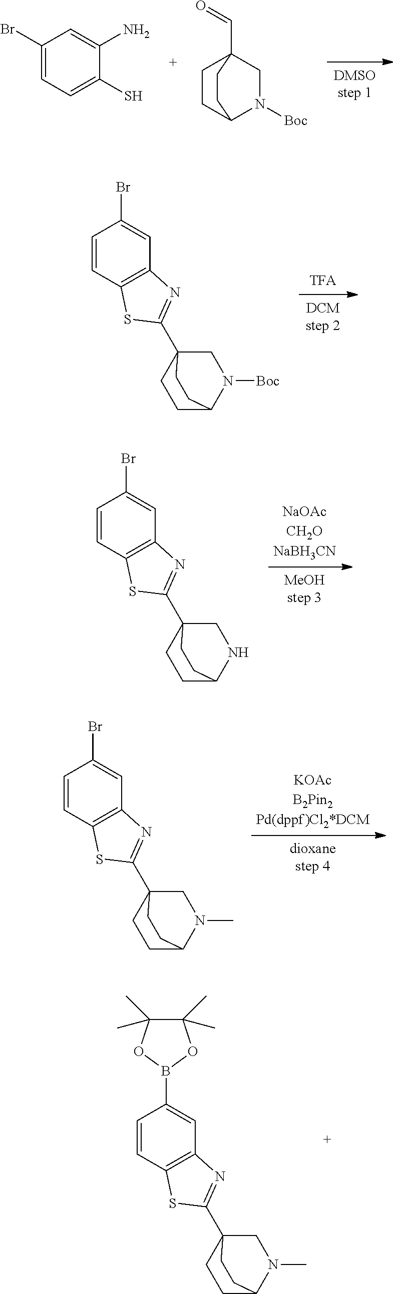

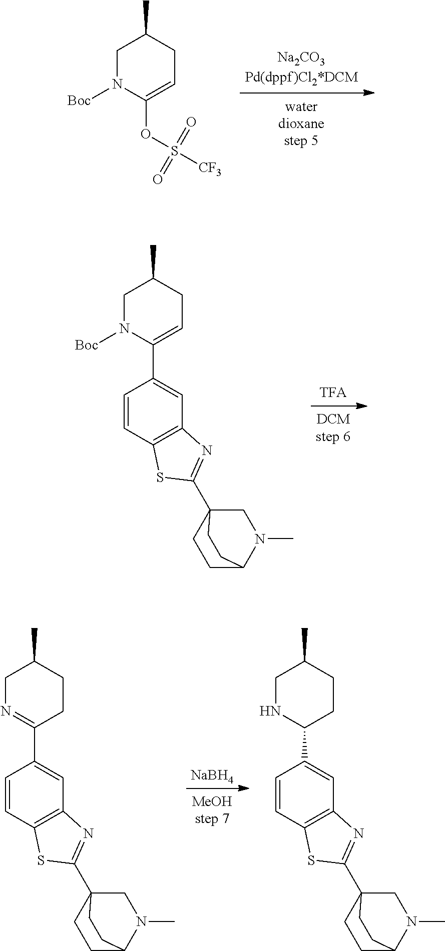 [SMILES: C[C@H]1CC=C(OS(=O)(=O)C(F)(F)F)N(C(=O)OC(C)(C)C)C1 ; C[C@H]1CC=C(c2ccc3sc(C45CCC(CC4)N(C)C5)nc3c2)N(C(=O)OC(C)(C)C)C1 ; C[C@H]1CCC(c2ccc3sc(C45CCC(CC4)N(C)C5)nc3c2)=NC1 ; "NaBH4" ; C[C@H]1CC[C@H](c2ccc3sc(C45CCC(CC4)N(C)C5)nc3c2)NC1]

### Step 1: Synthesis of tert-butyl 4-(5-bromobenzo[d]thiazol-2-yl)-2-azabicyclo[2.2.2]octane-2-carboxylate

Prepared by general procedure scheme 4.1 step 1B. Yield: 2.82 g (79.7%).

LCMS(ESI): [M]⁺ m/z: calcd 423.2; found 424.2; Rt=1.572 min.

### Step 2: Synthesis of 2-(2-azabicyclo[2.2.2]octan-4-yl)-5-bromobenzo[d]thiazole

TFA (7.56 g, 66.29 mmol, 5.11 mL) was added to a solution of tert-butyl 4-(5-bromo-3a,7a-dihydro-1,3-benzothiazol-2-yl)-2-azabicyclo[2.2.2]octane-2-carboxylate (2.82 g, 6.63 mmol) in DCM (20 mL). Resulting mixture was stirred at 20° C. for 4 hr. Then, volatiles were removed under reduced pressure, leaving 2-(2-azabicyclo[2.2.2]octan-4-yl)-5-bromo-1,3-benzothiazole (3 g, crude, TFA).

LCMS(ESI): [M]⁺ m/z: calcd 323.2; found 324.2; Rt=1.025 min.

### Step 3: Synthesis of 5-bromo-2-(2-methyl-2-azabicyclo[2.2.2]octan-4-yl)benzo[d]thiazole

Formaldehyde, 37% w/w aq. soln., stab. with 7-8% MeOH (1.12 g, 13.75 mmol, 1.03 mL, 37% purity) and sodium acetate (1.69 g, 20.63 mmol, 1.11 mL) were added to the solution of 2-(2-azabicyclo[2.2.2]octan-4-yl)-5-bromo-1,3-benzothiazole (3 g, 6.88 mmol, TFA) in MeOH (50 mL). Resulting mixture was stirred at 20° C. for 1 hr before sodium cyan borohydride (864.27 mg, 13.75 mmol) was added thereto. After that, stirring was continued for 18 hr. Then, solvent was removed under reduced pressure and residue was partitioned between 15% aq. K₂CO₃ solution (30 ml) and DCM (50 ml). Organic layer was separated, dried over solid K₂CO₃ and concentrated under reduced pressure, affording 5-bromo-2-(2-methyl-2-azabicyclo[2.2.2]octan-4-yl)-1,3-benzothiazole (2.37 g, crude).

LCMS(ESI): [M]⁺ m/z: calcd 337.2; found 338.2; Rt=1.026 min.

### Step 4: Synthesis of 2-(2-methyl-2-azabicyclo[2.2.2]octan-4-yl)-5-(4,4,5,5-tetramethyl-1,3,2-dioxaborolan-2-yl)benzo[d]thiazole

Prepared by general procedure scheme 4.1 step 2. Yield: 2.95 g of crude.

LCMS(ESI): [M]⁺ m/z: calcd 384.2; found 385.2; Rt=1.183 min.

### Step 5: Synthesis of (S)-tert-butyl 3-methyl-6-(2-(2-methyl-2-azabicyclo[2.2.2]octan-4-yl)benzo[d]thiazol-5-yl)-3,4-dihydropyridine-1(2H)-carboxylate

Prepared by general procedure scheme 4.1 step 3. Yield: 4.14 g of crude.

LCMS(ESI): [M]⁺ m/z: calcd 453.2; found 454.2; Rt=1.189 min.

### Step 6: Synthesis of (S)-5-(5-methyl-3,4,5,6-tetrahydropyridin-2-yl)-2-(2-methyl-2-azabicyclo[2.2.2]octan-4-yl)benzo[d]thiazole

Prepared by general procedure scheme 4.1 step 4. Yield: 1.98 g of crude.

LCMS(ESI): [M]⁺ m/z: calcd 353.2; found 354.2; Rt=0.723 min.

### Step 7: Synthesis of 2-(2-methyl-2-azabicyclo[2.2.2]octan-4-yl)-5-((2R,5S)-5-methylpiperidin-2-yl)benzo[d]thiazole

Prepared by general procedure scheme 4.1 step 5. Yield: 1.71 g (85.87%).

LCMS(ESI): [M]⁺ m/z: calcd 355.2; found 356.2; Rt=0.781 min.

### 3NNN. 2-(1-methyl-1,2,3,6-tetrahydropyridin-4-yl)-5-((2R,5S)-5-methylpiperidin-2-yl)benzo[d]thiazole

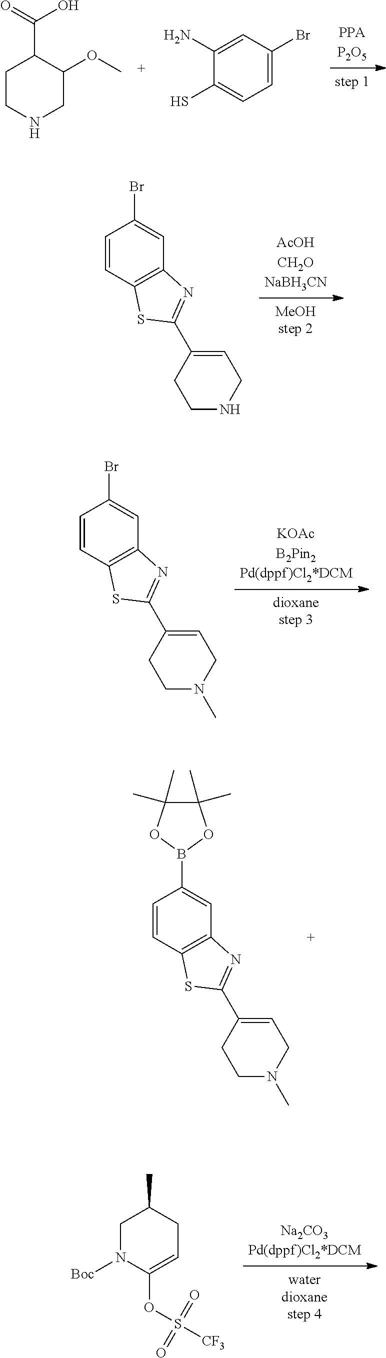

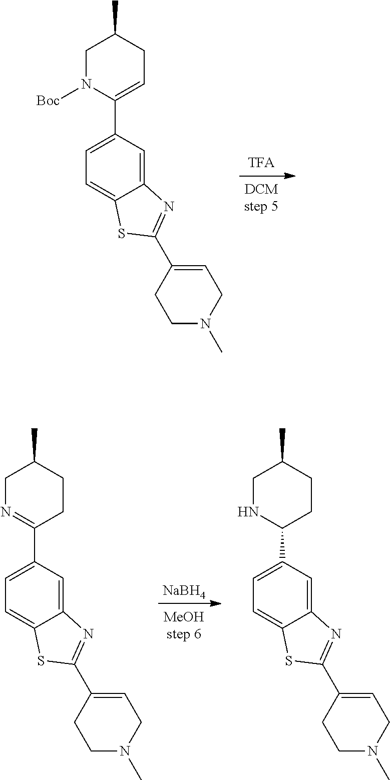 [SMILES: C[C@H]1CC=C(c2ccc3sc(C4=CCN(C)CC4)nc3c2)N(C(=O)OC(C)(C)C)C1 ; C[C@H]1CCC(c2ccc3sc(C4=CCN(C)CC4)nc3c2)=NC1 ; "NaBH4" ; C[C@H]1CC[C@H](c2ccc3sc(C4=CCN(C)CC4)nc3c2)NC1]

### Step 1: Synthesis of 5-bromo-2-(1,2,3,6-tetrahydropyridin-4-yl)benzo[d]thiazole

Prepared by general procedure scheme 4.1 step 1A. Yield: 2.5 g of crude.

LCMS(ESI): [M]⁺ m/z: calcd 295.2; found 296.2; Rt=0.961 min.

### Step 2: Synthesis of 5-bromo-2-(1-methyl-1,2,3,6-tetrahydropyridin-4-yl)benzo[d]thiazole

Formaldehyde, 37% w/w aq. soln., stab. with 7-8% MeOH (1.03 g, 12.70 mmol, 952.01 μL, 37% purity) and acetic acid (1.02 g, 16.94 mmol, 969.60 μL) were added to a stirred solution of 5-bromo-2-(1,2,3,6-tetrahydropyridin-4-yl)-1,3-benzothiazole (2.5 g, 8.47 mmol) in MeOH (100 mL) at 25° C. The resulting mixture was stirred at 25° C. for 1 hr, then sodium cyan borohydride (638.62 mg, 10.16 mmol) was added in one portion at 25° C. (foaming!). The reaction mixture was stirred at 25° C. for 18 hr, and then concentrated in vacuum. The residue was diluted with 10% aqueous sodium hydroxide solution (70 ml) and extracted with DCM (2*60 ml). The combined organic extracts were dried over sodium sulphate and concentrated in vacuum to afford crude 5-bromo-2-(1-methyl-3,6-dihydro-2H-pyridin-4-yl)-1,3-benzothiazole (2.5 g, 8.08 mmol, 95.46% yield) as light-brown gum, which was used directly in the next step.

LCMS(ESI): [M]⁺ m/z: calcd 309.2; found 310.2; Rt=2.242 min.

### Step 3: Synthesis of 2-(1-methyl-1,2,3,6-tetrahydropyridin-4-yl)-5-(4,4,5,5-tetramethyl-1,3,2-dioxaborolan-2-yl)benzo[d]thiazole

Prepared by general procedure scheme 4.1 step 2. Yield: 2.88 g of crude.

LCMS(ESI): [M]⁺ m/z: calcd 356.2; found 357.2; Rt=1.131 min.

### Step 4: Synthesis of (S)-tert-butyl 3-methyl-6-(2-(1-methyl-1,2,3,6-tetrahydropyridin-4-yl)benzo[d]thiazol-5-yl)-3,4-dihydropyridine-1(2H)-carboxylate

Prepared by general procedure scheme 4.1 step 3. Yield: 7 g of crude.

LCMS(ESI): [M]⁺ m/z: calcd 425.2; found 426.2; Rt=1.236 min.

### Step 5: Synthesis of (S)-2-(1-methyl-1,2,3,6-tetrahydropyridin-4-yl)-5-(5-methyl-3,4,5,6-tetrahydropyridin-2-yl)benzo[d]thiazole

TFA (29.60 g, 259.60 mmol, 20 mL) was added in one portion to a stirred solution of tert-butyl (3S)-3-methyl-6-[2-(1-methyl-3,6-dihydro-2H-pyridin-4-yl)-1,3-benzothiazol-5-yl]-3,4-dihydro-2H-pyridine-1-carboxylate (7 g, 16.45 mmol) in DCM (20 mL). The resulting solution was stirred at 25° C. for 0.5 hr, and then concentrated in vacuum. The residue was diluted with water (100 ml). The resulting solution of TFA salt of the product was decanted from dark-brown oily residue, which was additionally rinsed with water (2*25 ml). The combined aqueous solution was filtered through a cotton pad to remove traces of oily impurities, then basified to pH 11-12 with 10% aqueous sodium hydroxide solution and extracted with DCM (2*50 ml). The combined organic extracts were dried over sodium sulfate and concentrated in vacuum to afford 2-(1-methyl-3,6-dihydro-2H-pyridin-4-yl)-5-[(3S)-3-methyl-2,3,4,5-tetrahydropyridin-6-yl]-1,3-benzothiazole (2.7 g, 8.30 mmol, 50.44% yield) as brown solid, which was directly used in the next step.

LCMS(ESI): [M]⁺ m/z: calcd 325.2; found 326.2; Rt=0.673 min.

### Step 6: Synthesis of 2-(1-methyl-1,2,3,6-tetrahydropyridin-4-yl)-5-((2R,5S)-5-methylpiperidin-2-yl)benzo[d]thiazole

Prepared by general procedure scheme 4.1 step 5. Yield: 1.4 g of crude.

LCMS(ESI): [M]⁺ m/z: calcd 327.2; found 328.2; Rt=0.719 min.

### 3OOO. 2-((3aR,5s,6aS)-2-methyloctahydrocyclopenta[c]pyrrol-5-yl)-5-((2R,5S)-5-methylpiperidin-2-yl)benzo[d]thiazole

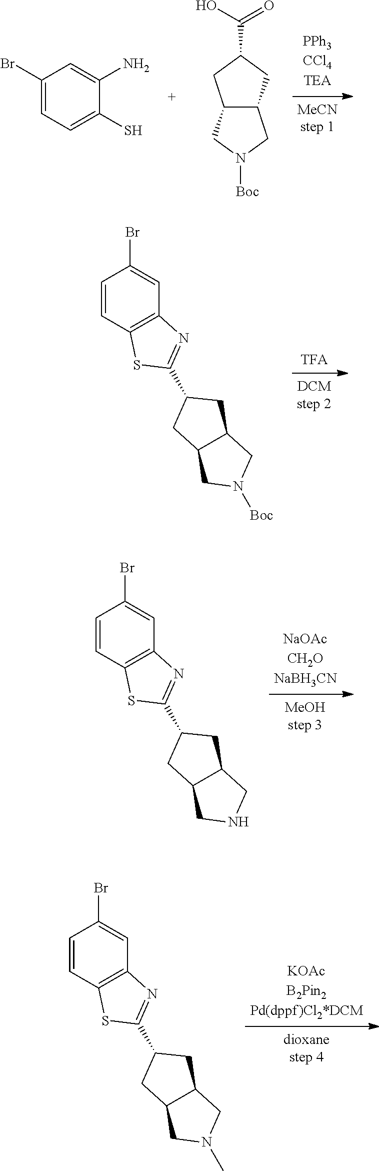 [SMILES: Nc1cc(Br)ccc1S ; CC(C)(C)OC(=O)N1C[C@H]2C[C@@H](C(=O)O)C[C@H]2C1 ; CC(C)(C)OC(=O)N1C[C@H]2C[C@H](c3nc4cc(Br)ccc4s3)C[C@H]2C1 ; Brc1ccc2sc([C@@H]3C[C@H]4CNC[C@H]4C3)nc2c1 ; CN1C[C@H]2C[C@H](c3nc4cc(Br)ccc4s3)C[C@H]2C1]

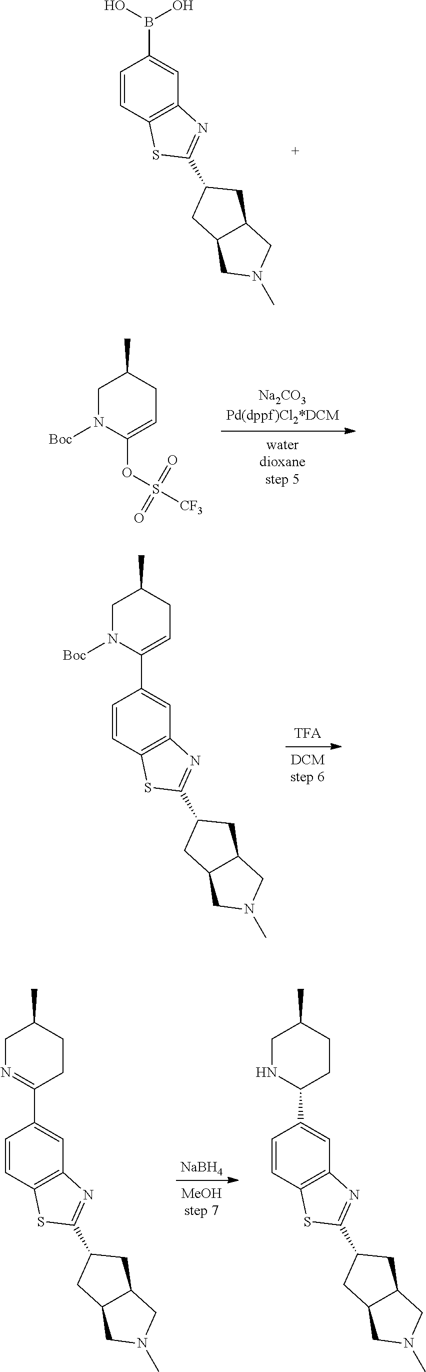

### Step 1: Synthesis of (3aR,5s,6aS)-tert-butyl 5-(5-bromobenzo[d]thiazol-2-yl)hexahydrocyclopenta[c]pyrrole-2(1H)-carboxylate

Triphenylphosphine (6.17 g, 23.52 mmol) was added in one portion to the solution of (3aR,6aS)-2-tert-butoxycarbonyl-3,3a,4,5,6,6a-hexahydro-1H-cyclopenta[c]pyrrole-5-carboxylic acid (2.50 g, 9.80 mmol), 2-amino-4-bromo-benzenethiol (2 g, 9.80 mmol), carbon tetrachloride (8.74 g, 56.84 mmol) and TEA (4.96 g, 49.00 mmol, 6.83 mL). Resulting reaction mixture was briefly warmed up to approximately 50-60° C. due to exothermic reaction. After that, it was stirred at 20° C. for 18 hr. Then, volatiles were removed under reduced pressure and residue was triturated with MTBE (100 ml). Resulting light precipitate was filtered off Filtrate was concentrated under reduced pressure and residue was purified by gradient column chromatography (SiO₂, Hexane/MTBE), affording tert-butyl 5-(5-bromo-1,3-benzothiazol-2-yl)-3,3a,4,5,6,6a-hexahydro-1H-cyclopenta[c]pyrrole-2-carboxylate (1.55 g, 3.66 mmol, 37.36% yield).

LCMS(ESI): [M-t-Bu]⁺ m/z: calcd 367.2; found 368.2; Rt=1.737 min.

### Step 2: Synthesis of 5-bromo-2-((3aR,5s,6aS)-octahydrocyclopenta[c]pyrrol-5-yl)benzo[d]thiazole

TFA (4.17 g, 36.61 mmol, 2.82 mL) was added to the solution of tert-butyl 5-(5-bromo-1,3-benzothiazol-2-yl)-3,3a,4,5,6,6a-hexahydro-1H-cyclopenta[c]pyrrole-2-carboxylate (1.55 g, 3.66 mmol) in DCM (7.18 mL). Resulting mixture was stirred at 20° C. for 5 hr. Then, it was concentrated under reduced pressure, leaving 5-bromo-2-[(3aR,6aS)-1,2,3,3a,4,5,6,6a-octahydrocyclopenta[c]pyrrol-5-yl]-1,3-benzothiazole (1.2 g, 2.75 mmol, 75.13% yield, TFA).

LCMS(ESI): [M]⁺ m/z: calcd 324.2; found 325.2; Rt=0.999 min.

### Step 3: Synthesis of 5-bromo-2-((3aR,5s,6aS)-2-methyloctahydrocyclopenta[c]pyrrol-5-yl)benzo[d]thiazole

Formaldehyde, 37% w/w aq. soln., stab. with 7-8% MeOH (222.93 mg, 7.42 mmol, 205.85 μL) and sodium acetate (609.07 mg, 7.42 mmol, 398.60 μL) were added to the solution of 5-bromo-2-[(3aR,6aS)-1,2,3,3a,4,5,6,6a-octahydrocyclopenta[c]pyrrol-5-yl]-1,3-benzothiazole (1.2 g, 3.71 mmol) in MeOH (19.40 mL). Resulting mixture was stirred at 20° C. for 1 hr before sodium cyan borohydride (466.58 mg, 7.42 mmol) was added thereto. After that, stirring was continued for 16 hr. Then, solvent was removed under reduced pressure and residue was partitioned between 15% aq. K₂CO₃ solution (30 ml) and DCM (50 ml). Organic layer was separated, dried over solid K₂CO₃ and concentrated under reduced pressure, leaving 5-bromo-2-[(3aR,6aS)-2-methyl-3,3a,4,5,6,6a-hexahydro-1H-cyclopenta[c]pyrrol-5-yl]-1,3-benzothiazole (1.2 g, 3.56 mmol, 95.84% yield).

LCMS(ESI): [M]⁺ m/z: calcd 337.2; found 338.2; Rt=0.918 min.

### Step 4: Synthesis of (2-((3aR,5s,6aS)-2-methyloctahydrocyclopenta[c]pyrrol-5-yl)benzo[d]thiazol-5-yl)boronic acid

Prepared by general procedure scheme 4.1 step 2. Yield: 0.5 g of crude.

LCMS(ESI): [M]⁺ m/z: calcd 302.2; found 303.2; Rt=0.618 min.

### Step 5: Synthesis of (S)-tert-butyl 3-methyl-6-(2-((3aR,5s,6aS)-2-methyloctahydrocyclopenta[c]pyrrol-5-yl)benzo[d]thiazol-5-yl)-3,4-dihydropyridine-1(2H)-carboxylate

Prepared by general procedure scheme 4.1 step 3. Yield: 0.7 g of crude.

LCMS(ESI): [M]⁺ m/z: calcd 453.2; found 454.2; Rt=1.152 min.

### Step 6: Synthesis of 5-((S)-5-methyl-3,4,5,6-tetrahydropyridin-2-yl)-2-((3aR,5s,6aS)-2-methyloctahydrocyclopenta[c]pyrrol-5-yl)benzo[d]thiazole

Prepared by general procedure scheme 4.1 step 4. Yield: 0.5 g of crude.

LCMS(ESI): [M]⁺ m/z: calcd 353.2; found 354.2; Rt=0.603 min.

### Step 7: Synthesis of 2-((3aR,5s,6aS)-2-methyloctahydrocyclopenta[c]pyrrol-5-yl)-5-((2R,5S)-5-methylpiperidin-2-yl)benzo[d]thiazole

Prepared by general procedure scheme 4.1 step 5. Yield: 0.3 g of crude.

LCMS(ESI): [M]⁺ m/z: calcd 355.2; found 356.2; Rt=0.710 min.

### 3PPP. N,N-dimethyl-2-(5-((2R,5S)-5-methylpiperidin-2-yl)benzo[d]thiazol-2-yl)propan-1-amine

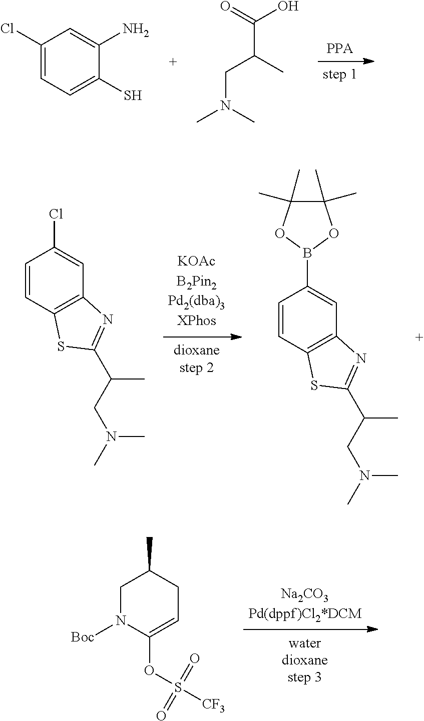

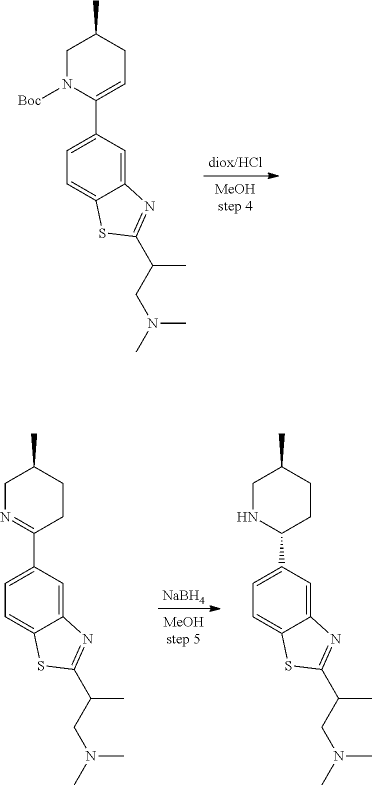 [SMILES: CC(CN(C)C)c1nc2cc(C3=CC[C@H](C)CN3C(=O)OC(C)(C)C)ccc2s1 ; CC(CN(C)C)c1nc2cc(C3=NC[C@@H](C)CC3)ccc2s1 ; "NaBH4" ; CC(CN(C)C)c1nc2cc([C@H]3CC[C@H](C)CN3)ccc2s1]

### Step 1: Synthesis of 2-(5-chlorobenzo[d]thiazol-2-yl)-N,N-dimethylpropan-1-amine

The stirred solution of 2-amino-4-chloro-benzenethiol (1.5 g, 9.40 mmol) and 2-amino-4-chloro-benzenethiol (1.5 g, 9.40 mmol) in PPA (10 mL) was allowed to stir at 140° C. for 16 hr. Upon completion, the reaction mixture was quenched with water (200 mL) and neutralized by NaOH to pH=8. The aqueous phase was extracted with EtOAc (2*20 mL). The combined organic phase was dried over Na₂SO₄ and concentrated under reduced pressure. The desired product 2-(5-chloro-1,3-benzothiazol-2-yl)-N,N-dimethyl-propan-1-amine (1.5 g, 5.89 mmol, 62.66% yield) was isolated.

LCMS(ESI): [M]⁺ m/z: calcd 254.2; found 255.2; Rt=0.672 min.

### Step 2: Synthesis of N,N-dimethyl-2-(5-(4,4,5,5-tetramethyl-1,3,2-dioxaborolan-2-yl)benzo[d]thiazol-2-yl)propan-1-amine

To a stirred solution of 2-(5-chloro-1,3-benzothiazol-2-yl)-N,N-dimethyl-propan-1-amine (1.5 g, 5.89 mmol) and 4,4,5,5-tetramethyl-2-(4,4,5,5-tetramethyl-1,3,2-dioxaborolan-2-yl)-1,3,2-dioxaborolane (1.50 g, 5.89 mmol) in dioxane (30 mL) were added Pd₂(dba)₃ (1.08 g, 1.18 mmol) and XPhos (1.12 g, 2.35 mmol). The resulting suspension was degassed with argon at 50° C. for 0.5 hr. Potassium acetate (1.2 g, 12.23 mmol, 764.33 μL) was added. The reaction mixture was stirred at 100° C. for 16 hr. Upon completion, the reaction mixture was concentrated under reduced pressure, quenched with water (50 mL), the aqueous phase was extracted with CHCl₃ (2*50 mL). The organic phase was extracted with 10% HCl (2*50 ml). The aqueous phase was neutralized by NaHCO₃ to pH=8, extracted with CHCl₃ (2*50 mL). The organic phase was dried over Na₂SO₄ and evaporated in vacuum. The desired product N,N-dimethyl-2-[5-(4,4,5,5-tetramethyl-1,3,2-dioxaborolan-2-yl)-1,3-benzothiazol-2-yl]propan-1-amine (1.4 g, 4.04 mmol, 68.67% yield) was isolated.

LCMS(ESI): [M]⁺ m/z: calcd 346.2; found 347.2; Rt=1.121 min.

### Step 3: Synthesis of (3S)-tert-butyl 6-(2-(1-(dimethylamino)propan-2-yl)benzo[d]thiazol-5-yl)-3-methyl-3,4-dihydropyridine-1(2H)-carboxylate

Prepared by general procedure scheme 4.1 step 3. Yield: 0.7 g of crude.

LCMS(ESI): [M]⁺ m/z: calcd 415.2; found 416.2; Rt=1.238 min.

### Step 4: Synthesis of N,N-dimethyl-2-(5-((S)-5-methyl-3,4,5,6-tetrahydropyridin-2-yl)benzo[d]thiazol-2-yl)propan-1-amine

The stirred solution of tert-butyl (3S)-6-[2-[2-(dimethylamino)-1-methyl-ethyl]-1,3-benzothiazol-5-yl]-3-methyl-3,4-dihydro-2H-pyridine-1-carboxylate (700.00 mg, 1.68 mmol) in MeOH (10 mL) and diox/HCl (10 mL) was allowed to stir at 25° C. for 16 hr. Upon completion, the reaction mixture was evaporated, the crude product was quenched with water (20 mL) and neutralized by NaHCO₃ to pH=8. The aqueous phase was extracted with CHCl₃ (2*20 mL). The combined organic phase was dried over Na₂SO₄ and concentrated under reduced pressure. The desired product N,N-dimethyl-2-[5-[(3S)-3-methyl-2,3,4,5-tetrahydropyridin-6-yl]-1,3-benzothiazol-2-yl]propan-1-amine (0.5 g, 1.58 mmol, 94.10% yield) was isolated.

LCMS(ESI): [M]⁺ m/z: calcd 315.2; found 316.2; Rt=0.666 min.

### Step 5: Synthesis of N,N-dimethyl-2-(5-((2R,5S)-5-methylpiperidin-2-yl)benzo[d]thiazol-2-yl)propan-1-amine

Prepared by general procedure scheme 4.1 step 5. Yield: 0.5 g of crude.

LCMS(ESI): [M]⁺ m/z: calcd 317.2; found 318.2; Rt=0.471 min.

### 3QQQ. Synthesis of 2-(1-azabicyclo[2.2.1]heptan-3-yl)-5-((2R,5S)-5-methylpiperidin-2-yl)benzo[d]thiazole (c)

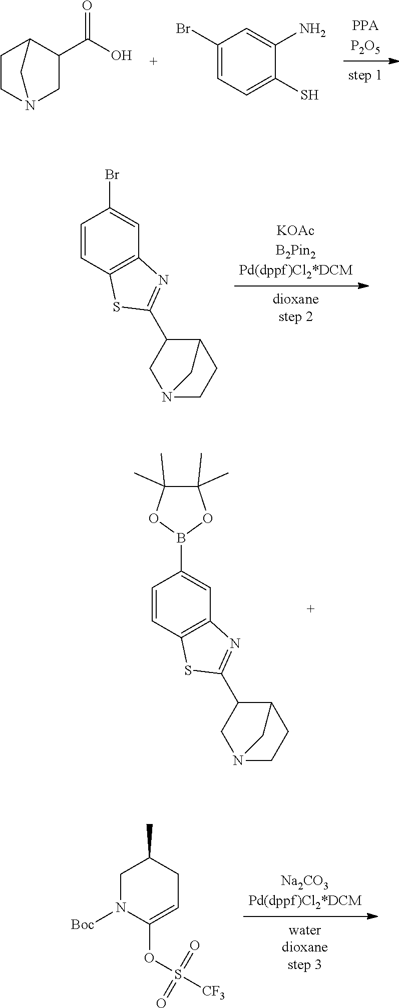 [SMILES: O=C(O)C1CN2CCC1C2 ; Nc1cc(Br)ccc1S ; Brc1ccc2sc(C3CN4CCC3C4)nc2c1 ; CC1(C)OB(c2ccc3sc(C4CN5CCC4C5)nc3c2)OC1(C)C ; C[C@H]1CC=C(OS(=O)(=O)C(F)(F)F)N(C(=O)OC(C)(C)C)C1]

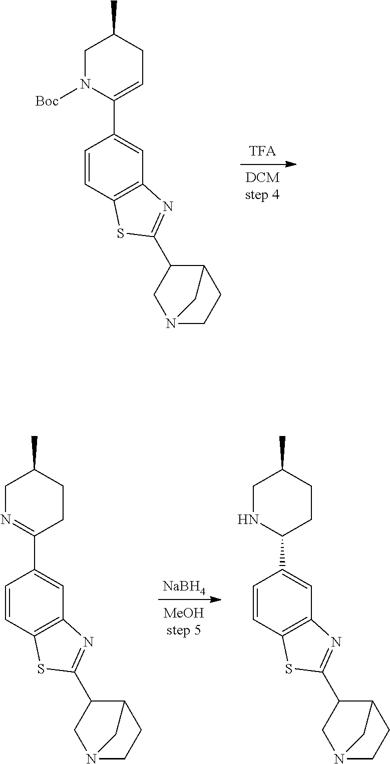 [SMILES: C[C@H]1CC=C(c2ccc3sc(C4CN5CCC4C5)nc3c2)N(C(=O)OC(C)(C)C)C1 ; C[C@H]1CCC(c2ccc3sc(C4CN5CCC4C5)nc3c2)=NC1 ; "NaBH4" ; C[C@H]1CC[C@H](c2ccc3sc(C4CN5CCC4C5)nc3c2)NC1]

### Step 1: Synthesis of 2-(1-azabicyclo[2.2.1]heptan-3-yl)-5-bromobenzo[d]thiazole

Prepared by general procedure scheme 4.1 step 1A. Yield: 1.4 g of crude.

LCMS(ESI): [M]⁺ m/z: calcd 308.2; found 309.2; Rt=0.915 min.

### Step 2: Synthesis of 2-(1-azabicyclo[2.2.1]heptan-3-yl)-5-(4,4,5,5-tetramethyl-1,3,2-dioxaborolan-2-yl)benzo[d]thiazole

Prepared by general procedure scheme 4.1 step 2. Yield: 1.8 g of crude.

LCMS(ESI): [M]⁺ m/z: calcd 356.2; found 357.2; Rt=1.138 min.

### Step 3: Synthesis of tert-butyl (3S)-6-(2-(1-azabicyclo[2.2.1]heptan-3-yl)benzo[d]thiazol-5-yl)-3-methyl-3,4-dihydropyridine-1(2H)-carboxylate

Prepared by general procedure scheme 4.1 step 3. Yield: 1.4 g of crude.

LCMS(ESI): [M]⁺ m/z: calcd 425.2; found 426.2; Rt=0.920 min.

### Step 4: Synthesis of 2-(1-azabicyclo[2.2.1]heptan-3-yl)-5-((S)-5-methyl-3,4,5,6-tetrahydropyridin-2-yl)benzo[d]thiazole

Prepared by general procedure scheme 4.1 step 4. Yield: 0.6 g of crude.

LCMS(ESI): [M]⁺ m/z: calcd 325.2; found 326.2; Rt=0.534 min.

### Step 5: Synthesis of 2-(1-azabicyclo[2.2.1]heptan-3-yl)-5-((2R,5S)-5-methylpiperidin-2-yl)benzo[d]thiazole (c)

Prepared by general procedure scheme 4.1 step 5. Yield: 0.6 g of crude.

LCMS(ESI): [M]⁺ m/z: calcd 327.2; found 328.2; Rt=0.700 min.

### 3RRR. 2-(1-methyl-4-piperidyl)-7-[(2R,5S)-5-methyl-2-piperidyl]quinoline

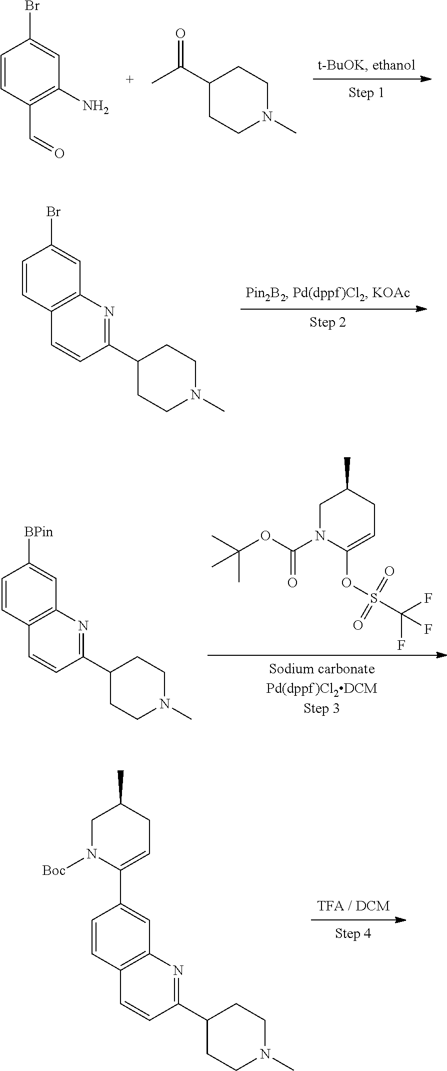

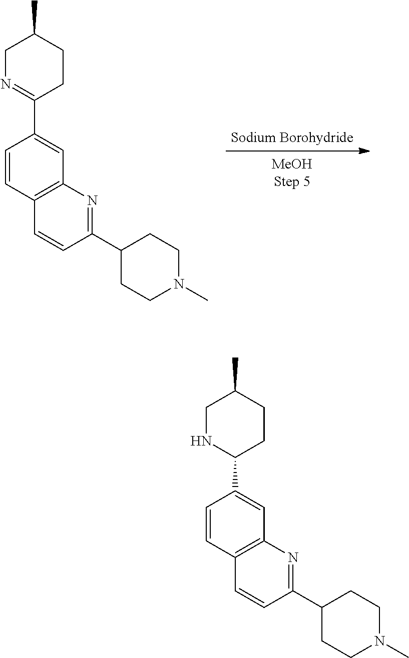 [SMILES: C[C@H]1CCC(c2ccc3ccc(C4CCN(C)CC4)nc3c2)=NC1 ; "Sodium Borohydride" ; C[C@H]1CC[C@H](c2ccc3ccc(C4CCN(C)CC4)nc3c2)NC1]

### Step 1: 7-bromo-2-(1-methyl-4-piperidyl)quinoline

2-amino-4-bromo-benzaldehyde (1.3 g, 6.50 mmol), 1-(1-methyl-4-piperidyl)ethanone (917.72 mg, 6.50 mmol) and Sodium tert-butoxide (1.25 g, 13.00 mmol) were mixed in ethanol (20 mL) and stirred for 12 hr at 80° C. The RM was concentrated in vacuo, then treated with DCM, washed with water. Organic phase was dried over Na₂SO₄ and evaporated to give 7-bromo-2-(1-methyl-4-piperidyl)quinoline (1.65 g, crude) LCMS(ESI): [M+1]⁺ m/z: calcd 304.1; found 305; Rt=0.934 min.

### Step 2: 2-(1-methyl-4-piperidyl)-7-(4,4,5,5-tetramethyl-1,3,2-dioxaborolan-2-yl)quinoline

A mixture of 7-bromo-2-(1-methyl-4-piperidyl)quinoline (1.95 g, 6.39 mmol) (300 mg), Bis(pinacolato) diboron (2.11 g, 8.31 mmol) and Potassium Acetate (1.88 g, 19.17 mmol, 1.20 mL) in Dioxane (30.03 mL) was degassed with argon for 10 min. Pd(dppf)Cl₂*DCM (521.75 mg, 638.90 mol) was next added and the reaction mixture was heated at 80° C. for 12 hr. The reaction mixture was filtered, then concentrated in vacuo. The residue was treated with mixture of MTBE-Hex (1:1), filtered and then concentrated under reduced pressure to give 2-(1-methyl-4-piperidyl)-7-(4,4,5,5-tetramethyl-1,3,2-dioxaborolan-2-yl)quinoline (3 g, crude).

LCMS(ESI): [M+1]⁺ m/z: calcd 352.3; found 353.2; Rt=1.119 min.

### Step 3: tert-butyl (3S)-3-methyl-6-[2-(1-methyl-4-piperidyl)-7-quinolyl]-3,4-dihydro-2H-pyridine-1-carboxylate

2-(1-methyl-4-piperidyl)-7-(4,4,5,5-tetramethyl-1,3,2-dioxaborolan-2-yl)quinoline (2.8 g, 7.95 mmol), Sodium carbonate (2.53 g, 23.84 mmol, 998.15 μL), tert-butyl (3S)-3-methyl-6-(trifluoromethylsulfonyloxy)-3,4-dihydro-2H-pyridine-1-carboxylate (2.74 g, 7.95 mmol) and Pd(dppf)Cl2*DCM (0.5 g, 612.75 mol) were mixed in H₂O (15 mL) and dioxane (50 mL) under argon and stirred at 75° C. for 12 hr. Reaction mixture was diluted with water and desired product extracted with DCM, dried over Na₂SO₄ and concentrated in vacuo. tert-butyl (3S)-3-methyl-6-[2-(1-methyl-4-piperidyl)-7-quinolyl]-3,4-dihydro-2H-pyridine-1-carboxylate (4 g, crude) was obtained.

LCMS(ESI): [M+1]⁺ m/z: calcd 421.3; found 422.2; Rt=1.174 min.

### Step 4: 2-(1-methyl-4-piperidyl)-7-[(3S)-3-methyl-2,3,4,5-tetrahydropyridin-6-yl]quinoline

tert-butyl (3S)-3-methyl-6-[2-(1-methyl-4-piperidyl)-7-quinolyl]-3,4-dihydro-2H-pyridine-1-carboxylate (4 g, 9.49 mmol) was dissolved in DCM (40 mL) and CF₃COOH (15 g, 9.49 mmol) was added. The RM was stirred for 2 hr, then concentrated. The residue was treated with MTBE two times. Black gum was treated with aq. solution of NaHCO₃ and then extracted with DCM. Organic phase was dried over Na₂SO₄ and then concentrated in vacuo to give 2-(1-methyl-4-piperidyl)-7-[(3S)-3-methyl-2,3,4,5-tetrahydropyridin-6-yl]quinoline (1 g, crude)

LCMS(ESI): [M+1]⁺ m/z: calcd 321.2; found 322.2; Rt=0.682 min.

### Step 5: 2-(1-methyl-4-piperidyl)-7-[(2R,5S)-5-methyl-2-piperidyl]quinoline

To a solution of 2-(1-methyl-4-piperidyl)-7-[(3S)-3-methyl-2,3,4,5-tetrahydropyridin-6-yl]quinoline (1 g, 2.49 mmol) in methanol (30 mL) Sodium Borohydride (188.30 mg, 4.98 mmol, 175.33 μL) was added, then stirred overnight. The RM was concentrated in vacuo, then treated with DCM, filtered and evaporated to give 2-(1-methyl-4-piperidyl)-7-[(2R,5S)-5-methyl-2-piperidyl]quinoline (0.75 g, crude)

LCMS(ESI): [M+1]⁺ m/z: calcd 323.2; found 324.2; Rt=0.705 min.

### 3SSS. 2-((1,4-dimethylpiperidin-4-yl)methyl)-5-((2R,5S)-5-methylpiperidin-2-yl)benzo[d]thiazole

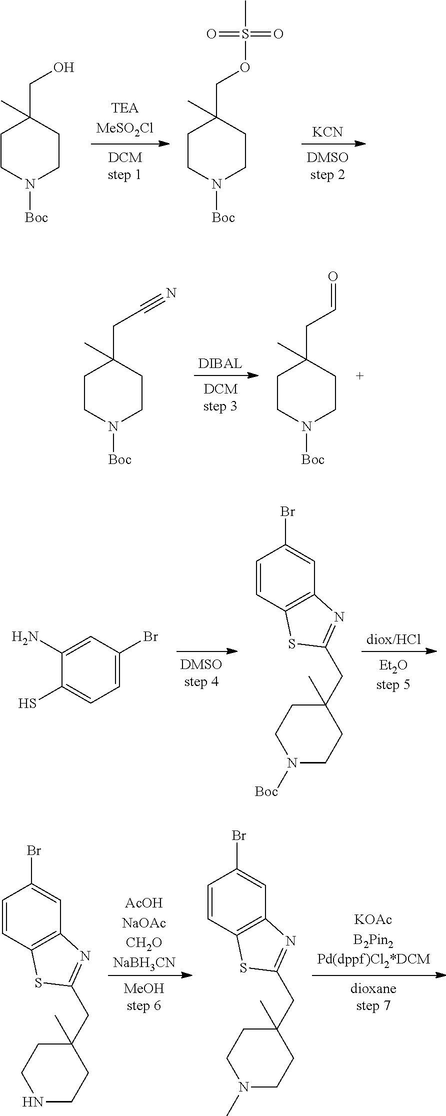

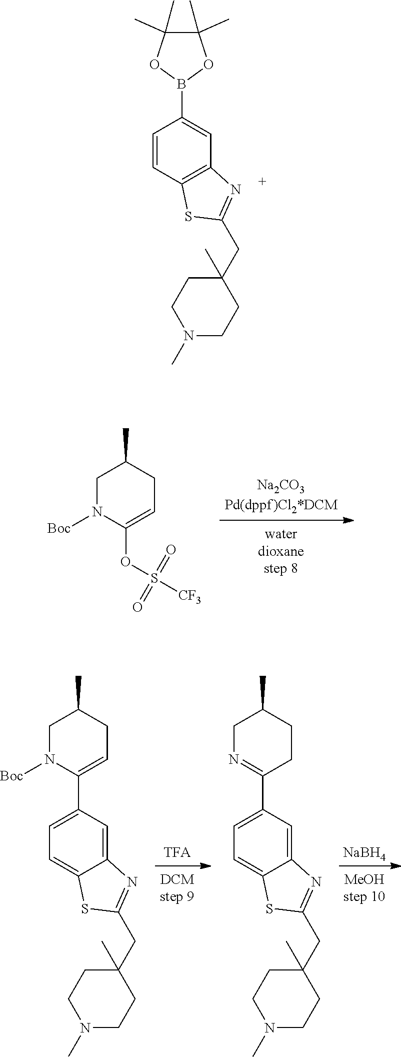 [SMILES: CN1CCC(C)(Cc2nc3cc(B4OC(C)(C)C(C)(C)O4)ccc3s2)CC1 ; C[C@H]1CC=C(OS(=O)(=O)C(F)(F)F)N(C(=O)OC(C)(C)C)C1 ; C[C@H]1CC=C(c2ccc3sc(CC4(C)CCN(C)CC4)nc3c2)N(C(=O)OC(C)(C)C)C1 ; C[C@H]1CCC(c2ccc3sc(CC4(C)CCN(C)CC4)nc3c2)=NC1 ; "NaBH4"]

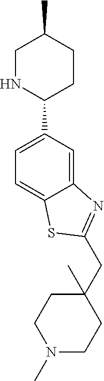 [SMILES: C[C@H]1CC[C@H](c2ccc3sc(CC4(C)CCN(C)CC4)nc3c2)NC1]

### Step 1: Synthesis of tert-butyl 4-methyl-4-(((methylsulfonyl)oxy)methyl)piperidine-1-carboxylate

To a solution of tert-butyl 4-(hydroxymethyl)-4-methyl-piperidine-1-carboxylate (8.97 g, 35.20 mmol), TEA (4.27 g, 42.25 mmol, 5.89 mL) in DCM (17.21 mL) was added methansulfonyl chloride (4.44 g, 38.73 mmol, 3.00 mL) portion wise at 0° C. and allowed to warm to rt. The resulting solution was washed with 10% aq. HCl and brine, dried over Na₂SO₄ and evaporated to dryness to give tert-butyl 4-methyl-4-(methylsulfonyloxymethyl)piperidine-1-carboxylate (9 g, 29.28 mmol, 83.16% yield) as a brownish gum.

¹H NMR (500 MHz, CDCl₃) δ (ppm) 1.05 (s, 3H), 1.34 (m, 2H), 1.43 (s, 9H), 1.48 (m, 2H), 2.99 (s, 3H), 3.14 (m, 2H), 3.66 (m, 2H), 3.94 (s, 2H).

### Step 2: Synthesis of tert-butyl 4-(cyanomethyl)-4-methylpiperidine-1-carboxylate

To a solution of tert-butyl 4-methyl-4-(methylsulfonyloxymethyl)piperidine-1-carboxylate (10 g, 32.53 mmol) in DMSO (75 mL) was added potassium cyanide (8.47 g, 130.12 mmol) and the resulting mixture was heated to 130° C. for 12 hr. The resulting mixture was poured into water, extracted with EtOAc (3×100 mL), combined organics were washed with water, brine, dried and evaporated to give tert-butyl 4-(cyanomethyl)-4-methyl-piperidine-1-carboxylate (7.3 g, crude) as a yellow gum.

¹H NMR (500 MHz, CDCl₃) δ (ppm) 1.14 (s, 3H), 1.44 (s, 9H), 1.46 (m, 4H), 2.29 (s, 2H), 3.22 (m, 2H), 3.57 (m, 2H).

### Step 3: Synthesis of tert-butyl 4-methyl-4-(2-oxoethyl)piperidine-1-carboxylate

To a solution of tert-butyl 4-(cyanomethyl)-4-methyl-piperidine-1-carboxylate (6.93 g, 29.08 mmol) in DCM (250 mL) at −30° C. was added DIBAL (10.34 g, 72.69 mmol, 72.69 mL) slowly, and the mixture was stirred at the same temperature for 30 min. 15 mL of Methanol was added followed by 25 mL of the saturated citric acid solution, and the reaction mixture was stirred allowed at rt for 15 min. The reaction mixture was filtered through a pad of celite, and the filtrate was diluted with 250 mL of DCM. The organic layer was washed with brine solution and concentrated in vacuum to give tert-butyl 4-methyl-4-(2-oxoethyl)piperidine-1-carboxylate (4.2 g, crude) as a light yellow oil.

¹H NMR (500 MHz, DMSO-d₆) δ (ppm) 1.13 (s, 3H), 1.40 (s, 9H), 1.52 (m, 4H), 2.34 (s, 2H), 3.29 (m, 2H), 3.51 (m, 2H), 9.84 (s, 1H).

### Step 4: Synthesis of tert-butyl 4-((5-bromobenzo[d]thiazol-2-yl)methyl)-4-methylpiperidine-1-carboxylate

Prepared by general procedure scheme 4.1 step 1B. Yield: 4.8 g (81.05%).

¹H NMR (500 MHz, CDCl₃) δ (ppm) 1.08 (s, 3H), 1.43 (s, 9H), 1.46 (m, 2H), 1.57 (m, 2H), 3.04 (s, 2H), 3.23 (m, 2H), 3.66 (m, 2H), 7.44 (d, 1H), 7.67 (d, 1H), 8.12 (s, 1H).

### Step 5: Synthesis of 5-bromo-2-((4-methylpiperidin-4-yl)methyl)benzo[d]thiazole

To a solution of tert-butyl 4-[(5-bromo-1,3-benzothiazol-2-yl)methyl]-4-methyl-piperidine-1-carboxylate (5.4 g, 12.69 mmol) in Et₂O (20.00 mL) was added hydrogen chloride solution 4.0M in dioxane (24.00 g, 658.24 mmol, 30.00 mL) at 21° C. The resulting mixture was left to stir for 6 hr. The resulting mixture was evaporated to dryness and was used in the next step without further purification. 5-Bromo-2-[(4-methyl-4-piperidyl)methyl]-1,3-benzothiazole (4.5 g, crude, 2HCl) was obtained as a beige solid.

LCMS(ESI): [M]⁺ m/z: calcd 325.2; found 326.2; Rt=2.325 min.

### Step 6: Synthesis of 5-bromo-2-((1,4-dimethylpiperidin-4-yl)methyl)benzo[d]thiazole

Formaldehyde, 37% w/w aq. soln., stab. with 7-8% MeOH (1.01 g, 12.43 mmol, 931.63 μL, 37% purity) and acetic acid (2.04 g, 33.90 mmol, 1.94 mL) were added to the solution of 5-bromo-2-[(4-methyl-4-piperidyl)methyl]-1,3-benzothiazole (4.5 g, 11.30 mmol, 2HCl) and sodium acetate, anhydrous (1.85 g, 22.60 mmol, 1.21 mL) in MeOH. Resulting mixture was stirred at 21° C. for 1 hr before sodium cyan borohydride (1.42 g, 22.60 mmol) was added thereto. After that, stirring was continued for 8 hr. Then, the solvent was removed under reduced pressure, and residue was partitioned between 10% aq. NaOH solution (20 ml) and DCM (40 ml). The organic layer was separated, evaporated. 5-bromo-2-[(1,4-dimethyl-4-piperidyl)methyl]-1,3-benzothiazole (4.3 g, crude) was obtained as a yellow gum.

LCMS(ESI): [M]⁺ m/z: calcd 339.2; found 340.2; Rt=2.474 min.

### Step 7: Synthesis of 2-((1,4-dimethylpiperidin-4-yl)methyl)-5-(4,4,5,5-tetramethyl-1,3,2-dioxaborolan-2-yl)benzo[d]thiazole

Prepared by general procedure scheme 4.1 step 2. Yield: 4.5 g of crude.

LCMS(ESI): [M]⁺ m/z: calcd 386.2; found 387.2; Rt=2.490 min.

### Step 8: Synthesis of (S)-tert-butyl 6-(2-((1,4-dimethylpiperidin-4-yl)methyl)benzo[d]thiazol-5-yl)-3-methyl-3,4-dihydropyridine-1(2H)-carboxylate

Prepared by general procedure scheme 4.1 step 3. Yield: 7.5 g of crude.

LCMS(ESI): [M]⁺ m/z: calcd 455.2; found 456.2; Rt=3.572 min.

### Step 9: Synthesis of (S)-2-((1,4-dimethylpiperidin-4-yl)methyl)-5-(5-methyl-3,4,5,6-tetrahydropyridin-2-yl)benzo[d]thiazole

To a solution of tert-butyl (3S)-6-[2-[(1,4-dimethyl-4-piperidyl)methyl]-1,3-benzothiazol-5-yl]-3-methyl-3,4-dihydro-2H-pyridine-1-carboxylate (7.5 g, 16.46 mmol) in DCM (18.66 mL) was added TFA (9.38 g, 82.30 mmol, 6.34 mL) in one portion and the resulting mixture was left to stir overnight at rt. The reaction mixture was quenched with aq NaOH (30%). Water was extracted with DCM (3×50 mL). Combined organics were dried over Na₂SO₄ and evaporated to give a residue which was used in the next step without further purification.

LCMS(ESI): [M]⁺ m/z: calcd 355.2; found 356.2; Rt=1.424 min.

### Step 10: Synthesis of 2-((1,4-dimethylpiperidin-4-yl)methyl)-5-((2R,5S)-5-methylpiperidin-2-yl)benzo[d]thiazole

Prepared by general procedure scheme 4.1 step 5. Yield: 0.77 g (15.31%).

LCMS(ESI): [M]⁺ m/z: calcd 357.2; found 358.2; Rt=0.665 min.

### 3TTT. S)—N,N-dimethyl-1-(3-((2R,5S)-5-methylpiperidin-2-yl)phenoxy)propan-2-amine

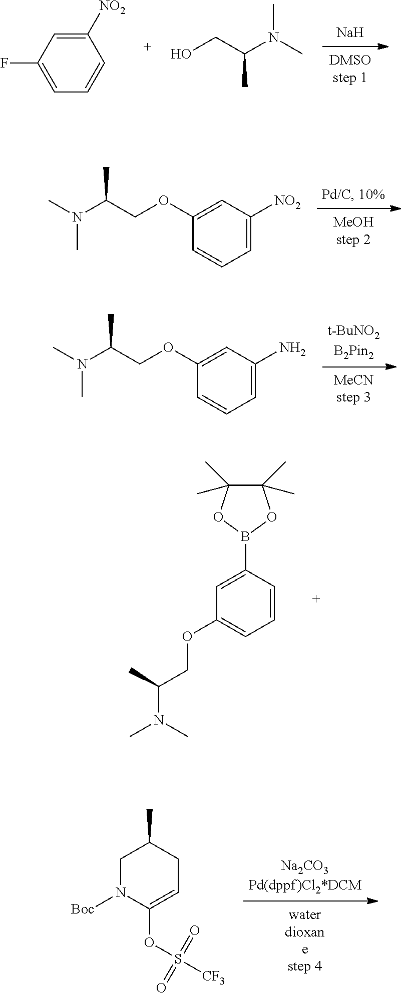

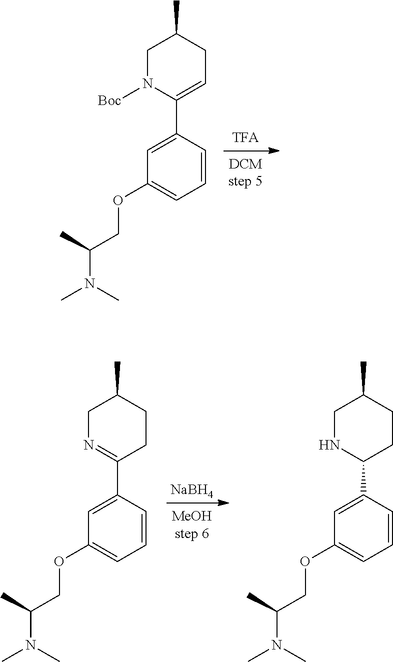 [SMILES: C[C@H]1CC=C(c2cccc(OC[C@H](C)N(C)C)c2)N(C(=O)OC(C)(C)C)C1 ; C[C@H]1CCC(c2cccc(OC[C@H](C)N(C)C)c2)=NC1 ; "NaBH4" ; C[C@H]1CC[C@H](c2cccc(OC[C@H](C)N(C)C)c2)NC1]

### Step 1: Synthesis of (S)—N,N-dimethyl-1-(3-nitrophenoxy)propan-2-amine

Sodium hydride (in oil dispersion) 60% dispersion in mineral oil (405.15 mg, 10.13 mmol, 60% purity) was added to DMSO (7 mL) and the resulting mixture was stirred for 30 min. A solution of (2S)-2-(dimethylamino)propan-1-ol (950 mg, 9.21 mmol) in DMSO (2.5 mL) was added dropwise to the previous mixture and the resulting mixture was stirred for 30 min. 1-Fluoro-3-nitro-benzene (1.30 g, 9.21 mmol, 980.65 μL) was added dropwise to the previous mixture and the resulting mixture was stirred for 1.5 hr. The reaction mixture was quenched with aq·NH₄C₁ (50 ml) and the resulting mixture was extracted with EtOAc (3*50 ml). Combined organic layers were washed with brine (3*40 ml), dried over Na₂SO₄, filtered and concentrated in vacuum. The residue was purified by flash chromatography (gradient MeOH in MTBE from 0% to 50%) to obtain (2S)—N,N-dimethyl-1-(3-nitrophenoxy)propan-2-amine (1.14 g, 5.07 mmol, 55.11% yield).

LCMS(ESI): [M]⁺ m/z: calcd 224.2; found 225.2; Rt=0.758 min.

### Step 2: Synthesis of (S)-3-(2-(dimethylamino)propoxy)aniline

(2S)—N,N-Dimethyl-1-(3-nitrophenoxy)propan-2-amine (1.14 g, 5.07 mmol) was dissolved in MeOH (20 mL) and palladium, 10% on carbon, Type 487, dry (270.02 mg, 2.54 mmol) was added thereto. The resulting mixture was evacuated and backfilled three times with hydrogen. The reaction mixture was hydrogenated at 1 atm (balloon) overnight. 25% of starting material has left, according to LCMS of aliquot. 200 mg of 10% Pd/C was added to the reaction mixture and the resulting mixture was evacuated and backfilled three times with hydrogen. The reaction mixture was hydrogenated at 1 atm (balloon) overnight. The catalyst was filtered off and the filtrate was concentrated in vacuum to obtain 3-[(2S)-2-(dimethylamino)propoxy]aniline (948 mg, 4.88 mmol, 96.16% yield).

LCMS(ESI): [M]⁺ m/z: calcd 194.2; found 195.2; Rt=0.203 min.

### Step 3: Synthesis of (S)—N,N-dimethyl-1-(3-(4,4,5,5-tetramethyl-1,3,2-dioxaborolan-2-yl)phenoxy)propan-2-amine

3-[(2S)-2-(Dimethylamino)propoxy]aniline (817 mg, 4.21 mmol) was dissolved in MeCN (11.25 mL) and bis(pinacolato) diboron (1.17 g, 4.63 mmol) was added thereto followed by addition of tert-butyl nitrite, tech. 90% (650.50 mg, 6.31 mmol, 750.28 μL). The resulting mixture was heated at 80° C. (oil bath) overnight. 12% of Starting material left by LCMS. Additional portions of tert-butyl nitrite, tech. 90% (650.50 mg, 6.31 mmol, 750.28 L) and bis(pinacolato) diboron (1.17 g, 4.63 mmol) was added to the reaction mixture and the resulting mixture was heated at 80° C. overnight. The reaction mixture was concentrated in vacuum. The residue was dissolved in DCM (50 ml) and the resulting solution was washed with 3% HCl solution (2*10 ml). The organic layer was dried over Na₂SO₄, filtered and concentrated in vacuum to obtain (2S)—N,N-dimethyl-1-[3-(4,4,5,5-tetramethyl-1,3,2-dioxaborolan-2-yl)phenoxy]propan-2-amine (1.62 g, crude).

LCMS(ESI): [M]⁺ m/z: calcd 305.2; found 306.2; Rt=0.901 min.

### Step 4: Synthesis of (S)-tert-butyl 6-(3-((S)-2-(dimethylamino)propoxy)phenyl)-3-methyl-3,4-dihydropyridine-1(2H)-carboxylate

Prepared by general procedure scheme 7.1 step 2. Yield: 0.16 g (8.05%).

CC conditions: The crude product was purified by silica gel with MTBE/MeOH 0-50% as an eluent mixture.

LCMS(ESI): [M]⁺ m/z: calcd 374.2; found 375.2; Rt=1.075 min.

### Step 5: Synthesis of (S)—N,N-dimethyl-1-(3-((S)-5-methyl-3,4,5,6-tetrahydropyridin-2-yl)phenoxy)propan-2-amine

Prepared by general procedure scheme 7.1 step 3. Yield: 152 mg of crude.

LCMS(ESI): [M]⁺ m/z: calcd 274.2; found 275.2; Rt=0.414 min.

### Step 6: Synthesis of (S)—N,N-dimethyl-1-(3-((2R,5S)-5-methylpiperidin-2-yl)phenoxy)propan-2-amine

Prepared by general procedure scheme 7.1 step 4. Yield: 121 mg of crude.

LCMS(ESI): [M]⁺ m/z: calcd 276.2; found 277.2; Rt=0.666 min.

### Intermediate 1. 2-Amino-3-methyl-quinoline-6-carboxylic acid

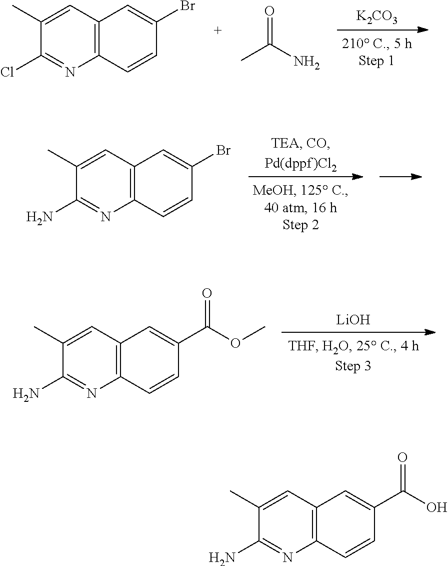

### Step 1: The Synthesis of 6-Bromo-3-methyl-quinolin-2-amine

Mixture of 6-bromo-2-chloro-3-methyl-quinoline (10 g, 38.98 mmol), acetamide (40.00 g, 677.19 mmol) and potassium carbonate, anhydrous, 99% (30.00 g, 217.07 mmol, 13.10 mL) was stirred at 210° C. for 5 hr. After cooling to room temperature, the reaction mixture was poured into water. A formed precipitate was collected by filtration, washed with water and dried at 70° C. overnight to give 6-bromo-3-methyl-quinolin-2-amine (12 g, crude).

LCMS(ESI): [M+3H]⁺ m/z: calcd 239.0; found 239.0; Rt=0.863 min.

### Step 2: The Synthesis of Methyl 2-amino-3-methyl-quinoline-6-carboxylate

Crude 6-bromo-3-methyl-quinolin-2-amine (18 g, 75.92 mmol), TEA (9.22 g, 91.10 mmol, 12.70 mL) and [1,1′-Bis(diphenylphosphino)ferrocene]dichloropalladium(II) (2.28 mmol) were dissolved in MeOH (300 mL). The reaction mixture was stirred at 125° C. and 40 atm for 16 hr in Carbon oxide (2.28 g, 75.92 mmol, 2.11 mL) atmosphere. The solvent was evaporated and the mixture was poured into water (200 ml) and extracted with EtOAc (2*200 mL). Combined organic layers were dried over Na₂SO₄ and evaporated to give methyl 2-amino-3-methyl-quinoline-6-carboxylate (5.2 g, 24.05 mmol, 31.68% yield).

¹H NMR (dmso, 400 MHz): δ (ppm) 2.27 (s, 3H), 3.91 (s, 3H), 6.78 (s, 2H), 7.52 (m, 1H), 7.93-7.99 (m, 2H), 8.32 (s, 1H).

LCMS(ESI): [M+H]⁺ m/z: calcd 217.1; found 218.2; Rt=0.812 min.

### Step 3: The Synthesis of 2-Amino-3-methyl-quinoline-6-carboxylic acid

To the stirring solution of methyl 2-amino-3-methyl-quinoline-6-carboxylate (5.2 g, 24.05 mmol) in THF (20 mL)/H₂O (15 mL) Lithium hydroxide monohydrate, 98% (2.32 g, 55.31 mmol, 1.54 mL) was added and the resulting mixture was stirred at 25° C. for 4 hr. THF was evaporated. The residue (water solution) was acidified with Sodium bisulfate to slightly acidic pH. Product was extracted with EtOAc (2*100 mL), dried over Na₂SO₄. EtOAc was evaporated to give 2-amino-3-methyl-quinoline-6-carboxylic acid (3.7 g, 18.30 mmol, 76.09% yield).

¹H NMR (dmso, 400 MHz): δ (ppm) 2.27 (s, 3H), 6.78 (s, 2H), 7.52-7.55 (d, 1H), 7.93 (s, 1H), 7.96-7.99 (d, 1H), 8.32 (s, 1H).

LCMS(ESI): [M+H]⁺ m/z: calcd 203.2; found 203.2; Rt=0.564 min.

### Example 1. Compound 95 N-(6-amino-5-ethylpyridin-3-yl)-2-((2R,5S)-5-methyl-2-(3-(1-methylpiperidin-4-yl)quinolin-7-yl)piperidin-1-yl)-2-oxoacetamide

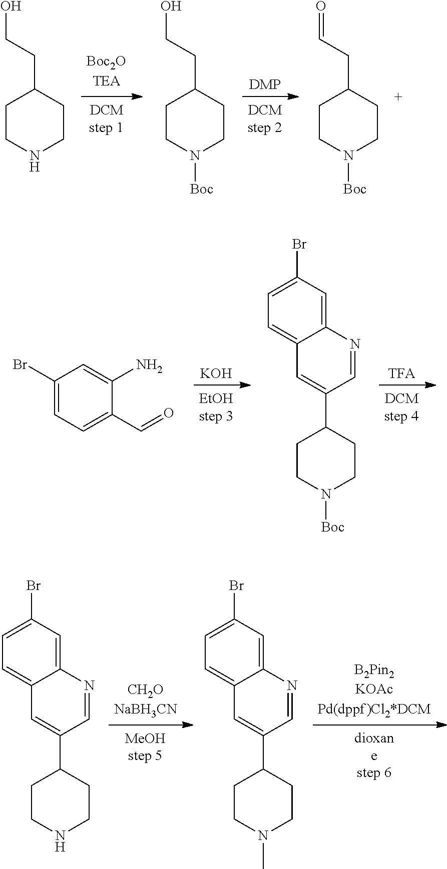

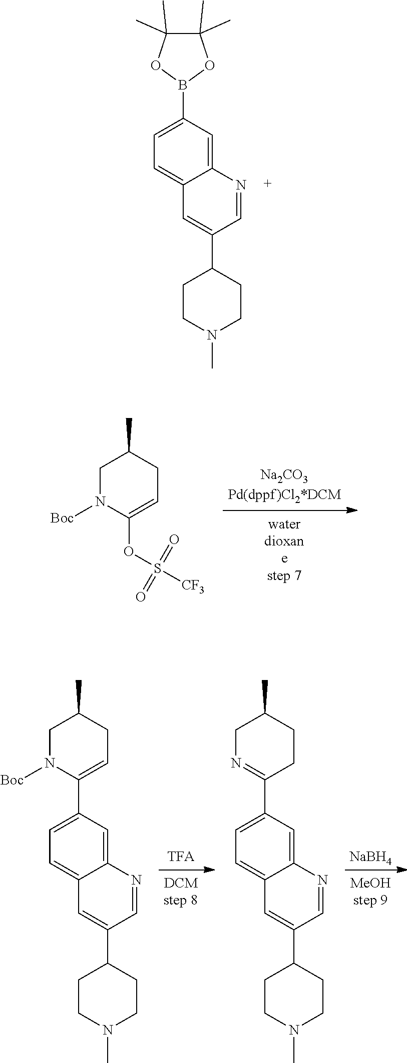 [SMILES: CN1CCC(c2cnc3cc(B4OC(C)(C)C(C)(C)O4)ccc3c2)CC1 ; C[C@H]1CC=C(OS(=O)(=O)C(F)(F)F)N(C(=O)OC(C)(C)C)C1 ; C[C@H]1CC=C(c2ccc3cc(C4CCN(C)CC4)cnc3c2)N(C(=O)OC(C)(C)C)C1 ; C[C@H]1CCC(c2ccc3cc(C4CCN(C)CC4)cnc3c2)=NC1 ; "NaBH4"]

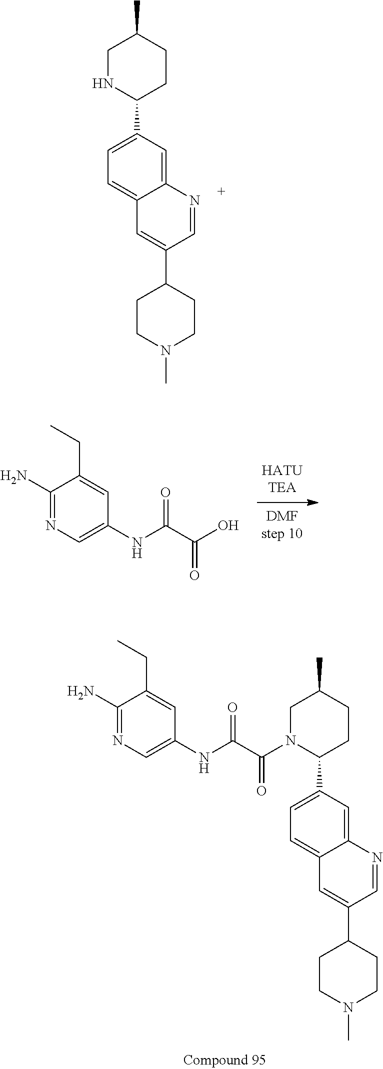 [SMILES: C[C@H]1CC[C@H](c2ccc3cc(C4CCN(C)CC4)cnc3c2)NC1 ; CCc1cc(NC(=O)C(=O)O)cnc1N ; CCc1cc(NC(=O)C(=O)N2C[C@@H](C)CC[C@@H]2c2ccc3cc(C4CCN(C)CC4)cnc3c2)cnc1N]

### Step 1: Synthesis of tert-butyl 4-(2-hydroxyethyl)piperidine-1-carboxylate

Di-tert-butyl dicarbonate (4.29 g, 19.65 mmol, 4.51 mL) was added dropwise to a solution of 2-(4-piperidyl)ethanol (3.1 g, 18.71 mmol, HCl) and TEA (4.73 g, 46.78 mmol, 6.52 mL) in DCM (30 mL). Resulting solution was stirred for 14 hr at 26° C. and diluted with water. The organic layer was separated and dried over Na₂SO₄, filtered and evaporated to obtain tert-butyl 4-(2-hydroxyethyl)piperidine-1-carboxylate (3.76 g, crude).

¹H NMR (400 MHz, CDCl₃) δ (ppm) 1.01 (m, 2H), 1.27 (s, 1H), 1.45 (s, 9H), 1.52 (m, 2H), 1.61 (m, 2H), 2.55 (m, 1H), 2.72 (m, 2H), 3.69 (m, 2H), 4.09 (m, 2H).

### Step 2: Synthesis of tert-butyl 4-(2-oxoethyl)piperidine-1-carboxylate

To a solution of tert-butyl 4-(2-hydroxyethyl)piperidine-1-carboxylate (3.76 g, 16.40 mmol) in DCM (100 mL) was added Dess-Martin periodinane (8.35 g, 19.68 mmol) and the reaction mixture was stirred at 25° C. for 14 hr. The reaction mixture was evaporated; 100 ml of water and 50 ml of MTBE were added. The precipitate formed was filtered off. The organic layer was separated and the aqueous was extracted with 2*50 ml MTBE. The organic layers were combined and washed with aq·NaHCO₃ (2*100 ml), then dried over Na₂SO₄ and concentrated to afford complicated mixture tert-butyl 4-(2-oxoethyl)piperidine-1-carboxylate (3.76 g, crude) which was used in the next step.

¹H NMR (500 MHz, CDCl₃) δ (ppm) 1.44 (s, 9H), 1.58 (m, 2H), 2.03 (m, 1H), 2.37 (m, 2H), 2.72 (m, 2H), 3.20 (m, 2H), 4.04 (m, 2H), 9.77 (m, 1H).

### Step 3: Synthesis of tert-butyl 4-(7-bromoquinolin-3-yl)piperidine-1-carboxylate

A solution of potassium hydroxide (167.86 mg, 2.99 mmol, 82.12 μL) in EtOH (50 mL) was added drop wise to a mixture of tert-butyl 4-(2-oxoethyl)piperidine-1-carboxylate (2 g, 8.80 mmol) and 2-amino-4-bromo-benzaldehyde (1.76 g, 8.80 mmol) in EtOH (50 mL). The mixture was heated to reflux, and then maintained at reflux for 3 hr. After cooling to room temperature, the reaction mixture was concentrated to remove the EtOH, then water was added and the mixture was extracted with DCM (3×50 ml) The combined organic layers were washed with water, dried over sodium sulfate, and concentrated in vacuum to give crude product which was purified by column chromatography (Interchim 80 g SiO₂, Hexane-MTBE from 0˜100%, flow rate=70 mL/min, cv=49.8) to give tert-butyl 4-(7-bromo-3-quinolyl)piperidine-1-carboxylate (1.5 g, 3.83 mmol, 43.57% yield).

LCMS(ESI): [M]⁺ m/z: calcd 391.2; found 392.2; Rt=1.572 min.

### Step 4: Synthesis of 7-bromo-3-(piperidin-4-yl)quinoline

tert-Butyl 4-(7-bromo-3-quinolyl)piperidine-1-carboxylate (1.4 g, 3.58 mmol) was dissolved in a mixture of DCM (5 mL) and TFA (5 mL). The resulting solution was stirred for 16 hr at 22° C. The reaction mixture was concentrated on vacuum. The obtained residue was dissolved in water, basified with NaHCO₃ and extracted with DCM (3 times), combined DCM layers were dried over Na₂SO₄, filtered and evaporated to give 7-bromo-3-(4-piperidyl)quinoline (0.7 g, 2.40 mmol, 67.19% yield). The obtained product was used in the next step without further purification.

LCMS(ESI): [M]⁺ m/z: calcd 291.2; found 292.2; Rt=0.896 min.

### Step 5: Synthesis of 7-bromo-3-(1-methylpiperidin-4-yl)quinoline

Formaldehyde, 37% w/w aq. soln., stab. with 7-8% MeOH (836.19 mg, 10.30 mmol, 772.10 μL, 37% purity) was added to a mixture of 7-bromo-3-(4-piperidyl)quinoline (0.6 g, 2.06 mmol) and sodium cyan borohydride (647.42 mg, 10.30 mmol) in MeOH (19.51 mL) and stirred overnight, then concentrated. The residue was treated with aq. solution of NaHCO₃ and desired product was extracted with 2*30 ml of DCM, dried over Na₂SO₄ and concentrated in vacuum to give 7-bromo-3-(1-methyl-4-piperidyl)quinoline (0.53 g, crude).

LCMS(ESI): [M]⁺ m/z: calcd 305.2; found 306.2; Rt=0.803 min.

### Step 6: Synthesis of 3-(1-methylpiperidin-4-yl)-7-(4,4,5,5-tetramethyl-1,3,2-dioxaborolan-2-yl)quinoline

Prepared by general procedure scheme 7.1 step 1. Yield: 0.45 g of crude.

LCMS(ESI): [M]⁺ m/z: calcd 352.2; found 353.2; Rt=0.869 min.

### Step 7: Synthesis of (S)-tert-butyl 3-methyl-6-(3-(1-methylpiperidin-4-yl)quinolin-7-yl)-3,4-dihydropyridine-1(2H)-carboxylate

Prepared by general procedure scheme 7.1 step 2. Yield: 0.6 g of crude.

LCMS(ESI): [M]⁺ m/z: calcd 421.2; found 422.2; Rt=1.023 min.

### Step 8: Synthesis of (S)-7-(5-methyl-3,4,5,6-tetrahydropyridin-2-yl)-3-(1-methylpiperidin-4-yl)quinoline

Prepared by general procedure scheme 7.1 step 3. Yield: 0.3 g of crude.

LCMS(ESI): [M]⁺ m/z: calcd 321.2; found 322.2; Rt=0.651 min.

### Step 9: Synthesis of 7-((2R,5S)-5-methylpiperidin-2-yl)-3-(1-methylpiperidin-4-yl)quinoline

Prepared by general procedure scheme 7.1 step 4. Yield: 0.21 g of crude.

LCMS(ESI): [M]⁺ m/z: calcd 323.2; found 324.2; Rt=0.502 min.

### Step 10: Synthesis of N-(6-amino-5-ethylpyridin-3-yl)-2-((2R,5S)-5-methyl-2-(3-(1-methylpiperidin-4-yl)quinolin-7-yl)piperidin-1-yl)-2-oxoacetamide (Compound 95)

Prepared by general procedure scheme 7.1 step 5A. Yield: 9.6 mg (2.64%).

HPLC conditions: Column: SunFire C18 100*19 mm, 5 microM; 0.6-8.6 min 0-100% MeCN+FA 30 ml/min; (loading pump 4 ml/min MeCN).

Compound 95: ¹H NMR (600 MHz, DMSO-d₆) δ (ppm) 1.03-1.38 (m, 8H), 1.70-1.85 (m, 4H), 2.07-2.40 (m, 8H), 2.72-2.95 (m, 4H), 3.51-4.08 (m, 2H), 5.37-5.77 (m, 4H), 7.43-7.60 (m, 2H), 7.88-8.15 (m, 4H), 8.83 (s, 1H), 10.54-10.59 (m, 1H).

LCMS(ESI): [M]⁺ m/z: calcd 514.2; found 515.2; Rt=1.892 min.

### Example 2. Compound 46 2-methoxy-5-(2-((2R,5S)-5-methyl-2-(2-(1-methyl-2,5,6,7-tetrahydro-1H-azepin-4-yl)benzo[d]thiazol-5-yl)piperidin-1-yl)-2-oxoacetamido)nicotinamide

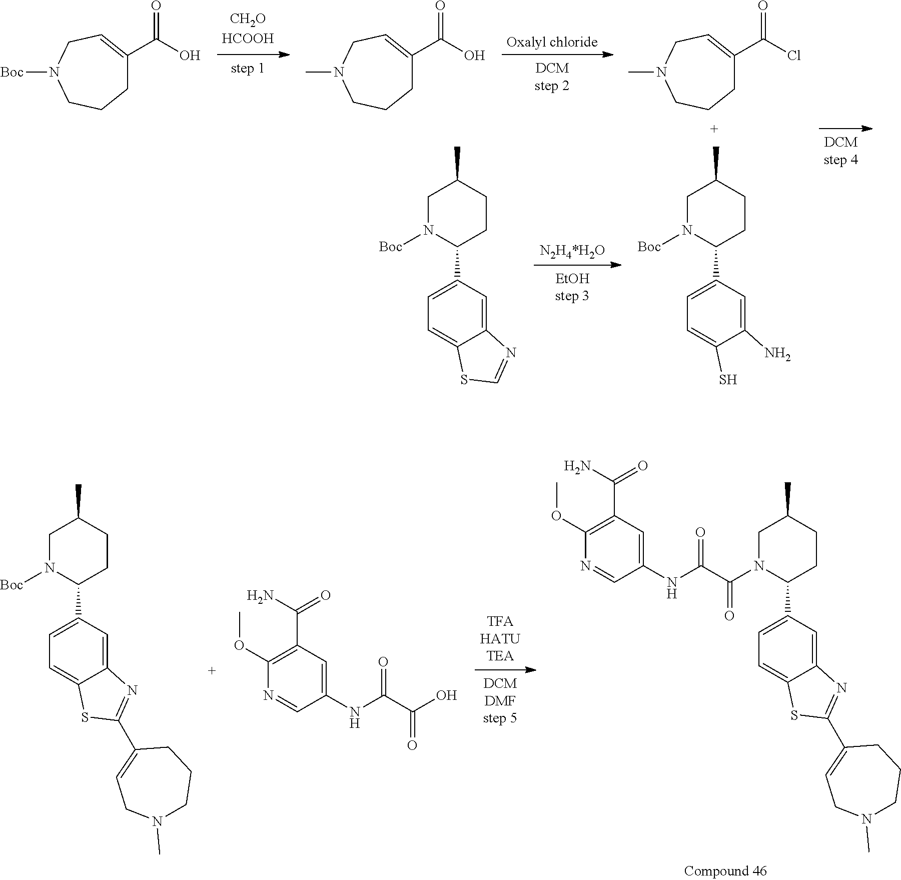

### Step 1: Synthesis of 1-methyl-2,5,6,7-tetrahydro-1H-azepine-4-carboxylic acid

Formaldehyde, 37% w/w aq. soln., stab. with 7-8% MeOH (672.67 mg, 8.29 mmol, 621.11 μL, 37% purity) was added to the solution of 1-tert-butoxycarbonyl-2,3,4,7-tetrahydroazepine-5-carboxylic acid (800 mg, 3.32 mmol) in formic acid (12.20 g, 225.31 mmol, 10 mL, 85% purity). Resulting mixture was stirred at 80° C. for 14 hr. Then, volatiles were removed under reduced pressure, leaving 1-methyl-2,3,4,7-tetrahydroazepine-5-carboxylic acid (0.8 g, crude).

LCMS(ESI): [M]⁺ m/z: calcd 155.2; found 156.2; Rt=0.188 min.

### Step 2: Synthesis of 1-methyl-2,5,6,7-tetrahydro-1H-azepine-4-carbonyl chloride

Oxalyl chloride (3.27 g, 25.77 mmol, 2.25 mL) was added dropwise to the solution of 1-methyl-2,3,4,7-tetrahydroazepine-5-carboxylic acid (0.8 g, 5.15 mmol) in DCM (20 mL). Resulting mixture was stirred at 25° C. for 3 hr. Then, solvent was removed under reduced pressure. Residue was dissolved in chloroform (20 ml) and concentrated in vacuum again, affording 1-methyl-2,3,4,7-tetrahydroazepine-5-carbonyl chloride (0.95 g, 4.52 mmol, 87.72% yield, HCl).

¹H NMR (500 MHz, CDCl₃) δ (ppm) 1.49 (m, 1H), 2.05 (m, 1H), 2.21 (m, 1H), 2.83 (s, 3H), 2.92 (m, 1H), 3.33 (m, 1H), 3.76 (m, 1H), 4.28 (m, 2H), 8.02 (m, 1H).

### Step 3: Synthesis of (2R,5S)-tert-butyl 2-(3-amino-4-mercaptophenyl)-5-methylpiperidine-1-carboxylate

Hydrazine hydrate (6.32 g, 126.33 mmol, 6.16 mL) was added to the solution of tert-butyl (2R,5S)-2-(1,3-benzothiazol-5-yl)-5-methyl-piperidine-1-carboxylate (4.2 g, 12.63 mmol) in EtOH (50 mL). Resulting mixture was stirred at 78° C. for 72 hr. Then, volatiles were removed under reduced pressure and residue was diluted with water (50 ml) and extracted with MTBE (2×40 ml). Combined organic layers were dried over Na₂SO₄ and concentrated under reduced pressure, leaving tert-butyl (2R,5S)-2-(3-amino-4-sulfanyl-phenyl)-5-methyl-piperidine-1-carboxylate (4 g, 12.40 mmol, 98.19% yield).

LCMS(ESI): [M(disulfide)-Boc]⁺ m/z: calcd 542.2; found 543.2; Rt=1.800 min.

### Step 4: Synthesis of (2R,5S)-tert-butyl 5-methyl-2-(2-(1-methyl-2,5,6,7-tetrahydro-1H-azepin-4-yl)benzo[d]thiazol-5-yl)piperidine-1-carboxylate

1-Methyl-2,3,4,7-tetrahydroazepine-5-carbonyl chloride (274 mg, 1.30 mmol, HCl) solution in DCM (2 mL) was added dropwise to the solution of tert-butyl (2R,5S)-2-(3-amino-4-sulfanyl-phenyl)-5-methyl-piperidine-1-carboxylate (420.54 mg, 1.30 mmol) in DCM (3 mL) under argon. Resulting mixture was stirred at 25° C. for 24 hr. Then, volatiles were removed under reduced pressure and residue was subjected to HPLC (50-50-100% 0-1-6 min H₂O/MeCN/0.1% NH₄OH, flow: 30 ml/min; column: XBridge BEH C18 100×19 mm, 5 um), affording tert-butyl (2R,5S)-5-methyl-2-[2-(1-methyl-2,3,4,7-tetrahydroazepin-5-yl)-1,3-benzothiazol-5-yl]piperidine-1-carboxylate (25 mg, 56.61 mol, 4.34% yield).

LCMS(ESI): [M]⁺ m/z: calcd 441.2; found 442.2; Rt=1.264 min.

### Step 5: Synthesis of 2-methoxy-5-(2-((2R,5S)-5-methyl-2-(2-(1-methyl-2,5,6,7-tetrahydro-1H-azepin-4-yl)benzo[d]thiazol-5-yl)piperidin-1-yl)-2-oxoacetamido)nicotinamide (Compound 46)

TFA (148.00 mg, 1.30 mmol, 0.1 mL) was added to the solution of tert-butyl (2R,5S)-5-methyl-2-[2-(1-methyl-2,3,4,7-tetrahydroazepin-5-yl)-1,3-benzothiazol-5-yl]piperidine-1-carboxylate (25 mg, 56.61 mol) in DCM (1 mL). Resulting mixture was stirred at 25° C. for 3 hr. Then, volatiles were removed under reduced pressure and residue was dissolved in DMF (1 mL). 2-[(5-Carbamoyl-6-methoxy-3-pyridyl)amino]-2-oxo-acetic acid (17.60 mg, 73.59 μmol) and TEA (57.28 mg, 566.09 mol, 78.90 μL) were added to this solution followed by HATU (30.13 mg, 79.25 μmol). The resulting reaction mixture was stirred at 25° C. for 3 hr. Then, it was subjected to HPLC (40-40-90% 0-1-6 min H₂O/MeOH/0.1% NH₄OH, flow: 30 ml/min; column: XBridge BEH C18 100×19 mm, 5 um), affording 2-methoxy-5-[[2-oxo-2-[(2R,5S)-5-methyl-2-[2-(1-methyl-2,3,4,7-tetrahydroazepin-5-yl)-1,3-benzothiazol-5-yl]-1-piperidyl]acetyl]amino]pyridine-3-carboxamide (13 mg, 23.10 mol, 40.811% yield).

Compound 46: ¹H NMR (DMSO-d6, 600 MHz): δ (ppm) 1.02-1.06 (m, 3H), 1.32-1.40 (m, 1H), 1.68-1.92 (m, 4H), 2.07-2.31 (m, 4H), 2.45 (m, 2H), 2.82 (m, 2H), 3.49-4.05 (m, 5H), 5.27-5.69 (m, 1H), 6.94-6.96 (m, 1H), 7.34-7.41 (d, 1H), 7.68 (d, 1H), 7.75 (s, 1H), 7.85 (m, 1H), 7.99-8.03 (d, 1H), 8.41-8.58 (m, 2H), 11.11 (s, 1H).

LCMS(ESI): [M]⁺ m/z: calcd 562.2; found 563.2; Rt=2.806 min.

### Example 3. Compound 5 2-methoxy-5-[[2-oxo-2-[(2R,5S)-5-methyl-2-[2-[1-(trideuteriomethyl)-4-piperidyl]-1,3-benzothiazol-5-yl]-1-piperidyl]acetyl]amino]pyridine-3-carboxamide

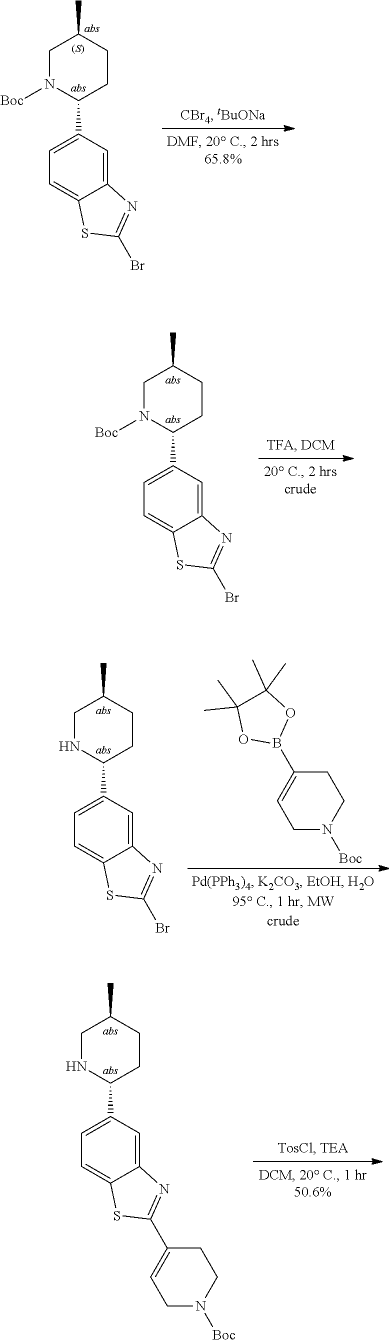

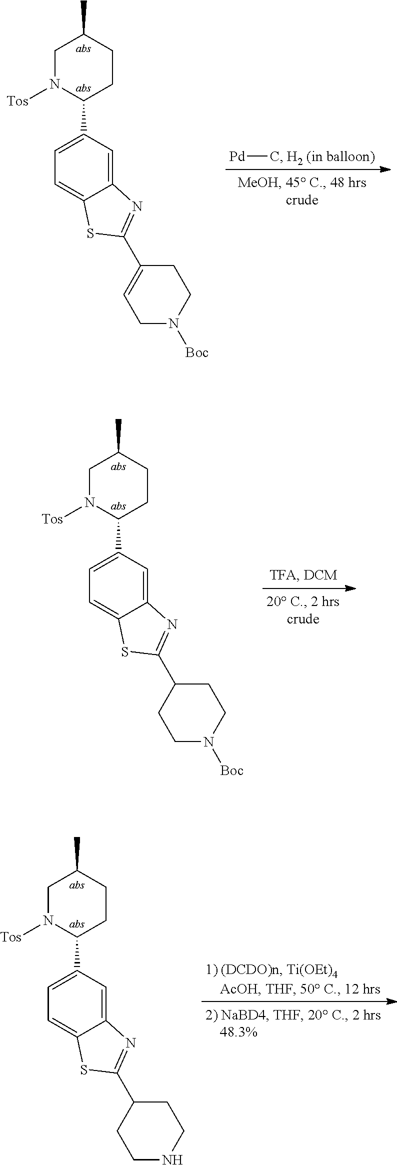

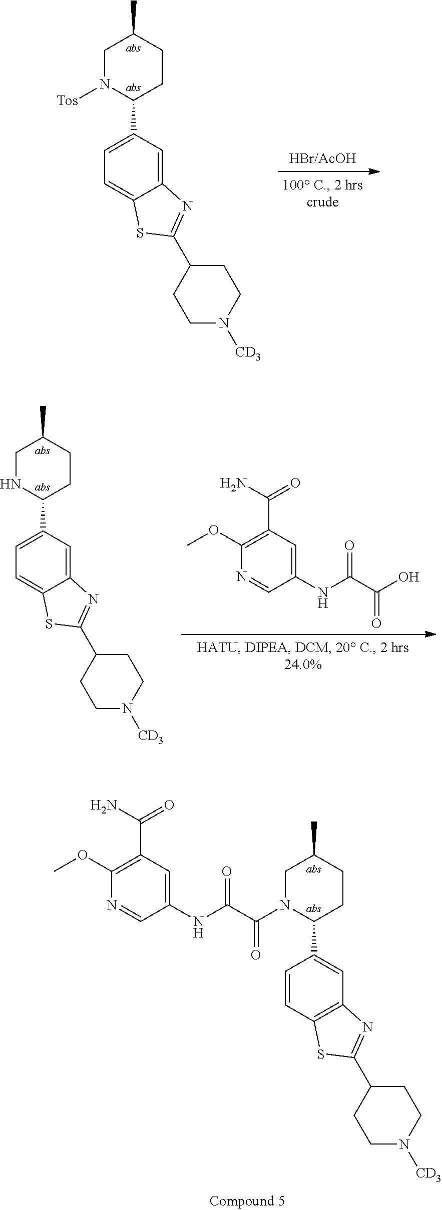

### Step 1: Synthesis of tert-butyl (2R,5S)-2-(2-bromo-1,3-benzothiazol-5-yl)-5-methyl-piperidine-1-carboxylate

To a mixture of tert-butyl (2R,5S)-2-(1,3-benzothiazol-5-yl)-5-methyl-piperidine-1-carboxylate (4.30 g, 12.9 mmol), CBr₄ (4.30 g, 13.0 mmol) and DMF (20 mL) was added sodium; 2-methylpropan-2-olate (5.2 g, 54.1 mmol) and the mixture was stirred at 20° C. for 2 hours. The resulting mixture was quenched by addition of water (50 mL) and extracted with EtOAc (50 mL*3). The combined organic layer was washed with saturated NH₄C₁ aqueous solution (50 mL*2), brine (50 mL), dried over anhydrous Na₂SO₄, filtered and concentrated under reduced pressure to give tert-butyl (2R,5S)-2-(2-bromo-1,3-benzothiazol-5-yl)-5-methyl-piperidine-1-carboxylate (3.5 g, 65.8% yield) as yellow oil. LCMS (ESI) [M+H]⁺ m/z calcd 411.1, found 355.0 (t-Bu cleaved mass).

### Step 2: Synthesis of 2-bromo-5-[(2R,5S)-5-methyl-2-piperidyl]-1,3-benzothiazole

A mixture of tert-butyl (2R,5S)-2-(2-bromo-1,3-benzothiazol-5-yl)-5-methyl-piperidine-1-carboxylate (1.00 g, 2.43 mmol), DCM (9 mL) and TFA (2 mL, 26.0 mmol,) was stirred at 20° C. for 2 hours. The resulting mixture was adjusted to pH=9 with saturated Na₂CO₃ aqueous solution, and then the mixture was extracted with DCM (50 mL*3). The combined organic layer was washed with brine (50 mL), dried over anhydrous Na₂SO₄, filtered and concentrated under reduced pressure to give 2-bromo-5-[(2R,5S)-5-methyl-2-piperidyl]-1,3-benzothiazole (750 mg, crude) as yellow solid. LCMS (ESI) [M+H]⁺ m/z calcd 313.0, found 312.9.

### Step 3: Synthesis of tert-butyl 4-[5-[(2R,5S)-5-methyl-2-piperidyl]-1,3-benzothiazol-2-yl]-3,6-dihydro-2H-pyridine-1-carboxylate

To a mixture of 2-bromo-5-[(2R,5S)-5-methyl-2-piperidyl]-1,3-benzothiazole (650 mg, 2.09 mmol) and tert-butyl 4-(4,4,5,5-tetramethyl-1,3,2-dioxaborolan-2-yl)-3,6-dihydro-2H-pyridine-1-carboxylate (850 mg, 2.75 mmol) in EtOH (12 mL) and H₂O (5 mL) were added Pd(PPh₃)₄ (260 mg, 0.225 mmol) and K₂CO₃ (910 mg, 6.58 mmol). The resulting mixture was stirred at 95° C. for 1 hour under microwave. The resulting mixture was concentrated under reduced pressure. The residue was purified by flash chromatography (ISCO®; 20 g AgelaFlash® Silica Flash Column, DCM/MeOH with MeOH from 0˜10%, Flow Rate: 30 mL/min, 254 nm) to afford tert-butyl 4-[5-[(2R,5S)-5-methyl-2-piperidyl]-1,3-benzothiazol-2-yl]-3,6-dihydro-2H-pyridine-1-carboxylate (1.0 g, crude) as yellow oil. LCMS (ESI) [M+H]⁺ m/z calcd 414.2, found 414.1.

### Step 4: Synthesis of tert-butyl 4-[5-[(2R,5S)-5-methyl-1-(p-tolylsulfonyl)-2-piperidyl]-1,3-benzothiazol-2-yl]-3,6-dihydro-2H-pyridine-1-carboxylate

To a mixture of tert-butyl 4-[5-[(2R,5S)-5-methyl-2-piperidyl]-1,3-benzothiazol-2-yl]-3,6-dihydro-2H-pyridine-1-carboxylate (1.08 g, 2.61 mmol), TEA (1.08 mL, 7.75 mmol) in DCM (10 mL) was added 4-methylbenzenesulfonyl chloride (605 mg, 3.17 mmol) slowly. The resulting mixture was stirred at 20° C. for 1 hour. The resulting mixture was quenched by addition of water (50 mL) and extracted with DCM (100 mL*3). The combined organic layer was washed with saturated NH₄C₁ aqueous solution (50 mL), brine (100 mL), dried over anhydrous Na₂SO₄, filtered and concentrated under reduced pressure. The residue was purified by flash chromatography (ISCO®; 20 g AgelaFlash® Silica Flash Column, petroleum ether/EtOAc with EtOAc from 0˜20%, flow rate=30 mL/min, 254 nm) to afford tert-butyl 4-[5-[(2R,5S)-5-methyl-1-(p-tolylsulfonyl)-2-piperidyl]-1,3-benzothiazol-2-yl]-3,6-dihydro-2H-pyridine-1-carboxylate (750 mg, 50.6% yield) as white solid. LCMS (ESI) [M+H]⁺ m/z: calcd 568.2, found 568.3.

### Step 5: Synthesis of tert-butyl 4-[5-[(2R,5S)-5-methyl-1-(p-tolylsulfonyl)-2-piperidyl]-1,3-benzothiazol-2-yl]piperidine-1-carboxylate

A mixture of tert-butyl 4-[5-[(2R,5S)-5-methyl-1-(p-tolylsulfonyl)-2-piperidyl]-1,3-benzothiazol-2-yl]-3,6-dihydro-2H-pyridine-1-carboxylate (710 mg, 1.25 mmol) and Pd/C (600 mg, 10 wt % Pd with 50 wt % water) in MeOH (10 mL) was stirred at 45° C. for 48 hours under Hydrogen (in balloon). The resulting mixture was filtered and concentrated under reduced pressure to give tert-butyl 4-[5-[(2R,5S)-5-methyl-1-(p-tolylsulfonyl)-2-piperidyl]-1,3-benzothiazol-2-yl]piperidine-1-carboxylate (600 mg, crude) as a yellow solid. LCMS (ESI) [M+H]⁺ m/z: calcd 570.2, found 514.2 (t-Bu cleaved mass).

### Step 6: Synthesis of 2-(4-piperidyl)-5-[(2R,5S)-5-methyl-1-(p-tolylsulfonyl)-2-piperidyl]-1,3-benzothiazole

A mixture of tert-butyl 4-[5-[(2R,5S)-5-methyl-1-(p-tolylsulfonyl)-2-piperidyl]-1,3-benzothiazol-2-yl]piperidine-1-carboxylate (600 mg, 0.906 mmol), DCM (6 mL) and TFA (0.7 mL, 9.09 mmol) was stirred at 20° C. for 2 hours. The resulting mixture was adjusted to pH=8 with NH₃—H₂O (12 N). The resulting mixture was quenched by addition of water (50 mL) and extracted with DCM (100 mL*3). The combined organic layer was washed with brine (100 mL), dried over anhydrous Na₂SO₄, filtered and concentrated under reduced pressure to give 2-(4-piperidyl)-5-[(2R,5S)-5-methyl-1-(p-tolylsulfonyl)-2-piperidyl]-1,3-benzothiazole (600 mg, crude) as yellow solid, which was used to the next step without further purification. LCMS (ESI) [M+H]⁺ m/z: calcd 470.2, found 470.3.

### Step 7: Synthesis of 5-[(2R,5S)-5-methyl-1-(p-tolylsulfonyl)-2-piperidyl]-2-[1-(trideuteriomethyl)-4-piperidyl]-1,3-benzothiazole

A mixture of 2-(4-piperidyl)-5-[(2R,5S)-5-methyl-1-(p-tolylsulfonyl)-2-piperidyl]-1,3-benzothiazole (600 mg, 1.28 mmol) in THF (15 mL) were added AcOH (0.05 mL, 1.28 mmol), (DCDO)ₙ (223 mg, 6.36 mmol) and ethanolate; titanium(4+) (7.89 mmol, 1.65 mL). the resulting mixture was stirred at 50° C. for 12 hours. NaBD₄ (90 mg, 2.26 mmol) was added. The mixture was stirred at 20° C. for 2 hours. The resulting mixture was quenched by addition of water (50 mL) and extracted with EtOAc (100 mL*3). The combined organic layer was washed with brine (50 mL), dried over anhydrous Na₂SO₄, filtered and concentrated under reduced pressure. The residue was purified by flash chromatography (ISCO®; 12 g AgelaFlash® Silica Flash Column, DCM/MeOH with MeOH from 0˜1^{3%}, flow rate=30 mL/min, 254 nm) to afford 5-[(2R,5S)-5-methyl-1-(p-tolylsulfonyl)-2-piperidyl]-2-[1-(trideuteriomethyl)-4-piperidyl]-1,3-benzothiazole (300 mg, 48.3% yield) as white solid. LCMS (ESI) [M+H]⁺ m/z: calcd 487.2, found 487.1.

### Step 8: Synthesis of 5-[(2R,5S)-5-methyl-2-piperidyl]-2-[1-(trideuteriomethyl)-4-piperidyl]-1,3-benzothiazole

A mixture of 5-[(2R,5S)-5-methyl-1-(p-tolylsulfonyl)-2-piperidyl]-2-[1-(trideuteriomethyl)-4-piperidyl]-1,3-benzothiazole (300 mg, 0.616 mmol), HBr/AcOH (2 mL, 0.616 mmol, 33 wt %) was stirred at 100° C. for 2 hours. The resulting mixture was concentrated under reduced pressure to removed HBr/AcOH and adjusted to pH=9 with saturated Na₂CO₃ aqueous solution. The solution was stirred at 25° C. for 12 hours. The resulting mixture was filtered and concentrated under reduced pressure to afford 5-[(2R,5S)-5-methyl-2-piperidyl]-2-[1-(trideuteriomethyl)-4-piperidyl]-1,3-benzothiazole (200 mg, crude) as a yellow solid. LCMS (ESI) [M+H]⁺ m/z: calcd 333.2, found 333.2.

### Step 9: Synthesis of 2-methoxy-5-[[2-oxo-2-[(2R,5S)-5-methyl-2-[2-[1-(trideuteriomethyl)-4-piperidyl]-1,3-benzothiazol-5-yl]-1-piperidyl]acetyl]amino]pyridine-3-carboxamide Compound 5)

A mixture of 5-[(2R,5S)-5-methyl-2-piperidyl]-2-[1-(trideuteriomethyl)-4-piperidyl]-1,3-benzothiazole (150 mg, 0.451 mmol), 2-[(5-carbamoyl-6-methoxy-3-pyridyl)amino]-2-oxo-acetic acid (150 mg, 0.627 mmol), DMF (5 mL), HATU (210 mg, 0.552 mmol) and DIPEA (0.300 mL, 1.72 mmol) was stirred at 20° C. for 2 hours. The mixture was concentrated under reduced pressure to give a crude product, which was purified by preparative HPLC (Instrument: Gilson GX-281 Liquid Handler, Gilson 322 Pump, Gilson 156 UV Detector; Column: 2_Phenomenex Gemini Cis 75*40 mm*3 um; Mobile phase A: H₂O with 0.05% NH₃—H₂O (v %); Mobile phase B: MeCN; Gradient: B from 30% to 60% in 9.5 min, hold 100% B for 3 min; Flow Rate: 25 mL/min; Column Temperature: 30° C.; Wavelength: 220 nm, 254 nm) to afford product (80 mg, 90%, contained 10% single impurity) as a white solid. The mixture was purified by SFC (Instrument: Berger, MULTIGR AM-II; Column: DAICEL CHIRAL OD-H (250 mm*30 mm*5 um); Mobile phase: supercritical Hexane-IPA (0.1% NH₃, EtOH v %)=55/45; Flow Rate:80 mL/min; Column Temperature: 38° C.; Nozzle Pressure: 100 bar; Nozzle Temperature: 60° C.; Evaporator Temperature: 20° C.; Trimmer Temperature: 25° C.; Wavelength:220 nm). The fraction was concentrated under reduced pressure and then lyophilized for overnight to afford 2-methoxy-5-[[2-oxo-2-[(2R,5S)-5-methyl-2-[2-[1-(trideuteriomethyl)-4-piperidyl]-1,3-benzothiazol-5-yl]-1-piperidyl]acetyl]amino]pyridine-3-carboxamide (60 mg, 24.0% yield) as white solid. ¹H NMR (400 MHz, methanol-d₄) δ ppm 8.57-8.72 (m, 1H), 8.32-8.54 (m, 1H), 7.87-8.01 (m, 2H), 7.39-7.55 (m, 1H), 5.46-5.89 (m, 1H), 4.00-4.17 (m, 4H), 3.43-3.80 (m, 1H), 3.10-3.25 (m, 1H), 3.00 (br d, J=11.3 Hz, 2H), 2.16-2.37 (m, 6H), 1.88-2.02 (m, 4H), 1.47 (br d, J=11.8 Hz, 1H), 1.14 (d, J=6.8 Hz, 3H); LCMS (ESI) [M+H]⁺ m/z: calcd 554.3, found 554.3; HPLC: 96.30%@254 nm; SFC: 98.2% de.

### Example 4. Compound 109 N-(6-amino-5-ethyl-3-pyridyl)-2-oxo-2-[(2R,5S)-5-methyl-2-[2-(1,2,2-trimethyl-4-piperidyl)indazol-6-yl]-1-piperidyl]acetamide

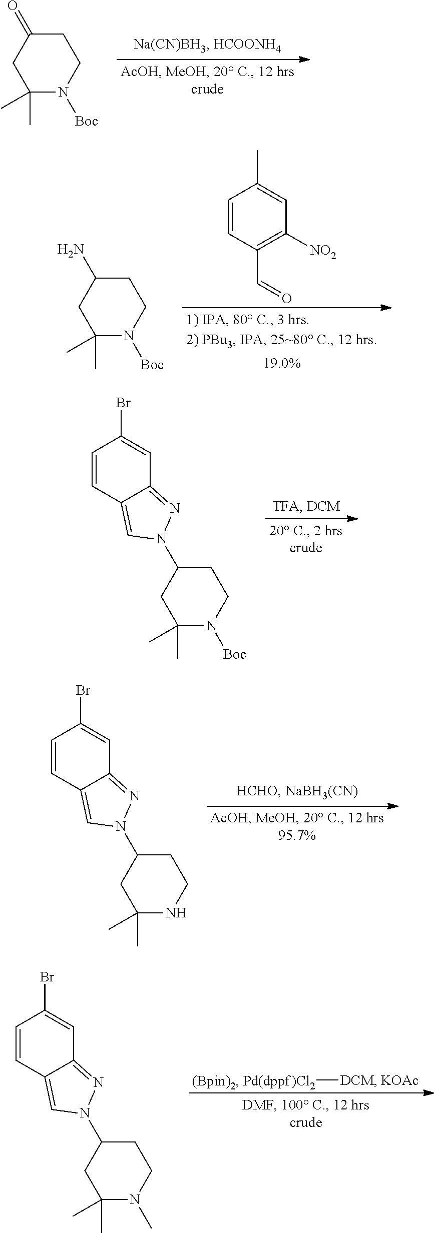

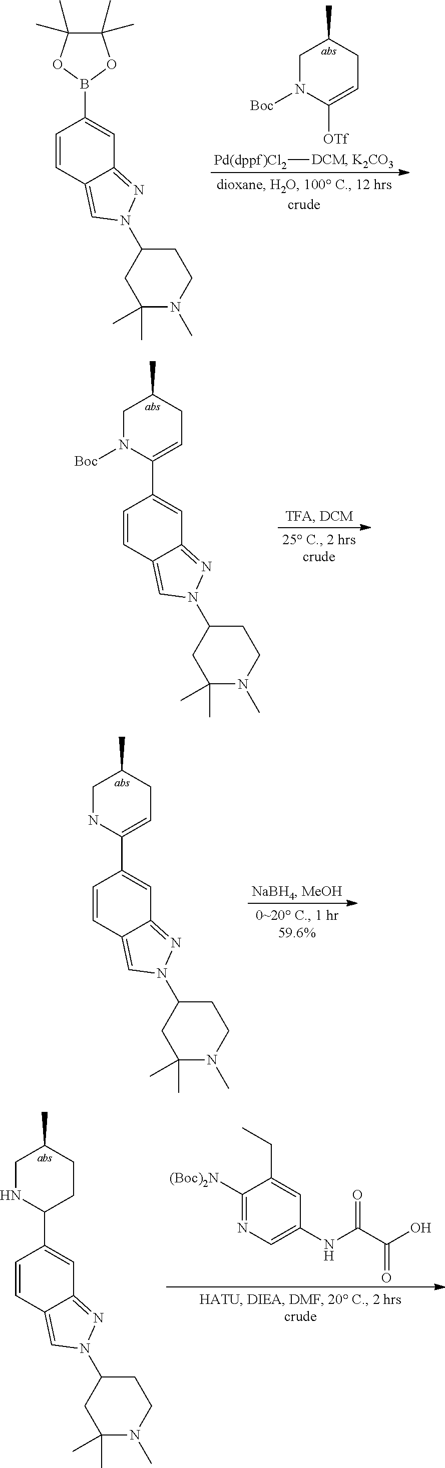

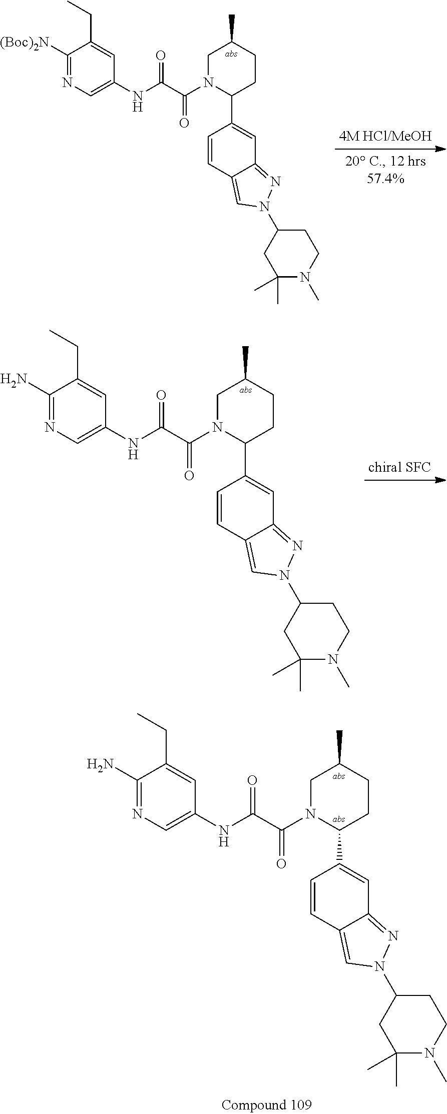

### Step 1: Synthesis of tert-butyl 4-amino-2,2-dimethyl-piperidine-1-carboxylate

To a solution of tert-butyl 2,2-dimethyl-4-oxo-piperidine-1-carboxylate (500 mg, 2.20 mmol) and HCOONH₄ (5.50 g, 87.2 mmol) in MeOH (10 mL) was added acetic acid (0.130 mL, 2.27 mmol) and Na(CN)BH₃ (280 mg, 4.46 mmol). The mixture was stirred at 20° C. for 12 hours. The mixture was concentrated under reduced pressure to remove MeOH. The residue was diluted with H₂O (100 mL), and extracted with EtOAc (100 mL*3). The combined organic layer was dried over anhydrous Na₂SO₄, filtered and concentrated under reduced pressure to give tert-butyl 4-amino-2,2-dimethyl-piperidine-1-carboxylate (500 mg, crude) as a colorless oil, which was directly used to next step without further purification. ¹H NMR (400 MHz, methanol-d₄) δ ppm 3.81-3.94 (m, 1H), 3.44 (dt, J=12.5, 4.5 Hz, 1H), 3.22-3.30 (m, 1H), 2.06-2.16 (m, 1H), 1.80-1.88 (m, 1H), 1.66-1.76 (m, 1H), 1.49-1.63 (m, 4H), 1.46 (s, 9H), 1.34-1.41 (m, 3H).

### Step 2: Synthesis of tert-butyl 4-(6-bromoindazol-2-yl)-2,2-dimethyl-piperidine-1-carboxylate

To a solution of tert-butyl 4-amino-2,2-dimethyl-piperidine-1-carboxylate (500 mg, 2.19 mmol) and 4-bromo-2-nitro-benzaldehyde (500 mg, 2.17 mmol) in IPA (10 mL) was stirred at 80° C. for 3 hours. Then tributylphosphane (1.62 mL, 6.57 mmol) was added to above mixture at 25° C., then the reaction mixture was stirred at 80° C. for 12 hours. The resulting mixture was quenched by addition of water (100 mL) and extracted with EtOAc (100 mL*3). The combined organic layer was washed with brine (100 mL), dried over anhydrous Na₂SO₄, filtered and concentrated under reduced pressure. The residue was purified by flash chromatography (ISCO®; 12 g AgelaFlash® Silica Flash Column, petroleum ether/EtOAc with EtOAc from 0˜15%, flow rate=30 mL/min, 254 nm) to afford tert-butyl 4-(6-bromoindazol-2-yl)-2,2-dimethyl-piperidine-1-carboxylate (170 mg, 19.0% yield) as a yellow oil. ¹H NMR (400 MHz, methanol-d₄) δ ppm 8.34 (d, J=0.8 Hz, 1H), 7.73-7.84 (m, 1H), 7.63 (d, J=8.8 Hz, 1H), 7.16 (dd, J=8.8, 1.5 Hz, 1H), 4.0-4.4 (m, 1H), 3.42-3.48 (m, 1H), 2.27-2.39 (m, 2H), 2.06-2.17 (m, 1H), 1.97-2.05 (m, 1H), 1.59 (s, 3H), 1.49 (s, 12H); LCMS (ESI) [M+H]⁺ m/z: calcd 408.1, found 408.1.

### Step 3: Synthesis of 6-bromo-2-(2,2-dimethyl-4-piperidyl)indazole

A mixture of tert-butyl 4-(6-bromoindazol-2-yl)-2,2-dimethyl-piperidine-1-carboxylate (200 mg, 0.490 mmol), DCM (2 mL) and TFA (2 mL, 26.0 mmol) was stirred at 20° C. for 2 hours. The resulting mixture was concentrated under reduced pressure to afford 6-bromo-2-(2,2-dimethyl-4-piperidyl)indazole (150 mg, crude) as a yellow oil.

### Step 4: Synthesis of 6-bromo-2-(1,2,2-trimethyl-4-piperidyl)indazole

To a mixture of 6-bromo-2-(2,2-dimethyl-4-piperidyl)indazole (150 mg, 0.487 mmol) in MeOH (5 mL) was adjusted to pH=7 with Na₂CO₃, then AcOH (0.02 mL, 0.350 mmol), HCHO (30 mg, 0.999 mmol) and Na(CN)BH₃ (90.0 mg, 1.43 mmol) was added. The resulting mixture was stirred at 20° C. for 12 hours. The resulting mixture was quenched by addition of water (50 mL) and extracted with EtOAc (100 mL*3). The combined organic layer was washed with brine (100 mL), dried over anhydrous Na₂SO₄, filtered and concentrated under reduced pressure. The residue was purified by flash chromatography (ISCO®; 12 g AgelaFlash® Silica Flash Column, DCM/MeOH with MeOH from 0˜13%, flow rate=30 mL/min, 254 nm) to afford 6-bromo-2-(1,2,2-trimethyl-4-piperidyl)indazole (150 mg, 95.7% yield) as colorless solid. LCMS (ESI) [M+H]⁺ m/z: calcd 324.1, found 324.0.

### Step 5: Synthesis of 6-(4,4,5,5-tetramethyl-1,3,2-dioxaborolan-2-yl)-2-(1,2,2-trimethyl-4-piperidyl)indazole

To a mixture of 6-bromo-2-(1,2,2-trimethyl-4-piperidyl)indazole (110 mg, 0.341 mmol) in DMF (3 mL) were added Pd(dppf)Cl₂-DCM (33.0 mg, 0.0404 mmol), KOAc (100 mg, 1.02 mmol) and 4,4,5,5-tetramethyl-2-(4,4,5,5-tetramethyl-1,3,2-dioxaborolan-2-yl)-1,3,2-dioxaborolane (130 mg, 0.512 mmol). The resulting mixture was sealed and degassed under vacuum and purged with N₂ for three times, and then stirred at 100° C. for 12 hours under nitrogen atmosphere. The resulting mixture was quenched by addition of water (30 mL) and extracted with EtOAc (50 mL*3). The combined organic layer was washed with brine (50 mL), dried over anhydrous Na₂SO₄, filtered and concentrated under reduced pressure. The residue was purified by flash chromatography (ISCO®; 4*2 g AgelaFlash® Silica Flash Column, DCM/MeOH with MeOH from 0˜13%, flow rate=30 mL/min, 254 nm) to afford 6-(4,4,5,5-tetramethyl-1,3,2-dioxaborolan-2-yl)-2-(1,2,2-trimethyl-4-piperidyl)indazole (130 mg, crude) as white solid. LCMS (ESI) [M+H]⁺ m/z: calcd 370.3, found 370.2.

### Step 6: Synthesis of tert-butyl (3S)-3-methyl-6-[2-(1,2,2-trimethyl-4-piperidyl)indazol-6-yl]-3,4-dihydro-2H-pyridine-1-carboxylate

A mixture of 6-(4,4,5,5-tetramethyl-1,3,2-dioxaborolan-2-yl)-2-(1,2,2-trimethyl-4-piperidyl)indazole (130 mg, 0.352 mmol), tert-butyl (3S)-3-methyl-6-(trifluoromethylsulfonyloxy)-3,4-dihydro-2H-pyridine-1-carboxylate (160 mg, 0.463 mmol), K₂CO₃ (146 mg, 1.06 mmol), Pd(dppf)Cl₂-DCM (40 mg, 49.0 mol) in dioxane (4 mL) and H₂O (1 mL) was stirred at 100° C. for 12 hours under nitrogen. The resulting mixture was concentrated under reduced pressure to give residue which was purified by flash chromatography (ISCO®; 12 g AgelaFlash® Silica Flash Column, DCM/MeOH with MeOH from 0˜14%, flow rate=30 mL/min, 254 nm) to afford tert-butyl (3S)-3-methyl-6-[2-(1,2,2-trimethyl-4-piperidyl)indazol-6-yl]-3,4-dihydro-2H-pyridine-1-carboxylate (70 mg, crude) as brown oil. LCMS (ESI) [M+H]⁺ m/z: calcd 439.3, found 439.3.

### Step 7: Synthesis of 6-[(3S)-3-methyl-2,3,4,5-tetrahydropyridin-6-yl]-2-(1,2,2-trimethyl-4-piperidyl)indazole

A mixture of tert-butyl (3S)-3-methyl-6-[2-(1,2,2-trimethyl-4-piperidyl)indazol-6-yl]-3,4-dihydro-2H-pyridine-1-carboxylate (70.0 mg, 0.160 mmol), DCM (1 mL) and TFA (1 mL, 13.0 mmol) was stirred at 25° C. for 2 hours. The resulting mixture was concentrated under reduced pressure to give 6-[(3S)-3-methyl-2,3,4,5-tetrahydropyridin-6-yl]-2-(1,2,2-trimethyl-4-piperidyl)indazole (50 mg, crude) as a yellow oil.

### Step 8: Synthesis of 6-[(5S)-5-methyl-2-piperidyl]-2-(1,2,2-trimethyl-4-piperidyl)indazole

To a mixture of 6-[(3S)-3-methyl-2,3,4,5-tetrahydropyridin-6-yl]-2-(1,2,2-trimethyl-4-piperidyl)indazole (50 mg, 0.148 mmol) in MeOH (5 mL) was adjusted to pH=7 with Na₂CO₃, then NaBH₄ (10 mg, 0.264 mmol) was added at 0° C. slowly. The resulting mixture was stirred at 20° C. for 1 hour. The resulting mixture was quenched by addition of water (10 mL) and concentrated under reduced pressure to give a crude product which was purified by flash chromatography (Column: SepaFlash® Sphercial C18, 60 g, 40-60 m, 120A; MeCN/water (0.05 v % NH₃—H₂O) with MeCN from 0-60%, 35 mL/min, 254 nm) to afford 6-[(5S)-5-methyl-2-piperidyl]-2-(1,2,2-trimethyl-4-piperidyl)indazole (30 mg, 59.6% yield) as a yellow oil. LCMS (ESI) [M+H]⁺ m/z: calcd 314.3, found 314.3.

### Step 9: Synthesis of tert-butyl N-tert-butoxycarbonyl-N-[3-ethyl-5-[[2-oxo-2-[(5S)-5-methyl-2-[2-(1,2,2-trimethyl-4-piperidyl)indazol-6-yl]-1-piperidyl]acetyl]amino]-2-pyridyl]carbamate)

A mixture of 6-[(5S)-5-methyl-2-piperidyl]-2-(1,2,2-trimethyl-4-piperidyl)indazole (30.0 mg, 88.1 mol), 2-[[6-[bis(tert-butoxycarbonyl)amino]-5-ethyl-3-pyridyl]amino]-2-oxo-acetic acid (40 mg, 97.7 mol), DMF (2 mL), HATU (40 mg, 0.105 mmol) and DIPEA (0.05 mL, 0.287 mmol) was stirred at 20° C. for 2 hours. The resulting mixture was concentrated under reduced pressure to give tert-butyl N-tert-butoxycarbonyl-N-[3-ethyl-5-[[2-oxo-2-[(5S)-5-methyl-2-[2-(1,2,2-trimethyl-4-piperidyl)indazol-6-yl]-1-piperidyl]acetyl]amino]-2-pyridyl]carbamate (60 mg, crude) as a yellow oil.

### Step 10: Synthesis of N-(6-amino-5-ethyl-3-pyridyl)-2-oxo-2-[(5S)-5-methyl-2-[2-(1,2,2-trimethyl-4-piperidyl)indazol-6-yl]-1-piperidyl]acetamide

A mixture of tert-butyl N-tert-butoxycarbonyl-N-[3-ethyl-5-[[2-oxo-2-[(5S)-5-methyl-2-[2-(1,2,2-trimethyl-4-piperidyl)indazol-6-yl]-1-piperidyl]acetyl]amino]-2-pyridyl]carbamate (60 mg, 82.0 mol) and HCl/MeOH (4 M, 3 mL) was stirred at 20° C. for 12 hours. The resulting mixture was adjusted to pH=8 with NH₃—H₂O(12 N), then the mixture was filtered and concentrated under reduced pressure to afford crude product, which was purified by preparative HPLC (Instrument: Gilson GX-281 Liquid Handler, Gilson 322 Pump, Gilson 156 UV Detector; Column: 2_Phenomenex Gemini C18 75*40 mm*3 um; Mobile phase A: H₂O with NH₄HCO₃ (v %); Mobile phase B: MeCN; Gradient: B from 35% to 65% in 9.5 min, hold 100% B for 0 min; Flow Rate: 25 mL/min; Column Temperature: 30° C.; Wavelength: 220 nm, 254 nm) to afford N-(6-amino-5-ethyl-3-pyridyl)-2-oxo-2-[(5S)-5-methyl-2-[2-(1,2,2-trimethyl-4-piperidyl)indazol-6-yl]-1-piperidyl]acetamide (25 mg, 57.4% yield) as a yellow oil. LCMS (ESI) [M+H]⁺ m/z: calcd 532.3, found 532.3.

### Step 11: Synthesis of N-(6-amino-5-ethyl-3-pyridyl)-2-oxo-2-[(2R,5S)-5-methyl-2-[2-(1,2,2-trimethyl-4-piperidyl)indazol-6-yl]-1-piperidyl]acetamide (Compound 109)

N-(6-amino-5-ethyl-3-pyridyl)-2-oxo-2-[(5S)-5-methyl-2-[2-(1,2,2-trimethyl-4-piperidyl)indazol-6-yl]-1-piperidyl]acetamide (25.0 mg, 47.0 μmol) was purified by SFC-22 (Instrument: Berger, MULTIGR AM-II; Column: DAICEL CHIRALPAK AD (250 mm*30 mm*10 m); Mobile phase: supercritical Hexane-IPA (0.1% NH₃, MeOH v %)=50/50; Flow Rate:80 mL/min; Column Temperature: 38° C.; Nozzle Pressure: 100 bar; Nozzle Temperature: 60° C.; Evaporator Temperature: 20° C.; Trimmer Temperature: 25° C.; Wavelength:220 nm). The fraction was concentrated under reduced pressure and then lyophilized for overnight to afford N-(6-amino-5-ethyl-3-pyridyl)-2-oxo-2-[(2R,5S)-5-methyl-2-[2-(1,2,2-trimethyl-4-piperidyl)indazol-6-yl]-1-piperidyl]acetamide (11.4 mg, 45.6% yield) as a white solid. ¹H NMR (400 MHz, methanol-d₄) δ ppm 8.18-8.31 (m, 1H), 7.90-8.15 (m, 1H), 7.61-7.80 (m, 2H), 7.43-7.60 (m, 1H), 7.02-7.18 (m, 1H), 5.40-5.80 (m, 1H), 3.73 (br d, J=13.8 Hz, 1H), 3.37-3.51 (m, 1H), 2.72-2.90 (m, 2H), 2.39-2.57 (m, 2H), 2.33 (s, 5H), 2.21 (br s, 3H), 1.90-2.11 (m, 4H), 1.47 (br d, J=10.8 Hz, 1H), 1.26 (s, 5H), 1.17 (s, 4H), 1.14 (br d, J=6.8 Hz, 3H); LCMS (ESI) [M+H]⁺ m/z: calcd 532.3, found 532.3; HPLC: 100%@254 nm.

### Example 5. Compound 116 N-(6-amino-5-ethylpyridin-3-yl)-2-((2R,5S)-5-methyl-2-(2-(2-(pyrrolidin-1-yl)propyl)benzo[d]thiazol-5-yl)piperidin-1-yl)-2-oxoacetamide

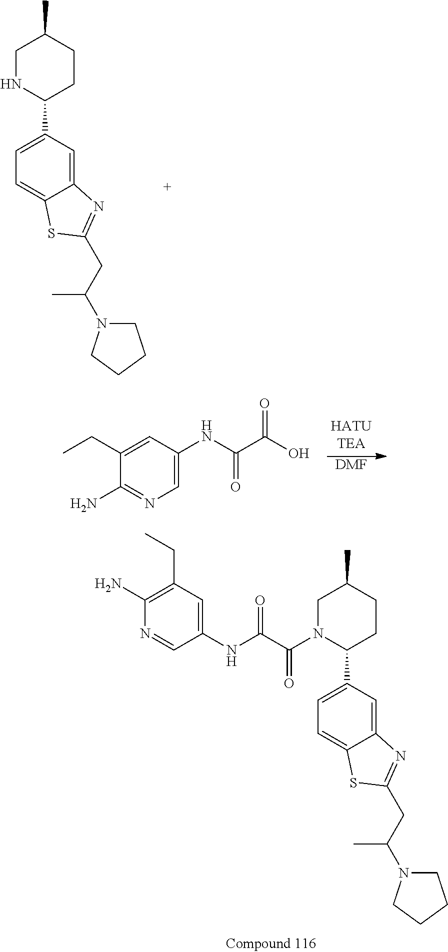

To a solution of 2-(2-pyrrolidin-1-ylpropyl)-5-[(2R,5S)-5-methyl-2-piperidyl]-1,3-benzothiazole (300.00 mg, 873.29 mol), 2-[(6-amino-5-ethyl-3-pyridyl)amino]-2-oxo-acetic acid (228.37 mg, 1.09 mmol) and TEA (353.47 mg, 3.49 mmol, 486.88 μL) in DMF (4 mL), HATU (448.27 mg, 1.18 mmol) was added. The resulting mixture was stirred at 25° C. for 3 hr. The reaction mixture was purified by HPLC (Device (Mobile Phase, Column): SYSTEM 25-25-55% 0-1-6 min H₂O/MeCN/0.1% NH₄OH, flow: 30 ml/min (loading pump 4 ml/min MeOH) target mass 534 column: XBridge BEH C18 5 μm 130 A) to give 72 mg (84%) crude product, which was purified by SFC (Chromatorex PEI (19*100, 5 mkm) flow 50 ml/min) from cis-impurities to afford N-(6-amino-5-ethyl-3-pyridyl)-2-oxo-2-[(2R,5S)-5-methyl-2-[2-(2-pyrrolidin-1-ylpropyl)-1,3-benzothiazol-5-yl]-1-piperidyl]acetamide (27 mg, 50.49 mol, 5.78% yield).

Compound 116: ¹H NMR (600 MHz, DMSO-d₆) δ (ppm) 1.07 (m, 9H), 1.36 (m, 1H), 1.70 (s, 5H), 1.87 (m, 1H), 2.13 (m, 1H), 2.35 (m, 4H), 2.61 (m, 4H), 2.95 (m, 1H), 3.13 (m, 2H), 4.04 (m, 1H), 5.64 (m, 3H), 7.36 (m, 1H), 7.47 (m, 1H), 7.86 (m, 1H), 8.03 (m, 2H), 10.55 (m, 1H).

LCMS(ESI): [M]⁺ m/z: calcd 534.2; found 535.2; Rt=2.024 min.

### Example 6. Compound 57 2-methoxy-5-(2-((2R,5S)-5-methyl-2-(2-(2-(pyrrolidin-1-yl)propyl)benzo[d]thiazol-5-yl)piperidin-1-yl)-2-oxoacetamido)nicotinamide

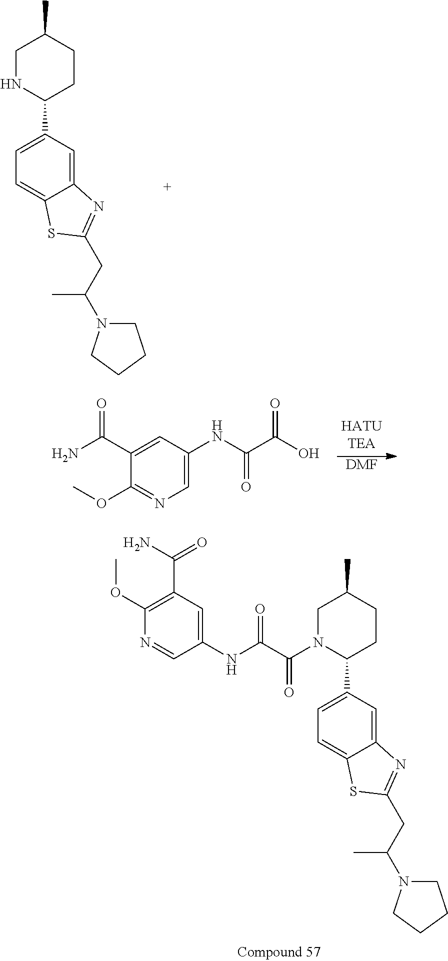

To a solution of 2-(2-pyrrolidin-1-ylpropyl)-5-[(2R,5S)-5-methyl-2-piperidyl]-1,3-benzothiazole (0.2 g, 582.19 mol), 2-[(5-carbamoyl-6-methoxy-3-pyridyl)amino]-2-oxo-acetic acid (167.10 mg, 698.63 mol) and TEA (235.65 mg, 2.33 mmol, 324.59 μL) in DMF (4 mL), HATU (287.78 mg, 756.85 mol) was added. The resulting mixture was stirred at 25° C. for 3 hr. The reaction mixture was purified by HPLC (Device (Mobile Phase, Column): SYSTEM 15-65% 0-5 min H₂O/MeCN/0.1% NH₄OH, flow: 30 ml/min (loading pump 4 ml/min MeCN) target mass 391.36 column: XBridge C18 100×19 mm, 5 um) to give 100 mg crude product, which was purified (Chiralcel OD-H (250*20 mm, 5 mkm); Hexane-MeOH-IPA, 50-25-25; 12 ml/min) from cis-impurities to afford 2-methoxy-5-[[2-oxo-2-[(2R,5S)-5-methyl-2-[2-(2-pyrrolidin-1-ylpropyl)-1,3-benzothiazol-5-yl]-1-piperidyl]acetyl]amino]pyridine-3-carboxamide (69 mg, 122.19 mol, 20.99% yield).

Compound 57: ¹H NMR (600 MHz, DMSO-d₆) δ (ppm) 1.04 (m, 3H), 1.18 (m, 2H), 1.37 (m, 1H), 1.79 (m, 6H), 2.25 (m, 2H), 2.86 (m, 5H), 3.41 (m, 3H), 3.94 (m, 5H), 5.50 (m, 1H), 7.40 (dd, 1H), 7.73 (m, 2H), 7.88 (m, 1H), 8.07 (m, 1H), 8.51 (m, 2H), 11.08 (m, 1H).

LCMS(ESI): [M]⁺ m/z: calcd 564.2; found 565.2; Rt=2.423 min.

### Example 7. Compound 62 and Compound 15 2-methoxy-5-(2-((2R,5S)-5-methyl-2-(2-(1-(pyrrolidin-1-yl)propan-2-yl)benzo[d]thiazol-5-yl)piperidin-1-yl)-2-oxoacetamido)nicotinamide

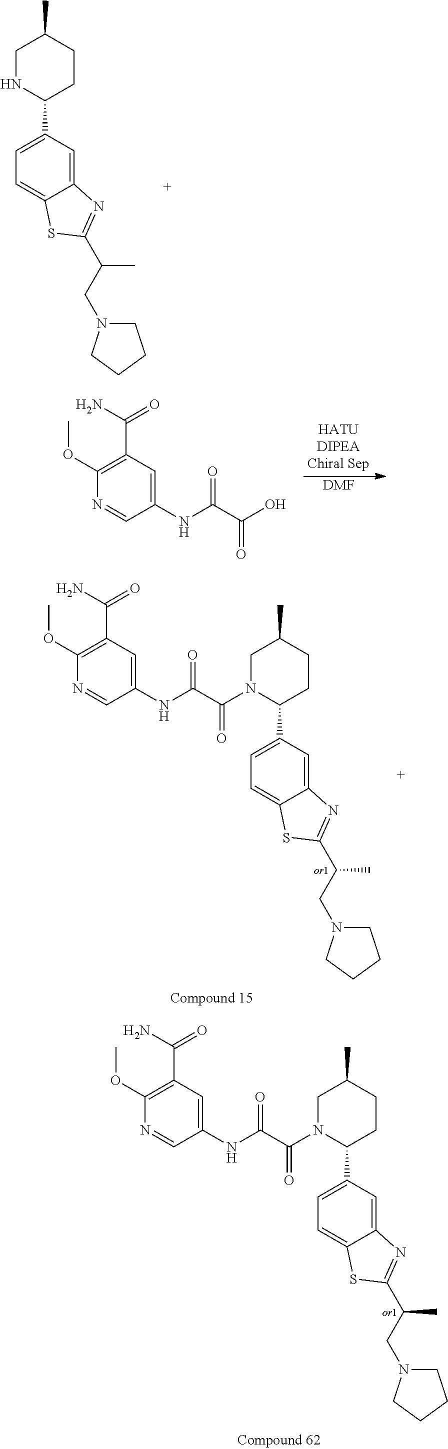

The synthesis of 5-((2R,5S)-5-methylpiperidin-2-yl)-2-(1-(pyrrolidin-1-yl)propan-2-yl)benzo[d]thiazole is given by 3EEE. 5-[(2R,5S)-5-Methyl-2-piperidyl]-2-[1-methyl-2-pyrrolidin-1-yl-ethyl]-1,3-benzothiazole (0.2 g, 582.19 mol), 2-[(5-carbamoyl-6-methoxy-3-pyridyl)amino]-2-oxo-acetic acid (139.25 mg, 582.19 μmol) and DIPEA (225.73 mg, 1.75 mmol, 304.22 μL) were mixed together at 25° C. and stirred for min. HATU (287.78 mg, 756.85 mol) was added thereto and the resulting reaction mixture was stirred at 25° C. for 12 hr. The obtained crude product was purified by reverse phase HPLC chromatography (Device (Mobile Phase, Column): SYSTEM 60-60-80% 0-1-6 min H₂O/MeOH/0.1% NH₄OH, flow: 30 ml/min (loading pump 4 ml/min MeOH) XBridge BEH C18 100×19 mm, 5 um) to afford crude product (103.8 mg).

The obtained crude product was purified by chiral column chromatography (Column: Chiralpak IC-III (250*21 mm, 5 mkm), IPA-MeOH, 50-50, 10 ml/min) to obtain 2-methoxy-5-[[2-oxo-2-[(2R,5S)-5-methyl-2-[2-[(1S)-1-methyl-2-pyrrolidin-1-yl-ethyl]-1,3-benzothiazol-5-yl]-1-piperidyl]acetyl]amino]pyridine-3-carboxamide (0.03996 g, 70.76 mol, 12.15% yield) (RT=51.09 min) and 2-methoxy-5-[[2-oxo-2-[(2R,5S)-5-methyl-2-[2-[(1R)-1-methyl-2-pyrrolidin-1-yl-ethyl]-1,3-benzothiazol-5-yl]-1-piperidyl]acetyl]amino]pyridine-3-carboxamide (0.03908 g, 69.21 mol, 11.89% yield) (RT=98.87 min).

Rel Time for Compound 62 in analytical conditions (column: IC, IPA-MeOH, 50-50, 0.6 ml/min as mobile phase) 41.14 min and for Compound 15 80.21 min.

### Compound 62: Retention Time: 41.14 Min

1H NMR (DMSO-d6, 500 MHz): δ (ppm) 1.04 (m, 3H), 1.37 (m, 4H), 1.69 (m, 5H), 1.88 (m, 1H), 2.14 (m, 1H), 2.31 (m, 1H), 2.58 (m, 4H), 2.63 (m, 1H), 2.83 (m, 1H), 3.42 (m, 2H), 3.94 (m, 4H), 5.49 (m, 1H), 7.37 (m, 1H), 7.72 (m, 2H), 7.87 (m, 1H), 8.03 (m, 1H), 8.51 (m, 2H), 11.07 (m, 1H)

LCMS(ESI): [M]⁺ m/z: calcd 564.2; found 565.2; Rt=2.315 min.

### Compound 15: Retention Time: 80.21 Min

1H NMR (DMSO-d6, 500 MHz): δ (ppm) 1.04 (m, 3H), 1.37 (m, 4H), 1.66 (m, 5H), 1.88 (m, 1H), 2.19 (m, 3H), 2.58 (m, 6H), 2.83 (m, 1H), 3.94 (m, 4H), 5.49 (m, 1H), 7.37 (m, 1H), 7.72 (m, 2H), 7.88 (m, 1H), 8.03 (m, 1H), 8.49 (m, 2H), 11.07 (m, 1H)

LCMS(ESI): [M]⁺ m/z: calcd 564.2; found 565.2; Rt=2.311 min.

### Example 8. Compound 14 and Compound 11 5-(2-((2R,5S)-2-(2-(1-(dimethylamino)propan-2-yl)benzo[d]thiazol-5-yl)-5-methylpiperidin-1-yl)-2-oxoacetamido)-2-methoxynicotinamide

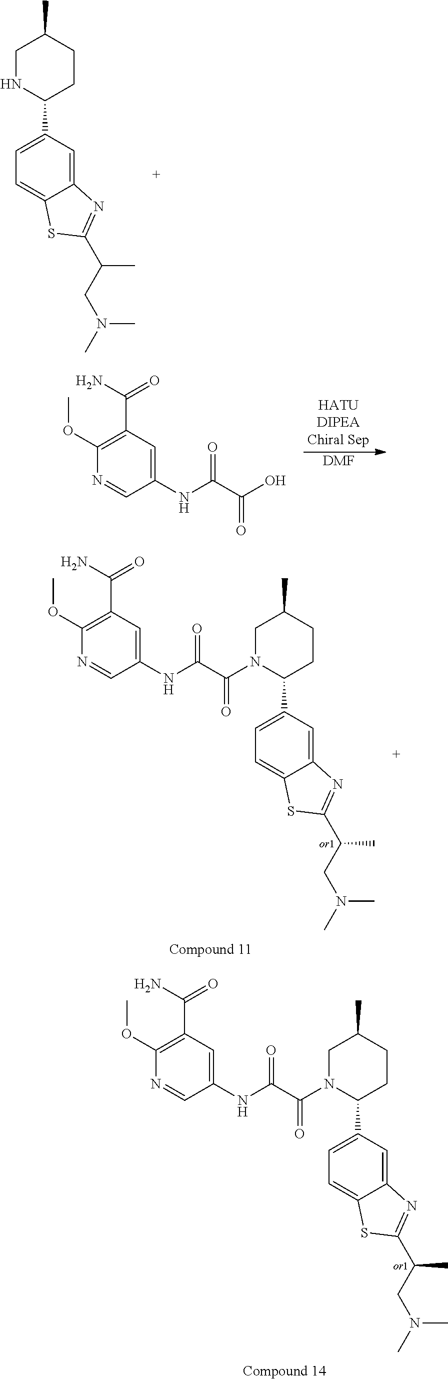

The synthesis of N,N-dimethyl-2-(5-((2R,5S)-5-methylpiperidin-2-yl)benzo[d]thiazol-2-yl)propan-1-amine is given by 3PPP.

To a stirred solution of N,N-dimethyl-2-[5-[(2R,5S)-5-methyl-2-piperidyl]-1,3-benzothiazol-2-yl]propan-1-amine (0.1 g, 314.97 mol), 2-[(5-carbamoyl-6-methoxy-3-pyridyl)amino]-2-oxo-acetic acid (0.09 g, 376.28 mol) and DIPEA (148.40 mg, 1.15 mmol, 0.2 mL) in DMF (8 mL) was added HATU (0.15 g, 394.50 mol) at 5-10° C. The resulting reaction mixture was stirred at 25° C. for 24 hr. Upon completion, the reaction mixture was concentrated under reduced pressure to obtain crude product. The obtained crude product was purified by reverse phase HPLC chromatography (Device (Mobile Phase, Column): SYSTEM 40-40-85% 0-1-6 min H₂O/MeOH/0.1% NH₄OH, flow: 30 ml/min (loading pump 4 ml/min MeOH) target mass 538 column: YMC Triart C18 100×20 mm, 5 um) to afford crude product (75 mg).

The obtained crude product was purified by chiral column chromatography (Column: Chiralpak IC-III (250*20 mm, 5 mkm), IPA-MeOH, 50-50, 10 ml/min) to obtain 2-methoxy-5-[[2-oxo-2-[(2R,5S)-5-methyl-2-[2-[(¹R)-2-(dimethylamino)-1-methyl-ethyl]-1,3-benzothiazol-5-yl]-1-piperidyl]acetyl]amino]pyridine-3-carboxamide (30 mg, 55.69 μmol, 17.68% yield) and 2-methoxy-5-[[2-oxo-2-[(2R,5S)-5-methyl-2-[2-[(¹S)-2-(dimethylamino)-1-methyl-ethyl]-1,3-benzothiazol-5-yl]-1-piperidyl]acetyl]amino]pyridine-3-carboxamide (30 mg, 55.69 μmol, 17.68% yield).

Rel Time for Compound 14 in analytical conditions (column: IC, IPA-MeOH, 50-50, 0.6 ml/min as mobile phase) 70.38 min and for Compound 11 34.62 min.

### Compound 14: Retention Time: 70.38 Min

1H NMR (600 MHz, dmso) δ 1.00-1.09 (m, 3H), 1.31-1.43 (m, 4H), 1.68-1.77 (m, 1H), 1.84-1.96 (m, 1H), 2.05-2.15 (m, 1H), 2.18 (s, 6H), 2.26-2.35 (m, 1H), 2.60-2.86 (m, 2H), 3.35-3.47 (m, 2H), 3.49-4.05 (m, 4H), 5.26-5.74 (m, 1H), 7.29-7.43 (m, 1H), 7.64-7.79 (m, 2H), 7.83-7.92 (m, 1H), 7.99-8.07 (m, 1H), 8.39-8.49 (m, 1H), 8.49-8.61 (m, 1H), 11.02-11.14 (m, 1H).

LCMS(ESI): [M]⁺ m/z: calcd 538.2; found 539.2; Rt=1.040 min.

### Compound 11: Retention Time: 34.62 Min

1H NMR (600 MHz, dmso) δ 1.00-1.08 (m, 3H), 1.31-1.42 (m, 4H), 1.65-1.77 (m, 1H), 1.82-1.94 (m, 1H), 2.04-2.14 (m, 1H), 2.18 (s, 6H), 2.27-2.35 (m, 1H), 2.41-2.46 (m, 1H), 2.60-2.88 (m, 2H), 3.42-3.47 (m, 1H), 3.50-4.05 (m, 4H), 5.28-5.74 (m, 1H), 7.31-7.44 (m, 1H), 7.65-7.79 (m, 2H), 7.85-7.92 (m, 1H), 8.01-8.08 (m, 1H), 8.39-8.49 (m, 1H), 8.49-8.61 (m, 1H), 10.97-11.15 (m, 1H).

LCMS(ESI): [M]⁺ m/z: calcd 538.2; found 539.2; Rt=1.037 min.

### Example 9. Compound 80 N-(6-amino-5-ethyl-3-pyridyl)-2-oxo-2-[(2R,5S)-5-methyl-2-[2-[rel-(3R)-1-methyl-3-piperidyl]indazol-6-yl]-1-piperidyl]acetamide

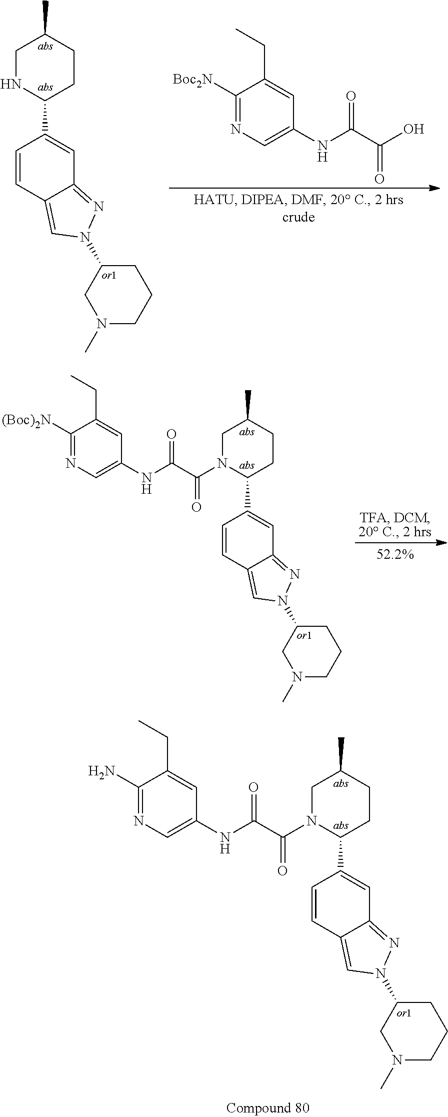

### Step 1: Synthesis of tert-butyl N-tert-butoxycarbonyl-N-[3-ethyl-5-[[2-oxo-2-[(2R,5S)-5-methyl-2-[2-[rel-(3R)-1-methyl-3-piperidyl]indazol-6-yl]-1-piperidyl]acetyl]amino]-2-pyridyl]carbamate

To a mixture of 6-[(2R,5S)-5-methyl-2-piperidyl]-2-[rel-(3R)-1-methyl-3-piperidyl]indazole (40 mg, 0.128 mmol) and 2-[[6-[bis(tert-butoxycarbonyl)amino]-5-ethyl-3-pyridyl]amino]-2-oxo-acetic acid (60.00 mg, 0.147 mmol) in DMF (3 mL) were added HATU (58 mg, 0.153 mmol) and DIPEA (70.0 μL, 0.402 mmol). The resulting mixture was stirred at 20° C. for 2 hours. The resulting mixture was concentrated under reduced pressure to give tert-butyl N-tert-butoxycarbonyl-N-[3-ethyl-5-[[2-oxo-2-[(2R,5S)-5-methyl-2-[2-[rel-(3R)-1-methyl-3-piperidyl]indazol-6-yl]-1-piperidyl]acetyl]amino]-2-pyridyl]carbamate (90 mg, crude) as yellow oil.

### Step 2: Synthesis of N-(6-amino-5-ethyl-3-pyridyl)-2-oxo-2-[(2R,5S)-5-methyl-2-[2-[rel-(3R)-1-methyl-3-piperidyl]indazol-6-yl]-1-piperidyl]acetamide (Compound 80)

A mixture of tert-butyl N-tert-butoxycarbonyl-N-[3-ethyl-5-[[2-oxo-2-[(2R,5S)-5-methyl-2-[2-[rel-(3R)-1-methyl-3-piperidyl]indazol-6-yl]-1-piperidyl]acetyl]amino]-2-pyridyl]carbamate (90 mg, 0.128 mmol), DCM (2 mL) and TFA (2 mL, 26.0 mmol) was stirred at 20° C. for 2 hours. The resulting mixture was adjusted to pH=8 with NH₃—H₂O(12 N) solution. The mixture was concentrated under reduced pressure to give a crude product, which was purified by preparative HPLC (Instrument: Gilson GX-281 Liquid Handler, Gilson 322 Pump, Gilson 156 UV Detector; Column: 2_Phenomenex Gemini Cis 75*40 mm*3 um; Mobile phase A: H₂O with 0.05% NH₃—H₂O (v %); Mobile phase B: MeCN; Gradient: B from 32% to 62% in 9.5 min, hold 100% B for 0 min; Flow Rate: 25 mL/min; Column Temperature: 30° C.; Wavelength: 220 nm, 254 nm).

Compound 80: N-(6-amino-5-ethyl-3-pyridyl)-2-oxo-2-[(2R,5S)-5-methyl-2-[2-[rel-(3R)-1-methyl-3-piperidyl]indazol-6-yl]-1-piperidyl]acetamide (33.6 mg, single unknown enantiomer with trans relative chemistry, white solid). ¹H NMR (400 MHz, methanol-d₄) δ ppm 8.29 (br s, 1H), 8.11 (br s, 1H), 7.71 (br d, J=8.8 Hz, 1H), 7.41-7.67 (m, 2H), 7.02-7.17 (m, 1H), 5.35-5.80 (m, 1H), 3.67-4.10 (m, 1H), 3.41 (br d, J=13.6 Hz, 1H), 3.09-3.24 (m, 1H), 2.88 (br d, J=11.3 Hz, 1H), 2.46-2.61 (m, 2H), 2.12-2.44 (m, 7H), 1.69-2.11 (m, 6H), 1.44 (br d, J=10.0 Hz, 1H), 1.24 (brt, J=7.0 Hz, 2H), 1.09-1.20 (m, 4H); LCMS (ESI) [M+H]⁺ m/z: calcd 504.3, found 504.3; HPLC: 100%@254 nm; SFC: 99.7% ee.

### Example 10. Compound 88 2-methoxy-5-(2-((2R,5S)-5-methyl-2-(2-(1-methylpiperidin-4-yl)quinolin-7-yl)piperidin-1-yl)-2-oxoacetamido)nicotinamide

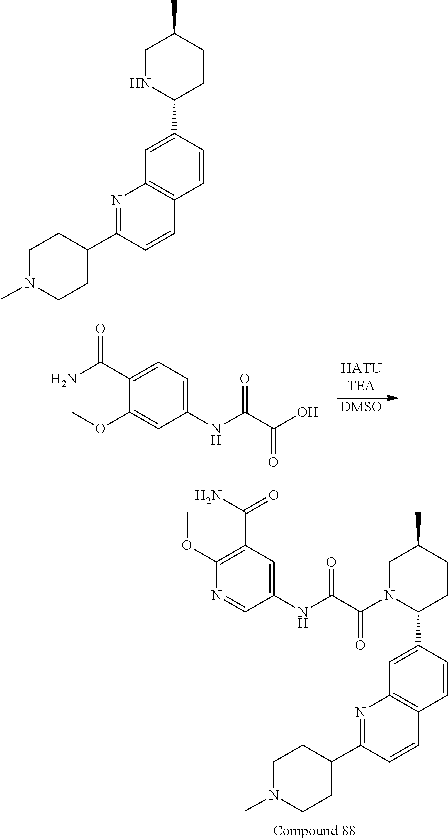

The synthesis of 2-(1-methyl-4-piperidyl)-7-[(2R,5S)-5-methyl-2-piperidyl]quinoline is given by 3RRR.

Crude product 2-(1-methyl-4-piperidyl)-7-[(2R,5S)-5-methyl-2-piperidyl]quinoline (100.00 mg, 216.40 mol) from previous stage was mixed with TEA (109.49 mg, 1.08 mmol, 150.81 μL) in DMSO (2 mL), next 2-[(5-carbamoyl-6-methoxy-3-pyridyl)amino]-2-oxo-acetic acid (62.11 mg, 259.68 μmol) and HATU (98.74 mg, 259.68 mol) were added and stirred overnight. RM was treated with water and desired product was filtered, washed with water and dissolved in MeOH, then subjected to HPLC (2-10 min 30% water MeCN+FA 30/min; loading pump 4 ml/min MeCN column SunFire 19*100 mm). 2-Methoxy-5-[[2-oxo-2-[(2R,5S)-5-methyl-2-[2-(1-methyl-4-piperidyl)-7-quinolyl]-1-piperidyl]acetyl]amino]pyridine-3-carboxamide (28.2 mg, 47.74 mol, 22.06% yield, HCOOH) was obtained.

Compound 88: ¹H NMR (600 MHz, DMSO-d₆) δ (ppm) 1.05 (dd, 3H), 1.39 (t, 1H), 1.72 (d, 1H), 1.90 (d, 5H), 2.13 (d, 2H), 2.27 (d, 3H), 2.34 (s, 1H), 2.84 (d, 2H), 2.96 (s, 2H), 3.94 (d, 4H), 5.57 (d, 1H), 7.49 (m, 2H), 7.78 (m, 3H), 7.94 (dd, 1H), 8.24 (m, 2H), 8.52 (m, 2H), 11.10 (d, 1H).

LCMS(ESI): [M]⁺ m/z: calcd 544.2; found 545.2; Rt=2.266 min.

### Example 11. Compound 91 N-(6-amino-5-ethylpyridin-3-yl)-2-((2R,5S)-2-(2-(7,7-difluoro-9-methyl-9-azabicyclo[3.3.1]nonan-3-yl)benzo[d]thiazol-5-yl)-5-methylpiperidin-1-yl)-2-oxoacetamide

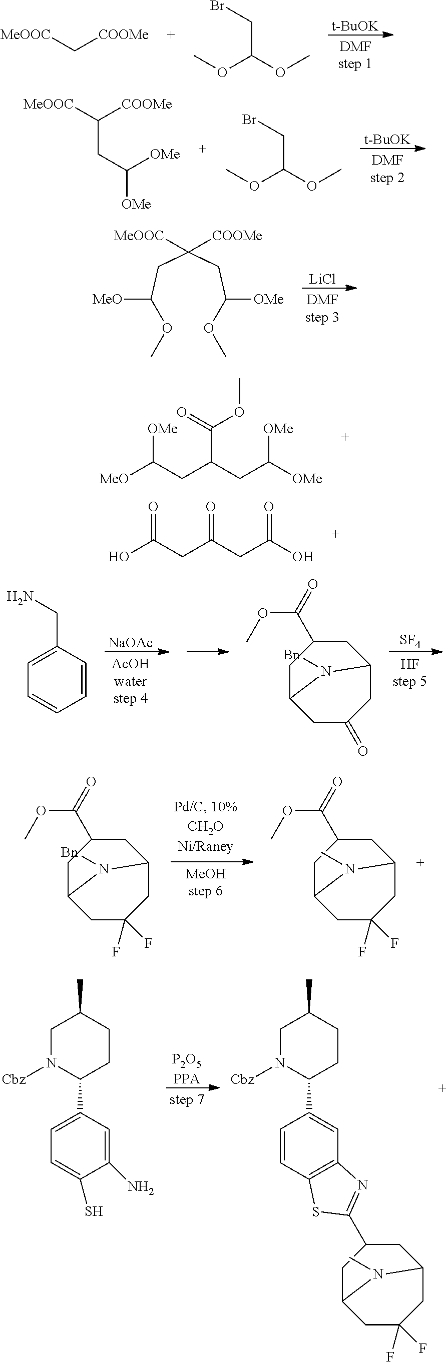

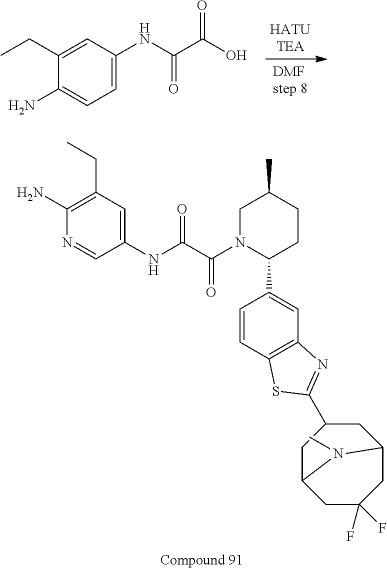

### Step 1: Synthesis of dimethyl 2-(2,2-dimethoxyethyl)malonate

Dimethyl propanedioate (13.7 g, 103.70 mmol, 11.85 mL) was suspended in DMF (150 mL) and potassium tert-butoxide (12.22 g, 108.88 mmol) was added thereto. Once the addition has finished the temperature is raised to 60° C. and 2-bromo-1,1-dimethoxy-ethane (17.53 g, 103.70 mmol, 12.26 mL) was added thereto. The temperature is raised to 90° C. and kept overnight. The reaction mixture was poured into H₂O (500 ml) and extracted with EtOAc (3×150 ml), the organic layer was washed with brine, dried over Na₂SO₄, concentrated to afford dimethyl 2-(2,2-dimethoxyethyl)propanedioate (17.8 g, crude).

¹H NMR (400 MHz, CDCl₃) δ (ppm) 2.18 (m, 2H), 3.26 (s, 6H), 3.55 (m, 1H), 3.69 (s, 6H), 4.36 (m, 1H).

### Step 2: Synthesis of dimethyl 2,2-bis(2,2-dimethoxyethyl)malonate

Dimethyl 2-(2,2-dimethoxyethyl)propanedioate (16.4 g, 74.47 mmol) was suspended in DMF (130.32 mL) and potassium tert-butoxide (8.77 g, 78.19 mmol) was added thereto. Once the addition has finished the temperature is raised to 60° C. and 2-bromo-1,1-dimethoxy-ethane (12.59 g, 74.47 mmol, 8.80 mL) was added thereto. The temperature is raised to 120° C. and kept overnight. Then reaction mixture was cooled to rt and poured into H₂O (200 ml) and extracted with EtOAc (3×100 ml), the organic layer was washed with brine, dried over Na₂SO₄, concentrated to afford dimethyl 2,2-bis(2,2-dimethoxyethyl)propanedioate (17.75 g, crude).

¹H NMR (500 MHz, CDCl₃) δ (ppm) 2.25 (m, 4H), 3.28 (s, 12H), 3.66 (s, 6H), 4.38 (m, 2H).

### Step 3: Synthesis of methyl 2-(2,2-dimethoxyethyl)-4,4-dimethoxybutanoate

Dimethyl 2,2-bis(2,2-dimethoxyethyl)propanedioate (17.75 g, 57.57 mmol) was dissolved in DMF (150 mL) and lithium chloride (salt) (4.88 g, 115.14 mmol) was added thereto. The temperature is raised to 150° C. and kept overnight. Then reaction mixture was cooled to rt and poured into H₂O (200 ml) and extracted with EtOAc (3×150 ml), the organic layer was washed with brine, dried over Na₂SO₄, concentrated to afford methyl 2-(2,2-dimethoxyethyl)-4,4-dimethoxy-butanoate (11.65 g, crude).

¹H NMR (400 MHz, CDCl₃) δ (ppm) 1.74 (m, 2H), 1.96 (m, 2H), 2.62 (m, 1H), 3.31 (s, 12H), 3.69 (s, 3H), 4.37 (m, 2H).

### Step 4: Synthesis of methyl 9-benzyl-7-oxo-9-azabicyclo[3.3.1]nonane-3-carboxylate

Methyl 2-(2,2-dimethoxyethyl)-4,4-dimethoxy-butanoate (5.5 g, 21.97 mmol) in a mixture acetic acid (10 mL) and water (40 mL) was heated at reflux for 1 hr then cooled to rt. Phenylmethanamine (2.35 g, 16.40 mmol, HCl) and 3-oxopentanedioic acid (3.21 g, 21.97 mmol), sodium acetate, anhydrous (8.11 g, 98.89 mmol, 5.31 mL) and water (50 mL) were added and the reaction heated at 50° C. for 16 hr. The reaction was cooled to rt and acidified to pH=6.8 using K₂CO₃ 50% and the mixture is extracted with EtOAc (3×30) and the combined organic fractions washed with brine, dried over Na₂SO₄, concentrated to afford methyl 9-benzyl-3-oxo-9-azabicyclo[3.3.1]nonane-7-carboxylate (3.5 g, 12.18 mmol, 55.43% yield). The residue was purified by column chromatography (Interchim; 120 g SiO₂, HEX-EtOAc from 0˜100%, flow rate=80 mL/min, cv=10.1).

LCMS(ESI): [M]⁺ m/z: calcd 287.2; found 288.2; Rt=0.738 min.

### Step 5: Synthesis of methyl 9-benzyl-7,7-difluoro-9-azabicyclo[3.3.1]nonane-3-carboxylate

Methyl 9-benzyl-3-oxo-9-azabicyclo[3.3.1]nonane-7-carboxylate (2.5 g, 8.70 mmol) and HF (5 mL) were placed in a Hastelloy autoclave. It was cooled to −196° C., evacuated and approximately sulfur tetrafluoride (2.82 g, 26.10 mmol) were condensed. The autoclave was kept at 20° C. for 10 hr, the gaseous products were released and the reaction mixture was poured onto K₂CO₃/H₂O. The product was extracted with EtOAc, the combined extracts were washed with water, dried over Na₂SO₄ and evaporated to afford methyl 9-benzyl-3,3-difluoro-9-azabicyclo[3.3.1]nonane-7-carboxylate (2.4 g, crude).

LCMS(ESI): [M]⁺ m/z: calcd 309.2; found 310.2; Rt=0.838 min.

### Step 6: Synthesis of methyl 7,7-difluoro-9-methyl-9-azabicyclo[3.3.1]nonane-3-carboxylate

For neutralize sulfur impurities from the previous stage Raney Nickel, active catalyst (4 g, 8.08 mmol) was added to a stirred solution of methyl 9-benzyl-7,7-difluoro-9-azabicyclo[3.3.1]nonane-3-carboxylate (2.5 g, 8.08 mmol) in MeOH 40 mL. The resultant suspension was stirred 18 hr at rt. After completion the reaction mixture was filtered through small amount silica gel and evaporated under reduced pressure to give 1.2 g yellow oil. Then oil was dissolved in MeOH (25.00 mL) palladium, 10% on carbon, Type 487, dry (86.00 mg, 808.15 μmol) and Formaldehyde, 37% w/w aq. soln., stab. with 7-8% MeOH (2.43 g, 80.81 mmol, 2.24 mL) was added to a stirred solution. The resultant suspension was stirred under H₂ (1 atm) 200 hr at 20° C. Conversion was monitored by aliquots every day. After full conversion resultant suspension was filtered and the filtrate was concentrated in vacuum to afford methyl 7,7-difluoro-9-methyl-9-azabicyclo[3.3.1]nonane-3-carboxylate (0.625 g, crude).

GCMS: [M]: calcd 233.2; found 233.2; Rt=7.886 min.

### Step 7: Synthesis of 2-(7,7-difluoro-9-methyl-9-azabicyclo[3.3.1]nonan-3-yl)-5-((2R,5S)-5-methylpiperidin-2-yl)benzo[d]thiazole

Phosphorus (V) pentoxide (2 g, 14.09 mmol, 869.57 μL) was added portion wise to the stirred solution of methyl 7,7-difluoro-9-methyl-9-azabicyclo[3.3.1]nonane-3-carboxylate (56 mg, 240.08 μmol) in phosphoric acid (1.70 g, 14.74 mmol, 1 mL, 85% purity). After solution became homogenous, benzyl (2R,5S)-2-(3-amino-4-sulfanyl-phenyl)-5-methyl-piperidine-1-carboxylate (85.58 mg, 240.08 mol) was added thereto. Resulting mixture was stirred at 120° C. for 18 hr. Then, it was cooled, diluted with water/crushed ice and basified to pH≈10 with 20% aq. NaOH solution. Resulting slurry was extracted with DCM (2×15 ml). Organic layer was separated, dried over K₂CO₃ and concentrated under reduced pressure, affording 2-(7,7-difluoro-9-methyl-9-azabicyclo[3.3.1]nonan-3-yl)-5-[(2R,5S)-5-methyl-2-piperidyl]-1,3-benzothiazole (90 mg, 221.92 μmol, 92.44% yield).

LCMS(ESI): [M]⁺ m/z: calcd 405.2; found 406.2; Rt=0.612 min.

### Step 8: Synthesis of N-(6-amino-5-ethylpyridin-3-yl)-2-((2R,5S)-2-(2-(7,7-difluoro-9-methyl-9-azabicyclo[3.3.1]nonan-3-yl)benzo[d]thiazol-5-yl)-5-methylpiperidin-1-yl)-2-oxoacetamide (Compound 91)

To a stirred mixture of 2-(7,7-difluoro-9-methyl-9-azabicyclo[3.3.1]nonan-3-yl)-5-[(2R,5S)-5-methyl-2-piperidyl]-1,3-benzothiazole (90.00 mg, 221.92 mol), 2-[(6-amino-5-ethyl-3-pyridyl)amino]-2-oxo-acetic acid (51.07 mg, 244.12 mol) and TEA (89.83 mg, 887.69 μmol, 123.73 μL) in DMF (2 mL) was added HATU (97.04 mg, 255.21 μmol). The resulting reaction mixture was stirred at 30° C. for 4 hr. Then, it was subjected to HPLC (1-st run: 40-65% 0-5 min H₂O/MeCN/0.1% NH₄OH, flow: 30 ml/min; column: XBridge C18 100×19 mm, 5 um. 2-nd run:10-60% 0-5 min H₂O/MeCN/0.1% NH₄OH, flow: 30 ml/min; column: XBridge C18 100×19 mm, 5 um. 3-rd run:5-30% 0-5 min H₂O/MeCN/0.1% FA, flow: 30 ml/min; column: Chromatorex 18 SMB100-5T 100×19 mm 5 um), affording N-(6-amino-5-ethyl-3-pyridyl)-2-oxo-2-[(2R,5S)-2-[2-(7,7-difluoro-9-methyl-9-azabicyclo[3.3.1]nonan-3-yl)-1,3-benzothiazol-5-yl]-5-methyl-1-piperidyl]acetamide (12.8 mg, 19.91 mol, 8.97% yield, HCOOH).

Compound 91: ¹H NMR (600 MHz, DMSO-d₆) δ (ppm) 0.99-1.05 (m, 3H), 1.06-1.17 (m, 3H), 1.26-1.46 (m, 1H), 1.63-1.77 (m, 1H), 1.79-1.92 (m, 1H), 1.91-2.02 (m, 2H), 2.05-2.23 (m, 5H), 2.26-2.42 (m, 5H), 2.42-2.45 (m, 3H), 2.70-3.09 (m, 1H), 3.18-3.23 (m, 2H), 3.47-3.52 (m, 0.7H), 3.78-3.89 (m, 1H), 4.01-4.07 (m, 0.3H), 5.24-5.61 (m, 1H), 5.62-5.77 (m, 2H), 7.31-7.42 (m, 1H), 7.42-7.57 (m, 1H), 7.83-7.93 (m, 1H), 7.97-8.17 (m, 2H), 10.43-10.65 (m, 1H).

LCMS(ESI): [M]⁺ m/z: calcd 596.2; found 597.2; Rt=1.323 min.

### Example 12. Compound 69 and Compound 121 N-(6-amino-5-ethylpyridin-3-yl)-2-((2R,5S)-5-methyl-2-(3-((1-methylpyrrolidin-2-yl)methoxy)phenyl)piperidin-1-yl)-2-oxoacetamide

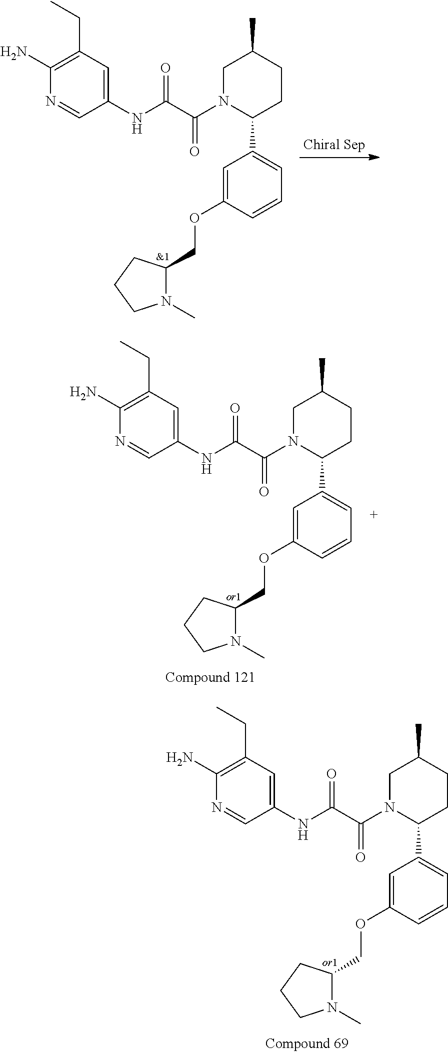

Racemic N-(6-amino-5-ethyl-3-pyridyl)-2-oxo-2-[(2R,5S)-5-methyl-2-[3-[[rac-(25)-1-methylpyrrolidin-2-yl]methoxy]phenyl]-1-piperidyl]acetamide (47.1 mg, 98.20 mol) was chiral separated (Column: Chiralpak IA (250*20 mm, 5 mkm); Mobile phase: IPA-MeOH, 50-50. Flow Rate: 12 mL/min) to obtain N-(6-amino-5-ethyl-3-pyridyl)-2-oxo-2-[(2R,5S)-5-methyl-2-[3-[[rel-(2R)-1-methylpyrrolidin-2-yl]methoxy]phenyl]-1-piperidyl]acetamide (16.55 mg, 34.51 mol, 35.14% yield) (RT=48.66 min) and N-(6-amino-5-ethyl-3-pyridyl)-2-oxo-2-[(2R,5S)-5-methyl-2-[3-[[rel-(2S)-1-methylpyrrolidin-2-yl]methoxy]phenyl]-1-piperidyl]acetamide (22.97 mg, 47.89 mol, 48.77% yield) (RT=15.51 min).

Rel Time for Compound 69 in analytical conditions (column: IA, IPA-MeOH, 50-50, 0.6 ml/min as mobile phase) 37.51 min and for Compound 121 12.99 min.

### Compound 69: Retention Time: 37.51 Min

¹H NMR (600 MHz, DMSO-d₆) δ (ppm) 0.99-1.03 (m, 3H), 1.07-1.13 (m, 3H), 1.27-1.36 (m, 1H), 1.55-1.69 (m, 4H), 1.79-2.05 (m, 3H), 2.14-2.24 (m, 2H), 2.32-2.34 (m, 3H), 2.39-2.41 (m, 1H), 2.75-3.26 (m, 3H), 3.40-4.02 (m, 4H), 5.08-5.57 (m, 1H), 5.57-5.67 (m, 2H), 6.75-6.92 (m, 3H), 7.24-7.32 (m, 1H), 7.41-7.54 (m, 1H), 7.97-8.08 (m, 1H), 10.47-10.55 (m, 1H).

LCMS(ESI): [M]⁺ m/z: calcd 479.2; found 480.2; Rt=0.873 min.

### Compound 121: Retention Time: 12.99 Min

¹H NMR (600 MHz, DMSO-d₆) δ (ppm) 0.95-1.03 (m, 3H), 1.06-1.14 (m, 3H), 1.25-1.37 (m, 1H), 1.54-1.69 (m, 4H), 1.77-2.25 (m, 6H), 2.28-2.34 (m, 3H), 2.38-2.42 (m, 1H), 2.73-3.27 (m, 3H), 3.44-4.04 (m, 3H), 5.08-5.56 (m, 1H), 5.56-5.69 (m, 2H), 6.78-6.93 (m, 3H), 7.24-7.30 (m, 1H), 7.41-7.51 (m, 1H), 7.98-8.10 (m, 1H), 10.47-10.59 (m, 1H).

LCMS(ESI): [M]⁺ m/z: calcd 479.2; found 480.2; Rt=0.872 min.

### Example 13. Compound 12 2-methoxy-5-[[2-oxo-2-[(2R,5S)-5-methyl-2-[2-[rel-(3R,4S)-1,3-dimethyl-4-piperidyl]-1,3-benzothiazol-5-yl]-1-piperidyl]acetyl]amino]pyridine-3-carboxamide (Compound ent-12) and 2-methoxy-5-[[2-oxo-2-[(2R,5S)-5-methyl-2-[2-[rel-(3R,4R)-1,3-dimethyl-4-piperidyl]-1,3-benzothiazol-5-yl]-1-piperidyl]acetyl]amino]pyridine-3-carboxamide

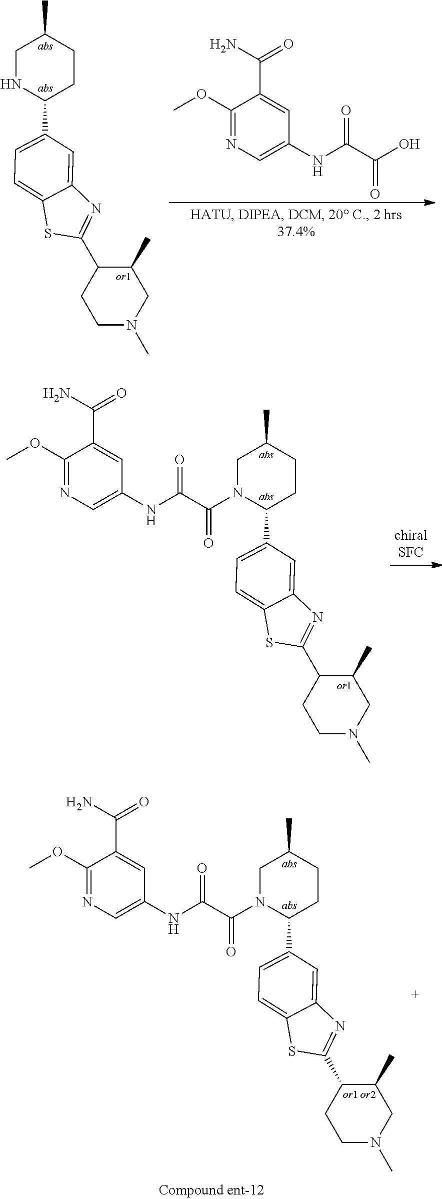

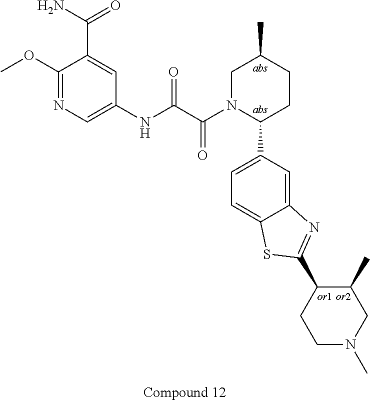

### Step 1: Synthesis of 2-methoxy-5-[[2-oxo-2-[(2R,5S)-5-methyl-2-[2-[rac-(3R)-1,3-dimethyl-4-piperidyl]-1,3-benzothiazol-5-yl]-1-piperidyl]acetyl]amino]pyridine-3-carboxamide

A mixture of 2-[(5-carbamoyl-6-methoxy-3-pyridyl)amino]-2-oxo-acetic acid (85 mg, 355.37 mol),2-[rac-(3R)-1,3-dimethyl-4-piperidyl]-5-[(2R,5S)-5-methyl-2-piperidyl]-1,3-benzothiazole (80 mg, 0.233 mmol), HATU (130 mg, 0.342 mmol), DIPEA (130 μL, 0.746 mmol) in DMF (4 mL) was stirred at 20° C. for 2 hours. The residue was purified by preparative HPLC (Instrument: Gilson GX-281 Liquid Handler, Gilson 322 Pump, Gilson 156 UV Detector; Column: Phenomenex C18 80×40 mm×3 m; Mobile phase A: H₂O with 0.05% NH₃—H₂O (v %); Mobile phase B: MeCN; Gradient: B from 40% to 70% in 9.5 min, hold 100% B for 0 min; Flow Rate: 30 mL/min; Column Temperature: 30° C.; Wavelength: 220 nm, 254 nm) to afford 2-methoxy-5-[[2-oxo-2-[(2R,5S)-5-methyl-2-[2-[rac-(3R)-1,3-dimethyl-4-piperidyl]-1,3-benzothiazol-5-yl]-1-piperidyl]acetyl]amino]pyridine-3-carboxamide (50 mg, 37.4% yield) as a white solid. LCMS (ESI) [M+H]⁺ m/z: calcd 565.3, found 565.3.

### Step 2: Synthesis of 2-methoxy-5-[[2-oxo-2-[(2R,5S)-5-methyl-2-[2-[rel-(3R,4S)-1,3-dimethyl-4-piperidyl]-1,3-benzothiazol-5-yl]-1-piperidyl]acetyl]amino]pyridine-3-carboxamide (Compound ent-12) and 2-methoxy-5-[[2-oxo-2-[(2R,5S)-5-methyl-2-[2-[rel-(3R,4R)-1,3-dimethyl-4-piperidyl]-1,3-benzothiazol-5-yl]-1-piperidyl]acetyl]amino]pyridine-3-carboxamide (Compound 12)

2-methoxy-5-[[2-oxo-2-[(2R,5S)-5-methyl-2-[2-[rac-(3R)-1,3-dimethyl-4-piperidyl]-1,3-benzothiazol-5-yl]-1-piperidyl]acetyl]amino]pyridine-3-carboxamide (50 mg, 0.089 mmol) was purified by chiral SFC (Instrument: Berger, MULTIGR AM-II; Column: Chiralpak OD 250×30 mm I.D. 10 m; Mobile phase: supercritical CO₂/EtOH (0.1% NH₃·H₂O, v %)=40/40; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm) to afford Compound ent-12 and Compound 12.

### Compound Ent-12

2-methoxy-5-[[2-oxo-2-[(2R,5S)-5-methyl-2-[2-[rel-(3R,4S)-1,3-dimethyl-4-piperidyl]-1,3-benzothiazol-5-yl]-1-piperidyl]acetyl]amino]pyridine-3-carboxamide (40 mg, peak1, Rentention time=3.651 min, single unknown enantiomer with trans relative chemistry, white solid).

¹H NMR (400 MHz, methanol-d₄) δ ppm 8.38-8.90 (m, 2H), 7.84-8.11 (m, 2H), 7.46 (br d, J 8.5 Hz, 1H), 5.43-5.96 (m, 1H), 4.02-4.22 (m, 3H), 3.40-3.86 (m, 1H), 3.13 (br t, J=13.3 Hz, 2H), 2.85 (br d, J 9.3 Hz, 1H), 2.47 (s, 3H), 1.78-2.42 (m, 9H), 1.48 (br d, J=13.1 Hz, 1H), 1.15 (d, J 6.8 Hz, 3H), 0.88 (br d, J 5.5 Hz, 3H); LCMS (ESI) [M+H]⁺ m/z: calcd 565.3, found 565.3; HPLC: 99.9%@254 nm; 98.86% ee.

### Compound 12

2-methoxy-5-[[2-oxo-2-[(2R,5S)-5-methyl-2-[2-[rel-3R,4R)-1,3-dimethyl-4-piperidyl]-1,3-benzothiazol-5-yl]-1-piperidyl]acetyl]amino]pyridine-3-carboxamide (11 mg, peak2, Rentention time=4.177 min, single unknown enantiomer with trans relative chemistry, white solid).

¹H NMR (400 MHz, methanol-d₄) δ ppm 8.38-8.72 (m, 1H), 7.84-8.06 (m, 2H), 7.26-7.71 (m, 2H), 5.36-6.04 (m, 1H), 3.98-4.27 (m, 3H), 3.35-3.88 (m, 2H), 2.77-3.27 (m, 2H), 1.81-2.58 (m, 9H), 1.48 (br d, J 12.8 Hz, 1H), 1.28 (s, 1H), 1.15 (d, J 7.0 Hz, 3H), 0.93 (br d, J 7.0 Hz, 3H); LCMS (ESI) [M+H]⁺ m/z: calcd 565.3, found 565.4; HPLC: 100.0%@254 nm; 99.4% ee.

### Example 14. Compound 19 N-(6-amino-5-ethyl-3-pyridyl)-2-oxo-2-[(2R,5S)-5-methyl-2-[3-methyl-2-(1-methyl-4-piperidyl)indazol-6-yl]-1-piperidyl]acetamide

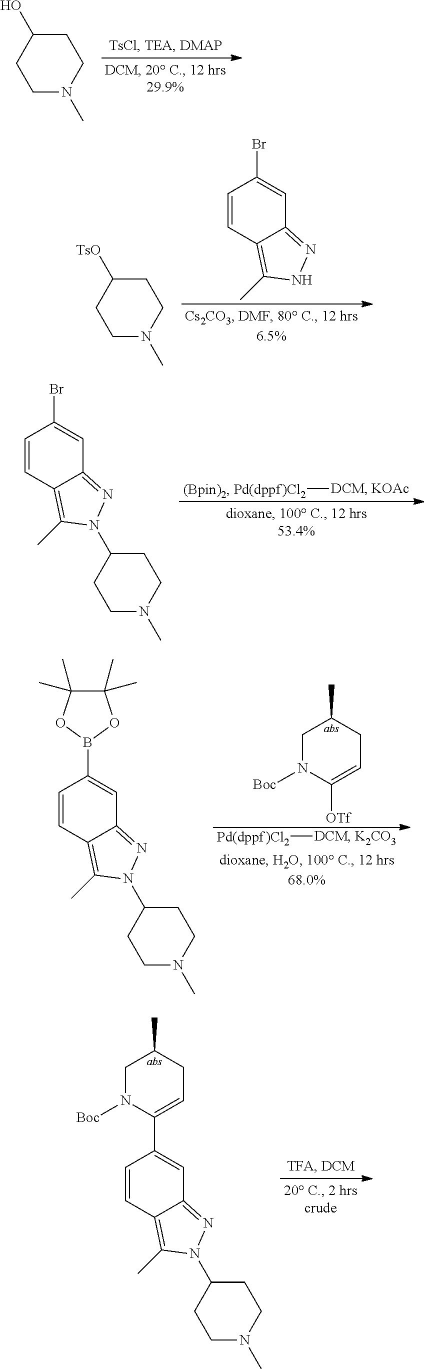

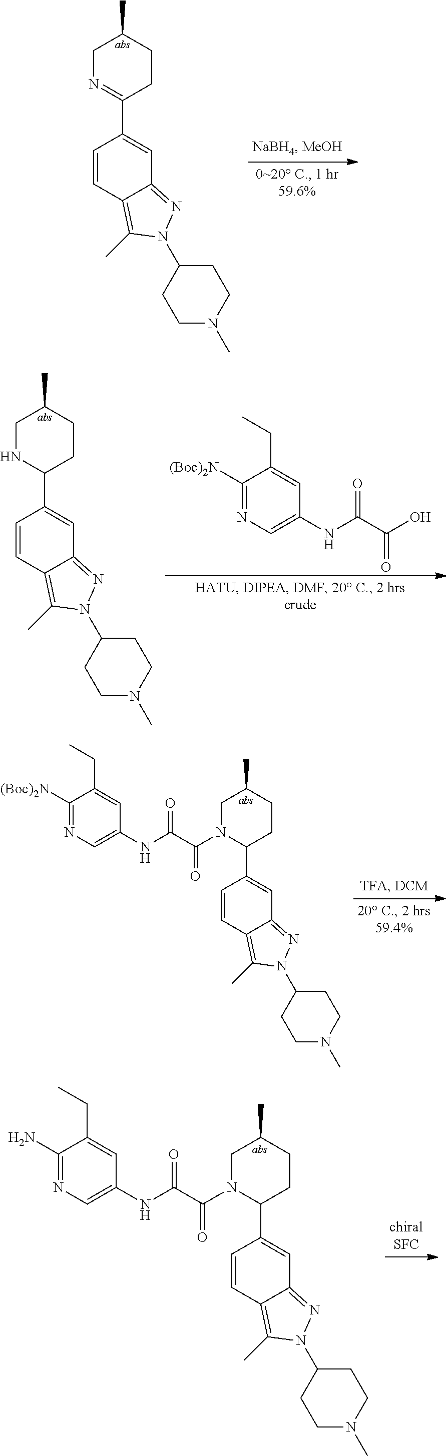

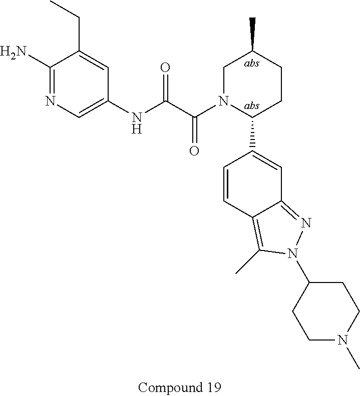 [SMILES: CCc1cc(NC(=O)C(=O)N2C[C@@H](C)CC[C@@H]2c2ccc3c(C)n(C4CCN(C)CC4)nc3c2)cnc1N]

### Step 1: Synthesis of (1-methyl-4-piperidyl) 4-methylbenzenesulfonate

To a solution of 1-methylpiperidin-4-ol (5 g, 43.41 mmol) in DCM (100 mL) was added TEA (0.130 mol, 18.2 mL), DMAP (1.06 g, 8.68 mmol) and 4-methylbenzenesulfonyl chloride (9.10 g, 47.8 mmol). The mixture was stirred at 20° C. for 12 hours. The resulting mixture was quenched by addition of water (50 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure. The residue was purified by flash chromatography (ISCO®; 40 g AgelaFlash® Silica Flash Column, petroleummether/EtOAc with EtOAc from 0˜30%, flow rate=40 mL/min, I₂) to afford (1-methyl-4-piperidyl) 4-methylbenzenesulfonate (3.5 g, 29.9% yield) as colorless oil.

### Step 2: Synthesis of 6-bromo-3-methyl-1-(1-methyl-4-piperidyl)indazole

To a solution of 6-bromo-3-methyl-2H-indazole (3 g, 14.2 mmol) in DMF (30 mL) was added (1-methyl-4-piperidyl) 4-methylbenzenesulfonate (4.21 g, 15.6 mmol) and Cs₂CO₃ (13.9 g, 42.6 mmol). The mixture was stirred at 90° C. for 12 hours under N₂ atmosphere. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure. The residue was purified by flash chromatography (ISCO®; 40 g AgelaFlash® Silica Flash Column, petroleum ether/EtOAc with EtOAc from 0˜30%, flow rate=30 mL/min, 254 nm) to afford a mixture of 6-bromo-3-methyl-2-(1-methyl-4-piperidyl)indazole (2.1 g, 47.9% yield) and 6-bromo-3-methyl-1-(1-methyl-4-piperidyl)indazole (2 g, crude) as brown oil, which was purified by preparative HPLC (Instrument: Gilson GX-281 Liquid Handler, Gilson 322 Pump, Gilson 156 UV Detector; Column:2_Phenomenex Gemini Cis 75*40 mm*3 m; Mobile phase A: H₂O with 0.05% NH₃—H₂O (v %); Mobile phase B: MeCN; Gradient: B from 37% to 67% in 9.5 min, hold 100% B for 2 min; Flow Rate: 25 mL/min; Column Temperature: 30° C.; Wavelength: 220 nm, 254 nm) to afford 6-bromo-3-methyl-2-(1-methyl-4-piperidyl)indazole (130 mg, 6.50% yield) as a white solid. ¹H NMR (400 MHz, methanol-d₄) δ ppm 7.70 (d, J=1.0 Hz, 1H), 7.56 (d, J=8.9 Hz, 1H), 7.08 (dd, J=8.9, 1.6 Hz, 1H), 4.46-4.57 (m, 1H), 3.05-3.13 (m, 2H), 2.65 (s, 3H), 2.30-2.43 (m, 7H), 1.91-2.14 (m, 2H).

### Step 3: Synthesis of 3-methyl-2-(1-methyl-4-piperidyl)-6-(4,4,5,5-tetramethyl-1,3,2-dioxaborolan-2-yl)indazole

To a solution of 6-bromo-3-methyl-2-(1-methyl-4-piperidyl)indazole (130 mg, 0.421 mmol) in dioxane (5 mL) was added (Bpin)₂ (156 mg, 0.614 mmol), KOAc (125 mg, 1.27 mmol) and Pd(dppf)Cl₂-DCM (36 mg, 0.0441 mmol). The mixture was stirred at 100° C. for 12 hours under nitrogen atmosphere. The resulting mixture was quenched by addition of water (30 mL) and extracted with EtOAc (100 mL*3). The combined organic layer was washed with brine (50 mL), dried over anhydrous Na₂SO₄, filtered and concentrated under reduced pressure. The residue was purified by flash chromatography (ISCO®; 4 g AgelaFlash® Silica Flash Column, DCM/MeOH with MeOH from 0˜10%, flow rate=30 mL/min, 254 nm) to afford 3-methyl-2-(1-methyl-4-piperidyl)-6-(4,4,5,5-tetramethyl-1,3,2-dioxaborolan-2-yl)indazole (80 mg, 53.4% yield) as yellow solid. LCMS (ESI) [M+H]⁺ m/z: calcd 356.2, found 356.2.

### Step 4: Synthesis of tert-butyl (3S)-3-methyl-6-[3-methyl-2-(1-methyl-4-piperidyl)indazol-6-yl]-3,4-dihydro-2H-pyridine-1-carboxylate

A mixture of 3-methyl-2-(1-methyl-4-piperidyl)-6-(4,4,5,5-tetramethyl-1,3,2-dioxaborolan-2-yl)indazole (80 mg, 0.225 mmol), tert-butyl (3S)-3-methyl-6-(trifluoromethylsulfonyloxy)-3,4-dihydro-2H-pyridine-1-carboxylate (105 mg, 0.304 mmol), K₂CO₃ (90 mg, 0.651 mmol), Pd(dppf)Cl₂-DCM (26 mg, 0.0318 mmol) in dioxane (3 mL) and H₂O (1 mL) was stirred at 100° C. for 12 hours under nitrogen. The resulting mixture was concentrated under reduced pressure to give residue which was purified by flash chromatography (ISCO®; 4 g AgelaFlash® Silica Flash Column, DCM/MeOH with MeOH from 0˜10%, flow rate=30 mL/min, 254 nm) to afford tert-butyl (3S)-3-methyl-6-[3-methyl-2-(1-methyl-4-piperidyl)indazol-6-yl]-3,4-dihydro-2H-pyridine-1-carboxylate (65 mg, 68.0% yield) as yellow oil. LCMS (ESI) [M+H]⁺ m/z: calcd 425.3, found 425.3.

### Step 5: Synthesis of 3-methyl-2-(1-methyl-4-piperidyl)-6-[(3S)-3-methyl-2,3,4,5-tetrahydropyridin-6-yl]indazole

A mixture of tert-butyl (3S)-3-methyl-6-[3-methyl-2-(1-methyl-4-piperidyl)indazol-6-yl]-3,4-dihydro-2H-pyridine-1-carboxylate (65 mg, 0.153 mmol), DCM (1 mL) and TFA (1 mL, 13.0 mmol) was stirred at 20° C. for 2 hours. The resulting mixture was concentrated under reduced pressure to give 3-methyl-2-(1-methyl-4-piperidyl)-6-[(3S)-3-methyl-2,3,4,5-tetrahydropyridin-6-yl]indazole (50 mg, crude) as brown oil.

### Step 6: Synthesis of 3-methyl-2-(1-methyl-4-piperidyl)-6-[(5S)-5-methyl-2-piperidyl]indazole

To a mixture of 3-methyl-2-(1-methyl-4-piperidyl)-6-[(3S)-3-methyl-2,3,4,5-tetrahydropyridin-6-yl]indazole (50 mg, 0.154 mmol) in MeOH (5 mL) was adjusted to pH=7 with Na₂CO₃, then NaBH₄ (10 mg, 0.264 mmol) was added at 0° C. slowly. The resulting mixture was stirred at 20° C. for 1 hour. The resulting mixture was quenched by addition of water (10 mL) and concentrated under reduced pressure to give a crude product which was purified by flash chromatography (Column: SepaFlash® Sphercial C18, 60 g, 40-60 m, 120A; MeCN/water (0.05 v % NH₃—H₂O) with MeCN from 0-50%, 25 mL/min, 254 nm) to afford 3-methyl-2-(1-methyl-4-piperidyl)-6-[(5S)-5-methyl-2-piperidyl]indazole (30 mg, 59.6% yield) as a yellow oil. LCMS (ESI) [M+H]⁺ m/z: calcd 327.3, found 327.2.

### Step 7: Synthesis of tert-butyl N-tert-butoxycarbonyl-N-[3-ethyl-5-[[2-oxo-2-[(5S)-5-methyl-2-[3-methyl-2-(1-methyl-4-piperidyl)indazol-6-yl]-1-piperidyl]acetyl]amino]-2-pyridyl]carbamate

A mixture of 3-methyl-2-(1-methyl-4-piperidyl)-6-[(5S)-5-methyl-2-piperidyl]indazole (30 mg, 91.9 μmol) and 2-[[6-[bis(tert-butoxycarbonyl)amino]-5-ethyl-3-pyridyl]amino]-2-oxo-acetic acid (45 mg, 0.110 mmol) in DMF (2 mL) were added HATU (42 mg, 0.110 mmol) and DIPEA (0.050 mL, 0.287 mmol,). The resulting mixture was stirred at 20° C. for 2 hours. The resulting mixture was concentrated under reduced pressure to give tert-butyl N-tert-butoxycarbonyl-N-[3-ethyl-5-[[2-oxo-2-[(5S)-5-methyl-2-[3-methyl-2-(1-methyl-4-piperidyl)indazol-6-yl]-1-piperidyl]acetyl]amino]-2-pyridyl]carbamate (70 mg, crude) as yellow oil, which was used into the next step without further purification.

### Step 8: Synthesis of N-(6-amino-5-ethyl-3-pyridyl)-2-oxo-2-[(5S)-5-methyl-2-[3-methyl-2-(1-methyl-4-piperidyl)indazol-6-yl]-1-piperidyl]acetamide

A mixture of tert-butyl N-tert-butoxycarbonyl-N-[3-ethyl-5-[[2-oxo-2-[(5S)-5-methyl-2-[3-methyl-2-(1-methyl-4-piperidyl)indazol-6-yl]-1-piperidyl]acetyl]amino]-2-pyridyl]carbamate (70 mg, 97.5 μmol), DCM (1 mL) and TFA (1 mL, 13.0 mmol) was stirred at 20° C. for 2 hours. The resulting mixture was adjusted to pH=9 with NH₃—H₂O (12 N), and then the mixture was concentrated in vacuo to give crude product, which was purified by preparative HPLC (Instrument: Gilson GX-281 Liquid Handler, Gilson 322 Pump, Gilson 156 UV Detector; Column: 2_Phenomenex Gemini Cis 75*40 mm*3 um; Mobile phase A: H₂O with 0.05% NH₃—H₂O (v %); Mobile phase B: MeCN; Gradient: B from 32% to 62% in 9.5 min, hold 100% B for 2 min; Flow Rate: 25 mL/min; Column Temperature: 30° C.; Wavelength: 220 nm, 254 nm) to afford N-(6-amino-5-ethyl-3-pyridyl)-2-oxo-2-[(5S)-5-methyl-2-[3-methyl-2-(1-methyl-4-piperidyl)indazol-6-yl]-1-piperidyl]acetamide (30 mg, 59.4% yield) as a white solid. LCMS (ESI) [M+H]⁺ m/z: calcd 518.3, found 518.3.

### Step 9: Synthesis of N-(6-amino-5-ethyl-3-pyridyl)-2-oxo-2-[(2R,5S)-5-methyl-2-[3-methyl-2-(1-methyl-4-piperidyl)indazol-6-yl]-1-piperidyl]acetamide

N-(6-amino-5-ethyl-3-pyridyl)-2-oxo-2-[(5S)-5-methyl-2-[3-methyl-2-(1-methyl-4-piperidyl)indazol-6-yl]-1-piperidyl]acetamide (30 mg, 57.9 mol) was purified by SFC (Instrument: Berger, MULTIGR AM-II; Column: DAICEL CHIRALPAK AS (250 mm*30 mm*5 um); Mobile phase: supercritical Hexane-IPA (0.1% NH₃, MeOH v %)=60/40; Flow Rate:80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm). The fraction was concentrated under reduced pressure and then lyophilized for overnight.

Compound 19: N-(6-amino-5-ethyl-3-pyridyl)-2-oxo-2-[(2R,5S)-5-methyl-2-[3-methyl-2-(1-methyl-4-piperidyl)indazol-6-yl]-1-piperidyl]acetamide (17.8 mg, single known enantiomer with trans relative chemistry, Peak 2, Retention time: 5.667 min, white solid). ¹H NMR (400 MHz, methanol-d₄) δ ppm 7.87-8.12 (m, 1H), 7.64 (br s, 2H), 7.52 (br s, 1H), 6.98-7.11 (m, 1H), 5.37-5.78 (m, 1H), 3.70-4.07 (m, 1H), 3.42 (br d, J=8.5 Hz, 1H), 3.08 (br d, J=8.9 Hz, 2H), 2.66 (br s, 3H), 2.52 (br d, J=7.4 Hz, 2H), 2.32-2.45 (m, 10H), 1.99 (br s, 4H), 1.46 (br d, J=10.5 Hz, 1H), 1.25 (br d, J=7.1 Hz, 3H), 1.13 (br d, J=6.8 Hz, 3H); LCMS (ESI) [M+H]⁺ m/z: calcd 518.3, found 518.3; HPLC: 100%@254 nm; SFC: 99.7% ee.

### Example 15. Compound 110 (5-amino-6-methyl-1H-pyrrolo[3,2-b]pyridin-2-yl)((2R,5S)-5-methyl-2-(2-(1-methylpiperidin-4-yl)benzo[d]thiazol-5-yl)piperidin-1-yl)methanone

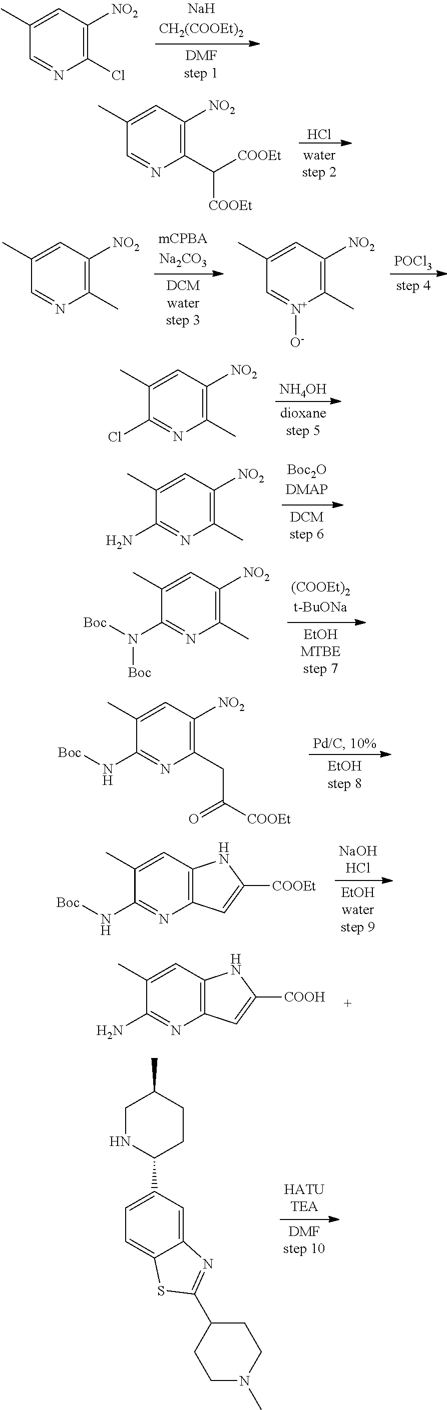

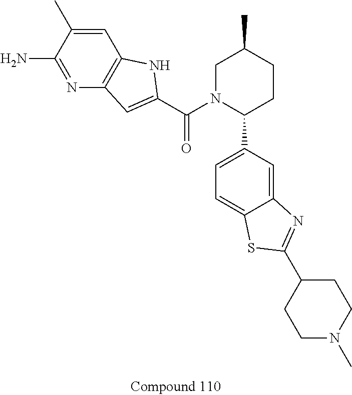 [SMILES: Cc1cc2[nH]c(C(=O)N3C[C@@H](C)CC[C@@H]3c3ccc4sc(C5CCN(C)CC5)nc4c3)cc2nc1N]

### Step 1: Synthesis of diethyl 2-(5-methyl-3-nitropyridin-2-yl)malonate

To a stirred slurry of sodium hydride (in oil dispersion) 60% dispersion in mineral oil (12.17 g, 304.23 mmol, 60% purity) in DMF (250 mL), diethyl malonate (46.41 g, 289.74 mmol, 43.99 mL) was added dropwise under argon at 5-10° C. Then a solution of 2-chloro-5-methyl-3-nitro-pyridine (25 g, 144.87 mmol) in DMF (100 mL) was added dropwise and the reaction mixture was slowly warmed to 80° C. and stirred at this temperature for 5 hr. The reaction mixture was cooled down, poured in water (1200 ml) and acidified to pH5-6 with 2N aqueous hydrochloric acid. The resulting mixture was extracted with MTBE (2*400 ml). The combined MTBE extracts were washed with water (3*100 ml), dried over sodium sulfate and concentrated in vacuum to afford crude diethyl 2-(5-methyl-3-nitro-2-pyridyl)propanedioate (64 g, crude) as yellow oil (almost pure product in mixture with diethyl malonate), which was used directly in the next step.

LCMS(ESI): [M]⁺ m/z: calcd 296.2; found 297.2; Rt=1.081 min.

### Step 2: Synthesis of 2,5-dimethyl-3-nitropyridine

Diethyl 2-(5-methyl-3-nitro-2-pyridyl)propanedioate (64 g, 216.02 mmol) was added to a mixture of hydrochloric acid, 36% w/w aq. soln. (295.00 g, 2.91 mol, 250 mL, 36% purity) and water (250 mL). The resulting emulsion was stirred vigorously with a reflux condenser at 125° C. for 15 hr. Then the resulting mixture (yellow solution with oily grease from sodium hydride (from previous step)) was cooled down and basified to pH 8 with 20% aqueous sodium hydroxide solution (crushed ice was added to maintain temperature below 40° C.). The resulting oily product was extracted with dichloromethane (2*300 ml). The combined organic extracts were dried over sodium sulfate and concentrated in vacuum to afford crude 2,5-dimethyl-3-nitro-pyridine (22 g, 144.59 mmol, 66.94% yield) as yellow gum, which was used directly in the next step.

LCMS(ESI): [M]⁺ m/z: calcd 152.2; found 153.2; Rt=0.835 min.

### Step 3: Synthesis of 2,5-dimethyl-3-nitropyridine 1-oxide

3-Chloroperbenzoic acid (30 g, 139.08 mmol, 80% purity) was added portion wise to a cooled to 0° C. and stirred solution of 2,5-dimethyl-3-nitro-pyridine (22 g, 144.59 mmol) in DCM (500 mL). The resulting mixture was allowed to warm to 25° C. and stirred for 15 hr. Then a solution of sodium carbonate (15.33 g, 144.59 mmol, 6.05 mL) in water (150 mL) was slowly added to the reaction mixture (foaming!). The resulting mixture was additionally shaked in a separatory funnel (gently!), the organic layer was separated, dried over sodium sulfate and concentrated in vacuum to afford 2,5-dimethyl-3-nitro-1-oxido-pyridin-1-ium (21 g, 124.89 mmol, 86.37% yield) as yellow solid, which was used directly in the next step.

LCMS(ESI): [M]⁺ m/z: calcd 168.2; found 169.2; Rt=0.702 min.

### Step 4: Synthesis of 2-chloro-3,6-dimethyl-5-nitropyridine

2,5-Dimethyl-3-nitro-1-oxido-pyridin-1-ium (18 g, 107.05 mmol) was slowly added to a phosphorus(V) oxychloride (164.00 g, 1.07 mol, 100 mL) at 25° C. with stirring. The resulting mixture was stirred with a reflux condenser at 100° C. for 3 hr. The resulting solution was cooled down and concentrated in vacuum. Crushed ice was added to the residue, and the resulting mixture was extracted with DCM (2*100 ml). The combined organic extracts were washed with ice-cold water (2*50 ml), dried over sodium sulfate and concentrated in vacuum to afford crude product (17.5 g), which was purified by column chromatography on silica gel using hexane/MTBE gradient (0-100% MTBE) to afford 2-chloro-3,6-dimethyl-5-nitro-pyridine (7 g, 37.51 mmol, 35.04% yield) as yellow solid.

LCMS(ESI): [M]⁺ m/z: calcd 186.2; found 187.2; Rt=1.124 min.

### Step 5: Synthesis of 3,6-dimethyl-5-nitropyridin-2-amine

Ammonium hydroxide, 28% NH₃ (54.00 g, 1.54 mol, 60 mL) was added in one portion to a stirred solution of 2-chloro-3,6-dimethyl-5-nitro-pyridine (7 g, 37.51 mmol) in dioxane (40 mL). The resulting mixture was stirred in autoclave at 90° C. for 60 hr, then cooled down and concentrated to dryness in vacuum. The residue was diluted with water (50 ml), the precipitate was filtered, washed successively with water (2*10 ml) and MTBE (2*5 ml), and air-dried to afford 3,6-dimethyl-5-nitro-pyridin-2-amine (5.7 g, 34.10 mmol, 90.89% yield) as light-yellow solid.

LCMS(ESI): [M]⁺ m/z: calcd 167.2; found 168.2; Rt=0.641 min.

### Step 6: Synthesis of tert-butyl N-tert-butoxycarbonyl-N-(3,6-dimethyl-5-nitro-2-pyridyl)carbamate

Di-tert butyl dicarbonate (15.08 g, 69.09 mmol, 15.86 mL) was added in one portion at 25° C. to a stirred slurry of 3,6-dimethyl-5-nitro-pyridin-2-amine (5.5 g, 32.90 mmol) and DMAP (137.50 mg, 1.13 mmol) in DCM (100 mL). The resulting mixture was stirred at 25° C. for 16 hr to form clear solution. The reaction mixture was transferred to a separatory funnel, washed with water (2*50 ml), dried over sodium sulfate and concentrated in vacuum to afford tert-butyl N-tert-butoxycarbonyl-N-(3,6-dimethyl-5-nitro-2-pyridyl)carbamate (12 g, 32.66 mmol, 99.27% yield) as light-yellow solid.

LCMS(ESI): [M-t-Bu]⁺ m/z: calcd 311.2; found 312.2; Rt=1.451 min.

### Step 7: Synthesis of ethyl 3-(6-((tert-butoxycarbonyl)amino)-5-methyl-3-nitropyridin-2-yl)-2-oxopropanoate

tert-Butyl N-tert-butoxycarbonyl-N-(3,6-dimethyl-5-nitro-2-pyridyl)carbamate (2.75 g, 7.49 mmol) was added in one portion at 25° C. to a freshly prepared solution of sodium tert-butoxide (1.65 g, 17.17 mmol) in absolute EtOH (30 mL). The resulting mixture was stirred at 25° C. for 15 min (red solution formed), then diethyl oxalate (2.6 g, 17.79 mmol, 2.41 mL) was added in one portion and the reaction mixture was stirred at 25° C. for 120 hr, then diluted with MTBE (70 mL). The resulting precipitate was filtered, then suspended in water (20 ml) and carefully acidified to pH 3-4 with 1N aqueous hydrochloric acid. The resulting precipitate was filtered, washed with water (2*5 ml) and air dried to afford ethyl 3-[6-(tert-butoxycarbonylamino)-5-methyl-3-nitro-2-pyridyl]-2-oxo-propanoate (1 g, 2.72 mmol, 36.37% yield) as yellow solid.

LCMS(ESI): [M]⁺ m/z: calcd 367.2; found 368.2; Rt=1.404 min.

### Step 8: Synthesis of ethyl 5-((tert-butoxycarbonyl)amino)-6-methyl-1H-pyrrolo[3,2-b]pyridine-2-carboxylate

A mixture of ethyl 3-[6-(tert-butoxycarbonylamino)-5-methyl-3-nitro-2-pyridyl]-2-oxo-propanoate (900 mg, 2.45 mmol) and palladium, 10% on carbon (50 mg, 2.45 mmol) in EtOH (5 mL) was stirred under atmosphere of hydrogen (balloon pressure) at 50° C. for 24 hr. The reaction mixture was cooled, and isolated. The palladium catalyst was filtered, the filtrate was concentrated in vacuum to afford crude ethyl 5-(tert-butoxycarbonylamino)-6-methyl-1H-pyrrolo[3,2-b]pyridine-2-carboxylate (900 mg, crude) as light-yellow gum, which was used directly in the next step.

LCMS(ESI): [M-t-Bu]⁺ m/z: calcd 263.2; found 264.2; Rt=2.362 min.

### Step 9: Synthesis of 5-amino-6-methyl-1H-pyrrolo[3,2-b]pyridine-2-carboxylic acid

A solution of sodium hydroxide, pearl (450.88 mg, 11.27 mmol, 211.68 μL) in water (2.98 mL) was added in one portion to a stirred solution of ethyl 5-(tert-butoxycarbonylamino)-6-methyl-1H-pyrrolo[3,2-b]pyridine-2-carboxylate (900 mg, 2.82 mmol) in EtOH (9.93 mL). The resulting mixture was stirred at 25° C. for 12 hr and then concentrated in vacuum. The residue was diluted with water (10 ml) and acidified with 6N aqueous hydrochloric acid to pH 4-5. The resulting mixture was concentrated to dryness in vacuum. The H-NMR showed only partial boc-protection cleavage, ester remained unchanged. The residue was diluted with a solution of hydrochloric acid, 36% w/w aq. soln. (5.90 g, 161.82 mmol, 5 mL) in water (20 mL). The resulting mixture was stirred at 70° C. for 12 hr (after approximately reaction mixture became a solution). After 12 hr precipitate was formed. The reaction mixture was cooled to 25° C., the precipitate was filtered, washed with water (2*1 ml) and dried at 60° C. to afford 5-amino-6-methyl-1H-pyrrolo[3,2-b]pyridine-2-carboxylic acid (200 mg, 878.55 mol, 31.17% yield, HCl) as white solid.

LCMS(ESI): [M]⁺ m/z: calcd 191.2; found 192.2; Rt=0.556 min.

### Step 10: Synthesis of (5-amino-6-methyl-1H-pyrrolo[3,2-b]pyridin-2-yl)((2R,5S)-5-methyl-2-(2-(1-methylpiperidin-4-yl)benzo[d]thiazol-5-yl)piperidin-1-yl)methanone

Prepared by general procedure scheme 4.1 step 6A. Yield: 49.7 mg (22.51%).

HPLC conditions: Column: XBridge C18 100*19 mm, 5 microM; 0-5 min 40-90% water-MeOH+0.1% NH₄OH, flow: 30 ml/min; (loading pump 4 ml/min MeCN).

Compound 110: ¹H NMR (600 MHz, dmso) δ 1.01 (d, 3H), 1.27-1.34 (m, 1H), 1.64-1.72 (m, 1H), 1.76-1.83 (m, 2H), 1.88-2.08 (m, 6H), 2.12 (s, 3H), 2.17 (s, 3H), 2.19-2.29 (m, 2H), 2.80-2.86 (m, 2H), 3.00-3.08 (m, 1H), 4.17 (d, 1H), 5.26 (s, 2H), 5.82 (t, 1H), 6.31 (s, 1H), 7.31 (s, 1H), 7.38 (dd, 1H), 7.85 (s, 1H), 8.06 (d, 1H), 11.29 (d, 1H).

LCMS(ESI): [M]⁺ m/z: calcd 502.2; found 503.2; Rt=2.222 min.

### Example 16. Compound 110 (5-amino-6-methyl-1H-pyrrolo[3,2-b]pyridin-2-yl)-[(2R,5S)-5-methyl-2-[2-(1-methyl-4-piperidyl)-1,3-benzothiazol-5-yl]-1-piperidyl]methanone

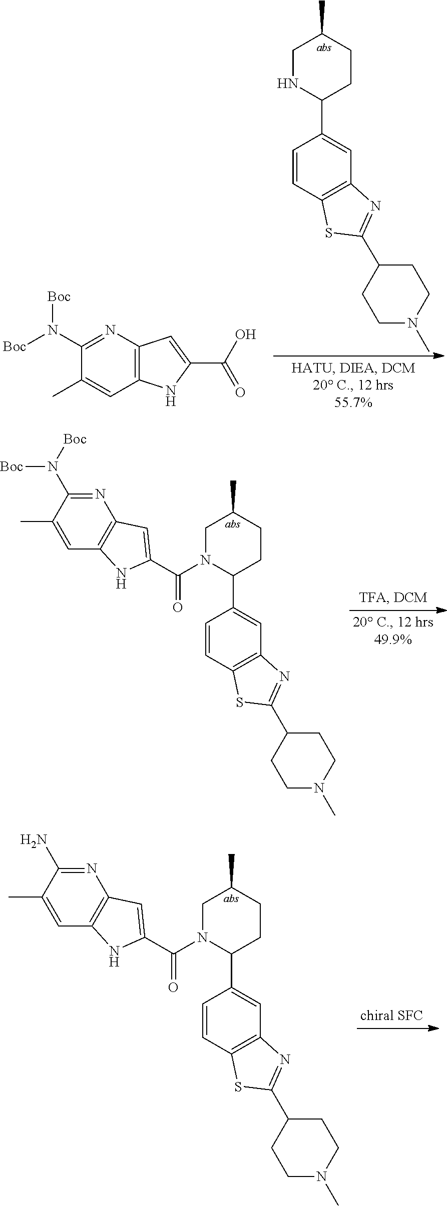

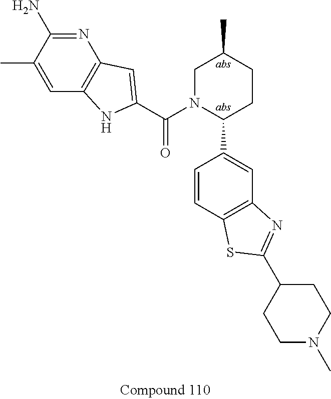

### Step 1: Synthesis of tert-butyl N-tert-butoxycarbonyl-N-[6-methyl-2-[(5S)-5-methyl-2-[2-(1-methyl-4-piperidyl)-1,3-benzothiazol-5-yl]piperidine-1-carbonyl]-1H-pyrrolo[3,2-b]pyridin-5-yl]carbamate

To a mixture of 5-[bis(tert-butoxycarbonyl)amino]-6-methyl-1H-pyrrolo[3,2-b]pyridine-2-carboxylic acid (200 mg, 0.511 mmol), 2-(1-methyl-4-piperidyl)-5-[(5S)-5-methyl-2-piperidyl]-1,3-benzothiazole (200 mg, 0.607 mmol), HATU (300 mg, 0.789 mmol), N-ethyl-N-isopropyl-propan-2-amine (0.5 mL, 2.87 mmol) and DCM (5 mL) was stirred at 20° C. for 12 hours. The resulting mixture was quenched by addition of water (50 mL) and extracted with DCM (50 mL*3). The combined organic layer was washed with saturated NH₄Cl aqueous solution (50 mL*2), brine (50 mL), dried over anhydrous Na₂SO₄, filtered and concentrated under reduced pressure. The residue was purified by flash chromatography (ISCO®; 12 g AgelaFlash® Silica Flash Column, EtOAc/MeOH with MeOH from 0˜30%, flow rate=30 mL/min, 254 nm) to afford tert-butyl N-tert-butoxycarbonyl-N-[6-methyl-2-[(5S)-5-methyl-2-[2-(1-methyl-4-piperidyl)-1,3-benzothiazol-5-yl]piperidine-1-carbonyl]-1H-pyrrolo[3,2-b]pyridin-5-yl]carbamate (200 mg, 55.7% yield) as yellow oil. LCMS (ESI) [M+H]⁺ m/z: calcd 703.4, found 703.4.

### Step 2: Synthesis of (5-amino-6-methyl-1H-pyrrolo[3,2-b]pyridin-2-yl)-[(5S)-5-methyl-2-[2-(1-methyl-4-piperidyl)-1,3-benzothiazol-5-yl]-1-piperidyl]methanone

To a solution of tert-butyl N-tert-butoxycarbonyl-N-[6-methyl-2-[(5S)-5-methyl-2-[2-(1-methyl-4-piperidyl)-1,3-benzothiazol-5-yl]piperidine-1-carbonyl]-1H-pyrrolo[3,2-b]pyridin-5-yl]carbamate (200 mg, 0.199 mmol) in DCM (5 mL) was added TFA (0.15 mL, 1.99 mmol). The mixture was stirred at 20° C. for 12 hours. The resulting mixture was filtered and concentrated under reduced pressure. The residue was purified by preparative HPLC (Instrument: Gilson GX-281 Liquid Handler, Gilson 322 Pump, Gilson 156 UV Detector; Column: Phenomenex Gemini-NX 80×40 mm×3 m; Mobile phase A: H₂O with 10 mm NH₄HCO₃ (v %); Mobile phase B: MeCN; Gradient: B from 22% to 52% in 7.8 min, hold 100% B for 1 min; Flow Rate: 25 mL/min; Column Temperature: 30° C.; Wavelength: 220 nm, 254 nm) to afford (5-amino-6-methyl-1H-pyrrolo[3,2-b]pyridin-2-yl)-[(5S)-5-methyl-2-[2-(1-methyl-4-piperidyl)-1,3-benzothiazol-5-yl]-1-piperidyl]methanone (50 mg, 49.9% yield) as white solid. LCMS (ESI) [M+H]⁺ m/z: calcd 503.3, found 503.3; HPLC: 98.80%@220 nm, 98.93%@254 nm.

### Step 3: Synthesis of (5-amino-6-methyl-1H-pyrrolo[3,2-b]pyridin-2-yl)-[(2R,5S)-5-methyl-2-[2-(1-methyl-4-piperidyl)-1,3-benzothiazol-5-yl]-1-piperidyl]methanone (Compound 110)

(5-amino-6-methyl-1H-pyrrolo[3,2-b]pyridin-2-yl)-[(5S)-5-methyl-2-[2-(1-methyl-4-piperidyl)-1,3-benzothiazol-5-yl]-1-piperidyl]methanone (50 mg, 0.0995 mmol) was purified by chiral SFC (Instrument: Berger, Multigr AM-II; Column: Chiralpak OD-3 (50 mm×4.6 mm×3 m; Mobile phase: supercritical CO₂/EtOH (0.05% DEA, v %)=60/40; Flow Rate: 4 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm to give Compound 110.

Compound 110: 5-amino-6-methyl-1H-pyrrolo[3,2-b]pyridin-2-yl)-[(2R,5S)-5-methyl-2-[2-(1-methyl-4-piperidyl)-1,3-benzothiazol-5-yl]-1-piperidyl]methanone (38 mg, peak 2, single known enantiomer with trans relative chemistry, Retention time: 1.486 min, white solid). ¹H NMR (400 MHz, methanol-d₄) δ ppm 8.01 (d, J=8.5 Hz, 1H), 7.94 (s, 1H), 7.53 (s, 1H), 7.46 (d, J=8.3 Hz, 1H), 6.48 (s, 1), 5.95 (s, 1H), 4.30 (d, J=13.8 Hz, 1H), 3.40 (dd, J=13.7, 3.1 Hz, 1H), 3.20-3.28 (m, 1H), 3.14 (d, J=12.3 Hz, 2H), 2.44-2.52 (m, 5H), 2.32-2.39 (m, 2H), 2.25 (s, 5H), 1.97-2.10 (m, 3H), 1.82-1.93 (m, 1H), 1.41-1.50 (m, 1H), 1.11 (d, J=7.0 Hz, 3H); LCMS (ESI) [M+H]⁺ m/z: calcd 503.3, found 503.2; HPLC: 100%@220 nm, 100%@254 nm; 99.8% ee.

### Example 17. Compound 51 N-(6-amino-5-iodopyridin-3-yl)-2-((2R,5S)-2-(benzo[d]thiazol-5-yl)-5-methylpiperidin-1-yl)-2-oxoacetamide

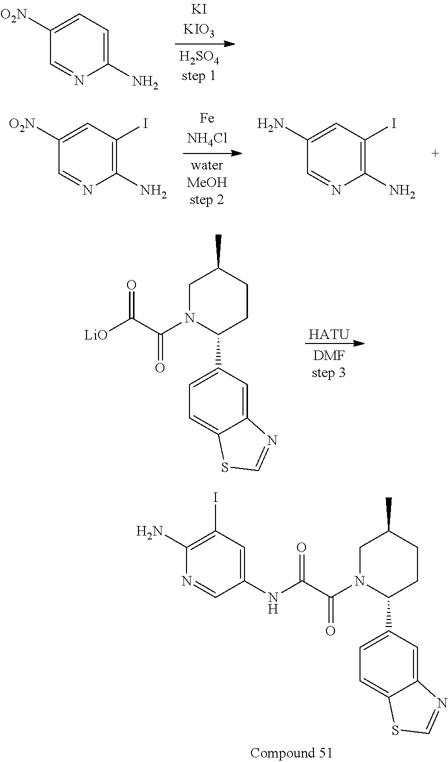

### Step 1: Synthesis of 3-10 do-5-nitropyridin-2-amine

5-Nitropyridin-2-amine (0.7 g, 5.03 mmol), potassium iodate (430.73 mg, 2.01 mmol) and potassium iodide, 99% (835.31 mg, 5.03 mmol, 267.73 μL) were mixed in sulfuric acid 2N (10 mL). The mixture was stirred at 80° C. overnight, after which Na₂CO₃ solution was added to adjust the pH to about 10. The resulting solids were collected by filtration, washed with water and ethanol successively and then dried in vacuum to give 3-iodo-5-nitro-pyridin-2-amine (1.2 g, 4.53 mmol, 89.99% yield).

LCMS(ESI): [M]⁺ m/z: calcd 265.2; found 266.2; Rt=1.079 min.

### Step 2: Synthesis of 3-iodopyridine-2,5-diamine

3-Iodo-5-nitro-pyridin-2-amine (0.5 g, 1.89 mmol), iron powder (1.05 g, 18.87 mmol, 134.06 μL) and ammonium chloride (1.01 g, 18.87 mmol, 659.62 μL) were added to the mixture of water (7 mL) and MeOH (7 mL). The resulting reaction mixture was stirred at 90° C. for 40 hr. After completion the precipitate was filtered of and the filtrate was evaporated under reduced pressure, the residue was diluted with EtOAc (50 mL) filtered through the thin pad of silica, the filtrate was evaporated dryness under reduced pressure to afford pure product 3-iodopyridine-2,5-diamine (50 mg, 212.74 mol, 11.28% yield).

LCMS(ESI): [M]⁺ m/z: calcd 235.2; found 236.2; Rt=0.289 min.

### Step 3: Synthesis of N-(6-amino-5-iodopyridin-3-yl)-2-((2R,5S)-2-(benzo[d]thiazol-5-yl)-5-methylpiperidin-1-yl)-2-oxoacetamide (Compound 51)

To a mixture of 3-iodopyridine-2,5-diamine (50 mg, 212.74 mol), [2-oxo-2-[(2R,5S)-2-(1,3-benzothiazol-5-yl)-5-methyl-1-piperidyl]acetyl]oxylithium (66.01 mg, 212.74 μmol) in DMF (4 mL), HATU (97.07 mg, 255.29 mol) was added in one portion. The resulting mixture was allowed to stir at 22° C. for 18 hr and then submitted to reverse phase HPLC (2-10 min 50-65% MeOH+NH₃ flow 30 ml/min (loading pump 4 ml MeOH), column: SunFire C18) to afford N-(6-amino-5-10 do-3-pyridyl)-2-oxo-2-[(2R,5S)-2-(1,3-benzothiazol-5-yl)-5-methyl-1-piperidyl]acetamide (58.8 mg, 112.78 μmol, 53.01% yield).

Compound 51: ¹H NMR (600 MHz, DMSO-d₆) δ (ppm) 1.02 (m, 3H), 1.36 (m, 1H), 1.71 (m, 1H), 1.88 (m, 1H), 2.14 (m, 1H), 2.31 (m, 1H), 2.81 (m, 1H), 3.78 (m, 1H), 5.52 (m, 1H), 5.96 (m, 2H), 7.46 (m, 1H), 8.02 (m, 1H), 8.17 (m, 3H), 9.40 (s, 1H), 10.75 (m, 1H).

LCMS(ESI): [M]⁺ m/z: calcd 521.2; found 522.2; Rt=3.208 min.

### Example 18. Compound 32 N-(6-amino-5-ethylpyridin-3-yl)-2-((2R,5S)-2-(2-cyanoquinolin-7-yl)-5-methylpiperidin-1-yl)-2-oxoacetamide

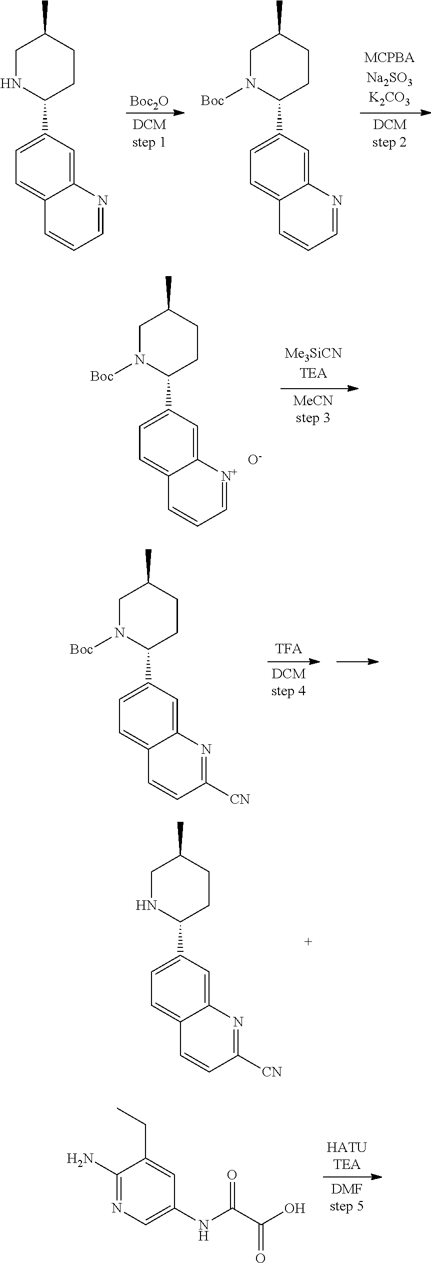

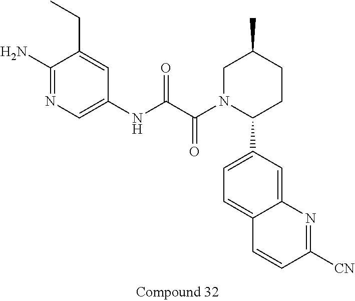 [SMILES: CCc1cc(NC(=O)C(=O)N2C[C@@H](C)CC[C@@H]2c2ccc3ccc(C#N)nc3c2)cnc1N]

### Step 1: Synthesis of (2R,5S)-tert-butyl 5-methyl-2-(quinolin-7-yl)piperidine-1-carboxylate

Di-tert-butyl dicarbonate (607.54 mg, 2.78 mmol, 638.84 μL) was added to the solution of 7-((2R,5S)-5-methylpiperidin-2-yl)quinolone (600 mg, 2.65 mmol) in DCM (15 mL). Resulting mixture was stirred at 25° C. for 14 hr. Then, volatiles were removed under reduced pressure and residue was crystallized from hexane (20 ml) to afford (2R,5S)-tert-butyl 5-methyl-2-(quinolin-7-yl)piperidine-1-carboxylate (475 mg, 1.46 mmol, 54.89% yield).

LCMS(ESI): [M]⁺ m/z: calcd 326.2; found 327.2; Rt=1.055 min.

### Step 2: Synthesis of 7-((2R,5S)-1-(tert-butoxycarbonyl)-5-methylpiperidin-2-yl)quinoline 1-oxide

MCPBA (408.05 mg, 1.89 mmol, 80% purity) was added portion wise to the solution of (2R,5S)-tert-butyl 5-methyl-2-(quinolin-7-yl)piperidine-1-carboxylate (475 mg, 1.46 mmol) in DCM (18.75 mL). Resulting mixture was stirred at 25° C. for 18 hr. Then, sodium sulfite (1 g, 7.93 mmol, 379.79 μL) and potassium carbonate (2 g, 14.47 mmol, 873.36 μL) dissolved in water (15 ml) were added to reaction mixture. Stirring was continued for 10 minutes and then, organic layer was separated, dried over Na₂SO₄ and concentrated under reduced pressure, affording 7-((2R,5S)-1-(tert-butoxycarbonyl)-5-methylpiperidin-2-yl)quinoline 1-oxide (530 mg, crude).

LCMS(ESI): [M]⁺ m/z: calcd 342.2; found 343.2; Rt=1.260 min.

### Step 3: Synthesis of (2R,5S)-tert-butyl 2-(2-cyanoquinolin-7-yl)-5-methylpiperidine-1-carboxylate

Trimethylsilyl cyanide (383.87 mg, 3.87 mmol) was added dropwise to the solution of 7-((2R,5S)-1-(tert-butoxycarbonyl)-5-methylpiperidin-2-yl)quinoline 1-oxide (530 mg, 1.55 mmol) and TEA (469.85 mg, 4.64 mmol, 647.18 μL) in MeCN (10 mL). Resulting mixture was stirred at 80° C. for 20 hr. Then, it was evaporated under reduced pressure, leaving (2R,5S)-tert-butyl 2-(2-cyanoquinolin-7-yl)-5-methylpiperidine-1-carboxylate (550 mg, crude).

LCMS(ESI): [M]⁺ m/z: calcd 351.2; found 352.2; Rt=1.504 min.

### Step 4: Synthesis of 7-((2R,5S)-5-methylpiperidin-2-yl)quinoline-2-carbonitrile

TFA (1.78 g, 15.65 mmol, 1.21 mL) was added to the solution of tert-butyl (2R,5S)-2-(2-cyano-7-quinolyl)-5-methyl-piperidine-1-carboxylate (550 mg, 1.56 mmol) in DCM (10 mL). Resulting mixture was stirred at 25° C. for 14 hr. Then, volatiles were removed under reduced pressure and residue was partitioned between 15% aq. K₂CO₃ (10 ml) solution and DCM (20 ml). Organic layer was separated, dried over K₂CO₃ and concentrated under reduced pressure, affording 7-[(2R,5S)-5-methyl-2-piperidyl]quinoline-2-carbonitrile (360 mg, 1.43 mmol, 91.53% yield).

LCMS(ESI): [M]⁺ m/z: calcd 251.2; found 252.2; Rt=0.867 min.

### Step 5: Synthesis of N-(6-amino-5-ethylpyridin-3-yl)-2-((2R,5S)-2-(2-cyanoquinolin-7-yl)-5-methylpiperidin-1-yl)-2-oxoacetamide (Compound 32)

Prepared by general procedure scheme 7.1 step 5A. Yield: 110 mg (34.71%).

HPLC conditions: Column: XBridge BEH C18 100*19 mm, 5 microM; 0-1-6 min 35-80% water-MeCN+0.1% NH₄OH 30 ml/min; (loading pump 4 ml/min MeCN).

Compound 32: ¹H NMR (600 MHz, DMSO-d₆) δ (ppm) 1.02-1.14 (m, 6H), 1.33-1.44 (m, 1H), 1.65-1.75 (m, 1H), 1.84-1.96 (m, 1H), 2.10-2.27 (m, 1H), 2.31-2.35 (m, 1H), 2.37-2.45 (m, 2H), 2.79-3.29 (m, 1H), 3.49-4.18 (m, 1H), 5.40-5.63 (m, 1H), 5.63-5.86 (m, 2H), 7.38-7.55 (m, 1H), 7.71-7.87 (m, 1H), 7.95-8.03 (m, 1H), 8.03-8.10 (m, 2H), 8.12-8.20 (m, 1H), 8.61-8.70 (m, 1H), 10.43-10.73 (m, 1H).

LCMS(ESI): [M]⁺ m/z: calcd 442.2; found 443.2; Rt=2.385 min.

### Scheme 4.1

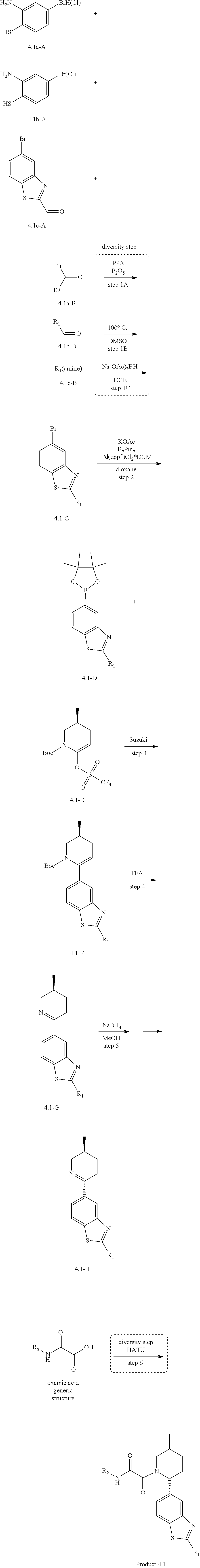

### Step 1A: Synthesis of 4.1-C

Phosphoric acid (4 eq) and phosphorus pentoxide (4 eq) were mixed together. The reaction suspension was stirred at rt for 10 min, then 4.1a-A (1 eq) followed by 4.1a-B (1.2 eq) were added under Ar. The solution was stirred at 110° C. for 18 hr then it was triturated with water, basidified (NaOH, 10% aq.) to pH=10, extracted with DCM twice, dried and evaporated in vacuum to give 4.1-C.

### Step 1B: Synthesis of 4.1-C

To the stirred solution of 4.1b-A (1 eq) in DMSO 4.1b-B (1 eq) was added. The resulting mixture was stirred at 100° C. for 14 hr. The reaction mixture was poured into cold water and extracted with MTBE twice. Combined organic layers were washed with water and brine, dried over Na₂SO₄. MTBE was evaporated in vacuum to give 4.1-C.

### Step 1C: Synthesis of 4.1-C

To the stirred solution of 4.1c-A (1 eq) in the 1,2-dichloroethane 4.1c-B (2 eq) was added and allowed to stir at 25° C. for 2 hr, sodium (trisacetoxy) borohydride (2 eq) was added. The reaction mixture was stirred at 25° C. for 16 hr. After completion, the reaction mixture was evaporated, quenched with water and neutralized by K₂CO₃ to pH=10. The aqueous phase was extracted with CHCl₃ twice. The combined organic phase was dried over Na₂SO₄ and evaporated under reduced pressure to afford 4.1-C. (TEA 1.5 eq per each acid eq, if amine salt used, was added to the solution of respective amine)

### Step 2: Synthesis of 4.1-D

4.1-C (1 eq), B₂Pin₂ (1.1 eq) and KOAc (2 eq) were mixed in dioxane. The resulting mixture was evacuated and then backfilled with argon, this operation was repeated three times, then Pd(dppf)Cl₂*DCM (0.05 eq) was added under argon. The reaction mixture was stirred under argon at 90° C. for 14 hr, then cooled and filtered. The filter cake was washed with dioxane twice. The solvent was evaporated to afford 4.1-D.

### Step 3: Synthesis of 4.1-F

4.1-D (1 eq), tert-butyl (3S)-3-methyl-6-(trifluoromethylsulfonyloxy)-3,4-dihydro-2H-pyridine-1-carboxylate (1.2 eq), sodium carbonate (3 eq) were mixed together in dioxane-water mixture (3:1). The resulting mixture was evacuated and then backfilled with argon. This operation was repeated two times, then Pd(dppf)Cl₂*DCM (819.86 mg, 1.00 mmol) was added and the reaction mixture was stirred under argon at 90° C. overnight, then cooled down and concentrated in vacuum. The residue was diluted with MTBE and stirred for 0.5 hr. After the most of the residue had dissolved, anhydrous sodium sulphate was added, and the resulting mixture was filtered. The filter cake was additionally washed with MTBE (5*50 ml) and discarded. The filtrate was concentrated in vacuum to afford 4.1-F.

### Step 4: Synthesis of 4.1-G

A solution of 4.1-F (1 eq) in TFA (15 eq) was stirred at rt for 1 hr, and then concentrated in vacuum. Cold water was added to the residue, and the resulting mixture was extracted with DCM twice. The DCM layer was discarded, and the aqueous layer was basified to pH 11. The resulting mixture was extracted with DCM twice. The combined organic extracts were dried over sodium sulphate and concentrated in vacuum to afford 4.1-G.

### Step 5: Synthesis of 4.1-H

4.1-G (1 eq) was dissolved in MeOH and the resulting solution was cooled to 0° C. in an ice bath. Sodium borohydride (2 eq) was added portion wise to the previous solution. After addition completed, the reaction mixture was allowed to warm to rt and stirred overnight. Water was added to the reaction mixture and the resulting mixture was concentrated in vacuum. The residue was diluted with water and the resulting mixture was extracted with DCM twice, dried over Na₂SO₄, filtered and evaporated to obtain 4.1-H.

### Step 6A: Synthesis of Product 4.1

4.1-H (1 eq), oxamic acid (1 eq) and TEA (2.5 eq+1.0 eq per each acid eq, if amine salt used) were mixed together in DMF. HATU (1.5 eq) was added thereto and the resulting mixture was stirred overnight. The reaction mixture was concentrated in vacuum and the residue was purified by HPLC to obtain Product 4.1.

### Step 6B: Synthesis of Product 4.1

DIPEA (2.5 eq+1.0 eq per each acid eq, if amine salt used) was added to the solution of respective amine or it salt (4.1-H) (1 eq) and oxamic acid (1 eq) in DMF. The resulting mixture was stirred for 5 min followed by the addition of the solution of HATU (1.1 eq) in DMF. Then, the reaction mixture was stirred overnight at rt. After the completion of the reaction, monitored by LCMS, the resulting suspension was concentrated under reduced pressure. The obtained filtrate was subjected to HPLC (Waters SunFire C18 19*100 5 mkm column and H₂O-MeOH as a mobile phase) to afford pure product (Product 4.1).

### Example 19. Compound 59 (2-amino-3-methylquinolin-6-yl)((2R,5S)-5-methyl-2-(2-(1-methylpiperidin-4-yl)benzo[d]thiazol-5-yl)piperidin-1-yl)methanone

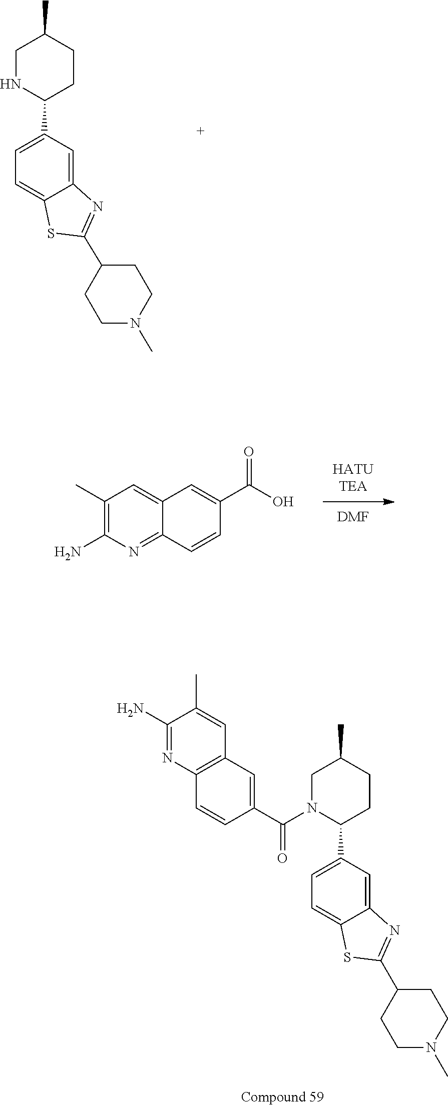

The synthesis of 5-((2R,5S)-5-methylpiperidin-2-yl)-2-(1-methylpiperidin-4-yl)benzo[d]thiazole is given by 3CCC. The synthesis of 2-amino-3-methyl-quinoline-6-carboxylic acid is given by Intermediate 1.

Prepared by general procedure scheme 4.1 step 6A. Yield: 51 mg (29.74%).

HPLC conditions: Column: XBridge C18 100*19 mm, 5 microM; 0-1-6 min 30-30-55% water-MeCN+0.1% NH₄OH; (loading pump 4 ml/min MeOH).

Compound 59: ¹H NMR (600 MHz, DMSO-d₆) δ (ppm) 0.93-1.03 (m, 3H), 1.30-1.38 (m, 1H), 1.64-1.73 (m, 1H), 1.75-1.84 (m, 3H), 2.00-2.08 (m, 4H), 2.17-2.19 (m, 6H), 2.19-2.23 (m, 1H), 2.24-2.30 (m, 1H), 2.80-2.86 (m, 2H), 3.02-3.08 (m, 1H), 3.09-3.17 (m, 1H), 3.56-3.95 (m, 1H), 5.41-5.72 (m, 1H), 6.43 (s, 2H), 7.37 (d, 1H), 7.42-7.47 (m, 2H), 7.69 (s, 1H), 7.78 (s, 1H), 7.86 (s, 1H), 8.06 (d, 1H).

LCMS(ESI): [M]⁺ m/z: calcd 513.2; found 514.2; Rt=1.905 min.

### Example 20. Compound 79 N-(6-amino-5-ethylpyridin-3-yl)-2-((5S)-5-methyl-2-(2-(1,3,3-trimethylpiperidin-4-yl)benzo[d]thiazol-5-yl)piperidin-1-yl)-2-oxoacetamide

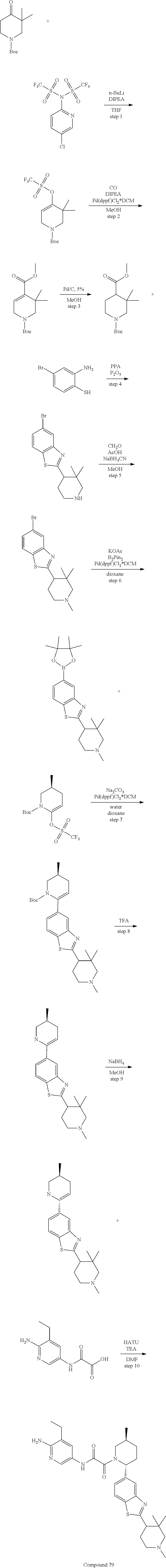

### Step 1: Synthesis of tert-butyl 5,5-dimethyl-4-(((trifluoromethyl)sulfonyl)oxy)-5,6-dihydropyridine-1(2H)-carboxylate

LDA was generated in situ by reacting a solution of DIPEA (14.47 g, 142.98 mmol, 20.15 mL) in 200 mL tetrahydrofuran with n-Butyllithium, 2.2M in hexane, packaged under Argon in resalable ChemSeal bottles (8.45 g, 131.98 mmol, 60 mL). After cooling this solution to −78° C., a solution of tert-butyl 3,3-dimethyl-4-oxo-piperidine-1-carboxylate (25 g, 109.99 mmol) in 100 ml THF was added dropwise, maintaining the reaction temperature below −70° C. After the addition was complete, the reaction mixture was stirred at −78° C. for 1 hr, and then a solution of N-(5-chloro-2-pyridyl)-1,1,1-trifluoro-N-(trifluoromethylsulfonyl)methanesulfonamide (43.19 g, 109.99 mmol) in 100 mL tetrahydrofuran was added over 5 min. The solution was allowed to warm to 0° C. and was stirred for 90 min and poured into 150 ml NH₄Cl water solution, stirred for 10 min and extracted with EtOAc (3*30 ml), combined organic layers were dried over Na₂SO₄, filtered and concentrated in vacuum. The crude product was purified by silica gel column chromatography using EtOAc:Hex (1:9, v:v) as eluent (Rt=0.5) to afford tert-butyl 3,3-dimethyl-4-(trifluoromethylsulfonyloxy)-2,6-dihydropyridine-1-carboxylate (18 g, 50.09 mmol, 45.54% yield).

LCMS(ESI): [M]⁺ m/z: calcd 359.2; found 360.2; Rt=1.650 min.

### Step 2: Synthesis of 1-tert-butyl 4-methyl 5,5-dimethyl-5,6-dihydropyridine-1,4(2H)-dicarboxylate

tert-Butyl 3,3-dimethyl-4-(trifluoromethylsulfonyloxy)-2,6-dihydropyridine-1-carboxylate (18 g, 50.09 mmol), DIPEA (7.77 g, 60.11 mmol, 10.47 mL) and Pd(dppf)Cl₂*DCM (2.05 g, 2.50 mmol) were dissolved in dry MeOH (350 mL). The reaction mixture was heated at 80° C. in high pressure vessel at 40 atm CO (48.15 g, 1.50 mol, 60.87 mL) pressure for 17 hr. Then the solvent was evaporated to dryness and the mixture was poured into 200 ml of water. The mixture was extracted with DCM (3×100 ml), organics were dried over Na₂SO₄, filtered through the thin SiO₂ pad and evaporated to dryness affording 01-tert-butyl 04-methyl 3,3-dimethyl-2,6-dihydropyridine-1,4-dicarboxylate (15 g, crude).

LCMS(ESI): [M]⁺ m/z: calcd 269.2; found 270.2; Rt=1.486 min.

### Step 3: Synthesis of 1-tert-butyl 4-methyl 3,3-dimethylpiperidine-1,4-dicarboxylate

O1-tert-butyl O4-methyl 3,3-dimethyl-2,6-dihydropyridine-1,4-dicarboxylate (15 g, 55.69 mmol) was dissolved in MeOH (200 mL) and palladium, 5% on activated carbon paste, 5R437 (4 g, 37.59 mmol) was added. The mixture was stirred under H₂ at 50° C. and 40 psi for 70 hr, filtered through celite, and evaporated to dryness to obtain 01-tert-butyl 04-methyl 3,3-dimethylpiperidine-1,4-dicarboxylate (10 g, crude).

LCMS(ESI): [M]⁺ m/z: calcd 271.2; found 272.2; Rt=1.347 min.

### Step 4: Synthesis of 5-bromo-2-(3,3-dimethylpiperidin-4-yl)benzo[d]thiazole

Prepared by general procedure scheme 4.1 step 1A. Yield: 1.8 g of crude.

LCMS(ESI): [M]⁺ m/z: calcd 325.2; found 326.2; Rt=1.044 min.

### Step 5: Synthesis of 5-bromo-2-(1,3,3-trimethylpiperidin-4-yl)benzo[d]thiazole

Formaldehyde, 37% w/w aq. soln., stab. with 7-8% MeOH (597.72 mg, 19.91 mmol, 551.91 μL) and acetic acid (683.11 mg, 11.38 mmol, 651.20 μL) were added to a stirred solution of 5-bromo-2-(3,3-dimethyl-4-piperidyl)-1,3-benzothiazole (1.85 g, 5.69 mmol) in MeOH (50 mL) at 25° C. The resulting mixture was stirred at 25° C. 0.5 hr, then sodium cyan borohydride (714.85 mg, 11.38 mmol) was added in one portion at 25° C. (foaming!). The reaction mixture was stirred at 25° C. for 18 hr, and then concentrated in vacuum. The residue was diluted with 10% aqueous sodium hydroxide solution (20 ml) and extracted with DCM (2*20 ml). The combined organic extracts were dried over sodium sulphate and concentrated in vacuum to afford crude 5-bromo-2-(1,3,3-trimethyl-4-piperidyl)-1,3-benzothiazole (1.3 g, crude).

LCMS(ESI): [M]⁺ m/z: calcd 339.2; found 340.2; Rt=1.040 min.

### Step 6: Synthesis of 5-(4,4,5,5-tetramethyl-1,3,2-dioxaborolan-2-yl)-2-(1,3,3-trimethylpiperidin-4-yl)benzo[d]thiazole

Prepared by general procedure scheme 4.1 step 2. Yield: 1.6 g of crude.

LCMS(ESI): [M]⁺ m/z: calcd 386.2; found 387.2; Rt=0.944 min.

### Step 7: Synthesis of (3S)-tert-butyl 3-methyl-6-(2-(1,3,3-trimethylpiperidin-4-yl)benzo[d]thiazol-5-yl)-3,4-dihydropyridine-1(2H)-carboxylate

Prepared by general procedure scheme 4.1 step 3. Yield: 1.2 g of crude.

LCMS(ESI): [M]⁺ m/z: calcd 455.2; found 456.2; Rt=1.316 min.

### Step 8: Synthesis of 5-((S)-5-methyl-3,4,5,6-tetrahydropyridin-2-yl)-2-(1,3,3-trimethylpiperidin-4-yl)benzo[d]thiazole

Prepared by general procedure scheme 4.1 step 4. Yield: 0.4 g of crude.

LCMS(ESI): [M]⁺ m/z: calcd 355.2; found 356.2; Rt=0.726 min.

### Step 9: Synthesis of 5-((2R,5S)-5-methylpiperidin-2-yl)-2-(1,3,3-trimethylpiperidin-4-yl)benzo[d]thiazole

Prepared by general procedure scheme 4.1 step 5. Yield: 0.5 g of crude.

LCMS(ESI): [M]⁺ m/z: calcd 357.2; found 358.2; Rt=0.634 min.

### Step 10: Synthesis of N-(6-amino-5-ethylpyridin-3-yl)-2-((5S)-5-methyl-2-(2-(1,3,3-trimethylpiperidin-4-yl)benzo[d]thiazol-5-yl)piperidin-1-yl)-2-oxoacetamide (Compound 79)

Prepared by general procedure scheme 4.1 step 6A. Yield: 23.1 mg (15.05%).

HPLC conditions: Column: XBridge C18 100*19 mm, 5 microM; 0-1-5 min 20-60% water-MeCN+0.1% NH₄OH, flow: 30 ml/min; (loading pump 4 ml/min MeOH).

Compound 79: 1H NMR (500 MHz, DMSO) δ 0.92-1.00 (m, 6H), 1.01-1.14 (m, 6H), 1.29-1.42 (m, 1H), 1.59-1.85 (m, 4H), 1.85-1.97 (m, 2H), 2.06-2.14 (m, 1H), 2.16 (s, 3H), 2.17-2.23 (m, 1H), 2.26-2.34 (m, 1H), 2.36-2.44 (m, 2H), 2.78-2.83 (m, 0.3H), 2.86-2.93 (m, 2H), 3.21-3.27 (m, 0.7H), 3.47-4.06 (m, 1H), 5.25-5.60 (m, 1H), 5.61-5.71 (m, 2H), 7.32-7.43 (m, 1H), 7.43-7.54 (m, 1H), 7.85-7.95 (m, 1H), 7.97-8.10 (m, 2H), 10.49-10.64 (m, 1H).

LCMS(ESI): [M]⁺ m/z: calcd 548.2; found 549.2; Rt=2.025 min.

### Example 21. Compound 2 and Compound 106 N-(6-amino-5-ethylpyridin-3-yl)-2-((2R,5S)-5-methyl-2-(2-(1-(pyrrolidin-1-yl)propan-2-yl)benzo[d]thiazol-5-yl)piperidin-1-yl)-2-oxoacetamide

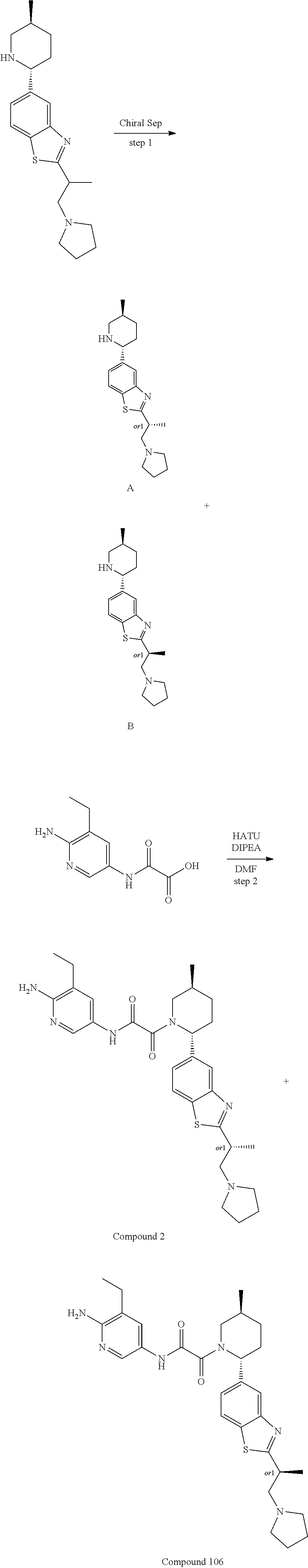

### Step 1: Chiral Separation

Racemic 5-[(2R,5S)-5-methyl-2-piperidyl]-2-[1-methyl-2-pyrrolidin-1-yl-ethyl]-1,3-benzothiazole (0.5 g, 1.46 mmol) (purity=72.4%) was purified with reverse phase HPLC (SYSTEM 40-90% 0-5 min H₂O/MeOH/0.1% NH₄OH, flow: 30 ml/min (loading pump 4 ml/min MeOH); column: YMC Triart C18 100×20 mm, 5 um). The obtained racemate (m=0.15 g., purity=97.4%) was chiral separated (Column: Chiralcel OD-H (250*20 mm, 5 mkm); Hexane-IPA-MeOH, 90-5-5; 17 ml/min) to obtain 5-[(2R,5S)-5-methyl-2-piperidyl]-2-[(1R)-1-methyl-2-pyrrolidin-1-yl-ethyl]-1,3-benzothiazole (0.04914 g, 143.05 mol, 9.83% yield) (RT=9.4 min) and 5-[(2R,5S)-5-methyl-2-piperidyl]-2-[(1S)-1-methyl-2-pyrrolidin-1-yl-ethyl]-1,3-benzothiazole (47.06 mg, 136.99 mol, 9.41% yield) (RT=11.31 min).

Rel Time for A in analytical conditions (column: OD-H, Hexane-IPA-MeOH, 90-5-5, 0.6 ml/min as mobile phase) 9.38 min and for B 10.66 min.

### A: Retention Time: 9.38 Min

LCMS(ESI): [M]⁺ m/z: calcd 343.2; found 344.2; Rt=1.708 min.

### B: Retention Time: 10.66 Min

LCMS(ESI): [M]⁺ m/z: calcd 343.2; found 344.2; Rt=1.712 min.

### Step 2: Synthesis of N-(6-amino-5-ethylpyridin-3-yl)-2-((2R,5S)-5-methyl-2-(2-(1-(pyrrolidin-1-yl)propan-2-yl)benzo[d]thiazol-5-yl)piperidin-1-yl)-2-oxoacetamide (Compound 2 and Compound 106)

Prepared by general procedure scheme 4.1 step 6B. Yield: 42.6 mg (55.69%) for Compound 2 and 37.4 mg (50.98%) for Compound 106.

HPLC conditions: Column: YMC Triart C18 100*20 mm, 5 microM; 0-6 min 45-90% water-MeOH+0.1% NH₄OH, flow: 30 ml/min; (loading pump 4 ml/min MeOH).

Compound 2: 1H NMR (600 MHz, dmso) δ 1.01-1.14 (m, 6H), 1.31-1.40 (m, 4H), 1.64-1.73 (m, 5H), 1.81-1.92 (m, 1H), 2.04-2.22 (m, 1H), 2.23-2.36 (m, 2H), 2.38-2.41 (m, 1H), 2.43-2.46 (m, 2H), 2.52-2.56 (m, 2H), 2.61-2.66 (m, 1H), 2.77-3.27 (m, 2H), 3.40-3.45 (m, 1H), 3.46-4.10 (m, 1H), 5.26-5.74 (m, 3H), 7.30-7.42 (m, 1H), 7.44-7.56 (m, 1H), 7.84-7.92 (m, 1H), 7.98-8.10 (m, 2H), 10.42 (s, 1H).

LCMS(ESI): [M+1]⁺ m/z: calcd 534.2; found 535.2; Rt=2.269 min.

Compound 106: H NMR (600 MHz, dmso) δ 1.01-1.15 (m, 6H), 1.31-1.41 (m, 4H), 1.63-1.73 (m, 5H), 1.82-1.92 (m, 1H), 2.03-2.36 (m, 3H), 2.39-2.46 (m, 3H), 2.51-2.55 (m, 2H), 2.61-2.67 (m, 1H), 2.76-3.25 (m, 2H), 3.39-3.44 (m, 1H), 3.47-4.08 (m, 1H), 5.24-5.73 (m, 3H), 7.30-7.42 (m, 1H), 7.43-7.53 (m, 1H), 7.83-7.93 (m, 1H), 7.98-8.09 (m, 2H), 10.17 (s, 1H).

LCMS(ESI): [M+1]⁺ m/z: calcd 534.2; found 535.2; Rt=2.271 min.

### Example 22. Compound 13 N-(6-amino-5-ethylpyridin-3-yl)-2-((2R,5S)-5-methyl-2-(2-(1,5,5-trimethylpyrrolidin-3-yl)benzo[d]thiazol-5-yl)piperidin-1-yl)-2-oxoacetamide

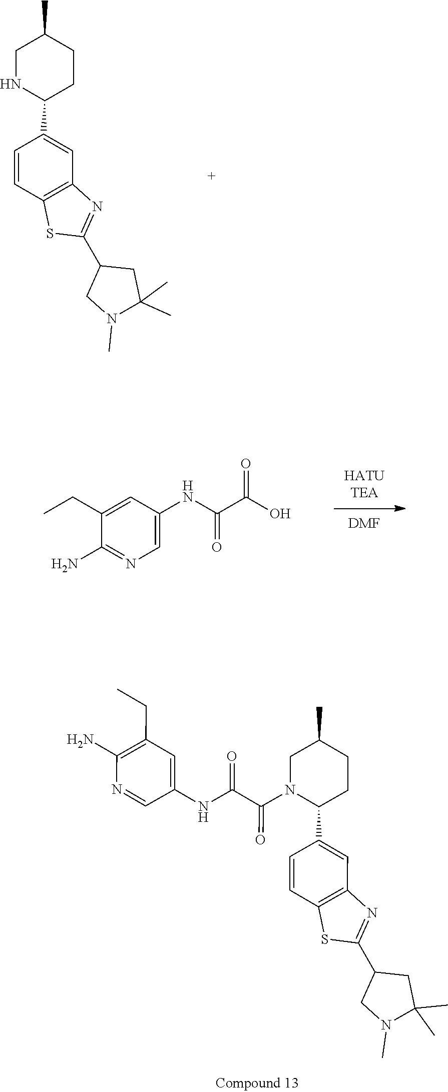

The synthesis of 5-((2R,5S)-5-methylpiperidin-2-yl)-2-(1,5,5-trimethylpyrrolidin-3-yl)benzo[d]thiazole is given by 3FFF.

Prepared by general procedure scheme 4.1 step 6A. Yield: 400 mg (57.11%).

HPLC conditions: Column: YMC Triart C18 100*20 mm, 5 microM; 0-1-6 min 60-60-75% water-MeOH+0.1% NH₄OH; (loading pump 4 ml/min MeOH).

Compound 13: ¹H NMR (600 MHz, DMSO-d₆) δ (ppm) 0.98-1.14 (m, 12H), 1.27-1.43 (m, 1H), 1.63-1.75 (m, 1H), 1.81-1.93 (m, 1H), 1.94-2.15 (m, 2H), 2.17 (s, 3H), 2.19-2.36 (m, 3H), 2.39-2.42 (m, 1H), 2.72-3.12 (m, 3H), 3.46-4.06 (m, 2H), 5.21-5.72 (m, 3H), 7.29-7.41 (m, 1H), 7.41-7.53 (m, 1H), 7.79-7.91 (m, 1H), 7.94-8.07 (m, 2H), 10.47-10.60 (m, 1H).

LCMS(ESI): [M]⁺ m/z: calcd 534.2; found 535.2; Rt=2.111 min.

### Example 23. Compound 55 of N-(6-amino-5-ethylpyridin-3-yl)-2-((2R,5S)-2-(2-((S)-2-(dimethylamino)propyl)benzo[d]thiazol-5-yl)-5-methylpiperidin-1-yl)-2-oxoacetamide

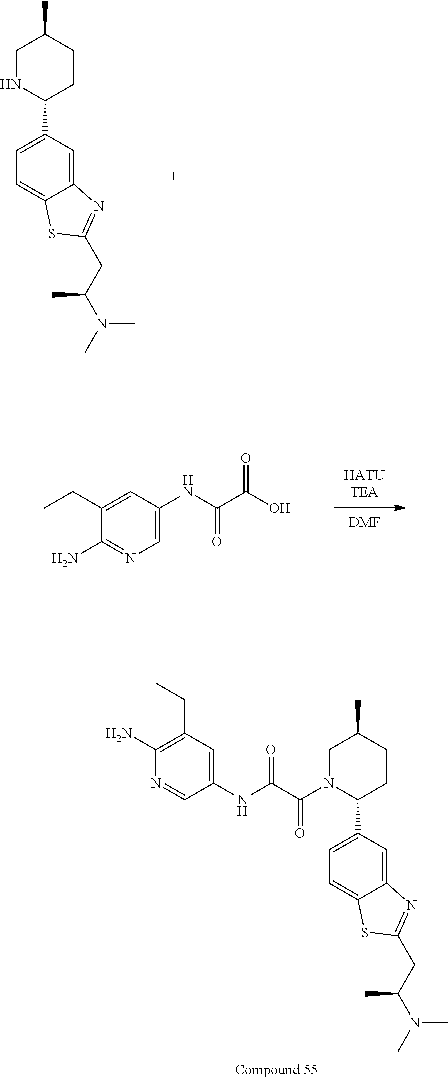

Synthesis of (S)—N,N-dimethyl-1-(5-((2R,5S)-5-methylpiperidin-2-yl)benzo[d]thiazol-2-yl)propan-2-amine is given by 3GGG.

Prepared by general procedure scheme 4.1 step 6B. Yield: 18 mg (5.62%).

HPLC conditions: Column: XBridge C18 100*19 mm, 5 microM; 0-5 min 30-80% water-MeOH+0.1% NH₄OH; (loading pump 4 ml/min MeOH).

Compound 55: ¹H NMR (600 MHz, DMSO-d₆) δ (ppm) 0.97-1.00 (m, 3H), 1.01-1.05 (m, 3H), 1.06-1.15 (m, 3H), 1.30-1.45 (m, 1H), 1.63-1.74 (m, 1H), 1.80-1.92 (m, 1H), 1.97-2.18 (m, 1H), 2.21 (s, 6H), 2.25-2.33 (m, 1H), 2.36-2.43 (m, 2H), 2.77-2.81 (m, 0.3H), 2.97-3.07 (m, 2H), 3.14-3.26 (m, 1.7H), 3.49-4.09 (m, 1H), 5.23-5.61 (m, 1H), 5.61-5.76 (m, 2H), 7.29-7.41 (m, 1H), 7.41-7.55 (m, 1H), 7.79-7.89 (m, 1H), 7.97-8.09 (m, 2H), 10.45-10.59 (m, 1H).

LCMS(ESI): [M]⁺ m/z: calcd 508.2; found 509.2; Rt=1.093 min.

### Example 24. Compound 41 N-(6-amino-5-ethylpyridin-3-yl)-2-((2R,5S)-5-methyl-2-(2-((1-methylpiperidin-4-yl)methyl)benzo[d]thiazol-5-yl)piperidin-1-yl)-2-oxoacetamide

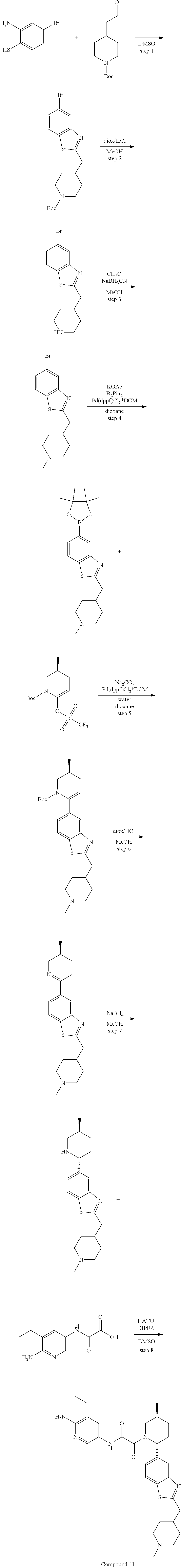

### Step 1: Synthesis of tert-butyl 4-((5-bromobenzo[d]thiazol-2-yl)methyl)piperidine-1-carboxylate)

Prepared by general procedure scheme 4.1 step 1B. Yield: 2.1 g (38.68%).

LCMS(ESI): [M]⁺ m/z: calcd 411.2; found 412.2; Rt=1.128 min.

### Step 2: Synthesis of 5-bromo-2-(piperidin-4-ylmethyl)benzo[d]thiazole

tert-Butyl 4-[(5-bromo-1,3-benzothiazol-2-yl)methyl]piperidine-1-carboxylate (2.1 g, 5.11 mmol) was dissolved in mixture of MeOH (16.36 mL) and HCl in dioxane (102.10 mmol), then stirred for 1 hr. The reaction mixture was concentrated in vacuum, the residue was treated with aq. solution of NaHCO₃ and desired product was extracted with DCM (2*40 ml), dried over Na₂SO₄, evaporated in vacuum to give 5-bromo-2-(4-piperidylmethyl)-1,3-benzothiazole (1.6 g, 5.14 mmol, 100.70% yield).

LCMS(ESI): [M]⁺ m/z: calcd 311.2; found 312.2; Rt=0.966 min.

### Step 3: Synthesis of 5-bromo-2-((1-methylpiperidin-4-yl)methyl)benzo[d]thiazole

Sodium cyan borohydride (1.62 g, 25.70 mmol) was added to a mixture of 5-bromo-2-(4-piperidylmethyl)-1,3-benzothiazole (1.6 g, 5.14 mmol) and formalin (2.09 g, 25.70 mmol, 1.93 mL, 37% purity) in MeOH (20 mL) and stirred overnight, then concentrated. The residue was treated with aq. solution of NaHCO₃ and desired product was extracted with DCM (2*30 ml), dried over Na₂SO₄ and concentrated in vacuum to give 5-bromo-2-[(1-methyl-4-piperidyl)methyl]-1,3-benzothiazole (1.5 g, 4.61 mmol, 89.71% yield).

LCMS(ESI): [M]⁺ m/z: calcd 325.2; found 326.2; Rt=1.012 min.

### Step 4: Synthesis of 2-((1-methylpiperidin-4-yl)methyl)-5-(4,4,5,5-tetramethyl-1,3,2-dioxaborolan-2-yl)benzo[d]thiazole

Prepared by general procedure scheme 4.1 step 2. Yield: 1.8 g of crude.

LCMS(ESI): [M]⁺ m/z: calcd 372.2; found 373.2; Rt=1.139 min.

### Step 5: Synthesis of (S)-tert-butyl 3-methyl-6-(2-((1-methylpiperidin-4-yl)methyl)benzo[d]thiazol-5-yl)-3,4-dihydropyridine-1(2H)-carboxylate

Prepared by general procedure scheme 4.1 step 3. Yield: 2.1 g of crude.

LCMS(ESI): [M]⁺ m/z: calcd 441.2; found 442.2; Rt=1.231 min.

### Step 6: Synthesis of (S)-5-(5-methyl-3,4,5,6-tetrahydropyridin-2-yl)-2-((1-methylpiperidin-4-yl)methyl)benzo[d]thiazole

tert-Butyl (3S)-3-methyl-6-[2-[(1-methyl-4-piperidyl)methyl]-1,3-benzothiazol-5-yl]-3,4-dihydro-2H-pyridine-1-carboxylate (1.2 g, 2.72 mmol) was dissolved in MeOH (50 mL) and diox/HCl (54.34 mmol, 30 mL) was added thereto. Then it was stirred at rt for 2 hr. The reaction mixture was evaporated to afford 2-[(1-methyl-4-piperidyl)methyl]-5-[(3S)-3-methyl-2,3,4,5-tetrahydropyridin-6-yl]-1,3-benzothiazole (0.9 g, 2.64 mmol, 96.99% yield).

LCMS(ESI): [M]⁺ m/z: calcd 341.2; found 342.2; Rt=0.694 min.

### Step 7: Synthesis of 5-((2R,5S)-5-methylpiperidin-2-yl)-2-((1-methylpiperidin-4-yl)methyl)benzo[d]thiazole

Prepared by general procedure scheme 4.1 step 5. Yield: 0.98 g of crude.

LCMS(ESI): [M]⁺ m/z: calcd 343.2; found 344.2; Rt=0.637 min.

### Step 8: Synthesis of N-(6-amino-5-ethylpyridin-3-yl)-2-((2R,5S)-5-methyl-2-(2-((1-methylpiperidin-4-yl)methyl)benzo[d]thiazol-5-yl)piperidin-1-yl)-2-oxoacetamide (Compound 41)

Prepared by general procedure scheme 4.1 step 6B. Yield: 13.7 mg (8.1%).

### HPLC Conditions:

First run: Column: SunFire 100*19 mm, 5 microM; 2-10 min 10-40% MeOH+NH₃, flow: 30 ml/min; (loading pump 4 ml/min MeOH+NH₃).

Second run: Column: SunFire 100*19 mm, 5 microM; 2-10 min 10-40% MeCN+FA, flow: 30 ml/min; (loading pump 4 ml/min MeCN).

Compound 41: 1H NMR (600 MHz, dmso) δ 1.00-1.05 (m, 3H), 1.06-1.16 (m, 3H), 1.27-1.42 (m, 3H), 1.60-1.72 (m, 3H), 1.75-1.89 (m, 2H), 1.92-1.99 (m, 2H), 2.05-2.14 (m, 1H), 2.17 (s, 3H), 2.22-2.36 (m, 2H), 2.37-2.45 (m, 2H), 2.76-2.80 (m, 2H), 3.00-3.03 (m, 2H), 3.49-3.50 (m, 0.7H), 4.03-4.06 (m, 0.3H), 5.25-5.60 (m, 1H), 5.61-5.71 (m, 2H), 7.31-7.42 (m, 1H), 7.42-7.54 (m, 1H), 7.82-7.92 (m, 1H), 7.98-8.09 (m, 2H), 10.49-10.65 (m, 1H).

LCMS(ESI): [M]⁺ m/z: calcd 534.2; found 535.2; Rt=2.260 min.

### Example 25. Compound 1 and Compound 34 N-(6-amino-5-(oxetan-3-yl)pyridin-3-yl)-2-((2R,5S)-5-methyl-2-(2-(1,2,2-trimethylpiperidin-4-yl)benzo[d]thiazol-5-yl)piperidin-1-yl)-2-oxoacetamide

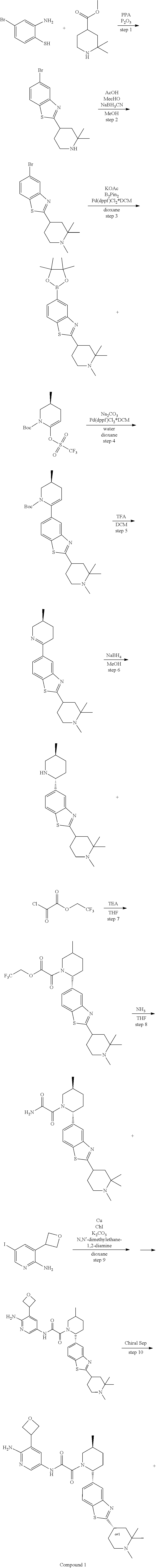

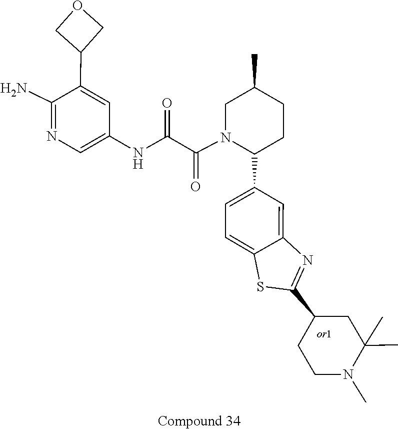

### Step 1: Synthesis of 5-bromo-2-(2,2-dimethylpiperidin-4-yl)benzo[d]thiazole

Prepared by general procedure scheme 4.1 step IA. Yield: 9.5 g (59.61%).

¹H NMR (500 MHz, CDCl₃) δ (ppm) 1.21 (s, 3H), 1.25 (s, 3H), 1.63 (m, 3H), 2.12 (m, 2H), 3.03 (m, 2H), 3.43 (m, 1H), 7.46 (d, 1H), 7.70 (d, 1H), 8.11 (s, 1H).

### Step 2: Synthesis of 5-bromo-2-(1,2,2-trimethylpiperidin-4-yl)benzo[d]thiazole

Formaldehyde, 37% w/w aq. soln., stab. with 7-8% MeOH (2.63 g, 87.62 mmol, 2.43 mL) and acetic acid (5.26 g, 87.62 mmol, 5.02 mL) were added to the solution of 5-bromo-2-(2,2-dimethyl-4-piperidyl)-1,3-benzothiazole (9.5 g, 29.21 mmol) in MeOH (27.26 mL). Resulting mixture was stirred at 0° C. for 1 hr before sodium cyan borohydride (3.67 g, 58.41 mmol) was added thereto. After that, stirring was continued for 56 hr. Then, solvent was removed under reduced pressure and residue was partitioned between 10% aq. K₂CO₃ solution (50 ml) and DCM (80 ml). Organic layer was separated, dried over solid K₂CO₃ and concentrated under reduced pressure, leaving 5-bromo-2-(1,2,2-trimethyl-4-piperidyl)-1,3-benzothiazole (9 g, 26.53 mmol, 90.82% yield).

¹H NMR (400 MHz, CDCl₃) δ (ppm) 1.19 (s, 3H), 1.32 (s, 3H), 2.08 (m, 4H), 2.46 (s, 3H), 2.82 (m, 1H), 3.05 (m, 1H), 3.41 (m, 1H), 7.45 (d, 1H), 7.70 (d, 1H), 8.0808 (s, 1H).

### Step 3: Synthesis of 5-(4,4,5,5-tetramethyl-1,3,2-dioxaborolan-2-yl)-2-(1,2,2-trimethylpiperidin-4-yl)benzo[d]thiazole

Prepared by general procedure scheme 4.1 step 2. Yield: 12 g of crude.

LCMS(ESI): [M]⁺ m/z: calcd 386.2; found 387.2; Rt=1.063 min.

### Step 4: Synthesis of (3S)-tert-butyl 3-methyl-6-(2-(1,2,2-trimethylpiperidin-4-yl)benzo[d]thiazol-5-yl)-3,4-dihydropyridine-1(2H)-carboxylate

Prepared by general procedure scheme 4.1 step 3. Yield: 11 g of crude.

LCMS(ESI): [M]⁺ m/z: calcd 455.2; found 456.2; Rt=3.365 min.

### Step 5: Synthesis of 5-((S)-5-methyl-3,4,5,6-tetrahydropyridin-2-yl)-2-(1,2,2-trimethylpiperidin-4-yl)benzo[d]thiazole

Prepared by general procedure scheme 4.1 step 4. Yield: 8.5 g of crude.

LCMS(ESI): [M]⁺ m/z: calcd 355.2; found 356.2; Rt=0.510 min.

### Step 6: Synthesis of 5-((2R,5S)-5-methylpiperidin-2-yl)-2-(1,2,2-trimethylpiperidin-4-yl)benzo[d]thiazole

Prepared by general procedure scheme 4.1 step 5. Yield: 7 g (81.89%).

LCMS(ESI): [M]⁺ m/z: calcd 357.2; found 358.2; Rt=0.773 min.

### Step 7: Synthesis of 2,2,2-trifluoroethyl 2-((2R,5S)-5-methyl-2-(2-(1,2,2-trimethylpiperidin-4-yl)benzo[d]thiazol-5-yl)piperidin-1-yl)-2-oxoacetate

Prepared by general procedure scheme 4.2 step 1. Yield: 0.8 g of crude.

LCMS(ESI): [M]⁺ m/z: calcd 511.2; found 512.2; Rt=1.199 min.

### Step 8: Synthesis of 2-((2R,5S)-5-methyl-2-(2-(1,2,2-trimethylpiperidin-4-yl)benzo[d]thiazol-5-yl)piperidin-1-yl)-2-oxoacetamide

Prepared by general procedure scheme 4.2 step 2. Yield: 0.6 g of crude.

LCMS(ESI): [M]⁺ m/z: calcd 428.2; found 429.2; Rt=1.984 min.

### Step 9: Synthesis of N-(6-amino-5-(oxetan-3-yl)pyridin-3-yl)-2-((2R,5S)-5-methyl-2-(2-(1,2,2-trimethylpiperidin-4-yl)benzo[d]thiazol-5-yl)piperidin-1-yl)-2-oxoacetamide

Prepared by general procedure scheme 4.2 step 3A. Yield: 174 mg (13.01%).

HPLC conditions: Column: XBridge BEH C18 100*19 mm, 5 microM; 0-1-6 min 30-30-50% water-MeCN+0.1% NH₄OH, flow: 30 ml/min; (loading pump 4 ml/min MeCN).

LCMS(ESI): [M]⁺ m/z: calcd 576.2; found 577.2; Rt=2.337 min.

### Step 10: Chiral Separation (Compound 1 and Compound 34)

Racemic N-[6-amino-5-(oxetan-3-yl)-3-pyridyl]-2-oxo-2-[(2R,5S)-5-methyl-2-[2-(1,2,2-trimethyl-4-piperidyl)-2,3-dihydro-1,3-benzothiazol-5-yl]-1-piperidyl]acetamide (0.0174 g, 30.06 mol) was chiral separated (Column: Chiralpak IA-III (250-20 mm-5 m); Mobile phase: Hexane-IPA-MeOH, 50-20-30 Flow Rate: 12 mL/min) to obtain N-[6-amino-5-(oxetan-3-yl)-3-pyridyl]-2-oxo-2-[(2R,5S)-5-methyl-2-[2-[(4S)-1,2,2-trimethyl-4-piperidyl]-2,3-dihydro-1,3-benzothiazol-5-yl]-1-piperidyl]acetamide (0.00607 g, 10.49 mol, 34.89% yield) and N-[6-amino-5-(oxetan-3-yl)-3-pyridyl]-2-oxo-2-[(2R,5S)-5-methyl-2-[2-[(4R)-1,2,2-trimethyl-4-piperidyl]-2,3-dihydro-1,3-benzothiazol-5-yl]-1-piperidyl]acetamide (0.05775 g, 124.60 umol, 50.22% yield).

Rel Time for Compound 1 in analytical conditions (column: IA, MeOH-IPA, 50-50, 0.6 ml/min as mobile phase) 53.75 min and for Compound 34 70.55 min.

### Compound 1: Retention Time: 53.75 Min

LCMS(ESI): [M]⁺ m/z: calcd 576.2; found 577.2; Rt=0.898 min.

### Compound 34: Retention Time: 70.55 Min

LCMS(ESI): [M]⁺ m/z: calcd 576.2; found 577.2; Rt=0.897 min.

### Example 26. Compound 3 2-methoxy-5-(2-((5S)-5-methyl-2-(2-(1,3,3-trimethylpiperidin-4-yl)benzo[d]thiazol-5-yl)piperidin-1-yl)-2-oxoacetamido)nicotinamide

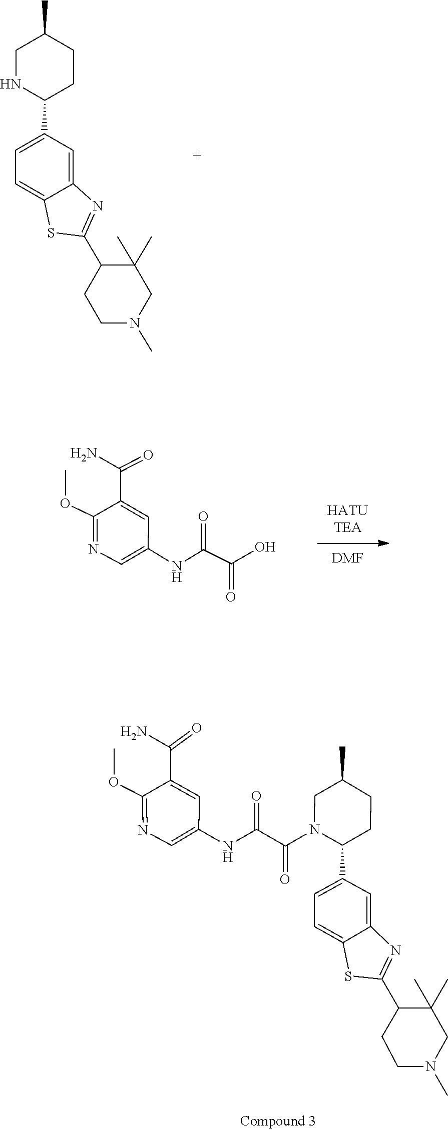

The synthesis of 5-((2R,5S)-5-methylpiperidin-2-yl)-2-(1,3,3-trimethylpiperidin-4-yl)benzo[d]thiazole is given by Example 5 (Compound 79), step 1-9.

Prepared by general procedure scheme 4.1 step 6A. Yield: 18.8 mg (11.62%).

HPLC conditions: Column: XBridge C18 100*19 mm, 5 microM; 0-1-5 min 25-65% water-MeCN+0.1% NH₄OH, flow: 30 ml/min; (loading pump 4 ml/min MeOH).

Compound 3: ¹H NMR (600 MHz, DMSO-d₆) δ (ppm) 0.91-0.95 (m, 3H), 0.97-1.00 (m, 3H), 1.00-1.08 (m, 3H), 1.29-1.42 (m, 1H), 1.70-1.82 (m, 3H), 1.83-1.94 (m, 2H), 2.06-2.13 (m, 1H), 2.15 (s, 3H), 2.16-2.23 (m, 1H), 2.28-2.35 (m, 1H), 2.37-2.45 (m, 1H), 2.84-3.24 (m, 3H), 3.38-3.56 (m, 0.7H), 3.88-3.98 (m, 3H), 4.00-4.07 (m, 0.3H), 5.15-5.83 (m, 1H), 7.32-7.47 (m, 1H), 7.65-7.80 (m, 2H), 7.88-7.96 (m, 1H), 8.00-8.12 (m, 1H), 8.36-8.62 (m, 2H), 10.90-11.30 (m, 1H).

LCMS(ESI): [M]⁺ m/z: calcd 578.2; found 579.2; Rt=2.679 min.

### Example 27. Compound 56 and Compound 27 N-(6-amino-5-ethylpyridin-3-yl)-2-((2R,5S)-5-methyl-2-(2-(1,5,5-trimethylpyrrolidin-3-yl)benzo[d]thiazol-5-yl)piperidin-1-yl)-2-oxoacetamide

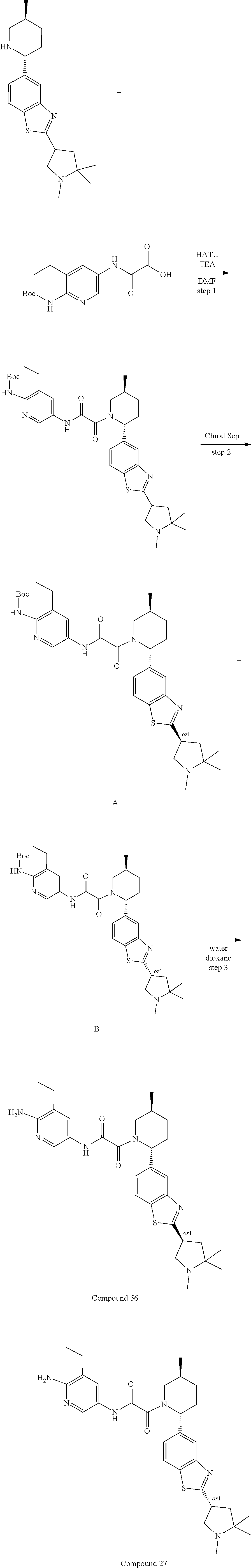

The synthesis of 5-((2R,5S)-5-methylpiperidin-2-yl)-2-(1,5,5-trimethylpyrrolidin-3-yl)benzo[d]thiazole is given by 3FFF.

### Step 1: Synthesis of tert-butyl (3-ethyl-5-(2-((2R,5S)-5-methyl-2-(2-(1,5,5-trimethylpyrrolidin-3-yl)benzo[d]thiazol-5-yl)piperidin-1-yl)-2-oxoacetamido)pyridin-2-yl)carbamate

Prepared by general procedure scheme 4.1 step 6A. Yield: 121 mg (32.74%).

HPLC conditions: Column: YMC Triart C18 100*20 mm, 5 microM; 0-1-6 min 50-100% water-MeOH+0.1% NH₄OH; (loading pump 4 ml/min MeOH).

LCMS(ESI): [M]⁺ m/z: calcd 634.2; found 635.2; Rt=2.896 min.

### Step 2: Chiral Separation

Racemic tert-butyl N-[3-ethyl-5-[[2-oxo-2-[(2R,5S)-5-methyl-2-[2-(1,5,5-trimethylpyrrolidin-3-yl)-1,3-benzothiazol-5-yl]-1-piperidyl]acetyl]amino]-2-pyridyl]carbamate (121 mg, 190.60 μmol) was submitted to preparative chiral HPLC (Column: Chiralpak IA-II (250*20 mm, 5 mkm); Mobile phase: Hexane-IPA-MeOH 60-20-20 Flow Rate: 12 mL/min) to afford crude 1 st enantiomer (RT=34.166 min., 37 mg), crude 2nd enantiomer (RT=41.448 min., 44 mg) and racemate (22 mg). Crude 1st enantiomer was purified by preparative chiral HPLC (Column: Chiralpak IJ-I (250*20 mm, 5 mkm); Mobile phase: Hexane-IPA-MeOH, 70-15-15; Flow rate: 12 ml/min) to afford tert-butyl N-[3-ethyl-5-[[2-oxo-2-[(2R,5S)-5-methyl-2-[2-[(35)-1,5,5-trimethylpyrrolidin-3-yl]-1,3-benzothiazol-5-yl]-1-piperidyl]acetyl]amino]-2-pyridyl]carbamate (21 mg, 33.08 μmol, 17.36% yield) (RT=8.295 min.) as light-yellow gum. Crude 2nd enantiomer was purified by preparative chiral HPLC (Column: Chiralpak IA-II (250*20 mm, 5 mkm); Mobile phase: Hexane-IPA-MeOH, 60-20-20; Flow rate: 12 ml/min) to afford tert-butyl N-[3-ethyl-5-[[2-oxo-2-[(2R,5S)-5-methyl-2-[2-[(3R)-1,5,5-trimethylpyrrolidin-3-yl]-1,3-benzothiazol-5-yl]-1-piperidyl]acetyl]amino]-2-pyridyl]carbamate (24 mg, 37.81 μmol, 19.83% yield) (RT=38.022 min.) as light-yellow gum.

Rel Time for A in analytical conditions (column: IA, Hexane-IPA-MeOH, 60-20-20, 0.6 ml/min as mobile phase) 29.59 min and for B 36.68 min.

A: Retention time: 29.59 min. LCMS(ESI): [M]⁺ m/z: calcd 634.2; found 635.2; Rt=1.210 min.

B: Retention time: 36.68 min. LCMS(ESI): [M]⁺ m/z: calcd 634.2; found 635.2; Rt=1.212 min.

### Step 3: Synthesis of N-(6-amino-5-ethylpyridin-3-yl)-2-((2R,5S)-5-methyl-2-(2-(1,5,5-trimethylpyrrolidin-3-yl)benzo[d]thiazol-5-yl)piperidin-1-yl)-2-oxoacetamide (Compound 56 and Compound 27)

A solutions of tert-butyl N-[3-ethyl-5-[[2-oxo-2-[(2R,5S)-5-methyl-2-[2-[(35)-1,5,5-trimethylpyrrolidin-3-yl]-1,3-benzothiazol-5-yl]-1-piperidyl]acetyl]amino]-2-pyridyl]carbamate (21 mg, 33.08 μmol) and tert-butyl N-[3-ethyl-5-[[2-oxo-2-[(2R,5S)-5-methyl-2-[2-[(3R)-1,5,5-trimethylpyrrolidin-3-yl]-1,3-benzothiazol-5-yl]-1-piperidyl]acetyl]amino]-2-pyridyl]carbamate (24.00 mg, 37.81 mol) in a mixture of 1.4-dioxane (0.7 mL) and water (0.7 mL) were stirred at 90° C. for 24 hr, then cooled down and concentrated in vacuum. The residues were lyophilized to afford Compound 56 N-(6-amino-5-ethyl-3-pyridyl)-2-oxo-2-[(2R,5S)-5-methyl-2-[2-[(3S)-1,5,5-trimethylpyrrolidin-3-yl]-1,3-benzothiazol-5-yl]-1-piperidyl]acetamide (15 mg, 28.05 mol, 84.80% yield) and Compound 27 N-(6-amino-5-ethyl-3-pyridyl)-2-oxo-2-[(2R,5S)-5-methyl-2-[2-[(3R)-1,5,5-trimethylpyrrolidin-3-yl]-1,3-benzothiazol-5-yl]-1-piperidyl]acetamide (15 mg, 28.05 mol, 74.20% yield) as light-yellow solids.

Compound 56: ¹H NMR (600 MHz, DMSO-d₆) δ (ppm) 0.96-1.15 (m, 12H), 1.32-1.40 (m, 1H), 1.65-1.76 (m, 1H), 1.82-1.92 (m, 1H), 1.96-2.01 (m, 1H), 2.04-2.21 (m, 4H), 2.22-2.35 (m, 2H), 2.39-2.42 (m, 1H), 2.77-3.12 (m, 3H), 3.45-4.08 (m, 3H), 5.24-5.72 (m, 3H), 7.31-7.42 (m, 1H), 7.42-7.53 (m, 1H), 7.80-7.91 (m, 1H), 7.97-8.08 (m, 2H), 10.50-10.62 (m, 1H).

LCMS(ESI): [M]⁺ m/z: calcd 534.2; found 535.2; Rt=1.183 min.

Compound 27: ¹H NMR (600 MHz, DMSO-d₆) δ (ppm) 0.99-1.14 (m, 12H), 1.32-1.40 (m, 1H), 1.66-1.73 (m, 1H), 1.83-1.91 (m, 1H), 1.98 (dd, 1H), 2.05-2.20 (m, 4H), 2.20-2.35 (m, 3H), 2.39-2.42 (m, 1H), 2.77-3.11 (m, 3H), 3.45-4.09 (m, 2H), 5.23-5.76 (m, 3H), 7.30-7.42 (m, 1H), 7.42-7.53 (m, 1H), 7.81-7.88 (m, 1H), 7.98-8.09 (m, 2H), 10.51-10.64 (m, 1H).

LCMS(ESI): [M]⁺ m/z: calcd 534.2; found 535.2; Rt=1.183 min.

### Example 28. Compound 118, Compound 75 and Compound 23 N-(6-amino-5-ethylpyridin-3-yl)-2-((2R,5S)-2-(2-(1,5-dimethylpiperidin-3-yl)benzo[d]thiazol-5-yl)-5-methylpiperidin-1-yl)-2-oxoacetamide

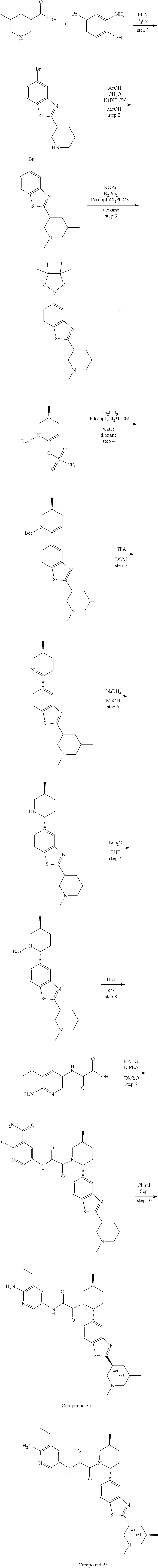

### Step 1: Synthesis of 5-bromo-2-(5-methylpiperidin-3-yl)benzo[d]thiazole

Prepared by general procedure scheme 4.1 step 1A. Yield: 3.1 g of crude.

LCMS(ESI): [M]⁺ m/z: calcd 311.2; found 312.2; Rt=2.570 min.

### Step 2: Synthesis of 5-bromo-2-(1,5-dimethylpiperidin-3-yl)benzo[d]

Formaldehyde (808.28 mg, 9.96 mmol, 746.33 μL) (37% solution in water) was added to a stirred solution of 5-bromo-2-(5-methyl-3-piperidyl)-1,3-benzothiazole (3.1 g, 9.96 mmol) and acetic acid (1.20 g, 19.92 mmol, 1.14 mL) in MeOH (28.86 mL), resulting mixture stirred at 25° C. for 1 hr, sodium cyan borohydride (625.92 mg, 9.96 mmol) added in 1 portion and stirred at 25° C. for 18 hr. Reaction mixture concentrated, mixed with NaOH (20% in water, 10 mL), extracted with MTBE (2×20 mL), combined organic layer dried over Na₂SO₄, concentrated to give crude 5-bromo-2-(1,5-dimethyl-3-piperidyl)-1,3-benzothiazole (1.05 g, 3.23 mmol, 32.41% yield).

LCMS(ESI): [M]⁺ m/z: calcd 325.2; found 326.2; Rt=1.038 min.

### Step 3: Synthesis of 2-(1,5-dimethylpiperidin-3-yl)-5-(4,4,5,5-tetramethyl-1,3,2-dioxaborolan-2-yl)benzo[d]thiazole

Prepared by general procedure scheme 4.1 step 2. Yield: 1.1 g (96.1%).

LCMS(ESI): [M]⁺ m/z: calcd 372.2; found 373.2; Rt=2.772 min.

### Step 4: Synthesis of (3S)-tert-butyl 6-(2-(1,5-dimethylpiperidin-3-yl)benzo[d]thiazol-5-yl)-3-methyl-3,4-dihydropyridine-1(2H)-carboxylate

Prepared by general procedure scheme 4.1 step 3. Yield: 1.2 g of crude.

LCMS(ESI): [M]⁺ m/z: calcd 441.2; found 442.2; Rt=3.100 min.

### Step 5: Synthesis of 2-(1,5-dimethylpiperidin-3-yl)-5-((S)-5-methyl-3,4,5,6-tetrahydropyridin-2-yl)benzo[d]thiazole

Prepared by general procedure scheme 4.1 step 4. Yield: 0.6 g of crude.

LCMS(ESI): [M]⁺ m/z: calcd 341.2; found 342.2; Rt=1.535 min.

### Step 6: Synthesis of 2-(1,5-dimethylpiperidin-3-yl)-5-((2R,5S)-5-methylpiperidin-2-yl)benzo[d]thiazole

Prepared by general procedure scheme 4.1 step 5. Yield: 0.5 g of crude.

LCMS(ESI): [M]⁺ m/z: calcd 343.2; found 344.2; Rt=0.673 min.

### Step 7: Synthesis of (2R,5S)-tert-butyl 2-(2-(1,5-dimethylpiperidin-3-yl)benzo[d]thiazol-5-yl)-5-methylpiperidine-1-carboxylate

To a solution of 2-(1,5-dimethylpiperidin-3-yl)-5-((2R,5S)-5-methylpiperidin-2-yl)benzo[d]thiazole (0.55 g, 1.60 mmol) in THF (2.67 mL), di-tert-butyl dicarbonate (349.43 mg, 1.60 mmol, 367.43 μL) was added. The resulting mixture was stirred at 25° C. for 8 hr and purified by HPLC (column: XBridge BEH C18 5 μm 130A; mobile phase: 70-95% 0-5 min H₂O/MeOH/0.1% NH₄OH, flow rate: 30 ml/min) to obtain (2R,5S)-tert-butyl 2-(2-(1,5-dimethylpiperidin-3-yl)benzo[d]thiazol-5-yl)-5-methylpiperidine-1-carboxylate (0.43 g, 969.25 mol, 60.54% yield) (two diastereomers were separated).

LCMS(ESI): [M]⁺ m/z: calcd 443.2; found 444.2; Rt=3.178 min.

### Step 8: Synthesis of 2-(1,5-dimethylpiperidin-3-yl)-5-((2R,5S)-5-methylpiperidin-2-yl)benzo[d]thiazole

TFA (539.72 mg, 4.73 mmol, 364.67 μL) was added to a mixture of tert-butyl (2R,5S)-2-[2-(1,5-dimethyl-3-piperidyl)-1,3-benzothiazol-5-yl]-5-methyl-piperidine-1-carboxylate (0.21 g, 473.35 mol) in DCM and stirred at 25° C. for 6 hr. Reaction mixture was evaporated, triturate with water (2×50 mL), combined water phase filtered through cotton wool, pH adjusted to 11 with NaOH (20%, 10 mL), extracted with DCM (3×30 mL), dried over Na₂SO₄, concentrated to give 2-(1,5-dimethyl-3-piperidyl)-5-[(2R,5S)-5-methyl-2-piperidyl]-1,3-benzothiazole (0.16 g, 465.76 mol, 98.39% yield) (racemic mixture of diastereomers˜1:3).

LCMS(ESI): [M]⁺ m/z: calcd 343.2; found 344.2; Rt=1.646 min.

### Step 9: Synthesis of N-(6-amino-5-ethylpyridin-3-yl)-2-((2R,5S)-2-(2-(1,5-dimethylpiperidin-3-yl)benzo[d]thiazol-5-yl)-5-methylpiperidin-1-yl)-2-oxoacetamide (Compound 118)

Prepared by general procedure scheme 4.1 step 6B. Yield: 45 mg (18.07%).

HPLC conditions: Column: XBridge BEH C18 100*19 mm, 5 microM; 0-1-6 min 40-40-90% water-MeOH+0.1% NH₄OH, flow: 30 ml/min; (loading pump 4 ml/min MeOH).

Compound 118: ¹H NMR (600 MHz, DMSO-d₆) δ (ppm) 0.89 (d, 3H), 0.99-1.20 (m, 7H), 1.29-1.41 (m, 1H), 1.52 (t, 1H), 1.59-2.20 (m, 7H), 2.22 (s, 3H), 2.24-2.37 (m, 2H), 2.38-2.42 (m, 1H), 2.75-3.20 (m, 3H), 3.46-4.05 (m, 1H), 5.26-5.74 (m, 3H), 7.28-7.54 (m, 2H), 7.81-7.94 (m, 1H), 7.96-8.10 (m, 2H), 10.46-10.61 (m, 1H).

LCMS(ESI): [M]⁺ m/z: calcd 534.2; found 535.2; Rt=2.404 min.

### Step 10: Chiral Separation (Compound 75 and Compound 23)

Racemic N-(6-amino-5-ethyl-3-pyridyl)-2-oxo-2-[(2R,5S)-5-methyl-2-[2-[1,5-dimethyl-3-piperidyl]-1,3-benzothiazol-5-yl]-1-piperidyl]acetamide (0.045 g, 84.16 mol) was chiral separated (Column: Chiralpak IC (250*20 mm, 5 mkm), mobile phase: IPA-MeOH, 50-50, flow rate: 12 ml/min) to obtain N-(6-amino-5-ethyl-3-pyridyl)-2-oxo-2-[(2R,5S)-5-methyl-2-[2-[(3S,5S)-1,5-dimethyl-3-piperidyl]-1,3-benzothiazol-5-yl]-1-piperidyl]acetamide (12.8 mg, 23.94 mol, 28.44% yield) (RT=30.305 min) and N-(6-amino-5-ethyl-3-pyridyl)-2-oxo-2-[(2R,5S)-5-methyl-2-[2-[(3R,5R)-1,5-dimethyl-3-piperidyl]-1,3-benzothiazol-5-yl]cyclohexyl]acetamide (12.1 mg, 22.67 mol, 26.94% yield) (RT=37.67 min).

Rel Time for Compound 75 in analytical conditions (column: IC, MeOH-IPA, 50-50, 0.6 ml/min as mobile phase) 30.30 min and for Compound 23 37.65 min.

Compound 75: Retention time: 30.30 min

¹H NMR (600 MHz, DMSO-d₆) δ (ppm) 0.90 (m, 3H), 1.03 (m, 3H), 1.13 (m, 3H), 1.36 (m, 1H), 1.52 (m, 1H), 1.70 (m, 1H), 1.79 (m, 1H), 1.87 (m, 1H), 2.00 (m, 1H), 2.11 (m, 2H), 2.22 (s, 4H), 2.36 (m, 4H), 2.78 (m, 2H), 3.16 (m, 1H), 3.85 (m, 1H), 5.65 (m, 3H), 7.41 (m, 2H), 7.89 (m, 1H), 8.04 (m, 2H), 10.56 (m, 1H).

LCMS(ESI): [M]⁺ m/z: calcd 534.2; found 535.2; Rt=2.338 min.

Compound 23: Retention time: 37.65 min

¹H NMR (600 MHz, DMSO-d₆) δ (ppm) 0.89 (d, 3H), 1.02 (m, 3H), 1.13 (m, 3H), 1.36 (m, 1H), 1.52 (m, 1H), 1.70 (m, 1H), 1.84 (m, 2H), 1.99 (m, 1H), 2.11 (m, 2H), 2.22 (s, 4H), 2.37 (m, 4H), 2.78 (m, 2H), 3.16 (m, 1H), 3.66 (m, 1H), 5.65 (m, 3H), 7.41 (m, 2H), 7.89 (m, 1H), 8.04 (m, 2H), 10.56 (m, 1H).

LCMS(ESI): [M]⁺ m/z: calcd 534.2; found 535.2; Rt=2.330 min.

### Example 29. Compound 74 and Compound 36 2-methoxy-5-(2-((2R,5S)-5-methyl-2-(2-(1,4,4-trimethylpyrrolidin-3-yl)benzo[d]thiazol-5-yl)piperidin-1-yl)-2-oxoacetamido)nicotinamide

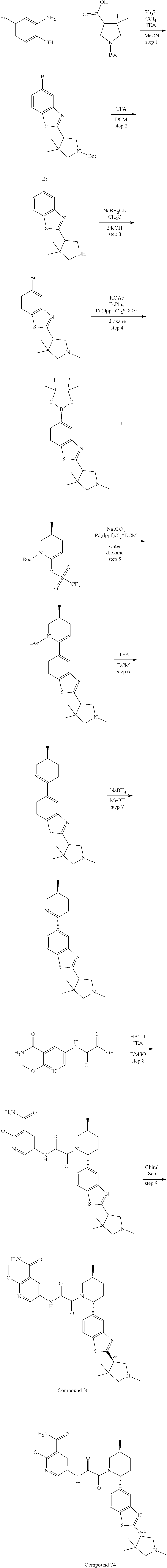

### Step 1: Synthesis of tert-butyl 4-(5-bromobenzo[d]thiazol-2-yl)-3,3-dimethylpyrrolidine-1-carboxylate

Triphenylphosphine (7.76 g, 29.59 mmol) was added in one portion to the solution of 1-tert-butoxycarbonyl-4,4-dimethyl-pyrrolidine-3-carboxylic acid (3 g, 12.33 mmol), 2-amino-4-bromo-benzenethiol (2.52 g, 12.33 mmol), carbon tetrachloride (11.00 g, 71.52 mmol, 6.92 mL) and TEA (6.24 g, 61.65 mmol, 8.59 mL). Resulting reaction mixture was briefly warmed up to approximately 50-60° C. due to exothermic reaction. After that, it was stirred at 30° C. for 12 hr. Then, volatiles were removed under reduced pressure and residue was triturated with MTBE (100 ml). Resulting precipitate was filtered off. Filtrate was concentrated under reduced pressure and residue was purified by gradient column chromatography (SiO₂, gradient Hexane-MTBE), to affording tert-butyl 4-(5-bromo-1,3-benzothiazol-2-yl)-3,3-dimethyl-pyrrolidine-1-carboxylate (1.5 g, 3.65 mmol, 29.57% yield).

LCMS(ESI): [M-t-Bu]⁺ m/z: calcd 355.2; found 356.2; Rt=1.754 min.

### Step 2: Synthesis of 5-bromo-2-(4,4-dimethylpyrrolidin-3-yl)benzo[d]thiazole

tert-Butyl 4-(5-bromo-1,3-benzothiazol-2-yl)-3,3-dimethyl-pyrrolidine-1-carboxylate (1.5 g, 3.65 mmol) was dissolved in mixture of DCM (15 mL) and TFA (15 g, 131.55 mmol, 10.14 mL), then stirred for 1 hr. The reaction mixture was concentrated in vacuum, the residue was treated with aq. solution of NaHCO₃ and desired product was extracted with DCM (2*40 ml), dried over Na₂SO₄, evaporated in vacuum to give 5-bromo-2-(4,4-dimethylpyrrolidin-3-yl)-1,3-benzothiazole (1.1 g, 3.53 mmol, 96.92% yield).

LCMS(ESI): [M]⁺ m/z: calcd 311.2; found 312.2; Rt=1.016 min.

### Step 3: Synthesis of 5-bromo-2-(1,4,4-trimethylpyrrolidin-3-yl)benzo[d]thiazole

Sodium cyan borohydride (1.11 g, 17.67 mmol) was added to a mixture of 5-bromo-2-(4,4-dimethylpyrrolidin-3-yl)-1,3-benzothiazole (1.1 g, 3.53 mmol) and formalin (1.43 g, 17.67 mmol, 1.32 mL, 37% purity) in MeOH (20 mL) and stirred overnight, then concentrated. The residue was treated with aq. solution of NaHCO₃ and desired product was extracted with 2*30 ml of DCM, dried over Na₂SO₄ and concentrated in vacuum to give 5-bromo-2-(1,4,4-trimethylpyrrolidin-3-yl)-1,3-benzothiazole (0.8 g, crude).

LCMS(ESI): [M]⁺ m/z: calcd 325.2; found 326.2; Rt=1.091 min.

### Step 4: Synthesis of 5-(4,4,5,5-tetramethyl-1,3,2-dioxaborolan-2-yl)-2-(1,4,4-trimethylpyrrolidin-3-yl)benzo[d]thiazole

Prepared by general procedure scheme 4.1 step 2. Yield: 1.25 g of crude.

LCMS(ESI): [M]⁺ m/z: calcd 372.2; found 373.2; Rt=1.212 min.

### Step 5: Synthesis of (3S)-tert-butyl 3-methyl-6-(2-(1,4,4-trimethylpyrrolidin-3-yl)benzo[d]thiazol-5-yl)-3,4-dihydropyridine-1(2H)-carboxylate

Prepared by general procedure scheme 4.1 step 3. Yield: 1.64 g of crude.

LCMS(ESI): [M]⁺ m/z: calcd 441.2; found 442.2; Rt=1.341 min.

### Step 6: Synthesis of 5-((S)-5-methyl-3,4,5,6-tetrahydropyridin-2-yl)-2-(1,4,4-trimethylpyrrolidin-3-yl)benzo[d]thiazole

Prepared by general procedure scheme 4.1 step 4. Yield: 1.15 g of crude.

LCMS(ESI): [M]⁺ m/z: calcd 341.2; found 342.2; Rt=0.710 min.

### Step 7: Synthesis of 5-((2R,5S)-5-methylpiperidin-2-yl)-2-(1,4,4-trimethylpyrrolidin-3-yl)benzo[d]thiazole

Prepared by general procedure scheme 4.1 step 5. Yield: 0.9 g of crude.

LCMS(ESI): [M]⁺ m/z: calcd 343.2; found 344.2; Rt=0.758 min.

### Step 8: Synthesis of 2-methoxy-5-(2-((2R,5S)-5-methyl-2-(2-(1,4,4-trimethylpyrrolidin-3-yl)benzo[d]thiazol-5-yl)piperidin-1-yl)-2-oxoacetamido)nicotinamide

Prepared by general procedure scheme 4.1 step 6A. Yield: 78.2 mg (21.14%).

HPLC conditions: Column: SunFire C18 100*19 mm, 5 microM; 2-10 min 30% water-MeCN+FA, flow: 30 ml/min; (loading pump 4 ml/min MeCN).

LCMS(ESI): [M]⁺ m/z: calcd 564.2; found 565.2; Rt=2.708 min.

### Step 9: Chiral Separation (Compound 74 and Compound 36)

Racemic 2-methoxy-5-(2-((2R,5S)-5-methyl-2-(2-(1,4,4-trimethylpyrrolidin-3-yl)benzo[d]thiazol-5-yl)piperidin-1-yl)-2-oxoacetamido)nicotinamide (65 mg, 115.11 umol) was chiral separated (Column: Chiralpak IA III (250*20, 5 mkm); IPA-MeOH, 50-50; 12 ml/min) to obtain 2-methoxy-5-(2-((2R,5S)-5-methyl-2-(2-((S)-1,4,4-trimethylpyrrolidin-3-yl)benzo[d]thiazol-5-yl)piperidin-1-yl)-2-oxoacetamido) nicotinamide (18.1 mg, 32.50 μmol) and 2-methoxy-5-(2-((2R,5S)-5-methyl-2-(2-((R)-1,4,4-trimethylpyrrolidin-3-yl)benzo[d]thiazol-5-yl)piperidin-1-yl)-2-oxoacetamido) nicotinamide (22.8 mg, 40.38 μmol).

Rel Time for Compound 74 in analytical conditions (column: IA, IPA-MeOH, 50-50, 0.6 ml/min as mobile phase) 27.43 min and for Compound 36 44.45 min.

### Compound 74: Retention Time: 27.43 Min

¹H NMR (600 MHz, DMSO-d₆) δ (ppm) 0.68-0.77 (m, 3H), 1.01-1.09 (m, 3H), 1.22-1.27 (m, 3H), 1.32-1.42 (m, 1H), 1.67-1.78 (m, 1H), 1.83-1.98 (m, 1H), 2.08-2.22 (m, 1H), 2.29-2.36 (m, 3H), 2.83-2.86 (m, 0.3H), 2.87-3.23 (m, 3H), 3.24-3.27 (m, 0.7H), 3.33-3.38 (m, 2H), 3.43-3.54 (m, 1.7H), 3.89-4.00 (m, 3H), 4.02-4.06 (m, 0.3H), 5.24-5.72 (m, 1H), 7.34-7.44 (m, 1H), 7.64-7.78 (m, 2H), 7.86-7.94 (m, 1H), 8.00-8.08 (m, 1H), 8.37-8.61 (m, 2H), 10.95-11.21 (m, 1H).

LCMS(ESI): [M]⁺ m/z: calcd 564.2; found 565.2; Rt=1.648 min.

### Compound 36: Retention Time: 44.45 Min

¹H NMR (600 MHz, DMSO-d₆) δ (ppm) 0.70-0.79 (m, 3H), 0.97-1.08 (m, 3H), 1.21-1.28 (m, 3H), 1.32-1.42 (m, 1H), 1.67-1.79 (m, 1H), 1.85-1.94 (m, 1H), 2.03-2.31 (m, 2H), 2.31-2.33 (m, 3H), 2.38-2.46 (m, 2H), 3.00-3.08 (m, 2H), 3.21-3.27 (m, 1H), 3.46-3.54 (m, 1.7H), 3.90-3.99 (m, 3H), 4.02-4.06 (m, 0.3H), 5.22-5.75 (m, 1H), 7.33-7.44 (m, 1H), 7.66-7.78 (m, 2H), 7.85-7.95 (m, 1H), 7.99-8.08 (m, 1H), 8.36-8.60 (m, 2H), 11.01-11.27 (m, 1H).

LCMS(ESI): [M]⁺ m/z: calcd 564.2; found 565.2; Rt=1.643 min.

### Example 30. Compound 89 N-(6-amino-5-ethylpyridin-3-yl)-2-((2R,5S)-2-(2-(2-(dimethylamino)-2-methylpropyl)benzo[d]thiazol-5-yl)-5-methylpiperidin-1-yl)-2-oxoacetamide

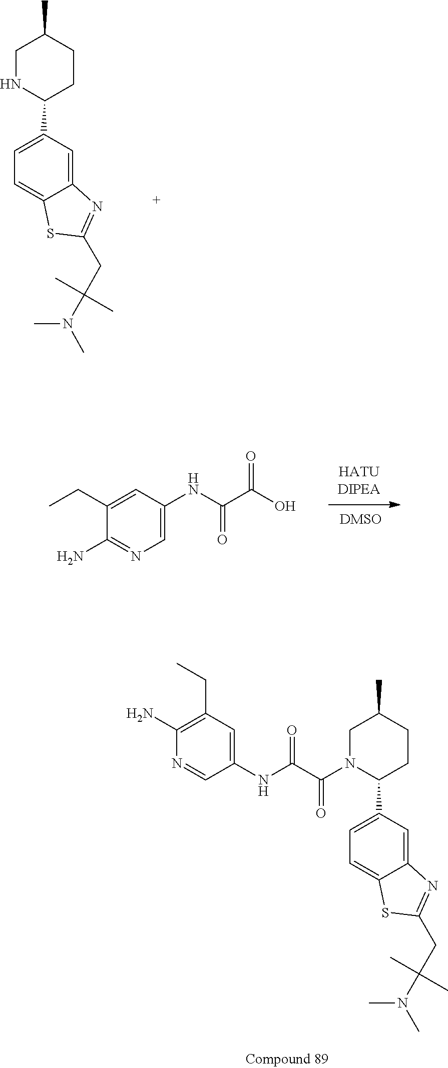

Synthesis of N,N,2-trimethyl-1-(5-((2R,5S)-5-methylpiperidin-2-yl)benzo[d]thiazol-2-yl)propan-2-amine is given by 3HHH.

Prepared by general procedure scheme 4.1 step 6A. Yield: 30 mg (6.34%).

HPLC conditions: Column: SunFire C18 100*19 mm, 5 microM; 2-10 min 35-50% water-MeOH+NH₃; (loading pump 4 ml/min MeOH+NH₃).

Compound 89: ¹H NMR (600 MHz, DMSO-d₆) δ (ppm) 1.02-1.14 (m, 12H), 1.30-1.41 (m, 1H), 1.66-1.76 (m, 1H), 1.82-1.93 (m, 1H), 2.03-2.17 (m, 1H), 2.26 (s, 6H), 2.28-2.34 (m, 1H), 2.36-2.44 (m, 2H), 2.76-2.81 (m, 0.3H), 3.17-3.23 (m, 2H), 3.26-3.28 (m, 0.7H), 3.46-4.09 (m, 1H), 5.24-5.61 (m, 1H), 5.61-5.73 (m, 2H), 7.27-7.40 (m, 1H), 7.43-7.55 (m, 1H), 7.81-7.89 (m, 1H), 7.97-8.10 (m, 2H), 10.55 (m, 1H).

LCMS(ESI): [M]⁺ m/z: calcd 522.2; found 523.2; Rt=2.235 min.

### Example 31. Compound 18 N-(6-amino-5-ethylpyridin-3-yl)-2-((2R,5S)-2-(2-((1,3-dimethylpiperidin-4-yl)methyl)benzo[d]thiazol-5-yl)-5-methylpiperidin-1-yl)-2-oxoacetamide

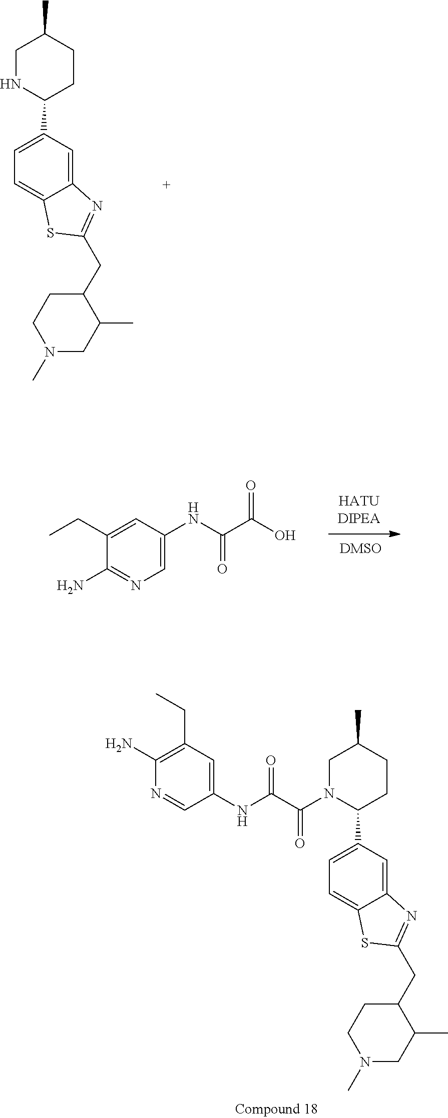

Synthesis of 2-((1,3-dimethylpiperidin-4-yl)methyl)-5-((2R,5S)-5-methylpiperidin-2-yl)benzo[d]thiazole is given by 3III.

Prepared by general procedure scheme 4.1 step 6B. Yield: 102 mg (39.10%).

HPLC conditions: Column: SunFire C18 100*19 mm, 5 microM; 2-10 min 30-65 MeOH+NH₃; (loading pump 4 ml/min MeOH).

Compound 18: ¹H NMR (600 MHz, DMSO-d₆) δ (ppm) 0.96 (m, 3H), 1.03 (m, 3H), 1.10 (m, 3H), 1.31 (m, 1H), 1.47 (m, 3H), 1.72 (m, 2H), 1.83 (m, 2H), 2.01 (s, 1H), 2.08 (m, 4H), 2.18 (m, 1H), 2.36 (m, 4H), 2.68 (m, 1H), 2.82 (m, 1H), 3.00 (m, 1H), 3.69 (m, 1H), 5.64 (m, 3H), 7.42 (m, 2H), 7.87 (m, 1H), 8.03 (m, 2H), 10.55 (m, 1H).

LCMS(ESI): [M]⁺ m/z: calcd 548.2; found 549.2; Rt=1.848 min.

### Example 32. Compound 45 and Compound 105 N-(6-amino-5-ethylpyridin-3-yl)-2-((2R,5S)-5-methyl-2-(2-(1,5,5-trimethylpiperidin-3-yl)benzo[d]thiazol-5-yl)piperidin-1-yl)-2-oxoacetamide

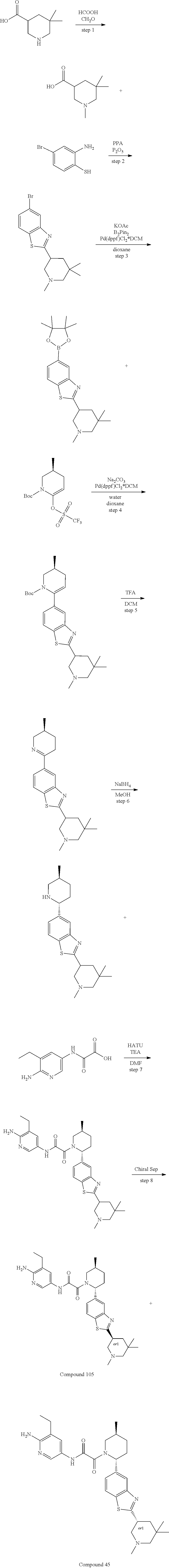

### Step 1: Synthesis of 1,5,5-trimethylpiperidine-3-carboxylic acid

Formaldehyde, 37% w/w aq. soln., stab. with 7-8% MeOH (2.10 g, 25.82 mmol, 1.93 mL, 37% purity) was added to the solution of 5,5-dimethylpiperidine-3-carboxylic acid (2.5 g, 12.91 mmol, HCl) in formic acid (85% in water) (18.30 g, 337.97 mmol, 15 mL, 85% purity). Catalytic amount of Na₂CO₃ (20-30 mg) was added and resulting solution was stirred at 100° C. for 24 hr. Then, it was concentrated under reduced pressure. Residue was re-dissolved and co-evaporated with conc. hydrochloric acid (10 ml) in order to remove traces of paraform and formic acid. Resulting white solid was dried in vacuum, affording 1,5,5-trimethylpiperidine-3-carboxylic acid (2.5 g, 12.04 mmol, 93.25% yield, HCl).

LCMS(ESI): [M]⁺ m/z: calcd 171.2; found 172.2; Rt=0.374 min.

### Step 2: Synthesis of 5-bromo-2-(1,5,5-trimethylpiperidin-3-yl)benzo[d]thiazole

Prepared by general procedure scheme 4.1 step 1A. Yield: 3.71 g of crude.

LCMS(ESI): [M]⁺ m/z: calcd 339.2; found 340.2; Rt=0.786 min.

### Step 3: Synthesis of 5-(4,4,5,5-tetramethyl-1,3,2-dioxaborolan-2-yl)-2-(1,5,5-trimethylpiperidin-3-yl)benzo[d]thiazole

Prepared by general procedure scheme 4.1 step 2. Yield: 4.45 g of crude.

LCMS(ESI): [M]⁺ m/z: calcd 386.2; found 387.2; Rt=0.995 min.

### Step 4: Synthesis of (3S)-tert-butyl 3-methyl-6-(2-(1,5,5-trimethylpiperidin-3-yl)benzo[d]thiazol-5-yl)-3,4-dihydropyridine-1(2H)-carboxylate

Prepared by general procedure scheme 4.1 step 3. Yield: 6.5 g of crude.

LCMS(ESI): [M]⁺ m/z: calcd 455.2; found 456.2; Rt=1.335 min.

### Step 5: Synthesis of 5-((S)-5-methyl-3,4,5,6-tetrahydropyridin-2-yl)-2-(1,5,5-trimethylpiperidin-3-yl)benzo[d]thiazole

Prepared by general procedure scheme 4.1 step 4. Yield: 3.5 g of crude.

LCMS(ESI): [M]⁺ m/z: calcd 355.2; found 356.2; Rt=0.774 min.

### Step 6: Synthesis of 5-((2R,5S)-5-methylpiperidin-2-yl)-2-(1,5,5-trimethylpiperidin-3-yl)benzo[d]thiazole

Prepared by general procedure scheme 4.1 step 5. Yield: 3.1 mg of crude.

LCMS(ESI): [M]⁺ m/z: calcd 357.2; found 358.2; Rt=0.743 min.

### Step 7: Synthesis of N-(6-amino-5-ethylpyridin-3-yl)-2-((2R,5S)-5-methyl-2-(2-(1,5,5-trimethylpiperidin-3-yl)benzo[d]thiazol-5-yl)piperidin-1-yl)-2-oxoacetamide

Prepared by general procedure scheme 4.1 step 6A. Yield: 498 mg (60.09%).

HPLC conditions: Column: XBridge C18 100*19 mm, 5 microM; 0-5 min 25-75% water-MeCN+0.1% NH₄OH, flow: 30 ml/min; (loading pump 4 ml/min MeCN).

¹H NMR (600 MHz, DMSO-d₆) δ (ppm)

LCMS(ESI): [M]⁺ m/z: calcd 548.2; found 549.2; Rt=2.508 min.

### Step 8: Chiral Separation (Compound 45 and Compound 105)

Racemic N-(6-amino-5-ethyl-3-pyridyl)-2-oxo-2-[(2R,5S)-5-methyl-2-[2-(1,5,5-trimethyl-3-piperidyl)-1,3-benzothiazol-5-yl]-1-piperidyl]acetamide (360 mg, 656.05 μmol) was chiral separated (Column: Chiralpak IC III (250*20 mm, 5 mkm); Mobile phase: IPA-MeOH, 50-50. Flow Rate: 12 mL/min) to obtain N-(6-amino-5-ethyl-3-pyridyl)-2-oxo-2-[(2R,5S)-5-methyl-2-[2-[(3S)-1,5,5-trimethyl-3-piperidyl]-1,3-benzothiazol-5-yl]-1-piperidyl]acetamide (134 mg, 244.20 μmol, 74.44% yield) (RT=24.67 min) and N-(6-amino-5-ethyl-3-pyridyl)-2-oxo-2-[(2R,5S)-5-methyl-2-[2-[(3R)-1,5,5-trimethyl-3-piperidyl]-1,3-benzothiazol-5-yl]-1-piperidyl]acetamide (123 mg, 224.15 μmol, 68.33% yield) (RT=29.99 min).

Rel Time for Compound 45 in analytical conditions (column: IC, MeOH-IPA, 50-50, 0.6 ml/min as mobile phase) 28.11 min and for Compound 105 23.17 min.

### Compound 45: Retention Time: 28.11 Min

¹H NMR (600 MHz, DMSO-d₆) δ (ppm) 0.97 (m, 6H), 1.10 (m, 6H), 1.37 (m, 2H), 1.67 (m, 2H), 1.90 (m, 3H), 2.07 (m, 1H), 2.19 (s, 3H), 2.39 (m, 4H), 2.97 (m, 2H), 3.66 (m, 2H), 5.65 (m, 3H), 7.41 (m, 2H), 7.88 (m, 1H), 8.04 (m, 2H), 10.56 (m, 1H).

LCMS(ESI): [M]⁺ m/z: calcd 548.2; found 549.2; Rt=2.170 min.

Compound 105: Retention time: 23.17 min

¹H NMR (600 MHz, DMSO-d₆) δ (ppm) 0.97 (m, 6H), 1.10 (m, 6H), 1.36 (m, 2H), 1.67 (m, 2H), 1.90 (m, 3H), 2.07 (m, 1H), 2.19 (s, 3H), 2.39 (m, 4H), 2.96 (m, 2H), 3.66 (m, 2H), 5.64 (m, 3H), 7.41 (m, 2H), 7.88 (m, 1H), 8.04 (m, 2H), 10.56 (m, 1H).

LCMS(ESI): [M]⁺ m/z: calcd 548.2; found 549.2; Rt=2.147 min.

### Example 33. Compound 61 5-(2-((2R,5S)-2-(2-(1,5-dimethyl-1,2,3,6-tetrahydropyridin-4-yl)benzo[d]thiazol-5-yl)-5-methylpiperidin-1-yl)-2-oxoacetamido)-2-methoxynicotinamide

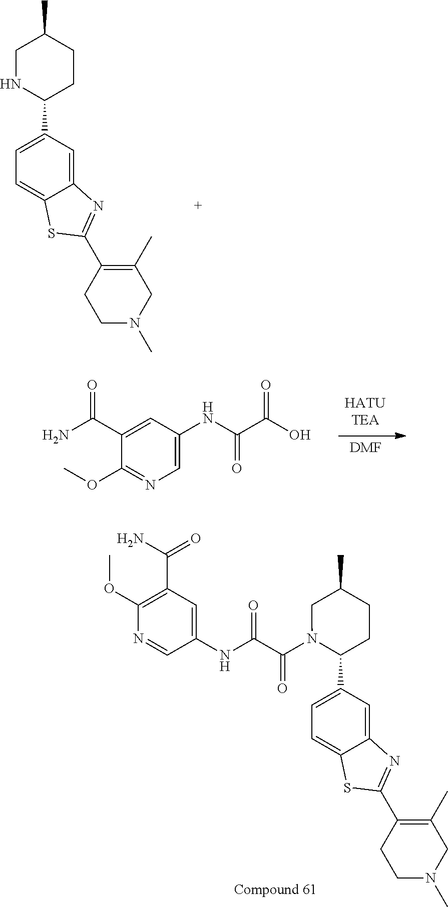

The synthesis of 2-(1,5-dimethyl-1,2,3,6-tetrahydropyridin-4-yl)-5-((2R,5S)-5-methylpiperidin-2-yl)benzo[d]thiazole is given by 3JJJ.

Prepared by general procedure scheme 4.1 step 6A. Yield: 4.4 mg (1.78%).

HPLC conditions: Column: XBridge BEH C18 100*19 mm, 5 microM; 0-6 min 50-70% water-MeOH+0.1% NH₄OH; (loading pump 4 ml/min MeOH).

Compound 61: ¹H NMR (600 MHz, DMSO-d₆) δ (ppm) 1.02-1.08 (m, 3H), 1.32-1.42 (m, 1H), 1.69-1.78 (m, 1H), 1.86-1.95 (m, 1H), 2.04-2.16 (m, 4H), 2.18-2.33 (m, 4H), 2.54-2.57 (m, 2H), 2.62-2.66 (m, 2H), 2.83-3.04 (m, 3H), 3.52-4.06 (m, 4H), 5.27-5.75 (m, 1H), 7.36-7.47 (m, 1H), 7.66-7.76 (m, 2H), 7.88-7.95 (m, 1H), 8.04-8.12 (m, 1H), 8.39-8.60 (m, 2H), 10.67 (s, 1H).

LCMS(ESI): [M]⁺ m/z: calcd 562.2; found 563.2; Rt=2.688 min.

### Example 34. Compound 77, Compound 30 and Compound 65 of 2-methoxy-5-(2-((2R,5S)-5-methyl-2-(2-(1,5,5-trimethylpyrrolidin-3-yl)benzo[d]thiazol-5-yl)piperidin-1-yl)-2-oxoacetamido)nicotinamide

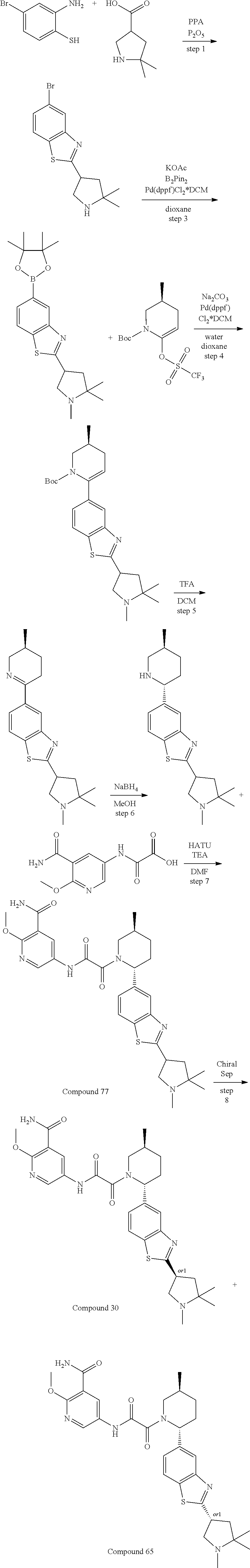

### Step 1: Synthesis of 5-bromo-2-(5,5-dimethylpyrrolidin-3-yl)benzo[d]thiazole

Prepared by general procedure scheme 4.1 step 1A. Yield: 9.5 g (98.88%).

LCMS(ESI): [M]⁺ m/z: calcd 311.2; found 312.2; Rt=0.949 min.

### Step 2: Synthesis of 5-bromo-2-(1,5,5-trimethylpyrrolidin-3-yl)benzo[d]thiazole

Formaldehyde, 37% w/w aq. soln., stab. with 7-8% MeOH (3.47 g, 42.73 mmol, 3.20 mL, 37% purity) and acetic acid (3.67 g, 61.05 mmol, 3.49 mL) were added to a stirred solution of 5-bromo-2-(5,5-dimethylpyrrolidin-3-yl)-1,3-benzothiazole (9.5 g, 30.52 mmol) in MeOH (250 mL) at 25° C. The resulting mixture was stirred at 25° C. for 1 hr, then sodium cyan borohydride (1.92 g, 30.52 mmol) was added in one portion at 25° C. (foaming!). The reaction mixture was stirred at 25° C. for 18 hr, and then concentrated in vacuum. The residue was diluted with 10% aqueous sodium hydroxide solution (100 ml) and extracted with DCM (2*100 ml). The combined organic extracts were dried over sodium sulphate and concentrated in vacuum to afford crude 5-bromo-2-(1,5,5-trimethylpyrrolidin-3-yl)-1,3-benzothiazole (9.5 g, 29.21 mmol, 95.69% yield) as light-yellow solid, which was used directly in the next step.

LCMS(ESI): [M]⁺ m/z: calcd 325.2; found 326.2; Rt=2.518 min.

### Step 3: Synthesis of 5-(4,4,5,5-tetramethyl-1,3,2-dioxaborolan-2-yl)-2-(1,5,5-trimethylpyrrolidin-3-yl)benzo[d]thiazole

Prepared by general procedure scheme 4.1 step 2. Yield: 5.7 g of crude.

LCMS(ESI): [M]⁺ m/z: calcd 372.2; found 373.2; Rt=1.171 min.

### Step 4: Synthesis of (3S)-tert-butyl 3-methyl-6-(2-(1,5,5-trimethylpyrrolidin-3-yl)benzo[d]thiazol-5-yl)-3,4-dihydropyridine-1(2H)-carboxylate

Prepared by general procedure scheme 4.1 step 3. Yield: 16.5 g of crude.

LCMS(ESI): [M]⁺ m/z: calcd 441.2; found 442.2; Rt=1.289 min.

### Step 5: Synthesis of 5-((S)-5-methyl-3,4,5,6-tetrahydropyridin-2-yl)-2-(1,5,5-trimethylpyrrolidin-3-yl)benzo[d]thiazole

Prepared by general procedure scheme 4.1 step 4. Yield: 4.7 g of crude.

LCMS(ESI): [M]⁺ m/z: calcd 341.2; found 342.2; Rt=0.635 min.

### Step 6: Synthesis of 5-((2R,5S)-5-methylpiperidin-2-yl)-2-(1,5,5-trimethylpyrrolidin-3-yl)benzo[d]thiazole

Sodium borohydride (780.94 mg, 20.64 mmol, 727.13 μL) was added in one portion at 0° C. to a stirred solution of 5-[(3S)-3-methyl-2,3,4,5-tetrahydropyridin-6-yl]-2-(1,5,5-trimethylpyrrolidin-3-yl)-1,3-benzothiazole (4.7 g, 13.76 mmol) in MeOH (60 mL). The reaction mixture was stirred at 0° C. for 1 hr, and then concentrated in vacuum. The residue was diluted with water (25 ml) and extracted with DCM (2*50 ml). The combined organic extracts were dried over sodium sulphate and concentrated in vacuum to afford crude 5-[(2R,5S)-5-methyl-2-piperidyl]-2-(1,5,5-trimethylpyrrolidin-3-yl)-1,3-benzothiazole (4 g, 11.64 mmol, 84.61% yield) as light-brown gum, which was used directly in the next step. Additionally, 1 g of crude amine was purified by reverse phase HPLC (column: XBridge C18 100×19 mm, 5 um; mobile phase: 35-60% 0-5 min H₂O/MeCN/0.1% NH₄OH, flow rate: 30 ml/min (loading pump 4 ml/min MeCN)) to afford pure 5-[(2R,5S)-5-methyl-2-piperidyl]-2-(1,5,5-trimethylpyrrolidin-3-yl)-1,3-benzothiazole (530 mg, 1.54 mmol, 11.21% yield) as red gum.

LCMS(ESI): [M]⁺ m/z: calcd 343.2; found 344.2; Rt=0.789 min.

### Step 7: Synthesis of 2-methoxy-5-(2-((2R,5S)-5-methyl-2-(2-(1,5,5-trimethylpyrrolidin-3-yl)benzo[d]thiazol-5-yl)piperidin-1-yl)-2-oxoacetamido)nicotinamide

Prepared by general procedure scheme 4.1 step 6A. Yield: 184 mg (44.77%).

HPLC conditions: Column: YMC Triart C18 100*20 mm, 5 microM; 0-1-6 min 60-60-85% water-MeOH+0.1% NH₄OH, flow: 30 ml/min; (loading pump 4 ml/min MeOH).

Compound 77: ¹H NMR (600 MHz, DMSO-d₆) δ (ppm) 0.96-1.01 (m, 3H), 1.01-1.06 (m, 3H), 1.06-1.11 (m, 3H), 1.29-1.42 (m, 1H), 1.65-1.79 (m, 1H), 1.83-1.95 (m, 1H), 1.95-2.04 (m, 1H), 2.06-2.15 (m, 1H), 2.17 (s, 3H), 2.20-2.33 (m, 2H), 2.81-2.87 (m, 0.3H), 3.00-3.13 (m, 2H), 3.23 (s, 0.7H), 3.48-3.55 (m, 0.7H), 3.74-3.84 (m, 1H), 3.90-4.01 (m, 3H), 4.01-4.07 (m, 0.3H), 5.23-5.76 (m, 1H), 7.31-7.42 (m, 1H), 7.66-7.80 (m, 2H), 7.82-7.91 (m, 1H), 7.97-8.09 (m, 1H), 8.38-8.65 (m, 2H), 10.86-11.29 (m, 1H).

LCMS(ESI): [M]⁺ m/z: calcd 564.2; found 565.2; Rt=2.782 min.

### Step 8: Chiral Separation (Compound 30 and Compound 65)

Racemic 2-methoxy-5-[[2-oxo-2-[(2R,5S)-5-methyl-2-[2-(1,5,5-trimethylpyrrolidin-3-yl)-1,3-benzothiazol-5-yl]-1-piperidyl]acetyl]amino]pyridine-3-carboxamide (90 mg, 159.38 μmol) was chiral separated (Column: Chiralpak IC III (250*20 mm, 5 mkm); Mobile phase: Hexane-IPA-MeOH, 50-25-25; Flow rate: 12 ml/min) to obtain 2-methoxy-5-[[2-oxo-2-[(2R,5S)-5-methyl-2-[2-[(3R)-1,5,5-trimethylpyrrolidin-3-yl]-1,3-benzothiazol-5-yl]-1-piperidyl]acetyl]amino]pyridine-3-carboxamide (28 mg, 49.58 mol, 31.11% yield) (RT=90.59 min) and 2-methoxy-5-[[2-oxo-2-[(2R,5S)-5-methyl-2-[2-[(35)-1,5,5-trimethylpyrrolidin-3-yl]-1,3-benzothiazol-5-yl]-1-piperidyl]acetyl]amino]pyridine-3-carboxamide (32 mg, 56.67 mol, 35.56% yield) (RT=80.06 min).

Rel Time for Compound 65 in analytical conditions (column: IC, IPA-MeOH, 50-50, 0.6 ml/min as mobile phase) 43.67 min and for Compound 30 38.43 min.

### Compound 65: Retention Time: 43.67 Min

¹H NMR (600 MHz, DMSO-d₆) δ (ppm) 0.96-1.01 (m, 3H), 1.02-1.08 (m, 6H), 1.30-1.41 (m, 1H), 1.66-1.76 (m, 1H), 1.82-1.94 (m, 1H), 1.94-2.03 (m, 1H), 2.03-2.16 (m, 1H), 2.16-2.19 (m, 3H), 2.20-2.34 (m, 2H), 2.82-3.24 (m, 3H), 3.49-4.05 (m, 5H), 5.20-5.79 (m, 1H), 7.27-7.43 (m, 1H), 7.65-7.79 (m, 2H), 7.80-7.91 (m, 1H), 7.97-8.08 (m, 1H), 8.38-8.62 (m, 2H), 10.99-11.14 (m, 1H).

LCMS(ESI): [M]⁺ m/z: calcd 564.2; found 565.2; Rt=2.544 min.

### Compound 30: Retention Time: 38.43 Min

¹H NMR (600 MHz, DMSO-d₆) δ (ppm) 0.99 (s, 3H), 1.01-1.09 (m, 6H), 1.29-1.41 (m, 1H), 1.65-1.75 (m, 1H), 1.78-1.94 (m, 1H), 1.94-2.14 (m, 2H), 2.15-2.19 (m, 3H), 2.20-2.34 (m, 2H), 2.80-3.12 (m, 3H), 3.45-4.08 (m, 5H), 5.25-5.72 (m, 1H), 7.30-7.44 (m, 1H), 7.66-7.77 (m, 2H), 7.80-7.88 (m, 1H), 7.97-8.07 (m, 1H), 8.36-8.62 (m, 2H), 10.99-11.14 (m, 1H).

LCMS(ESI): [M]⁺ m/z: calcd 564.2; found 565.2; Rt=2.529 min.

### Example 35. Compound 122 5-(2-((2R,5S)-2-(2-(1,4-dimethylpiperidin-4-yl)benzo[d]thiazol-5-yl)-5-methylpiperidin-1-yl)-2-oxoacetamido)-2-methoxynicotinamide

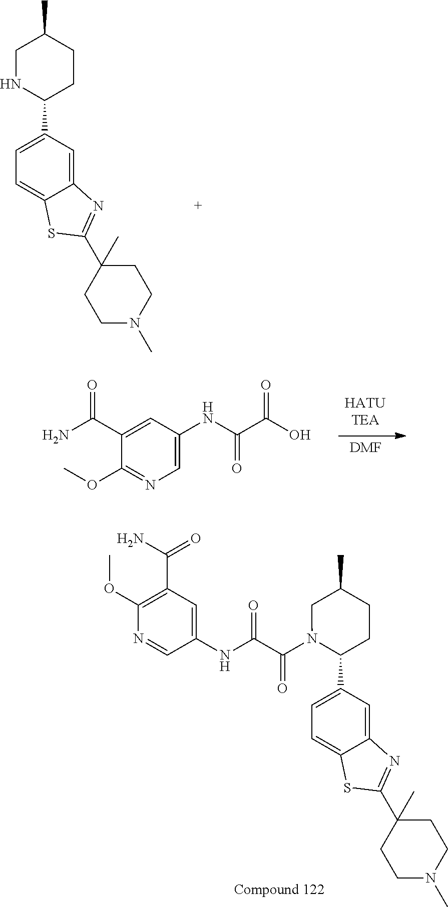

Synthesis of 2-(1,4-dimethylpiperidin-4-yl)-5-((2R,5S)-5-methylpiperidin-2-yl)benzo[d]thiazole is given by 3111.

Prepared by general procedure scheme 4.1 step 6A. Yield: 81 mg (24.64%).

HPLC conditions: Column: XBridge BEH C18 100*19 mm, 5 microM; 0-1-6 min 50-80% water-MeOH+0.1% NH₄OH; (loading pump 4 ml/min MeOH).

Compound 122: 1H NMR (DMSO-d6, 600 MHz): δ (ppm) 1.03-1.06 (m, 3H), 1.34-1.42 (m, 4H), 1.72-2.11 (m, 9H), 2.22-2.37 (m, 4H), 2.84-2.86 (m, 1H), 3.50-4.06 (m, 5H), 5.28-5.70 (m, 2H), 7.36-7.42 (m, 1H), 7.69-7.74 (m, 2H), 7.88-7.91 (m, 1H), 8.04-8.08 (m, 1H), 8.42-8.58 (m, 2H), 11.03-11.12 (m, 1H).

LCMS(ESI): [M]⁺ m/z: calcd 564.2; found 565.2; Rt=2.093 min.

### Example 36. Compound 81 5-(2-((2R,5S)-2-(2-(1-azabicyclo[2.2.1]heptan-4-yl)benzo[d]thiazol-5-yl)-5-methylpiperidin-1-yl)-2-oxoacetamido)-2-methoxynicotinamide

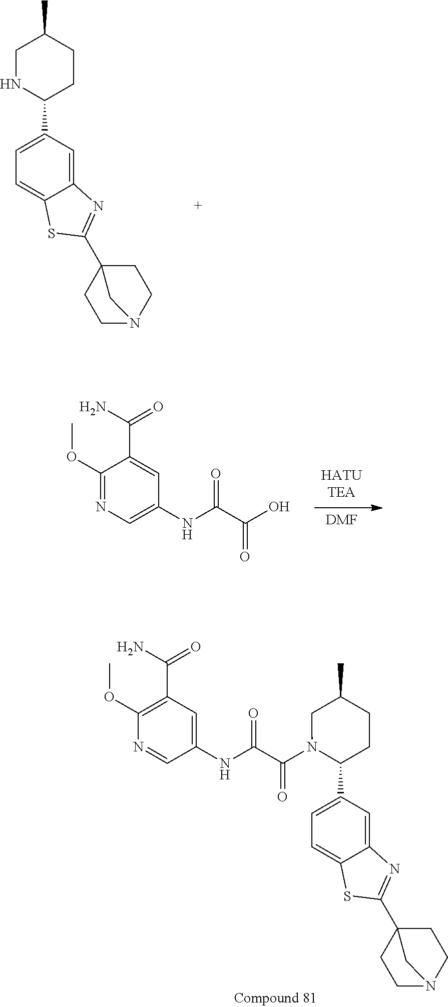

The synthesis of 2-(1-azabicyclo[2.2.1]heptan-4-yl)-5-((2R,5S)-5-methylpiperidin-2-yl)benzo[d]thiazole is given by 3KKK.

Prepared by general procedure scheme 4.1 step 6A. Yield: 61 mg (8.09%).

HPLC conditions: Column: YMC Triart C18 100*20 mm, 5 microM; 0-6 min 50-50% water-MeOH+0.1% NH₄OH, flow: 30 ml/min; (loading pump 4 ml/min MeOH).

Compound 81: ¹H NMR (600 MHz, DMSO-d₆) δ (ppm) 0.98-1.07 (m, 3H), 1.32-1.42 (m, 1H), 1.66-1.80 (m, 3H), 1.82-1.94 (m, 1H), 2.06-2.20 (m, 3H), 2.28-2.36 (m, 1H), 2.66-2.73 (m, 2H), 2.72-2.78 (m, 2H), 2.81-2.88 (m, 0.4H), 3.01-3.08 (m, 2H), 3.21-3.26 (m, 0.6H), 3.46-3.54 (m, 0.6H), 3.90-4.00 (m, 3H), 4.02-4.05 (m, 0.4H), 5.24-5.78 (m, 1H), 7.35-7.47 (m, 1H), 7.67-7.78 (m, 2H), 7.85-7.97 (m, 1H), 8.01-8.12 (m, 1H), 8.36-8.49 (m, 1H), 8.49-8.62 (m, 1H), 10.93-11.16 (m, 1H).

LCMS(ESI): [M]⁺ m/z: calcd 548.2; found 549.2; Rt=2.482 min.

### Example 37. Compound 90 and Compound 101 N-(6-amino-5-ethylpyridin-3-yl)-2-((2R,5S)-2-(2-(3-methoxy-1-methylpiperidin-4-yl)benzo[d]thiazol-5-yl)-5-methylpiperidin-1-yl)-2-oxoacetamide

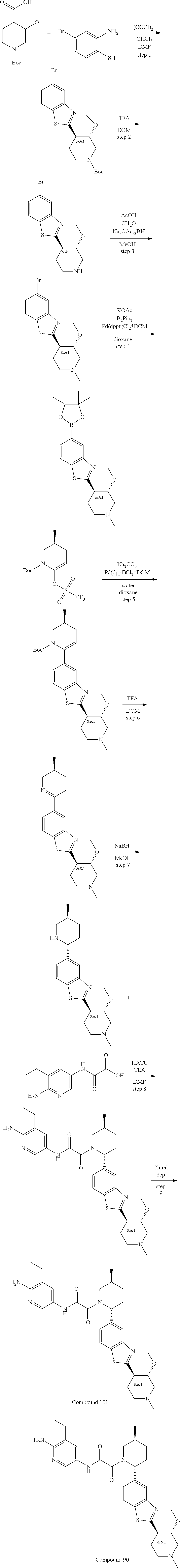

Step 1-7 are given by 3III.

### Step 8: Synthesis of N-(6-amino-5-ethylpyridin-3-yl)-2-((2R,5S)-2-(2-(3-methoxy-1-methylpiperidin-4-yl)benzo[d]thiazol-5-yl)-5-methylpiperidin-1-yl)-2-oxoacetamide

Prepared by general procedure scheme 4.1 step 6A. Yield: 78 mg (14.98%).

### HPLC Conditions:

1-st run: Column: XBridge BEH C18 100*19 mm, 5 microM; 0-1-5 min 40-40-60% water-MeOH+0.1% NH₄OH, flow: 30 ml/min; (loading pump 4 ml/min MeOH).

2-nd run: Column: XBridge BEH C18 100*19 mm, 5 microM; 0-1-6 min 10-10-50% water-MeCN+0.1% NH₄OH, flow: 30 ml/min; (loading pump 4 ml/min MeCN).

LCMS(ESI): [M]⁺ m/z: calcd 550.2; found 551.2; Rt=1.713 min.

### Step 9: Chiral Separation (Compound 90 and Compound 101)

Racemic N-(6-amino-5-ethyl-3-pyridyl)-2-oxo-2-[(2R,5S)-5-methyl-2-[2-[rac-(3S,4R)-3-methoxy-1-methyl-4-piperidyl]-1,3-benzothiazol-5-yl]-1-piperidyl]acetamide (78 mg, 141.63 mol) was chiral separated (Column: Chiralcel OJ-H (250*20 mm, 5 mkm); mobile phase: Hexane-IPA-MeOH, 50-25-25; flow rate: 12 ml/min) to afford crude 1st fraction (RT=11.784 min) and Compound 90 N-(6-amino-5-ethyl-3-pyridyl)-2-oxo-2-[(2R,5S)-5-methyl-2-[2-[(3R,4S)-3-methoxy-1-methyl-4-piperidyl]-1,3-benzothiazol-5-yl]-1-piperidyl]acetamide (24.5 mg, 44.49 mol, 31.41% yield) (second fraction, RT=29.027 min) as beige solid. The crude 1st fraction was re-purified by preparative chiral HPLC (column:Chiralpak IA-III (250*20, 5 mkm); mobile phase: IPA-MeOH, 50-50; flow rate: 10 ml/min) (RT=29.589 min) and then again re-purified by preparative chiral HPLC (column: Chiralcel OD-H (250*20, 5 mkm); mobile phase: Hexane-IPA-MeOH, 80-10-10; flow rate:14 ml/min) to afford Compound 101 N-(6-amino-5-ethyl-3-pyridyl)-2-oxo-2-[(2R,5S)-5-methyl-2-[2-[(3S,4R)-3-methoxy-1-methyl-4-piperidyl]-1,3-benzothiazol-5-yl]-1-piperidyl]acetamide (18 mg, 32.68 μmol, 23.08% yield) (RT=33.557 min) as yellow solid.

Rel Time for Compound 90 in analytical conditions (column: OJ-H, Hexane-MeOH-IPA, 50-25-25, 0.6 ml/min as mobile phase) 20.65 min and for Compound 101 12.95 min.

### Compound 90: Retention Time: 20.65 Min

¹H NMR (600 MHz, DMSO-d₆) δ (ppm) 1.01-1.06 (m, 3H), 1.06-1.17 (m, 3H), 1.30-1.41 (m, 1H), 1.67-1.75 (m, 1H), 1.84-1.94 (m, 2H), 2.04-2.17 (m, 3H), 2.17-2.24 (m, 4H), 2.30-2.35 (m, 1H), 2.38-2.45 (m, 2H), 2.65-2.86 (m, 2H), 2.86-3.16 (m, 2H), 3.21 (s, 3H), 3.47-3.52 (m, 0.7H), 3.69-3.78 (m, 1H), 4.01-4.11 (m, 0.3H), 5.17-5.62 (m, 1H), 5.62-5.77 (m, 2H), 7.31-7.43 (m, 1H), 7.43-7.57 (m, 1H), 7.82-7.93 (m, 1H), 7.98-8.15 (m, 2H), 10.45-10.70 (m, 1H).

LCMS(ESI): [M]⁺ m/z: calcd 550.2; found 551.2; Rt=1.139 min.

Compound 101: Retention time: 12.95 min

¹H NMR (600 MHz, DMSO-d₆) δ (ppm) 1.01-1.05 (m, 3H), 1.05-1.17 (m, 3H), 1.29-1.42 (m, 1H), 1.66-1.79 (m, 1H), 1.80-1.93 (m, 2H), 1.98-2.17 (m, 3H), 2.17-2.29 (m, 4H), 2.29-2.35 (m, 1H), 2.36-2.44 (m, 2H), 2.61-2.84 (m, 2H), 2.84-3.16 (m, 2H), 3.21 (s, 3H), 3.45-3.51 (m, 0.7H), 3.70-3.76 (m, 1H), 4.01-4.08 (m, 0.3H), 5.25-5.62 (m, 1H), 5.61-5.75 (m, 2H), 7.28-7.43 (m, 1H), 7.43-7.55 (m, 1H), 7.80-7.91 (m, 1H), 7.96-8.12 (m, 2H), 10.49-10.60 (m, 1H).

LCMS(ESI): [M]⁺ m/z: calcd 550.2; found 551.2; Rt=1.144 min.

### Example 38. Compound 64 5-(2-((2R,5S)-2-(2-((1,3-dimethylpiperidin-4-yl)methyl)benzo[d]thiazol-5-yl)-5-methylpiperidin-1-yl)-2-oxoacetamido)-2-methoxynicotinamide

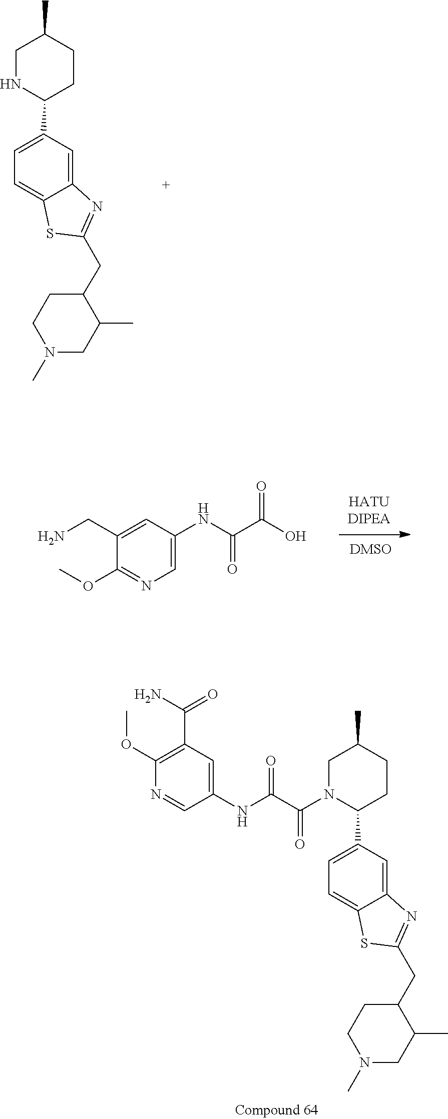

The synthesis of 2-((1,3-dimethylpiperidin-4-yl)methyl)-5-((2R,5S)-5-methylpiperidin-2-yl)benzo[d]thiazole is given by 3111.

Prepared by general procedure scheme 4.1 step 6B. Yield: 65 mg (23.62%).

HPLC conditions: Column: SunFire C18 100*19 mm, 5 microM; 2-10 min 30-65% water-MeOH+NH₃; (loading pump 4 ml/min MeOH).

Compound 64: ¹H NMR (600 MHz, DMSO-d₆) δ (ppm) 1.00 (m, 6H), 1.32 (m, 1H), 1.60 (m, 5H), 1.86 (m, 2H), 2.02 (s, 1H), 2.09 (m, 5H), 2.30 (m, 1H), 2.67 (m, 2H), 2.92 (m, 2H), 3.95 (m, 4H), 5.49 (d, 1H), 7.38 (m, 1H), 7.72 (m, 2H), 7.88 (m, 1H), 8.03 (m, 1H), 8.51 (m, 2H), 11.07 (m, 1H).

LCMS(ESI): [M]⁺ m/z: calcd 578.2; found 579.2; Rt=2.263 min.

### Example 39. Compound 50 and Compound 24 5-(2-((2R,5S)-2-(2-(3-(dimethylamino)cyclobutyl)benzo[d]thiazol-5-yl)-5-methylpiperidin-1-yl)-2-oxoacetamido)-2-methoxybenzamide

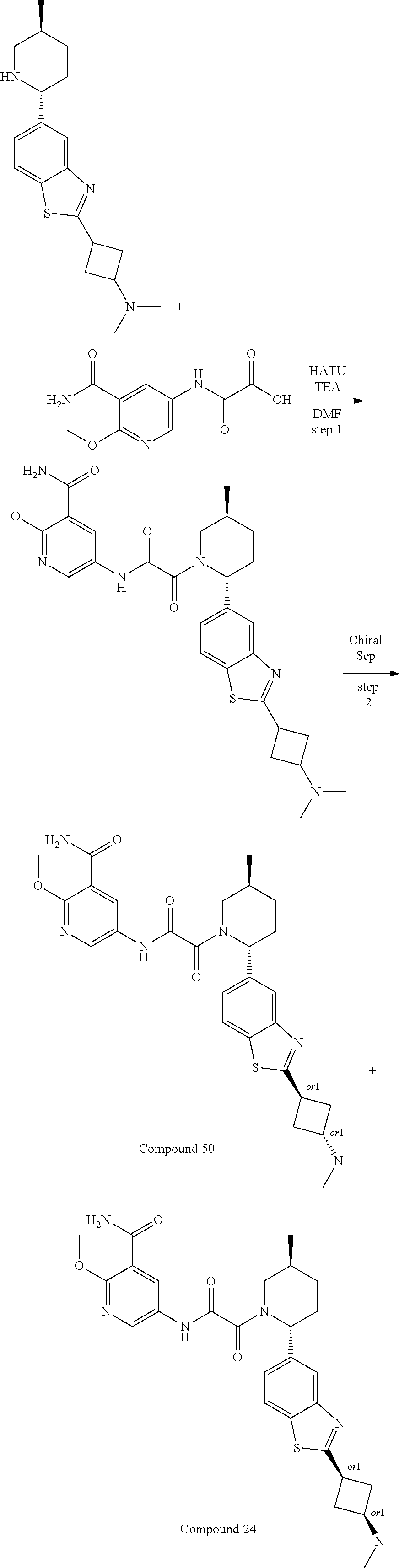

The synthesis of N,N-dimethyl-3-(5-((2R,5S)-5-methylpiperidin-2-yl)benzo[d]thiazol-2-yl)cyclobutanamine is given by 3LLL.

### Step 1: Synthesis of 5-(2-((2R,5S)-2-(2-(3-(dimethylamino)cyclobutyl)benzo[d]thiazol-5-yl)-5-methylpiperidin-1-yl)-2-oxoacetamido)-2-methoxynicotinamide

Prepared by general procedure scheme 4.1 step 6A. Yield: 97 mg (30.76%).

HPLC conditions: Column: XBridge BEH C18 100*19 mm, 5 microM; 0-1-6 min 30-30-80% water-MeOH+0.1% NH₄OH, flow: 30 ml/min; (loading pump 4 ml/min MeCN).

LCMS(ESI): [M]⁺ m/z: calcd 550.2; found 551.2; Rt=2.666 min.

### Step 2: Chiral Separation (Compound 50 and Compound 24)

Racemic 5-(2-((2R,5S)-2-(2-(3-(dimethylamino)cyclobutyl)benzo[d]thiazol-5-yl)-5-methylpiperidin-1-yl)-2-oxoacetamido)-2-methoxynicotinamide (97 mg, 176.5 umol) was chiral separated (Column: Chiralcel OD-H (250*20 mm, 5 mkm); Mobile phase: Hexane-IPA-MeOH, 70-15-15. Flow Rate: 15 mL/min) to obtain 5-(2-((2R,5S)-2-(2-((1r,3R)-3-(dimethylamino)cyclobutyl)benzo[d]thiazol-5-yl)-5-methylpiperidin-1-yl)-2-oxoacetamido)-2-methoxynicotinamide (27 mg, 49.12 mol, 55.67% yield) (RT=37.21 min) and 5-(2-((2R,5S)-2-(2-((1s,3S)-3-(dimethylamino)cyclobutyl)benzo[d]thiazol-5-yl)-5-methylpiperidin-1-yl)-2-oxoacetamido)-2-methoxynicotinamide (30 mg, 54.58 μmol, 61.86% yield) (RT=24.53 min).

Rel Time for Compound 50 in analytical conditions (column: OD-H, Hexane-IPA-MeOH, 50-25-25, 0.6 ml/min as mobile phase) 15.57 min and for Compound 24 13.05 min.

### Compound 50: Retention Time: 15.57 Min

1H NMR (DMSO-d6, 600 MHz): δ (ppm) 1.04 (m, 3H), 1.36 (m, 1H), 1.71 (m, 1H), 1.88 (m, 1H), 2.05 (s, 6H), 2.12 (m, 2H), 2.26 (m, 2H), 2.67 (m, 4H), 3.82 (m, 5H), 5.49 (m, 1H), 7.38 (m, 1H), 7.72 (m, 2H), 7.88 (m, 1H), 8.04 (m, 1H), 8.51 (m, 2H), 11.07 (m, 1H) LCMS(ESI): [M]⁺ m/z: calcd 550.2; found 551.2; Rt=0.955 min.

### Compound 24: Retention Time: 13.05 Min

1H NMR (DMSO-d6, 600 MHz): δ (ppm) 1.04 (m, 3H), 1.37 (m, 1H), 1.72 (m, 1H), 1.89 (m, 1H), 2.06 (m, 7H), 2.32 (m, 1H), 2.40 (m, 5H), 2.91 (m, 1H), 3.79 (m, 5H), 5.50 (m, 1H), 7.38 (m, 1H), 7.73 (m, 2H), 7.90 (m, 1H), 8.04 (m, 1H), 8.51 (m, 2H), 11.08 (m, 1H)

LCMS(ESI): [M]⁺ m/z: calcd 550.2; found 551.2; Rt=0.845 min.

### Example 40. Compound 20 2-methoxy-5-(2-((2R,5S)-5-methyl-2-(2-((1-methylpiperidin-4-yl)methyl)benzo[d]thiazol-5-yl)piperidin-1-yl)-2-oxoacetamido)nicotinamide

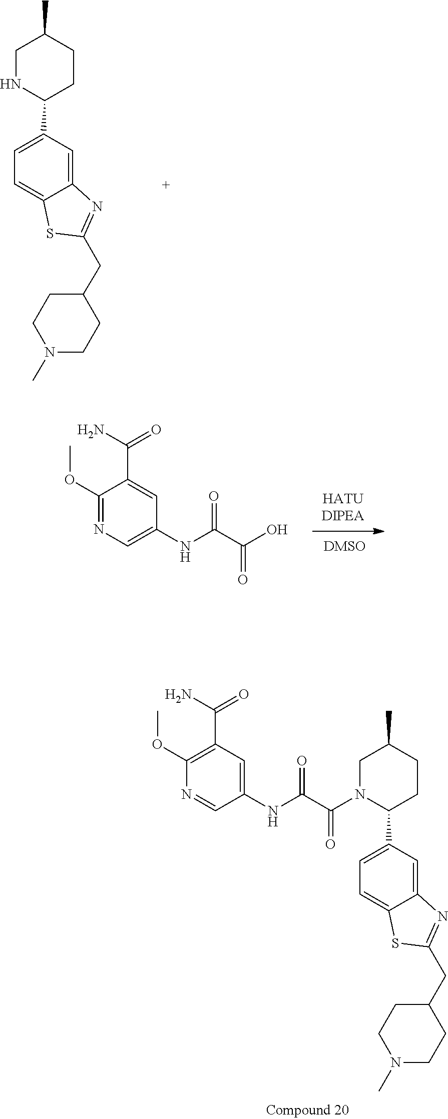

The synthesis of 2-[(1-methyl-4-piperidyl)methyl]-5-[(2R,5S)-5-methyl-2-piperidyl]-1,3-benzothiazole is given by Example 14, steps 1-7.

Prepared by general procedure scheme 4.1 step 6B. Yield: 15 mg (8.44%).

### HPLC Conditions:

First run: Column: SunFire 100*19 mm, 5 microM; 2-10 min 10-40% MeOH+NH₃, flow: 30 ml/min; (loading pump 4 ml/min MeOH+NH₃).

Second run: Column: SunFire 100*19 mm, 5 microM; 2-10 min 10-40% MeCN+FA, flow: 30 ml/min; (loading pump 4 ml/min MeCN).

### Example 41. Compound 98 5-(2-((2R,5S)-2-(2-((S)-2-(dimethylamino)propyl)benzo[d]thiazol-5-yl)-5-methylpiperidin-1-yl)-2-oxoacetamido)-2-methoxynicotinamide

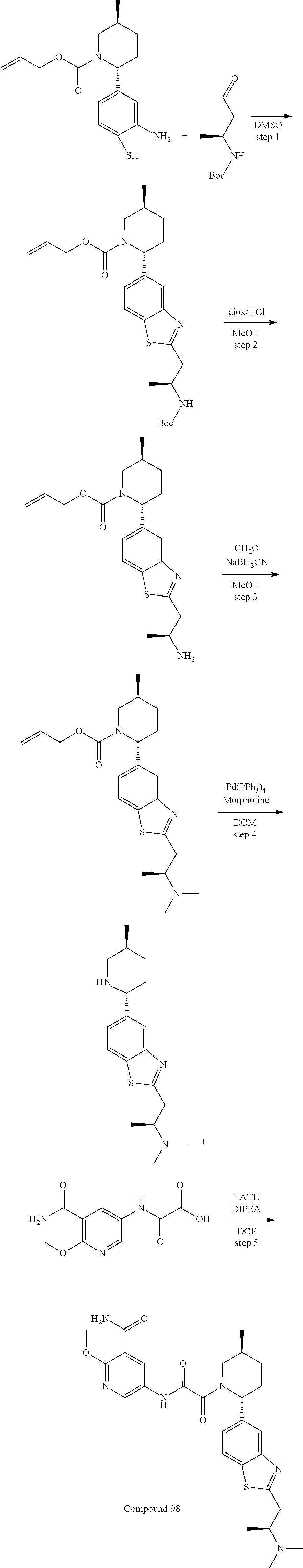

The synthesis of(S)—N,N-dimethyl-1-(5-((2R,5S)-5-methylpiperidin-2-yl)benzo[d]thiazol-2-yl)propan-2-amine is given by 3GGG.

### Step 5: Synthesis of 5-(2-((2R,5S)-2-(2-((S)-2-(dimethylamino)propyl)benzo[d]thiazol-5-yl)-5-methylpiperidin-1-yl)-2-oxoacetamido)-2-methoxynicotinamide (Compound 98)

Prepared by general procedure scheme 4.1 step 6B. Yield: 59 mg (17.39%).

HPLC conditions: Column: XBridge C18 100*19 mm, 5 microM; 0-5 min 30-80% water-MeOH+0.1% NH₄OH, flow: 30 ml/min; (loading pump 4 ml/min MeOH).

Compound 98: ¹H NMR (600 MHz, DMSO-d₆) δ (ppm) 1.02 (m, 6H), 1.36 (m, 1H), 1.71 (m, 1H), 1.88 (m, 1H), 2.08 (m, 1H), 2.21 (m, 6H), 2.30 (m, 1H), 3.03 (m, 3H), 3.21 (m, 1H), 3.79 (m, 4H), 5.49 (m, 1H), 7.36 (m, 1H), 7.72 (m, 2H), 7.85 (m, 1H), 8.01 (m, 1H), 8.50 (m, 2H), 11.08 (m, 1H).

LCMS(ESI): [M]⁺ m/z: calcd 538.2; found 539.2; Rt=2.343 min.

### Example 42. Compound 99 2-methoxy-5-(2-((2R,5S)-5-methyl-2-(2-(2-methyl-2-azabicyclo[2.2.2]octan-4-yl)benzo[d]thiazol-5-yl)piperidin-1-yl)-2-oxoacetamido)nicotinamide

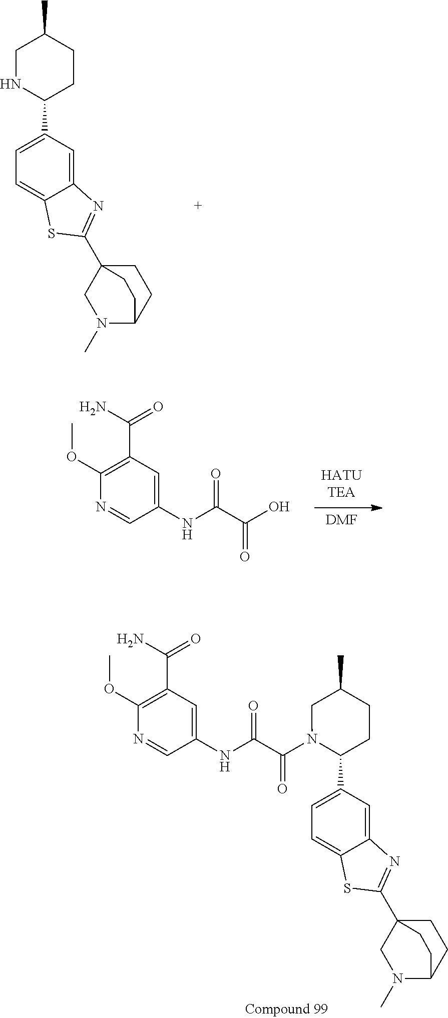

The synthesis of 2-(2-methyl-2-azabicyclo[2.2.2]octan-4-yl)-5-((2R,5S)-5-methylpiperidin-2-yl)benzo[d]thiazole is given by 3MMM.

Prepared by general procedure scheme 4.1 step 6A. Yield: 108 mg (33.29%).

HPLC conditions:

First run: Column: XBridge C18 100×19 mm, 5 microM; 0-5 min 45-95% water-MeOH+0.1% NH₄OH, flow: 30 ml/min; (loading pump 4 ml/min MeOH).

Second run: Column: XBridge C18 100×19 mm, 5 microM; 0-5 min 30-55% water-MeCN+0.1% NH₄OH, flow: 30 ml/min; (loading pump 4 ml/min MeCN).

Compound 99: 1H NMR (DMSO-d6, 600 MHz): δ (ppm) 1.03 (m, 3H), 1.36 (m, 1H), 1.58 (m, 2H), 1.70 (m, 1H), 1.96 (m, 7H), 2.15 (m, 1H), 2.30 (m, 3H), 2.58 (m, 2H), 2.91 (m, 3H), 3.94 (m, 4H), 5.49 (m, 1H), 7.38 (m, 1H), 7.73 (m, 2H), 7.88 (m, 1H), 8.05 (m, 1H), 8.49 (m, 2H), 11.07 (m, 1H)

LCMS(ESI): [M]⁺ m/z: calcd 576.2; found 577.2; Rt=2.532 min.

### Example 43. Compound 28 N-(6-amino-5-ethylpyridin-3-yl)-2-((2R,5S)-5-methyl-2-(2-(1-methyl-1,2,3,6-tetrahydropyridin-4-yl)benzo[d]thiazol-5-yl)piperidin-1-yl)-2-oxoacetamide

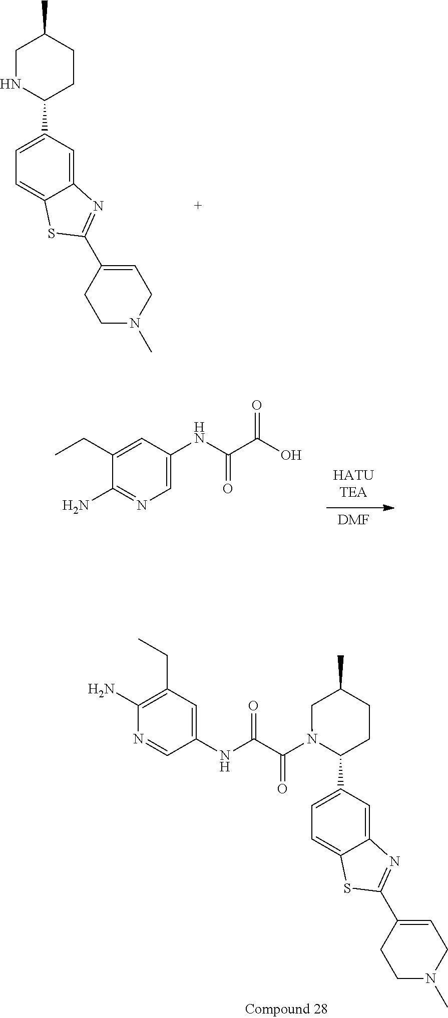

Synthesis of 2-(1-methyl-1,2,3,6-tetrahydropyridin-4-yl)-5-((2R,5S)-5-methylpiperidin-2-yl)benzo[d]thiazole is given by 3NNN.

Prepared by general procedure scheme 4.1 step 6A. Yield: 98 mg (30.94%).

HPLC conditions: Column: SunFire C18 100*19 mm, 5 microM; 2-10 min 35-50% water-MeOH+NH₃; (loading pump 4 ml/min MeOH+NH₃).

Compound 28: ¹H NMR (600 MHz, DMSO-d₆) δ (ppm) 1.03 (m, 3H), 1.12 (m, 3H), 1.36 (m, 1H), 1.71 (m, 1H), 1.88 (m, 1H), 2.08 (m, 1H), 2.18 (m, 1H), 2.29 (s, 3H), 2.34 (d, 2H), 2.41 (m, 2H), 2.58 (m, 2H), 2.67 (m, 2H), 2.83 (m, 1H), 3.76 (dd, 1H), 5.64 (m, 3H), 6.75 (s, 1H), 7.41 (m, 2H), 7.88 (m, 1H), 8.04 (m, 2H), 10.55 (m, 1H).

LCMS(ESI): [M]⁺ m/z: calcd 518.2; found 519.2; Rt=2.004 min.

### Example 44. Compound 21 2-((2R,5S)-2-(2-(1-azabicyclo[2.2.1]heptan-3-yl)benzo[d]thiazol-5-yl)-5-methylpiperidin-1-yl)-N-(6-amino-5-ethylpyridin-3-yl)-2-oxoacetamide

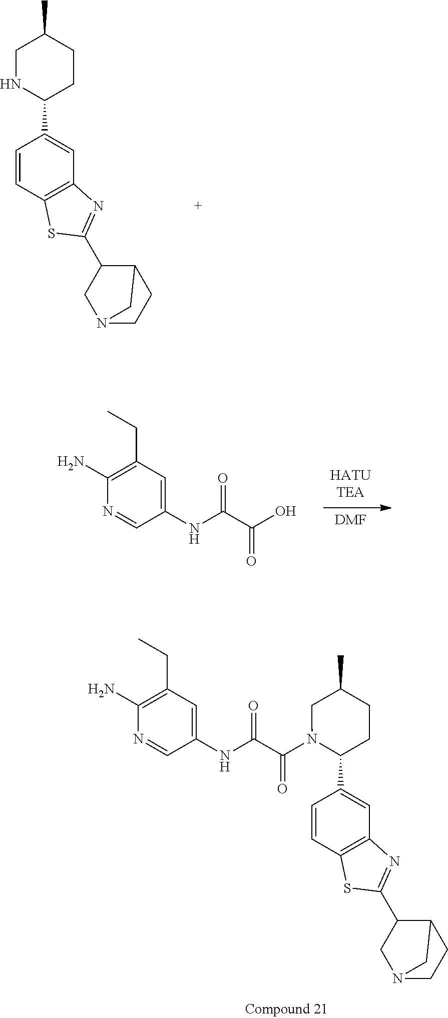

Synthesis of 2-(1-azabicyclo[2.2.1]heptan-3-yl)-5-((2R,5S)-5-methylpiperidin-2-yl)benzo[d]thiazole is given by 3QQQ.

Prepared by general procedure scheme 4.1 step 6A. Yield: 31.3 mg (12.38%).

HPLC conditions: Column: XBridge C18 100*19 mm, 5 microM; 0-5 min 40-90% water-MeOH+0.1% NH₄OH; (loading pump 4 ml/min MeCN).

Compound 21: ¹H NMR (600 MHz, DMSO-d₆) δ (ppm) 0.73-1.04 (m, 3H), 1.06-1.17 (m, 3H), 1.25-1.39 (m, 2H), 1.54-1.65 (m, 1H), 1.65-1.77 (m, 1H), 1.81-1.93 (m, 1H), 2.03-2.22 (m, 1H), 2.23-2.35 (m, 2H), 2.39-2.46 (m, 2H), 2.60-2.71 (m, 2H), 2.71-2.94 (m, 2H), 2.94-3.04 (m, 1H), 3.04-3.18 (m, 2H), 3.40-4.09 (m, 2H), 5.23-5.61 (m, 1H), 5.60-5.80 (m, 2H), 7.31-7.42 (m, 1H), 7.42-7.56 (m, 1H), 7.80-7.94 (m, 1H), 7.95-8.14 (m, 2H), 10.55 (br s, 1H).

LCMS(ESI): [M]⁺ m/z: calcd 518.2; found 519.2; Rt=2.129 min.

### Example 45. Compound 100 N-(6-amino-5-ethylpyridin-3-yl)-2-((2R,5S)-5-methyl-2-(2-(3-(1-methylazetidin-3-yl)cyclobutyl)benzo[d]thiazol-5-yl)piperidin-1-yl)-2-oxoacetamide

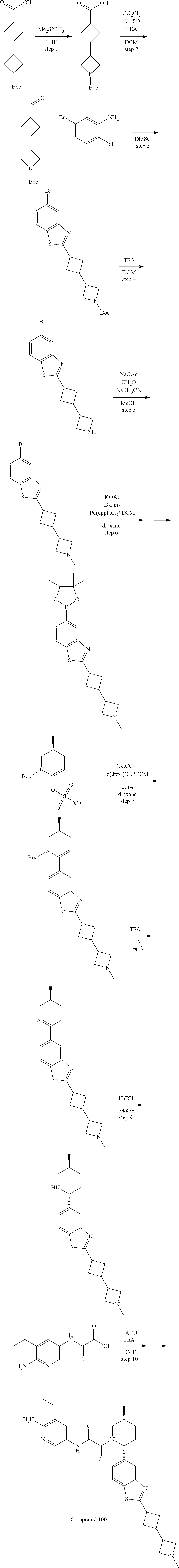

### Step 1: Synthesis of tert-butyl 3-(3-(hydroxymethyl)cyclobutyl)azetidine-1-carboxylate

Borane dimethyl sulfide complex (669.50 mg, 8.81 mmol, 835.83 μL) was added dropwise to the solution of 3-(1-tert-butoxycarbonylazetidin-3-yl)cyclobutanecarboxylic acid (1.5 g, 5.88 mmol) in THF (25 mL) under argon. Resulting mixture was stirred at 20° C. for 18 hr. Then, reaction was quenched by dropwise addition of MeOH (10 ml). When H₂ evolution ceased, volatiles were removed under reduced pressure, leaving tert-butyl 3-[3-(hydroxymethyl)cyclobutyl]azetidine-1-carboxylate (1.42 g, 5.88 mmol, 100.00% yield).

LCMS(ESI): [M-Boc]⁺ m/z: calcd 141.2; found 142.2; Rt=1.205 min.

### Step 2: Synthesis of tert-butyl 3-(3-formylcyclobutyl)azetidine-1-carboxylate

Dimethyl sulfoxide (866.08 mg, 11.08 mmol, 786.63 μL) solution in DCM (10 mL) was added dropwise to the solution of oxalyl chloride (844.15 mg, 6.65 mmol, 580.18 μL) in DCM (10 mL) at −75° C. After addition was complete, stirring was continued for 15 min before tert-butyl 3-[3-(hydroxymethyl)cyclobutyl]azetidine-1-carboxylate (1.07 g, 4.43 mmol) solution in DCM (10 mL) was added thereto dropwise. Resulting solution was stirred at −75° C. for 1 hr. Then, TEA (2.24 g, 22.17 mmol, 3.09 mL) was added dropwise while keeping the internal temperature below −60° C. After 30 min cooling bath was removed and reaction mixture was stirred at ambient temperature for 16 hr. Then, volatiles were removed under reduced pressure, and residue was extracted with hexane (50 ml). Insoluble solids were filtered off and filtrate was concentrated under reduced pressure, leaving tert-butyl 3-(3-formylcyclobutyl)azetidine-1-carboxylate (1.05 g, 4.39 mmol, 98.96% yield).

¹H NMR (500 MHz, CDCl₃) δ (ppm) 1.42 (s, 9H), 1.89 (m, 2H), 2.19 (m, 1H), 2.38 (m, 1H), 2.60 (m, 2H), 3.08 (m, 1H), 3.52 (m, 2H), 3.93 (m, 2H), 9.75 (d, 1H) Step 3: Synthesis of tert-butyl 3-(3-(5-bromobenzo[d]thiazol-2-yl)cyclobutyl)azetidine-1-carboxylate

Prepared by general procedure scheme 4.1 step 1B. Yield: 1.68 g of crude.

LCMS(ESI): [M]⁺ m/z: calcd 423.2; found 424.2; Rt=1.637 min.

### Step 4: Synthesis of 2-(3-(azetidin-3-yl)cyclobutyl)-5-bromobenzo[d]thiazole

TFA (4.52 g, 39.68 mmol, 3.06 mL) was added to the solution of tert-butyl 3-[3-(5-bromo-1,3-benzothiazol-2-yl)cyclobutyl]azetidine-1-carboxylate (1.68 g, 3.97 mmol) in DCM (15 mL). Resulting solution was stirred at 20° C. for 5 hr. Then, volatiles were removed under reduced pressure, leaving 2-[3-(azetidin-3-yl)cyclobutyl]-5-bromo-1,3-benzothiazole (1.75 g, crude, TFA).

LCMS(ESI): [M]⁺ m/z: calcd 323.2; found 324.2; Rt=0.813 min.

### Step 5: Synthesis of 5-bromo-2-(3-(1-methylazetidin-3-yl)cyclobutyl)benzo[d]thiazole

Sodium acetate (1.97 g, 24.07 mmol, 1.29 mL) and formaldehyde, 37% w/w aq. soln., stab. with 7-8% MeOH (651.05 mg, 8.02 mmol, 601.15 μL, 37% purity) were added to the stirred solution of 2-[3-(azetidin-3-yl)cyclobutyl]-5-bromo-1,3-benzothiazole (1.75 g, 4.01 mmol, TFA) in MeOH (30 mL). After 1 hr, sodium cyan borohydride (504.16 mg, 8.02 mmol) was added thereto and resulting mixture was stirred at 20° C. for 15 hr. Then, solvent was removed under reduced pressure and residue was partitioned between 15% aq. K₂CO₃ soln. (30 ml) and DCM (30 ml). Organic layer was separated, dried over solid K₂CO₃ and concentrated under reduced pressure, affording 5-bromo-2-[3-(1-methylazetidin-3-yl)cyclobutyl]-1,3-benzothiazole (1.27 g, 3.77 mmol, 93.87% yield).

LCMS(ESI): [M]⁺ m/z: calcd 337.2; found 338.2; Rt=0.961 min.

### Step 6: Synthesis of 2-(3-(1-methylazetidin-3-yl)cyclobutyl)-5-(4,4,5,5-tetramethyl-1,3,2-dioxaborolan-2-yl)benzo[d]thiazole

Prepared by general procedure scheme 4.1 step 2. Yield: 1.8 g of crude.

LCMS(ESI): [M]⁺ m/z: calcd 384.2; found 385.2; Rt=1.202 min.

### Step 7: Synthesis of (S)-tert-butyl 3-methyl-6-(2-(3-(1-methylazetidin-3-yl)cyclobutyl)benzo[d]thiazol-5-yl)-3,4-dihydropyridine-1(2H)-carboxylate

Prepared by general procedure scheme 4.1 step 3. Yield: 2.4 g of crude.

LCMS(ESI): [M]⁺ m/z: calcd 453.2; found 454.2; Rt=1.277 min.

### Step 8: Synthesis of (S)-5-(5-methyl-3,4,5,6-tetrahydropyridin-2-yl)-2-(3-(1-methylazetidin-3-yl)cyclobutyl)benzo[d]thiazole

Prepared by general procedure scheme 4.1 step 4. Yield: 1.26 g of crude.

LCMS(ESI): [M]⁺ m/z: calcd 353.2; found 354.2; Rt=0.729 min.

### Step 9: Synthesis of 2-(3-(1-methylazetidin-3-yl)cyclobutyl)-5-((2R,5S)-5-methylpiperidin-2-yl)benzo[d]thiazole

Prepared by general procedure scheme 4.1 step 5. Yield: 1.15 g of crude.

LCMS(ESI): [M]⁺ m/z: calcd 355.2; found 356.2; Rt=0.732 min.

### Step 10: Synthesis of N-(6-amino-5-ethylpyridin-3-yl)-2-((2R,5S)-5-methyl-2-(2-(3-(1-methylazetidin-3-yl)cyclobutyl)benzo[d]thiazol-5-yl)piperidin-1-yl)-2-oxoacetamide (Compound 100)

Prepared by general procedure scheme 4.1 step 6A. Yield: 20 mg (13.55%).

HPLC conditions: Column: XBridge C18 100*19 mm, 5 microM; 0-5 min 40-90% water-MeOH+0.1% NH₄OH, flow: 30 ml/min; (loading pump 4 ml/min MeOH).

Compound 100: ¹H NMR (600 MHz, DMSO-d₆) δ (ppm) 1.00-1.14 (m, 6H), 1.30-1.41 (m, 1H), 1.65-1.76 (m, 1H), 1.82-1.93 (m, 1H), 1.95-2.12 (m, 2H), 2.13-2.26 (m, 4H), 2.27-2.36 (m, 2H), 2.38-2.43 (m, 2H), 2.54-2.58 (m, 1H), 2.61-2.87 (m, 3H), 2.93-3.25 (m, 3H), 3.38-4.08 (m, 3H), 5.24-5.73 (m, 3H), 7.31-7.54 (m, 2H), 7.83-7.93 (m, 1H), 7.95-8.09 (m, 2H), 10.49-10.60 (m, 1H).

LCMS(ESI): [M]⁺ m/z: calcd 546.2; found 547.2; Rt=2.218 min.

### Example 46. Compound 87 2-methoxy-5-(2-((2R,5S)-5-methyl-2-(2-(rac-(S)-1,2,2-trimethylpyrrolidin-3-yl)benzo[d]thiazol-5-yl)piperidin-1-yl)-2-oxoacetamido)nicotinamide

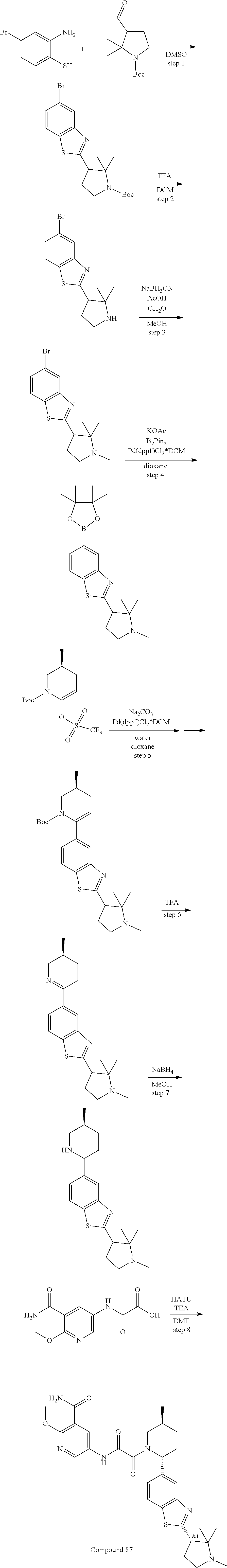

### Step 1: Synthesis of tert-butyl 3-(5-bromobenzo[d]thiazol-2-yl)-2,2-dimethylpyrrolidine-1-carboxylate

Prepared by general procedure scheme 4.1 step 1B. Yield: 5.96 g of crude.

LCMS(ESI): [M-Boc]⁺ m/z: calcd 311.2; found 312.2; Rt=1.773 min.

### Step 2: Synthesis of 5-bromo-2-(2,2-dimethylpyrrolidin-3-yl)benzo[d]thiazole

TFA (16.52 g, 144.89 mmol, 11.16 mL) was added in one portion to a stirred solution of tert-butyl 3-(5-bromo-1,3-benzothiazol-2-yl)-2,2-dimethyl-pyrrolidine-1-carboxylate (5.96 g, 14.49 mmol) in DCM (15 mL) at 25° C. The resulting solution was stirred at 25° C. for 2 hr, and then concentrated in vacuum. The residue was diluted with ice cold water (50 ml) and basified to pH 11 with 10% aqueous sodium hydroxide solution. The resulting mixture was extracted with DCM (2*75 ml). The combined organic extracts were dried over sodium sulphate and concentrated in vacuum to afford crude 5-bromo-2-(2,2-dimethylpyrrolidin-3-yl)-1,3-benzothiazole (4.16 g, crude) as red gum, which was used directly in the next step.

LCMS(ESI): [M]⁺ m/z: calcd 311.2; found 312.2; Rt=0.955 min.

### Step 3: Synthesis of 5-bromo-2-(1,2,2-trimethylpyrrolidin-3-yl)benzo[d]thiazole

Formaldehyde, 37% w/w aq. soln., stab. with 7-8% MeOH (1.55 g, 19.05 mmol, 1.43 mL, 37% purity) and acetic acid (1.55 g, 25.75 mmol, 1.47 mL) were added to a stirred solution of 5-bromo-2-(2,2-dimethylpyrrolidin-3-yl)-1,3-benzothiazole (3.66 g, 11.76 mmol) in MeOH (119.10 mL) at 25° C. The resulting mixture was stirred at 25° C. for 0.5 hr, then sodium cyan borohydride (1.48 g, 23.52 mmol) was added in one portion at 25° C. (foaming!). The reaction mixture was stirred at 25° C. for 12 hr, and then concentrated in vacuum. The residue was diluted with 10% aqueous sodium hydroxide solution (20 ml) and extracted with DCM (2*20 ml). The combined organic extracts were dried over sodium sulphate and concentrated in vacuum to afford crude 5-bromo-2-(1,2,2-trimethylpyrrolidin-3-yl)-1,3-benzothiazole (3.35 g, crude) as light-yellow gum, which was used directly in the next step.

LCMS(ESI): [M]⁺ m/z: calcd 325.2; found 326.2; Rt=0.984 min.

### Step 4: Synthesis of 5-(4,4,5,5-tetramethyl-1,3,2-dioxaborolan-2-yl)-2-(1,2,2-trimethylpyrrolidin-3-yl)benzo[d]thiazole

Prepared by general procedure scheme 4.1 step 2. Yield: 2.98 g of crude.

LCMS(ESI): [M]⁺ m/z: calcd 372.2; found 373.2; Rt=1.146 min.

### Step 5: Synthesis of (3S)-tert-butyl 3-methyl-6-(2-(1,2,2-trimethylpyrrolidin-3-yl)benzo[d]thiazol-5-yl)-3,4-dihydropyridine-1(2H)-carboxylate

Prepared by general procedure scheme 4.1 step 3. Yield: 3.1 g of crude.

LCMS(ESI): [M]⁺ m/z: calcd 441.2; found 442.2; Rt=1.137 min.

### Step 6: Synthesis of 5-((S)-5-methyl-3,4,5,6-tetrahydropyridin-2-yl)-2-(1,2,2-trimethylpyrrolidin-3-yl)benzo[d]thiazole

Prepared by general procedure scheme 4.1 step 4. Yield: 1.8 g of crude.

LCMS(ESI): [M]⁺ m/z: calcd 341.2; found 342.2; Rt=0.487 min.

### Step 7: Synthesis of 5-((2R,5S)-5-methylpiperidin-2-yl)-2-(1,2,2-trimethylpyrrolidin-3-yl)benzo[d]thiazole

Prepared by general procedure scheme 4.1 step 5. Yield: 1.08 g of crude.

LCMS(ESI): [M]⁺ m/z: calcd 343.2; found 344.2; Rt=0.531 min.

### Step 8: Synthesis of 2-methoxy-5-(2-((2R,5S)-5-methyl-2-(2-(rac-(S)-1,2,2-trimethylpyrrolidin-3-yl)benzo[d]thiazol-5-yl)piperidin-1-yl)-2-oxoacetamido)nicotinamide

Prepared by general procedure scheme 4.1 step 6A. Yield: 78.2 mg (21.14%).

HPLC conditions: Column: SunFire C18 100*19 mm, 5 microM; 2-10 min 30% water-MeCN+FA, flow: 30 ml/min; (loading pump 4 ml/min MeCN).

Compound 87: ¹H NMR (600 MHz, DMSO-d₆) δ (ppm) 0.62-0.70 (m, 3H), 0.98-1.06 (m, 3H), 1.13-1.18 (m, 3H), 1.31-1.44 (m, 1H), 1.65-1.79 (m, 1H), 1.82-1.97 (m, 1H), 2.05-2.17 (m, 1H), 2.17-2.22 (m, 4H), 2.24-2.36 (m, 2H), 2.73-2.81 (m, 1H), 2.82-2.87 (m, 1H), 3.41-3.46 (m, 2H), 3.49-3.53 (m, 0.7H), 3.89-3.99 (m, 3H), 4.02-4.06 (m, 0.3H), 5.24-5.73 (m, 1H), 7.30-7.45 (m, 1H), 7.60-7.78 (m, 2H), 7.85-7.94 (m, 1H), 7.98-8.09 (m, 1H), 8.39-8.59 (m, 2H), 10.97-11.15 (m, 1H).

LCMS(ESI): [M]⁺ m/z: calcd 564.2; found 565.2; Rt=2.003 min.

### Example 47. Compound 83 2-methoxy-5-(2-((2R,5S)-5-methyl-2-(2-(1-methyl-1,2,3,6-tetrahydropyridin-4-yl)benzo[d]thiazol-5-yl)piperidin-1-yl)-2-oxoacetamido)nicotinamide

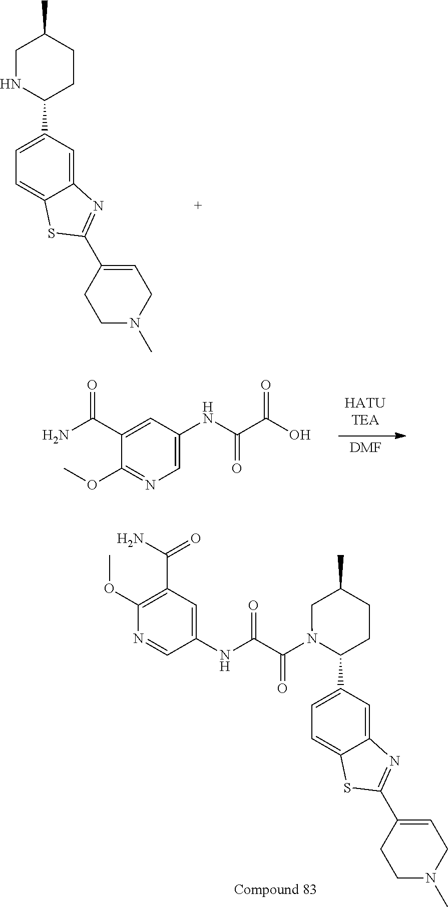

The synthesis of 2-(1-methyl-1,2,3,6-tetrahydropyridin-4-yl)-5-((2R,5S)-5-methylpiperidin-2-yl)benzo[d]thiazole is given by 3JJJ.

HATU (801.13 mg, 2.11 mmol) was added in small portions over 0.5 hr period to a stirred mixture of 2-(1-methyl-3,6-dihydro-2H-pyridin-4-yl)-5-[(2R,5S)-5-methyl-2-piperidyl]-1,3-benzothiazole (600 mg, 1.83 mmol), 2-[(5-carbamoyl-6-methoxy-3-pyridyl)amino]-2-oxo-acetic acid (547.77 mg, 2.29 mmol) and TEA (1.11 g, 10.99 mmol, 1.53 mL) in DMF (5 mL) at 25° C. The resulting mixture was stirred at 25° C. for 2 hr, and then submitted to reverse phase HPLC (column: YMC Triart C18 100×20 mm, 5 um; mobile phase:20-20-40% 0-1-6 min H₂O/MeCN/0.1% NH₄OH; flow rate: 30 ml/min (loading pump 4 ml/min MeCN)) to afford 249 mg of the product 87% purity by LCMS, which was purified by preparative chiral HPLC (column: Chiralpak AS-H (250*4.6 mm, 5 mkm); Mobile phase: Hexane-IPA-MeOH, 70-15-15; Flow rate: 14 ml/min) to afford crude product (RT=21.839 min.), which was again purified by preparative chiral HPLC (column: Chiralcel OD-H (250*20, 5 mkm); mobile phase:Hexane-IPA-MeOH, 70-15-15; flow rate: 14 ml/min) to afford Compound 83 2-methoxy-5-[[2-oxo-2-[(2R,5S)-5-methyl-2-[2-(1-methyl-3,6-dihydro-2H-pyridin-4-yl)-1,3-benzothiazol-5-yl]-1-piperidyl]acetyl]amino]pyridine-3-carboxamide (71 mg, 129.41 mol, 7.06% yield) (RT=42.142 min) as light-yellow solid.

Compound 83: ¹H NMR (600 MHz, DMSO-d₆) δ (ppm) 1.01-1.07 (m, 3H), 1.29-1.42 (m, 1H), 1.62-1.78 (m, 1H), 1.83-1.96 (m, 1H), 2.05-2.23 (m, 1H), 2.29 (s, 3H), 2.31-2.37 (m, 1H), 2.56-2.60 (m, 2H), 2.61-2.73 (m, 2H), 2.81-2.86 (m, 0.3H), 3.04-3.18 (m, 2H), 3.18-3.27 (m, 0.7H), 3.49-3.54 (m, 07H), 3.90-3.97 (m, 3H), 4.02-4.06 (m, 0.3H), 5.20-5.78 (m, 1H), 6.71-6.79 (m, 1H), 7.34-7.47 (m, 1H), 7.67-7.76 (m, 2H), 7.83-7.94 (m, 1H), 7.99-8.09 (m, 1H), 8.41-8.59 (m, 2H), 11.00-11.15 (m, 1H).

LCMS(ESI): [M]⁺ m/z: calcd 548.2; found 549.2; Rt=2.643 min.

### Example 48. Compound 84 N-(6-amino-5-ethylpyridin-3-yl)-2-((2R,5S)-2-(2-(1,5-dimethyl-1,2,3,6-tetrahydropyridin-4-yl)benzo[d]thiazol-5-yl)-5-methylpiperidin-1-yl)-2-oxoacetamide

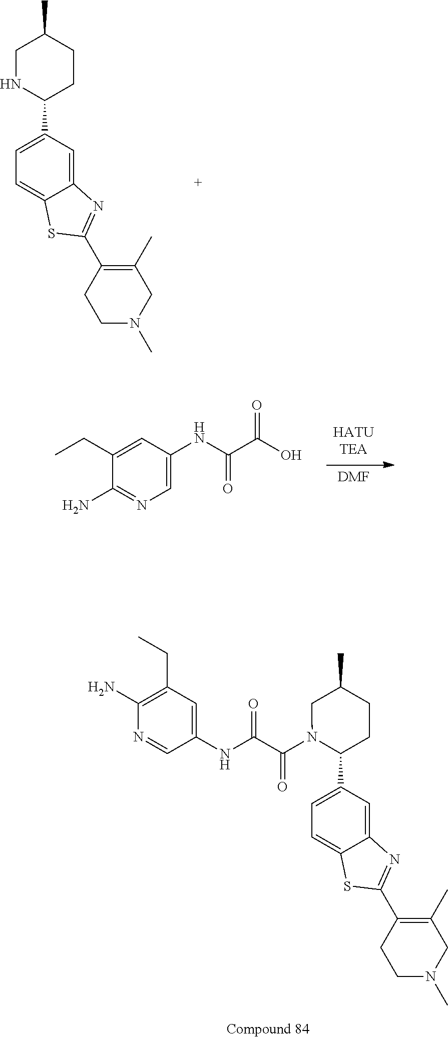

Synthesis of 2-(1,5-dimethyl-1,2,3,6-tetrahydropyridin-4-yl)-5-((2R,5S)-5-methylpiperidin-2-yl)benzo[d]thiazole is given by 3JJJ.

Prepared by general procedure scheme 4.1 step 6A. Yield: 31 mg (9.94%).

HPLC conditions: Column: XBridge C18 100*19 mm, 5 microM; 0-1-5 min 30-30-70% water-MeCN+0.1% NH₄OH; (loading pump 4 ml/min MeCN).

Compound 84: ¹H NMR (600 MHz, DMSO-d₆) δ (ppm) 1.02-1.15 (m, 6H), 1.30-1.42 (m, 1H), 1.68-1.76 (m, 1H), 1.84-1.93 (m, 1H), 2.04-2.24 (m, 4H), 2.24-2.37 (m, 5H), 2.38-2.44 (m, 1H), 2.56-2.71 (m, 5H), 3.04 (s, 2H), 3.47-4.08 (m, 1H), 5.25-5.74 (m, 3H), 7.35-7.57 (m, 2H), 7.87-7.96 (m, 1H), 7.98-8.14 (m, 2H), 10.51-10.63 (m, 1H).

LCMS(ESI): [M]⁺ m/z: calcd 532.2; found 533.2; Rt=0.829 min.

### Example 49. Compound 43, Compound 40 and Compound 119 N-(6-amino-5-ethylpyridin-3-yl)-2-((2R,5S)-2-(2-(3-(dimethylamino)cyclobutyl)benzo[d]thiazol-5-yl)-5-methylpiperidin-1-yl)-2-oxoacetamide

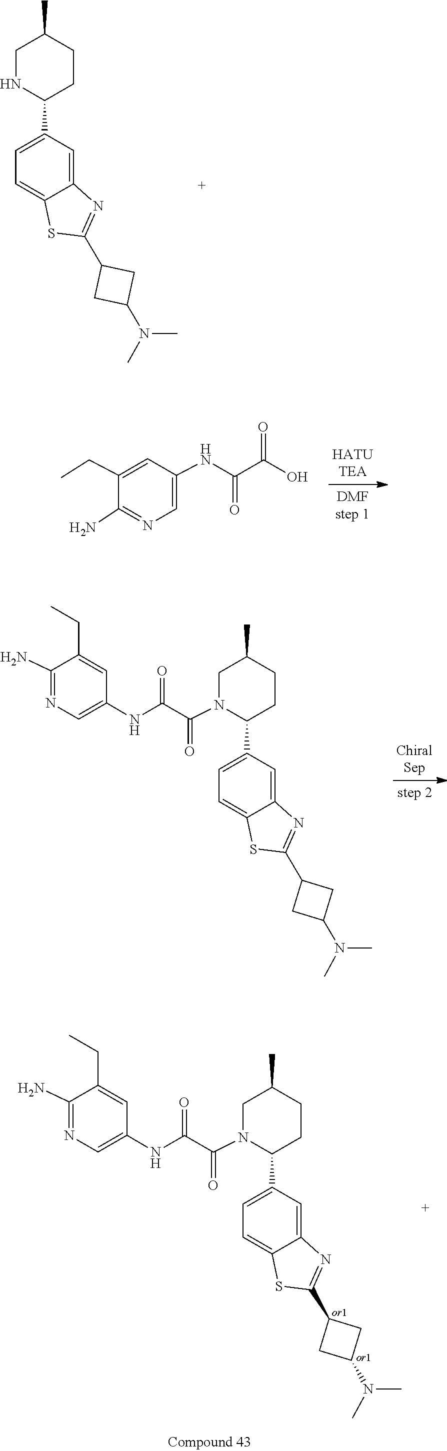 [SMILES: C[C@H]1CC[C@H](c2ccc3sc(C4CC(N(C)C)C4)nc3c2)NC1 ; CCc1cc(NC(=O)C(=O)O)cnc1N ; CCc1cc(NC(=O)C(=O)N2C[C@@H](C)CC[C@@H]2c2ccc3sc(C4CC(N(C)C)C4)nc3c2)cnc1N ; CCc1cc(NC(=O)C(=O)N2C[C@@H](C)CC[C@@H]2c2ccc3sc([C@H]4C[C@H](N(C)C)C4)nc3c2)cnc1N]

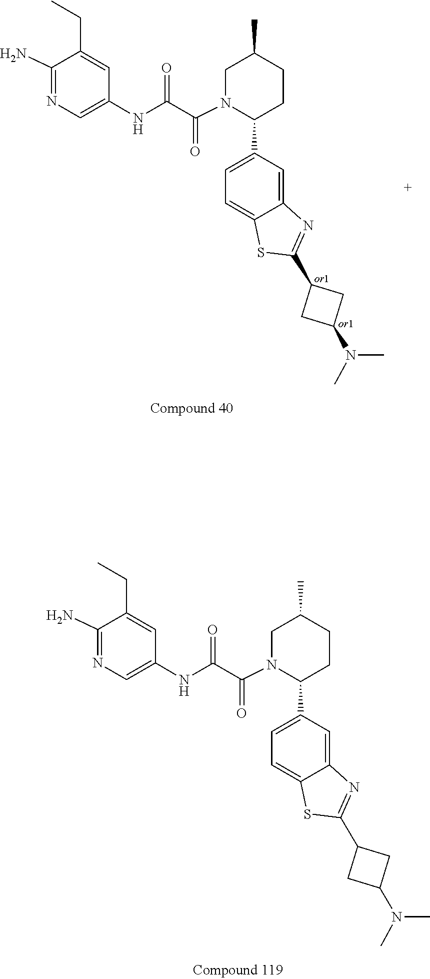 [SMILES: CCc1cc(NC(=O)C(=O)N2C[C@@H](C)CC[C@@H]2c2ccc3sc([C@H]4C[C@@H](N(C)C)C4)nc3c2)cnc1N ; CCc1cc(NC(=O)C(=O)N2C[C@H](C)CC[C@@H]2c2ccc3sc(C4CC(N(C)C)C4)nc3c2)cnc1N]

The synthesis of N,N-dimethyl-3-(5-((2R,5S)-5-methylpiperidin-2-yl)benzo[d]thiazol-2-yl)cyclobutanamine is given by 3LLL.

### Step 1: Synthesis of N-(6-amino-5-ethylpyridin-3-yl)-2-((2R,5S)-2-(2-(3-(dimethylamino)cyclobutyl)benzo[d]thiazol-5-yl)-5-methylpiperidin-1-yl)-2-oxoacetamide

Prepared by general procedure scheme 4.1 step 6A. Yield: 178 mg (29.26%).

HPLC conditions: Column: XBridge BEH C18 100*19 mm, 5 microM; 0-5 min 40-90% water-MeOH+0.1% NH₄OH, flow: 30 ml/min; (loading pump 4 ml/min MeOH).

LCMS(ESI): [M]⁺ m/z: calcd 520.2; found 521.2; Rt=1.857 min.

### Step 2: Chiral Separation (Compound 43, Compound 40 and Compound 119)

Racemic N-(6-amino-5-ethyl-3-pyridyl)-2-oxo-2-[(2R,5S)-2-[2-[3-(dimethylamino)cyclobutyl]-1,3-benzothiazol-5-yl]-5-methyl-1-piperidyl]acetamide (178 mg, 341.86 μmol) was chiral separated (Column: Chiralcel OD-H (250*20, 5 mkm), Hexane-IPA-MeOH, 80-10-10, 12 ml/min) to obtain N-(6-amino-5-ethyl-3-pyridyl)-2-oxo-2-[(2R,5S)-2-[2-[3-(dimethylamino)cyclobutyl]-1,3-benzothiazol-5-yl]-5-methyl-1-piperidyl]acetamide (59 mg, 113.31 mol, 33.15% yield), N-(6-amino-5-ethyl-3-pyridyl)-2-oxo-2-[(2R,5S)-2-[2-[3-(dimethylamino)cyclobutyl]-1,3-benzothiazol-5-yl]-5-methyl-1-piperidyl]acetamide (58 mg, 111.39 μmol, 32.58% yield) and N-(6-amino-5-ethyl-3-pyridyl)-2-oxo-2-[(2R,5R)-2-[2-[3-(dimethylamino)cyclobutyl]-1,3-benzothiazol-5-yl]-5-methyl-1-piperidyl]acetamide (15 mg, 28.81 μmol, 8.43% yield).

Rel Time for Compound 43 in analytical conditions (column: OD-H, Hexane-IPA-MeOH, 70-15-15, 0.6 ml/min as mobile phase) 16.92 min, for Compound 40 19.24 min and for Compound 119 15.67 min.

### Compound 43: Retention Time: 15.57 Min

1H NMR (DMSO-d6, 500 MHz): δ (ppm) 1.08 (m, 6H), 1.34 (m, 1H), 1.70 (m, 1H), 1.86 (m, 1H), 2.05 (s, 6H), 2.40 (m, 7H), 2.91 (m, 2H), 3.83 (m, 3H), 5.64 (m, 3H), 7.41 (m, 2H), 7.90 (m, 1H), 8.04 (m, 2H), 10.55 (m, 1H) LCMS(ESI): [M]⁺ m/z: calcd 520.2; found 521.2; Rt=0.718 min.

### Compound 40: Retention Time: 19.24 Min

1H NMR (DMSO-d6, 500 MHz): δ (ppm) 1.08 (m, 6H), 1.35 (m, 1H), 1.70 (m, 1H), 1.87 (m, 1H), 2.06 (s, 6H), 2.14 (m, 2H), 2.40 (m, 6H), 2.73 (m, 2H), 3.62 (m, 2H), 5.64 (m, 3H), 7.42 (m, 2H), 7.88 (m, 1H), 8.03 (m, 2H), 10.55 (m, 1H)

LCMS(ESI): [M]⁺ m/z: calcd 520.2; found 521.2; Rt=0.713 min.

### Compound 119: Retention Time: 15.67 Min

1H NMR (DMSO-d6, 600 MHz): δ (ppm) 0.75 (m, 3H), 1.10 (m, 3H), 1.68 (m, 2H), 1.93 (m, 1H), 2.05 (m, 6H), 2.13 (m, 2H), 2.40 (m, 4H), 2.57 (m, 3H), 2.75 (m, 1H), 3.88 (m, 2H), 5.68 (m, 3H), 7.37 (m, 1H), 7.49 (m, 1H), 7.89 (m, 1H), 8.04 (m, 2H), 10.60 (m, 1H)

LCMS(ESI): [M]⁺ m/z: calcd 520.2; found 521.2; Rt=0.726 min.

### Example 50. Compound 6 N-(6-amino-5-ethylpyridin-3-yl)-2-((2R,5S)-2-(2-((1,4-dimethylpiperidin-4-yl)methyl)benzo[d]thiazol-5-yl)-5-methylpiperidin-1-yl)-2-oxoacetamide

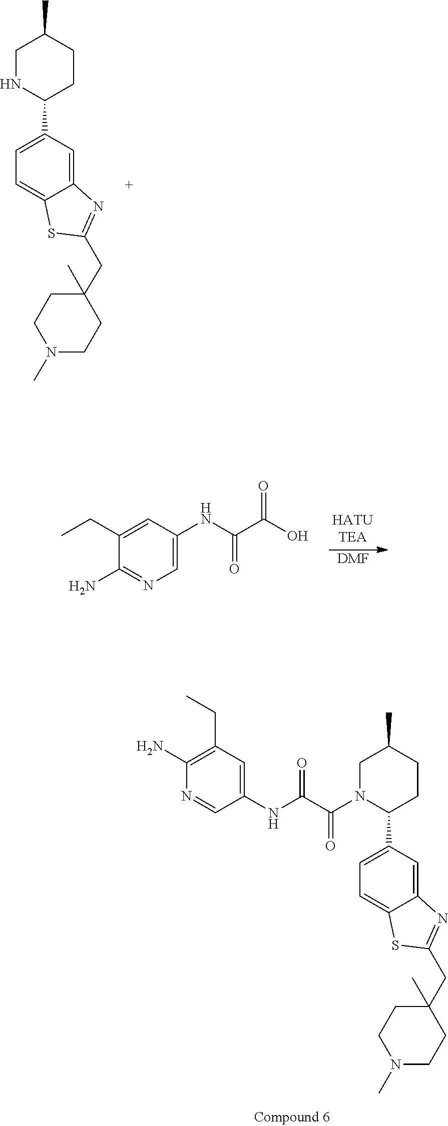

The synthesis of 2-((1,4-dimethylpiperidin-4-yl)methyl)-5-((2R,5S)-5-methylpiperidin-2-yl)benzo[d]thiazole by 3SSS.

Prepared by general procedure scheme 4.1 step 6A. Yield: 83.9 mg (54.67%).

HPLC conditions: Column: SunFire C18 100*19 mm, 5 microM; 0-5 min 50-75% water-MeCN+NH₃; (loading pump 4 ml/min MeCN).

### Compound 6

¹H NMR (600 MHz, DMSO-d₆) δ (ppm) 0.74-1.04 (m, 6H), 1.05-1.14 (m, 3H), 1.31-1.42 (m, 3H), 1.53-1.61 (m, 2H), 1.66-1.74 (m, 1H), 1.81-1.96 (m, 1H), 2.02-2.13 (m, 1H), 2.15 (s, 3H), 2.16-2.30 (m, 3H), 2.31-2.36 (m, 1H), 2.39-2.45 (m, 3H), 2.77-2.82 (m, 0.3H), 3.03 (s, 2H), 3.20-3.28 (m, 0.7H), 3.47-4.09 (m, 1H), 5.25-5.60 (m, 1H), 5.60-5.73 (m, 2H), 7.31-7.43 (m, 1H), 7.43-7.55 (m, 1H), 7.85-7.96 (m, 1H), 7.97-8.11 (m, 2H), 10.41-10.72 (m, 1H).

LCMS(ESI): [M]⁺ m/z: calcd 548.2; found 549.2; Rt=0.955 min.

### Example 51. Compound ent-71 and Compound 71 2-methoxy-5-(2-((2R,5S)-5-methyl-2-(2-(2-methyl-2-azabicyclo[2.2.1]heptan-4-yl)benzo[d]thiazol-5-yl)piperidin-1-yl)-2-oxoacetamido)nicotinamide

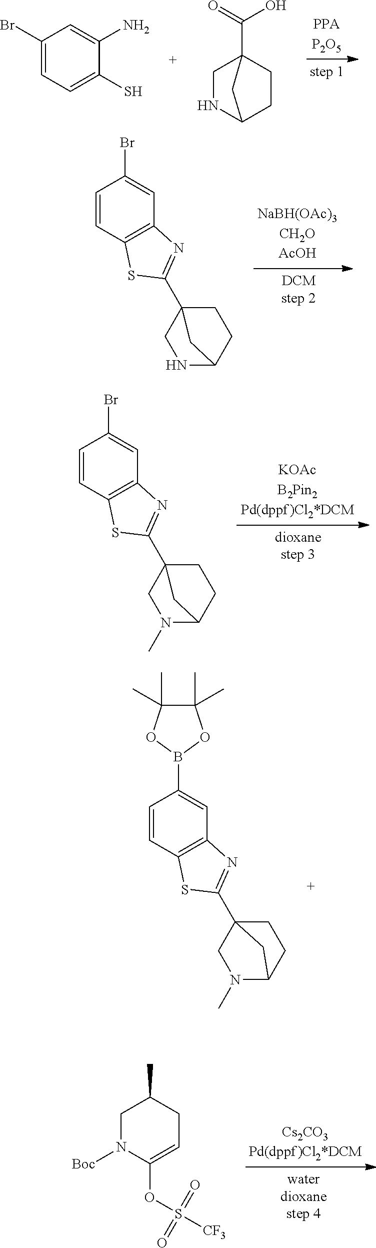

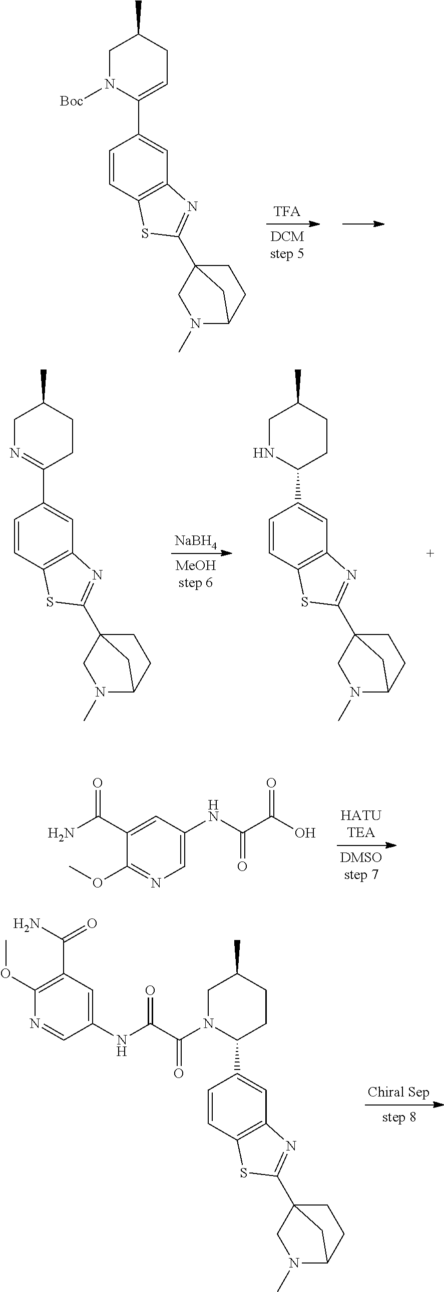

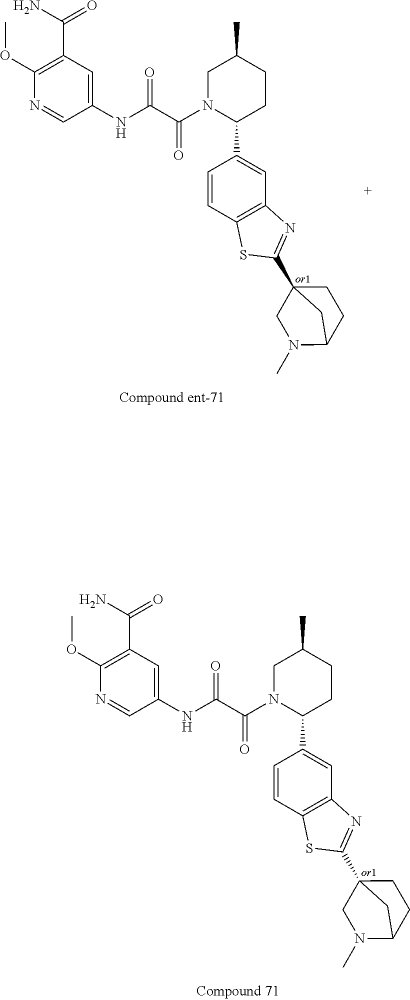 [SMILES: COc1ncc(NC(=O)C(=O)N2C[C@@H](C)CC[C@@H]2c2ccc3sc([C@@]45CCC(C4)N(C)C5)nc3c2)cc1C(N)=O ; COc1ncc(NC(=O)C(=O)N2C[C@@H](C)CC[C@@H]2c2ccc3sc([C@]45CCC(C4)N(C)C5)nc3c2)cc1C(N)=O]

### Step 1: Synthesis of 2-(2-azabicyclo[2.2.1]heptan-4-yl)-5-bromobenzo[d]thiazole

Prepared by general procedure scheme 4.1 step 1A. Yield: 2.57 g (84.81%).

LCMS(ESI): [M]⁺ m/z: calcd 309.2; found 310.2; Rt=0.808 min.

### Step 2: Synthesis of 5-bromo-2-(2-methyl-2-azabicyclo[2.2.1]heptan-4-yl)benzo[d]thiazole

To a stirred solution of 2-(2-azabicyclo[2.2.1]heptan-4-yl)-5-bromo-1,3-benzothiazole (2.57 g, 8.31 mmol) in DCM (150 mL) were added formaldehyde (1.01 g, 12.47 mmol, 934.15 μL, 37% purity) and acetic acid (2.00 g, 33.24 mmol, 1.90 mL) respectively at 25° C. The resulting reaction mixture was stirred at 25° C. for 1 hr. Then sodium bis(acetyloxy)boranuidyl acetate (3.52 g, 16.62 mmol) was added. Mixture was continued stirring for 12 hr. Upon completion, the reaction mixture was quenched with NaHCO₃ solution 50 ml. Then organic phase was washed with water 50 mL, dried over Na₂SO₄ and concentrated under reduced pressure to obtain 5-bromo-2-(2-methyl-2-azabicyclo[2.2.1]heptan-4-yl)-1,3-benzothiazole (2.1 g, 6.50 mmol, 78.17% yield) as brown solid.

LCMS(ESI): [M]⁺ m/z: calcd 323.2; found 324.2; Rt=0.868 min.

### Step 3: Synthesis of 2-(2-methyl-2-azabicyclo[2.2.1]heptan-4-yl)-5-(4,4,5,5-tetramethyl-1,3,2-dioxaborolan-2-yl)benzo[d]thiazole

Prepared by general procedure scheme 4.1 step 2. Yield: 2 g of crude.

LCMS(ESI): [M]⁺ m/z: calcd 370.2; found 371.2; Rt=1.004 min.

### Step 4: Synthesis of (3S)-tert-butyl 3-methyl-6-(2-(2-methyl-2-azabicyclo[2.2.1]heptan-4-yl)benzo[d]thiazol-5-yl)-3,4-dihydropyridine-1(2H)-carboxylate

Prepared by general procedure scheme 4.1 step 3. Yield: 2 g of crude.

LCMS(ESI): [M]⁺ m/z: calcd 439.2; found 440.2; Rt=1.054 min.

### Step 5: Synthesis of 5-((S)-5-methyl-3,4,5,6-tetrahydropyridin-2-yl)-2-(2-methyl-2-azabicyclo[2.2.1]heptan-4-yl)benzo[d]thiazole

Prepared by general procedure scheme 4.1 step 4. Yield: 1.33 g of crude.

LCMS(ESI): [M]⁺ m/z: calcd 339.2; found 340.2; Rt=0.712 min.

### Step 6: Synthesis of 2-(2-methyl-2-azabicyclo[2.2.1]heptan-4-yl)-5-((2R,5S)-5-methylpiperidin-2-yl)benzo[d]thiazole

Prepared by general procedure scheme 4.1 step 5. Yield: 180 mg (67.27%).

HPLC conditions: Column: SunFire C18 100*19 mm, 5 microM; 2-10 min 35-50% water-MeCN+NH₃, flow: 30 ml/min; (loading pump 4 ml/min MeCN).

LCMS(ESI): [M]⁺ m/z: calcd 341.2; found 342.2; Rt=0.638 min.

### Step 7: Synthesis of 2-methoxy-5-(2-((2R,5S)-5-methyl-2-(2-(2-methyl-2-azabicyclo[2.2.1]heptan-4-yl)benzo[d]thiazol-5-yl)piperidin-1-yl)-2-oxoacetamido)

Prepared by general procedure scheme 4.1 step 6A. Yield: 350 mg of crude.

LCMS(ESI): [M]⁺ m/z: calcd 562.2; found 563.2; Rt=0.921 min.

### Step 8: Chiral Separation (Compound ent-71 and Compound 71)

Racemic 2-methoxy-5-(2-((2R,5S)-5-methyl-2-(2-(2-methyl-2-azabicyclo[2.2.1]heptan-4-yl)benzo[d]thiazol-5-yl)piperidin-1-yl)-2-oxoacetamido)nicotinamide (108 mg, 191.94 umol) was chiral separated (Column: Chiralcel OD-H (250×20 mm, 5 mkm) Mobile Phase: Hexane:MeOH:IPA, 70:15:15 Flow Rate: 12 ml/min) to obtain 2-methoxy-5-(2-((2R,5S)-5-methyl-2-(2-((45)-2-methyl-2-azabicyclo[2.2.1]heptan-4-yl)benzo[d]thiazol-5-yl)piperidin-1-yl)-2-oxoacetamido)nicotinamide (31.2 mg, 55.45 μmol) and 2-methoxy-5-(2-((2R,5S)-5-methyl-2-(2-((4R)-2-methyl-2-azabicyclo[2.2.1]heptan-4-yl)benzo[d]thiazol-5-yl)piperidin-1-yl)-2-oxoacetamido)nicotinamide (23.6 mg, 41.94 μmol).

Rel Time for Compound ent-71 in analytical conditions (column: OD-H, Hexane-IPA-MeOH, 70-15-15, 0.6 ml/min as mobile phase) 20.79 min and for Compound 71 24.25 min.

### Compound Ent-71: Retention Time: 20.79 Min

¹H NMR (600 MHz, DMSO-d₆) δ (ppm) 0.84-1.05 (m, 3H), 1.10-1.19 (m, 1H), 1.21-1.24 (m, 3H), 1.29-1.36 (m, 1H), 1.67-1.78 (m, 2H), 1.79-1.95 (m, 4H), 1.99-2.21 (m, 3H), 2.34-2.41 (m, 3H), 2.82-3.17 (m, 1H), 3.48-3.52 (m, 0.7H), 3.91-3.98 (m, 3H), 4.01-4.07 (m, 0.3H), 5.25-5.76 (m, 1H), 7.33-7.49 (m, 1H), 7.66-7.78 (m, 2H), 7.83-7.94 (m, 1H), 8.00-8.14 (m, 1H), 8.38-8.60 (m, 2H), 10.98-11.18 (m, 1H).

LCMS(ESI): [M]⁺ m/z: calcd 562.2; found 563.2; Rt=1.337 min.

### Compound 71: Retention Time: 24.25 Min

¹H NMR (600 MHz, DMSO-d₆) δ (ppm) 0.84-1.05 (m, 3H), 1.12-1.19 (m, 1H), 1.20-1.23 (m, 3H), 1.29-1.39 (m, 1H), 1.64-1.80 (m, 2H), 1.81-1.95 (m, 4H), 2.00-2.22 (m, 3H), 2.33-2.41 (m, 3H), 2.83-3.20 (m, 1H), 3.48-3.54 (m, 0.7H), 3.91-3.98 (m, 3H), 4.01-4.05 (m, 0.3H), 5.26-5.77 (m, 1H), 7.34-7.46 (m, 1H), 7.65-7.80 (m, 2H), 7.84-7.94 (m, 1H), 8.02-8.12 (m, 1H), 8.38-8.66 (m, 2H), 10.90-11.27 (m, 1H).

LCMS(ESI): [M]⁺ m/z: calcd 562.2; found 563.2; Rt=1.339 min.

### Example 52. Compound 10 N-(6-amino-5-ethylpyridin-3-yl)-2-((2R,5S)-2-(2-(rac-(3S,4S)-1,4-dimethylpyrrolidin-3-yl)benzo[d]thiazol-5-yl)-5-methylpiperidin-1-yl)-2-oxoacetamide

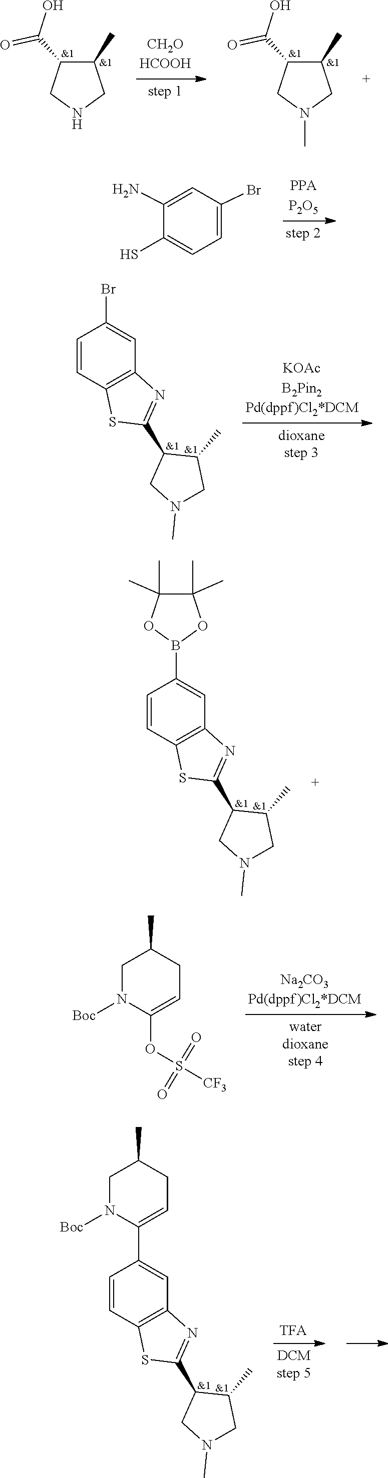

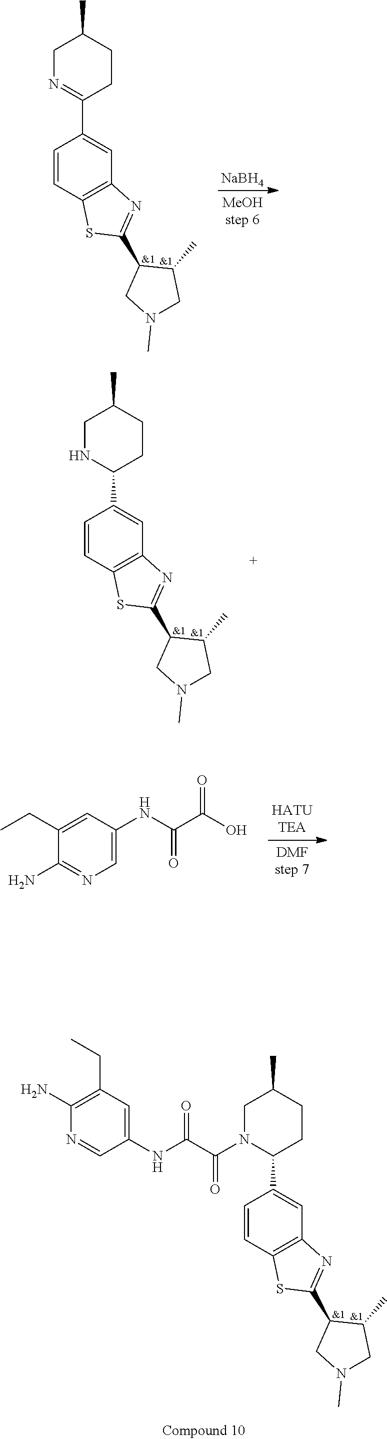

### Step 1: Synthesis of rac-(3R,4R)-1,4-dimethylpyrrolidine-3-carboxylic acid

Formaldehyde, 37% w/w aq. soln., stab. with 7-8% MeOH (3.14 g, 38.71 mmol, 2.90 mL, 37% purity) was added to the solution of rac-(3R,4R)-4-methylpyrrolidine-3-carboxylic acid (2.5 g, 19.36 mmol) in formic acid (85% in water) (18.30 g, 337.97 mmol, 15 mL, 85% purity) and resulting solution was stirred at 90° C. for 3 hr. Then, it was concentrated under reduced pressure. Residue was dissolved and co-evaporated with conc. hydrochloric acid (10 ml) in order to remove traces of paraform and formic acid, affording rac-(3R,4R)-1,4-dimethylpyrrolidine-3-carboxylic acid (3.48 g, crude, HCl).

¹H NMR (400 MHz, DMSO-d₆) δ (ppm) 1.08 (d, 3H), 2.41 (m, 1H), 2.49 (s, 3H), 2.55 (m, 2H), 3.12 (m, 3H), 7.75 (bds, 1H).

### Step 2: Synthesis of 5-bromo-2-(rac-(3S,4S)-1,4-dimethylpyrrolidin-3-yl)benzo[d]thiazole

Prepared by general procedure scheme 4.1 step 1A. Yield: 5.44 g of crude.

LCMS(ESI): [M]⁺ m/z: calcd 311.2; found 312.2; Rt=0.728 min.

### Step 3: Synthesis of 2-(rac-(3S,4S)-1,4-dimethylpyrrolidin-3-yl)-5-(4,4,5,5-tetramethyl-1,3,2-dioxaborolan-2-yl)benzo[d]thiazole

Prepared by general procedure scheme 4.1 step 2. Yield: 7.35 g of crude.

LCMS(ESI): [M]⁺ m/z: calcd 358.2; found 359.2; Rt=1.038 min.

### Step 4: Synthesis of (S)-tert-butyl 6-(2-(rac-(3S,4S)-1,4-dimethylpyrrolidin-3-yl)benzo[d]thiazol-5-yl)-3-methyl-3,4-dihydropyridine-1(2H)-carboxylate

Prepared by general procedure scheme 4.1 step 3. Yield: 9.3 g of crude.

LCMS(ESI): [M]⁺ m/z: calcd 427.2; found 428.2; Rt=1.278 min.

### Step 5: Synthesis of 2-(rac-(3S,4S)-1,4-dimethylpyrrolidin-3-yl)-5-((S)-5-methyl-3,4,5,6-tetrahydropyridin-2-yl)benzo[d]thiazole

Prepared by general procedure scheme 4.1 step 4. Yield: 5.5 g of crude.

LCMS(ESI): [M]⁺ m/z: calcd 327.2; found 328.2; Rt=0.676 min.

### Step 6: Synthesis of 2-(rac-(3S,4S)-1,4-dimethylpyrrolidin-3-yl)-5-((2R,5S)-5-methylpiperidin-2-yl)benzo[d]thiazole

Prepared by general procedure scheme 4.1 step 5. Yield: 5.1 g of crude.

LCMS(ESI): [M]⁺ m/z: calcd 329.2; found 330.2; Rt=0.687 min.

### Step 7: Synthesis of N-(6-amino-5-ethylpyridin-3-yl)-2-((2R,5S)-2-(2-(rac-(3S,4S)-1,4-dimethylpyrrolidin-3-yl)benzo[d]thiazol-5-yl)-5-methylpiperidin-1-yl)-2-oxoacetamide

Prepared by general procedure scheme 4.1 step 6A. Yield: 489 mg (39.42%).

HPLC conditions: Column: YMC Triart C18 100*20 mm, 5 microM; 0-5 min 40-80% water-MeOH+0.1% NH₄OH, flow: 30 ml/min; (loading pump 4 ml/min MeOH).

Compound 10: ¹H NMR (600 MHz, DMSO-d₆) δ (ppm) 1.09 (m, 9H), 1.35 (m, 1H), 1.71 (m, 1H), 1.87 (m, 1H), 2.12 (m, 2H), 2.28 (s, 3H), 2.39 (m, 4H), 2.88 (m, 4H), 3.28 (m, 1H), 3.86 (m, 1H), 5.64 (m, 3H), 7.37 (m, 1H), 7.47 (m, 1H), 7.87 (m, 1H), 8.03 (m, 2H), 10.55 (m, 1H).

LCMS(ESI): [M]⁺ m/z: calcd 520.2; found 521.2; Rt=2.232 min.

### Example 53. Compound 16 and Compound 4 N-(6-amino-5-ethylpyridin-3-yl)-2-((2R,5S)-2-(2-(1,5-dimethylpyrrolidin-3-yl)benzo[d]thiazol-5-yl)-5-methylpiperidin-1-yl)-2-oxoacetamide

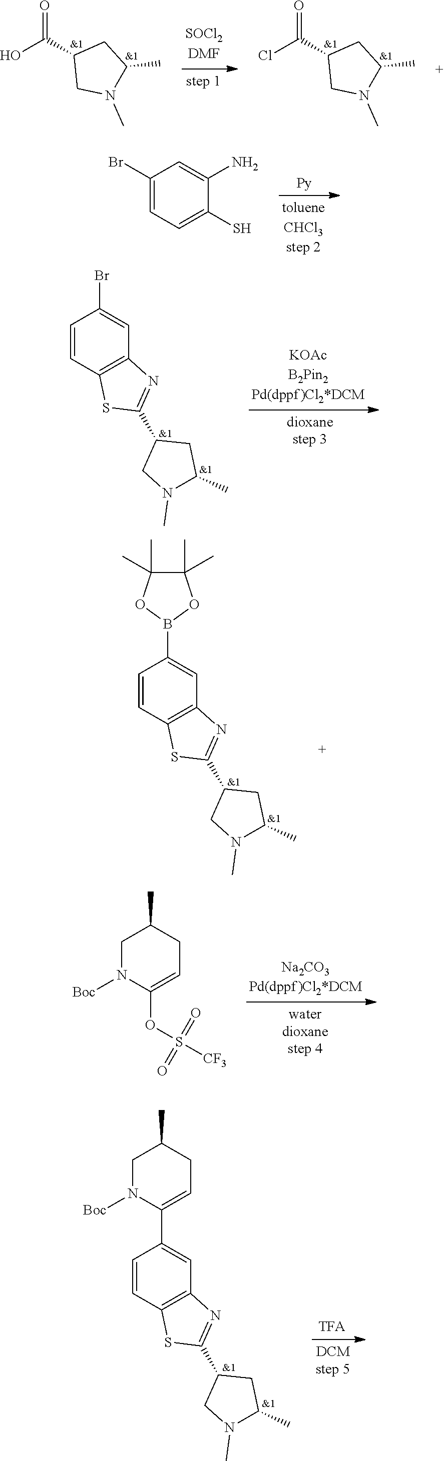

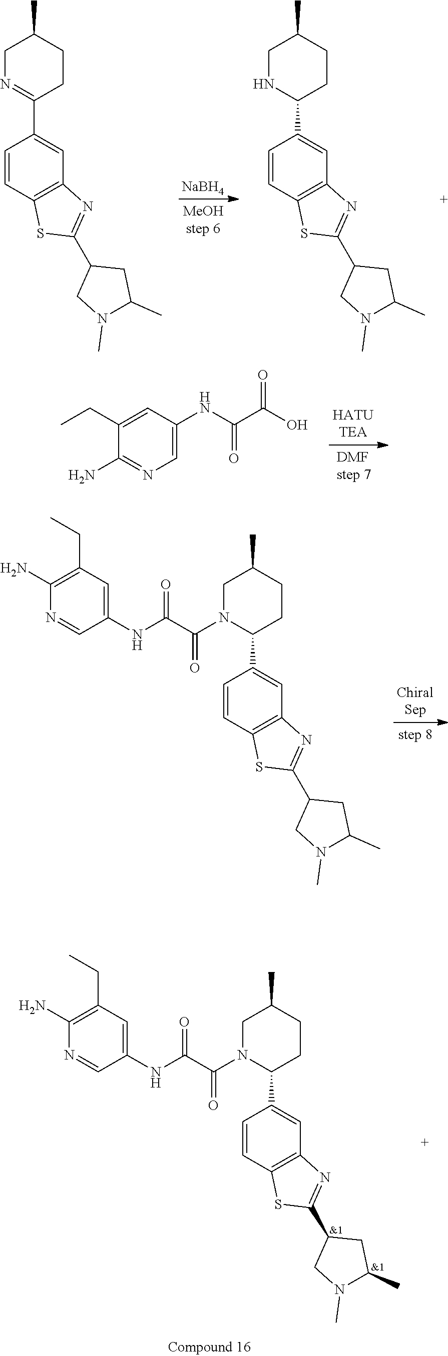 [SMILES: CC1CC(c2nc3cc(C4=NC[C@@H](C)CC4)ccc3s2)CN1C ; "NaBH4" ; CC1CC(c2nc3cc([C@H]4CC[C@H](C)CN4)ccc3s2)CN1C ; CCc1cc(NC(=O)C(=O)O)cnc1N ; CCc1cc(NC(=O)C(=O)N2C[C@@H](C)CC[C@@H]2c2ccc3sc(C4CC(C)N(C)C4)nc3c2)cnc1N ; CCc1cc(NC(=O)C(=O)N2C[C@@H](C)CC[C@@H]2c2ccc3sc([C@H]4C[C@@H](C)N(C)C4)nc3c2)cnc1N]

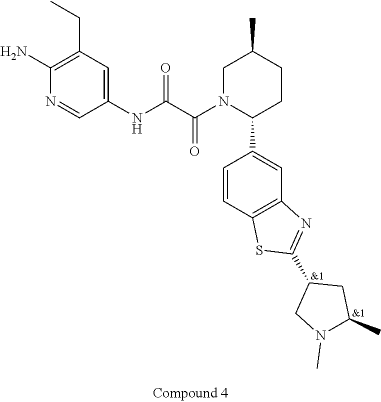 [SMILES: CCc1cc(NC(=O)C(=O)N2C[C@@H](C)CC[C@@H]2c2ccc3sc([C@@H]4C[C@@H](C)N(C)C4)nc3c2)cnc1N]

### Step 1: Synthesis of rac-(3R,5S)-1,5-dimethylpyrrolidine-3-carbonyl chloride

DMF (18.88 mg, 258.30 mol, 0.02 mL) was added to the suspension of rac-(3R,5S)-1,5-dimethylpyrrolidine-3-carboxylic acid (1 g, 5.57 mmol, HCl) in thionyl chloride (9.96 g, 83.72 mmol, 6 mL). Resulting mixture was stirred at 75° C. for 1 hr. After gas evolution ceased and solution becomes homogenous, volatiles were removed under reduced pressure, leaving rac-(3R,5S)-1,5-dimethylpyrrolidine-3-carbonyl chloride (1.1 g, 5.55 mmol, 99.76% yield, HCl).

¹H NMR (400 MHz, CDCl₃) δ (ppm) 2.41 (m, 1H), 2.75 (d, 3H), 2.89 (s, 3H), 3.50 (m, 2H), 3.99 (m, 2H), 4.21 (m, 1H).

### Step 2: Synthesis of rac-5-bromo-2-((3R,5S)-1,5-dimethylpyrrolidin-3-yl)benzo[d]thiazole

rac-(3R,5S)-1,5-Dimethylpyrrolidine-3-carbonyl chloride (1.1 g, 5.55 mmol, HCl) solution in chloroform (10 mL) was added dropwise to the solution of 2-amino-4-bromo-benzenethiol (1.13 g, 5.55 mmol) in toluene (20 mL) and pyridine (4.89 g, 61.82 mmol, 5 mL) under argon. Resulting mixture was stirred at 25° C. for 16 hr. Then, it was partitioned between 15% aq. K₂CO₃ solution (30 ml) and ethyl acetate (30 ml). Organic layer was separated, dried over Na₂SO₄ and concentrated under reduced pressure. Residue was purified by HPLC (40-40-90% 0-1-6 min H₂O/MeOH/0.1% NH₄OH, flow: 30 ml/min; column: YMC Triart C18 100×20 mm, 5 um), affording 5-bromo-2-[rac-(3R,5S)-1,5-dimethylpyrrolidin-3-yl]-1,3-benzothiazole (722 mg, 2.32 mmol, 41.77% yield).

LCMS(ESI): [M]⁺ m/z: calcd 311.2; found 312.2; Rt=1.531 min.

### Step 3: Synthesis of rac-2-((3R,5S)-1,5-dimethylpyrrolidin-3-yl)-5-(4,4,5,5-tetramethyl-1,3,2-dioxaborolan-2-yl)benzo[d]thiazole

Prepared by general procedure scheme 4.1 step 2. Yield: 1.02 g of crude.

LCMS(ESI): [M]⁺ m/z: calcd 358.2; found 359.2; Rt=1.148 min.

### Step 4: Synthesis of (S)-tert-butyl 6-(2-(rac-(3R,5S)-1,5-dimethylpyrrolidin-3-yl)benzo[d]thiazol-5-yl)-3-methyl-3,4-dihydropyridine-1(2H)-carboxylate

Prepared by general procedure scheme 4.1 step 3. Yield: 1.35 g of crude.

LCMS(ESI): [M]⁺ m/z: calcd 427.2; found 428.2; Rt=1.230 min.

### Step 5: Synthesis of 2-(1,5-dimethylpyrrolidin-3-yl)-5-((S)-5-methyl-3,4,5,6-tetrahydropyridin-2-yl)benzo[d]thiazole

Prepared by general procedure scheme 4.1 step 4. Yield: 0.8 g of crude.

LCMS(ESI): [M]⁺ m/z: calcd 327.2; found 328.2; Rt=0.610 min.

### Step 6: Synthesis of 2-(1,5-dimethylpyrrolidin-3-yl)-5-((2R,5S)-5-methylpiperidin-2-yl)benzo[d]thiazole

Prepared by general procedure scheme 4.1 step 5. Yield: 0.73 g of crude.

LCMS(ESI): [M]⁺ m/z: calcd 329.2; found 330.2; Rt=0.683 min.

### Step 7: Synthesis of N-(6-amino-5-ethylpyridin-3-yl)-2-((2R,5S)-2-(2-(1,5-dimethylpyrrolidin-3-yl)benzo[d]thiazol-5-yl)-5-methylpiperidin-1-yl)-2-oxoacetamide

Prepared by general procedure scheme 4.1 step 6A. Yield: 385 mg (33.37%).

HPLC conditions: Column: YMC Triart C18 100*20 mm, 5 microM; 0-5 min 40-80% water-MeOH+0.1% NH₄OH, flow: 30 ml/min; (loading pump 4 ml/min MeOH).

¹H NMR (600 MHz, DMSO-d₆) δ (ppm)

LCMS(ESI): [M]⁺ m/z: calcd 520.2; found 521.2; Rt=2.176 min.

### Step 8: Chiral Separation (Compound 16 and Compound 4)

Racemic N-(6-amino-5-ethyl-3-pyridyl)-2-oxo-2-[(2R,5S)-2-[2-(1,5-dimethylpyrrolidin-3-yl)-1,3-benzothiazol-5-yl]-5-methyl-1-piperidyl]acetamide (300 mg, 576.16 μmol) was chiral separated (Column: Chiralcel OJ-I (250*20 mm, 5 mkm); Mobile phase: Hexane-IPA-MeOH, 60-20-20. Flow Rate: 12 mL/min) to obtain N-(6-amino-5-ethyl-3-pyridyl)-2-oxo-2-[(2R,5S)-5-methyl-2-[2-[rac-(3S,5R)-1,5-dimethylpyrrolidin-3-yl]-1,3-benzothiazol-5-yl]-1-piperidyl]acetamide (84 mg, 161.33 μmol, 56.00% yield) (RT=15.68 min) and N-(6-amino-5-ethyl-3-pyridyl)-2-oxo-2-[(2R,5S)-5-methyl-2-[2-[rac-(3R,5R)-1,5-dimethylpyrrolidin-3-yl]-1,3-benzothiazol-5-yl]-1-piperidyl]acetamide (82 mg, 157.48 mol, 54.67% yield) (RT=20.36 min).

Rel Time for Compound 16 in analytical conditions (column: IC, MeOH-IPA, 50-50, 0.6 ml/min as mobile phase) 10.7 min and for Compound 4 14.45 min.

### Compound 16: Retention Time: 10.7 Min

¹H NMR (600 MHz, DMSO-d₆) δ (ppm) 1.07 (m, 9H), 1.35 (m, 1H), 1.70 (m, 1H), 1.92 (m, 2H), 2.13 (m, 1H), 2.24 (s, 3H), 2.28 (m, 2H), 2.40 (m, 4H), 2.78 (m, 1H), 3.43 (m, 1H), 3.91 (m, 2H), 5.64 (m, 3H), 7.41 (m, 2H), 7.87 (m, 1H), 8.04 (m, 2H), 10.55 (m, 1H).

LCMS(ESI): [M]⁺ m/z: calcd 520.2; found 521.2; Rt=1.861 min.

### Compound 4: Retention Time: 14.45 Min

¹H NMR (600 MHz, DMSO-d₆) δ (ppm) 1.09 (m, 9H), 1.35 (m, 1H), 1.68 (m, 2H), 1.87 (m, 1H), 2.13 (m, 1H), 2.24 (s, 4H), 2.37 (m, 3H), 2.57 (m, 2H), 2.78 (m, 2H), 3.87 (m, 2H), 5.64 (m, 3H), 7.36 (m, 1H), 7.47 (m, 1H), 7.83 (m, 1H), 8.02 (m, 2H), 10.55 (m, 1H).

LCMS(ESI): [M]⁺ m/z: calcd 520.2; found 521.2; Rt=1.842 min.

### Example 54. Compound 120 5-(2-((2R,5S)-2-(2-(2-(dimethylamino)-2-methylpropyl)benzo[d]thiazol-5-yl)-5-methylpiperidin-1-yl)-2-oxoacetamido)-2-methoxynicotinamide

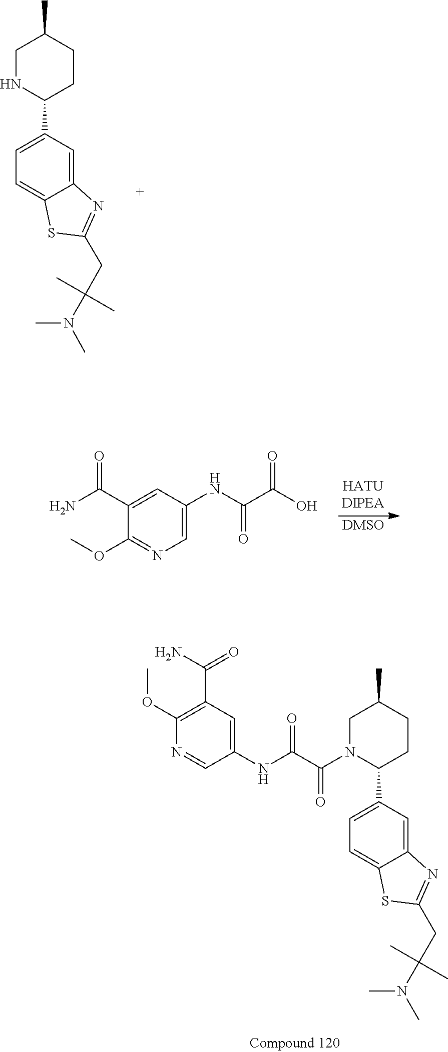

Synthesis of N,N,2-trimethyl-1-(5-((2R,5S)-5-methylpiperidin-2-yl)benzo[d]thiazol-2-yl)propan-2-amine is given by 3HHH.

Prepared by general procedure scheme 4.1 step 6A. Yield: 10 mg (7.5%).

HPLC conditions: Column: SunFire C18 100*19 mm, 5 microM; 2-10 min 50-75% water-MeOH+NH₃; (loading pump 4 ml/min MeOH).

Compound 120: ¹H NMR (600 MHz, DMSO-d₆) δ (ppm) 1.05 (m, 9H), 1.37 (m, 1H), 1.72 (m, 1H), 1.89 (m, 1H), 2.14 (m, 1H), 2.28 (m, 6H), 3.21 (m, 3H), 3.94 (m, 5H), 5.49 (m, 1H), 7.35 (dd, 1H), 7.73 (m, 2H), 7.85 (m, 1H), 8.00 (m, 1H), 8.49 (m, 2H), 11.07 (m, 1H).

LCMS(ESI): [M]⁺ m/z: calcd 552.2; found 553.2; Rt=2.478 min.

### Example 55. Compound 94 5-(2-((2R,5S)-2-(2-(1,5-dimethylpiperidin-3-yl)benzo[d]thiazol-5-yl)-5-methylpiperidin-1-yl)-2-oxoacetamido)-2-methoxynicotinamide

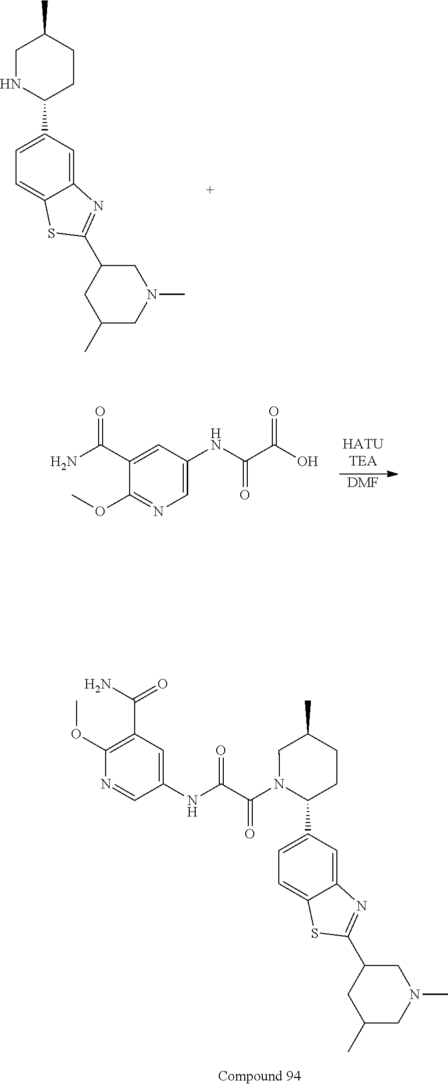

Synthesis of 2-(1,5-dimethylpiperidin-3-yl)-5-((2R,5S)-5-methylpiperidin-2-yl)benzo[d]thiazole is given by Scheme 4.1, steps 1-6.

Prepared by general procedure scheme 4.1 step 6A. Yield: 127 mg (30.9%).

HPLC conditions: Column: XBridge BEH C18 100*19 mm, 5 microM; 0-6 min 50-90% water-MeOH+0.1% NH₄OH; (loading pump 4 ml/min MeOH).

Compound 94: LCMS(ESI): [M]⁺ m/z: calcd 564.2; found 565.2; Rt=1.038 min.

### Example 56. Compound 70 N-(6-amino-5-ethylpyridin-3-yl)-2-((2R,5S)-2-(2-(1,4-dimethylpiperazin-2-yl)benzo[d]thiazol-5-yl)-5-methylpiperidin-1-yl)-2-oxoacetamide

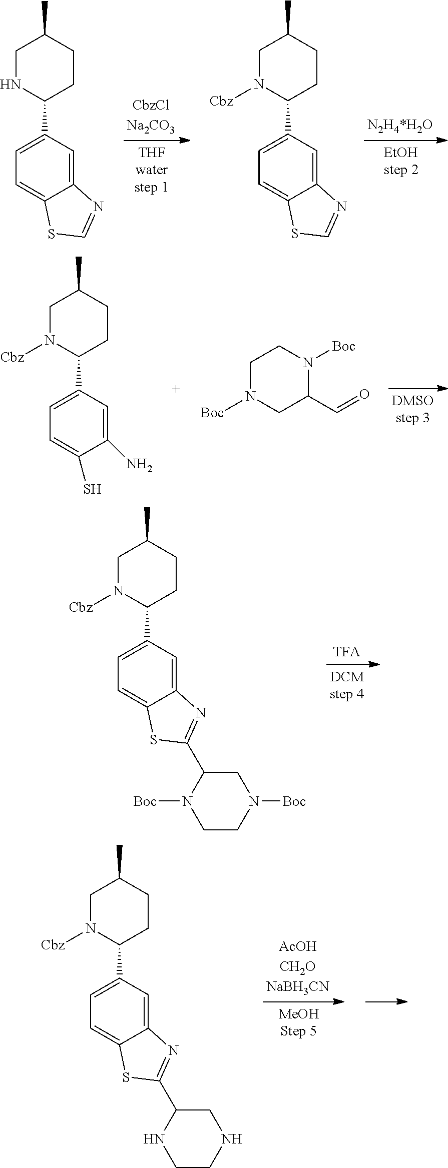

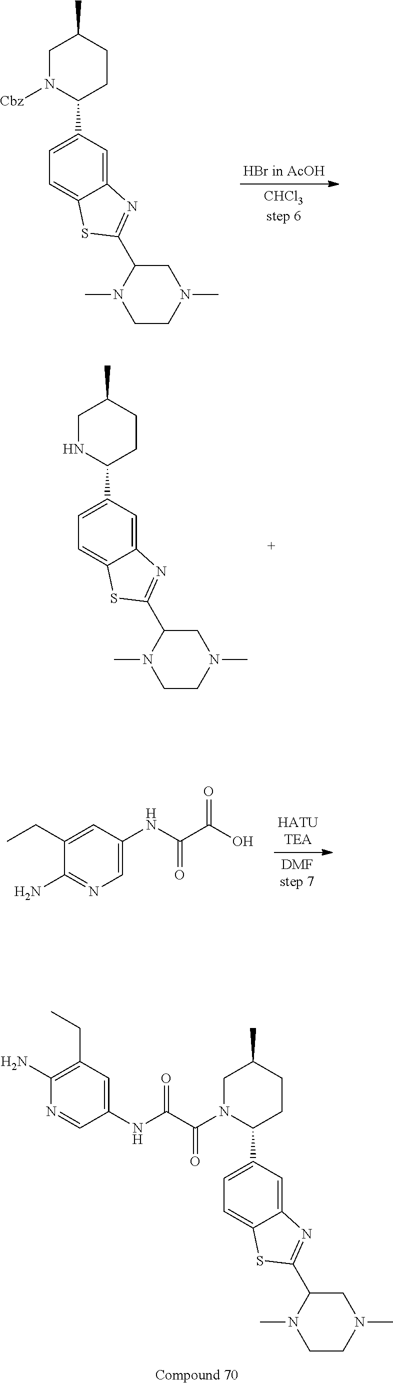 [SMILES: C[C@H]1CC[C@H](c2ccc3sc(C4CN(C)CCN4C)nc3c2)N(C(=O)OCc2ccccc2)C1 ; C[C@H]1CC[C@H](c2ccc3sc(C4CN(C)CCN4C)nc3c2)NC1 ; CCc1cc(NC(=O)C(=O)O)cnc1N ; CCc1cc(NC(=O)C(=O)N2C[C@@H](C)CC[C@@H]2c2ccc3sc(C4CN(C)CCN4C)nc3c2)cnc1N]

### Step 1: Synthesis of (2R,5S)-benzyl 2-(benzo[d]thiazol-5-yl)-5-methylpiperidine-1-carboxylate

Benzyl chloroformate (1.55 g, 8.19 mmol, 90% purity) was added dropwise to the solution of sodium carbonate (2.48 g, 23.41 mmol, 979.84 μL) and 5-[(2R,5S)-5-methyl-2-piperidyl]-1,3-benzothiazole (3 g, 7.80 mmol) in THF (40 mL) and water (20 mL). Resulting mixture was stirred at 25° C. for 3 hr. Then, it was partitioned between ethyl acetate (50 ml) and water (60 ml). Organic layer was separated, dried over Na₂SO₄ and concentrated under reduced pressure, leaving benzyl (2R,5S)-2-(1,3-benzothiazol-5-yl)-5-methyl-piperidine-1-carboxylate (2.9 g, crude).

LCMS(ESI): [M]⁺ m/z: calcd 366.2; found 367.2; Rt=1.495 min.

### Step 2: Synthesis of (2R,5S)-benzyl 2-(3-amino-4-mercaptophenyl)-5-methylpiperidine-1-carboxylate

Hydrazine hydrate solution 55% in water (35% hydrazine) (11.32 g, 79.13 mmol, 11.02 mL, 35% purity) was added to the solution of benzyl (2R,5S)-2-(1,3-benzothiazol-5-yl)-5-methyl-piperidine-1-carboxylate (2.9 g, 7.91 mmol) in EtOH (40 mL). Resulting mixture was stirred at 78° C. for 72 hr. Then, volatiles were removed under reduced pressure and residue was diluted with water (50 ml) and extracted with ethyl acetate (2×40 ml). Combined organic layers were dried over Na₂SO₄ and concentrated under reduced pressure, leaving benzyl (2R,5S)-2-(3-amino-4-sulfanyl-phenyl)-5-methyl-piperidine-1-carboxylate (2.5 g, 7.01 mmol, 88.62% yield).

LCMS(ESI): [M]⁺ m/z: calcd 356.2; found 357.2; Rt=1.481 min.

### Step 3: Synthesis of di-tert-butyl 2-(5-((2R,5S)-1-((benzyloxy)carbonyl)-5-methylpiperidin-2-yl)benzo[d]thiazol-2-yl)piperazine-1,4-dicarboxylate

Prepared by general procedure scheme 4.1 step 1B. Yield: 1.5 g of crude.

LCMS(ESI): [M]⁺ m/z: calcd 650.2; found 651.2; Rt=1.726 min.

### Step 4: Synthesis of (2R,5S)-benzyl 5-methyl-2-(2-(piperazin-2-yl)benzo[d]thiazol-5-yl)piperidine-1-carboxylate

TFA (7.40 g, 64.90 mmol, 5 mL) was added to the solution of di-tert-butyl 2-[5-[(2R,5S)-1-benzyloxycarbonyl-5-methyl-2-piperidyl]-1,3-benzothiazol-2-yl]piperazine-1,4-dicarboxylate (1.5 g, 576.19 mol) in DCM (20 mL). Resulting mixture was stirred at 30° C. for 4 hr. Then, volatiles were removed under reduced pressure and residue was taken up in water (40 ml). Insoluble tar material was filtered off through a cotton wool plug. Clear filtrate was basified with solid K₂CO₃ to pH≥10 and extracted with DCM (3×15 ml). Combined organic layers were dried over K₂CO₃ and concentrated in vacuum, affording benzyl (2R,5S)-5-methyl-2-(2-piperazin-2-yl-1,3-benzothiazol-5-yl)piperidine-1-carboxylate (190 mg, 421.66 mol, 73.18% yield).

LCMS(ESI): [M]⁺ m/z: calcd 450.2; found 451.2; Rt=1.257 min.

### Step 5: Synthesis of (2R,5S)-benzyl 2-(2-(1,4-dimethylpiperazin-2-yl)benzo[d]thiazol-5-yl)-5-methylpiperidine-1-carboxylate

Formaldehyde, 37% w/w aq. soln., stab. with 7-8% MeOH (136.87 mg, 1.69 mmol, 126.38 μL, 37% purity) and acetic acid (101.29 mg, 1.69 mmol, 96.56 μL) were added to the solution of benzyl (2R,5S)-5-methyl-2-(2-piperazin-2-yl-1,3-benzothiazol-5-yl)piperidine-1-carboxylate (190 mg, 421.66 mol) in MeOH (10 mL). Then, sodium cyan borohydride (105.99 mg, 1.69 mmol) was added thereto. Resulting mixture was stirred at 30° C. for 18 hr. After that, volatiles were removed under reduced pressure and residue was partitioned between 15% aq. K₂CO₃ solution (10 ml) and DCM (20 ml). Organic layer was separated, dried over solid K₂CO₃ and concentrated under reduced pressure, leaving 220 mg of crude product, which was purified by HPLC (50-50-95% 0-1-6 min H₂O/MeOH/0.1% NH₄OH, flow: 30 ml/min; column: XBridge BEH C18 5 μm 130 A), affording benzyl (2R,5S)-2-[2-(1,4-dimethylpiperazin-2-yl)-1,3-benzothiazol-5-yl]-5-methyl-piperidine-1-carboxylate (77 mg, 160.87 μmol, 38.15% yield).

LCMS(ESI): [M]⁺ m/z: calcd 478.2; found 479.2; Rt=3.463 min.

### Step 6: Synthesis of 2-(1,4-dimethylpiperazin-2-yl)-5-((2R,5S)-5-methylpiperidin-2-yl)benzo[d]thiazole

Hydrogen bromide 30% in AcOH (1.35 g, 5.02 mmol, 1 mL, 30% purity) was added to the solution of benzyl (2R,5S)-2-[2-(1,4-dimethylpiperazin-2-yl)-1,3-benzothiazol-5-yl]-5-methyl-piperidine-1-carboxylate (55 mg, 114.91 mol) in CHCl₃ (5 mL). Resulting mixture was stirred at 30° C. for 5 hr. Then, volatiles were removed under reduced pressure and residue was washed with hexane, leaving 2-(1,4-dimethylpiperazin-2-yl)-5-[(2R,5S)-5-methyl-2-piperidyl]-1,3-benzothiazole (67 mg, 114.09 mol, 99.29% yield, 3HBr).

LCMS(ESI): [M]⁺ m/z: calcd 344.2; found 345.2; Rt=0.736 min.

### Step 7: Synthesis of N-(6-amino-5-ethylpyridin-3-yl)-2-((2R,5S)-2-(2-(1,4-dimethylpiperazin-2-yl)benzo[d]thiazol-5-yl)-5-methylpiperidin-1-yl)-2-oxoacetamide (Compound 70)

Prepared by general procedure scheme 4.1 step 6A. Yield: 7.3 mg (11.94%).

HPLC conditions: Column: YMC Triart C18 100*20 mm, 5 microM; 0-1.5-6 min 70% water-MeOH+0.1% NH₄OH, flow: 30 ml/min; (loading pump 4 ml/min MeOH).

Compound 70: ¹H NMR (600 MHz, DMSO-d₆) δ (ppm) 1.01-1.14 (m, 6H), 1.31-1.41 (m, 1H), 1.67-1.76 (m, 1H), 1.81-1.93 (m, 1H), 2.04-2.36 (m, 11H), 2.39-2.42 (m, 1H), 2.66-3.11 (m, 4H), 3.42-4.07 (m, 2H), 5.26-5.73 (m, 3H), 7.36-7.52 (m, 2H), 7.87-7.95 (m, 1H), 7.97-8.12 (m, 2H), 8.49-8.99 (m, 1H), 10.28-10.78 (m, 1H).

LCMS(ESI): [M]⁺ m/z: calcd 535.2; found 536.2; Rt=2.182 min.

### Example 57. Compound 22 5-(2-((2R,5S)-2-(2-((1,4-dimethylpiperidin-4-yl)methyl)benzo[d]thiazol-5-yl)-5-methylpiperidin-1-yl)-2-oxoacetamido)-2-methoxynicotinamide

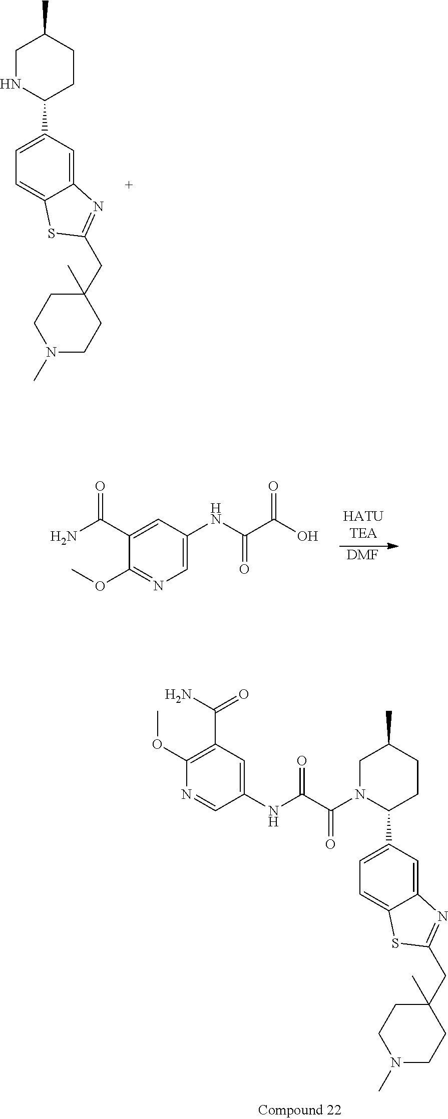

The synthesis of 2-((1,4-dimethylpiperidin-4-yl)methyl)-5-((2R,5S)-5-methylpiperidin-2-yl)benzo[d]thiazole is given by 3SSS.

Prepared by general procedure scheme 4.1 step 6A. Yield: 84.8 mg (52.39%).

HPLC conditions: Column: SunFire C18 100*19 mm, 5 microM; 0-5 min 25-50% water-MeCN+NH₃; (loading pump 4 ml/min MeCN).

Compound 22: ¹H NMR (600 MHz, DMSO-d₆) δ (ppm) 0.73-1.00 (m, 3H), 1.00-1.11 (m, 3H), 1.29-1.44 (m, 3H), 1.56-1.64 (m, 2H), 1.68-1.76 (m, 1H), 1.82-1.96 (m, 1H), 1.97-2.12 (m, 1H), 2.14 (s, 3H), 2.16-2.24 (m, 2H), 2.24-2.36 (m, 1H), 2.39-2.46 (m, 2H), 2.82-2.87 (m, 0.3H), 2.99-3.06 (m, 2H), 3.23-3.29 (m, 0.7H), 3.48-3.53 (m, 0.7H), 3.88-3.99 (m, 3H), 4.02-4.06 (m, 0.3H), 5.20-5.81 (m, 1H), 7.34-7.45 (m, 1H), 7.65-7.79 (m, 2H), 7.85-7.94 (m, 1H), 7.99-8.09 (m, 1H), 8.38-8.62 (m, 2H), 10.50-11.56 (m, 1H).

LCMS(ESI): [M]⁺ m/z: calcd 578.2; found 579.2; Rt=1.097 min.

### Example 58. Compound 125 N-(6-amino-5-ethylpyridin-3-yl)-2-((2R,5S)-2-(2-(1,4-dimethyl-6-oxopiperazin-2-yl)benzo[d]thiazol-5-yl)-5-methylpiperidin-1-yl)-2-oxoacetamide

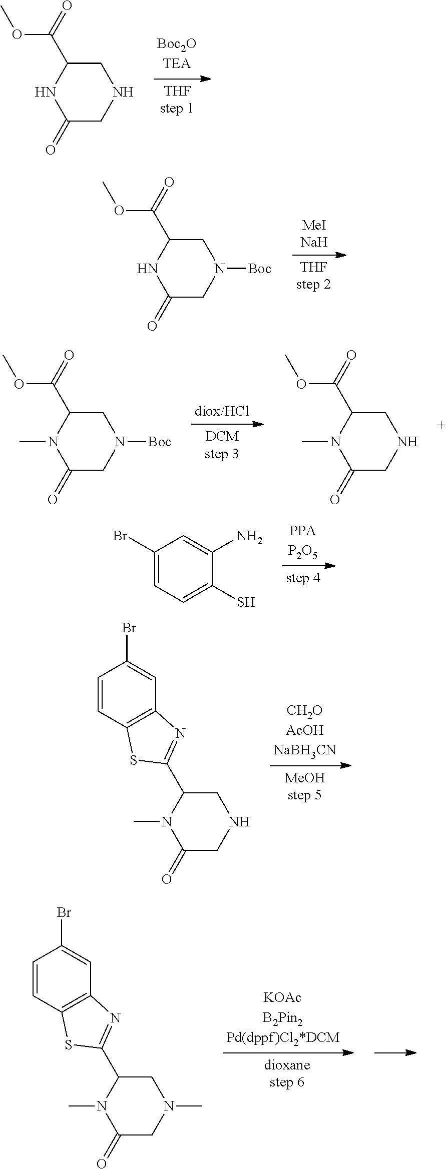

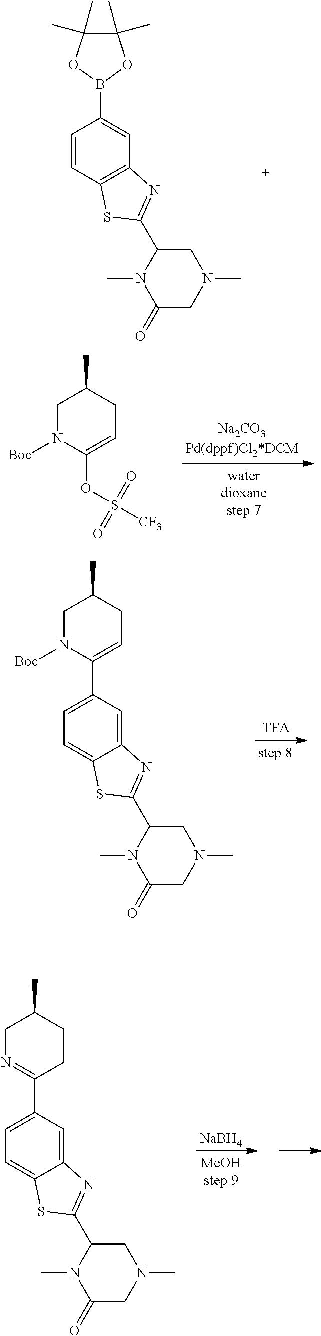

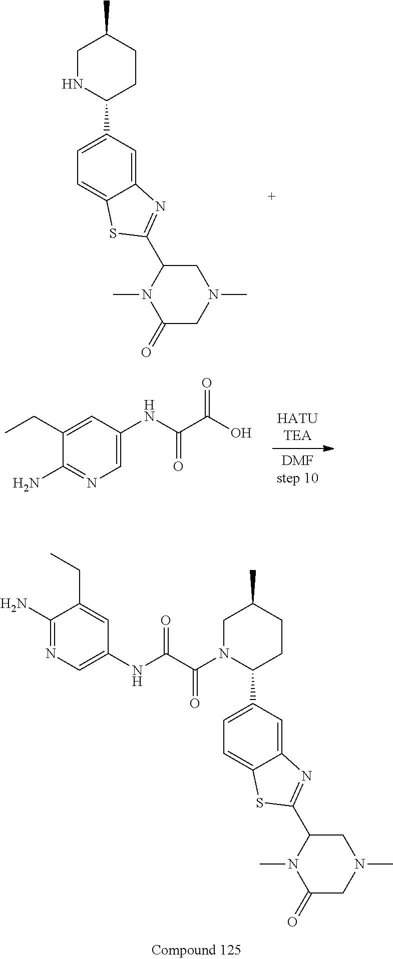 [SMILES: C[C@H]1CC[C@H](c2ccc3sc(C4CN(C)CC(=O)N4C)nc3c2)NC1 ; CCc1cc(NC(=O)C(=O)O)cnc1N ; CCc1cc(NC(=O)C(=O)N2C[C@@H](C)CC[C@@H]2c2ccc3sc(C4CN(C)CC(=O)N4C)nc3c2)cnc1N]

### Step 1: Synthesis of 1-tert-butyl 3-methyl 5-oxopiperazine-1,3-dicarboxylate

tert-Butoxycarbonyl tert-butyl carbonate (6.06 g, 27.75 mmol, 6.37 mL) was added dropwise to a stirred suspension of methyl 6-oxopiperazine-2-carboxylate (5.4 g, 27.75 mmol, HCl) and TEA (2.81 g, 27.75 mmol, 3.87 mL) in THF (101.14 mL) at 20° C. The resulting mixture was stirred at 20° C. for 12 hr, then it was filtered off. The filtrate was evaporated in vacuum to give 1-tert-butyl 3-methyl 5-oxopiperazine-1,3-dicarboxylate (7 g, 27.10 mmol, 97.68% yield) as white solid.

LCMS(ESI): [M]⁺ m/z: calcd 258.2; found 259.2; Rt=0.938 min.

### Step 2: Synthesis of 1-tert-butyl 3-methyl 4-methyl-5-oxopiperazine-1,3-dicarboxylate

To a solution of 1-tert-butyl 3-methyl 5-oxopiperazine-1,3-dicarboxylate (7 g, 27.10 mmol) in THF (70 mL) sodium hydride (in oil dispersion) 60% dispersion in mineral oil (1.08 g, 27.10 mmol, 60% purity) was added portion wise at rt. The resulting mixture was stirred until hydrogen evolution ceased, then iodomethane (4.04 g, 28.46 mmol, 1.77 mL) was added in one portion. The resulting mixture was stirred at rt. for 12 hr and then evaporated in vacuum. The residue was diluted with water (75 mL) and extracted with DCM (2*80 mL). The combined organic extracts were dried over sodium sulphate and evaporated in vacuum to afford 1-tert-butyl 3-methyl 4-methyl-5-oxopiperazine-1,3-dicarboxylate (8 g, crude).

¹H NMR (500 MHz, DMSO-d₆) δ (ppm) 1.35 (s, 9H), 2.79 (s, 3H), 3.59 (s, 3H), 3.69 (m, 2H), 4.18 (m, 2H), 4.31 (m, 1H).

### Step 3: Synthesis of methyl 1-methyl-6-oxopiperazine-2-carboxylate

Hydrogen chloride solution 4.0M in dioxane (107.12 g, 293.80 mmol, 102.02 mL, 10% purity) was carefully added at rt. to a solution of 1-tert-butyl 3-methyl 4-methyl-5-oxopiperazine-1,3-dicarboxylate (8 g, 29.38 mmol) in DCM. The reaction mixture was then stirred for 12 hr at rt. Then it was evaporated in vacuum to give methyl 1-methyl-6-oxo-piperazine-2-carboxylate (6 g, 28.76 mmol, 97.88% yield, HCl).

LCMS(ESI): [M]⁺ m/z: calcd 172.2; found 173.2; Rt=0.193 min.

### Step 4: Synthesis of 6-(5-bromobenzo[d]thiazol-2-yl)-1-methylpiperazin-2-one

Phosphoric acid (10.52 g, 91.26 mmol, 6.19 mL, 85% purity) and phosphorus pentoxide (13.14 g, 92.59 mmol) were mixed together. The reaction suspension was stirred at rt for 10 min, then 2-amino-4-bromo-benzenethiol (4.45 g, 21.79 mmol) followed by methyl 1-methyl-6-oxo-piperazine-2-carboxylate (5 g, 23.96 mmol, HCl) were added. The solution was stirred at 110° C. for 18 hr then it was triturated with water, basified (NaOH, 10% aq.) to pH8, extracted with EtOAc (2×50 mL), dried and evaporated in vacuum. The residue was purified by HPLC (15-15-45% 0-1-6 min H₂O/MeCN/0.1% NH₄OH) to afford product 6-(5-bromo-1,3-benzothiazol-2-yl)-1-methyl-piperazin-2-one (61 mg, 187.00 μmol, 8.58e-1% yield).

LCMS(ESI): [M]⁺ m/z: calcd 326.2; found 327.2; Rt=0.847 min.

### Step 5: Synthesis of 6-(5-bromobenzo[d]thiazol-2-yl)-1,4-dimethylpiperazin-2-one

A mixture of 6-(5-bromo-1,3-benzothiazol-2-yl)-1-methyl-piperazin-2-one (63 mg, 193.13 mol), formaldehyde (21.94 mg, 270.38 mol, 20.26 μL) and acetic acid (23.19 mg, 386.25 μmol, 22.11 μL) in MeOH (2 mL) was stirred at rt for 2 hr. To the mixture was then added sodium cyan borohydride (24.27 mg, 386.25 mol) in one portion. The mixture was stirred at 20° C. for 18 hr, then concentrated in vacuum. The residue was basified to pH9 (K₂CO₃ 10% aq.) and extracted with DCM (2*5 mL). The combined organic extracts were dried over sodium sulphate and evaporated in vacuum to afford 6-(5-bromo-1,3-benzothiazol-2-yl)-1,4-dimethyl-piperazin-2-one (100 mg, crude).

LCMS(ESI): [M]⁺ m/z: calcd 340.2; found 341.2; Rt=1.166 min.

### Step 6: Synthesis of 1,4-dimethyl-6-(5-(4,4,5,5-tetramethyl-1,3,2-dioxaborolan-2-yl)benzo[d]thiazol-2-yl)piperazin-2-one

Prepared by general procedure scheme 4.1 step 2. Yield: 113 mg of crude.

LCMS(ESI): [M]⁺ m/z: calcd 387.2; found 388.2; Rt=1.338 min.

### Step 7: Synthesis of (3S)-tert-butyl 6-(2-(1,4-dimethyl-6-oxopiperazin-2-yl)benzo[d]thiazol-5-yl)-3-methyl-3,4-dihydropyridine-1(2H)-carboxylate

Prepared by general procedure scheme 4.1 step 3. Yield: 200 mg of crude.

LCMS(ESI): [M]⁺ m/z: calcd 456.2; found 457.2; Rt=1.484 min.

### Step 8: Synthesis of 1,4-dimethyl-6-(5-((S)-5-methyl-3,4,5,6-tetrahydropyridin-2-yl)benzo[d]thiazol-2-yl)piperazin-2-one

Prepared by general procedure scheme 4.1 step 4. Yield: 200 mg of crude.

LCMS(ESI): [M]⁺ m/z: calcd 356.2; found 357.2; Rt=0.786 min.

### Step 9: Synthesis of 1,4-dimethyl-6-(5-((2R,5S)-5-methylpiperidin-2-yl)benzo[d]thiazol-2-yl)piperazin-2-one

Prepared by general procedure scheme 4.1 step 5. Yield: 200 mg of crude.

LCMS(ESI): [M]⁺ m/z: calcd 358.2; found 359.2; Rt=0.682 min.

### Step 10: Synthesis of N-(6-amino-5-ethylpyridin-3-yl)-2-((2R,5S)-2-(2-(1,4-dimethyl-6-oxopiperazin-2-yl)benzo[d]thiazol-5-yl)-5-methylpiperidin-1-yl)-2-oxoacetamide (Compound 125)

Prepared by general procedure scheme 4.1 step 6A. Yield: 16 mg (10.44%).

HPLC conditions: Column: XBridge BEH C18 100*19 mm, 5 microM; 0-1-6 min 25-25-40% water-MeCN+0.1% NH₄OH, flow: 30 ml/min; (loading pump 4 ml/min MeCN).

Compound 125: ¹H NMR (600 MHz, DMSO-d₆) δ (ppm) 1.00-1.05 (m, 3H), 1.06-1.14 (m, 3H), 1.29-1.39 (m, 1H), 1.66-2.03 (m, 3H), 2.04-2.24 (m, 5H), 2.28-2.36 (m, 2H), 2.82-2.88 (m, 5H), 3.05-3.10 (m, 1H), 3.36-4.09 (m, 2H), 4.95 (s, 1H), 5.27-5.73 (m, 3H), 7.38-7.54 (m, 2H), 7.88-7.96 (m, 1H), 7.97-8.11 (m, 2H), 10.50-10.60 (m, 1H).

LCMS(ESI): [M]⁺ m/z: calcd 549.2; found 550.2; Rt=2.371 min.

### Example 59. Compound 52 2-methoxy-5-(2-((2R,5S)-5-methyl-2-(2-(piperidin-4-yl)benzo[d]thiazol-5-yl)piperidin-1-yl)-2-oxoacetamido)nicotinamide

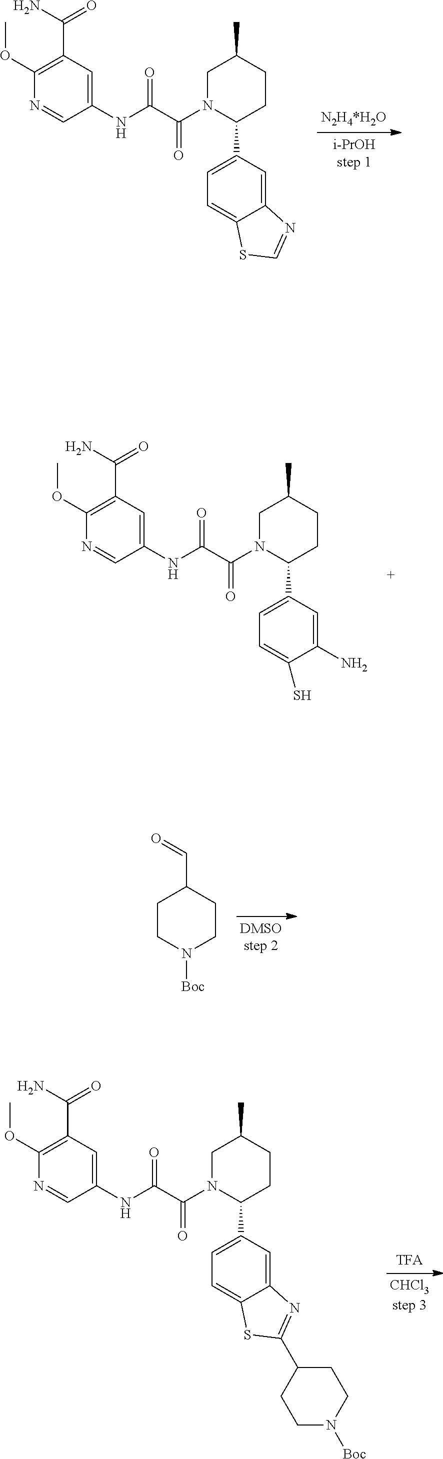 [SMILES: COc1ncc(NC(=O)C(=O)N2C[C@@H](C)CC[C@@H]2c2ccc3scnc3c2)cc1C(N)=O ; COc1ncc(NC(=O)C(=O)N2C[C@@H](C)CC[C@@H]2c2ccc(S)c(N)c2)cc1C(N)=O ; CC(C)(C)OC(=O)N1CCC(C=O)CC1 ; COc1ncc(NC(=O)C(=O)N2C[C@@H](C)CC[C@@H]2c2ccc3sc(C4CCN(C(=O)OC(C)(C)C)CC4)nc3c2)cc1C(N)=O]

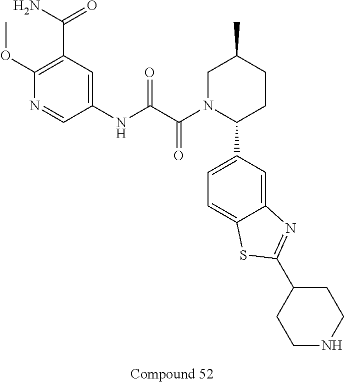 [SMILES: COc1ncc(NC(=O)C(=O)N2C[C@@H](C)CC[C@@H]2c2ccc3sc(C4CCNCC4)nc3c2)cc1C(N)=O]

HATU (4.05 g, 10.65 mmol) was added portion wise at rt to a suspension of 2-[(5-carbamoyl-6-methoxy-3-pyridyl)amino]-2-oxo-acetic acid (2.83 g, 10.65 mmol), 5-[(2R,5S)-5-methyl-2-piperidyl]-1,3-benzothiazole (2.25 g, 9.68 mmol) and TEA (5.88 g, 58.10 mmol, 8.10 mL) in DMF (35 mL). The clear solution was stirred at 20° C. for 32 hr and the solvents were evaporated in vacuum. The residue was dissolved in EtOAc (300 mL), washed with water (5×100 mL), evaporated in vacuum and purified by silica gel flash chromatography eluting with a 0 to 100 percent MeCN-chloroform gradient to give 3.4 g of product with cis impurity. The individual trans-isomer was separated by chiral HPLC (column: IB (250*30, 5 mkm), Hexane-IPA-MeOH, 70-15-15, 12 ml/min as mobile phase) to give 1.15 g of product with solvents. It was dissolved in 20 mL of dry methanol, evaporated in vacuum and dried in vacuum for 24 hr at 45° C. to 5-[[2-[(2R,5S)-2-(1,3-benzothiazol-5-yl)-5-methyl-1-piperidyl]-2-oxoacetyl]amino]-2-methoxy-pyridine-3-carboxamide (0.95 g, 2.09 mmol, 21.63% yield). [α]21D=+159.6° (c=0.1 g/100 mL EtOH).

¹H NMR (600 MHz, DMSO-d₆) δ (ppm) 0.99-1.10 (m, 3H), 1.31-1.42 (m, 1H), 1.64-1.79 (m, 1H), 1.83-1.98 (m, 1H), 2.09-2.24 (m, 1H), 2.26-2.35 (m, 1H), 2.78-3.24 (m, 1H), 3.47-3.56 (m, 0.7H), 3.88-3.99 (m, 3H), 4.02-4.07 (m, 0.3H), 5.26-5.80 (m, 1H), 7.38-7.57 (m, 1H), 7.66-7.78 (m, 2H), 7.98-8.04 (m, 1H), 8.13-8.20 (m, 1H), 8.38-8.59 (m, 2H), 9.25-9.53 (m, 1H), 10.97-11.17 (m, 1H).

LCMS(ESI): [M]⁺ m/z: calcd 453.2; found 454.2; Rt=2.672 min.

### Step 1: Synthesis of 5-(2-((2R,5S)-2-(3-amino-4-mercaptophenyl)-5-methylpiperidin-1-yl)-2-oxoacetamido)-2-methoxynicotinamide

2-Methoxy-5-[[2-oxo-2-[(2R,5S)-2-(1,3-benzothiazol-5-yl)-5-methyl-1-piperidyl]acetyl]amino]pyridine-3-carboxamide (0.4 g, 882.00 mol) and hydrazine monohydrate (205.40 mg, 4.10 mmol, 0.2 mL) were mixed together in i-PrOH (10 mL) under argon atmosphere and the resulting solution was refluxed overnight. Upon completion, the reaction mixture was evaporated, the crude product was quenched with water (10 mL). The aqueous phase was extracted with CHCl₃ (2*10 mL). The combined organic phase was dried over Na₂SO₄ and concentrated under reduced pressure. The desired product 2-methoxy-5-[[2-oxo-2-[(2R,5S)-2-(3-amino-4-sulfanyl-phenyl)-5-methyl-1-piperidyl]acetyl]amino]pyridine-3-carboxamide (0.3 g, 676.41 mol, 76.69% yield) was isolated.

LCMS(ESI): [M]⁺ m/z: calcd 443.2; found 444.2; Rt=1.165 min.

### Step 2: Synthesis of tert-butyl 4-(5-((2R,5S)-1-(2-((5-carbamoyl-6-methoxypyridin-3-yl)amino)-2-oxoacetyl)-5-methylpiperidin-2-yl)benzo[d]thiazol-2-yl)piperidine-1-carboxylate

Prepared by general procedure scheme 4.1 step 1B. Yield: 63 mg (14.63%).

HPLC conditions: Column: XBridge C18 100*19 mm, 5 microM; 0-5 min 45-70% water-MeCN+0.1% NH₄OH; (loading pump 4 ml/min MeOH).

LCMS(ESI): [M]⁺ m/z: calcd 636.2; found 637.2; Rt=3.934 min.

### Step 3: Synthesis of 2-methoxy-5-(2-((2R,5S)-5-methyl-2-(2-(piperidin-4-yl)benzo[d]thiazol-5-yl)piperidin-1-yl)-2-oxoacetamido)nicotinamide (Compound 52)

tert-Butyl 4-[5-[(2R,5S)-1-[2-[(5-carbamoyl-6-methoxy-3-pyridyl)amino]-2-oxoacetyl]-5-methyl-2-piperidyl]-1,3-benzothiazol-2-yl]piperidine-1-carboxylate (63 mg, 98.94 μmol) and TFA (148.00 mg, 1.30 mmol, 0.1 mL) were stirred in CHCl₃ (3 mL) for 16 h at 25° C. Upon completion, the reaction mixture was concentrated under reduced pressure. The obtained crude product was purified by reverse phase HPLC chromatography (Device (Mobile Phase, Column): SYSTEM 15-40% 0-5 min H₂O/MeCN/0.1% NH₄OH, flow: 30 ml/min (loading pump 4 ml/min MeCN) target mass 536.66 column: XBridge C18 100×19 mm, 5 um) to afford product 2-methoxy-5-[[2-oxo-2-[(2R,5S)-5-methyl-2-[2-(4-piperidyl)-1,3-benzothiazol-5-yl]-1-piperidyl]acetyl]amino]pyridine-3-carboxamide (21 mg, 39.13 μmol, 39.55% yield).

Compound 52: ¹H NMR (600 MHz, DMSO-d₆) δ (ppm) 0.99-1.09 (m, 3H), 1.29-1.42 (m, 1H), 1.67-1.77 (m, 3H), 1.82-1.95 (m, 1H), 2.01-2.20 (m, 3H), 2.20-2.36 (m, 1H), 2.60-2.77 (m, 2H), 2.83-2.87 (m, 0.3H), 2.98 (m, 1H), 3.04-3.11 (m, 2H), 3.18-3.23 (m, 1.7H), 3.47-3.51 (m, 0.7H), 3.90-3.97 (m, 3H), 4.01-4.06 (m, 0.3H), 5.25-5.76 (m, 1H), 7.33-7.45 (m, 1H), 7.64-7.79 (m, 2H), 7.84-7.91 (m, 1H), 8.02-8.11 (m, 1H), 8.34-8.62 (m, 2H), 10.86-11.23 (m, 1H).

LCMS(ESI): [M]⁺ m/z: calcd 536.2; found 537.2; Rt=2.540 min.

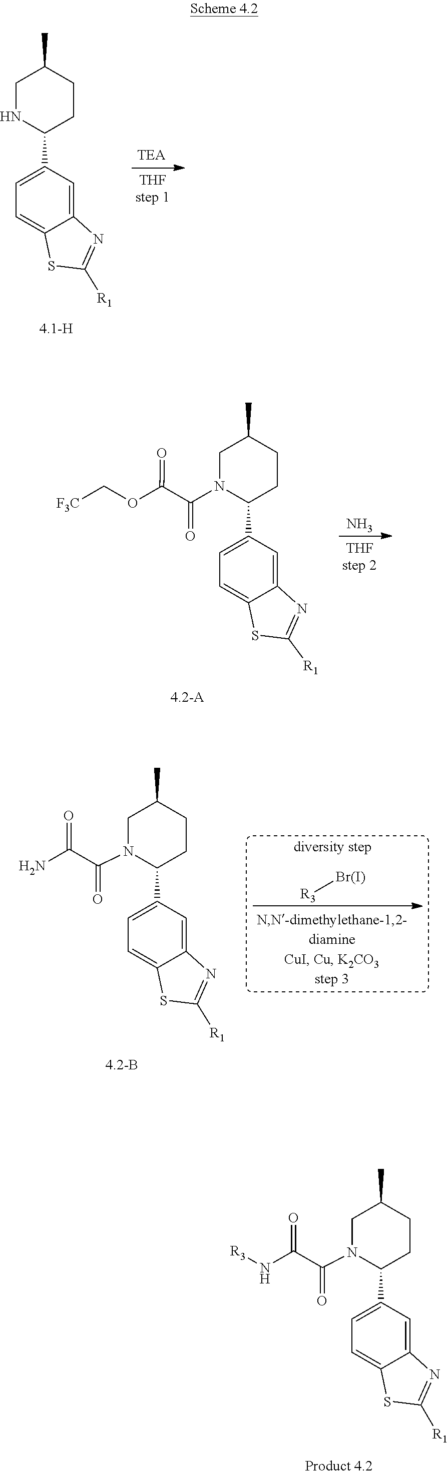

### Step 1: Synthesis of 4.2-A

4.1-H was described in scheme 4.1.

4.1-H (1 eq) and TEA (1.1 eq) were dissolved in THF and cooled to 0° C., following by the dropwise addition of 2,2,2-trifluoroethyl 2-chloro-2-oxo-acetate (1.1 eq) under Ar and the reaction mixture was stirred for 12 hr at rt and evaporated under reduced pressure to give 4.2-A which was used in the next step without further purification.

### Step 2: Synthesis of 4.2-B

To a solution of 4.2-A (1 eq) in THF (10 mL), ammonia (1 eq) was bubbled through for 10 min at 0° C. The reaction mixture was then stirred for 18 hr at rt. The reaction mixture was filtered off and the filtrate was evaporated in vacuum to give 4.2-B which was used in the next step without further purification.

### Step 3A: Synthesis of Product 4.2

4.2-B (1 eq), R₃Br (1.1 eq), Cu (1 eq), CuI (1 eq), K₂CO₃ (2 eq) and N,N-dimethylcyclohexane-1,2-diamine (1.5 eq) were mixed in dioxane under argon, and then stirred overnight at 95° C. for 24 hr in vial. The residue was purified by HPLC to obtain pure product.

### Example 60. Compound 8 2-fluoro-5-(2-((2R,5S)-5-methyl-2-(2-(1-methylpiperidin-4-yl)benzo[d]thiazol-5-yl)piperidin-1-yl)-2-oxoacetamido)nicotinamide

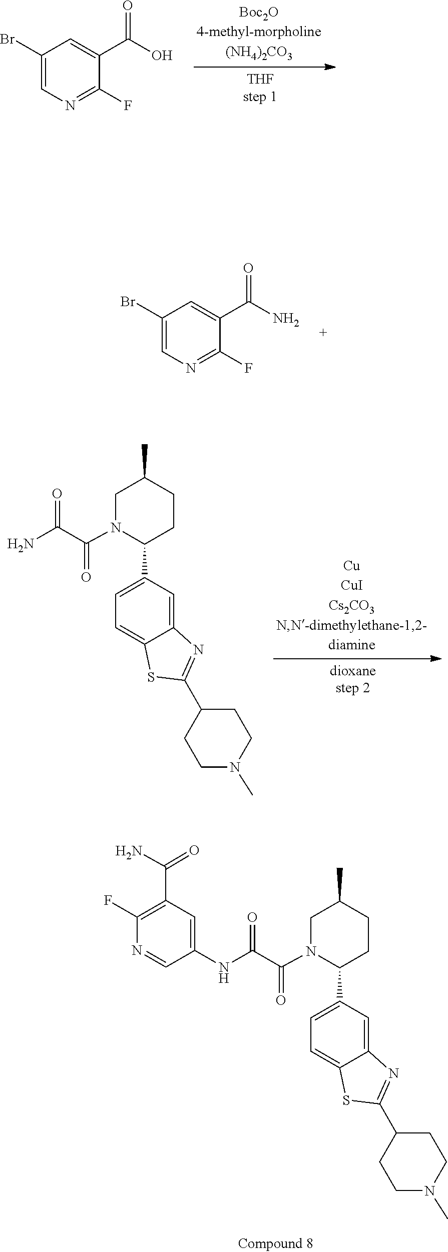 [SMILES: O=C(O)c1cc(Br)cnc1F ; NC(=O)c1cc(Br)cnc1F ; C[C@H]1CC[C@H](c2ccc3sc(C4CCN(C)CC4)nc3c2)N(C(=O)C(N)=O)C1 ; C[C@H]1CC[C@H](c2ccc3sc(C4CCN(C)CC4)nc3c2)N(C(=O)C(=O)Nc2cnc(F)c(C(N)=O)c2)C1]

The synthesis of 2-((2R,5S)-5-methyl-2-(2-(1-methylpiperidin-4-yl)benzo[d]thiazol-5-yl)piperidin-1-yl)-2-oxoacetamide is given by 3BBB, steps 1-6.

### Step 1: Synthesis of 5-bromo-2-fluoronicotinamide

To a solution 5-bromo-2-fluoro-pyridine-3-carboxylic acid (1.00 g, 4.55 mmol) and di-tert-butyl dicarbonate (1.39 g, 6.36 mmol, 1.46 mL) in THF (20 mL) was added 4-methyl-morpholine (597.70 mg, 5.91 mmol, 649.67 μL). The mixture was stirred for 30 min at 20° C. and then ammonium carbonate (1.43 g, 8.18 mmol) was added. The reaction mixture was stirred at 20° C. for 12 hr. Water (10 ml) and EtOAc (20 ml) was added and the mixture was stirred for 10 min. The organic layer was separated, washed with NaHCO₃ solution, brine, dried over Na₂SO₄ and evaporated to obtain 5-bromo-2-fluoro-pyridine-3-carboxamide (0.85 g, 3.88 mmol, 85.38% yield).

LCMS(ESI): [M]⁺ m/z: calcd 219.2; found 220.2; Rt=0.714 min.

### Step 2: Synthesis of 2-fluoro-5-(2-((2R,5S)-5-methyl-2-(2-(1-methylpiperidin-4-yl)benzo[d]thiazol-5-yl)piperidin-1-yl)-2-oxoacetamido)nicotinamide

Prepared by general procedure scheme 4.2 step 3A. Yield: 13 mg (2.64%).

HPLC conditions: Column: XBridge BEH C18 100*19 mm, 5 microM; 0-1-6 min 25-45% water-MeCN+0.1% NH₄OH, flow: 30 ml/min; (loading pump 4 ml/min MeCN).

Compound 8: ¹H NMR (600 MHz, DMSO-d₆) δ 1.02-1.06 (m, 3H), 1.31-1.39 (m, 1H), 1.66-1.73 (m, 1H), 1.75-1.95 (m, 4H), 2.01-2.10 (m, 5H), 2.18 (s, 3H), 2.28-2.34 (m, 1H), 2.81-2.85 (m, 2H), 3.02-3.09 (m, 1H), 3.44-4.06 (m, 1H), 5.25-5.78 (m, 1H), 7.35-7.44 (m, 1H), 7.78-7.92 (m, 3H), 8.01-8.09 (m, 1H), 8.38-8.62 (m, 2H), 10.62-12.13 (m, 1H).

LCMS(ESI): [M]⁺ m/z: calcd 538.2; found 539.2; Rt=2.392 min.

### Scheme 4.3—Synthesis of Compounds of Formula 10

Compounds of Formula 10 are compounds of Formula (I) wherein R¹, R², R³, R⁴, R⁶ R⁷, and R⁸ are as described herein.

### General Procedure 4.3

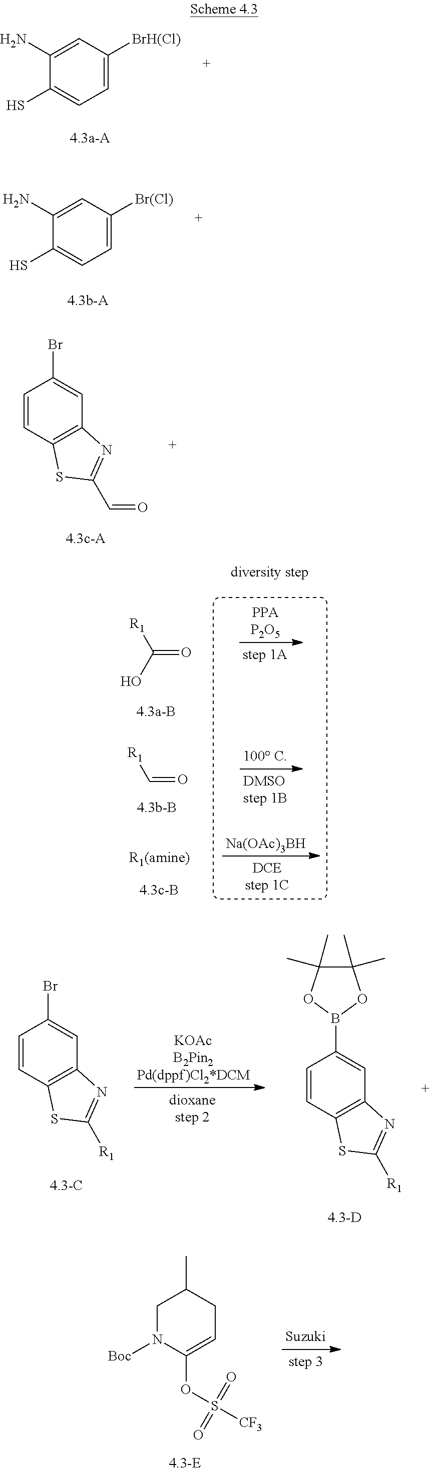

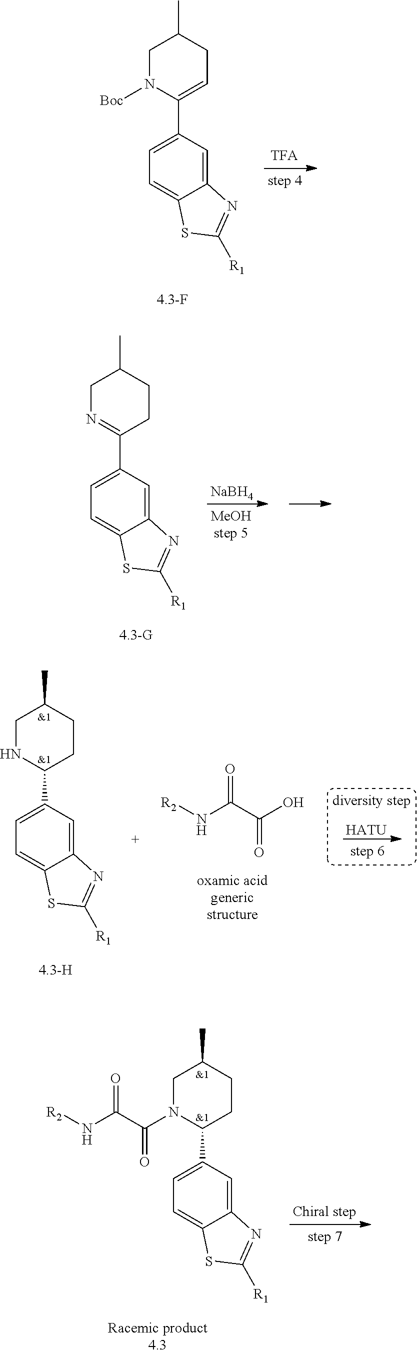

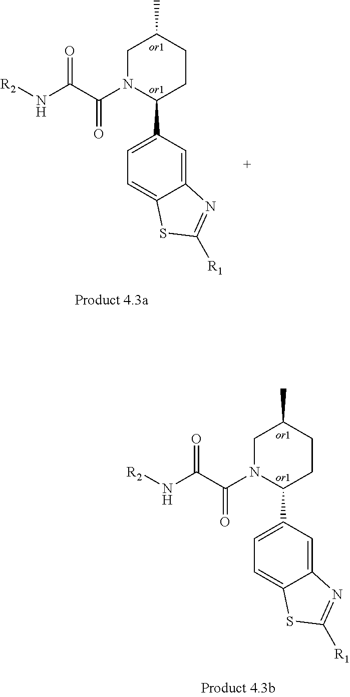

### Step 1A: Synthesis of 4.3-C

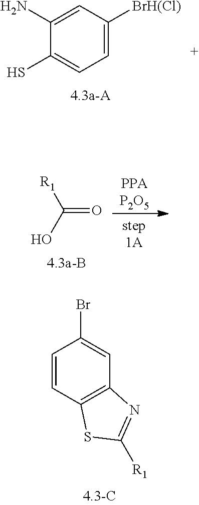

Phosphoric acid (4 eq) and phosphorus pentoxide (4 eq) were mixed together. The reaction suspension was stirred at rt for 10 min, then 4.3a-A (1 eq) followed by 4.3a-B (1.2 eq) were added under Ar. The solution was stirred at 110° C. for 18 hr then it was triturated with water, basidified (NaOH, 10% aq.) to pH=10, extracted with DCM twice, dried and evaporated in vacuum to give 4.3-C.

### Step 1B: Synthesis of 4.3-C

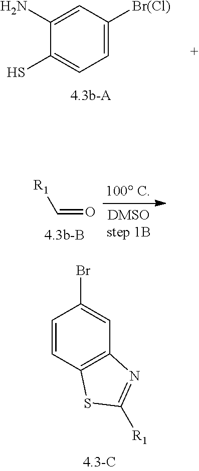

To the stirred solution of 4.3b-A (1 eq) in DMSO 4.1b-B (1 eq) was added. The resulting mixture was stirred at 100° C. for 14 hr. The reaction mixture was poured into cold water and extracted with MTBE twice. Combined organic layers were washed with water and brine, dried over Na₂SO₄. MTBE was evaporated in vacuum to give 4.3-C.

### Step 1C: Synthesis of 4.3-C

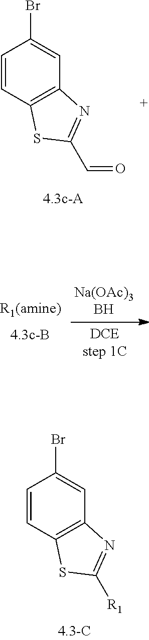

To the stirred solution of 4.3c-A (1 eq) in the 1,2-dichloroethane 4.3c-B (2 eq) was added and allowed to stir at 25° C. for 2 hr, sodium (trisacetoxy) borohydride (2 eq) was added. The reaction mixture was stirred at 25° C. for 16 hr. After completion, the reaction mixture was evaporated, quenched with water and neutralized by K₂CO₃ to pH=10. The aqueous phase was extracted with CHCl₃ twice. The combined organic phase was dried over Na₂SO₄ and evaporated under reduced pressure to afford 4.3-C. (TEA 1.5 eq per each acid eq, if amine salt used, was added to the solution of respective amine) Step 2: Synthesis of 4.3-D

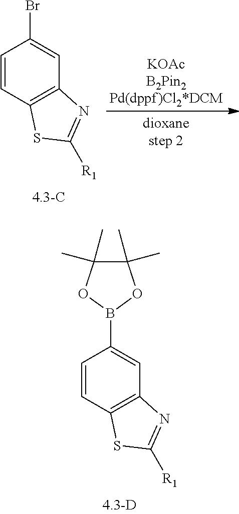

4.3-C(1 eq), B₂Pin₂ (1.1 eq) and KOAc (2 eq) were mixed in dioxane. The resulting mixture was evacuated and then backfilled with argon, this operation was repeated three times, then Pd(dppf)Cl₂*DCM (0.05 eq) was added under argon. The reaction mixture was stirred under argon at 90° C. for 14 hr, then cooled and filtered. The filter cake was washed with dioxane twice. The solvent was evaporated to afford 4.3-D.

### Step 3: Synthesis of 4.3-F

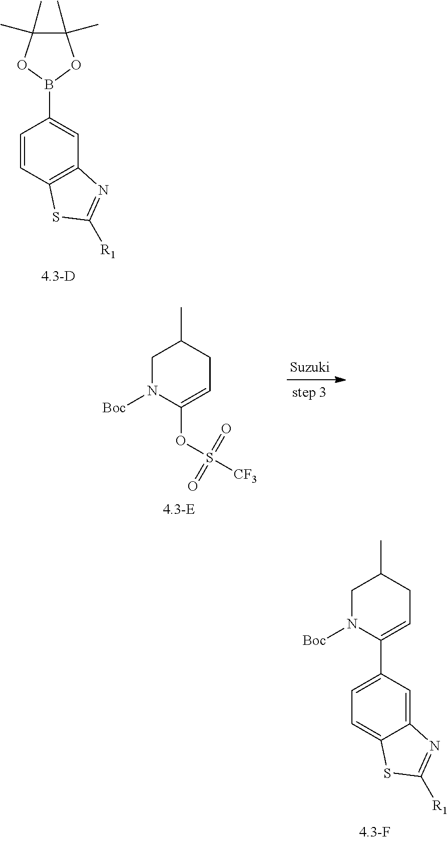

4.3-D (1 eq), tert-butyl 3-methyl-6-(trifluoromethylsulfonyloxy)-3,4-dihydro-2H-pyridine-1-carboxylate (1.2 eq), sodium carbonate (3 eq) were mixed together in dioxane-water mixture (3:1). The resulting mixture was evacuated and then backfilled with argon. This operation was repeated two times, then Pd(dppf)Cl₂*DCM (819.86 mg, 1.00 mmol) was added and the reaction mixture was stirred under argon at 90° C. overnight, then cooled down and concentrated in vacuum. The residue was diluted with MTBE and stirred for 0.5 hr. After the most of the residue had dissolved, anhydrous sodium sulphate was added, and the resulting mixture was filtered. The filter cake was additionally washed with MTBE (5*50 ml) and discarded. The filtrate was concentrated in vacuum to afford 4.3-F.

### Step 4: Synthesis of 4.3-G

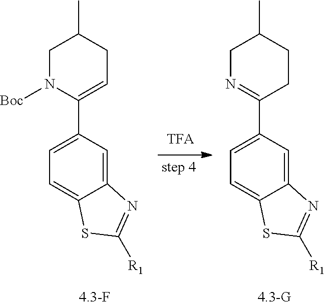

A solution of 4.3-F (1 eq) in TFA (15 eq) was stirred at rt for 1 hr, and then concentrated in vacuum. Cold water was added to the residue, and the resulting mixture was extracted with DCM twice. The DCM layer was discarded, and the aqueous layer was basified to pH 11. The resulting mixture was extracted with DCM twice. The combined organic extracts were dried over sodium sulphate and concentrated in vacuum to afford 4.3-G.

### Step 5: Synthesis of 4.3-H

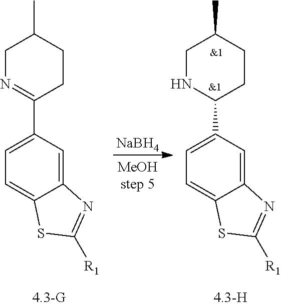

4.3-G (1 eq) was dissolved in MeOH and the resulting solution was cooled to 0° C. in an ice bath. Sodium borohydride (2 eq) was added portion wise to the previous solution. After addition completed, the reaction mixture was allowed to warm to rt and stirred overnight. Water was added to the reaction mixture and the resulting mixture was concentrated in vacuum. The residue was diluted with water and the resulting mixture was extracted with DCM twice, dried over Na₂SO₄, filtered and evaporated to obtain 4.3-H.

### Step 6A: Synthesis of Racemic Product 4.3

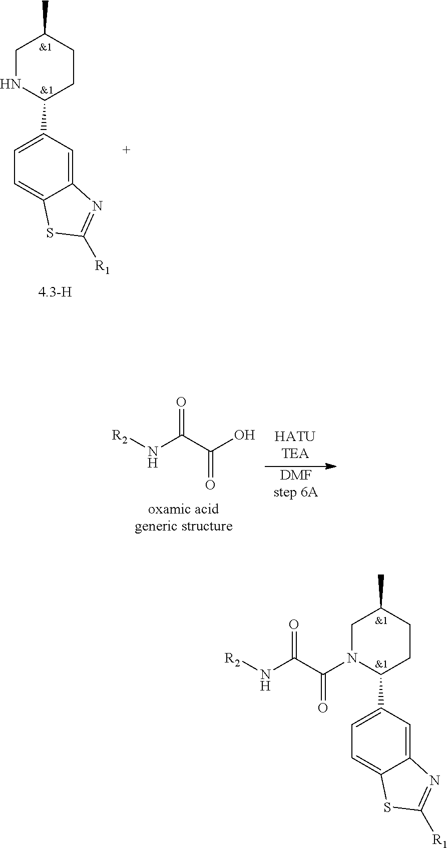

4.3-H (1 eq), oxamic acid (1 eq) and TEA (2.5 eq+1.0 eq per each acid eq, if amine salt used) were mixed together in DMF. HATU (1.5 eq) was added thereto and the resulting mixture was stirred overnight. The reaction mixture was concentrated in vacuum and the residue was purified by HPLC to obtain racemic product 4.3.

### Step 6B: Synthesis of Racemic Product 4.3

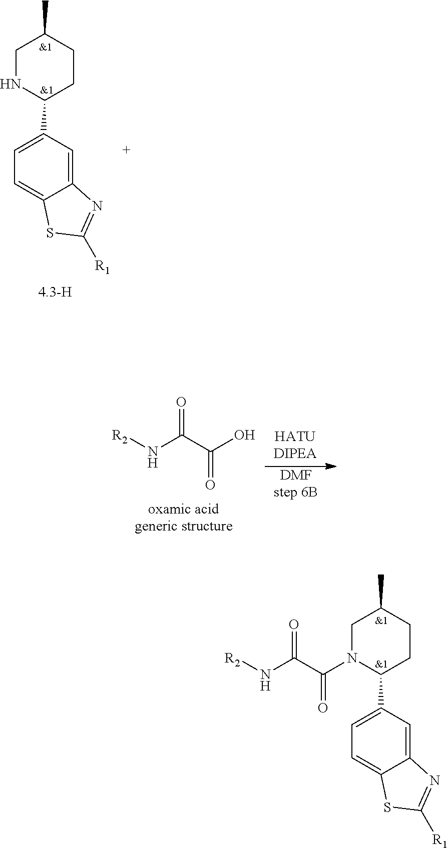

DIPEA (2.5 eq+1.0 eq per each acid eq, if amine salt used) was added to the solution of respective amine or it salt (4.3-H) (1 eq) and oxamic acid (1 eq) in DMF. The resulting mixture was stirred for 5 min followed by the addition of the solution of HATU (1.1 eq) in DMF. Then, the reaction mixture was stirred overnight at rt. After the completion of the reaction, monitored by LCMS, the resulting suspension was concentrated under reduced pressure. The obtained filtrate was subjected to HPLC (Waters SunFire C18 19*100 5 mkm column and H₂O-MeOH as a mobile phase) to afford pure product.

### Example 61. Compound 68 and Compound 35 2-methoxy-5-(2-((2R,5S)-2-(2-(rac-(3S,4R)-3-methoxy-1-methylpiperidin-4-yl)benzo[d]thiazol-5-yl)-5-methylpiperidin-1-yl)-2-oxoacetamido)nicotinamide

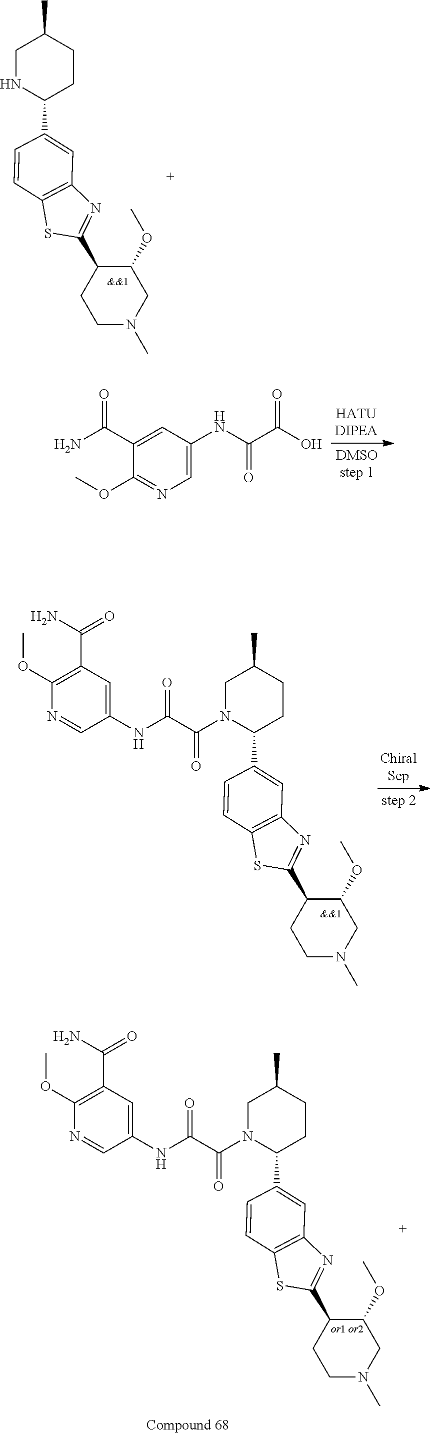 [SMILES: CO[C@@H]1CN(C)CC[C@H]1c1nc2cc([C@H]3CC[C@H](C)CN3)ccc2s1 ; COc1ncc(NC(=O)C(=O)O)cc1C(N)=O ; COc1ncc(NC(=O)C(=O)N2C[C@@H](C)CC[C@@H]2c2ccc3sc([C@@H]4CCN(C)C[C@H]4OC)nc3c2)cc1C(N)=O ; COc1ncc(NC(=O)C(=O)N2C[C@@H](C)CC[C@@H]2c2ccc3sc([C@@H]4CCN(C)C[C@H]4OC)nc3c2)cc1C(N)=O]

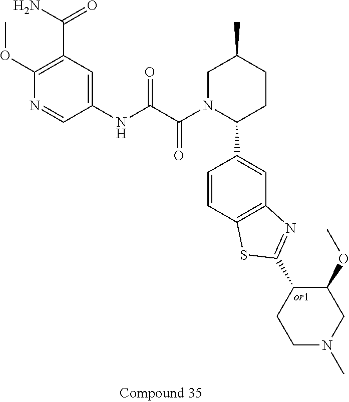 [SMILES: COc1ncc(NC(=O)C(=O)N2C[C@@H](C)CC[C@@H]2c2ccc3sc([C@H]4CCN(C)C[C@@H]4OC)nc3c2)cc1C(N)=O]

The synthesis of 2-(rac-(3S,4R)-3-methoxy-1-methylpiperidin-4-yl)-5-((2R,5S)-5-methylpiperidin-2-yl)benzo[d]thiazole is given by 3111.

### Step 1: Synthesis of 2-methoxy-5-(2-((2R,5S)-2-(2-(rac-(3S,4R)-3-methoxy-1-methylpiperidin-4-yl)benzo[d]thiazol-5-yl)-5-methylpiperidin-1-yl)-2-oxoacetamido)nicotinamide

Prepared by general procedure scheme 4.3 step 6A. Yield: 47 mg (14.55%).

HPLC conditions: Column: SunFire C18 100*19 mm, 5 microM; 2-10 min 40-95% MeOH+NH₃, flow: 30 ml/min; (loading pump 4 ml/min MeOH).

LCMS(ESI): [M]⁺ m/z: calcd 580.2; found 581.2; Rt=1.036 min.

### Step 2: Chiral Separation (Compound 68 and Compound 35)

Racemic 2-methoxy-5-[[2-oxo-2-[(2R,5S)-5-methyl-2-[2-[rac-(3S,4R)-3-methoxy-1-methyl-4-piperidyl]-1,3-benzothiazol-5-yl]-1-piperidyl]acetyl]amino]pyridine-3-carboxamide (0.047 g, 80.94 mol) was chiral separated (Column: Chiralcel OD-H (250*20 mm, 5 mkm); Mobile phase: Hexane-IPA-MeOH, 80-10-10. Flow Rate: 14 mL/min) to obtain 2-methoxy-5-[[2-oxo-2-[(2R,5S)-5-methyl-2-[2-[(3S,4R)-3-methoxy-1-methyl-4-piperidyl]-1,3-benzothiazol-5-yl]-1-piperidyl]acetyl]amino]pyridine-3-carboxamide (0.013 g, 22.39 mol, 27.66% yield) and 2-methoxy-5-[[2-oxo-2-[(2R,5S)-5-methyl-2-[2-[(3R,4S)-3-methoxy-1-methyl-4-piperidyl]-1,3-benzothiazol-5-yl]-1-piperidyl]acetyl]amino]pyridine-3-carboxamide (0.0129 g, 22.21 mol, 27.45% yield).

Rel Time for Compound 68 in analytical conditions (column: OD-H, Hexane-IPA-MeOH, 70-15-15, 0.6 ml/min as mobile phase) 37.43 min and for Compound 35 44.54 min.

### Compound 68: Retention Time: 37.43 Min

1H NMR (600 MHz, dmso) δ 1.01-1.07 (m, 3H), 1.30-1.44 (m, 1H), 1.65-1.76 (m, 1H), 1.83-1.95 (m, 2H), 2.05-2.19 (m, 4H), 2.20 (s, 3H), 2.27-2.36 (m, 1H), 2.69-3.21 (m, 4H), 3.21 (s, 3H), 3.47-3.55 (m, 0,7H), 3.70-3.77 (m, 1H), 3.89-4.00 (m, 3H), 4.02-4.07 (m, 0,3H), 5.25-5.76 (m, 1H), 7.31-7.44 (m, 1H), 7.66-7.78 (m, 2H), 7.82-7.91 (m, 1H), 8.00-8.09 (m, 1H), 8.40-8.49 (m, 1H), 8.49-8.62 (m, 1H), 10.88-11.14 (m, 1H).

LCMS(ESI): [M]⁺ m/z: calcd 580.2; found 581.2; Rt=2.238 min.

### Compound 35: Retention Time: 44.54 Min

1H NMR (600 MHz, dmso) δ 1.02-1.07 (m, 3H), 1.32-1.43 (m, 1H), 1.67-1.79 (m, 1H), 1.83-1.92 (m, 2H), 2.01-2.15 (m, 3H), 2.18 (s, 3H), 2.18-2.32 (m, 2H), 2.32-2.37 (m, 1H), 2.70-2.79 (m, 1H), 2.80-2.85 (m, 0,3H), 2.93-3.06 (m, 1H), 3.19-3.23 (m, 3H), 3.25-3.27 (m, 0,7H), 3.49-3.53 (m, 0,7H), 3.71-3.76 (m, 1H), 3.90-3.99 (m, 3H), 4.03-4.07 (m, 0,3H), 5.25-5.74 (m, 1H), 7.31-7.46 (m, 1H), 7.63-7.78 (m, 2H), 7.85-7.92 (m, 1H), 8.00-8.08 (m, 1H), 8.38-8.49 (m, 1H), 8.49-8.63 (m, 1H), 10.86-11.29 (m, 1H).

LCMS(ESI): [M]⁺ m/z: calcd 580.2; found 581.2; Rt=2.242 min.

### Example 62. Compound 82 and Compound 67 2-methoxy-5-(2-((2R,5S)-5-methyl-2-(2-((3aR,6aS)-2-methyloctahydrocyclopenta[c]pyrrol-5-yl)benzo[d]thiazol-5-yl)piperidin-1-yl)-2-oxoacetamido)nicotinamide

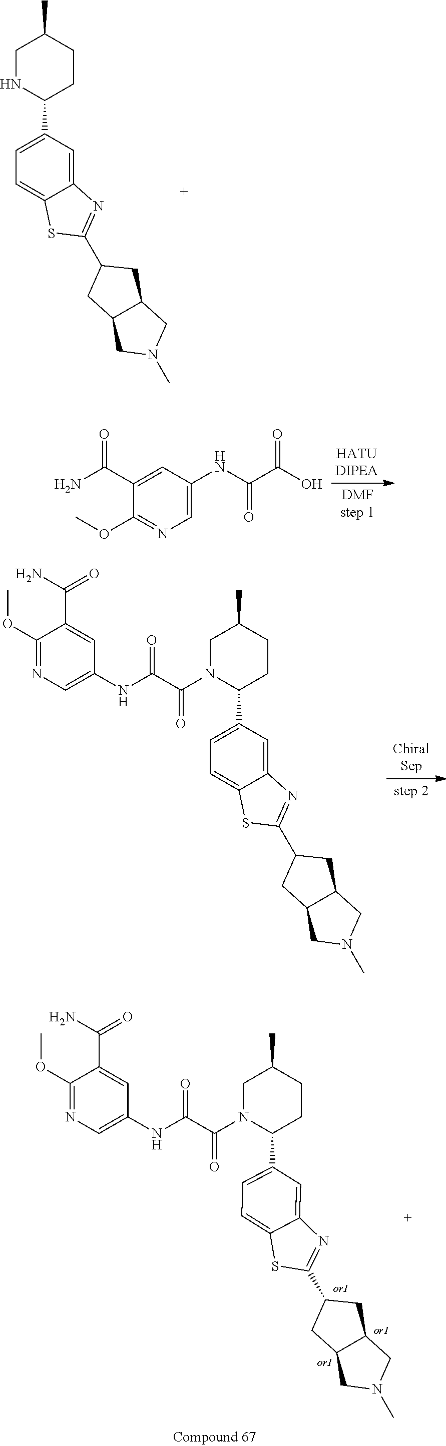

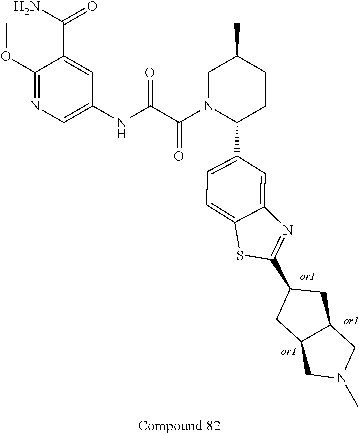 [SMILES: COc1ncc(NC(=O)C(=O)N2C[C@@H](C)CC[C@@H]2c2ccc3sc([C@@H]4C[C@@H]5CN(C)C[C@@H]5C4)nc3c2)cc1C(N)=O]

The synthesis of 5-((S)-5-methyl-3,4,5,6-tetrahydropyridin-2-yl)-2-(rac-(3aR,5s,6aS)-2-methyloctahydrocyclopenta[c]pyrrol-5-yl)benzo[d]thiazole is given by 3000.

### Step 1: Synthesis of 2-methoxy-5-(2-((2R,5S)-5-methyl-2-(2-((3aR,6aS)-2-methyloctahydrocyclopenta[c]pyrrol-5-yl)benzo[d]thiazol-5-yl)piperidin-1-yl)-2-oxoacetamido)nicotinamide

Prepared by general procedure scheme 4.3 step 6B. Yield: 56 mg (23.23%).

HPLC conditions: Column: SunFire C18 100*19 mm, 5 microM; 2-10 min 30% water-MeCN+FA, flow: 30 ml/min; (loading pump 4 ml/min MeCN).

LCMS(ESI): [M]⁺ m/z: calcd 576.2; found 577.2; Rt=2.565 min.

### Step 2: Chiral Separation (Compound 82 and Compound 67)

Racemic 2-methoxy-5-[[2-oxo-2-[(2R,5S)-5-methyl-2-[2-[(3aR,6aS)-2-methyl-3,3a,4,5,6,6a-hexahydro-1H-cyclopenta[c]pyrrol-5-yl]-1,3-benzothiazol-5-yl]-1-piperidyl]acetyl]amino]pyridine-3-carboxamide (56.00 mg, 97.10 mol) was chiral separated (Column: Chiralcel OJ-H-I (250*20, 5 mkm); Hexane-IPA-MeOH, 70-15-15; 12 ml/min) to obtain 2-methoxy-5-(2-((2R,5S)-5-methyl-2-(2-((3aR,5s,6aS)-2-methyloctahydrocyclopenta[c]pyrrol-5-yl)benzo[d]thiazol-5-yl)piperidin-1-yl)-2-oxoacetamido)nicotinamide (5.8 mg, 10.06 mol, 10.36% yield) and 2-methoxy-5-(2-((2R,5S)-5-methyl-2-(2-((3aR,5r,6aS)-2-methyloctahydrocyclopenta[c]pyrrol-5-yl)benzo[d]thiazol-5-yl)piperidin-1-yl)-2-oxoacetamido)nicotinamide (7.3 mg, 12.66 μmol, 13.04% yield).

Rel Time for Compound 82 in analytical conditions (column: IB, CO₂-MeOH, 70-30, 2 ml/min as mobile phase) 37.85 min and for Compound 67 30.29 min.

### Compound 82: Retention Time: 37.85 Min

¹H NMR (600 MHz, DMSO-d₆) δ (ppm) 1.00-1.11 (m, 3H), 1.19-1.29 (m, 2H), 1.32-1.45 (m, 1H), 1.63-1.77 (m, 1H), 1.82-1.95 (m, 1H), 1.99-2.04 (m, 3H), 2.04-2.24 (m, 2H), 2.24-2.36 (m, 1H), 2.38-2.47 (m, 3H), 2.73-2.88 (m, 3H), 2.89-3.21 (m, 2H), 3.49-3.55 (m, 0.7H), 3.76-3.87 (m, 1H), 3.89-3.99 (m, 3H), 4.02-4.06 (m, 0.3H), 5.18-5.83 (m, 1H), 7.28-7.46 (m, 1H), 7.66-7.79 (m, 2H), 7.82-7.91 (m, 1H), 8.02-8.11 (m, 1H), 8.37-8.63 (m, 2H), 10.92-11.41 (m, 1H).

LCMS(ESI): [M]⁺ m/z: calcd 576.2; found 577.2; Rt=1.612 min.

### Compound 67: Retention Time: 30.29 Min

¹H NMR (600 MHz, DMSO-d₆) δ (ppm) 0.84-1.06 (m, 3H), 1.20-1.23 (m, 3H), 1.32-1.42 (m, 1H), 1.43-1.65 (m, 1H), 1.66-1.83 (m, 3H), 1.82-1.92 (m, 1H), 2.04-2.28 (m, 2H), 2.28-2.35 (m, 1H), 2.39-2.44 (m, 2H), 2.70-3.08 (m, 5H), 3.47-3.54 (m, 1.7H), 3.91-4.00 (m, 3H), 4.02-4.06 (m, 0.3H), 5.22-5.75 (m, 1H), 7.32-7.45 (m, 1H), 7.65-7.78 (m, 2H), 7.82-7.91 (m, 1H), 7.97-8.11 (m, 1H), 8.38-8.62 (m, 2H), 11.01-11.18 (m, 1H).

LCMS(ESI): [M]⁺ m/z: calcd 576.2; found 577.2; Rt=1.609 min.

### Example 63. Compound 111 and Compound 33 N-(6-amino-5-ethylpyridin-3-yl)-2-((2R,5S)-5-methyl-2-(2-((3aR,6aS)-2-methyloctahydrocyclopenta[c]pyrrol-5-yl)benzo[d]thiazol-5-yl)piperidin-1-yl)-2-oxoacetamide

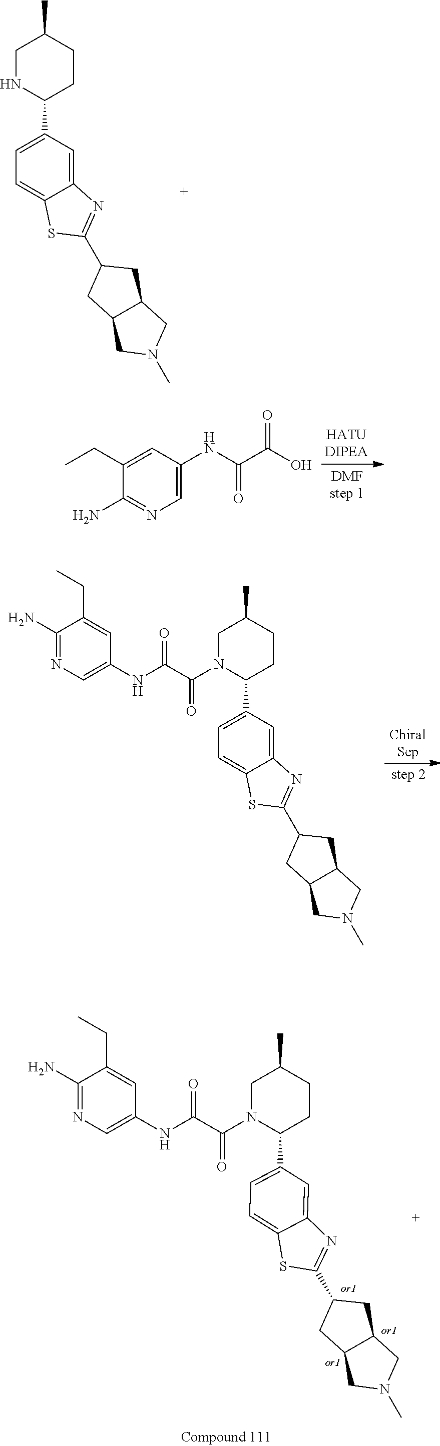

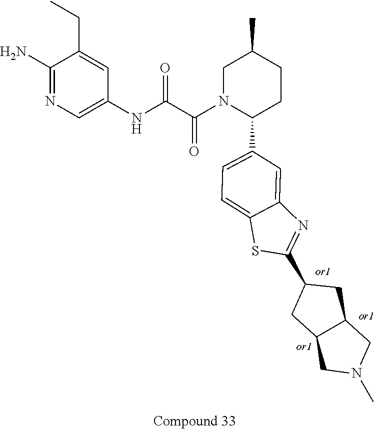 [SMILES: CCc1cc(NC(=O)C(=O)N2C[C@@H](C)CC[C@@H]2c2ccc3sc([C@@H]4C[C@@H]5CN(C)C[C@@H]5C4)nc3c2)cnc1N]

The synthesis of 5-((S)-5-methyl-3,4,5,6-tetrahydropyridin-2-yl)-2-(rac-(3aR,5s,6aS)-2-methyloctahydrocyclopenta[c]pyrrol-5-yl)benzo[d]thiazole is given by 3000.

### Step 1: Synthesis of N-(6-amino-5-ethylpyridin-3-yl)-2-((2R,5S)-5-methyl-2-(2-((3aR,6aS)-2-methyloctahydrocyclopenta[c]pyrrol-5-yl)benzo[d]thiazol-5-yl)piperidin-1-yl)-2-oxoacetamide

Prepared by general procedure scheme 4.3 step 6B. Yield: 80 mg (30.61%).

HPLC conditions: Column: SunFire C18 100*19 mm, 5 microM; 0-5 min 5-55% water-MeOH+NH₃, flow: 30 ml/min; (loading pump 4 ml/min MeOH).

LCMS(ESI): [M]⁺ m/z: calcd 546.2; found 547.2; Rt=0.952 min.

### Step 2: Chiral Separation (Compound 111 and Compound 33)

Racemic N-(6-amino-5-ethyl-3-pyridyl)-2-oxo-2-[(2R,5S)-5-methyl-2-[2-[(3aR,6aS)-2-methyl-3,3a,4,5,6,6a-hexahydro-1H-cyclopenta[c]pyrrol-5-yl]-1,3-benzothiazol-5-yl]-1-piperidyl]acetamide (80 mg, 146.33 μmol) was chiral separated (Column: Chiralcel OD-H (250*20, 5 mkm); Hexane-IPA-MeOH, 80-10-10; Flow rate: 13 ml/min; RT1=36.39 min, RT2=39.21 min and Chiralcel OD-H (250*20, 5 mkm); Hexane-IPA-MeOH, 70-15-15; Flow rate: 12 ml/min; RT1=23.32 min, RT2=25.52 min were purification from cis-isomer; Chiralpak IA III (250*30, 10 mkm); MeOH-IPA, 80-20; Flow rate: 12 ml/min; RT1=12.9 min, RT2=20.56 min was separation of enantiomers) to obtain N-(6-amino-5-ethylpyridin-3-yl)-2-((2R,5S)-5-methyl-2-(2-((3aR,5s,6aS)-2-methyloctahydrocyclopenta[c]pyrrol-5-yl)benzo[d]thiazol-5-yl)piperidin-1-yl)-2-oxoacetamide (13.02 mg, 23.81 μmol, 16.28% yield) and N-(6-amino-5-ethylpyridin-3-yl)-2-((2R,5S)-5-methyl-2-(2-((3aR,5r,6aS)-2-methyloctahydrocyclopenta[c]pyrrol-5-yl)benzo[d]thiazol-5-yl)piperidin-1-yl)-2-oxoacetamide (14.62 mg, 26.74 mol, 18.28% yield).

Rel Time for Compound 111 in analytical conditions (column: IB, CO₂-MeOH, 70-30, 2 ml/min as mobile phase) 35.02 min and for Compound 33 26.41 min.

### Compound 111: Retention Time: 35.02 Min

¹H NMR (500 MHz, DMSO-d₆) δ (ppm) 1.01-1.05 (m, 3H), 1.11-1.19 (m, 3H), 1.31-1.40 (m, 1H), 1.64-1.78 (m, 1H), 1.83-1.93 (m, 1H), 1.94-2.01 (m, 1H), 2.02-2.12 (m, 2H), 2.14-2.25 (m, 1H), 2.38-2.43 (m, 4H), 2.75-2.85 (m, 4H), 2.91-3.10 (m, 2H), 3.47-3.56 (m, 2H), 3.73-3.78 (m, 1H), 3.86-4.12 (m, 2H), 5.30-5.70 (m, 1H), 7.33-7.42 (m, 1H), 7.42-8.03 (m, 4H), 8.03-8.13 (m, 1H), 8.17-8.36 (m, 1H), 9.18-9.59 (m, 1H), 10.89-11.28 (m, 1H).

LCMS(ESI): [M]⁺ m/z: calcd 546.2; found 547.2; Rt=2.974 min.

### Compound 33: Retention Time: 26.41 Min

¹H NMR (500 MHz, DMSO-d₆) δ (ppm) 0.85-1.17 (m, 9H), 1.30-1.43 (m, 2H), 1.66-1.74 (m, 1H), 1.77-1.82 (m, 1H), 1.83-1.94 (m, 2H), 2.02-2.14 (m, 1H), 2.77-2.86 (m, 4H), 2.88-2.99 (m, 2H), 2.99-3.06 (m, 2H), 3.08-3.16 (m, 2H), 3.48-3.53 (m, 2H), 3.82-4.04 (m, 1H), 5.29-5.73 (m, 1H), 7.12-7.46 (m, 2H), 7.71-7.88 (m, 2H), 7.98-8.12 (m, 1H), 8.16-8.39 (m, 1H), 9.68 (s, 1H), 10.79-11.21 (m, 1H).

LCMS(ESI): [M]⁺ m/z: calcd 546.2; found 547.2; Rt=3.016 min.

### Example 64. Compound 96 and Compound 25 5-(2-((2R,5S)-2-(2-(1-(dimethylamino)ethyl)benzo[d]thiazol-5-yl)-5-methylpiperidin-1-yl)-2-oxoacetamido)-2-methoxynicotinamide

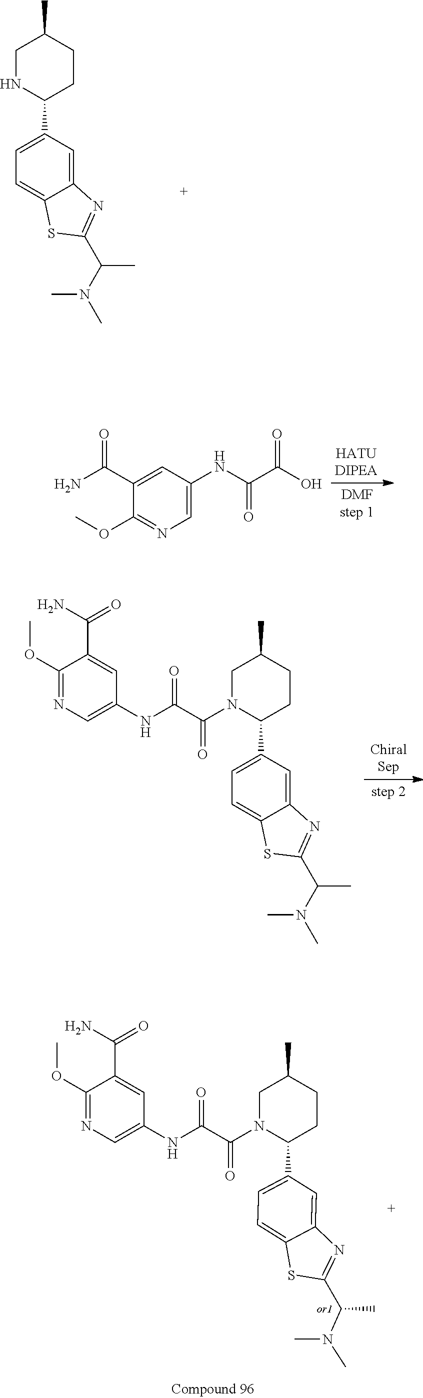 [SMILES: CC(c1nc2cc([C@H]3CC[C@H](C)CN3)ccc2s1)N(C)C ; COc1ncc(NC(=O)C(=O)O)cc1C(N)=O ; COc1ncc(NC(=O)C(=O)N2C[C@@H](C)CC[C@@H]2c2ccc3sc(C(C)N(C)C)nc3c2)cc1C(N)=O ; COc1ncc(NC(=O)C(=O)N2C[C@@H](C)CC[C@@H]2c2ccc3sc([C@H](C)N(C)C)nc3c2)cc1C(N)=O]

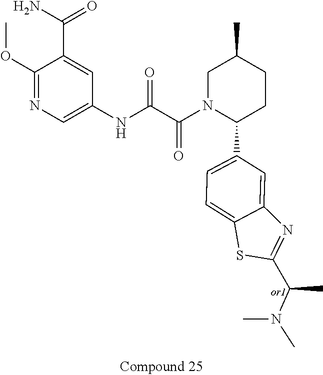 [SMILES: COc1ncc(NC(=O)C(=O)N2C[C@@H](C)CC[C@@H]2c2ccc3sc([C@@H](C)N(C)C)nc3c2)cc1C(N)=O]

The synthesis of N,N-dimethyl-1-(5-((2R,5S)-5-methylpiperidin-2-yl)benzo[d]thiazol-2-yl)ethanamine is given by 3GGG.

### Step 1: Synthesis of 5-(2-((2R,5S)-2-(2-(1-(dimethylamino)ethyl)benzo[d]thiazol-5-yl)-5-methylpiperidin-1-yl)-2-oxoacetamido)-2-methoxynicotinamide

Prepared by general procedure scheme 4.3 step 6A. Yield: 97 mg (28.05%).

HPLC conditions: Column: Chromatorex C18 100*19 mm, 5 microM; 0-1-6 min 35-35-60% water-MeCN+0.1% NH₄OH, flow: 30 ml/min; (loading pump 4 ml/min MeCN).

LCMS(ESI): [M]⁺ m/z: calcd 524.2; found 525.2; Rt=0.759 min.

### Step 2: Chiral Separation (Compound 96 and Compound 25)

Racemic 2-methoxy-5-[[2-oxo-2-[(2R,5S)-2-[2-[1-(dimethylamino)ethyl]-1,3-benzothiazol-5-yl]-5-methyl-1-piperidyl]acetyl]amino]pyridine-3-carboxamide (97 mg, 184.89 mol) was chiral separated (Column: CHIRALCEL OD-H (250×20 mm, 5 mkm), Mobile Phase: Hexane:IPA:MeOH, 70:15:15, Flow Rate: 15 ml/min) to obtain 2-methoxy-5-[[2-oxo-2-[(2R,5S)-5-methyl-2-[2-[(1S)-1-(dimethylamino)ethyl]-1,3-benzothiazol-5-yl]-1-piperidyl]acetyl]amino]pyridine-3-carboxamide (37 mg, 70.53 mol, 76.29% yield) (RT=19.25 min) and 2-methoxy-5-[[2-oxo-2-[(2R,5S)-5-methyl-2-[2-[(1R)-1-(dimethylamino)ethyl]-1,3-benzothiazol-5-yl]-1-piperidyl]acetyl]amino]pyridine-3-carboxamide (33 mg, 62.90 mol, 68.04% yield) (RT=24.95 min).

Rel Time for Compound 96 in analytical conditions (column: OD-H, Hexane-IPA-MeOH, 50-25-25, 0.6 ml/min as mobile phase) 11.06 min and for Compound 25 13.21 min.

### Compound 96: Retention Time: 11.06 Min

¹H NMR (600 MHz, DMSO-d₆) δ 1H NMR (DMSO-d6, 600 MHz): δ (ppm) 1.02-1.41 (m, 8H), 1.71-2.27 (m, 9H), 2.82-2.85 (m, 1H), 3.49-3.51 (m, 1H), 3.91-4.06 (m, 3H), 5.28-5.70 (m, 2H), 7.34-7.41 (m, 1H), 7.68-7.74 (m, 2H), 7.87-7.89 (m, 1H), 8.01-8.05 (m, 1H), 8.42-8.58 (m, 2H), 11.02-11.11 (m, 1H).

LCMS(ESI): [M]⁺ m/z: calcd 524.2; found 525.2; Rt=2.463 min.

### Compound 25: Retention Time: 13.21 Min

¹H NMR (600 MHz, DMSO-d₆) δ (ppm) 1.04-1.41 (m, 8H), 1.69-2.31 (m, 9H), 2.84-2.86 (m, 1H), 3.49-3.51 (m, 1H), 3.91-4.06 (m, 3H), 5.28-5.70 (m, 2H), 7.35-7.41 (m, 1H), 7.68-7.74 (m, 2H), 7.86-7.89 (m, 1H), 8.01-8.05 (m, 1H), 8.40-8.58 (m, 2H), 11.02-11.11 (m, 1H).

LCMS(ESI): [M]⁺ m/z: calcd 524.2; found 525.2; Rt=2.451 min.

### Scheme 6.2—Synthesis of Compounds of Formula 13

Compounds of Formula 11 are compounds of Formula (I) wherein R¹, R², R³, R⁴, R⁶ R⁷, and R⁸ are as described herein.

### General Procedure 6.2

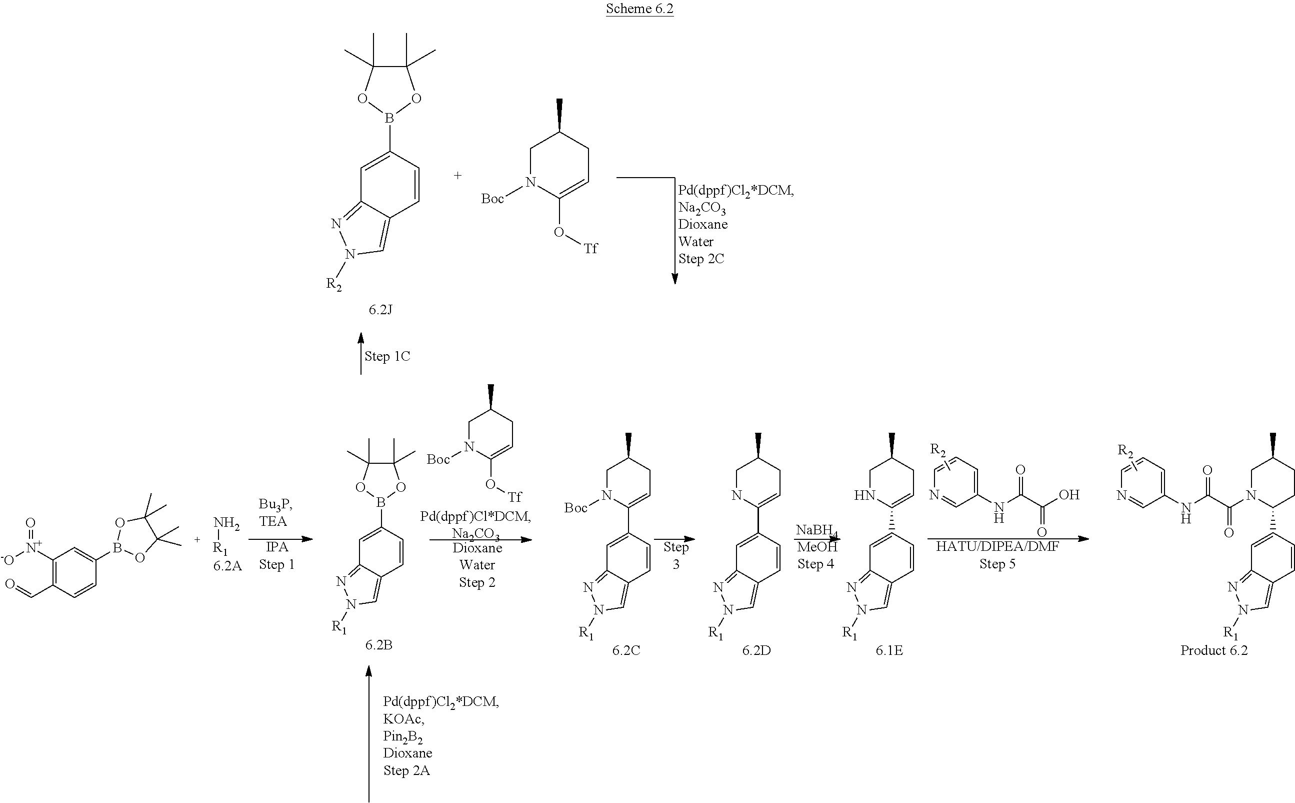

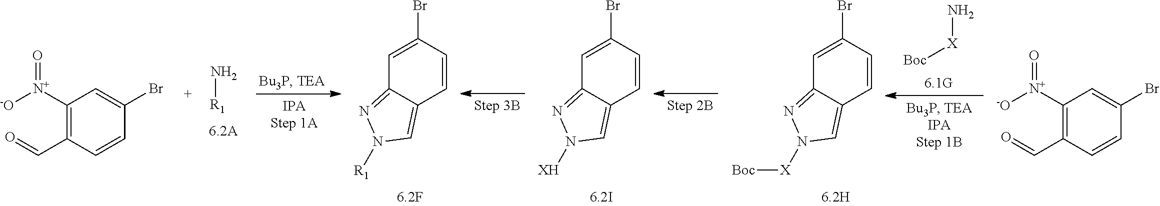

### Step 1: General Procedure for 6.2B

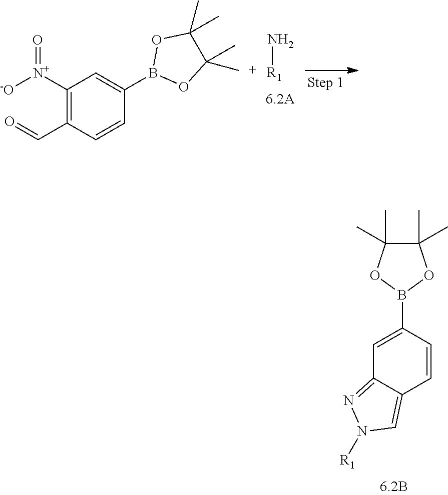

2-Nitro-5-(4,4,5,5-tetramethyl-1,3,2-dioxaborolan-2-yl)benzaldehyde (1.0 equiv.) and 6.2A (1.0 equiv., or it's salt) were dissolved in i-PrOH (140 mL). The resulting mixture was stirred at 80° C. for 2 hours following by the addition of tri-n-butyl phosphine (Bu₃P, 3.0 equiv.). The reaction mixture was stirred under reflux for 2 hours. Then, the volatiles were removed in vacuo. The residue was dissolved in DCM and washed with water. The organic layer separated, dried over Na₂SO₄ and concentrated under reduced pressure. The residue was purified by flash chromatography to afford 6.2B.

### Step 1A: General Procedure for 6.2F

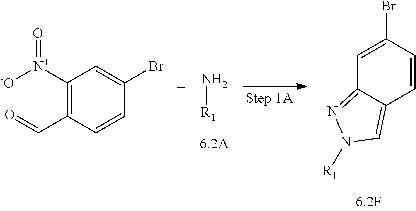

5-Bromo-2-nitro-benzaldehyde (1.0 equiv.) and 6.2A (1.0 equiv.) were dissolved in i-PrOH (400.0 mL). The resulting mixture was stirred at 80° C. for 2 hr following by the addition of tri-n-butyl phosphine (3.0 equiv.). The reaction mixture was refluxed additionally for 16 hr. Then the reaction mixture was evaporated under reduced pressure and purified by flash chromatography to afford 6.2F.

### Step 1B: General Procedure for 6.2H

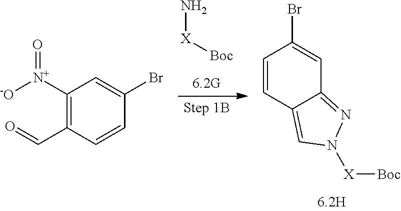

5-Bromo-2-nitro-benzaldehyde (1.0 equiv.) and 6.2G (1.0 equiv.) were dissolved in i-PrOH (80.0 mL). The resulting mixture was stirred at 80° C. for 2 hr and tri-n-butyl phosphine (3.0 equiv.) was added. The reaction mixture was refluxed additionally for 16 hr. Then the reaction mixture was evaporated under reduced pressure and purified by flash chromatography to afford 6.2H.

### Step 1C: General Procedure for 6.2J

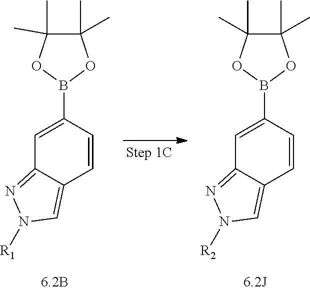

Potassium carbonate, anhydrous, 99% (3.0 equiv.) and iodomethane (1.7 equiv) were added to the solution of 6.2B (1.0 equiv.) in MeCN (appr. 45.0 mL). The resulting mixture was stirred at 25° C. for 16 hr. Then, solvent was removed under reduced pressure. The residue was diluted with water (40.0 mL) and the resulting mixture was extracted with DCM to 6.2J.

### Step 2: General Procedure for 6.2C

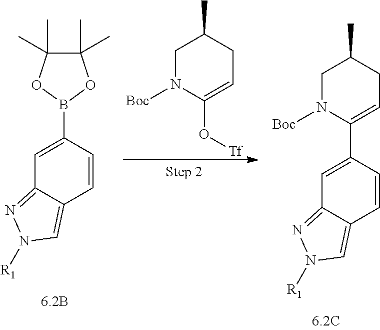

6.2B (1.0 equiv.), tert-butyl (3S)-3-methyl-6-(trifluoromethylsulfonyloxy)-3,4-dihydro-2H-pyridine-1-carboxylate (1.01 equiv.), sodium carbonate (3.0 equiv.) and Pd(dppf)Cl₂ DCM (0.05 equiv.) were stirred in a mixture of 1,4-dioxane (6.0 mL) and water (2.0 mL) under inert atmosphere at 85° C. for 15 hr. Upon completion, the reaction mixture was cooled down, diluted with water and extracted with DCM. The organic layer was separated, dried over Na₂SO₄ and concentrated under reduced pressure to afford 6.2C. The obtained material was used in the next step without an additional purification.

### Step 2A: General Procedure for 6.2B

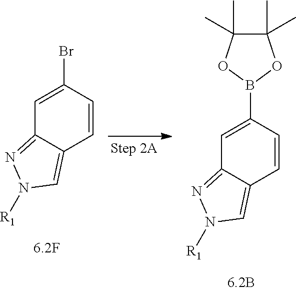

6.2F (1.0 equiv.), B₂Pin₂ (1.1 equiv.) and Potassium Acetate (2.0 equiv) were mixed together in 1.4-dioxane (appr. 20.0 mL). The resulting mixture was evacuated and then backfilled with argon, this operation was repeated three times, then Pd(dppf)₂Cl₂ DCM (993.01 mg, 1.22 mmol) was added under argon. The reaction mixture was stirred under argon atmosphere at 90° C. for 14 hr. Then the mixture was allowed to cool to the room temperature and the volatiles were removed in vacuo to afford 6.2B.

### Step 2B: General Procedure for 6.21

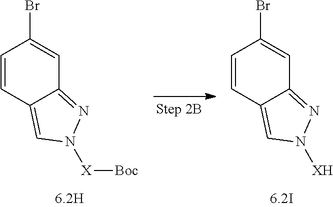

6.2H (1.0 equiv.) was dissolved in a mixture of TFA (1.0 mL) and DCM (1.0 mL). The reaction mixture was stirred at room temperature for 30 minutes. Then, the mixture was concentrated in vacuo to 6.21. The obtained material was used in the next step without an additional purification.

### Step 2C: General Procedure for 6.2C

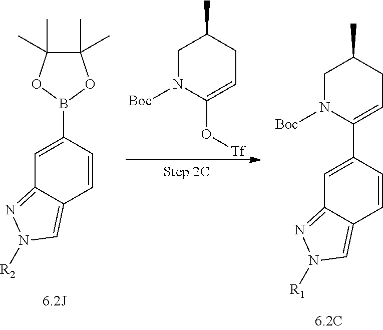

Sodium carbonate (2.0 equiv.) was added to a solution of 6.2J (1.0 equiv.) and tert-butyl (5S)-5-methyl-2-(trifluoromethylsulfonyloxy)piperidine-1-carboxylate (1.1 equiv.) in Water (appr. 15.0 mL) and 1,4-dioxane (appr. 50.0 mL). Reaction flask was evacuated and refilled with argon 3 times. Then, Pd(dppf)Cl₂·CH₂Cl₂ (0.05 equiv.) was added under stream of argon. The resulting mixture was stirred at 90° C. for 15 hr under inert atmosphere. Upon completion of the reaction, water (appr. 50.0 mL) was added. The resulting mixture was extracted with EtOAc (3*20.0 mL). The combined organic layers were dried over Na₂SO₄, filtered and concentrated in vacuo to obtain 6.2C. The obtained material was used in the next step without an additional purification.

### Step 3: General Procedure for 6.2D

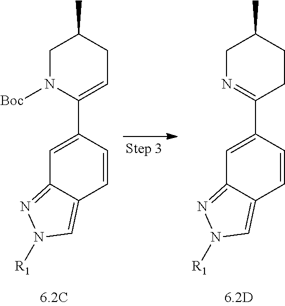

### Method A

6.2C (1.0 equiv.) was dissolved in a mixture of TFA (1.0 mL) and DCM (1.0 mL). The reaction mixture was stirred at room temperature for 30 minutes. Then, the mixture was concentrated in vacuo to 6.2D. The obtained material was used in the next step without an additional purification.

### Method B

6.2C (1.0 equiv.) was dissolved in a mixture of HCl (4.0M solution in dioxane, 1.0 mL) and MeOH (1.0 mL). The reaction mixture was stirred at room temperature overnight. Then, the mixture was concentrated in vacuo to 6.2D. The obtained material was used in the next step without an additional purification.

### Step 3B: General Procedure for 6.2F

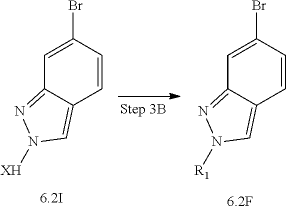

To a solution of 6.2I (1.0 equiv.), acetic acid (1.0 equiv.) and Formaldehyde, 37% in aq. soln., ACS, 36.5-38.0%, stab. with 10-15% methanol (1.0 equiv.) in DCM (appr. 25.0 mL), Sodium triacetoxyborohydride (1.0 equiv.) was added. The resulting mixture was stirred at 25° C. for 16 hr. Then, the solvent was removed in vacuo. The residue was poured in H₂O (50.0 mL) and extracted with EtOAc (2×20.0 mL). The combined organic extracts were washed with brine (2*20.0 mL), dried over sodium sulphate and evaporated in vacuo to afford 6.2F.

### Step 4: General Procedure for 6.2E

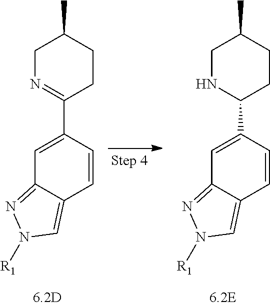

Sodium Borohydride (2.0 equiv) was portionwise added to the solution of 6.2D (1.0 equiv.) in MeOH (5.0 mL). The reaction mixture was stirred at room temperature for 17 hours. Then, the mixture was acidified with HCl (4.0M solution in dioxane) to pH 5 and the volatiles were removed in vacuo to afford 6.2E. The obtained material was used in the next step without an additional purification.

### Step 5: General Procedure for Product 6.2

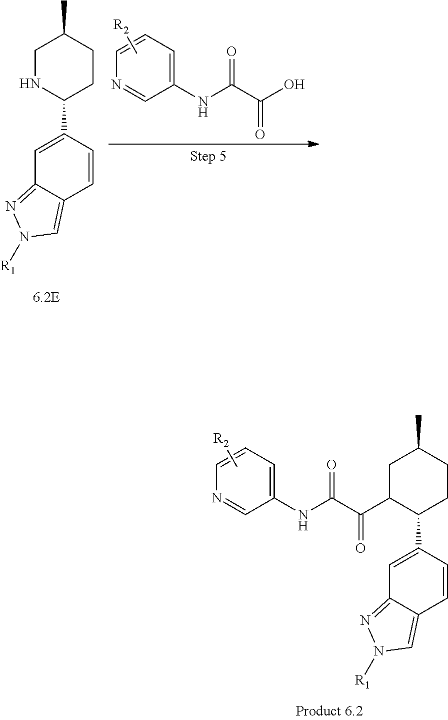

HATU (1.4 equiv.) was added to the stirred solution of 6.2E (1.0 equiv), corresponding acid (1.1 equiv) and DIPEA (10.0 equiv) in DMSO (appr. 6.0 mL). The resulting reaction mixture was stirred at 25° C. for 4 hr. The resulting mixture was submitted to reverse phase HPLC to afford Product 6.2.

### Example 65. Compound 117 N-(6-amino-5-ethylpyridin-3-yl)-2-((2R,5S)-5-methyl-2-(2-((S)-1-methylpyrrolidin-3-yl)-2H-indazol-6-yl)piperidin-1-yl)-2-oxoacetamide

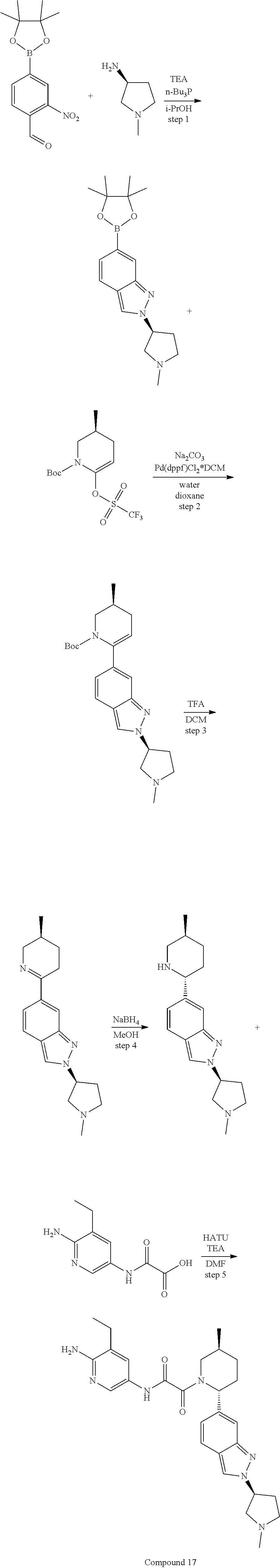

### Step 1: Synthesis of (S)-2-(1-methylpyrrolidin-3-yl)-6-(4,4,5,5-tetramethyl-1,3,2-dioxaborolan-2-yl)-2H-indazole

Prepared by general procedure scheme 6.2 step 1. Yield: 714 mg (40.31%).

FCC conditions: FCC (gradient MeOH in MTBE from 0% to 100%)

LCMS(ESI): [M]⁺ m/z: calcd 327.2; found 328.2; Rt=0.823 min.

### Step 2: Synthesis of (S)-tert-butyl 3-methyl-6-(2-((S)-1-methylpyrrolidin-3-yl)-2H-indazol-6-yl)-3,4-dihydropyridine-1(2H)-carboxylate

Prepared by general procedure scheme 6.2 step 2. Yield: 926 mg of crude.

LCMS(ESI): [M]⁺ m/z: calcd 396.2; found 397.2; Rt=1.207 min.

### Step 3: Synthesis of 6-((S)-5-methyl-3,4,5,6-tetrahydropyridin-2-yl)-2-((S)-1-methylpyrrolidin-3-yl)-2H-indazole

Prepared by general procedure scheme 6.2 step 3. Yield: 305 mg of crude.

LCMS(ESI): [M]⁺ m/z: calcd 296.2; found 297.2; Rt=0.524 min.

### Step 4: Synthesis of 6-((2R,5S)-5-methylpiperidin-2-yl)-2-((S)-1-methylpyrrolidin-3-yl)-2H-indazole

Prepared by general procedure scheme 6.2 step 3. Yield: 234 mg of crude.

LCMS(ESI): [M]⁺ m/z: calcd 298.2; found 299.2; Rt=0.433 min.

### Step 5: Synthesis of N-(6-amino-5-ethylpyridin-3-yl)-2-((2R,5S)-5-methyl-2-(2-((S)-1-methylpyrrolidin-3-yl)-2H-indazol-6-yl)piperidin-1-yl)-2-oxoacetamide (Compound 117)

Prepared by general procedure scheme 6.2 step 5. Yield: 22.7 mg (20.65%).

HPLC conditions: Column: SunFire C18 100*19 mm, 5 microM; 2-10 min 30-50% MeCN+NH₃ 30 ml/min; (loading pump 4 ml/min MeCN).

Compound 117: ¹H NMR (600 MHz, DMSO-d₆) δ (ppm) 0.73-1.05 (m, 3H), 1.07-1.16 (m, 3H), 1.28-1.43 (m, 1H), 1.63-1.79 (m, 1H), 1.81-1.93 (m, 1H), 2.02-2.24 (m, 2H), 2.31 (s, 3H), 2.33-2.37 (m, 1H), 2.38-2.46 (m, 3H), 2.52-2.58 (m, 2H), 2.79-2.87 (m, 2H), 2.91-2.97 (m, 1H), 3.47-4.04 (m, 1H), 5.14-5.61 (m, 2H), 5.61-5.70 (m, 2H), 6.92-7.09 (m, 1H), 7.43-7.55 (m, 2H), 7.63-7.74 (m, 1H), 7.95-8.11 (m, 1H), 8.32-8.45 (m, 1H), 10.45-10.64 (m, 1H).

LCMS(ESI): [M]⁺ m/z: calcd 489.2; found 490.2; Rt=1.805 min.

### Example 66. Compound 38 N-(6-amino-5-ethyl-3-pyridyl)-2-oxo-2-[(2R,5S)-2-[2-[2-(dimethylamino)ethyl]indazol-6-yl]-5-methyl-1-piperidyl]acetamide

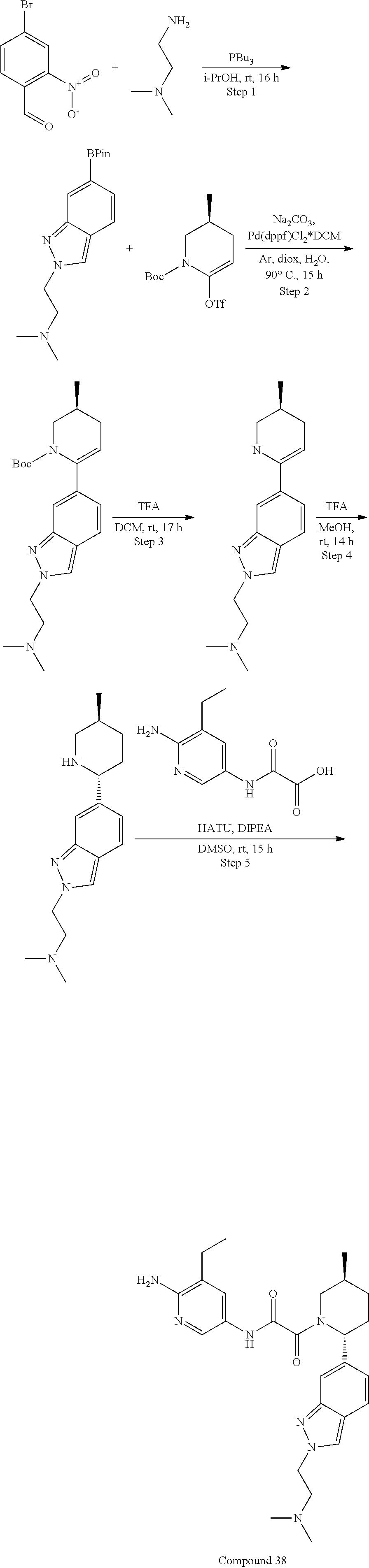

### Step 1: The Synthesis of N,N-dimethyl-2-[6-(4,4,5,5-tetramethyl-1,3,2-dioxaborolan-2-yl)indazol-2-yl]ethanamine

Prepared by general procedure Scheme 6.2 Step 1

The crude product was purified by FCC (MeOH in MTBE from 2% to 67%).

Yield: 1.45 g (50.98%)

LCMS(ESI): [M+H]⁺ m/z: calcd 315.2; found 316.2; Rt=1.056 min.

### Step 2: The Synthesis of tert-butyl (3S)-6-[2-[2-(dimethylamino)ethyl]indazol-6-yl]-3-methyl-3,4-dihydro-2H-pyridine-1-carboxylate

Prepared by general procedure Scheme 6.2 Step 2

Yield: 2.6 g (crude)

LCMS(ESI): [M+H]⁺ m/z: calcd 384.2; found 385.2; Rt=1.011 min.

### Step 3: The Synthesis of N,N-dimethyl-2-[6-[(3S)-3-methyl-2,3,4,5-tetrahydropyridin-6-yl]indazol-2-yl]ethanamine

Prepared by general procedure Scheme 6.2 Step 3 Yield: 0.9 g (46.8%)

LCMS(ESI): [M+H]⁺ m/z: calcd 284.2; found 285.2; Rt=0.407 min.

### Step 4: The Synthesis of N,N-dimethyl-2-[6-[(2R,5S)-5-methyl-2-piperidyl]indazol-2-yl]ethanamine

Prepared by general procedure Scheme 6.2 Step 4 (Method B) Yield: 0.7 g (77.2%)

LCMS(ESI): [M+H]⁺ m/z: calcd 286.2; found 287.2; Rt=0.414 min.

### Step 5: Synthesis of N-(6-amino-5-ethyl-3-pyridyl)-2-oxo-2-[(2R,5S)-2-[2-[2-(dimethylamino)ethyl]indazol-6-yl]-5-methyl-1-piperidyl]acetamide (Compound 38)

Prepared by general procedure Scheme 6.2 Step 5

HPLC conditions: 2-10 min 10-50% methanol+NH3, 30 ml/min (loading pump 4 ml methanol+NH3) column: YMC-ACTUS TRIART C18 100*20 5 microM

Yield: 38 mg (79.56%)

¹H NMR (600 MHz, dmso) δ 0.74-1.05 (m, 3H), 1.06-1.15 (m, 3H), 1.30-1.42 (m, 1H), 1.58-1.79 (m, 1H), 1.81-1.95 (m, 1H), 2.01-2.15 (m, 1H), 2.17 (s, 6H), 2.24-2.33 (m, 1H), 2.35-2.43 (m, 2H), 2.73-2.79 (m, 2H), 2.79-3.23 (m, 1H), 3.46-4.07 (m, 1H), 4.46-4.51 (m, 2H), 5.17-5.61 (m, 1H), 5.62-5.71 (m, 2H), 6.94-7.08 (m, 1H), 7.44-7.54 (m, 2H), 7.64-7.71 (m, 1H), 7.99-8.10 (m, 1H), 8.30-8.37 (m, 1H), 10.43-10.65 (m, 1H).

LCMS(ESI): [M+H]⁺ m/z: calcd 477.3; found 478.2; Rt=1.646 min.

### Example 67. Compound 85 and Compound 60 N-(6-amino-5-ethylpyridin-3-yl)-2-((2R,5S)-2-(2-(1,5-dimethylpiperidin-3-yl)-2H-indazol-6-yl)-5-methylpiperidin-1-yl)-2-oxoacetamide

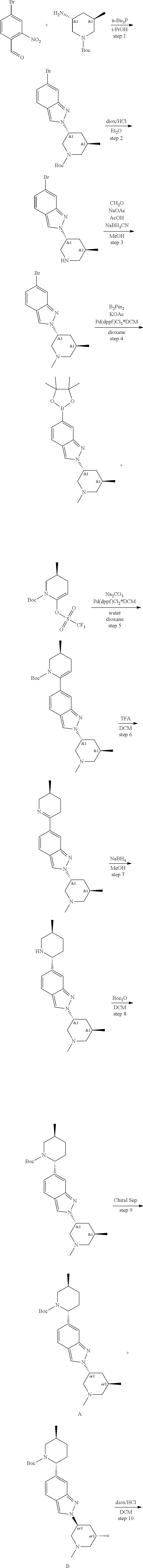

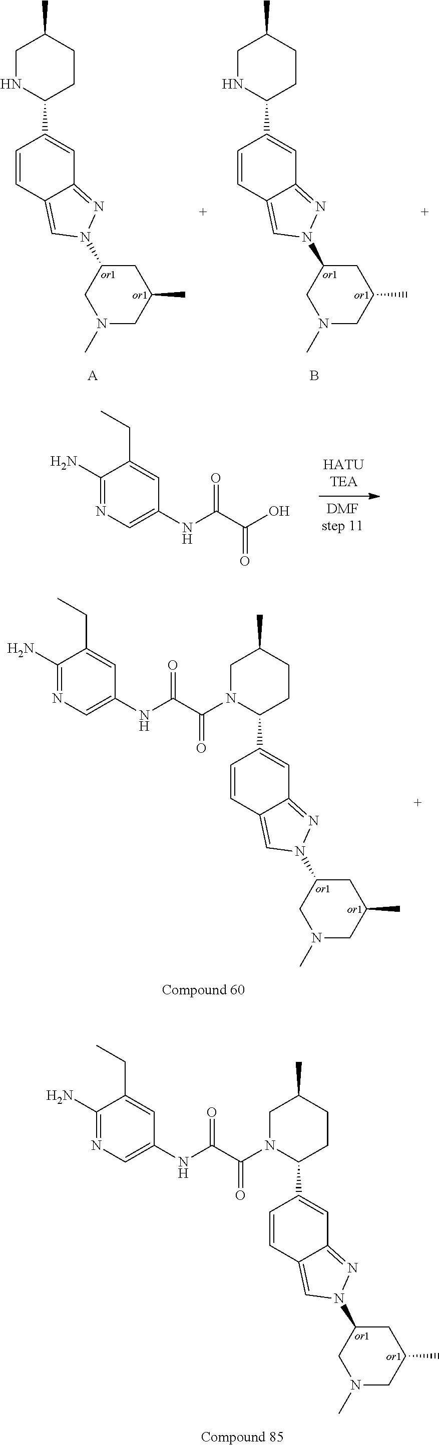

### Step 1: Synthesis of rac-(3R,5R)-tert-butyl 3-(6-bromo-2H-indazol-2-yl)-5-methylpiperidine-1-carboxylate

Prepared by general procedure scheme 6.2 step 1A. Yield: 5.8 g (70.05%).

FCC conditions: FCC (SiO₂; Hexane/MTBE with MTBE from 0 to 40%)

LCMS(ESI): [M]⁺ m/z: calcd 394.2; found 395.2; Rt=1.406 min.

### Step 2: Synthesis of rac-6-bromo-2-((3R,5R)-5-methylpiperidin-3-yl)-2H-indazole

To a solution of tert-butyl rac-(3R,5R)-3-(6-bromoindazol-2-yl)-5-methyl-piperidine-1-carboxylate (4.66 g, 11.82 mmol) in Et₂O (50 mL) was added hydrogen chloride solution 4.0M in dioxane (32.00 g, 877.65 mmol, 40 mL) at 21° C. The resulting mixture was left to stir for 18 hr. The reaction mixture was evaporated to dryness to give 6-bromo-2-[rac-(3R,5R)-5-methyl-3-piperidyl]indazole (5.25 g, crude, 2HCl) as a light-yellow solid.

¹H NMR (500 MHz, DMSO-d₆) δ 0.95 (d, 3H), 1.89 (m, 1H), 1.97 (m, 1H), 2.32 (m, 1H), 2.75 (m, 1H), 3.15 (m, 2H), 3.44 (m, 1H), 7.17 (d, 1H), 7.72 (d, 1H), 7.85 (s, 1H), 8.53 (s, 1H), 8.65 (s, 1H), 9.98 (s, 1H).

### Step 3: Synthesis of rac-6-bromo-2-((3R,5R)-1,5-dimethylpiperidin-3-yl)-2H-indazole

Formaldehyde, 37% w/w aq. soln., stab. with 7-8% MeOH (1.02 g, 12.58 mmol, 943.12 μL, 37% purity) and acetic acid (2.06 g, 34.32 mmol, 1.96 mL) were added to the solution of 6-bromo-2-[rac-(3R,5R)-5-methyl-3-piperidyl]indazole (5.25 g, 11.44 mmol, 2HCl) and sodium acetate, anhydrous (1.97 g, 24.03 mmol, 1.29 mL) in MeOH. Resulting mixture was stirred at 21° C. for 1 hr before sodium cyan borohydride (1.44 g, 22.88 mmol) was added thereto. After that, stirring was continued for 21 hr. Then, the solvent was removed under reduced pressure, and residue was partitioned between 10% aq. NaOH solution (20 ml) and DCM (40 ml). The organic layer was separated, evaporated. 6-Bromo-2-[rac-(3R,5R)-1,5-dimethyl-3-piperidyl]indazole (3.35 g, 10.87 mmol, 95.00% yield) was obtained as a beige solid.

LCMS(ESI): [M]⁺ m/z: calcd 308.2; found 309.2; Rt=2.300 min.

### Step 4: Synthesis of rac-2-((3R,5R)-1,5-dimethylpiperidin-3-yl)-6-(4,4,5,5-tetramethyl-1,3,2-dioxaborolan-2-yl)-2H-indazole

Prepared by general procedure scheme 6.2 step 2A. Yield: 5 g of crude.

LCMS(ESI): [M]⁺ m/z: calcd 355.2; found 356.2; Rt=2.665 min.

### Step 5: Synthesis of (S)-tert-butyl 6-(2-(rac-(3R,5R)-1,5-dimethylpiperidin-3-yl)-2H-indazol-6-yl)-3-methyl-3,4-dihydropyridine-1(2H)-carboxylate

Prepared by general procedure scheme 6.2 step 2. Yield: 8 g of crude.

LCMS(ESI): [M]⁺ m/z: calcd 424.2; found 425.2; Rt=3.258 min.

### Step 6: Synthesis of 2-(rac-(3R,5R)-1,5-dimethylpiperidin-3-yl)-6-((S)-5-methyl-3,4,5,6-tetrahydropyridin-2-yl)-2H-indazole

Prepared by general procedure scheme 6.2 step 3. Yield: 7.3 g of crude.

LCMS(ESI): [M]⁺ m/z: calcd 324.2; found 325.2; Rt=1.158 min.

### Step 7: Synthesis of 2-(rac-(3R,5R)-1,5-dimethylpiperidin-3-yl)-6-((2R,5S)-5-methylpiperidin-2-yl)-2H-indazole

Prepared by general procedure scheme 6.2 step 4. Yield: 0.57 g (11.33%).

FCC conditions: FCC (SiO₂; CHCl₃ with MTBE from 0 to 30%)

LCMS(ESI): [M]⁺ m/z: calcd 326.2; found 327.2; Rt=1.095 min.

### Step 8: Synthesis of (2R,5S)-tert-butyl 2-(2-(rac-(3R,5R)-1,5-dimethylpiperidin-3-yl)-2H-indazol-6-yl)-5-methylpiperidine-1-carboxylate

To a solution of 2-(rac-(3R,5R)-1,5-dimethylpiperidin-3-yl)-6-((2R,5S)-5-methylpiperidin-2-yl)-2H-indazole (0.57 g, 1.75 mmol) in DCM (25 mL) was added di-tert-butyl dicarbonate (381.04 mg, 1.75 mmol, 400.67 μL) portionwise at 21° C. The resulting solution was washed with 10% aq. HCl and brine, dried over Na₂SO₄ and evaporated to dryness and purified by HPLC (70-100% 0.5-6.5 min water-MeOH; flow 30 ml/min (loading pump 4 ml/min MeCN); column SUNFIRE 100×19 mm 5 um (R)) to give (2R,5S)-tert-butyl 2-(2-(rac-(3R,5R)-1,5-dimethylpiperidin-3-yl)-2H-indazol-6-yl)-5-methylpiperidine-1-carboxylate (246.3 mg, 577.36 mol, 33.07% yield) as a brown solid.

LCMS(ESI): [M]⁺ m/z: calcd 426.2; found 427.2; Rt=3.109 min.

### Step 9: Chiral Separation

Racemic (2R,5S)-tert-butyl 2-(2-(rac-(3R,5R)-1,5-dimethylpiperidin-3-yl)-2H-indazol-6-yl)-5-methylpiperidine-1-carboxylate (246.3 mg, 577.36 mol) was chiral separated (Column: Chiralpak IA III (250*20 mm, 5 mkm); Mobile phase: Hexane-IPA-MeOH, 90-5-5, 15 ml/min) to obtain tert-butyl (2R,5S)-5-methyl-2-[2-[(3S,5S)-1,5-dimethyl-3-piperidyl]indazol-6-yl]piperidine-1-carboxylate (60.94 mg, 142.85 mol, 24.74% yield) (RT=13.29 min) and tert-butyl (2R,5S)-5-methyl-2-[2-[(3R,5R)-1,5-dimethyl-3-piperidyl]indazol-6-yl]piperidine-1-carboxylate (69.92 mg, 163.90 mol, 28.39% yield) (RT=16.43 min).

Rel Time for A in analytical conditions (column: IA, Hexane-IPA-MeOH, 90-5-5, 1 ml/min as mobile phase) 14.58 min and for B 11.25 min.

### A: Retention Time: 14.58 Min

LCMS(ESI): [M]⁺ m/z: calcd 426.2; found 427.2; Rt=1.124 min.

### B: Retention Time: 11.25 Min

LCMS(ESI): [M]⁺ m/z: calcd 426.2; found 427.2; Rt=1.148 min.

### Step 10: Synthesis of 2-(1,5-dimethylpiperidin-3-yl)-6-((2R,5S)-5-methylpiperidin-2-yl)-2H-indazole

A: To a solution of tert-butyl (2R,5S)-5-methyl-2-[2-[(3R,5R)-1,5-dimethyl-3-piperidyl]indazol-6-yl]piperidine-1-carboxylate (69.92 mg, 163.90 mol) in dioxane (5 mL) was added hydrogen chloride solution 4.0M in dioxane (5.98 mg, 163.90 mol, 7.47 μL) at 21° C. The resulting mixture was left to stir for 2 hr. The resulting mixture was evaporated to dryness to give 2-[(3R,5R)-1,5-dimethyl-3-piperidyl]-6-[(2R,5S)-5-methyl-2-piperidyl]indazole (70 mg, 160.60 mol, 97.99% yield, 3HCl) as a yellow solid.

LCMS(ESI): [M]⁺ m/z: calcd 326.2; found 327.2; Rt=1.601 min.

B: To a solution of tert-butyl (2R,5S)-5-methyl-2-[2-[(3S,5S)-1,5-dimethyl-3-piperidyl]indazol-6-yl]piperidine-1-carboxylate (60.94 mg, 142.85 mol) in DCM (2 mL) was added hydrogen chloride solution 4.0M in dioxane (5.21 mg, 142.85 mol, 6.51 μL) at 21° C. The resulting mixture was left to stir for 2 hr. The resulting mixture was evaporated to dryness. 2-[(3S,5S)-1,5-Dimethyl-3-piperidyl]-6-[(2R,5S)-5-methyl-2-piperidyl]indazole (60 mg, 137.66 mol, 96.36% yield, 3HCl) was obtained as a beige solid.

LCMS(ESI): [M]⁺ m/z: calcd 326.2; found 327.2; Rt=1.609 min.

### Step 11: Synthesis of N-(6-amino-5-ethylpyridin-3-yl)-2-((2R,5S)-2-(2-(1,5-dimethylpiperidin-3-yl)-2H-indazol-6-yl)-5-methylpiperidin-1-yl)-2-oxoacetamide (Compound 85 and Compound 60)

Prepared by general procedure scheme 6.2 step 5. Yield: 49.8 mg (69.88%).

HPLC conditions: Column: SunFire C18 100*19 mm, 5 microM; 0.5-6.5 min 40-60% water-MeCN+NH₃30 ml/min; (loading pump 4 ml/min MeCN).

Compound 85: ¹H NMR (600 MHz, DMSO-d₆) δ (ppm) 1.03 (m, 9H), 1.36 (m, 1H), 1.57 (m, 1H), 1.82 (m, 4H), 2.22 (s, 5H), 2.38 (m, 3H), 2.79 (m, 4H), 3.65 (m, 1H), 5.33 (m, 4H), 7.00 (m, 1H), 7.48 (m, 2H), 7.69 (m, 1H), 8.03 (m, 1H), 8.57 (s, 1H), 10.53 (m, 1H).

LCMS(ESI): [M]⁺ m/z: calcd 517.2; found 518.2; Rt=2.170 min.

Prepared by general procedure scheme 6.2 step 5. Yield: 49.8 mg (69.88%).

HPLC conditions: Column: SunFire C18 100*19 mm, 5 microM; 0.5-6.5 min 40-60% water-MeCN+NH₃30 ml/min; (loading pump 4 ml/min MeCN).

Compound 60: ¹H NMR (600 MHz, DMSO-d₆) δ (ppm) 0.90 (m, 3H), 1.02 (m, 3H), 1.11 (m, 3H), 1.36 (m, 1H), 1.56 (m, 1H), 1.76 (m, 2H), 1.88 (m, 2H), 2.06 (m, 1H), 2.22 (s, 5H), 2.38 (m, 3H), 2.58 (m, 2H), 2.87 (m, 1H), 3.85 (m, 1H), 5.33 (m, 4H), 6.99 (m, 1H), 7.49 (m, 2H), 7.70 (m, 1H), 8.04 (m, 1H), 8.58 (s, 1H), 10.53 (m, 1H).

LCMS(ESI): [M]⁺ m/z: calcd 517.2; found 518.2; Rt=2.210 min.

### Example 68. Compound 115 and Compound 48 N-(6-amino-5-ethylpyridin-3-yl)-2-((2R,5S)-5-methyl-2-(2-(1,2,2-trimethylpiperidin-4-yl)-2H-indazol-6-yl)piperidin-1-yl)-2-oxoacetamide

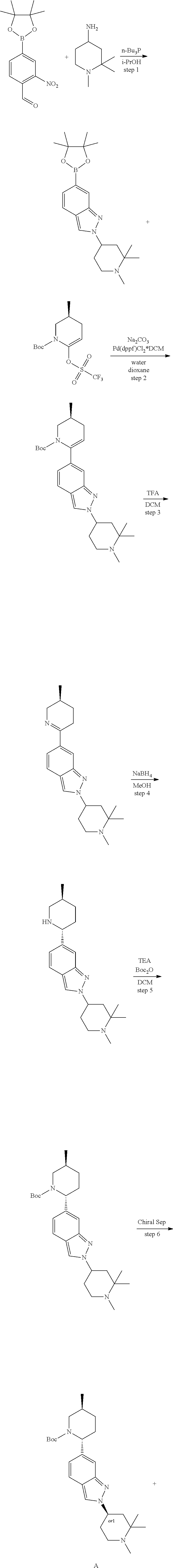

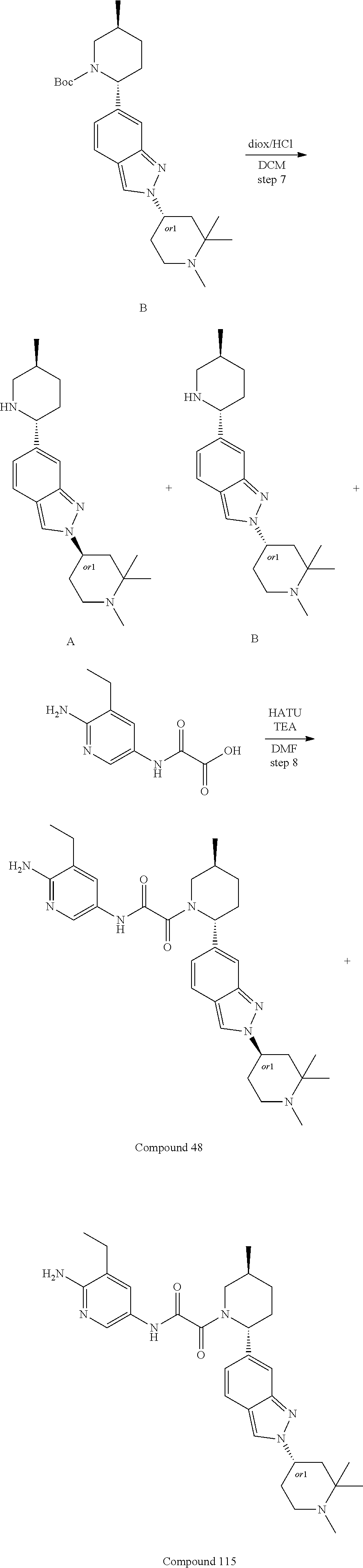

The synthesis of 2-nitro-4-(4,4,5,5-tetramethyl-1,3,2-dioxaborolan-2-yl)benzaldehyde is given by the following. 4-Bromo-2-nitro-benzaldehyde (25.3 g, 109.99 mmol), 4,4,5,5-tetramethyl-2-(4,4,5,5-tetramethyl-1,3,2-dioxaborolan-2-yl)-1,3,2-dioxaborolane (29.33 g, 115.49 mmol) and potassium acetate (26.99 g, 274.98 mmol, 17.19 mL) were mixed together in dioxane (350 mL). The resulting mixture was evacuated and backfilled three times with argon and Pd(dppf)Cl₂*DCM (4.49 g, 5.50 mmol) was added thereto. The resulting mixture was heated at 100° C. overnight. The reaction mixture was concentrated in vacuum and water (250 ml) was added thereto. The resulting mixture was extracted with EtOAc (2*250 ml) and combined organic layers were washed with brine, dried over Na₂SO₄, filtered and concentrated in vacuum. The residue was triturated with hexane, insoluble impurities were filtered and the filtrate was concentrated in vacuum to obtain 2-nitro-4-(4,4,5,5-tetramethyl-1,3,2-dioxaborolan-2-yl)benzaldehyde (29.56 g, crude).

LCMS(ESI): [M]f m/z: calcd 277.2; found 278.2; Rt=0.725 min.

### Step 1: Synthesis of 6-(4,4,5,5-tetramethyl-1,3,2-dioxaborolan-2-yl)-2-(1,2,2-trimethylpiperidin-4-yl)-2H-indazole

Prepared by general procedure scheme 6.2 step 1A. Yield: 4.29 g (45.84%).

FCC conditions: FCC (SiO₂; MeOH in MTBE from 0 to 100%)

LCMS(ESI): [M]⁺ m/z: calcd 369.2; found 370.2; Rt=1.167 min.

### Step 2: Synthesis of (3S)-tert-butyl 3-methyl-6-(2-(1,2,2-trimethylpiperidin-4-yl)-2H-indazol-6-yl)-3,4-dihydropyridine-1(2H)-carboxylate

Prepared by general procedure scheme 6.2 step 2. Yield: 4.72 g of crude.

LCMS(ESI): [M]⁺ m/z: calcd 438.2; found 439.2; Rt=1.097 min.

### Step 3: Synthesis of 6-((S)-5-methyl-3,4,5,6-tetrahydropyridin-2-yl)-2-(1,2,2-trimethylpiperidin-4-yl)-2H-indazole

Prepared by general procedure scheme 6.2 step 3. Yield: 4.14 g of crude.

LCMS(ESI): [M]⁺ m/z: calcd 338.2; found 339.2; Rt=0.663 min.

### Step 4: Synthesis of 6-((2R,5S)-5-methylpiperidin-2-yl)-2-(1,2,2-trimethylpiperidin-4-yl)-2H-indazole

Prepared by general procedure scheme 6.2 step 4. Yield: 4.25 g of crude.

LCMS(ESI): [M]⁺ m/z: calcd 340.2; found 341.2; Rt=0.735 min.

### Step 5: Synthesis of (2R,5S)-tert-butyl 5-methyl-2-(2-(1,2,2-trimethylpiperidin-4-yl)-2H-indazol-6-yl)piperidine-1-carboxylate

6-[(2R,5S)-5-Methyl-2-piperidyl]-2-(1,2,2-trimethyl-4-piperidyl)indazole (4.25 g, 12.48 mmol) was dissolved in DCM (75 mL) and TEA (1.89 g, 18.72 mmol, 2.61 mL) was added thereto. Di-tert-butyl dicarbonate (3.27 g, 14.98 mmol, 3.44 mL) was added to the previous solution and the resulting mixture was stirred overnight. The reaction mixture was concentrated in vacuum and the residue was purified by column chromatography (normal phase: gradient MeOH in MTBE from 0% to 100%; reversed-phase: gradient MeOH in water from 0% to 100%) to obtain tert-butyl (2R,5S)-5-methyl-2-[2-(1,2,2-trimethyl-4-piperidyl)indazol-6-yl]piperidine-1-carboxylate (100 mg, 226.95 mol, 1.82% yield), tert-butyl (2R,5S)-5-methyl-2-[2-(1,2,2-trimethyl-4-piperidyl)indazol-6-yl]piperidine-1-carboxylate (1 g, 2.27 mmol, 18.18% yield) and tert-butyl (2R,5S)-5-methyl-2-[2-(1,2,2-trimethyl-4-piperidyl)indazol-6-yl]piperidine-1-carboxylate (365 mg, 828.38 μmol, 6.64% yield).

LCMS(ESI): [M]⁺ m/z: calcd 440.2; found 441.2; Rt=1.343 min.

### Step 6: Chiral Separation)

Racemic tert-butyl (2R,5S)-5-methyl-2-[2-(1,2,2-trimethyl-4-piperidyl)indazol-6-yl]piperidine-1-carboxylate (1 g, 2.27 mmol) was chiral separated (Column: CHIRALCEL OJ-H 250*20, 5-I Mobile phase: Hexane-IPA-MeOH, 95-2.5-2.5 Flow rate: 14 ml/min) to obtain tert-butyl (2R,5S)-5-methyl-2-[2-[(45)-1,2,2-trimethyl-4-piperidyl]indazol-6-yl]piperidine-1-carboxylate (183.74 mg, 417.00 mol, 18.37% yield) (RT=11.75 min) and tert-butyl (2R,5S)-5-methyl-2-[2-[(4R)-1,2,2-trimethyl-4-piperidyl]indazol-6-yl]piperidine-1-carboxylate (208.13 mg, 472.36 μmol, 20.81% yield) (RT=14.61 min).

Rel Time for A in analytical conditions (column: OJ-H, Hexane-IPA-MeOH, 90-5-5, 0.6 ml/min as mobile phase) 10.44 min and for B 8.31 min.

### A: Retention Time: 10.44 Min

LCMS(ESI): [M]⁺ m/z: calcd 440.2; found 441.2; Rt=3.321 min.

### B: Retention Time: 8.31 Min

LCMS(ESI): [M]⁺ m/z: calcd 440.2; found 441.2; Rt=3.310 min.

### Step 7: Synthesis of 6-((2R,5S)-5-methylpiperidin-2-yl)-2-(1,2,2-trimethylpiperidin-4-yl)-2H-indazole

A: tert-Butyl (2R,5S)-5-methyl-2-[2-[(4R)-1,2,2-trimethyl-4-piperidyl]indazol-6-yl]piperidine-1-carboxylate (208.13 mg, 472.36 mol) was dissolved in MeOH (4 mL) and HCl/dioxane (4 mL) was added thereto. The resulting mixture was stirred for 4 hr. The reaction mixture was concentrated in vacuum to obtain 6-[(2R,5S)-5-methyl-2-piperidyl]-2-[(4R)-1,2,2-trimethyl-4-piperidyl]indazole (196 mg, crude, 2HCl).

LCMS(ESI): [M]⁺ m/z: calcd 340.2; found 341.2; Rt=0.742 min.

B: tert-Butyl (2R,5S)-5-methyl-2-[2-[(4S)-1,2,2-trimethyl-4-piperidyl]indazol-6-yl]piperidine-1-carboxylate (183.74 mg, 417.00 mol) was dissolved in MeOH (4 mL) and HCl/dioxane (4 mL) was added thereto. The resulting mixture was stirred for 4 hr. The reaction mixture was concentrated in vacuum to obtain 6-[(2R,5S)-5-methyl-2-piperidyl]-2-[(45)-1,2,2-trimethyl-4-piperidyl]indazole (189 mg, crude, 2HCl).

LCMS(ESI): [M]⁺ m/z: calcd 340.2; found 341.2; Rt=0.738 min.

### Step 8: Synthesis of N-(6-amino-5-ethylpyridin-3-yl)-2-((2R,5S)-5-methyl-2-(2-(1,2,2-trimethylpiperidin-4-yl)-2H-indazol-6-yl)piperidin-1-yl)-2-oxoacetamide (Compound 48 and Compound 115)

Prepared by general procedure scheme 6.2 step 5. Yield: 109.4 mg (38.48%).

HPLC conditions: Column: SunFire C18 100*19 mm, 5 microM; 2-10 min 25-50% MeOH+NH₃30 ml/min; (loading pump 4 ml/min MeOH).

Compound 48: ¹H NMR (600 MHz, DMSO-d₆) δ (ppm) 1.00-1.15 (m, 12H), 1.30-1.40 (m, 1H), 1.75-1.96 (m, 4H), 1.98-2.11 (m, 3H), 2.14-2.36 (m, 5H), 2.41 (q, 1H), 2.56 (t, 1H), 2.66 (d, 1H), 2.75-3.25 (m, 1H), 3.46-4.05 (m, 1H), 4.63-4.70 (m, 1H), 5.15-5.71 (m, 3H), 7.12-7.27 (m, 1H), 7.41-7.55 (m, 1H), 7.55-7.65 (m, 2H), 7.98-8.11 (m, 1H), 8.34-8.43 (m, 1H), 10.51 (s, 1H).

LCMS(ESI): [M]⁺ m/z: calcd 531.2; found 532.2; Rt=2.155 min.

Prepared by general procedure scheme 6.2 step 5. Yield: 123.5 mg (50.81%).

HPLC conditions: Column: SunFire C18 100*19 mm, 5 microM; 2-10 min 25-50% water-MeOH+NH₃30 ml/min; (loading pump 4 ml/min MeOH).

Compound 115: ¹H NMR (600 MHz, DMSO-d₆) δ (ppm) 0.97-1.17 (m, 12H), 1.30-1.40 (m, 1H), 1.72-2.11 (m, 7H), 2.12-2.36 (m, 5H), 2.40 (q, 1H), 2.56 (t, 1H), 2.66 (d, 1H), 2.76-3.26 (m, 1H), 3.44-4.04 (m, 1H), 4.61-4.71 (m, 1H), 5.11-5.69 (m, 3H), 7.09-7.28 (m, 1H), 7.42-7.55 (m, 1H), 7.55-7.78 (m, 2H), 7.95-8.10 (m, 1H), 8.34-8.44 (m, 1H), 10.51 (s, 1H).

LCMS(ESI): [M]⁺ m/z: calcd 531.2; found 532.2; Rt=2.145 min.

### Example 69. Compound 112 N-(6-amino-5-ethylpyridin-3-yl)-2-((2R,5S)-5-methyl-2-(2-((R)-1-methylpyrrolidin-3-yl)-2H-indazol-6-yl)piperidin-1-yl)-2-oxoacetamide

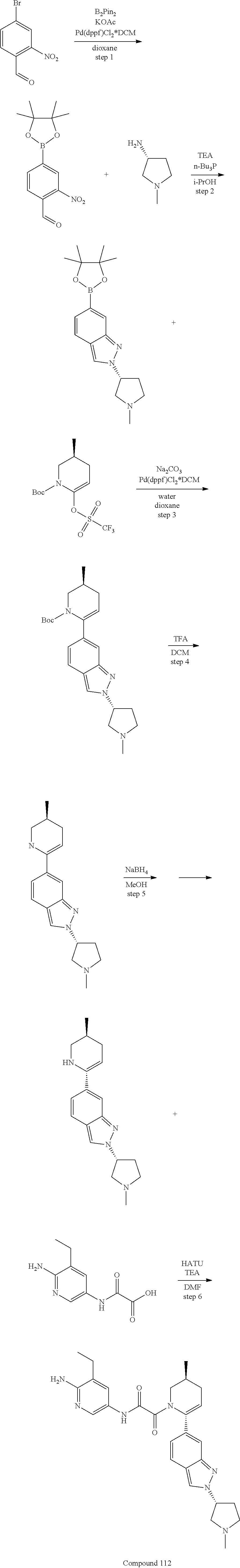

### Step 1: Synthesis of 2-nitro-4-(4,4,5,5-tetramethyl-1,3,2-dioxaborolan-2-yl)benzaldehyde

4-Bromo-2-nitro-benzaldehyde (25.3 g, 109.99 mmol), 4,4,5,5-tetramethyl-2-(4,4,5,5-tetramethyl-1,3,2-dioxaborolan-2-yl)-1,3,2-dioxaborolane (29.33 g, 115.49 mmol) and potassium acetate (26.99 g, 274.98 mmol, 17.19 mL) were mixed together in dioxane (350 mL). The resulting mixture was evacuated and backfilled three times with argon and Pd(dppf)Cl₂*DCM (4.49 g, 5.50 mmol) was added thereto. The resulting mixture was heated at 100° C. overnight. The reaction mixture was concentrated in vacuum and water (250 ml) was added thereto. The resulting mixture was extracted with EtOAc (2*250 ml) and combined organic layers were washed with brine, dried over Na₂SO₄, filtered and concentrated in vacuum. The residue was triturated with hexane, insoluble impurities were filtered and the filtrate was concentrated in vacuum to obtain 2-nitro-4-(4,4,5,5-tetramethyl-1,3,2-dioxaborolan-2-yl)benzaldehyde (29.56 g, crude).

LCMS(ESI): [M]⁺ m/z: calcd 277.2; found 278.2; Rt=0.725 min.

### Step 2: Synthesis of (R)-2-(1-methylpyrrolidin-3-yl)-6-(4,4,5,5-tetramethyl-1,3,2-dioxaborolan-2-yl)-2H-indazole

Prepared by general procedure scheme 6.2 step 1. Yield: 1.8 g (12.26%).

FCC conditions: FCC (gradient MeOH in MTBE from 0% to 50%)

LCMS(ESI): [M]⁺ m/z: calcd 327.2; found 328.2; Rt=1.028 min.

### Step 3: Synthesis of (S)-tert-butyl 3-methyl-6-(2-((R)-1-methylpyrrolidin-3-yl)-2H-indazol-6-yl)-3,4-dihydropyridine-1(2H)-carboxylate

Prepared by general procedure scheme 6.2 step 2. Yield: 1.11 g of crude.

LCMS(ESI): [M]⁺ m/z: calcd 396.2; found 397.2; Rt=1.070 min.

### Step 4: Synthesis of 6-((S)-5-methyl-3,4,5,6-tetrahydropyridin-2-yl)-2-((R)-1-methylpyrrolidin-3-yl)-2H-indazole

Prepared by general procedure scheme 6.2 step 3. Yield: 484 mg of crude.

LCMS(ESI): [M]⁺ m/z: calcd 296.2; found 297.2; Rt=0.433 min.

### Step 5: Synthesis of 6-((2R,5S)-5-methylpiperidin-2-yl)-2-((R)-1-methylpyrrolidin-3-yl)-2H-indazole

Prepared by general procedure scheme 6.2 step 3. Yield: 510 mg of crude.

LCMS(ESI): [M]⁺ m/z: calcd 298.2; found 299.2; Rt=0.622 min.

### Step 6: Synthesis of N-(6-amino-5-ethylpyridin-3-yl)-2-((2R,5S)-5-methyl-2-(2-((R)-1-methylpyrrolidin-3-yl)-2H-indazol-6-yl)piperidin-1-yl)-2-oxoacetamide (Compound 112)

Prepared by general procedure scheme 6.2 step 5. Yield: 54.4 mg (22.11%).

HPLC conditions: Column: SunFire C18 100*19 mm, 5 microM; 2-10 min 30-60% MeOH+NH₃30 ml/min; (loading pump 4 ml/min MeOH).

Compound 112: ¹H NMR (600 MHz, DMSO-d₆) δ (ppm) 0.99-1.17 (m, 6H), 1.30-1.42 (m, 1H), 1.68-1.80 (m, 1H), 1.83-1.93 (m, 1H), 2.01-2.28 (m, 3H), 2.31 (s, 3H), 2.32-2.36 (m, 1H), 2.38-2.44 (m, 2H), 2.53-2.58 (m, 1H), 2.59-2.99 (m, 4H), 3.46-4.08 (m, 1H), 5.14-5.69 (m, 4H), 6.92-7.10 (m, 1H), 7.40-7.58 (m, 2H), 7.64-7.72 (m, 1H), 7.99-8.10 (m, 1H), 8.39 (s, 1H), 10.46-10.62 (m, 1H).

LCMS(ESI): [M]⁺ m/z: calcd 489.2; found 490.2; Rt=1.711 min.

### Example 70. Compound 114 and Compound 78 N-(6-amino-5-ethylpyridin-3-yl)-2-((2R,5S)-5-methyl-2-(2-(1,5,5-trimethylpiperidin-3-yl)-2H-indazol-6-yl)piperidin-1-yl)-2-oxoacetamide

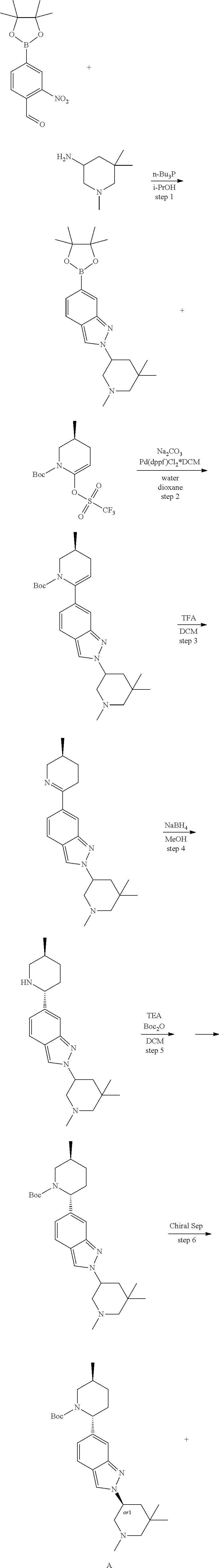

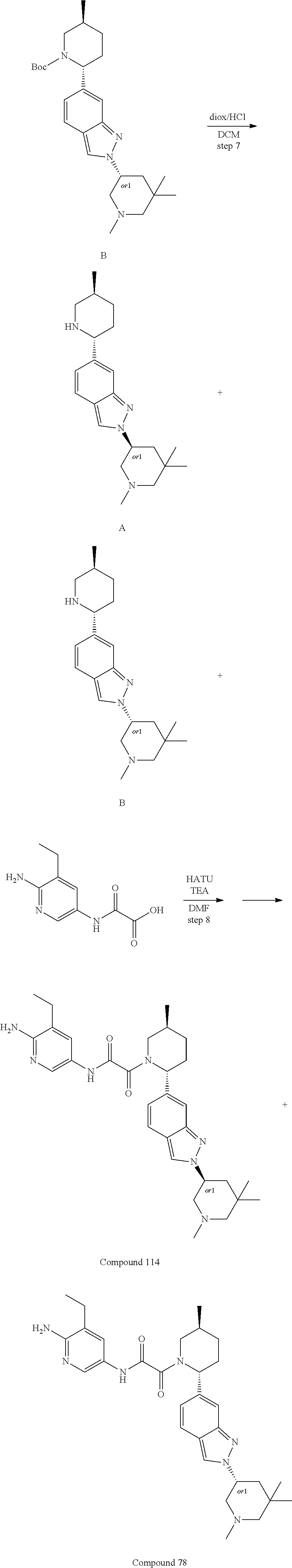

### Step 1: Synthesis of 6-(4,4,5,5-tetramethyl-1,3,2-dioxaborolan-2-yl)-2-(1,5,5-trimethylpiperidin-3-yl)-2H-indazole

Prepared by general procedure scheme 6.2 step 1A. Yield: 5.61 g (49.89%).

FCC conditions: FCC (SiO₂; MTBE in Hexane from 0 to 100%)

LCMS(ESI): [M]⁺ m/z: calcd 369.2; found 370.2; Rt=1.191 min.

### Step 2: Synthesis of (3S)-tert-butyl 3-methyl-6-(2-(1,5,5-trimethylpiperidin-3-yl)-2H-indazol-6-yl)-3,4-dihydropyridine-1(2H)-carboxylate

Prepared by general procedure scheme 6.2 step 2. Yield: 7.68 g of crude.

LCMS(ESI): [M]⁺ m/z: calcd 438.2; found 439.2; Rt=1.248 min.

### Step 3: Synthesis of 6-((S)-5-methyl-3,4,5,6-tetrahydropyridin-2-yl)-2-(1,5,5-trimethylpiperidin-3-yl)-2H-indazole

Prepared by general procedure scheme 6.2 step 3. Yield: 6.27 g of crude.

¹H NMR (400 MHz, CDCl₃) δ 1.01 (s, 6H), 1.24 (d, 3H), 1.34 (m, 1H), 1.88 (m, 8H), 2.33 (s, 3H), 2.85 (m, 1H), 3.27 (m, 2H), 4.04 (m, 1H), 4.77 (m, 1H), 7.60 (m, 1H), 7.77 (m, 1H), 7.91 (m, 1H), 7.99 (m, 1H).

### Step 4: Synthesis of 6-((2R,5S)-5-methylpiperidin-2-yl)-2-(1,5,5-trimethylpiperidin-3-yl)-2H-indazole

Prepared by general procedure scheme 6.2 step 4. Yield: 5.18 g of crude.

LCMS(ESI): [M]⁺ m/z: calcd 340.2; found 341.2; Rt=0.746 min.

### Step 5: Synthesis of (2R,5S)-tert-butyl 5-methyl-2-(2-(1,5,5-trimethylpiperidin-3-yl)-2H-indazol-6-yl)piperidine-1-carboxylate

6-[(2R,5S)-5-Methyl-2-piperidyl]-2-(1,5,5-trimethyl-3-piperidyl)indazole (5.18 g, 15.22 mmol) was dissolved in DCM (75 mL) and TEA (2.31 g, 22.83 mmol, 3.18 mL) was added thereto. Di-tert-butyl dicarbonate (4.15 g, 19.02 mmol, 4.37 mL) was added dropwise to the previous solution and the resulting mixture was stirred overnight. The reaction mixture was concentrated in vacuum and the residue was purified by column chromatography (gradient MeOH in MTBE from 0% to 100%) to obtain tert-butyl (2R,5S)-5-methyl-2-[2-(1,5,5-trimethyl-3-piperidyl)indazol-6-yl]piperidine-1-carboxylate (740 mg, 1.68 mmol, 11.04% yield) and tert-butyl (2R,5S)-5-methyl-2-[2-(1,5,5-trimethyl-3-piperidyl)indazol-6-yl]piperidine-1-carboxylate (2.03 g, 4.61 mmol, 30.27% yield).

LCMS(ESI): [M]⁺ m/z: calcd 440.2; found 441.2; Rt=1.431 min.

### Step 6: Chiral Separation

Racemic tert-butyl (2R,5S)-5-methyl-2-[2-(1,5,5-trimethyl-3-piperidyl)indazol-6-yl]piperidine-1-carboxylate (2.03 g, 4.61 mmol) was chiral separated (Column: Chiralpak IA (250×20 mm, 5 mkm); Mobile phase: Hexane-IPA-MeOH, 80-10-10; Flow Rate: 14 mL/min) to obtain tert-butyl (2R,5S)-5-methyl-2-[2-[(3S)-1,5,5-trimethyl-3-piperidyl]indazol-6-yl]piperidine-1-carboxylate (732.4 mg, 1.66 mmol, 36.08% yield) (RT=23.90 min) and tert-butyl (2R,5S)-5-methyl-2-[2-[(3R)-1,5,5-trimethyl-3-piperidyl]indazol-6-yl]piperidine-1-carboxylate (792.51 mg, 1.80 mmol, 39.04% yield) (RT=16.88 min).

Rel Time for A in analytical conditions (column: IA, Hexane-IPA-MeOH, 80-10-10, 0.6 ml/min as mobile phase) 27.03 min and for B 19.15 min.

### A: Retention Time: 27.03 Min

LCMS(ESI): [M]⁺ m/z: calcd 440.2; found 441.2; Rt=1.266 min.

### B: Retention Time: 19.15 Min

LCMS(ESI): [M]⁺ m/z: calcd 440.2; found 441.2; Rt=1.255 min.

### Step 7: Synthesis of 6-((2R,5S)-5-methylpiperidin-2-yl)-2-(1,5,5-trimethylpiperidin-3-yl)-2H-indazole

A: tert-Butyl (2R,5S)-5-methyl-2-[2-[(35)-1,5,5-trimethyl-3-piperidyl]indazol-6-yl]piperidine-1-carboxylate (732.4 mg, 1.66 mmol) was dissolved in MeOH (5 mL) and HCl/dioxane (5 mL) was added thereto. The resulting mixture was stirred for 4 hr. The reaction mixture was concentrated in vacuum to obtain 6-[(2R,5S)-5-methyl-2-piperidyl]-2-[(35)-1,5,5-trimethyl-3-piperidyl]indazole (603.3 mg, 1.46 mmol, 87.79% yield, 2HCl).

LCMS(ESI): [M]⁺ m/z: calcd 340.2; found 341.2; Rt=0.556 min.

B: tert-Butyl (2R,5S)-5-methyl-2-[2-[(3R)-1,5,5-trimethyl-3-piperidyl]indazol-6-yl]piperidine-1-carboxylate (792.51 mg, 1.80 mmol) was dissolved in MeOH (5 mL) and HCl/dioxane (5 mL) was added thereto. The resulting mixture was stirred for 4 hr. The reaction mixture was concentrated in vacuum to obtain 6-[(2R,5S)-5-methyl-2-piperidyl]-2-[(3R)-1,5,5-trimethyl-3-piperidyl]indazole (659.7 mg, 1.60 mmol, 88.72% yield, 2HCl).

LCMS(ESI): [M]⁺ m/z: calcd 340.2; found 341.2; Rt=0.561 min.

### Step 8: Synthesis of N-(6-amino-5-ethylpyridin-3-yl)-2-((2R,5S)-5-methyl-2-(2-(1,5,5-trimethylpiperidin-3-yl)-2H-indazol-6-yl)piperidin-1-yl)-2-oxoacetamide (, Compound 114 and Compound 78)

Prepared by general procedure scheme 6.2 step 5. Yield: 243.2 mg (63.03%).

HPLC conditions: Column: SunFire C18 100*19 mm, 5 microM; 2-10 min 60-80% MeOH+NH₃30 ml/min; (loading pump 4 ml/min MeOH).

Compound 114: ¹H NMR (600 MHz, DMSO-d₆) δ (ppm) 0.98 (m, 6H), 1.12 (m, 6H), 1.35 (m, 1H), 1.79 (m, 5H), 2.05 (m, 1H), 2.20 (s, 5H), 2.40 (m, 4H), 2.93 (m, 1H), 3.97 (m, 1H), 4.76 (m, 1H), 5.61 (m, 3H), 7.00 (m, 1H), 7.48 (m, 2H), 7.66 (m, 1H), 8.03 (m, 1H), 8.40 (s, 1H), 10.52 (m, 1H).

LCMS(ESI): [M]⁺ m/z: calcd 531.2; found 532.2; Rt=1.945 min.

Prepared by general procedure scheme 6.2 step 5. Yield: 225.3 mg (58.40%).

HPLC conditions: Column: SunFire C18 100*19 mm, 5 microM; 2-10 min 60-80% MeOH+NH₃30 ml/min; (loading pump 4 ml/min MeOH).

Compound 78: ¹H NMR (600 MHz, DMSO-d₆) δ (ppm) 0.98 (m, 6H), 1.12 (m, 6H), 1.35 (m, 1H), 1.79 (m, 5H), 2.05 (m, 1H), 2.20 (s, 5H), 2.39 (m, 4H), 2.93 (m, 1H), 4.03 (m, 1H), 4.75 (m, 1H), 5.61 (m, 3H), 7.00 (m, 1H), 7.49 (m, 2H), 7.66 (m, 1H), 8.03 (m, 1H), 8.40 (s, 1H), 10.52 (m, 1H).

LCMS(ESI): [M]⁺ m/z: calcd 531.2; found 532.2; Rt=1.918 min.

### Example 71. Compound 102 2-methoxy-5-(2-((2R,5S)-5-methyl-2-(2-((R)-1-methylpyrrolidin-3-yl)-2H-indazol-6-yl)piperidin-1-yl)-2-oxoacetamido)nicotinamide

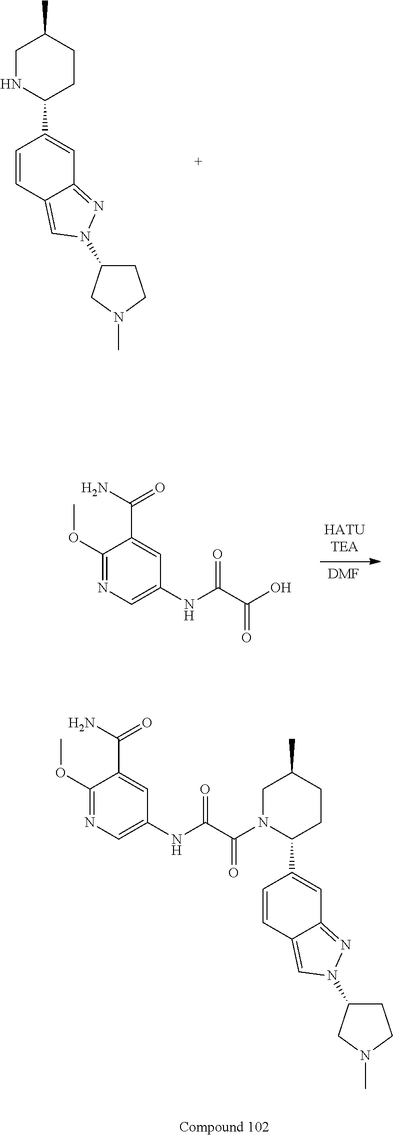

The synthesis of 6-((2R,5S)-5-methylpiperidin-2-yl)-2-((R)-1-methylpyrrolidin-3-yl)-2H-indazole is given by general procedure scheme 6.2 step 3. Yield: 510 mg of crude.

LCMS(ESI): [M]⁺ m/z: calcd 298.2; found 299.2; Rt=0.622 min.

Prepared by general procedure scheme 6.2 step 5. Yield: 62.8 mg (24.05%).

HPLC conditions: Column: SunFire C18 100*19 mm, 5 microM; 2-10 min 30-60% MeOH+NH₃; (loading pump 4 ml/min MeOH).

Compound 102: ¹H NMR (600 MHz, DMSO-d₆) δ (ppm) 0.72-1.07 (m, 3H), 1.27-1.42 (m, 1H), 1.68-1.81 (m, 1H), 1.84-1.96 (m, 1H), 2.03-2.36 (m, 6H), 2.39-2.46 (m, 1H), 2.51-2.58 (m, 2H), 2.80-2.87 (m, 2H), 2.90-2.97 (m, 1H), 3.46-4.07 (m, 4H), 5.11-5.75 (m, 2H), 6.94-7.11 (m, 1H), 7.50-7.58 (m, 1H), 7.62-7.82 (m, 3H), 8.17-8.61 (m, 3H), 10.99-11.16 (m, 1H).

LCMS(ESI): [M]⁺ m/z: calcd 519.2; found 520.2; Rt=2.092 min.

### Example 72. Compound 103 2-methoxy-5-(2-((2R,5S)-5-methyl-2-(2-((S)-1-methylpyrrolidin-3-yl)-2H-indazol-6-yl)piperidin-1-yl)-2-oxoacetamido)nicotinamide

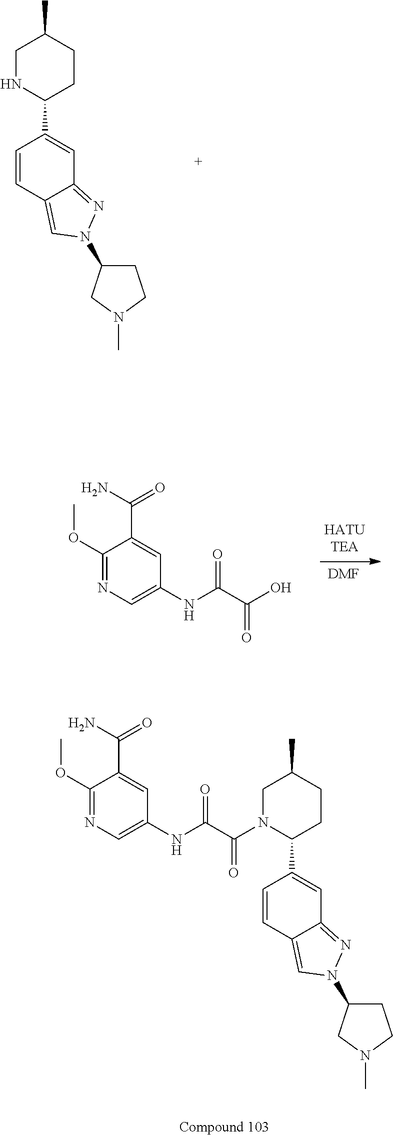

The synthesis of 6-((2R,5S)-5-methylpiperidin-2-yl)-2-((S)-1-methylpyrrolidin-3-yl)-2H-indazole is given prepared by general procedure scheme 6.2 step 3. Yield: 234 mg of crude.

LCMS(ESI): [M]⁺ m/z: calcd 298.2; found 299.2; Rt=0.433 min.

Prepared by general procedure scheme 6.2 step 5. Yield: 24.6 mg (21.08%).

HPLC conditions: Column: SunFire C18 100*19 mm, 5 microM; 2-10 min 30-50% MeCN+NH₃; (loading pump 4 ml/min MeCN).

Compound 103: ¹H NMR (500 MHz, DMSO-d₆) δ (ppm) 0.75-1.08 (m, 3H), 1.22-1.48 (m, 1H), 1.66-1.82 (m, 1H), 1.82-1.99 (m, 1H), 2.02-2.19 (m, 1H), 2.20-2.36 (m, 3H), 2.37-2.43 (m, 3H), 2.62-2.71 (m, 1H), 2.78-3.08 (m, 4H), 3.47-3.51 (m, 0.7H), 3.91-3.98 (m, 3H), 4.02-4.07 (m, 0.3H), 5.07-5.73 (m, 2H), 7.02 (dd, 1H), 7.49-7.56 (m, 1H), 7.66-7.80 (m, 3H), 8.36-8.49 (m, 2H), 8.50-8.64 (m, 1H), 10.85-11.30 (m, 1H).

LCMS(ESI): [M]⁺ m/z: calcd 519.2; found 520.2; Rt=2.190 min.

### Example 73. Compound 93 N-(6-amino-5-ethyl-3-pyridyl)-2-oxo-2-[rac-(2R,5S)-5-methyl-2-(2-tetrahydrofuran-3-ylindazol-6-yl)-1-piperidyl]acetamide

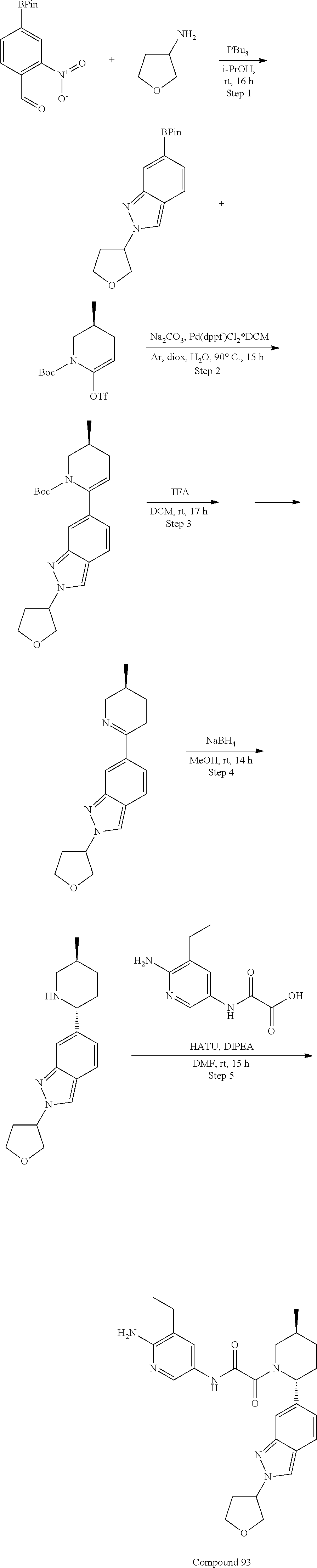

### Step 1: The Synthesis of 2-tetrahydrofuran-3-yl-6-(4,4,5,5-tetramethyl-1,3,2-dioxaborolan-2-yl)indazole

Prepared by general procedure Scheme 6.2 Step 1 The crude product was purified by FCC (MTBE in HEX from 0% to 100%).

Yield: 1.1 g (38.8%)

LCMS(ESI): [M+H]⁺ m/z: calcd 314.2; found 315.0; Rt=1.376 min.

### Step 2: The Synthesis of tert-butyl rac-(3S)-3-methyl-6-(2-tetrahydrofuran-3-ylindazol-6-yl)-3,4-dihydro-2H-pyridine-1-carboxylate

Prepared by general procedure Scheme 6.2 Step 2 Yield: 2.1 g (crude)

LCMS(ESI): [M+H]⁺ m/z: calcd 383.2; found 384.2; Rt=1.443 min.

### Step 3: The Synthesis of 6-[rac-(3S)-3-methyl-2,3,4,5-tetrahydropyridin-6-yl]-2-tetrahydrofuran-3-yl-indazole

Prepared by general procedure Scheme 6.2 Step 3 Yield: 0.7 g (45.11%)

LCMS(ESI): [M+H]⁺ m/z: calcd 283.2; found 284.2; Rt=0.645 min.

### Step 4: The Synthesis of 6-[rac-(2R,5S)-5-methyl-2-piperidyl]-2-tetrahydrofuran-3-yl-indazole

Prepared by general procedure Scheme 6.2 Step 4 (Method B) Yield: 0.4 g (56.7%)

LCMS(ESI): [M+H]⁺ m/z: calcd 285.2; found 286.2; Rt=0.655 min.

### Step 5: Synthesis of N-(6-amino-5-ethyl-3-pyridyl)-2-oxo-2-[rac-(2R,5S)-5-methyl-2-(2-tetrahydrofuran-3-ylindazol-6-yl)-1-piperidyl]acetamide (Compound 93)

Prepared by general procedure Scheme 6.2 Step 5 HPLC conditions: 2-10 min 10-50% methanol+NH₃, 30 ml/min (loading pump 4 ml methanol+NH₃) column: YMC-ACTUS TRIART C18 100*20 5 microM.

Yield: 56 mg (33.5%)

¹H NMR (600 MHz, dmso) δ 1.01-1.05 (m, 3H), 1.06-1.14 (m, 3H), 1.28-1.42 (m, 1H), 1.69-1.81 (m, 1H), 1.81-1.93 (m, 1H), 2.01-2.22 (m, 1H), 2.23-2.37 (m, 2H), 2.39-2.43 (m, 2H), 2.76-3.24 (m, 1H), 3.37-3.50 (m, 1H), 3.85-3.90 (m, 1H), 3.93-4.06 (m, 2H), 4.06-4.13 (m, 2H), 5.18-5.60 (m, 2H), 5.61-5.66 (m, 2H), 6.94-7.11 (m, 1H), 7.43-7.57 (m, 2H), 7.66-7.72 (m, 1H), 7.98-8.09 (m, 1H), 8.34-8.41 (m, 1H), 10.43-10.61 (m, 1H).

LCMS(ESI): [M+H]⁺ m/z: calcd 476.3; found 477.2; Rt=2.238 min.

### Example 74. Compound 17 N-(6-amino-5-ethyl-3-pyridyl)-2-oxo-2-[(2R,5S)-5-methyl-2-(2-tetrahydropyran-4-ylindazol-6-yl)-1-piperidyl]acetamide

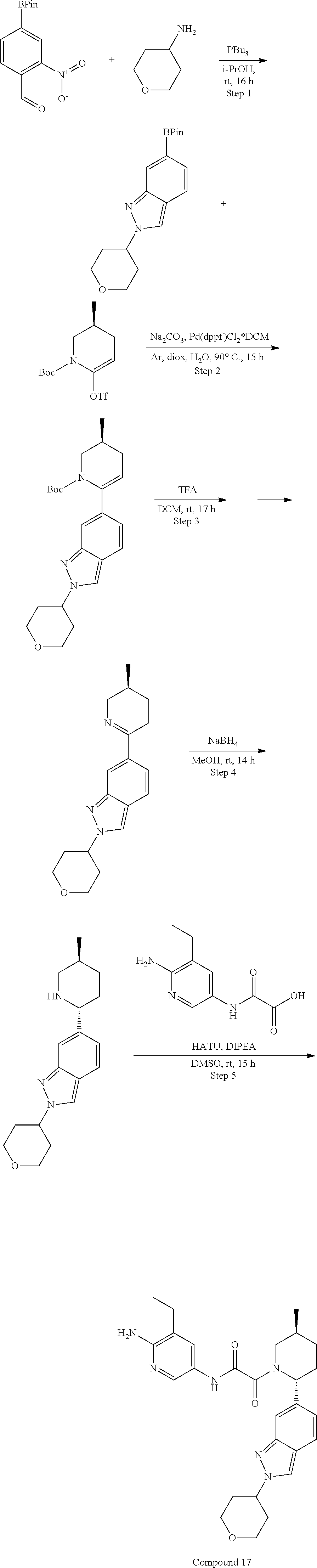

### Step 1: The Synthesis of 2-tetrahydropyran-4-yl-6-(4,4,5,5-tetramethyl-1,3,2-dioxaborolan-2-yl)indazole

Prepared by general procedure Scheme 6.2 Step 1

FCC conditions: FCC (gradient EtOAc in hexane from 0% to 100%)

Yield: 1.64 g (69.39%)

LCMS(ESI): [M+2H]⁺ m/z: calcd 328.2; found 329.0; Rt=1.168 min.

### Step 2: The Synthesis of tert-butyl (3S)-3-methyl-6-(2-tetrahydropyran-4-ylindazol-6-yl)-3,4-dihydro-2H-pyridine-1-carboxylate

Prepared by general procedure Scheme 6.2 Step 2

Yield: 2.07 g (crude)

LCMS(ESI): [M+H]⁺ m/z: calcd 397.2; found 398.2; Rt=1.352 min.

### Step 3: The Synthesis of 6-[(3S)-3-methyl-2,3,4,5-tetrahydropyridin-6-yl]-2-tetrahydropyran-4-yl-indazole

Prepared by general procedure Scheme 6.2 Step 3

Yield: 842 mg (54.3%)

LCMS(ESI): [M+H]⁺ m/z: calcd 297.2; found 298.2; Rt=0.732 min.

### Step 4: The Synthesis of 6-[(2R,5S)-5-methyl-2-piperidyl]-2-tetrahydropyran-4-yl-indazole

Prepared by general procedure Scheme 6.2 Step 4 (Method B)

Yield: 832 mg (98.15%)

LCMS(ESI): [M+H]⁺ m/z: calcd 299.2; found 300.2; Rt=0.854 min.

### Step 5: Synthesis of N-(6-amino-5-ethyl-3-pyridyl)-2-oxo-2-[(2R,5S)-5-methyl-2-(2-tetrahydropyran-4-ylindazol-6-yl)-1-piperidyl]acetamide (Compound 17)

Prepared by general procedure Scheme 6.2 Step 5

HPLC conditions: 2-10 min 10-50% MeOH+NH₃, 30 ml/min ((loading pump 4 ml MeOH+NH₃), column: YMC-ACTUS TRIART C18 100*20 5 microM2

Yield: 72 mg (30%)

¹H NMR (600 MHz, dmso) δ 0.97-1.05 (m, 3H), 1.05-1.15 (m, 3H), 1.29-1.41 (m, 1H), 1.64-1.82 (m, 1H), 1.83-1.94 (m, 1H), 2.00-2.15 (m, 5H), 2.24-2.33 (m, 1H), 2.36-2.46 (m, 2H), 2.73-3.28 (m, 1H), 3.46-3.98 (m, 3H), 3.98-4.07 (m, 2H), 4.68-4.76 (m, 1H), 5.18-5.68 (m, 3H), 6.94-7.09 (m, 1H), 7.42-7.57 (m, 2H), 7.65-7.72 (m, 1H), 7.96-8.11 (m, 1H), 8.35-8.43 (m, 1H), 10.43-10.60 (m, 1H).

LCMS(ESI): [M+H]⁺ m/z: calcd 490.3; found 491.2; Rt=2.290 min.

### Example 75. Compound 7 2-methoxy-5-[[2-oxo-2-[(2R,5S)-2-[2-[2-(dimethylamino)ethyl]indazol-6-yl]-5-methyl-1-piperidyl]acetyl]amino]pyridine-3-carboxamide

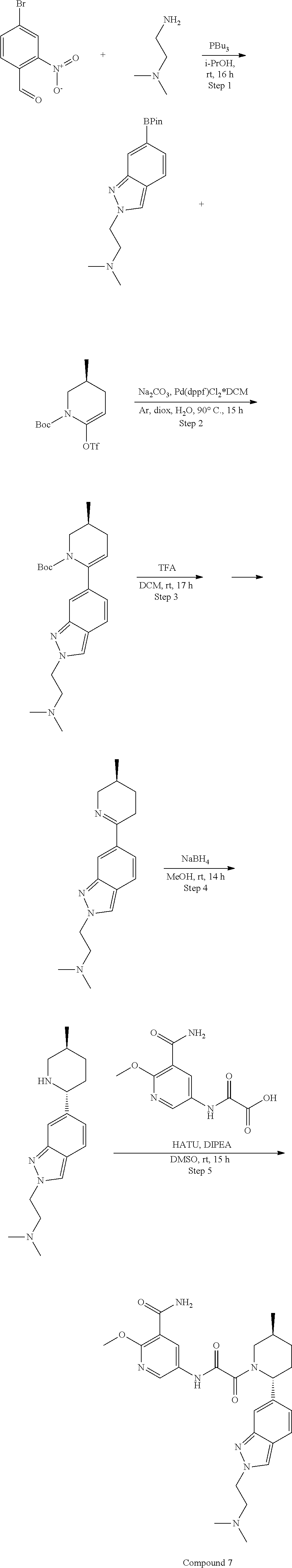

### Step 1: The Synthesis of N,N-dimethyl-2-[6-(4,4,5,5-tetramethyl-1,3,2-dioxaborolan-2-yl)indazol-2-yl]ethanamine

Prepared by general procedure Scheme 6.2 Step 1

The crude product was purified by FCC (MeOH in MTBE from 2% to 67%).

Yield: 1.45 g (50.98%)

LCMS(ESI): [M+H]⁺ m/z: calcd 315.2; found 316.2; Rt=1.056 min.

### Step 2: The Synthesis of tert-butyl (3S)-6-[2-[2-(dimethylamino)ethyl]indazol-6-yl]-3-methyl-3,4-dihydro-2H-pyridine-1-carboxylate

Prepared by general procedure Scheme 6.2 Step 2

Yield: 2.6 g (crude)

LCMS(ESI): [M+H]⁺ m/z: calcd 384.2; found 385.2; Rt=1.011 min.

### Step 3: The Synthesis of N,N-dimethyl-2-[6-[(3S)-3-methyl-2,3,4,5-tetrahydropyridin-6-yl]indazol-2-yl]ethanamine

Prepared by general procedure Scheme 6.2 Step 3

Yield: 0.9 g (46.8%)

LCMS(ESI): [M+H]⁺ m/z: calcd 284.2; found 285.2; Rt=0.407 min.

### Step 4: The Synthesis of N,N-dimethyl-2-[6-[(2R,5S)-5-methyl-2-piperidyl]indazol-2-yl]ethanamine

Prepared by general procedure Scheme 6.2 Step 4 (Method B)

Yield: 0.7 g (77.2%)

LCMS(ESI): [M+H]⁺ m/z: calcd 286.2; found 287.2; Rt=0.414 min.

### Step 5: Synthesis of 2-methoxy-5-[[2-oxo-2-[(2R,5S)-2-[2-[2-(dimethylamino)ethyl]indazol-6-yl]-5-methyl-1-piperidyl]acetyl]amino]pyridine-3-carboxamide (Compound 7)

Prepared by general procedure Scheme 6.2 Step 5

HPLC conditions: 2-10 min 10-50% methanol+NH₃, 30 ml/min (loading pump 4 ml methanol+NH₃) column: YMC-ACTUS TRIART C18 100*20 5 microM

Yield: 42 mg (23.7%)

¹H NMR (600 MHz, dmso) δ 0.70-1.08 (m, 3H), 1.27-1.45 (m, 1H), 1.65-1.82 (m, 1H), 1.82-1.95 (m, 1H), 2.00-2.15 (m, 1H), 2.15-2.24 (m, 6H), 2.24-2.35 (m, 1H), 2.73-2.79 (m, 2H), 2.80-3.26 (m, 1H), 3.40-3.53 (m, 0.7H), 3.90-3.98 (m, 3H), 4.01-4.06 (m, 0.3H), 4.42-4.53 (m, 2H), 5.18-5.79 (m, 1H), 6.93-7.09 (m, 1H), 7.48-7.55 (m, 1H), 7.61-7.74 (m, 2H), 7.73-7.78 (m, 1H), 8.27-8.37 (m, 1H), 8.39-8.50 (m, 1H), 8.50-8.63 (m, 1H), 10.90-11.20 (m, 1H).

LCMS(ESI): [M+H]⁺ m/z: calcd 507.3; found 508.2; Rt=2.002 min.

### Example 76. Compound 107 2-methoxy-5-[[2-oxo-2-[(2R,5S)-5-methyl-2-(2-tetrahydropyran-4-ylindazol-6-yl)-1-piperidyl]acetyl]amino]pyridine-3-carboxamide

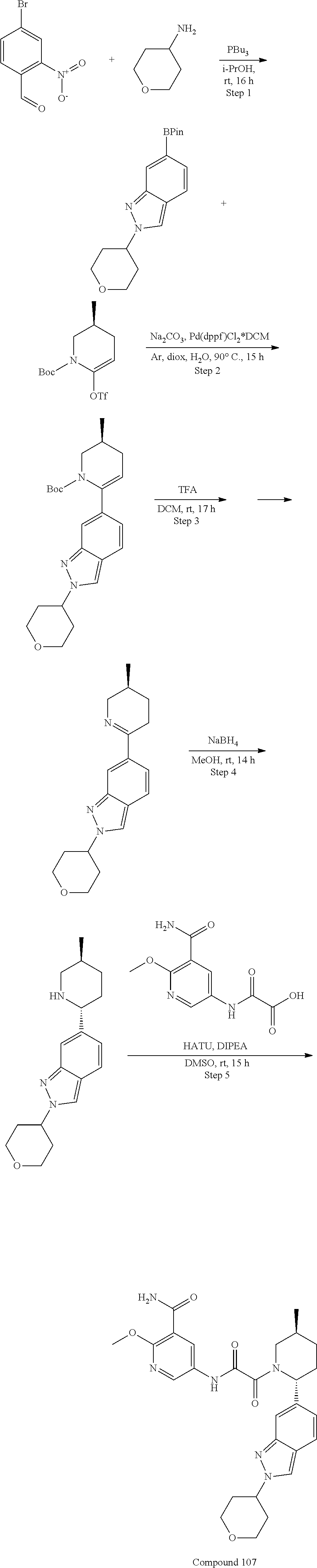

### Step 1: The Synthesis of 2-tetrahydropyran-4-yl-6-(4,4,5,5-tetramethyl-1,3,2-dioxaborolan-2-yl)indazole

Prepared by general procedure Scheme 6.2 Step 1

FCC conditions: FCC (gradient EtOAc in hexane from 0% to 100%)

Yield: 1.64 g (69.39%)

LCMS(ESI): [M+2H]⁺ m/z: calcd 328.2; found 329.0; Rt=1.168 min.

### Step 2: The Synthesis of tert-butyl (3S)-3-methyl-6-(2-tetrahydropyran-4-ylindazol-6-yl)-3,4-dihydro-2H-pyridine-1-carboxylate

Prepared by general procedure Scheme 6.2 Step 2

Yield: 2.07 g (crude)

LCMS(ESI): [M+H]⁺ m/z: calcd 397.2; found 398.2; Rt=1.352 min.

### Step 3: The Synthesis of 6-[(3S)-3-methyl-2,3,4,5-tetrahydropyridin-6-yl]-2-tetrahydropyran-4-yl-indazole

Prepared by general procedure Scheme 6.2 Step 3

Yield: 842 mg (54.3%)

LCMS(ESI): [M+H]⁺ m/z: calcd 297.2; found 298.2; Rt=0.732 min.

### Step 4: The Synthesis of 6-[(2R,5S)-5-methyl-2-piperidyl]-2-tetrahydropyran-4-yl-indazole

Prepared by general procedure Scheme 6.2 Step 4 (Method B)

Yield: 832 mg (98.15%)

LCMS(ESI): [M+H]⁺ m/z: calcd 299.2; found 300.2; Rt=0.854 min.

### Step 5: Synthesis of 2-methoxy-5-[[2-oxo-2-[(2R,5S)-5-methyl-2-(2-tetrahydropyran-4-ylindazol-6-yl)-1-piperidyl]acetyl]amino]pyridine-3-carboxamide (Compound 107)

Prepared by general procedure Scheme 6.2 Step 5

HPLC conditions: (2-10 min 30-60 MeOH+NH₃30 ml/min)

Yield: 81.8 mg (31.36%)

¹H NMR (600 MHz, dmso) δ 0.73-1.08 (m, 3H), 1.30-1.44 (m, 1H), 1.70-1.82 (m, 1H), 1.82-1.95 (m, 1H), 2.01-2.17 (m, 5H), 2.20-2.34 (m, 1H), 2.83-3.29 (m, 1H), 3.46-3.55 (m, 2.6H), 3.90-3.97 (m, 3H), 3.97-4.05 (m, 2.4H), 4.69-4.77 (m, 1H), 5.20-5.69 (m, 1H), 6.94-7.09 (m, 1H), 7.50-7.57 (m, 1H), 7.64-7.77 (m, 3H), 8.35-8.62 (m, 3H), 10.94-11.20 (m, 1H).

LCMS(ESI): [M+H]⁺ m/z: calcd 520.3; found 521.2; Rt=2.874 min.

### Example 77. Compound 97 2-methoxy-5-[[2-oxo-2-[rac-(2R,5S)-5-methyl-2-(2-tetrahydrofuran-3-ylindazol-6-yl)-1-piperidyl]acetyl]amino]pyridine-3-carboxamide

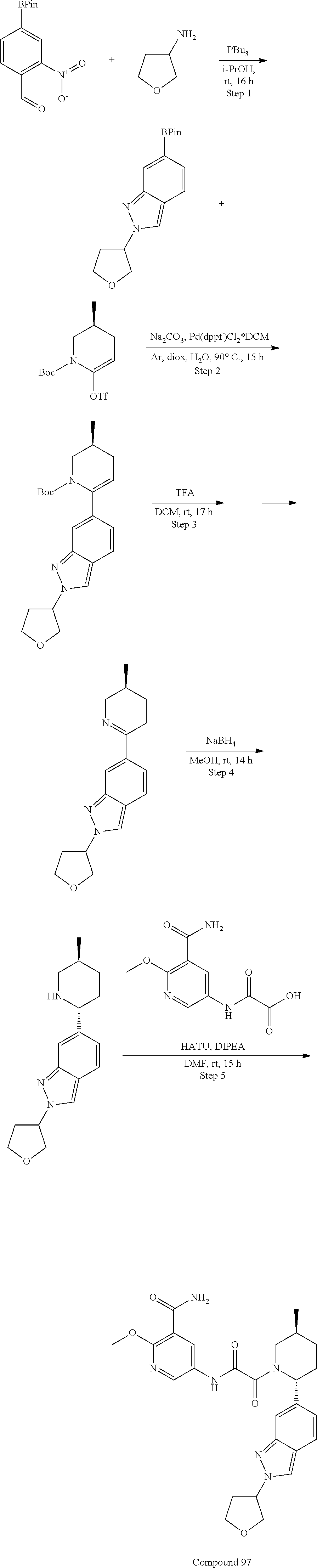

### Step 1: The Synthesis of 2-tetrahydrofuran-3-yl-6-(4,4,5,5-tetramethyl-1,3,2-dioxaborolan-2-yl)indazole

Prepared by general procedure Scheme 6.2 Step 1

The crude product was purified by FCC (MTBE in HEX from 0% to 100%).

Yield: 1.1 g (38.8%)

LCMS(ESI): [M+H]⁺ m/z: calcd 314.2; found 315.0; Rt=1.376 min.

### Step 2: The Synthesis of tert-butyl rac-(3S)-3-methyl-6-(2-tetrahydrofuran-3-ylindazol-6-yl)-3,4-dihydro-2H-pyridine-1-carboxylate

Prepared by general procedure Scheme 6.2 Step 2

Yield: 2.1 g (crude)

LCMS(ESI): [M+H]⁺ m/z: calcd 383.2; found 384.2; Rt=1.443 min.

### Step 3: The Synthesis of 6-[rac-(3S)-3-methyl-2,3,4,5-tetrahydropyridin-6-yl]-2-tetrahydrofuran-3-yl-indazole

Prepared by general procedure Scheme 6.2 Step 3

Yield: 0.7 g (45.11%)

LCMS(ESI): [M+H]⁺ m/z: calcd 283.2; found 284.2; Rt=0.645 min.

### Step 4: The Synthesis of 6-[rac-(2R,5S)-5-methyl-2-piperidyl]-2-tetrahydrofuran-3-yl-indazole

Prepared by general procedure Scheme 6.2 Step 4 (Method B)

Yield: 0.4 g (56.7%)

LCMS(ESI): [M+H]⁺ m/z: calcd 285.2; found 286.2; Rt=0.655 min.

### Step 5: Synthesis of 2-methoxy-5-[[2-oxo-2-[rac-(2R,5S)-5-methyl-2-(2-tetrahydrofuran-3-ylindazol-6-yl)-1-piperidyl]acetyl]amino]pyridine-3-carboxamide (Compound 97)

Prepared by general procedure Scheme 6.2 Step 5

HPLC conditions: 2-10 min 30-60% methanol+NH₃, 30 ml/min (loading pump 4 ml methanol+NH₃) column: YMC-ACTUS TRIART C18 100*20 5 microM

Yield: 79 mg (44.5%).

¹H NMR (600 MHz, dmso) δ 0.72-1.09 (m, 3H), 1.29-1.43 (m, 1H), 1.66-1.82 (m, 1H), 1.83-1.96 (m, 1H), 2.04-2.22 (m, 1H), 2.24-2.36 (m, 1H), 2.38-2.47 (m, 2H), 2.81-3.29 (m, 1H), 3.44-3.54 (m, 0.6H), 3.85-3.90 (m, 1H), 3.91-3.97 (m, 3H), 3.98-4.04 (m, 1.4H), 4.05-4.10 (m, 2H), 5.09-5.83 (m, 2H), 6.96-7.11 (m, 1H), 7.49-7.58 (m, 1H), 7.65-7.81 (m, 3H), 8.35-8.49 (m, 2H), 8.49-8.62 (m, 1H), 10.99-11.14 (m, 1H).

LCMS(ESI): [M+H]⁺ m/z: calcd 506.2; found 507.0; Rt=2.808 min.

### Scheme 7.1

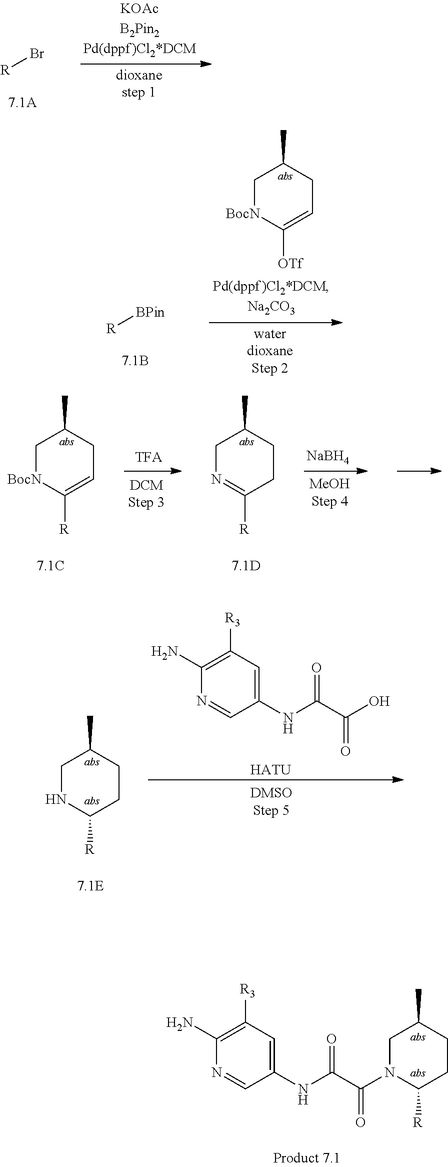

### Step 1: The Synthesis of 7.1B

7.1A (1 eq), B₂Pin₂ (1.1 eq) and KOAc (2 eq) were mixed in dioxane. The resulting mixture was evacuated and then backfilled with argon, this operation was repeated three times, then Pd(dppf)Cl₂*DCM (0.05 eq) was added under argon. The reaction mixture was stirred under argon at 90° C. for 14 hr, then cooled and filtered. The filter cake was washed with dioxane twice. The solvent was evaporated to afford 7.1B.

### Step 2: The Synthesis of 7.1C

7.1B (1 eq), tert-butyl (3S)-3-methyl-6-(trifluoromethylsulfonyloxy)-3,4-dihydro-2H-pyridine-1-carboxylate (1.2 eq), sodium carbonate (3 eq) were mixed together in dioxane-water mixture (3:1). The resulting mixture was evacuated and then backfilled with argon. This operation was repeated two times, then Pd(dppf)Cl₂*DCM (819.86 mg, 1.00 mmol) was added and the reaction mixture was stirred under argon at 90° C. overnight, then cooled down and concentrated in vacuum. The residue was diluted with MTBE and stirred for 0.5 hr. After the most of the residue had dissolved, anhydrous sodium sulphate was added, and the resulting mixture was filtered. The filter cake was additionally washed with MTBE (5*50 ml) and discarded. The filtrate was concentrated in vacuum to afford 7.1C.

### Step 3: The Synthesis of 7.1D

A solution of 7.1C (1.0 equiv) in TFA (50.0 mL) was stirred at 25° C. for 1 hr, and then concentrated in vacuum. Crushed ice (10 g) was added to the residue, and the resulting mixture was basified to pH 10 with 10% aqueous potassium carbonate solution and extracted with DCM (2*100.0 mL). The combined organic extracts were dried over sodium sulphate and concentrated under reduced pressure to afford 5.2D.

### Step 4: The Synthesis of 7.1E

7.1D (1 eq) was dissolved in MeOH and the resulting solution was cooled to 0° C. in an ice bath. Sodium borohydride (2 eq) was added portion wise to the previous solution. After addition completed, the reaction mixture was allowed to warm to rt and stirred overnight. Water was added to the reaction mixture and the resulting mixture was concentrated in vacuum. The residue was diluted with water and the resulting mixture was extracted with DCM twice, dried over Na₂SO₄, filtered and evaporated to obtain 7.1E.

### Step 5A: Synthesis of Product 7.1

7.1E (1 eq), oxamic acid (1 eq) and TEA (2.5 eq+1.0 eq per each acid eq, if amine salt used) were mixed together in DMF. HATU (1.5 eq) was added thereto and the resulting mixture was stirred overnight. The reaction mixture was concentrated in vacuum and the residue was purified by HPLC to obtain Product 7.1.

### Step 5B: Synthesis of Product 7.1

DIPEA (2.5 eq+1.0 eq per each acid eq, if amine salt used) was added to the solution of respective amine or it salt (7.1E) (1 eq) and oxamic acid (1 eq) in DMF. The resulting mixture was stirred for 5 min followed by the addition of the solution of HATU (1.1 eq) in DMF. Then, the reaction mixture was stirred overnight at rt. After the completion of the reaction, monitored by LCMS, the resulting suspension was concentrated under reduced pressure. The obtained filtrate was subjected to HPLC (Waters SunFire C18 19*100 5 mkm column and H₂O-MeOH as a mobile phase) to afford pure product (Product 7.1).

### Example 78. Compound 58 N-(6-amino-5-ethylpyridin-3-yl)-2-((2R,5S)-5-methyl-2-(3-((1-methylpiperidin-4-yl)oxy)phenyl)piperidin-1-yl)-2-oxoacetamide

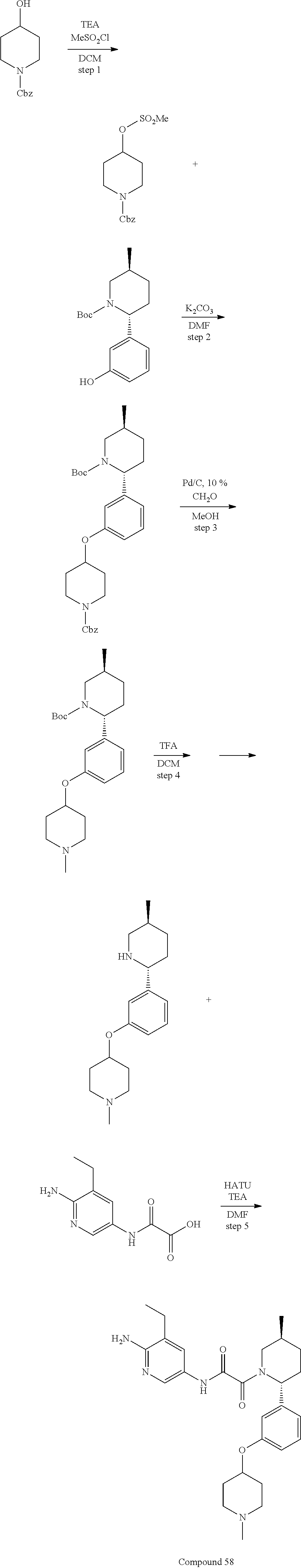

### Step 1: Synthesis of benzyl 4-((methylsulfonyl)oxy)piperidine-1-carboxylate

The solution of benzyl 4-hydroxypiperidine-1-carboxylate (1.75 g, 7.44 mmol) and TEA (1.13 g, 11.16 mmol, 1.56 mL) in DCM (14.69 mL) was cooled to 0° C. and at this temperature methanesulfonyl chloride (937.23 mg, 8.18 mmol, 634.55 μL) was added dropwise. The reaction mixture was heated to rt and stirred for 12 hr. The reaction mixture was extracted with aq. NaHCO₃, aq. NaHSO₄, dried over Na₂SO₄, filtered and concentrated on vacuum to obtain benzyl 4-((methylsulfonyl)oxy)piperidine-1-carboxylate (2.1 g, 6.70 mmol, 90.1% yield)

¹H NMR (400 MHz, CDCl₃) δ (ppm) 1.84 (m, 2H), 1.94 (m, 2H), 3.01 (s, 3H), 3.40 (m, 2H), 3.74 (m, 2H), 4.88 (m, 1H), 5.11 (s, 2H), 7.33 (m, 5H).

### Step 2: Synthesis of (2R,5S)-tert-butyl 2-(3-((1-((benzyloxy)carbonyl)piperidin-4-yl)oxy)phenyl)-5-methylpiperidine-1-carboxylate

tert-Butyl (2R,5S)-2-(3-hydroxyphenyl)-5-methyl-piperidine-1-carboxylate (1.4 g, 4.80 mmol), benzyl 4-methylsulfonyloxypiperidine-1-carboxylate (1.96 g, 6.25 mmol) and potassium carbonate, anhydrous, 99% (1.66 g, 12.01 mmol, 724.95 μL) was mixed together in DMF (20 mL) and heated at 100° C. for 16 hr. The reaction mixture was concentrated on vacuum. The obtained residue was dissolved in EtOAc/H₂O, the EtOAc layer was separated and the aqueous layer was extracted twice with EtOAc. The combined organic layers was dried over Na₂SO₄, filtered and concentrated on vacuum. The residue was purified by FCC to obtain tert-butyl (2R,5S)-2-[3-[(1-benzyloxycarbonyl-4-piperidyl)oxy]phenyl]-5-methyl-piperidine-1-carboxylate (280 mg, 550.48 mol, 11.46% yield).

LCMS(ESI): [M-Boc]⁺ m/z: calcd 408.2; found 409.2; Rt=1.853 min.

### Step 3: Synthesis of (2R,5S)-tert-butyl 5-methyl-2-(3-((1-methylpiperidin-4-yl)oxy)phenyl)piperidine-1-carboxylate

The mixture of tert-butyl (2R,5S)-2-[3-[(1-benzyloxycarbonyl-4-piperidyl)oxy]phenyl]-5-methyl-piperidine-1-carboxylate (280 mg, 550.48 mol), palladium, 10% on carbon, Type 487, dry (58.58 mg, 550.48 mol) and formaldehyde, 37% w/w aq. soln., stab. with 7-8% MeOH (82.64 mg, 2.75 mmol, 76.31 μL) in MeOH (3 mL) was stirred at 25° C. for 96 hr under H₂ atmosphere. The mixture was filtrated and filtrate was evaporated to give tert-butyl (2R,5S)-5-methyl-2-[3-[(1-methyl-4-piperidyl)oxy]phenyl]piperidine-1-carboxylate (200 mg, crude).

LCMS(ESI): [M]⁺ m/z: calcd 388.2; found 389.2; Rt=0.983 min.

### Step 4: Synthesis of 1-methyl-4-(3-((2R,5S)-5-methylpiperidin-2-yl)phenoxy)piperidine

tert-Butyl (2R,5S)-5-methyl-2-[3-[(1-methyl-4-piperidyl)oxy]phenyl]piperidine-1-carboxylate (200 mg, 514.74 mol) was dissolved in TFA (1 mL) and DCM (1 mL) and was stirred at 25° C. for 1 hr. Then basified to pH 11-12 with 10% aqueous sodium hydroxide solution and extracted with DCM. The combined organic extracts were dried over sodium sulfate and concentrated in vacuum to afford 1-methyl-4-[3-[(2R,5S)-5-methyl-2-piperidyl]phenoxy]piperidine (100 mg, 346.71 mol, 67.36% yield) as light-yellow gum, which was directly used in the next step.

LCMS(ESI): [M]⁺ m/z: calcd 288.2; found 289.2; Rt=0.535 min.

### Step 5: Synthesis of N-(6-amino-5-ethylpyridin-3-yl)-2-((2R,5S)-5-methyl-2-(3-((1-methylpiperidin-4-yl)oxy)phenyl)piperidin-1-yl)-2-oxoacetamide (Compound 58)

Prepared by general procedure scheme 7.1 step 5A. Yield: 26.7 mg (16.05%).

HPLC conditions: Column: SunFire C18 100*19 mm, 5 microM; 2-10 min 25-50% water-MeOH+NH₃30 ml/min; (loading pump 4 ml/min MeOH+NH₃).

Compound 58: ¹H NMR (600 MHz, DMSO-d₆) δ (ppm) 0.77-1.02 (m, 3H), 1.08-1.15 (m, 3H), 1.27-1.39 (m, 1H), 1.54-1.66 (m, 3H), 1.83-1.94 (m, 3H), 1.94-2.08 (m, 1H), 2.10-2.19 (m, 6H), 2.37-2.44 (m, 2H), 2.55-2.61 (m, 2H), 2.72-3.20 (m, 1H), 3.52-4.05 (m, 1H), 4.27-4.41 (m, 1H), 5.09-5.57 (m, 1H), 5.57-5.71 (m, 2H), 6.76-6.91 (m, 3H), 7.21-7.31 (m, 1H), 7.43-7.55 (m, 1H), 7.93-8.10 (m, 1H), 10.44-10.59 (m, 1H).

LCMS(ESI): [M]⁺ m/z: calcd 479.2; found 480.2; Rt=2.268 min.

### Example 79. Compound 113 5-(2-((2R,5S)-2-(3-cyano-2-(1-methylpiperidin-4-yl)quinolin-7-yl)-5-methylpiperidin-1-yl)-2-oxoacetamido)-2-methoxynicotinamide

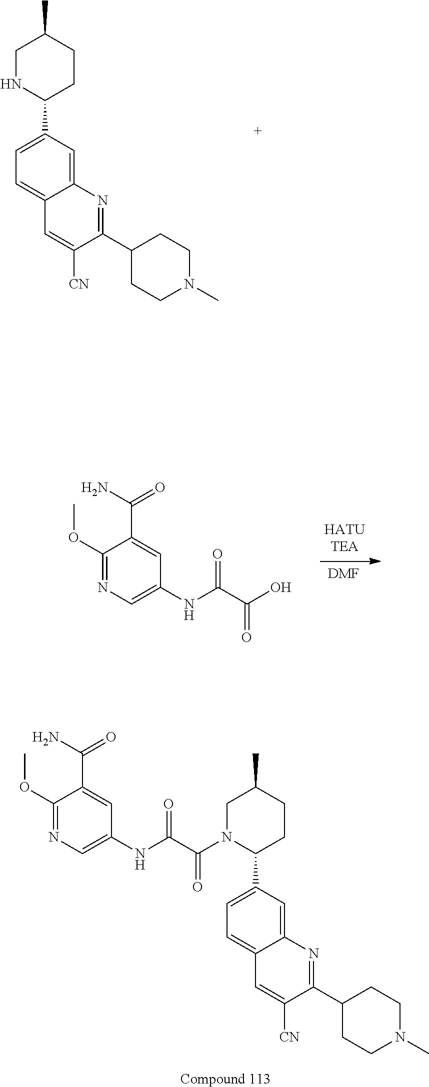

The synthesis of 7-((2R,5S)-5-methylpiperidin-2-yl)-2-(1-methylpiperidin-4-yl)quinoline-3-carbonitrile is given by the following steps 1-9.

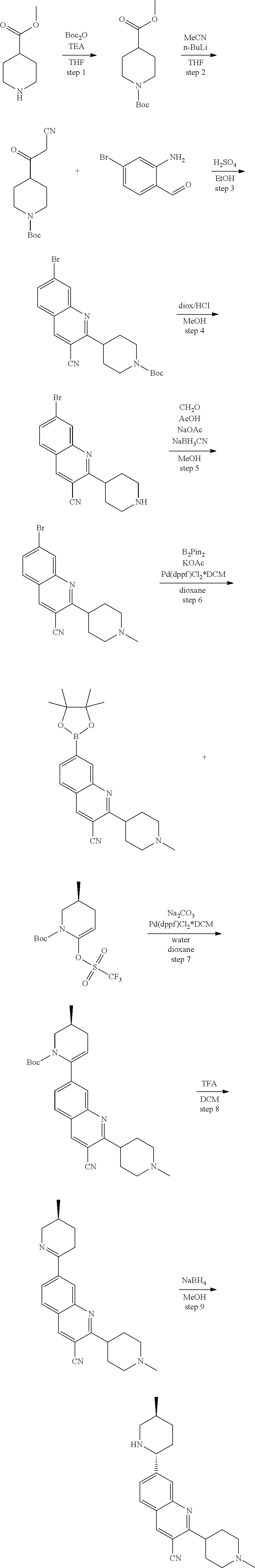

### Step 1: Synthesis of 1-tert-butyl 4-methyl piperidine-1,4-dicarboxylate

To a stirring solution of methyl piperidine-4-carboxylate (15 g, 104.76 mmol, 14.15 mL) in THF (200 mL) was added di-tert-butyl dicarbonate (25.15 g, 115.24 mmol, 26.45 mL), TEA (13.78 g, 136.19 mmol, 18.98 mL) at 0° C. The resulting mixture was stirred at 0° C. for 12 hr, and then evaporated in vacuum. The residue was diluted with water (100 mL) and extracted with DCM (2*100 mL). The combined organic extracts were dried over sodium sulphate and evaporated in vacuum to afford 1-tert-butyl 4-methyl piperidine-1,4-dicarboxylate (22 g, 90.42 mmol, 86.31% yield).

LCMS(ESI): [M-t-Bu]⁺ m/z: calcd 187.2; found 188.2; Rt=3.461 min.

### Step 2: Synthesis of tert-butyl 4-(2-cyanoacetyl)piperidine-1-carboxylate

To a stirring solution of MeCN, 99% (607.43 mg, 14.80 mmol, 772.81 μL) in THF (25 mL) at −78° C. was added n-butyllithium, 2.2M in hexane, packaged under Ar in resalable ChemSeal bottles (947.80 mg, 14.80 mmol, 6 mL) dropwise under argon. The resulting mixture was stirred at −78° C. for 3 hr, and then was added 1-tert-butyl 4-methyl piperidine-1,4-dicarboxylate (3 g, 12.33 mmol) dropwise at −78° C. stirred for 2 hr. The reaction mixture was then stirred for 3 hr at rt, then was added 10% solution NH₄Cl, and was evaporated in vacuum. The residue was diluted with water (100 mL) and extracted with EtOAc (2*30 mL). The combined organic extracts were dried over sodium sulphate and evaporated in vacuum to afford tert-butyl 4-(2-cyanoacetyl)piperidine-1-carboxylate (1.7 g, 6.74 mmol, 54.64% yield).

LCMS(ESI): [M-Boc]⁺ m/z: calcd 152.2; found 153.2; Rt=2.859 min.

### Step 3: Synthesis of tert-butyl 4-(7-bromo-3-cyanoquinolin-2-yl)piperidine-1-carboxylate

tert-Butyl 4-(2-cyanoacetyl)piperidine-1-carboxylate (0.5 g, 1.98 mmol) and 2-amino-4-bromo-benzaldehyde (396.40 mg, 1.98 mmol) was dissolved in EtOH (20 mL) and sulfuric acid (1.94 mg, 19.82 mol) was added, stirred overnight for 12 hr at 78° C. The reaction mixture was cooled to rt and reaction mixture was filtered to give tert-butyl 4-(7-bromo-3-cyano-2-quinolyl)piperidine-1-carboxylate (0.5 g, 1.20 mmol, 60.610% yield).

LCMS(ESI): [M-t-Bu]⁺ m/z: calcd 360.2; found 361.2; Rt=4.904 min.

### Step 4: Synthesis of 7-bromo-2-(piperidin-4-yl)quinoline-3-carbonitrile

Hydrogen chloride solution 4.0M in dioxane (800.00 mg, 21.94 mmol, 1 mL) was added to a solution of tert-butyl 4-(7-bromo-3-cyano-2-quinolyl)piperidine-1-carboxylate (0.5 g, 1.20 mmol) in MeOH (10 mL). The reaction mixture was stirred at 20° C. for 8 hr, then evaporated and added to MTBE (10 ml) the resulting precipitate was filtered off, washed with MTBE (10 ml) and dried to afford 7-bromo-2-(4-piperidyl)quinoline-3-carbonitrile (0.25 g, 642.48 μmol, 53.49% yield, 2HCl).

LCMS(ESI): [M]⁺ m/z: calcd 316.2; found 317.2; Rt=2.052 min.

### Step 5: Synthesis of 7-bromo-2-(1-methylpiperidin-4-yl)quinoline-3-carbonitrile

Formaldehyde, 37% w/w aq. soln., stab. with 7-8% MeOH (509.35 mg, 16.96 mmol, 470.32 μL) and acetic acid (679.02 mg, 11.31 mmol, 647.31 μL) were added to the solution of 7-bromo-2-(4-piperidyl)quinoline-3-carbonitrile (2.2 g, 5.65 mmol, 2HCl) and sodium acetate, anhydrous (1.39 g, 16.96 mmol, 910.57 μL) in MeOH (20 mL). Resulting mixture was stirred at 0° C. for 1 hr before sodium cyan borohydride (710.57 mg, 11.31 mmol) was added thereto. After that, stirring was continued for 5 hr. Then, solvent was removed under reduced pressure and residue was partitioned between 10% aq. K₂CO₃ solution (40 ml) and DCM (50 ml). Organic layer was separated, dried over solid K₂CO₃ and concentrated under reduced pressure, leaving 7-bromo-2-(1-methyl-4-piperidyl)quinoline-3-carbonitrile (2.1 g, crude).

LCMS(ESI): [M]⁺ m/z: calcd 330.2; found 331.2; Rt=0.831 min.

### Step 6: Synthesis of 2-(1-methylpiperidin-4-yl)-7-(4,4,5,5-tetramethyl-1,3,2-dioxaborolan-2-yl)quinoline-3-carbonitrile

Prepared by general procedure scheme 7.1 step 1. Yield: 2.5 g of crude.

LCMS(ESI): [M]⁺ m/z: calcd 377.2; found 378.2; Rt=1.061 min.

### Step 7: Synthesis of (S)-tert-butyl 6-(3-cyano-2-(1-methylpiperidin-4-yl)quinolin-7-yl)-3-methyl-3,4-dihydropyridine-1(2H)-carboxylate

Prepared by general procedure scheme 7.1 step 2. Yield: 3.4 g of crude.

LCMS(ESI): [M]⁺ m/z: calcd 446.2; found 447.2; Rt=2.992 min.

### Step 8: Synthesis of (S)-7-(5-methyl-3,4,5,6-tetrahydropyridin-2-yl)-2-(1-methylpiperidin-4-yl)quinoline-3-carbonitrile

Prepared by general procedure scheme 7.1 step 3. Yield: 2.2 g of crude.

LCMS(ESI): [M]⁺ m/z: calcd 346.2; found 347.2; Rt=0.567 min.

### Step 9: Synthesis of 7-((2R,5S)-5-methylpiperidin-2-yl)-2-(1-methylpiperidin-4-yl)quinoline-3-carbonitrile

Prepared by general procedure scheme 7.1 step 4. Yield: 0.7 g of crude.

LCMS(ESI): [M]⁺ m/z: calcd 348.2; found 349.2; Rt=1.864 min.

Prepared by general procedure scheme 7.1 step 5A. Yield: 177 mg (54.14%).

HPLC conditions: Column: XBridge C18 100*19 mm, 5 microM; 0-1-6 min 35-35-80% water-MeOH+0.1% NH₄OH; (loading pump 4 ml/min MeOH).

### Compound 113

¹H NMR (600 MHz, DMSO-d₆) δ (ppm) 1.04-1.04 (m, 4H), 1.35-2.34 (m, 12H), 2.93-3.12 (m, 4H), 3.36-4.33 (m, 5H), 5.40-5.76 (m, 1H), 7.64-7.75 (m, 3H), 7.91 (s, 1H), 8.04-8.09 (m, 1H), 8.37-8.58 (m, 2H), 8.97-9.01 (m, 1H), 11.01-11.17 (m, 1H).

LCMS(ESI): [M]⁺ m/z: calcd 569.2; found 570.2; Rt=2.280 min.

### Example 80. Compound 126 2-methoxy-5-(2-((2R,5S)-5-methyl-2-(2-(4-methylpiperazin-1-yl)quinolin-7-yl)piperidin-1-yl)-2-oxoacetamido)nicotinamide

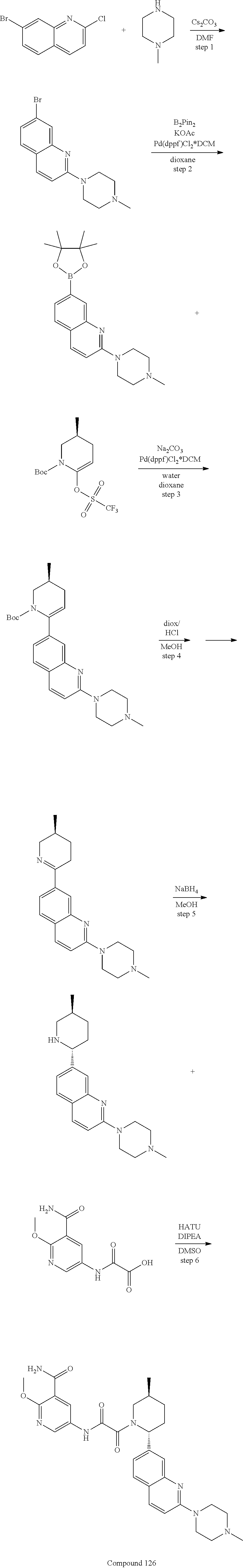

### Step 1: Synthesis of 7-bromo-2-(4-methylpiperazin-1-yl)quinoline

To a stirred solution of 7-bromo-2-chloro-quinoline (0.8 g, 3.30 mmol) and 1-methylpiperazine (396.52 mg, 3.96 mmol, 439.11 μL) in DMF (4.56 mL) was added cesium carbonate (2.15 g, 6.60 mmol), and the resulting mixture was stirred at 100° C. for 16 hr under argon atmosphere. The reaction mixture was diluted with EtOAc, and concentrated under vacuum to obtain 7-bromo-2-(4-methylpiperazin-1-yl)quinoline (0.958 g, 3.13 mmol, 94.84% yield).

LCMS(ESI): [M]⁺ m/z: calcd 305.2; found 306.2; Rt=0.835 min.

### Step 2: Synthesis of 2-(4-methylpiperazin-1-yl)-7-(4,4,5,5-tetramethyl-1,3,2-dioxaborolan-2-yl)quinoline

Prepared by general procedure scheme 7.1 step 1. Yield: 0.6 g of crude.

LCMS(ESI): [M]⁺ m/z: calcd 353.2; found 354.2; Rt=0.934 min.

### Step 3: Synthesis of (S)-tert-butyl 3-methyl-6-(2-(4-methylpiperazin-1-yl)quinolin-7-yl)-3,4-dihydropyridine-1(2H)-carboxylate

Prepared by general procedure scheme 7.1 step 2. Yield: 0.72 g of crude.

LCMS(ESI): [M]⁺ m/z: calcd 422.2; found 423.2; Rt=0.979 min.

### Step 4: Synthesis of (S)-7-(5-methyl-3,4,5,6-tetrahydropyridin-2-yl)-2-(4-methylpiperazin-1-yl)quinoline

tert-Butyl (3S)-3-methyl-6-[2-(4-methylpiperazin-1-yl)-7-quinolyl]-3,4-dihydro-2H-pyridine-1-carboxylate (200.00 mg, 473.30 mol) was dissolved in MeOH (1 mL) and diox/HCl (9.47 mmol, 1 mL) was added thereto. Then it was stirred at rt for 2 hr. The reaction mixture was evaporated to afford 2-(4-methylpiperazin-1-yl)-7-[(3S)-3-methyl-2,3,4,5-tetrahydropyridin-6-yl]quinoline (0.16 g, 496.21 μmol, 104.84% yield).

LCMS(ESI): [M]⁺ m/z: calcd 322.2; found 323.2; Rt=0.700 min.

### Step 5: Synthesis of 2-(4-methylpiperazin-1-yl)-7-((2R,5S)-5-methylpiperidin-2-yl)quinoline

Prepared by general procedure scheme 7.1 step 4. Yield: 0.14 g of crude.

LCMS(ESI): [M]⁺ m/z: calcd 324.2; found 325.2; Rt=0.696 min.

### Step 6: Synthesis of 2-methoxy-5-(2-((2R,5S)-5-methyl-2-(2-(4-methylpiperazin-1-yl)quinolin-7-yl)piperidin-1-yl)-2-oxoacetamido)nicotinamide (Compound 126)

Prepared by general procedure scheme 7.1 step 5B. Yield: 17.4 mg (7.39%).

HPLC conditions: Column: SunFire C18 100*19 mm, 5 microM; 2-10 min 25-50% MeOH+NH₃30 ml/min; (loading pump 4 ml/min MeOH+NH₃).

Compound 126: ¹H NMR (600 MHz, DMSO-d₆) δ (ppm) 0.73-1.10 (m, 3H), 1.27-1.45 (m, 1H), 1.65-1.78 (m, 1H), 1.83-1.98 (m, 1H), 2.02-2.20 (m, 1H), 2.21 (s, 3H), 2.26-2.36 (m, 1H), 2.39-2.44 (m, 4H), 2.79-3.27 (m, 1H), 3.50-3.57 (m, 0.7H), 3.62-3.70 (m, 4H), 3.88-4.02 (m, 3H), 4.04-4.09 (m, 0.3H), 5.20-5.82 (m, 1H), 7.15-7.27 (m, 2H), 7.43-7.50 (m, 1H), 7.66-7.80 (m, 3H), 7.95-8.04 (m, 1H), 8.39-8.49 (m, 1H), 8.49-8.63 (m, 1H), 10.90-11.23 (m, 1H).

LCMS(ESI): [M]⁺ m/z: calcd 504.2; found 505.2; Rt=2.176 min.

### Example 81. Compound 53 N-(6-amino-5-ethylpyridin-3-yl)-2-((2R,5S)-5-methyl-2-(2-(1-methylpiperidin-4-yl)benzo[d]oxazol-5-yl)piperidin-1-yl)-2-oxoacetamide

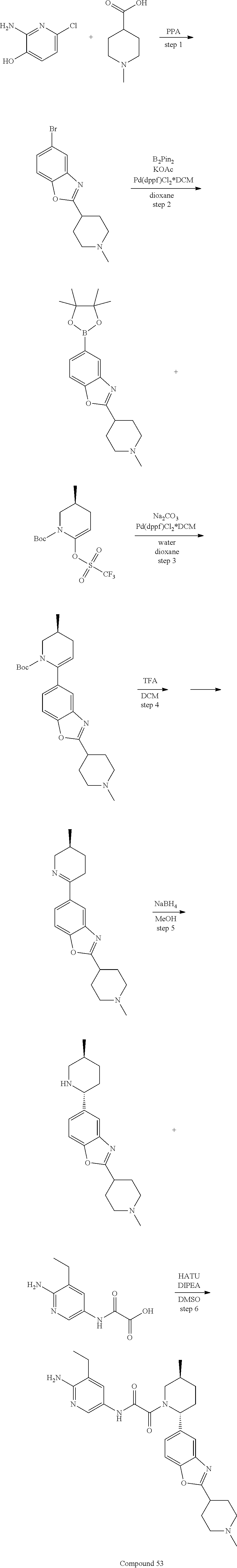

### Step 1: Synthesis of 5-bromo-2-(1-methylpiperidin-4-yl)benzo[d]oxazole

To PPA (5 g, 10.64 mmol) were added 2-amino-4-bromo-phenol (2 g, 10.64 mmol) and 1-methylpiperidine-4-carboxylic acid (1.91 g, 10.64 mmol, HCl). The mixture was stirred at 180° C. for 3.5 hr. Then it was cooled to 0° C. and 10M aqueous NaOH was added dropwise until the pH reached 8-10. To the mixture EtOAc was added and it was filtered through celite.

The filtrate was extracted, washed with water and brine, organic layer was collected, dried over Na₂SO₄ and evaporated to obtain 5-bromo-2-(1-methyl-4-piperidyl)-1,3-benzoxazole (0.82 g, crude).

LCMS(ESI): [M]⁺ m/z: calcd 295.2; found 296.2; Rt=1.000 min.

### Step 2: Synthesis of 2-(1-methylpiperidin-4-yl)-5-(4,4,5,5-tetramethyl-1,3,2-dioxaborolan-2-yl)benzo[d]oxazole

Prepared by general procedure scheme 7.1 step 1. Yield: 1.6 g of crude.

LCMS(ESI): [M]⁺ m/z: calcd 342.2; found 343.2; Rt=1.065 min.

### Step 3: Synthesis of (S)-tert-butyl 3-methyl-6-(2-(1-methylpiperidin-4-yl)benzo[d]oxazol-5-yl)-3,4-dihydropyridine-1(2H)-carboxylate)

Prepared by general procedure scheme 7.1 step 2. Yield: 1.37 g of crude.

LCMS(ESI): [M]⁺ m/z: calcd 411.2; found 412.2; Rt=0.927 min.

### Step 4: Synthesis of (S)-5-(5-methyl-3,4,5,6-tetrahydropyridin-2-yl)-2-(1-methylpiperidin-4-yl)benzo[d]oxazole

Prepared by general procedure scheme 7.1 step 3. Yield: 50 mg of crude.

LCMS(ESI): [M]⁺ m/z: calcd 311.2; found 312.2; Rt=0.489 min.

### Step 5: Synthesis of 5-((2R,5S)-5-methylpiperidin-2-yl)-2-(1-methylpiperidin-4-yl)benzo[d]oxazole

Prepared by general procedure scheme 7.1 step 4. Yield: 50 mg of crude.

LCMS(ESI): [M]⁺ m/z: calcd 313.2; found 314.2; Rt=0.478 min.

### Step 6: Synthesis of N-(6-amino-5-ethylpyridin-3-yl)-2-((2R,5S)-5-methyl-2-(2-(1-methylpiperidin-4-yl)benzo[d]oxazol-5-yl)piperidin-1-yl)-2-oxoacetamide (Compound 53)

Prepared by general procedure scheme 7.1 step 5B. Yield: 4.6 mg (5.71%).

HPLC conditions: Column: SunFire C18 100*19 mm, 5 microM; 2-10 min 55-70% MeOH+NH₃30 ml/min; (loading pump 4 ml/min MeOH).

Compound 53: ¹H NMR (600 MHz, DMSO-d₆) δ (ppm) 1.02 (m, 3H), 1.12 (m, 3H), 1.34 (dd, 1H), 1.68 (m, 1H), 1.81 (m, 3H), 2.06 (m, 5H), 2.18 (s, 3H), 2.78 (m, 2H), 2.95 (m, 2H), 3.46 (m, 1H), 4.12 (m, 1H), 5.62 (m, 3H), 7.34 (m, 2H), 7.55 (m, 1H), 7.66 (m, 2H), 8.08 (m, 2H), 10.53 (m, 1H).

LCMS(ESI): [M]⁺ m/z: calcd 504.2; found 505.2; Rt=2.176 min.

### Example 82. Compound 9 N-(6-amino-5-ethylpyridin-3-yl)-2-((2R,5S)-2-(3-(4-(dimethylamino)piperidin-1-yl)phenyl)-5-methylpiperidin-1-yl)-2-oxoacetamide

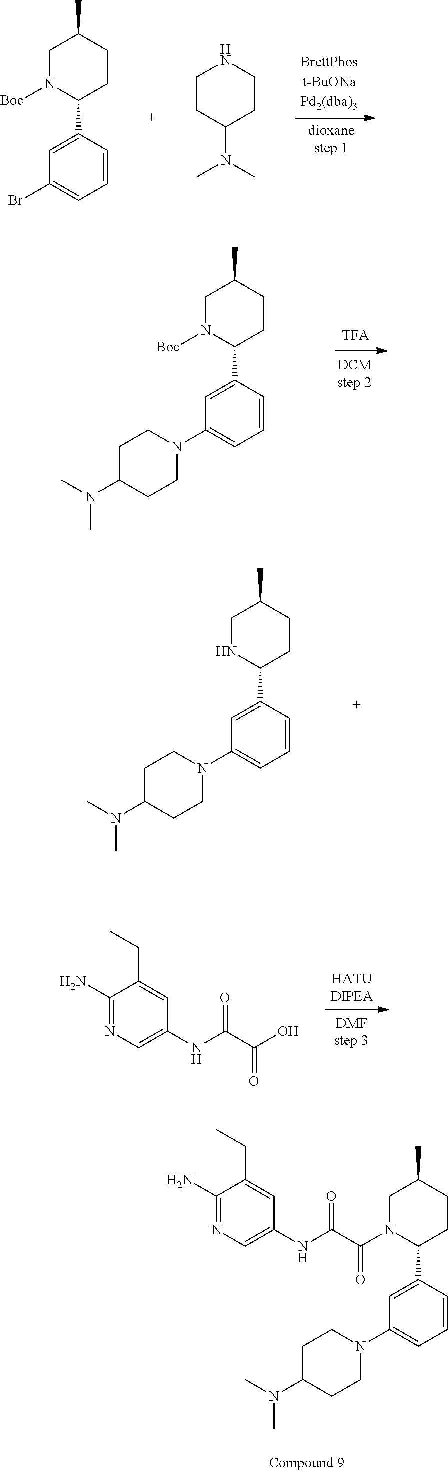

### Step 1: Synthesis of (2R,5S)-tert-butyl 2-(3-(4-(dimethylamino)piperidin-1-yl)phenyl)-5-methylpiperidine-1-carboxylate

tert-Butyl (2R,5S)-2-(3-bromophenyl)-5-methyl-piperidine-1-carboxylate (0.6 g, 1.69 mmol), N,N-dimethylpiperidin-4-amine (217.14 mg, 1.69 mmol), sodium tert-butoxide (325.52 mg, 3.39 mmol) and BrettPhos (90.91 mg, 169.36 μmol) were mixed together in dioxane (18 mL) and the resulting mixture was evacuated and backfilled three times with argon. tris(Dibenzylideneacetone)dipalladium (0) (77.54 mg, 84.68 mol) was added to the previous mixture and the resulting mixture was heated at 100° C. (oil bath) overnight. The reaction mixture was cooled and filtered; the filtrate was concentrated in vacuum. The obtained product was used in the next step without further purification. tert-Butyl (2R,5S)-2-[3-[4-(dimethylamino)-1-piperidyl]phenyl]-5-methyl-piperidine-1-carboxylate (0.77 g, crude) was obtained as a brown gum.

LCMS(ESI): [M]⁺ m/z: calcd 401.2; found 402.2; Rt=1.224 min.

### Step 2: Synthesis of N,N-dimethyl-1-(3-((2R,5S)-5-methylpiperidin-2-yl)phenyl)piperidin-4-amine

tert-Butyl (2R,5S)-2-[3-[4-(dimethylamino)-1-piperidyl]phenyl]-5-methyl-piperidine-1-carboxylate (0.77 g, 1.92 mmol) was dissolved in a mixture of DCM (7 mL) and TFA (7 mL). The resulting clear solution was stirred for 0.5 hr at 20° C. The reaction mixture was concentrated on vacuum. The obtained residue was dissolved in water and extracted with MTBE (3 times), after that the aqueous layer was basified with NaOH and extracted with DCM (3 times), combined DCM layers were dried over Na₂SO₄, filtered and evaporated. The obtained product was used in the next step without further purification. N,N-Dimethyl-1-[3-[(2R,5S)-5-methyl-2-piperidyl]phenyl]piperidin-4-amine (0.2 g, 663.42 μmol, 34.60% yield) was obtained as a brown oil.

LCMS(ESI): [M]⁺ m/z: calcd 301.2; found 302.2; Rt=0.380 min.

### Step 3: Synthesis of N-(6-amino-5-ethylpyridin-3-yl)-2-((2R,5S)-2-(3-(4-(dimethylamino)piperidin-1-yl)phenyl)-5-methylpiperidin-1-yl)-2-oxoacetamide (Compound 9)

Prepared by general procedure scheme 7.1 step 5B. Yield: 11 mg (3.08%).

### HPLC Conditions:

1-st run: Column: SunFire C18 100*19 mm, 5 microM; 2-10 min 30-80% MeOH+NH₃30 ml/min; (loading pump 4 ml/min MeOH).

2-nd run: Column: SunFire C18 100*19 mm, 5 microM; 2-10 min 0-50% MeOH+FA 30 ml/min; (loading pump 4 ml/min MeOH).

Compound 9: ¹H NMR (600 MHz, DMSO-d₆) δ (ppm) 0.77-1.02 (m, 3H), 1.06-1.13 (m, 3H), 1.27-1.38 (m, 1H), 1.44-1.53 (m, 2H), 1.59-1.72 (m, 1H), 1.77-1.88 (m, 3H), 1.90-2.03 (m, 1H), 2.04-2.21 (m, 2H), 2.23-2.28 (m, 6H), 2.38-2.43 (m, 2H), 2.60-2.77 (m, 2H), 3.42-3.46 (m, 1H), 3.62-3.75 (m, 2H), 4.01 (s, 1H), 5.03-5.56 (m, 1H), 5.56-5.73 (m, 2H), 6.66-6.77 (m, 1H), 6.79-6.88 (m, 2H), 7.14-7.24 (m, 1H), 7.42-7.52 (m, 1H), 8.00-8.11 (m, 1H), 10.40-10.62 (m, 1H).

LCMS(ESI): [M]⁺ m/z: calcd 492.2; found 493.2; Rt=1.936 min.

### Example 83. Compound 123 N-(6-amino-5-ethylpyridin-3-yl)-2-((2R,5S)-5-methyl-2-(7-(1-methylpiperidin-4-yl)naphthalen-2-yl)piperidin-1-yl)-2-oxoacetamide

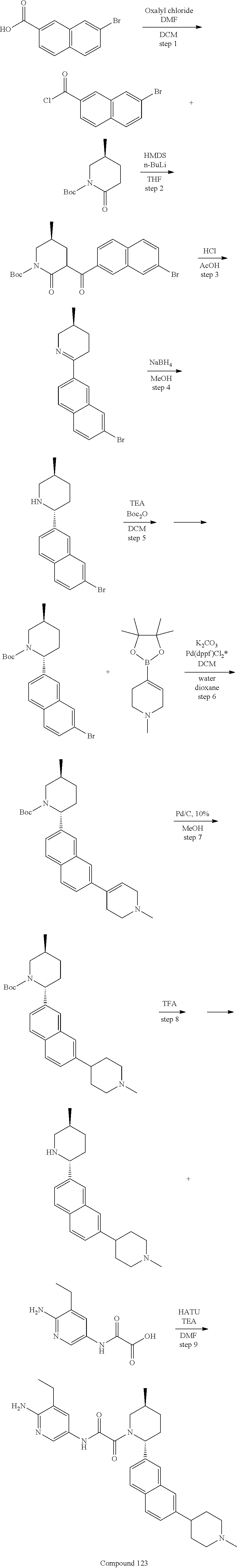

### Step 1: Synthesis of 7-bromo-2-naphthoyl chloride

Oxalyl chloride (3.43 g, 27.00 mmol, 2.36 mL) was added dropwise to a suspension of 7-bromonaphthalene-2-carboxylic acid (5.65 g, 22.50 mmol, 3.45 mL) and DMF (16.45 mg, 225.03 mol, 17.42 μL) in DCM (44.18 mL). Resulting mixture was stirred for 3 hr at 25° C. The solvent was evaporated to obtain 7-bromonaphthalene-2-carbonyl chloride (6.2 g, crude).

¹H NMR (400 MHz, CDCl₃) δ (ppm) 7.73 (m, 2H), 7.85 (m, 1H), 8.03 (m, 1H), 8.14 (m, 1H), 8.61 (m, 1H).

### Step 2: Synthesis of (5S)-tert-butyl 3-(7-bromo-2-naphthoyl)-5-methyl-2-oxopiperidine-1-carboxylate

At −78° C., n-butyllithium (1.57 g, 24.49 mmol, 9.80 mL) was added dropwise to a solution of HMDS (4.13 g, 25.60 mmol, 5.34 mL) in THF. The resulting mixture was stirred at −78° C. for 1 hr. Then the solution of tert-butyl (5S)-5-methyl-2-oxo-piperidine-1-carboxylate (4.75 g, 22.26 mmol) in THF was added at −78° C. to the previous mixture. After 30 min 7-bromonaphthalene-2-carbonyl chloride (6 g, 22.26 mmol) was added at −78° C. and the solution was allowed warm to rt and stirred overnight. The reaction mixture was quenched with NaHSO₄ (2 g; 10% solution) and extracted with DCM (3*50 ml). Organic layers were washed with water, dried over Na₂SO₄. DCM was evaporated to give tert-butyl (5S)-3-(7-bromonaphthalene-2-carbonyl)-5-methyl-2-oxo-piperidine-1-carboxylate (12.3 g, crude).

LCMS(ESI): [M-Boc]⁺ m/z: calcd 345.2; found 346.2; Rt=1.659 min.

### Step 3: Synthesis of (S)-6-(7-bromonaphthalen-2-yl)-3-methyl-2,3,4,5-tetrahydropyridine

Solution of tert-butyl (5S)-3-(7-bromonaphthalene-2-carbonyl)-5-methyl-2-oxo-piperidine-1-carboxylate (12.3 g, 27.56 mmol) in HCl (36%) (60 mL)/acetic acid (60 mL) was stirred at 100° C. for 18 hr. Upon completion, the reaction mixture was cooled. Then reaction mixture was washed with MTBE (100 ml) twice, aqueous phase was basified with KOH. Then aqueous phase was extracted with DCM 200 mL twice. The combined organic phase was washed with water 100 mL, dried over Na₂SO₄ and concentrated under reduced pressure to obtain crude product. The desired product (3S)-6-(7-bromo-2-naphthyl)-3-methyl-2,3,4,5-tetrahydropyridine (0.94 g, crude) was isolated.

LCMS(ESI): [M]⁺ m/z: calcd 302.2; found 303.2; Rt=1.073 min.

### Step 4: Synthesis of (2R,5S)-2-(7-bromonaphthalen-2-yl)-5-methylpiperidine

Sodium borohydride (176.51 mg, 4.67 mmol, 164.35 μL) was added in one portion at 0° C. to a stirred solution of (3S)-6-(7-bromo-2-naphthyl)-3-methyl-2,3,4,5-tetrahydropyridine (0.94 g, 3.11 mmol) in MeOH (10 mL). The reaction mixture was stirred at rt for 2 hr, and then concentrated in vacuum. The residue was diluted with water (10 ml) and extracted with DCM (2*15 ml). The combined organic extracts were dried over sodium sulfate and concentrated in vacuum to afford crude (2R,5S)-2-(7-bromo-2-naphthyl)-5-methyl-piperidine (0.92 g, 3.02 mmol, 97.22% yield) as brown gum, which was used directly in the next step.

LCMS(ESI): [M]⁺ m/z: calcd 304.2; found 305.2; Rt=0.780 min.

### Step 5: Synthesis of (2R,5S)-tert-butyl 2-(7-bromonaphthalen-2-yl)-5-methylpiperidine-1-carboxylate

Di-tert-butyl dicarbonate (726.00 mg, 3.33 mmol, 763.40 μL) was added dropwise to a solution of (2R,5S)-2-(7-bromo-2-naphthyl)-5-methyl-piperidine (0.92 g, 3.02 mmol) and TEA (459.01 mg, 4.54 mmol, 632.25 μL) in DCM (10 mL). Resulting mixture was stirred at rt for 3 hr. Resulting mixture was washed with water (2×10 ml), dried over Na₂SO₄, filtered and evaporated to obtain tert-butyl (2R,5S)-2-(7-bromo-2-naphthyl)-5-methyl-piperidine-1-carboxylate (1.16 g, crude).

LCMS(ESI): [M-Boc]⁺ m/z: calcd 304.2; found 305.2; Rt=1.867 min.

### Step 6: Synthesis of (2R,5S)-tert-butyl 5-methyl-2-(7-(1-methyl-1,2,3,6-tetrahydropyridin-4-yl)naphthalen-2-yl)piperidine-1-carboxylate

tert-Butyl (2R,5S)-2-(7-bromo-2-naphthyl)-5-methyl-piperidine-1-carboxylate (0.82 g, 2.03 mmol), 1-methyl-4-(4,4,5,5-tetramethyl-1,3,2-dioxaborolan-2-yl)-3,6-dihydro-2H-pyridine (475.11 mg, 2.13 mmol) and potassium carbonate-granular (840.87 mg, 6.08 mmol, 367.19 μL) were added to a mixture of dioxane (25 mL) and water (8 mL). The resulting mixture was evacuated and then backfilled with argon, this operation was repeated three times, then Pd(dppf)Cl₂*DCM (82.81 mg, 101.40 mol) was added under argon. The reaction mixture was stirred under argon at 90° C. for 18 hr, then cooled and filtered. The filter cake was washed with dioxane (2*20 ml) and discarded. The filtrate was evaporated in vacuum and diluted with water 70 ml and filtered. The filter cake was washed with EtOAc (2*20 ml). Then precipitate was dissolved in MeOH, filtered and evaporated to afford crude product that was purified by FCC to obtain (tert-butyl (2R,5S)-5-methyl-2-[7-(1-methyl-3,6-dihydro-2H-pyridin-4-yl)-2-naphthyl]piperidine-1-carboxylate (220 mg, 523.08 mol, 25.79% yield).

LCMS(ESI): [M]⁺ m/z: calcd 420.2; found 421.2; Rt=1.372 min.

### Step 7: Synthesis of (2R,5S)-tert-butyl 5-methyl-2-(7-(1-methylpiperidin-4-yl)naphthalen-2-yl)piperidine-1-carboxylate

tert-Butyl (2R,5S)-5-methyl-2-[7-(1-methyl-3,6-dihydro-2H-pyridin-4-yl)-2-naphthyl]piperidine-1-carboxylate (220 mg, 523.08 mol) was dissolved in MeOH (4 mL) and palladium, 10% on carbon, Type 487, dry (11.13 mg, 104.62 mol) was added thereto. The resulting mixture was evacuated and backfilled three times with hydrogen and the resulting mixture was hydrogenated at 1 atm (balloon) overnight. The catalyst was filtered off and the filtrate was concentrated in vacuum to obtain tert-butyl (2R,5S)-5-methyl-2-[7-(1-methyl-4-piperidyl)-2-naphthyl]piperidine-1-carboxylate (207 mg, 489.82 mol, 93.64% yield).

LCMS(ESI): [M]⁺ m/z: calcd 422.2; found 423.2; Rt=1.182 min.

### Step 8: Synthesis of 1-methyl-4-(7-((2R,5S)-5-methylpiperidin-2-yl)naphthalen-2-yl)piperidine

A solution of tert-butyl (2R,5S)-5-methyl-2-[7-(1-methyl-4-piperidyl)-2-naphthyl]piperidine-1-carboxylate (207 mg, 489.82 mol) in TFA (2 mL) was stirred at rt for 3 hr. The solvent was evaporated to dryness to obtain 1-methyl-4-[7-[(2R,5S)-5-methyl-2-piperidyl]-2-naphthyl]piperidine (308 mg, crude, 2CF₃COOH).

LCMS(ESI): [M]⁺ m/z: calcd 322.2; found 323.2; Rt=0.790 min.

### Step 9: Synthesis of N-(6-amino-5-ethylpyridin-3-yl)-2-((2R,5S)-5-methyl-2-(7-(1-methylpiperidin-4-yl)naphthalen-2-yl)piperidin-1-yl)-2-oxoacetamide (Compound 123)

Prepared by general procedure scheme 7.1 step 5A. Yield: 38 mg (15.79%).

HPLC conditions: Column: SunFire C18 100*19 mm, 5 microM; 2-10 min 30-80% MeOH+NH₃30 ml/min; (loading pump 4 ml/min MeOH).

Compound 123: ¹H NMR (600 MHz, DMSO-d₆) δ (ppm) 0.73-1.13 (m, 6H), 1.32-1.44 (m, 1H), 1.71-1.78 (m, 3H), 1.79-1.83 (m, 2H), 1.84-1.93 (m, 1H), 1.95-2.02 (m, 2H), 2.02-2.18 (m, 1H), 2.20 (s, 3H), 2.31-2.34 (m, 1H), 2.37-2.44 (m, 2H), 2.59-2.64 (m, 1H), 2.77-2.85 (m, 0.3H), 2.86-2.92 (m, 2H), 3.24-3.27 (m, 0.7H), 3.44-4.12 (m, 1H), 5.27-5.60 (m, 1H), 5.59-5.79 (m, 2H), 7.35-7.54 (m, 3H), 7.70-7.80 (m, 2H), 7.81-7.90 (m, 2H), 7.95-8.12 (m, 1H), 10.48-10.62 (m, 1H).

LCMS(ESI): [M]⁺ m/z: calcd 513.2; found 514.2; Rt=0.847 min.

### Example 84. Compound 66 N-(6-amino-5-ethylpyridin-3-yl)-2-((2R,5S)-2-(3-((S)-2-(dimethylamino)propoxy)phenyl)-5-methylpiperidin-1-yl)-2-oxoacetamide

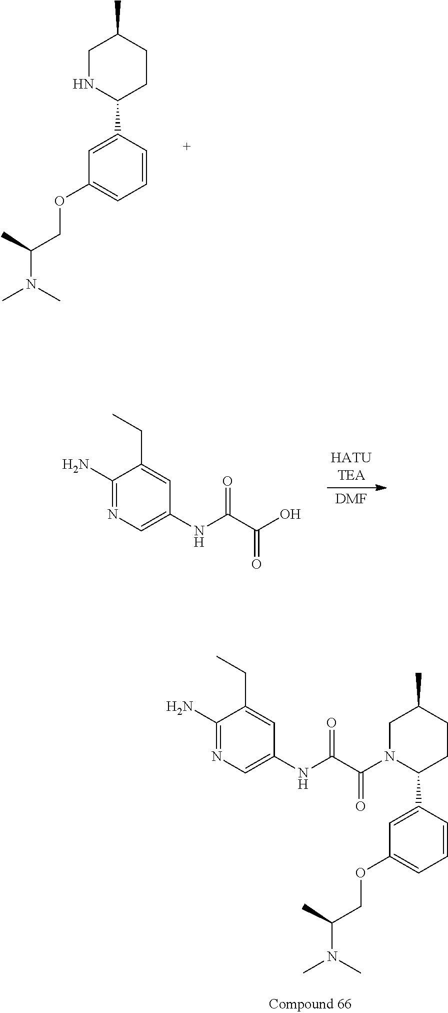

The synthesis of (S)—N,N-dimethyl-1-(3-((2R,5S)-5-methylpiperidin-2-yl)phenoxy)propan-2-amine is given by 3SSS.

Prepared by general procedure scheme 7.1 step 5A. Yield: 48.4 mg (47.29%).

HPLC conditions: Column: SunFire C18 100*19 mm, 5 microM; 2-10 min 30-60% MeOH+NH₃; (loading pump 4 ml/min MeOH).

Compound 66: ¹H NMR (600 MHz, DMSO-d₆) δ (ppm) 0.97-1.03 (m, 6H), 1.06-1.14 (m, 3H), 1.27-1.38 (m, 1H), 1.58-1.73 (m, 1H), 1.80-1.94 (m, 1H), 1.96-2.13 (m, 1H), 2.14-2.36 (m, 8H), 2.38-2.42 (m, 1H), 2.72-3.23 (m, 2H), 3.44-4.06 (m, 3H), 5.09-5.56 (m, 1H), 5.58-5.69 (m, 2H), 6.80-6.93 (m, 3H), 7.23-7.31 (m, 1H), 7.42-7.53 (m, 1H), 7.97-8.08 (m, 1H), 10.48-10.56 (m, 1H).

LCMS(ESI): [M]⁺ m/z: calcd 467.2; found 468.2; Rt=2.139 min.

### Example 85. Compound 63 N-(6-amino-5-ethylpyridin-3-yl)-2-((2R,5S)-2-(3-cyano-2-(1-methylpiperidin-4-yl)quinolin-7-yl)-5-methylpiperidin-1-yl)-2-oxoacetamide

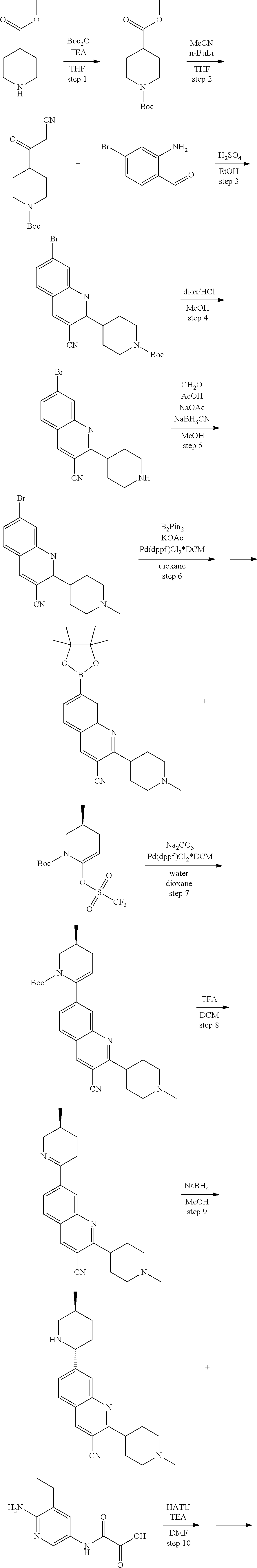

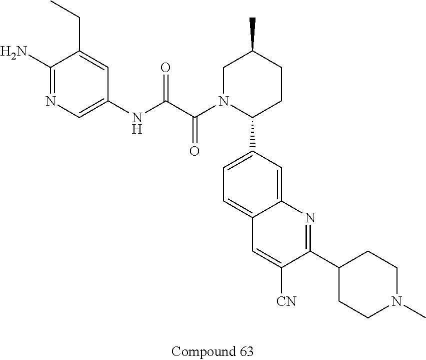 [SMILES: CCc1cc(NC(=O)C(=O)N2C[C@@H](C)CC[C@@H]2c2ccc3cc(C#N)c(C4CCN(C)CC4)nc3c2)cnc1N]

### Step 1: Synthesis of 1-tert-butyl 4-methyl piperidine-1,4-dicarboxylate

To a stirring solution of methyl piperidine-4-carboxylate (15 g, 104.76 mmol, 14.15 mL) in THF (200 mL) was added di-tert-butyl dicarbonate (25.15 g, 115.24 mmol, 26.45 mL), TEA (13.78 g, 136.19 mmol, 18.98 mL) at 0° C. The resulting mixture was stirred at 0° C. for 12 hr, and then evaporated in vacuum. The residue was diluted with water (100 mL) and extracted with DCM (2*100 mL). The combined organic extracts were dried over sodium sulphate and evaporated in vacuum to afford 1-tert-butyl 4-methyl piperidine-1,4-dicarboxylate (22 g, 90.42 mmol, 86.31% yield).

LCMS(ESI): [M-t-Bu]⁺ m/z: calcd 187.2; found 188.2; Rt=3.461 min.

### Step 2: Synthesis of tert-butyl 4-(2-cyanoacetyl)piperidine-1-carboxylate

To a stirring solution of MeCN, 99% (607.43 mg, 14.80 mmol, 772.81 μL) in THF (25 mL) at −78° C. was added n-butyllithium, 2.2M in hexane, packaged under Ar in resalable ChemSeal bottles (947.80 mg, 14.80 mmol, 6 mL) dropwise under argon. The resulting mixture was stirred at −78° C. for 3 hr, and then was added 1-tert-butyl 4-methyl piperidine-1,4-dicarboxylate (3 g, 12.33 mmol) dropwise at −78° C. stirred for 2 hr. The reaction mixture was then stirred for 3 hr at rt, then was added 10% solution NH₄Cl, and was evaporated in vacuum. The residue was diluted with water (100 mL) and extracted with EtOAc (2*30 mL). The combined organic extracts were dried over sodium sulphate and evaporated in vacuum to afford tert-butyl 4-(2-cyanoacetyl)piperidine-1-carboxylate (1.7 g, 6.74 mmol, 54.64% yield).

LCMS(ESI): [M-Boc]⁺ m/z: calcd 152.2; found 153.2; Rt=2.859 min.

### Step 3: Synthesis of tert-butyl 4-(7-bromo-3-cyanoquinolin-2-yl)piperidine-1-carboxylate

tert-Butyl 4-(2-cyanoacetyl)piperidine-1-carboxylate (0.5 g, 1.98 mmol) and 2-amino-4-bromo-benzaldehyde (396.40 mg, 1.98 mmol) was dissolved in EtOH (20 mL) and sulfuric acid (1.94 mg, 19.82 mol) was added, stirred overnight for 12 hr at 78° C. The reaction mixture was cooled to rt and reaction mixture was filtered to give tert-butyl 4-(7-bromo-3-cyano-2-quinolyl)piperidine-1-carboxylate (0.5 g, 1.20 mmol, 60.610% yield).

LCMS(ESI): [M-t-Bu]⁺ m/z: calcd 360.2; found 361.2; Rt=4.904 min.

### Step 4: Synthesis of 7-bromo-2-(piperidin-4-yl)quinoline-3-carbonitrile

Hydrogen chloride solution 4.0M in dioxane (800.00 mg, 21.94 mmol, 1 mL) was added to a solution of tert-butyl 4-(7-bromo-3-cyano-2-quinolyl)piperidine-1-carboxylate (0.5 g, 1.20 mmol) in MeOH (10 mL). The reaction mixture was stirred at 20° C. for 8 hr, then evaporated and added to MTBE (10 ml) the resulting precipitate was filtered off, washed with MTBE (10 ml) and dried to afford 7-bromo-2-(4-piperidyl)quinoline-3-carbonitrile (0.25 g, 642.48 mol, 53.49% yield, 2HCl).

LCMS(ESI): [M]⁺ m/z: calcd 316.2; found 317.2; Rt=2.052 min.

### Step 5: Synthesis of 7-bromo-2-(1-methylpiperidin-4-yl)quinoline-3-carbonitrile

Formaldehyde, 37% w/w aq. soln., stab. with 7-8% MeOH (509.35 mg, 16.96 mmol, 470.32 μL) and acetic acid (679.02 mg, 11.31 mmol, 647.31 μL) were added to the solution of 7-bromo-2-(4-piperidyl)quinoline-3-carbonitrile (2.2 g, 5.65 mmol, 2HCl) and sodium acetate, anhydrous (1.39 g, 16.96 mmol, 910.57 μL) in MeOH (20 mL). Resulting mixture was stirred at 0° C. for 1 hr before sodium cyan borohydride (710.57 mg, 11.31 mmol) was added thereto. After that, stirring was continued for 5 hr. Then, solvent was removed under reduced pressure and residue was partitioned between 10% aq. K₂CO₃ solution (40 ml) and DCM (50 ml). Organic layer was separated, dried over solid K₂CO₃ and concentrated under reduced pressure, leaving 7-bromo-2-(1-methyl-4-piperidyl)quinoline-3-carbonitrile (2.1 g, crude).

LCMS(ESI): [M]⁺ m/z: calcd 330.2; found 331.2; Rt=0.831 min.

### Step 6: Synthesis of 2-(1-methylpiperidin-4-yl)-7-(4,4,5,5-tetramethyl-1,3,2-dioxaborolan-2-yl)quinoline-3-carbonitrile

Prepared by general procedure scheme 7.1 step 1. Yield: 2.5 g of crude.

LCMS(ESI): [M]⁺ m/z: calcd 377.2; found 378.2; Rt=1.061 min.

### Step 7: Synthesis of (S)-tert-butyl 6-(3-cyano-2-(1-methylpiperidin-4-yl)quinolin-7-yl)-3-methyl-3,4-dihydropyridine-1(2H)-carboxylate

Prepared by general procedure scheme 7.1 step 2. Yield: 3.4 g of crude.

LCMS(ESI): [M]⁺ m/z: calcd 446.2; found 447.2; Rt=2.992 min.

### Step 8: Synthesis of (S)-7-(5-methyl-3,4,5,6-tetrahydropyridin-2-yl)-2-(1-methylpiperidin-4-yl)quinoline-3-carbonitrile

Prepared by general procedure scheme 7.1 step 3. Yield: 2.2 g of crude.

LCMS(ESI): [M]⁺ m/z: calcd 346.2; found 347.2; Rt=0.567 min.

### Step 9: Synthesis of 7-((2R,5S)-5-methylpiperidin-2-yl)-2-(1-methylpiperidin-4-yl)quinoline-3-carbonitrile

Prepared by general procedure scheme 7.1 step 4. Yield: 0.7 g of crude.

LCMS(ESI): [M]⁺ m/z: calcd 348.2; found 349.2; Rt=1.864 min.

### Step 10: Synthesis of N-(6-amino-5-ethylpyridin-3-yl)-2-((2R,5S)-2-(3-cyano-2-(1-methylpiperidin-4-yl)quinolin-7-yl)-5-methylpiperidin-1-yl)-2-oxoacetamide (Compound 63)

Prepared by general procedure scheme 7.1 step 5A. Yield: 46.6 mg (7.52%).

HPLC conditions: Column: YMC Triart C18 100*20 mm, 5 microM; 0-5 min 35-50% water-MeCN+0.1% NH₄OH 30 ml/min; (loading pump 4 ml/min MeCN).

Compound 63: ¹H NMR (600 MHz, DMSO-d₆) δ (ppm) 1.08 (m, 6H), 1.38 (m, 1H), 1.69 (m, 1H), 1.90 (m, 3H), 1.99 (m, 2H), 2.13 (m, 3H), 2.26 (m, 3H), 2.33 (m, 1H), 2.40 (m, 2H), 2.91 (m, 3H), 3.14 (m, 1H), 3.92 (m, 1H), 5.66 (m, 3H), 7.46 (m, 1H), 7.68 (m, 1H), 7.91 (m, 1H), 8.08 (m, 2H), 9.01 (m, 1H), 10.57 (m, 1H).

LCMS(ESI): [M]⁺ m/z: calcd 539.2; found 540.2; Rt=2.216 min.

### Example 86. Compound 49 5-(2-((2R,5S)-2-(3-((R)-2-(dimethylamino)propoxy)phenyl)-5-methylpiperidin-1-yl)-2-oxoacetamido)-2-methoxynicotinamide

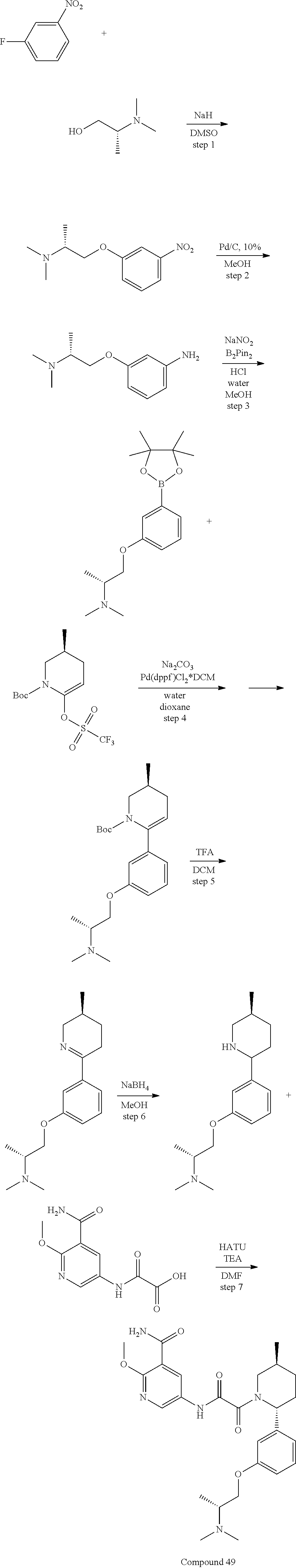

### Step 1: Synthesis of (R)—N,N-dimethyl-1-(3-nitrophenoxy)propan-2-amine

Sodium hydride (in oil dispersion) 60% dispersion in mineral oil (3.70 g, 96.67 mmol, 60% purity) was added to DMSO (90 mL) and the resulting mixture was stirred for 30 min. A solution of (2R)-2-(dimethylamino)propan-1-ol (9.07 g, 87.88 mmol) in DMSO (10 mL) was added dropwise to the previous mixture and the resulting mixture was stirred for 30 min. 1-Fluoro-3-nitro-benzene (12.4 g, 87.88 mmol, 9.36 mL) was added dropwise to the previous mixture and the resulting mixture was stirred for 1.5 hr. The reaction mixture was quenched with aq·NH₄Cl (100 ml) and the resulting mixture was extracted with EtOAc (3*100 ml). Combined organic layers were washed with brine (3*80 ml), dried over Na₂SO₄, filtered and concentrated in vacuum. The residue was purified by flash chromatography (gradient MeOH in MTBE from 0% to 50%) to obtain (2R)—N,N-dimethyl-1-(3-nitrophenoxy)propan-2-amine (13.7 g, 61.09 mmol, 69.52% yield).

LCMS(ESI): [M]⁺ m/z: calcd 224.2; found 225.2; Rt=0.486 min.

### Step 2: Synthesis of (R)-3-(2-(dimethylamino)propoxy)aniline

(2R)—N,N-Dimethyl-1-(3-nitrophenoxy)propan-2-amine (13.7 g, 61.09 mmol) was dissolved in MeOH (280 mL) and palladium, 10% on carbon, Type 487, dry (1.30 g, 12.22 mmol) was added thereto. The resulting mixture was evacuated and backfilled three times with hydrogen. The reaction mixture was hydrogenated at 1 atm (balloon) overnight. 0.5 g of 10% Pd/C was added to the reaction mixture and the resulting mixture was evacuated and backfilled three times with hydrogen. The reaction mixture was hydrogenated at 1 atm (balloon) overnight. The catalyst was filtered off and the filtrate was concentrated in vacuum to obtain 3-[(2R)-2-(dimethylamino)propoxy]aniline (11.44 g, crude).

LCMS(ESI): [M]⁺ m/z: calcd 194.2; found 195.2; Rt=0.155 min.

### Step 3: Synthesis of (R)—N,N-dimethyl-1-(3-(4,4,5,5-tetramethyl-1,3,2-dioxaborolan-2-yl)phenoxy)propan-2-amine

3-[(2R)-2-(Dimethylamino)propoxy]aniline (11.4 g, 58.68 mmol) was dissolved in MeOH (125 mL) and hydrochloric acid, 36% w/w aq. soln. (17.83 g, 176.04 mmol, 14.86 mL, 36% purity) was added thereto followed by the addition of water (100 mL). The resulting mixture was cooled to 0° C. in an ice bath and a solution of sodium nitrite (4.45 g, 64.55 mmol, 2.05 mL) in water (25 mL) was added dropwise at 0° C. After addition completed, the resulting mixture was stirred for 30 min. A solution of bis(pinacolato) diboron (19.37 g, 76.28 mmol) was added to the previous mixture and the resulting mixture was stirred for 14 hr. The reaction mixture was diluted with water (250 ml) and the resulting mixture was extracted with DCM (3*150 ml). The aqueous layer was basified with NaHCO₃ and extracted with DCM (3*200 ml). Combined organic layers were dried over Na₂SO₄, filtered and concentrated in vacuum to obtain (2R)—N,N-dimethyl-1-[3-(4,4,5,5-tetramethyl-1,3,2-dioxaborolan-2-yl)phenoxy]propan-2-amine (10.7 g, crude).

LCMS(ESI): [M]⁺ m/z: calcd 305.2; found 306.2; Rt=0.955 min.

### Step 4: Synthesis of (S)-tert-butyl 6-(3-((R)-2-(dimethylamino)propoxy)phenyl)-3-methyl-3,4-dihydropyridine-1(2H)-carboxylate

Prepared by general procedure scheme 7.1 step 2. Yield: 1.85 g (14.09%).

CC conditions: The crude product was purified by silica gel with MTBE/MeOH as an eluent mixture.

LCMS(ESI): [M]⁺ m/z: calcd 374.2; found 375.2; Rt=0.906 min.

### Step 5: Synthesis of (R)—N,N-dimethyl-1-(3-((S)-5-methyl-3,4,5,6-tetrahydropyridin-2-yl)phenoxy)propan-2-amine

Prepared by general procedure scheme 7.1 step 3. Yield: 0.95 g of crude.

¹H NMR (400 MHz, CDCl₃) δ (ppm) 0.96 (d, 3H), 1.21 (m, 4H), 1.36 (m, 1H), 1.71 (m, 1H), 1.87 (m, 1H), 2.42 (s, 6H), 2.58 (m, 1H), 2.74 (m, 1H), 3.08 (m, 1H), 3.24 (m, 1H), 4.05 (m, 2H), 6.92 (m, 1H), 7.26 (m, 2H), 7.37 (m, 1H).

### Step 6: Synthesis of (R)—N,N-dimethyl-1-(3-((2R,5S)-5-methylpiperidin-2-yl)phenoxy)propan-2-amine

Prepared by general procedure scheme 7.1 step 4. Yield: 850 mg of crude.

¹H NMR (500 MHz, CDCl₃) δ (ppm) 0.90 (d, 3H), 1.19 (m, 6H), 1.86 (m, 4H), 2.43 (s, 6H), 3.08 (m, 2H), 3.59 (m, 1H), 3.96 (m, 2H), 6.82 (m, 1H), 6.90 (m, 1H), 7.05 (m, 1H), 7.21 (m, 1H).

### Step 7: Synthesis of 5-(2-((2R,5S)-2-(3-((R)-2-(dimethylamino)propoxy)phenyl)-5-methylpiperidin-1-yl)-2-oxoacetamido)-2-methoxynicotinamide (Compound 49)

Prepared by general procedure scheme 7.1 step 5A. Yield: 35.1 mg (13.00%).

HPLC conditions: Column: SunFire C18 100*19 mm, 5 microM; 2-10 min 35-50% water-MeOH+NH₃30 ml/min; (loading pump 4 ml/min MeOH+NH₃).

Compound 49: ¹H NMR (600 MHz, DMSO-d₆) δ (ppm) 1.00-1.04 (m, 6H), 1.29-1.36 (m, 1H), 1.65-2.21 (m, 10H), 2.78-2.88 (m, 2H), 3.46-3.48 (m, 1H), 3.77-4.01 (m, 5H), 5.13-5.55 (m, 1H), 6.81-6.91 (m, 3H), 7.26-7.31 (m, 1H), 7.70-7.74 (m, 2H), 8.42-8.56 (m, 2H), 11.00-11.08 (m, 1H).

LCMS(ESI): [M]⁺ m/z: calcd 497.2; found 498.2; Rt=2.150 min.

### Example 87. Compound 73 N-(6-amino-5-ethylpyridin-3-yl)-2-(2-(2-(1-methylpiperidin-4-yl)benzo[d]thiazol-5-yl)piperidin-1-yl)-2-oxoacetamide

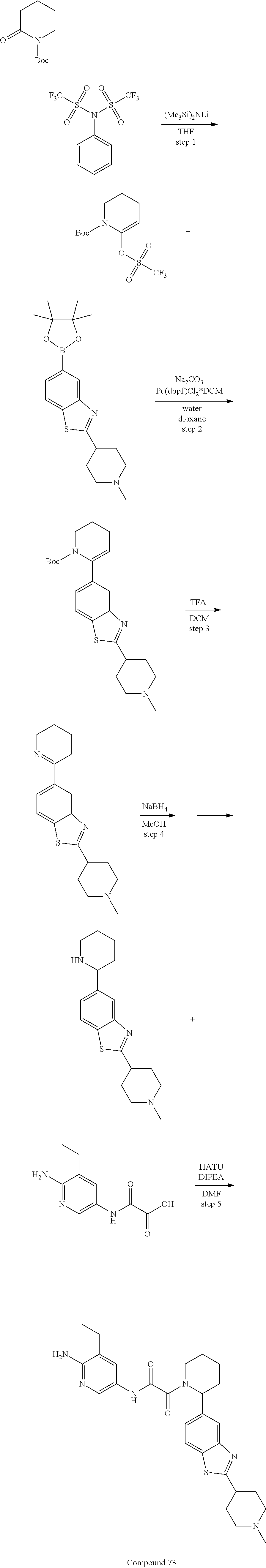

### Step 1: Synthesis of tert-butyl 6-(((trifluoromethyl)sulfonyl)oxy)-3,4-dihydropyridine-1(2H)-carboxylate

Lithium bis(trimethylsilyl)amide (9.17 g, 54.81 mmol) (1.08 M in THF/ethylbenzene) was added for 0.5 hr dropwise under argon to a cooled to −78° C. solution of tert-butyl 2-oxopiperidine-1-carboxylate (9.1 g, 45.67 mmol) in THF (100 mL). The resulting solution was stirred at −78° C. for 1.5 hr, then 1,1,1-trifluoro-N-phenyl-N-(trifluoromethylsulfonyl)methanesulfonamide (17.13 g, 47.96 mmol) was added in one portion. The reaction mixture was allowed to warm slowly (cooling bath was not removed!) to 20° C. and stirred for 12 hr. The reaction mixture was diluted with water and MTBE. The organic layer was separated; the aqueous layer was additionally extracted with MTBE. The combined organic extracts were washed with 10% aqueous sodium hydroxide solution, dried over Na₂SO₄ and concentrated in vacuum. The residue was moved to silica gel pad, washed with hexanes then with hexanes/MTBE-1/1, hexanes/MTBE was concentrated on vacuum. The obtained product was used in the next step without further purification. tert-Butyl 6-(trifluoromethylsulfonyloxy)-3,4-dihydro-2H-pyridine-1-carboxylate (9 g, 27.17 mmol, 59.48% yield) was obtained as a red liquid.

¹H NMR (500 MHz, CDCl₃) δ (ppm) 1.48 (s, 9H), 1.73 (m, 2H), 2.25 (m, 2H), 3.59 (m, 2H), 5.27 (m, 1H).

### Step 2: Synthesis of tert-butyl 6-(2-(1-methylpiperidin-4-yl)benzo[d]thiazol-5-yl)-3,4-dihydropyridine-1(2H)-carboxylate

Prepared by general procedure scheme 7.1 step 2. Yield: 1.7 g of crude.

LCMS(ESI): [M-Boc]⁺ m/z: calcd 313.2; found 314.2; Rt=0.582 min.

### Step 3: Synthesis of 2-(1-methylpiperidin-4-yl)-5-(3,4,5,6-tetrahydropyridin-2-yl)benzo[d]thiazole

Prepared by general procedure scheme 7.1 step 3. Yield: 0.9 g of crude.

LCMS(ESI): [M]⁺ m/z: calcd 313.2; found 314.2; Rt=0.186 min.

### Step 4: Synthesis of 2-(1-methylpiperidin-4-yl)-5-(piperidin-2-yl)benzo[d]thiazole

Prepared by general procedure scheme 7.1 step 4. Yield: 0.6 g of crude.

LCMS(ESI): [M]⁺ m/z: calcd 315.2; found 316.2; Rt=0.517 min.

### Step 5: Synthesis of N-(6-amino-5-ethylpyridin-3-yl)-2-(2-(2-(1-methylpiperidin-4-yl)benzo[d]thiazol-5-yl)piperidin-1-yl)-2-oxoacetamide (Compound 73)

Prepared by general procedure scheme 7.1 step 5B. Yield: 42 mg (7.99%).

HPLC conditions: Column: SunFire C18 100*19 mm, 5 microM; 2-10 min 0-50% MeOH+FA 30 ml/min; (loading pump 4 ml/min MeOH).

Compound 73: ¹H NMR (600 MHz, DMSO-d₆) δ (ppm) 1.04-1.14 (m, 3H), 1.38-1.48 (m, 1H), 1.51-1.69 (m, 3H), 1.78-1.89 (m, 3H), 1.91-2.07 (m, 1H), 2.07-2.16 (m, 4H), 2.24 (s, 3H), 2.36-2.43 (m, 2H), 2.56-2.62 (m, 1H), 2.88-2.91 (m, 2H), 3.02-3.08 (m, 1H), 3.68-3.70 (m, 0.7H), 4.28-4.31 (m, 0.3H), 5.26-5.62 (m, 1H), 5.63-5.81 (m, 2H), 7.32-7.43 (m, 1H), 7.43-7.59 (m, 1H), 7.84-7.91 (m, 1H), 7.96-8.11 (m, 2H), 10.49-10.65 (m, 1H).

LCMS(ESI): [M]⁺ m/z: calcd 506.2; found 507.2; Rt=1.874 min.

### Example 88. Compound 37 5-(2-((2R,5S)-2-(3-((S)-2-(dimethylamino)propoxy)phenyl)-5-methylpiperidin-1-yl)-2-oxoacetamido)-2-methoxynicotinamide

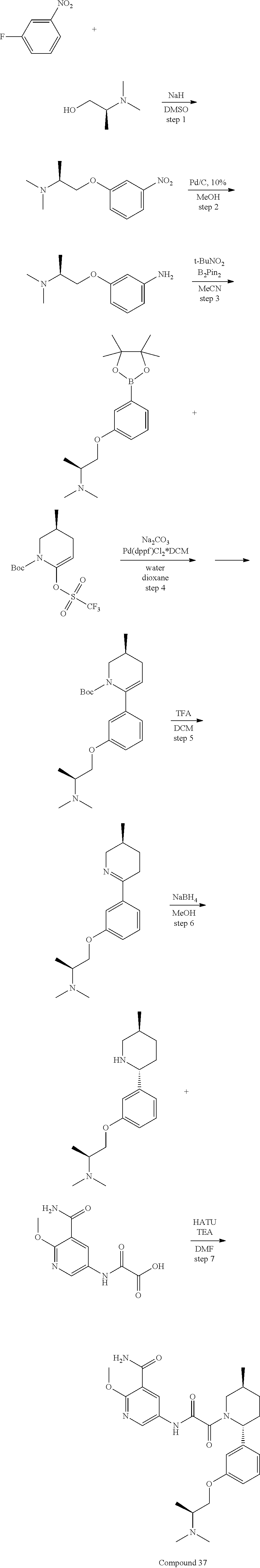

### Step 1: Synthesis of (S)—N,N-dimethyl-1-(3-nitrophenoxy)propan-2-amine

Sodium hydride (in oil dispersion) 60% dispersion in mineral oil (405.15 mg, 10.13 mmol, 60% purity) was added to DMSO (7 mL) and the resulting mixture was stirred for 30 min. A solution of (2S)-2-(dimethylamino)propan-1-ol (950 mg, 9.21 mmol) in DMSO (2.5 mL) was added dropwise to the previous mixture and the resulting mixture was stirred for 30 min. 1-Fluoro-3-nitro-benzene (1.30 g, 9.21 mmol, 980.65 μL) was added dropwise to the previous mixture and the resulting mixture was stirred for 1.5 hr. The reaction mixture was quenched with aq·NH₄Cl (50 ml) and the resulting mixture was extracted with EtOAc (3*50 ml). Combined organic layers were washed with brine (3*40 ml), dried over Na₂SO₄, filtered and concentrated in vacuum. The residue was and purified by flash chromatography (gradient MeOH in MTBE from 0% to 50%) to obtain (2S)—N,N-dimethyl-1-(3-nitrophenoxy)propan-2-amine (1.14 g, 5.07 mmol, 55.11% yield).

LCMS(ESI): [M]⁺ m/z: calcd 224.2; found 225.2; Rt=0.758 min.

### Step 2: Synthesis of (S)-3-(2-(dimethylamino)propoxy)aniline

(2S)—N,N-Dimethyl-1-(3-nitrophenoxy)propan-2-amine (1.14 g, 5.07 mmol) was dissolved in MeOH (20 mL) and palladium, 10% on carbon, Type 487, dry (270.02 mg, 2.54 mmol) was added thereto. The resulting mixture was evacuated and backfilled three times with hydrogen. The reaction mixture was hydrogenated at 1 atm (balloon) overnight. 25% of starting material has left, according to LCMS of aliquot. 200 mg of 10% Pd/C was added to the reaction mixture and the resulting mixture was evacuated and backfilled three times with hydrogen. The reaction mixture was hydrogenated at 1 atm (balloon) overnight. The catalyst was filtered off and the filtrate was concentrated in vacuum to obtain 3-[(2S)-2-(dimethylamino)propoxy]aniline (948 mg, 4.88 mmol, 96.16% yield).

LCMS(ESI): [M]⁺ m/z: calcd 194.2; found 195.2; Rt=0.203 min.

### Step 3: Synthesis of (S)—N,N-dimethyl-1-(3-(4,4,5,5-tetramethyl-1,3,2-dioxaborolan-2-yl)phenoxy)propan-2-amine

3-[(2S)-2-(Dimethylamino)propoxy]aniline (817 mg, 4.21 mmol) was dissolved in MeCN (11.25 mL) and bis(pinacolato) diboron (1.17 g, 4.63 mmol) was added thereto followed by addition of tert-butyl nitrite, tech. 90% (650.50 mg, 6.31 mmol, 750.28 μL). The resulting mixture was heated at 80° C. (oil bath) overnight. 12% of Starting material left by LCMS. Additional portions of tert-butyl nitrite, tech. 90% (650.50 mg, 6.31 mmol, 750.28 L) and bis(pinacolato) diboron (1.17 g, 4.63 mmol) was added to the reaction mixture and the resulting mixture was heated at 80° C. overnight. The reaction mixture was concentrated in vacuum. The residue was dissolved in DCM (50 ml) and the resulting solution was washed with 3% HCl solution (2*10 ml). The organic layer was dried over Na₂SO₄, filtered and concentrated in vacuum to obtain (2S)—N,N-dimethyl-1-[3-(4,4,5,5-tetramethyl-1,3,2-dioxaborolan-2-yl)phenoxy]propan-2-amine (1.62 g, crude).

LCMS(ESI): [M]⁺ m/z: calcd 305.2; found 306.2; Rt=0.901 min.

### Step 4: Synthesis of (S)-tert-butyl 6-(3-((S)-2-(dimethylamino)propoxy)phenyl)-3-methyl-3,4-dihydropyridine-1(2H)-carboxylate

Prepared by general procedure scheme 7.1 step 2. Yield: 0.16 g (8.05%).

CC conditions: The crude product was purified by silica gel with MTBE/MeOH 0-50% as an eluent mixture.

LCMS(ESI): [M]⁺ m/z: calcd 374.2; found 375.2; Rt=1.075 min.

### Step 5: Synthesis of (S)—N,N-dimethyl-1-(3-((S)-5-methyl-3,4,5,6-tetrahydropyridin-2-yl)phenoxy)propan-2-amine

Prepared by general procedure scheme 7.1 step 3. Yield: 152 mg of crude.

LCMS(ESI): [M]⁺ m/z: calcd 274.2; found 275.2; Rt=0.414 min.

### Step 6: Synthesis of (S)—N,N-dimethyl-1-(3-((2R,5S)-5-methylpiperidin-2-yl)phenoxy)propan-2-amine

Prepared by general procedure scheme 7.1 step 4. Yield: 121 mg of crude.

LCMS(ESI): [M]⁺ m/z: calcd 276.2; found 277.2; Rt=0.666 min.

### Step 7: Synthesis of 5-(2-((2R,5S)-2-(3-((S)-2-(dimethylamino)propoxy)phenyl)-5-methylpiperidin-1-yl)-2-oxoacetamido)-2-methoxynicotinamide (Compound 37)

Prepared by general procedure scheme 7.1 step 5A. Yield: 29.4 mg (27.00%).

HPLC conditions: Column: SunFire C18 100*19 mm, 5 microM; 2-10 min 35-50% water-MeOH+NH₃30 ml/min; (loading pump 4 ml/min MeOH+NH₃).

Compound 37: ¹H NMR (600 MHz, DMSO-d₆) δ (ppm) 0.98-1.06 (m, 6H), 1.27-1.38 (m, 1H), 1.61-1.73 (m, 1H), 1.82-1.95 (m, 1H), 2.00-2.14 (m, 1H), 2.16-2.25 (m, 7H), 2.77-3.16 (m, 4H), 3.46-3.82 (m, 1H), 3.92-4.05 (m, 4H), 5.10-5.58 (m, 1H), 6.81-6.85 (m, 1H), 6.86-6.94 (m, 1H), 7.24-7.33 (m, 1H), 7.65-7.78 (m, 2H), 8.41-8.48 (m, 1H), 8.49-8.58 (m, 1H), 10.95-11.15 (m, 1H).

LCMS(ESI): [M]⁺ m/z: calcd 497.2; found 498.2; Rt=2.518 min.

### Example 89. Compound 39 N-(6-amino-5-ethylpyridin-3-yl)-2-((2R,5S)-5-methyl-2-(3-((1-methylpyrrolidin-3-yl)oxy)phenyl)piperidin-1-yl)-2-oxoacetamide

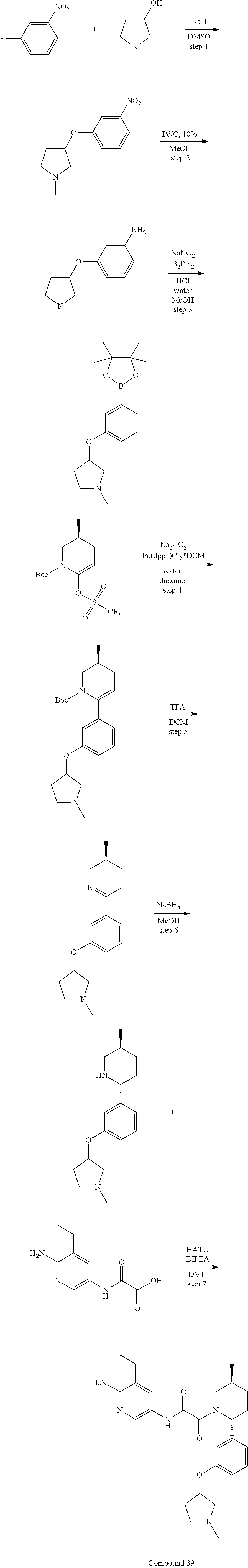

### Step 1: Synthesis of 1-methyl-3-(3-nitrophenoxy)pyrrolidine

Sodium hydride (in oil dispersion) 60% dispersion in mineral oil (1.25 g, 54.57 mmol, 20.91 μL) was added to DMSO (30 mL) and the resulting mixture was stirred for 30 min. A solution of 1-methylpyrrolidin-3-ol (5.02 g, 49.61 mmol) in DMSO (10 mL) was added dropwise to the previous mixture and the resulting mixture was stirred for 30 min. 1-Fluoro-3-nitro-benzene (7 g, 49.61 mmol, 5.28 mL) was added dropwise to the previous mixture and the resulting mixture was stirred for 1.5 hr. The reaction mixture was quenched with aq·NH₄Cl (50 ml) and the resulting mixture was extracted with EtOAc (3*50 ml). Combined organic layers were washed with brine (3*40 ml), then diluted with water and acidified with NaHSO₄, the organic layer was separated and the aqueous layer was extracted with EtOAc (2 times), after that the aqueous layer was basified with K₂CO₃ and extracted with DCM (3 times). Combined DCM layers was dried over Na₂SO₄, filtered and concentrated in vacuum. 1-Methyl-3-(3-nitrophenoxy)pyrrolidine (4 g, 18.00 mmol, 36.28% yield) was obtained as a red liquid.

LCMS(ESI): [M]⁺ m/z: calcd 222.2; found 223.2; Rt=0.731 min.

### Step 2: Synthesis of 3-((1-methylpyrrolidin-3-yl)oxy)aniline

A solution of 1-methyl-3-(3-nitrophenoxy)pyrrolidine (4 g, 18.00 mmol) in MeOH (50 mL) was hydrogenated over Pd/C (10%) (18.00 mmol) under H₂ (1 atm) at 20° C. for 12 hr. The reaction mixture was filtered, the filter cake was washed with MeOH and the filtrate was concentrated under reduced pressure. The obtained product was use in the next step without further purification. 3-(1-Methylpyrrolidin-3-yl)oxyaniline (2.5 g, 13.00 mmol, 72.25% yield) was obtained as a red oil.

LCMS(ESI): [M]⁺ m/z: calcd 192.2; found 193.2; Rt=0.154 min.

### Step 3: Synthesis of 1-methyl-3-(3-(4,4,5,5-tetramethyl-1,3,2-dioxaborolan-2-yl)phenoxy)pyrrolidine

3-(1-Methylpyrrolidin-3-yl)oxyaniline (1.5 g, 7.80 mmol) was dissolved in MeOH (15 mL)+water (15 mL)+HCl (36% aq. s-n) (3 mL), the reaction mixture was cooled to 0° C. and solution of sodium nitrite (592.14 mg, 8.58 mmol, 273.13 μL) in water (3 mL) was added dropwise at 0° C., the reaction mixture was stirred at 0° C. for 30 min then solution of bis(pinacolato) diboron (5.94 g, 23.41 mmol) in MeOH (15 mL) was added dropwise at the same temperature. The reaction mixture was stirred at 0° C. for 1 hr. The reaction mixture was diluted with water and extracted with DCM (3 times), after that the aqueous layer was basified with Na₂CO₃ and extracted with DCM (3 times). The combined DCM layers after basification was dried over Na₂SO₄, filtered and concentrated on vacuum. The obtained product was used in the next step without further purification. 1-Methyl-3-[3-(4,4,5,5-tetramethyl-1,3,2-dioxaborolan-2-yl)phenoxy]pyrrolidine (2.6 g, crude) was obtained as a red oil.

LCMS(ESI): [M]⁺ m/z: calcd 303.2; found 304.2; Rt=0.949 min.

### Step 4: Synthesis of (3S)-tert-butyl 3-methyl-6-(3-((1-methylpyrrolidin-3-yl)oxy)phenyl)-3,4-dihydropyridine-1(2H)-carboxylate

Prepared by general procedure scheme 7.1 step 2. Yield: 1.8 g of crude.

LCMS(ESI): [M]⁺ m/z: calcd 372.2; found 373.2; Rt=1.086 min.

### Step 5: Synthesis of (3S)-3-methyl-6-(3-((1-methylpyrrolidin-3-yl)oxy)phenyl)-2,3,4,5-tetrahydropyridine

Prepared by general procedure scheme 7.1 step 3. Yield: 0.18 g of crude.

LCMS(ESI): [M]⁺ m/z: calcd 272.2; found 273.2; Rt=0.591 min.

### Step 6: Synthesis of (2R,5S)-5-methyl-2-(3-((1-methylpyrrolidin-3-yl)oxy)phenyl)piperidine

Prepared by general procedure scheme 7.1 step 4. Yield: 0.13 g of crude.

LCMS(ESI): [M]⁺ m/z: calcd 274.2; found 275.2; Rt=0.520 min.

### Step 7: Synthesis of N-(6-amino-5-ethylpyridin-3-yl)-2-((2R,5S)-5-methyl-2-(3-((1-methylpyrrolidin-3-yl)oxy)phenyl)piperidin-1-yl)-2-oxoacetamide (Compound 39)

Prepared by general procedure scheme 7.1 step 5B. Yield: 11.6 mg (4.79%).

HPLC conditions: Column: SunFire C18 100*19 mm, 5 microM; 2-10 min 0-50% MeCN+FA 30 ml/min; (loading pump 4 ml/min MeCN).

Compound 39: ¹H NMR (600 MHz, DMSO-d₆) δ (ppm) 0.73-1.03 (m, 3H), 1.07-1.13 (m, 3H), 1.26-1.38 (m, 1H), 1.58-1.77 (m, 2H), 1.79-1.93 (m, 1H), 1.94-2.14 (m, 1H), 2.15-2.22 (m, 1H), 2.22-2.25 (m, 3H), 2.25-2.30 (m, 1H), 2.32-2.37 (m, 2H), 2.37-2.44 (m, 2H), 2.67-2.80 (m, 2H), 3.21-3.24 (m, 1H), 3.49-4.01 (m, 1H), 4.80-4.90 (m, 1H), 5.09-5.57 (m, 1H), 5.57-5.69 (m, 2H), 6.69-6.82 (m, 2H), 6.84-6.93 (m, 1H), 7.22-7.31 (m, 1H), 7.42-7.52 (m, 1H), 7.98-8.12 (m, 1H), 10.47-10.57 (m, 1H).

LCMS(ESI): [M]⁺ m/z: calcd 465.2; found 466.2; Rt=1.877 min.

### Example 90. Compound 92 N-(6-amino-5-ethylpyridin-3-yl)-2-((2R,5S)-5-methyl-2-(1′-methylspiro[benzo[d][1,3]dioxole-2,4′-piperidin]-5-yl)piperidin-1-yl)-2-oxoacetamide

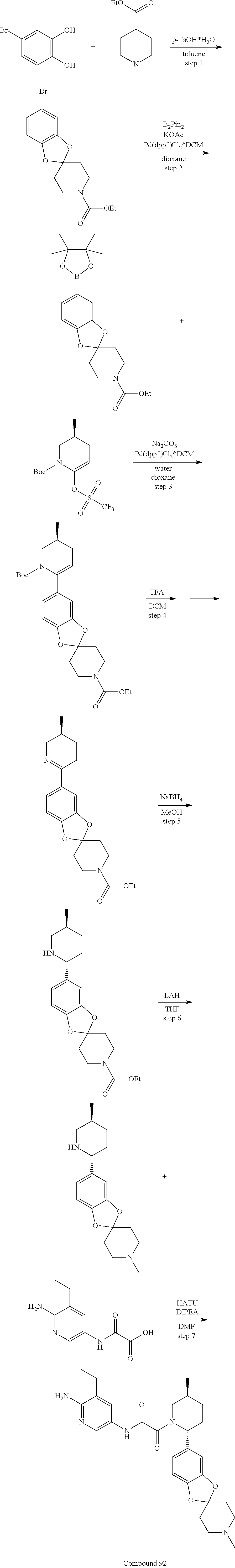

### Step 1: Synthesis of ethyl 5-bromospiro[benzo[d][1,3]dioxole-2,4′-piperidine]-1′-carboxylate

4-Bromobenzene-1,2-diol (24.8 g, 131.21 mmol) and ethyl 4-oxopiperidine-1-carboxylate (22.46 g, 131.21 mmol) were mixed in toluene (250 mL) and p-toluenesulfonic acid monohydrate (2.50 g, 13.12 mmol, 2.01 mL) was added thereto. The resulting mixture was refluxed overnight under the Dean-Stark trap. The reaction mixture was cooled, washed with aq. NaHCO₃ (2*100 ml) and concentrated in vacuum. The residue was purified by column chromatography (gradient MTBE in hexane from 0% to 100%) to obtain ethyl 5-bromospiro[1,3-benzodioxole-2,4′-piperidine]-1′-carboxylate (4.62 g, 13.51 mmol, 10.29% yield).

LCMS(ESI): [M]⁺ m/z: calcd 342.2; found 343.2; Rt=1.421 min.

### Step 2: Synthesis of ethyl 5-(4,4,5,5-tetramethyl-1,3,2-dioxaborolan-2-yl)spiro[benzo[d][1,3]dioxole-2,4′-piperidine]-1′-carboxylate

Prepared by general procedure scheme 7.1 step 1. Yield: 2.13 g of crude.

LCMS(ESI): [M]⁺ m/z: calcd 389.2; found 390.2; Rt=1.662 min.

### Step 3: Synthesis of (S)-ethyl 5-(1-(tert-butoxycarbonyl)-5-methyl-1,4,5,6-tetrahydropyridin-2-yl)spiro[benzo[d][1,3]dioxole-2,4′-piperidine]-1′-carboxylate

Prepared by general procedure scheme 7.1 step 2. Yield: 1.09 g of crude.

LCMS(ESI): [M-Boc]⁺ m/z: calcd 358.2; found 359.2; Rt=1.597 min.

### Step 4: Synthesis of (S)-ethyl 5-(5-methyl-3,4,5,6-tetrahydropyridin-2-yl)spiro[benzo[d][1,3]dioxole-2,4′-piperidine]-1′-carboxylate

Prepared by general procedure scheme 7.1 step 3. Yield: 0.982 g of crude.

LCMS(ESI): [M]⁺ m/z: calcd 358.2; found 359.2; Rt=1.047 min.

### Step 5: Synthesis of ethyl 5-((2R,5S)-5-methylpiperidin-2-yl)spiro[benzo[d][1,3]dioxole-2,4′-piperidine]-1′-carboxylate

Prepared by general procedure scheme 7.1 step 4. Yield: 0.721 g of crude.

LCMS(ESI): [M]⁺ m/z: calcd 360.2; found 361.2; Rt=1.073 min.

### Step 6: Synthesis of 1′-methyl-5-((2R,5S)-5-methylpiperidin-2-yl)spiro[benzo[d][1,3]dioxole-2,4′-piperidine]

Lithium aluminium tetrahydride (379.60 mg, 10.00 mmol) was suspended in THF (25 mL) and the resulting mixture was heated to reflux. A solution of ethyl 5-[(2R,5S)-5-methyl-2-piperidyl]spiro[1,3-benzodioxole-2,4′-piperidine]-1′-carboxylate (721 mg, 2.00 mmol) in THF (5 mL) was added dropwise to the previous mixture maintaining gentle reflux. After addition completed, the reaction mixture was refluxed overnight. The reaction mixture was cooled in an ice bath and water (0.38 ml) was carefully added dropwise followed by addition of aq·KOH solution (0.38 ml) and water (0.76 ml). After addition completed, the resulting mixture was stirred for 30 min and filtered. The filter cake was rinsed with THF and the filtrate was concentrated in vacuum to obtain 1′-methyl-5-[(2R,5S)-5-methyl-2-piperidyl]spiro[1,3-benzodioxole-2,4′-piperidine](589 mg, 1.95 mmol, 97.37% yield).

LCMS(ESI): [M]⁺ m/z: calcd 302.2; found 303.2; Rt=0.674 min.

### Step 7: Synthesis of N-(6-amino-5-ethylpyridin-3-yl)-2-((2R,5S)-5-methyl-2-(1′-methylspiro[benzo[d][1,3]dioxole-2,4′-piperidin]-5-yl)piperidin-1-yl)-2-oxoacetamide (Compound 92)

Prepared by general procedure scheme 7.1 step 5A. Yield: 127.3 mg (39.00%).

HPLC conditions: Column: SunFire C18 100*19 mm, 5 microM; 2-10 min 25-50% water-MeOH+NH₃30 ml/min; (loading pump 4 ml/min MeOH).

Compound 92: ¹H NMR (600 MHz, DMSO-d₆) δ (ppm) 0.75-1.04 (m, 3H), 1.06-1.14 (m, 3H), 1.22-1.37 (m, 1H), 1.55-1.86 (m, 2H), 1.86-1.97 (m, 5H), 1.97-2.20 (m, 2H), 2.21 (s, 3H), 2.31-2.44 (m, 3H), 2.50-2.58 (m, 2H), 2.72-3.24 (m, 1H), 3.39-3.98 (m, 1H), 4.94-5.51 (m, 1H), 5.53-5.71 (m, 2H), 6.70-6.77 (m, 1H), 6.78-6.91 (m, 2H), 7.34-7.55 (m, 1H), 7.92-8.11 (m, 1H), 10.43 (br s, 1H).

LCMS(ESI): [M]⁺ m/z: calcd 493.2; found 494.2; Rt=2.143 min.

### Example 91. Compound 76 N-(6-amino-5-ethylpyridin-3-yl)-2-((2R,5S)-5-methyl-2-(quinolin-7-yl)piperidin-1-yl)-2-oxoacetamide

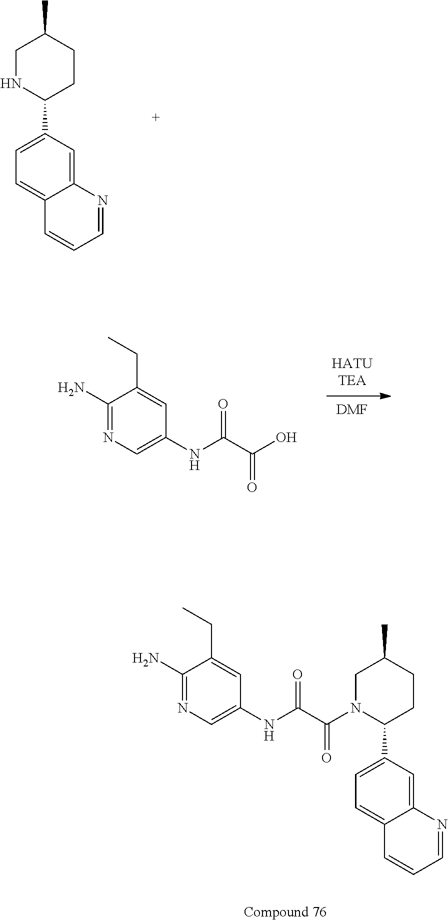

Prepared by general procedure scheme 7.1 step 5A. Yield: 161 mg (44.53%).

HPLC conditions: Column: XBridge BEH C18 100*19 mm, 5 microM; 0-1-6 min 15-15-45% water-MeCN+0.1% NH₄OH; (loading pump 4 ml/min MeCN).

Compound 76: ¹H NMR (600 MHz, DMSO-d₆) δ (ppm) 0.72-1.14 (m, 6H), 1.36-1.42 (m, 1H), 1.70-1.92 (m, 2H), 2.11-2.41 (m, 4H), 2.80-2.82 (m, 1H), 3.53-4.10 (m, 1H), 5.39-5.77 (m, 3H), 7.44-7.64 (m, 3H), 7.93-8.11 (m, 3H), 8.33-8.36 (m, 1H), 8.89 (s, 1H), 10.55-10.63 (m, 1H).

LCMS(ESI): [M]⁺ m/z: calcd 417.2; found 418.2; Rt=1.830 min.

### Example 92. Compound 26 2-methoxy-5-(2-((2R,5S)-5-methyl-2-(3-(((S)-1-methylpyrrolidin-2-yl)methoxy)phenyl)piperidin-1-yl)-2-oxoacetamido)nicotinamide

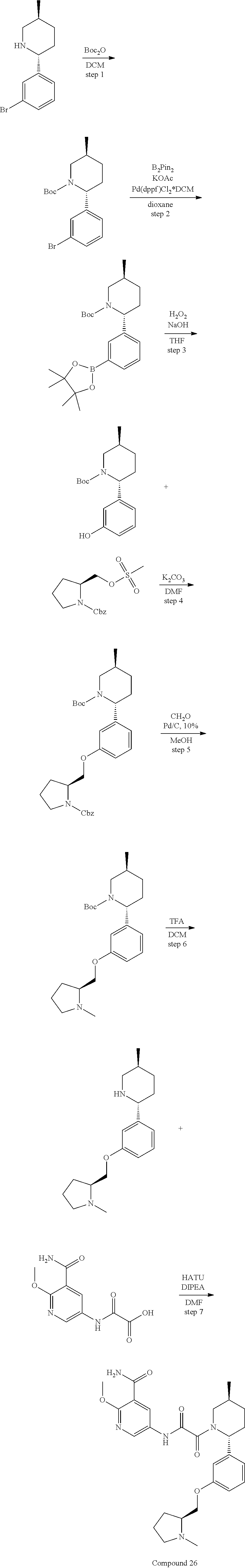

### Step 1: Synthesis of (2R,5S)-tert-butyl 2-(3-bromophenyl)-5-methylpiperidine-1-carboxylate

To a solution of (2R,5S)-2-(3-bromophenyl)-5-methyl-piperidine (3.26 g, 12.83 mmol) in DCM (30 mL) was added tert-butoxycarbonyl tert-butyl carbonate (2.80 g, 12.83 mmol, 2.94 mL) portion wise at 25C and stirred for 2 hr. The resulting solution was evaporated to dryness to give tert-butyl (2R,5S)-2-(3-bromophenyl)-5-methyl-piperidine-1-carboxylate (4.7 g, crude) as a yellow gum.

LCMS(ESI): [M-Boc]⁺ m/z: calcd 254.2; found 255.2; Rt=1.747 min.

### Step 2: Synthesis of (2R,5S)-tert-butyl 5-methyl-2-(3-(4,4,5,5-tetramethyl-1,3,2-dioxaborolan-2-yl)phenyl)piperidine-1-carboxylate

Prepared by general procedure scheme 7.1 step 1. Yield: 6.5 g of crude.

LCMS(ESI): [M-Boc]⁺ m/z: calcd 301.2; found 302.2; Rt=1.835 min.

### Step 3: Synthesis of (2R,5S)-tert-butyl 2-(3-hydroxyphenyl)-5-methylpiperidine-1-carboxylate

tert-Butyl (2R,5S)-5-methyl-2-[3-(4,4,5,5-tetramethyl-1,3,2-dioxaborolan-2-yl)phenyl]piperidine-1-carboxylate (6.5 g, 16.20 mmol) was dissolved in THF (65 mL) and hydrogen peroxide 35% (2.36 g, 24.29 mmol, 2.15 mL, 35% purity) was carefully added dropwise at rt. After addition completed, the reaction mixture was stirred for 1 hr and aq. solution of sodium hydroxide, pearl (1.04 g, 25.91 mmol, 486.63 μL) was added dropwise at rt. After addition completed, the reaction mixture was stirred for 1 hr. The reaction mixture was acidified with citric acid and the resulting mixture was transferred to a separation funnel. An organic layer was separated and the aqueous layer was extracted with MTBE (2*40 ml). Combined organic layers were washed with aq. sodium sulfite, dried over Na₂SO₄, filtered and concentrated in vacuum to obtain tert-butyl (2R,5S)-2-(3-hydroxyphenyl)-5-methyl-piperidine-1-carboxylate (6 g, crude).

LCMS(ESI): [M-t-Bu]⁺ m/z: calcd 235.2; found 236.2; Rt=1.367 min.

### Step 4: Synthesis of (2R,5S)-tert-butyl 2-(3-(((S)-1-((benzyloxy)carbonyl)pyrrolidin-2-yl)methoxy)phenyl)-5-methylpiperidine-1-carboxylate

tert-Butyl (2R,5S)-2-(3-hydroxyphenyl)-5-methyl-piperidine-1-carboxylate (2 g, 6.86 mmol), benzyl (2S)-2-(methylsulfonyloxymethyl)pyrrolidine-1-carboxylate (2.80 g, 8.92 mmol) and potassium carbonate, anhydrous, 99% (1.90 g, 13.73 mmol, 828.48 μL) was mixed together in DMF (25 mL) and heated at 81° C. for 36 hr. The reaction mixture was concentrated on vacuum. The obtained residue was dissolved in EtOAc/H₂O, the EtOAc layer was separated and the aqueous layer was extracted twice with EtOAc. The combined organic layers was washed with brine (3 times), dried over Na₂SO₄, filtered and concentrated on vacuum. The crude product was purified by FCC (MTBE in hexanes from 0% to 100%). tert-Butyl (2R,5S)-5-methyl-2-[3-[[(25)-1-benzyloxycarbonylpyrrolidin-2-yl]methoxy]phenyl]piperidine-1-carboxylate (0.55 g, 1.08 mmol, 15.75% yield) was obtained as a brown gum.

LCMS(ESI): [M-Boc]⁺ m/z: calcd 408.2; found 409.2; Rt=1.689 min.

### Step 5: Synthesis of (2R,5S)-tert-butyl 5-methyl-2-(3-(((S)-1-methylpyrrolidin-2-yl)methoxy)phenyl)piperidine-1-carboxylate

A solution of tert-butyl (2R,5S)-5-methyl-2-[3-[[(2S)-1-benzyloxycarbonylpyrrolidin-2-yl]methoxy]phenyl]piperidine-1-carboxylate (0.55 g, 1.08 mmol) and formaldehyde, 37% w/w aq. soln., stab. with 7-8% MeOH (162.34 mg, 5.41 mmol, 149.89 μL) in MeOH (16 mL) was hydrogenated under H₂ (1 atm) at 20° C. for 60 hr. The reaction mixture was filtered, the filter cake was washed with MeOH and the filtrate was concentrated under reduced pressure. The obtained product was use in the next step without further purification. tert-Butyl (2R,5S)-5-methyl-2-[3-[[(25)-1-methylpyrrolidin-2-yl]methoxy]phenyl]piperidine-1-carboxylate (0.3 g, 772.12 mol, 71.41% yield) was obtained as a brown gum.

LCMS(ESI): [M]⁺ m/z: calcd 388.2; found 389.2; Rt=1.114 min.

### Step 6: Synthesis of (2R,5S)-5-methyl-2-(3-(((S)-1-methylpyrrolidin-2-yl)methoxy)phenyl)piperidine

tert-butyl (2R,5S)-5-methyl-2-[3-[[(25)-1-methylpyrrolidin-2-yl]methoxy]phenyl]piperidine-1-carboxylate (0.3 g, 772.12 mol) was dissolved in TFA (5 mL) and DCM (5 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated. The obtained product was used in the next step without further purification. (2R,5S)-5-Methyl-2-[3-[[(2S)-1-methylpyrrolidin-2-yl]methoxy]phenyl]piperidine (0.25 g, crude) was obtained as a brown gum.

LCMS(ESI): [M]⁺ m/z: calcd 288.2; found 289.2; Rt=0.667 min.

### Step 7: Synthesis of 2-methoxy-5-(2-((2R,5S)-5-methyl-2-(3-(((S)-1-methylpyrrolidin-2-yl)methoxy)phenyl)piperidin-1-yl)-2-oxoacetamido)nicotinamide (Compound 26)

Prepared by general procedure scheme 7.1 step 5B. Yield: 5 mg (1.13%).

HPLC conditions: Column: SunFire C18 100*19 mm, 5 microM; 2-10 min 10-40% MeOH+NH₃30 ml/min; (loading pump 4 ml/min MeOH).

Compound 26: ¹H NMR (600 MHz, DMSO-d₆) δ (ppm) 0.96-1.04 (m, 3H), 1.25-1.37 (m, 1H), 1.51-1.69 (m, 3H), 1.83-2.05 (m, 3H), 2.12-2.21 (m, 2H), 2.22-2.36 (m, 3H), 2.76-2.96 (m, 3H), 3.77-3.82 (m, 1H), 3.92-3.97 (m, 3H), 3.97-4.04 (m, 1H), 5.06-5.68 (m, 1H), 6.73-6.93 (m, 3H), 7.22-7.36 (m, 1H), 7.64-7.79 (m, 2H), 8.35-8.49 (m, 1H), 8.49-8.61 (m, 1H), 8.62-8.79 (m, 1H), 9.26-9.53 (m, 1H), 10.95-11.17 (m, 1H).

LCMS(ESI): [M]⁺ m/z: calcd 509.2; found 510.2; Rt=2.621 min.

### Example 93. Compound 31 N-(6-amino-5-ethylpyridin-3-yl)-2-((2R,5S)-5-methyl-2-(1′-methyl-3H-spiro[benzofuran-2,4′-piperidin]-6-yl)piperidin-1-yl)-2-oxoacetamide

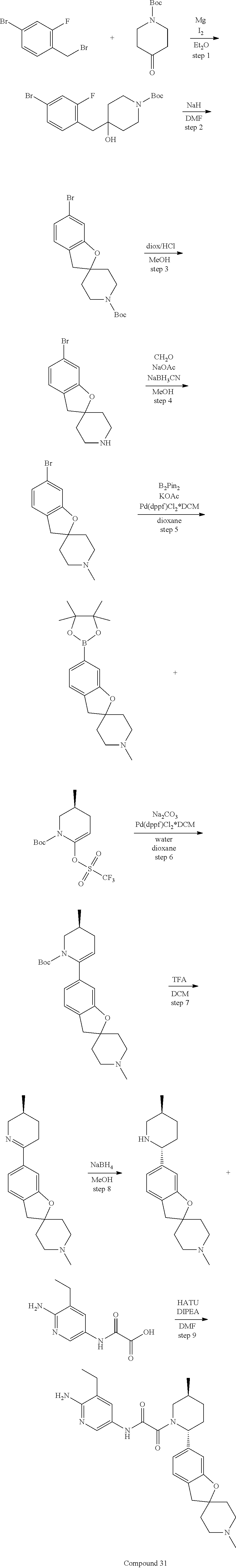

### Step 1: Synthesis of tert-butyl 4-(4-bromo-2-fluorobenzyl)-4-hydroxypiperidine-1-carboxylate

To a solution of magnesium (1.36 g, 55.99 mmol) and iodine (189.46 mg, 746.49 μmol) in Et₂O (100 mL) at reflux was added 4-bromo-1-(bromomethyl)-2-fluoro-benzene (10 g, 37.32 mmol) slowly and stirred for 30 min. To a solution of tert-butyl 4-oxopiperidine-1-carboxylate (7.44 g, 37.32 mmol) in Et₂O was added Grignard reagent at −78° C. and the reaction was stirred rt for overnight. Water and EtOAc (300 mL) was added. The organic layer was separated and dried over sodium sulfate. After concentration under reduced pressure to afford tert-butyl 4-[(4-bromo-2-fluoro-phenyl)methyl]-4-hydroxy-piperidine-1-carboxylate (12 g, 30.91 mmol, 82.80% yield).

LCMS(ESI): [M-Boc]⁺ m/z: calcd 287.2; found 288.2; Rt=1.566 min.

### Step 2: Synthesis of tert-butyl 6-bromo-3H-spiro[benzofuran-2,4′-piperidine]-1′-carboxylate

A solution of tert-butyl 4-[(4-bromo-2-fluoro-phenyl)methyl]-4-hydroxy-piperidine-1-carboxylate (12 g, 30.91 mmol) in DMF (75 mL) was added dropwise to a suspension of sodium hydride (in oil dispersion) 60% dispersion in mineral oil (1.42 g, 61.81 mmol) in DMF. The reaction mixture was heated to reflux overnight. After cooling to rt, it was evaporated and DCM and water was added. The organic layer was washed with brine (200 mL), dried over Na₂SO₄, filtered and concentrated to yield 20 g of crude product as oil. The product was purified by silica gel column chromatography (CHCl₃/MTBE) to give tert-butyl 6-bromospiro[3H-benzofuran-2,4′-piperidine]-1′-carboxylate (1.5 g, 4.07 mmol, 13.18% yield).

LCMS(ESI): [M-Boc]⁺ m/z: calcd 267.2; found 268.2; Rt=1.575 min.

### Step 3: Synthesis of 6-bromo-3H-spiro[benzofuran-2,4′-piperidine]

tert-Butyl 6-bromospiro[3H-benzofuran-2,4′-piperidine]-1′-carboxylate (1.5 g, 4.07 mmol) was dissolved in MeOH (20 mL) and diox/HCl (4.07 mmol, 5 mL) was added and stirred at rt for 2 hr. Then it was evaporated to afford 6-bromospiro[3H-benzofuran-2,4′-piperidine](0.9 g, 2.95 mmol, 72.54% yield, HCl).

LCMS(ESI): [M]⁺ m/z: calcd 267.2; found 268.2; Rt=0.887 min.

### Step 4: Synthesis of 6-bromo-1′-methyl-3H-spiro[benzofuran-2,4′-piperidine]

Formaldehyde, 37% w/w aq. soln., stab. with 7-8% MeOH (177.43 mg, 5.91 mmol, 163.83 μL) and sodium acetate (484.75 mg, 5.91 mmol, 317.25 μL) were added to the solution of 6-bromospiro[3H-benzofuran-2,4′-piperidine](0.9 g, 2.95 mmol, HCl) in MeOH (49.52 mL) Resulting mixture was stirred at 25° C. for 1 hr before sodium cyan borohydride (371.35 mg, 5.91 mmol) was added thereto. After that, stirring was continued for 16 hr. Then, solvent was removed under reduced pressure and residue was partitioned between 15% aq. K₂CO₃ solution (30 ml) and DCM (50 ml). Organic layer was separated, dried over solid Na₂SO₄ and concentrated under reduced pressure, leaving 6-bromo-1′-methyl-spiro[3H-benzofuran-2,4′-piperidine](0.8 g, 2.84 mmol, 95.96% yield).

LCMS(ESI): [M]⁺ m/z: calcd 282.2; found 283.2; Rt=0.904 min.

### Step 5: Synthesis of 1′-methyl-6-(4,4,5,5-tetramethyl-1,3,2-dioxaborolan-2-yl)-3H-spiro[benzofuran-2,4′-piperidine]

Prepared by general procedure scheme 7.1 step 1. Yield: 0.9 g of crude.

LCMS(ESI): [M]⁺ m/z: calcd 329.2; found 330.2; Rt=1.047 min.

### Step 6: Synthesis of (S)-tert-butyl 3-methyl-6-(1′-methyl-3H-spiro[benzofuran-2,4′-piperidin]-6-yl)-3,4-dihydropyridine-1(2H)-carboxylate

Prepared by general procedure scheme 7.1 step 2. Yield: 1 g of crude.

LCMS(ESI): [M]⁺ m/z: calcd 398.2; found 399.2; Rt=1.140 min.

### Step 7: Synthesis of (S)-1′-methyl-6-(5-methyl-3,4,5,6-tetrahydropyridin-2-yl)-3H-spiro[benzofuran-2,4′-piperidine]

Prepared by general procedure scheme 7.1 step 3. Yield: 0.6 g of crude.

LCMS(ESI): [M]⁺ m/z: calcd 298.2; found 299.2; Rt=0.513 min.

### Step 8: Synthesis of 1′-methyl-6-((2R,5S)-5-methylpiperidin-2-yl)-3H-spiro[benzofuran-2,4′-piperidine]

Prepared by general procedure scheme 7.1 step 4. Yield: 0.3 g of crude.

LCMS(ESI): [M]⁺ m/z: calcd 300.2; found 301.2; Rt=0.667 min.

### Step 9: Synthesis of N-(6-amino-5-ethylpyridin-3-yl)-2-((2R,5S)-5-methyl-2-(1′-methyl-3H-spiro[benzofuran-2,4′-piperidin]-6-yl)piperidin-1-yl)-2-oxoacetamide (Compound 31)

Prepared by general procedure scheme 7.1 step 5B. Yield: 23.8 mg (4.12%).

HPLC conditions: Column: SunFire C18 100*19 mm, 5 microM; 2-10 min 25-50% MeCN+FA 30 ml/min; (loading pump 4 ml/min MeCN).

Compound 31: ¹H NMR (600 MHz, DMSO-d₆) δ (ppm) 0.98-1.04 (m, 3H), 1.07-1.13 (m, 3H), 1.28-1.36 (m, 1H), 1.66-1.73 (m, 1H), 1.82-1.92 (m, 1H), 1.97-2.11 (m, 2H), 2.17 (s, 3H), 2.25-2.36 (m, 7H), 2.38-2.42 (m, 3H), 2.74-3.15 (m, 1H), 3.93-4.08 (m, 1H), 5.06-5.56 (m, 1H), 5.57-5.67 (m, 2H), 6.14-6.20 (m, 1H), 6.65-6.75 (m, 1H), 6.75-6.84 (m, 1H), 7.00-7.05 (m, 1H), 7.45-7.53 (m, 1H), 8.00-8.09 (m, 1H), 9.22-9.49 (m, 1H), 10.38-10.52 (m, 1H).

LCMS(ESI): [M]⁺ m/z: calcd 491.2; found 492.2; Rt=0.669 min.

### Example 94. Compound 29 2-methoxy-5-(2-(2-(2-(1-methylpiperidin-4-yl)benzo[d]thiazol-5-yl)piperidin-1-yl)-2-oxoacetamido)nicotinamide

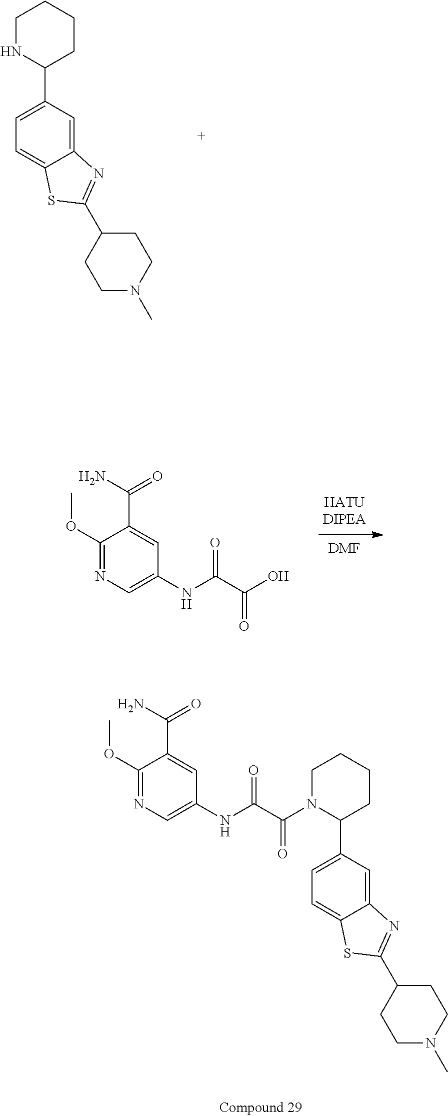

The synthesis of 2-(1-methylpiperidin-4-yl)-5-(piperidin-2-yl)benzo[d]thiazole is by general procedure scheme 7.1 step 4. Yield: 0.6 g of crude.

LCMS(ESI): [M]⁺ m/z: calcd 315.2; found 316.2; Rt=0.517 min.

Prepared by general procedure scheme 7.1 step 5B. Yield: 163 mg (31.94%).

HPLC conditions: Column: SunFire C18 100*19 mm, 5 microM; 2-10 min 35-50% water-MeOH+NH₃; (loading pump 4 ml/min MeOH).

### Compound 29

¹H NMR (600 MHz, DMSO-d₆) δ (ppm) 1.43-1.49 (m, 1H), 1.54-1.69 (m, 3H), 1.68-1.99 (m, 4H), 1.99-2.10 (m, 5H), 2.53-2.64 (m, 2H), 2.81-2.86 (m, 2H), 2.99-3.11 (m, 2H), 3.68-3.77 (m, 0.7H), 3.90-3.97 (m, 3H), 4.27-4.34 (m, 0.3H), 5.27-5.81 (m, 1H), 7.33-7.44 (m, 1H), 7.64-7.78 (m, 2H), 7.86-7.92 (m, 1H), 8.02-8.11 (m, 1H), 8.40-8.61 (m, 2H), 11.11 (s, 1H).

LCMS(ESI): [M]⁺ m/z: calcd 536.2; found 537.2; Rt=1.810 min.

### Example 95. Compound 128 N-(6-amino-5-ethylpyridin-3-yl)-2-((2R,5S)-2-(3-(2-(dimethylamino)ethoxy)-4-fluorophenyl)-5-methylpiperidin-1-yl)-2-oxoacetamide

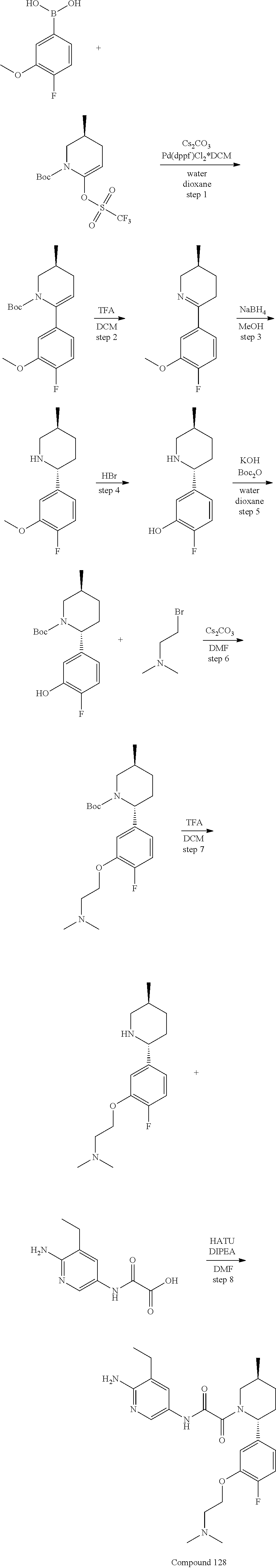

### Step 1: Synthesis of (S)-tert-butyl 6-(4-fluoro-3-methoxyphenyl)-3-methyl-3,4-dihydropyridine-1(2H)-carboxylate

Prepared by general procedure scheme 7.1 step 2. Yield: 10.6 g of crude.

LCMS(ESI): [M-Boc]⁺ m/z: calcd 221.2; found 222.2; Rt=1.567 min.

### Step 2: Synthesis of (S)-6-(4-fluoro-3-methoxyphenyl)-3-methyl-2,3,4,5-tetrahydropyridine

Prepared by general procedure scheme 7.1 step 3. Yield: 5.6 g of crude.

LCMS(ESI): [M]⁺ m/z: calcd 221.2; found 222.2; Rt=0.806 min.

### Step 3: Synthesis of (2R,5S)-2-(4-fluoro-3-methoxyphenyl)-5

Prepared by general procedure scheme 7.1 step 4. Yield: 4.5 g of crude.

LCMS(ESI): [M]⁺ m/z: calcd 223.2; found 224.2; Rt=0.850 min.

### Step 4: Synthesis of 2-fluoro-5-((2R,5S)-5-methylpiperidin-2-yl)phenol

To (2R,5S)-2-(4-fluoro-3-methoxy-phenyl)-5-methyl-piperidine (4.50 g, 20.15 mmol) was added hydrogen bromide (33.97 g, 201.54 mmol, 48% purity) (water solution) and heated for 24 hr. Upon completion, the reaction mixture was concentrated under reduced pressure to obtain crude product as brown color state. The desired product 2-fluoro-5-[(2R,5S)-5-methyl-2-piperidyl]phenol (5.5 g, 18.95 mmol, 94.05% yield, HBr) was isolated as a brown color state.

LCMS(ESI): [M]⁺ m/z: calcd 209.2; found 210.2; Rt=0.657 min.

### Step 5: Synthesis of (2R,5S)-tert-butyl 2-(4-fluoro-3-hydroxyphenyl)-5-methylpiperidine-1-carboxylate

To a stirred solution of 2-fluoro-5-[(2R,5S)-5-methyl-2-piperidyl]phenol (3.3 g, 11.37 mmol, HBr) in dioxane (10.71 mL)/water (10.71 mL) were added potassium hydroxide (1.91 g, 34.12 mmol, 936.50 μL), then reaction mixture was cooled to 0° C. and tert-butoxycarbonyl tert-butyl carbonate (2.61 g, 11.94 mmol, 2.74 mL) was added dropwise. Then resulting reaction mixture was stirred at 25° C. for 12 hr. Upon completion, the reaction mixture was evaporated, then extracted with DCM/H₂O (100 ml/100 ml), organic layer was washed with water (40 ml) and dried over Na₂SO₄ and evaporated. The desired product tert-butyl (2R,5S)-2-(4-fluoro-3-hydroxy-phenyl)-5-methyl-piperidine-1-carboxylate (2.3 g, 7.43 mmol, 65.37% yield) was isolated.

LCMS(ESI): [M]⁺ m/z: calcd 309.2; found 310.2; Rt=1.510 min.

### Step 6: Synthesis of (2R,5S)-tert-butyl 2-(3-(2-(dimethylamino)ethoxy)-4-fluorophenyl)-5-methylpiperidine-1-carboxylate

To a stirred solution of tert-butyl (2R,5S)-2-(4-fluoro-3-hydroxy-phenyl)-5-methyl-piperidine-1-carboxylate (2.3 g, 7.43 mmol) in DMF (25 mL) were added 2-bromo-N,N-dimethyl-ethanamine (8.66 g, 37.17 mmol, HBr) and cesium carbonate (24.22 g, 74.34 mmol) respectively at 80° C. The resulting reaction mixture was stirred at 80° C. for 16 hr. Upon completion, the reaction mixture was quenched with water 150 mL. The aqueous phase was extracted with MTBE 100 mL. The combined organic phase was washed with water 50 mL, brine 50 mL, dried over Na₂SO₄ and concentrated under reduced pressure to obtain crude product as brown color state. The desired product tert-butyl (2R,5S)-2-[3-[2-(dimethylamino)ethoxy]-4-fluoro-phenyl]-5-methyl-piperidine-1-carboxylate (1.3 g, 3.42 mmol, 45.96% yield) was isolated as a brown color state.

LCMS(ESI): [M]⁺ m/z: calcd 380.2; found 381.2; Rt=1.277 min.

### Step 7: Synthesis of 2-(2-fluoro-5-((2R,5S)-5-methylpiperidin-2-yl)phenoxy)-N,N-dimethylethanamine

Prepared by general procedure scheme 7.1 step 3. Yield: 29 mg (3.03%).

HPLC conditions: Column: YMC Triart C18 100*20 mm, 5 microM; 0-1-6 min 50-50-65% water-MeOH+0.1% NH₄OH; (loading pump 4 ml/min MeOH).

LCMS(ESI): [M]⁺ m/z: calcd 280.2; found 281.2; Rt=1.331 min.

### Step 8: Synthesis of N-(6-amino-5-ethylpyridin-3-yl)-2-((2R,5S)-2-(3-(2-(dimethylamino)ethoxy)-4-fluorophenyl)-5-methylpiperidin-1-yl)-2-oxoacetamide (Compound 128)

Prepared by general procedure scheme 7.1 step 5B. Yield: 14.9 mg (27.83%).

HPLC conditions: Column: Chromatorex C18 100*19 mm, 5 microM; 0-5 min 0-30% water-MeCN+0.1% FA 30 ml/min; (loading pump 4 ml/min MeCN).

Compound 128: ¹H NMR (600 MHz, DMSO-d₆) δ (ppm) 1.00 (m, 3H), 1.10 (m, 3H), 1.30 (m, 1H), 1.68 (m, 1H), 1.88 (m, 1H), 2.05 (m, 1H), 2.19 (m, 6H), 2.38 (m, 3H), 2.62 (m, 2H), 3.73 (m, 2H), 4.10 (m, 2H), 5.31 (m, 1H), 5.62 (m, 2H), 6.87 (m, 1H), 7.04 (m, 1H), 7.19 (m, 1H), 7.46 (m, 1H), 8.02 (m, 1H), 10.52 (m, 1H).

LCMS(ESI): [M]⁺ m/z: calcd 471.2; found 472.2; Rt=2.060 min.

### Example 96. Compound 129 N-(6-amino-5-ethyl-3-pyridyl)-2-oxo-2-[(2R,5S)-5-methyl-2-[2-[1-(trideuterio(1¹³C)methyl)-4-piperidyl]-1,3-benzothiazol-5-yl]-1-piperidyl]acetamide

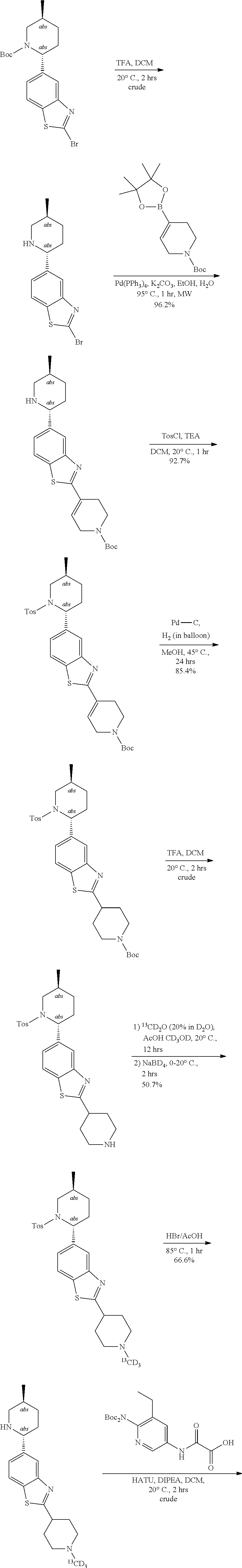

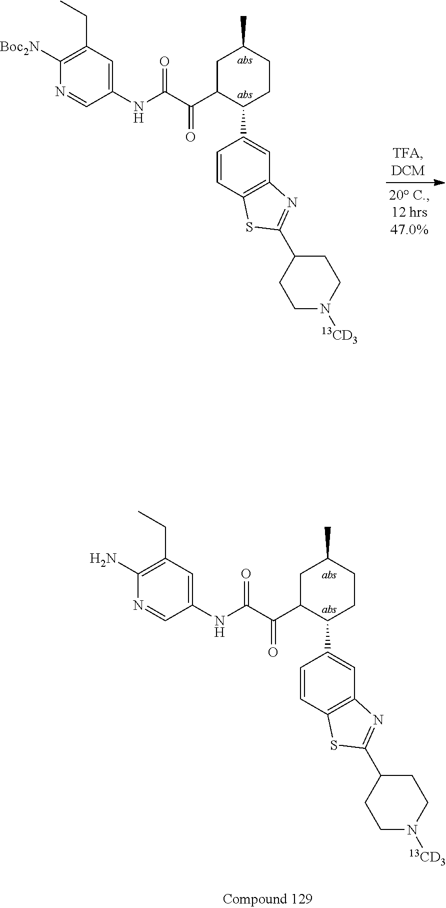

### Step 1: Synthesis of 2-bromo-5-[(2R,5S)-5-methyl-2-piperidyl]-1,3-benzothiazole

A mixture of tert-butyl (2R,5S)-2-(2-bromo-1,3-benzothiazol-5-yl)-5-methyl-piperidine-1-carboxylate (2.3 g, 5.59 mmol), DCM (20 mL) and TFA (4.3 mL, 55.9 mmol) was stirred at 20° C. for 2 hours. The resulting mixture was quenched by addition of water (50 mL), then adjusted to pH=9 with saturated K₂CO₃ aqueous solution. The mixture was extracted with DCM (100 mL*3). The combined organic layer was washed with brine (100 mL), dried over anhydrous Na₂SO₄, filtered and concentrated under reduced pressure to give 2-bromo-5-[(2R,5S)-5-methyl-2-piperidyl]-1,3-benzothiazole (1.7 g, crude) as yellow solid.

### Step 2: Synthesis of tert-butyl 4-[5-[(2R,5S)-5-methyl-2-piperidyl]-1,3-benzothiazol-2-yl]-3,6-dihydro-2H-pyridine-1-carboxylate

To a mixture of 2-bromo-5-[(2R,5S)-5-methyl-2-piperidyl]-1,3-benzothiazole (1.8 g, 5.78 mmol) and tert-butyl 4-(4,4,5,5-tetramethyl-1,3,2-dioxaborolan-2-yl)-3,6-dihydro-2H-pyridine-1-carboxylate (2.15 g, 6.94 mmol) in EtOH (12 mL) and H₂O (5 mL) were added Pd(PPh₃)₄ (669 mg, 0.579 mmol) and K₂CO₃ (2.40 g, 17.4 mmol). The resulting mixture was stirred at 95° C. for 1 hour under microwave. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure. The residue was purified by flash chromatography (ISCO®; 40 g AgelaFlash® Silica Flash Column, DCM/MeOH with MeOH from 0˜10%, Flow Rate: 30 mL/min, 254 nm) to afford tert-butyl 4-[5-[(2R,5S)-5-methyl-2-piperidyl]-1,3-benzothiazol-2-yl]-3,6-dihydro-2H-pyridine-1-carboxylate (2.3 g, 96.2% yield) as yellow oil. LCMS (ESI) [M+H]⁺ m/z: calcd 414.2, found 414.3.

### Step 3: Synthesis of tert-butyl 4-[5-[(2R,5S)-5-methyl-1-(p-tolylsulfonyl)-2-piperidyl]-1,3-benzothiazol-2-yl]-3,6-dihydro-2H-pyridine-1-carboxylate

To a mixture of tert-butyl 4-[5-[(2R,5S)-5-methyl-2-piperidyl]-1,3-benzothiazol-2-yl]-3,6-dihydro-2H-pyridine-1-carboxylate (2.2 g, 5.32 mmol) in DCM (25 mL) were added TEA (2.20 mL, 15.8 mmol) and 4-methylbenzenesulfonyl chloride (1.23 g, 6.46 mmol) slowly. The resulting mixture was stirred at 20° C. for 1 hour. The resulting mixture was quenched by addition of water (50 mL) and extracted with DCM (100 mL*3). The combined organic layer was washed with saturated NH₄Cl aqueous solution (50 mL), brine (100 mL), dried over anhydrous Na₂SO₄, filtered and concentrated under reduced pressure.

The residue was purified by flash chromatography (ISCO®; 40 g AgelaFlash® Silica Flash Column, petroleum ether/EtOAc with EtOAc from 0˜20%, flow rate=30 mL/min, 254 nm) to afford tert-butyl 4-[5-[(2R,5S)-5-methyl-1-(p-tolylsulfonyl)-2-piperidyl]-1,3-benzothiazol-2-yl]-3,6-dihydro-2H-pyridine-1-carboxylate (2.8 g, 92.7% yield) as white solid. ¹H NMR (400 MHz, methanol-d₄) δ ppm 7.71-7.83 (m, 2H), 7.49 (br d, J=8.3 Hz, 2H), 7.32 (br d, J=8.5 Hz, 1H), 7.17-7.27 (m, 2H), 6.76 (br s, 1H), 4.17 (br s, 1H), 3.64-3.72 (m, 2H), 3.06 (br dd, J=12.8, 5.8 Hz, 1H), 2.76 (br s, 1H), 2.30-2.39 (m, 3H), 2.04 (br d, J=5.0 Hz, 2H), 1.86 (br d, J=4.8 Hz, 1H), 1.71 (br d, J=5.0 Hz, 1H), 1.39-1.59 (m, 10H), 1.26 (s, 2H), 1.14-1.22 (m, 2H), 0.89 (br d, J=7.0 Hz, 3H); LCMS (ESI) [M+H]⁺ m/z: calcd 568.2, found 568.4.

### Step 4: Synthesis of tert-butyl 4-[5-[(2R,5S)-5-methyl-1-(p-tolylsulfonyl)-2-piperidyl]-1,3-benzothiazol-2-yl]piperidine-1-carboxylate

A mixture of tert-butyl 4-[5-[(2R,5S)-5-methyl-1-(p-tolylsulfonyl)-2-piperidyl]-1,3-benzothiazol-2-yl]-3,6-dihydro-2H-pyridine-1-carboxylate (2.8 g, 4.93 mmol) and Pd/C (2.80 g, 10 wt % Pd with 50 wt % water) in MeOH (30 mL) was stirred at 45° C. for 24 hours under hydrogen (in balloon). The resulting mixture was filtered and concentrated under reduced pressure to give tert-butyl 4-[5-[(2R,5S)-5-methyl-1-(p-tolylsulfonyl)-2-piperidyl]-1,3-benzothiazol-2-yl]piperidine-1-carboxylate (2.4 g, 85.4% yield) as a white solid. LCMS (ESI) [M+H]⁺ m/z: calcd 570.2, found 470.3. Boc cleaved mass.

### Step 5: Synthesis of 2-(4-piperidyl)-5-[(2R,5S)-5-methyl-1-(p-tolylsulfonyl)-2-piperidyl]-1,3-benzothiazole

A mixture of tert-butyl 4-[5-[(2R,5S)-5-methyl-1-(p-tolylsulfonyl)-2-piperidyl]-1,3-benzothiazol-2-yl]piperidine-1-carboxylate (2.4 g, 4.21 mmol), DCM (20 mL) and TFA (3.26 mL, 42.3 mmol) was stirred at 20° C. for 2 hours. The resulting mixture was adjusted to pH=8 with NH₃—H₂O (12 N). The resulting mixture was quenched by addition of water (100 mL) and extracted with DCM (100 mL*3). The combined organic layer was washed with brine (100 mL), dried over anhydrous Na₂SO₄, filtered and concentrated under reduced pressure to give 2-(4-piperidyl)-5-[(2R,5S)-5-methyl-1-(p-tolylsulfonyl)-2-piperidyl]-1,3-benzothiazole (2 g, crude) as yellow solid, which was used to the next step without further purification.

### Step 6: Synthesis of 5-[(2R,5S)-5-methyl-1-(p-tolylsulfonyl)-2-piperidyl]-2-[1-(trideuterio(1¹³C)methyl)-4-piperidyl]-1,3-benzothiazole

To a mixture of 2-(4-piperidyl)-5-[(2R,5S)-5-methyl-1-(p-tolylsulfonyl)-2-piperidyl]-1,3-benzothiazole (1.9 g, 4.05 mmol) in CD₃OD (20 mL) were added AcOH (0.228 mL, 4.05 mmol) and ¹³CD₂O(20% in D₂O) (3.55 g, 21.5 mmol, 20% purity). The resulting mixture was stirred at 20° C. for 12 hours. NaBD₄ (500 mg, 11.95 mmol) was added at 0° C. The mixture was stirred at 20° C. for 2 hours. The resulting mixture was quenched by addition of water (50 mL) and extracted with EtOAc (100 mL*3). The combined organic layer was washed with brine (50 mL), dried over anhydrous Na₂SO₄, filtered and concentrated under reduced pressure. The residue was purified by flash chromatography (ISCO®; 12 g AgelaFlash® Silica Flash Column, DCM/MeOH with MeOH from 0˜13%, flow rate=30 mL/min, 254 nm) to afford 5-[(2R,5S)-5-methyl-1-(p-tolylsulfonyl)-2-piperidyl]-2-[1-(trideuterio(1¹³C)methyl)-4-piperidyl]-1,3-benzothiazole (1 g, 50.7% yield) as white solid ¹H NMR (400 MHz, methanol-d₄) δ ppm 7.77-7.86 (m, 2H), 7.54 (d, J=8.3 Hz, 2H), 7.35 (dd, J=8.4, 1.6 Hz, 1H), 7.25 (d, J=8.0 Hz, 2H), 4.79 (t, J=5.4 Hz, 1H), 3.63 (dd, J=12.8, 3.8 Hz, 1H), 3.29 (br s, 1H), 3.06 (dd, J=12.8, 5.8 Hz, 1H), 2.74 (brt, J=11.9 Hz, 2H), 2.38 (s, 3H), 2.27-2.35 (m, 2H), 1.98-2.18 (m, 4H), 1.94 (s, 2H), 1.85 (br dd, J=10.8, 4.5 Hz, 1H), 1.68 (td, J=9.0, 4.4 Hz, 1H), 1.18 (td, J=6.7, 4.1 Hz, 1H), 0.86 (d, J=6.8 Hz, 3H); LCMS (ESI) [M+H]⁺ m/z: calcd 488.2, found 488.3; 3D Ratio: 96.58%.

### Step 7: Synthesis of 5-[(2R,5S)-5-methyl-2-piperidyl]-2-[1-(trideuterio(1¹³C)methyl)-4-piperidyl]-1,3-benzothiazole

A mixture of 5-[(2R,5S)-5-methyl-1-(p-tolylsulfonyl)-2-piperidyl]-2-[1-(trideuterio(1¹³C)methyl)-4-piperidyl]-1,3-benzothiazole (900 mg, 1.84 mmol) in HBr/AcOH (1.23 mmol, 4 mL) was stirred at 85° C. for 1 hour. The resulting mixture was concentrated under reduced pressure to removed HBr/AcOH and adjusted to pH=9 with saturated Na₂CO₃ aqueous solution. The solution was stirred at 25° C. for 12 hours. The resulting mixture was quenched by addition of water (100 mL) and extracted with EtOAc (DCM mL*3). The combined organic layer was dried over anhydrous Na₂SO₄, filtered and concentrated under reduced pressure to give residue which was purified by flash chromatography (Column: SepaFlash® Sphercial Cis, 40 g, 40-60 μm, 120A; MeCN/water (0.05 v % NH₃—H₂O) with MeCN from 0-60%, 35 mL/min, 254 nm). The fraction was concentrated under reduced pressure and then lyophilized for overnight to afford 5-[(2R,5S)-5-methyl-2-piperidyl]-2-[1-(trideuterio(1¹³C)methyl)-4-piperidyl]-1,3-benzothiazole (410 mg, 66.6% yield) as white solid. LCMS (ESI) [M+H]⁺ m/z: calcd 334.2, found 334.2; 3D ratio:95.96%.

### Step 8: Synthesis of tert-butyl N-tert-butoxycarbonyl-N-[3-ethyl-5-[[2-oxo-2-[(2R,5S)-5-methyl-2-[2-[1-(trideuterio(1³C)methyl)-4-piperidyl]-1,3-benzothiazol-5-yl]-1-piperidyl]acetyl]amino]-2-pyridyl]carbamate

A mixture of 5-[(2R,5S)-5-methyl-2-piperidyl]-2-[1-(trideuterio(1¹³C)methyl)-4-piperidyl]-1,3-benzothiazole (300 mg, 0.900 mmol), 2-[[6-[bis(tert-butoxycarbonyl)amino]-5-ethyl-3-pyridyl]amino]-2-oxo-acetic acid (370 mg, 0.904 mmol) in DCM (8 mL) were added HATU (410 mg, 1.08 mmol) and DIPEA (0.470 mL, 2.70 mmol). The mixture was stirred at 20° C. for 2 hours. The resulting mixture was quenched by addition of water (50 mL) and extracted with DCM (50 mL*3). The combined organic layer was washed with brine (100 mL), dried over anhydrous Na₂SO₄, filtered and concentrated under reduced pressure. The residue was purified by flash chromatography (ISCO®; 20 g AgelaFlash® Silica Flash Column, DCM/MeOH with MeOH from 0˜10%, flow rate=30 mL/min, 254 nm) to afford tert-butyl N-tert-butoxycarbonyl-N-[3-ethyl-5-[[2-oxo-2-[(2R,5S)-5-methyl-2-[2-[1-(trideuterio(1¹³C)methyl)-4-piperidyl]-1,3-benzothiazol-5-yl]-1-piperidyl]acetyl]amino]-2-pyridyl]carbamate (700 mg, crude) as yellow solid.

### Step 9: Synthesis of N-(6-amino-5-ethyl-3-pyridyl)-2-oxo-2-[(2R,5S)-5-methyl-2-[2-[1-(trideuterio(1¹³C)methyl)-4-piperidyl]-1,3-benzothiazol-5-yl]-1-piperidyl]acetamide (Compound 129)

A mixture tert-butyl N-tert-butoxycarbonyl-N-[3-ethyl-5-[[2-oxo-2-[(2R,5S)-5-methyl-2-[2-[1-(trideuterio(1¹³C)methyl)-4-piperidyl]-1,3-benzothiazol-5-yl]-1-piperidyl]acetyl]amino]-2-pyridyl]carbamate (700 mg, 0.966 mmol), TFA (2.00 mL, 26.0 mmol) and DCM (5 mL) was stirred at 20° C. for 12 hours. The mixture was adjusted to pH=9 with NH₃—H₂O (12N). The mixture was concentrated under reduced pressure and purified by (Column: SepaFlash® Sphercial C₁₈, 25 g, 40-60 m, 120A; MeCN/water (0.5% NH₃—H₂O) with MeCN from 0-45%, 25 mL/min, 220 nm) to give N-(6-amino-5-ethyl-3-pyridyl)-2-oxo-2-[(2R,5S)-5-methyl-2-[2-[1-(trideuterio(1¹³C)methyl)-4-piperidyl]-1,3-benzothiazol-5-yl]-1-piperidyl]acetamide (182.2 mg, 36.0% yield) and N-(6-amino-5-ethyl-3-pyridyl)-2-oxo-2-[(2R,5S)-5-methyl-2-[2-[1-(trideuterio(1¹³C)methyl)-4-piperidyl]-1,3-benzothiazol-5-yl]-1-piperidyl]acetamide (56 mg, 11.0% yield) as white solid.

First crop: ¹H NMR (400 MHz, methanol-d₄) δ ppm 7.89-8.17 (m, 3H), 7.39-7.69 (m, 2H), 5.47-5.86 (m, 1H), 3.75 (br d, J=13.1 Hz, 1H), 3.43 (br d, J=12.8 Hz, 1H), 3.21 (br d, J=11.8 Hz, 3H), 2.41-2.64 (m, 4H), 2.28 (br d, J=11.8 Hz, 4H), 1.91-2.10 (m, 4H), 1.47 (br d, J=10.0 Hz, 1H), 1.17-1.30 (m, 3H), 1.14 (br d, J=6.8 Hz, 3H); LCMS (ESI) [M+H]⁺ m/z: calcd 525.3, found 525.4; HPLC: 100%@254 nm; 100% ee; 3D Ratio: 95.8%.

Second crop: ¹H NMR (400 MHz, methanol-d₄) δ ppm 7.86-8.20 (m, 3H), 7.40-7.71 (m, 2H), 5.45-5.85 (m, 1H), 3.76 (br d, J=12.8 Hz, 1H), 3.43 (br d, J=11.4 Hz, 1H), 3.17 (br s, 1H), 3.03 (br d, J=10.5 Hz, 2H), 2.41-2.61 (m, 2H), 2.20-2.38 (m, 6H), 1.98 (br d, J=11.4 Hz, 4H), 1.47 (br d, J=12.3 Hz, 1H), 1.11-1.31 (m, 6H); LCMS (ESI) [M+H]⁺ m/z: calcd 525.3, found 525.4; HPLC: 100%@254 nm; 100% ee; 3D Ratio: 95.77%.

### Example 97. Cellular Assay—SDMA In-Cell Western Protocol

A HAP1 MTAP-isogenic cell line pair was acquired from Horizon Discovery (HZGHC004894c005) and maintained in DMEM (ThermoFisher 11965)+10% FBS (Gemini 100-500) in a humidified, 10% CO2 tissue culture incubator. The SAM-cooperative PRMT5 inhibitor, GSK3326595, was sourced from SelleckChem and maintained as a 10 mM DMSO stock. All test compounds are maintained as 10 mM DMSO stocks

On Day 0, MTAP-intact or MTAP-deleted cells are seeded in a 384-well plate, and incubated in a humidified, 5% CO2 tissue culture incubator for 16-24 hours. On Day 1, the test compounds are dispensed to wells at defined concentrations using a Tecan D300e digital dispenser (n=4), and the volume of DMSO is normalized to highest class volume. Each plate includes wells dosed with defined concentrations of GSK33226595 as a plate control. The compounds are incubated with cells for 24 hours in a humidified, 5% CO2 tissue culture incubator.

On Day 2, the compound-treated cells are fixed with a final concentration of 4% formaldehyde. The cells are then washed/permeabilized with 1×PBS+0.1% Triton X-100, and then blocked with 5% goat serum/1×TBS. The fixed cells are then incubated overnight at 4° C. with a primary SDMA antibody cocktail (Cell Signaling 13222).

On Day 3, the cells are washed with 1×PBS+0.1% Triton X-100, and then incubated at room temperature for 1 hour with a NIR fluorescent secondary antibody cocktail that also contains DRAQ5 (LiCor 926-32211 and VWR 10761-508). The cells are washed with 1×PBS+0.1% Triton X-100, and then washed again with ddH20. The plates are then imaged using a NIR fluorescent imager (LiCor Odyssey).

For data analysis, the SDMA signal is normalized to the DRAQ5 signal. Assay background is determined by the signal from wells treated with 1 μM GSK3326595, and subtracted from every well. The data are plotted as % of the DMSO control wells for the MTAP-intact and the MTAP-deleted cell lines independently, and fitted to the 4-parameter logistic (4-PL) Hill equation with maximal effect constrained to 0. The fit was performed using GraphPad Prism or the default IC50 fitting procedure in Dotmatics Studies 5.4 as part of a customized data analysis protocol.

The data obtained in this experiment is presented in Table 1, columns 4-6.

### Example 98. Viability Assay Protocol

A HAP1 MTAP-isogenic cell line pair was acquired from Horizon Discovery (HZGHC004894c005) and maintained in DMEM (ThermoFisher 11965)+10% FBS (Gemini 100-500) in a humidified, 5 or 10% CO2 tissue culture incubator. All test compounds are maintained as 10 mM DMSO stocks.

On Day 0, MTAP-intact and MTAP-deleted cells are seeded in a 96-well plate, and incubated in a humidified, 5 or 10% CO2 tissue culture incubator for 16-24 hours. On Day 1, the test compounds are dispensed to wells at defined concentrations using a Tecan D300e digital dispenser (n=3), and the volume of DMSO is normalized to highest class volume (0.2%). The compound-treated plates are incubated for 7 days in a humidified, 5 or 10% CO2 tissue culture incubator.

On Day 7, the plates are removed from the tissue culture incubator and allowed to equilibrate to room temperature. Then either a ½% volume CellTiter-Glo Luminescent Cell Viability Assay reagent (Promega G7572) is added to each well, or the media is removed from every well and a 1:3 dilution of CellTiter-Glo 2.0 Cell Viability Assay reagent (Promega G9241) in 1×PBS is added. Ten minutes after addition, the luminescent signal is detected by an Envision plate reader. The data are plotted as % of the DMSO control wells for the MTAP-intact and the MTAP-deleted cell lines independently, and fitted to the 4-parameter logistic (4-PL) Hill equation with maximal effect constrained to 0. The fit was performed using GraphPad Prism or the default IC50 fitting procedure in Dotmatics Studies 5.4 as part of a customized data analysis protocol.

The data obtained in this experiment is presented in Table 1, column 8.

### Example 99. Combination Viability Assay Protocol

A SW1573 MTAP-isogenic cell line pair can be generated by either reconstituting MTAP gene expression, or by introducing an empty control vector, in the MTAP-deleted SW1573 parental cell line. The cell lines can be maintained in DMEM+10% FBS in a humidified, 5% CO2 tissue culture incubator. All test compounds can be maintained as 10 mM DMSO stocks.

On Day 0, MTAP-intact and MTAP-deleted cells can be seeded in a 384-well plate, and incubated in a humidified, 5% CO2 tissue culture incubator for 16-24 hours. On Day 1, the test compounds can be dispensed to wells at defined concentrations (n=2), and the volume of DMSO can be normalized to highest class volume. The compound-treated plates can be incubated for 7 days in a humidified, 5% CO2 tissue culture incubator.

On Day 7, the plates can be removed from the tissue culture incubator and allowed to equilibrate to room temperature. Relative viability can be assessed by addition of CellTiter-Glo reagent, and data can be plotted as % of DMSO control for each compound in each cell line, with a 4-parameter fit non-linear regression model (GraphPad Prism). Synergy can be determined according to the HSA model by the Combenefit software package (Version 2.021).

### PRMT5 Inhibitors and MAT2A Inhibitors Represents a Potential Clinical Combination in MTAP-Deleted Tumors

Marjon et al (Cell Reports 2016) and Kalev et al (Cancer Cell 2021) identify MAT2A as a therapeutic target in MTAP-deleted tumors. The combination of a MAT2A inhibitor with an inhibitor that selectively targets PRMT5 in MTAP-null cells can be assessed to determine whether this would present a rational therapeutic strategy. Combination of a MAT2A inhibitor (e.g., AG-270) with an exemplar MTAP^{null}-selective PRMT5 inhibitor in a 7-day viability assay in the MTAP-null SW1573 cancer cell line can demonstrate enhanced cellular viability defects.

### PRMT5 Inhibitors and MAPK or KRASG12C Inhibitors Represent a Potential Clinical Combination in MTAP-Deleted, KRAS-Mutated Tumors

MTAP-deletion can co-occur with mutations in the KRAS gene (e.g., KRASG12C). Therapies targeting KRAS or other members of the MAPK pathway (eg, MAPK3, MAPK1, MEK1 and MEK2) exist. The combination of these inhibitors with an inhibitor that selectively targets PRMT5 in MTAP-null cells can be assessed to determine whether this would present a therapeutic strategy.

Combination of a KRASG12C inhibitor (e.g., AMG-510), with an exemplar MTAP^{null}-selective PRMT5 inhibitor in a 7-day viability assay in the MTAP-null SW1573 cancer cell line can demonstrate enhanced cellular viability defects.

Combination of MAPK1/MAPK3 inhibitors (e.g., ulixertinib and SCH772984), with an exemplar MTAP^{null}-selective PRMT5 inhibitor in a 7-day viability assay in the MTAP-null SW1573 cancer cell line can demonstrate enhanced cellular viability defects.

Combination of MEK inhibitors (e.g., trametinib) with an exemplar MTAP^{null}-selective PRMT5 inhibitor in a 7-day viability assay in the MTAP-null SW1573 cancer cell line can demonstrate enhanced cellular viability defects.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of the group is present in, employed in, or otherwise relevant to a given product or process. In some embodiments, more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or embodiments of the invention, is/are referred to as comprising particular elements and/or features, certain embodiments of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment that falls within the prior art may be explicitly excluded from any one or more of the claims. Because such embodiments are deemed to be known to one of ordinary skill in the art, they may be excluded even if the exclusion is not set forth explicitly herein. Any particular embodiment be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound selected from the group consisting of 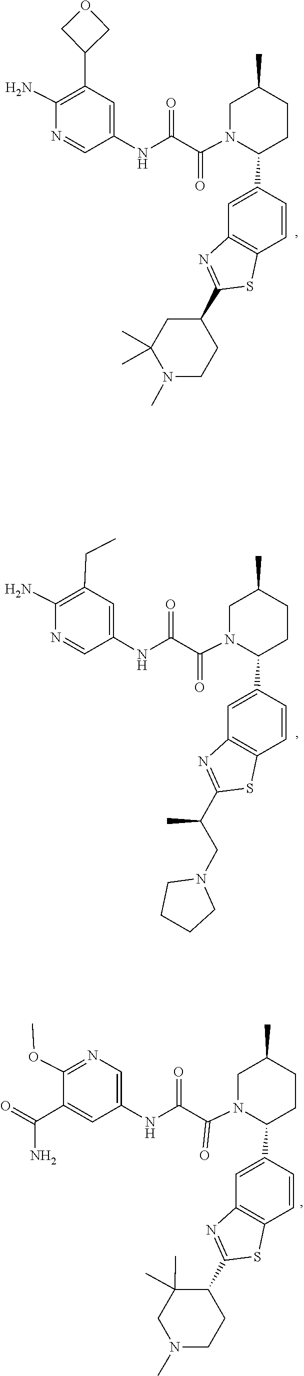 [SMILES: C[C@H]1CC[C@H](c2ccc3sc([C@H]4CCN(C)C(C)(C)C4)nc3c2)N(C(=O)C(=O)Nc2cnc(N)c(C3COC3)c2)C1 ; CCc1cc(NC(=O)C(=O)N2C[C@@H](C)CC[C@@H]2c2ccc3sc([C@H](C)CN4CCCC4)nc3c2)cnc1N ; COc1ncc(NC(=O)C(=O)N2C[C@@H](C)CC[C@@H]2c2ccc3sc([C@@H]4CCN(C)CC4(C)C)nc3c2)cc1C(N)=O] 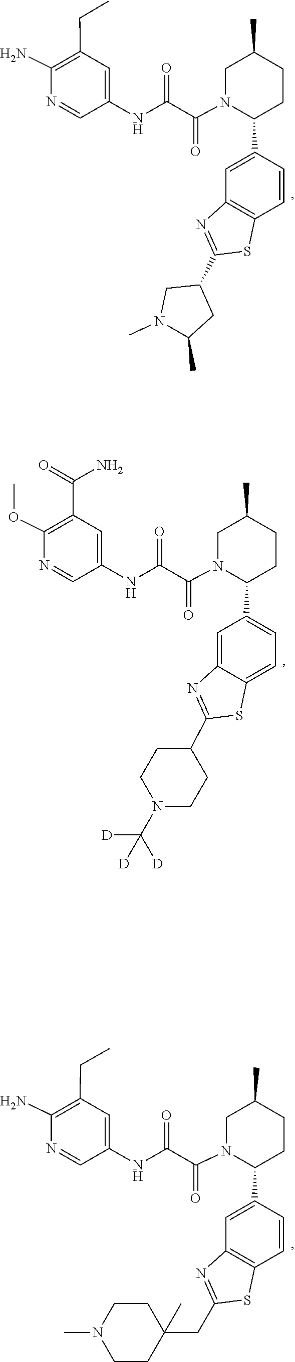 [SMILES: CCc1cc(NC(=O)C(=O)N2C[C@@H](C)CC[C@@H]2c2ccc3sc([C@@H]4C[C@@H](C)N(C)C4)nc3c2)cnc1N ; [2H]C([2H])([2H])N1CCC(c2nc3cc([C@H]4CC[C@H](C)CN4C(=O)C(=O)Nc4cnc(OC)c(C(N)=O)c4)ccc3s2)CC1 ; CCc1cc(NC(=O)C(=O)N2C[C@@H](C)CC[C@@H]2c2ccc3sc(CC4(C)CCN(C)CC4)nc3c2)cnc1N] 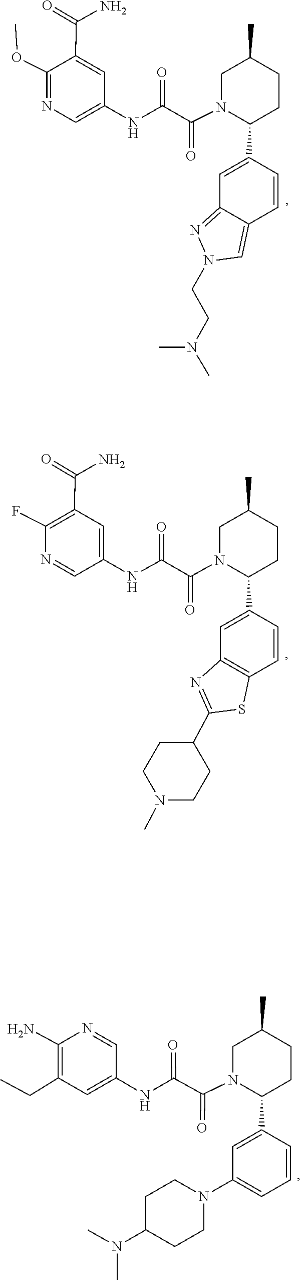 [SMILES: COc1ncc(NC(=O)C(=O)N2C[C@@H](C)CC[C@@H]2c2ccc3cn(CCN(C)C)nc3c2)cc1C(N)=O ; C[C@H]1CC[C@H](c2ccc3sc(C4CCN(C)CC4)nc3c2)N(C(=O)C(=O)Nc2cnc(F)c(C(N)=O)c2)C1 ; CCc1cc(NC(=O)C(=O)N2C[C@@H](C)CC[C@@H]2c2cccc(N3CCC(N(C)C)CC3)c2)cnc1N] 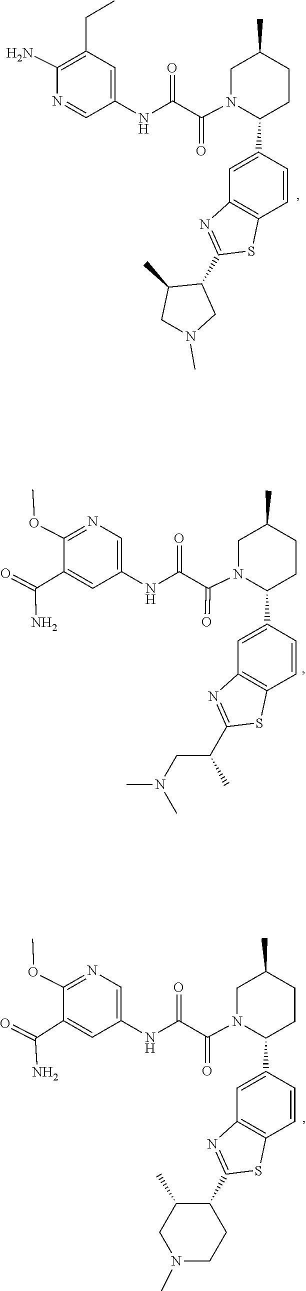 [SMILES: CCc1cc(NC(=O)C(=O)N2C[C@@H](C)CC[C@@H]2c2ccc3sc([C@@H]4CN(C)C[C@H]4C)nc3c2)cnc1N ; COc1ncc(NC(=O)C(=O)N2C[C@@H](C)CC[C@@H]2c2ccc3sc([C@H](C)CN(C)C)nc3c2)cc1C(N)=O ; COc1ncc(NC(=O)C(=O)N2C[C@@H](C)CC[C@@H]2c2ccc3sc([C@@H]4CCN(C)C[C@@H]4C)nc3c2)cc1C(N)=O] 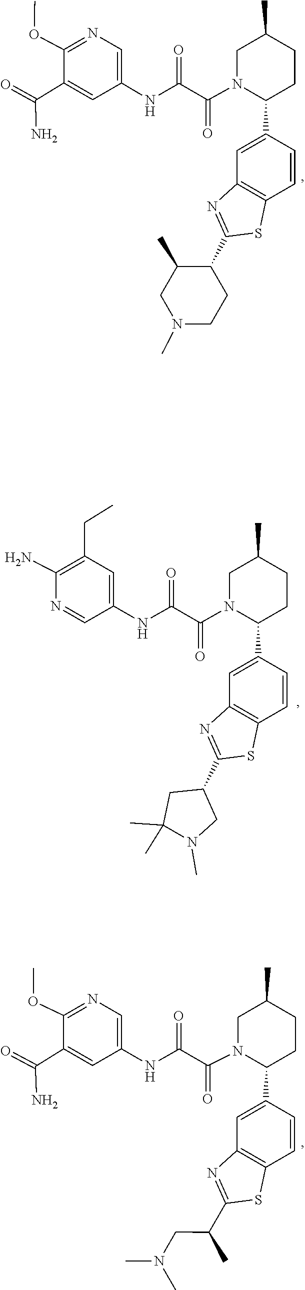 [SMILES: COc1ncc(NC(=O)C(=O)N2C[C@@H](C)CC[C@@H]2c2ccc3sc([C@@H]4CCN(C)C[C@H]4C)nc3c2)cc1C(N)=O ; CCc1cc(NC(=O)C(=O)N2C[C@@H](C)CC[C@@H]2c2ccc3sc([C@@H]4CN(C)C(C)(C)C4)nc3c2)cnc1N ; COc1ncc(NC(=O)C(=O)N2C[C@@H](C)CC[C@@H]2c2ccc3sc([C@@H](C)CN(C)C)nc3c2)cc1C(N)=O] 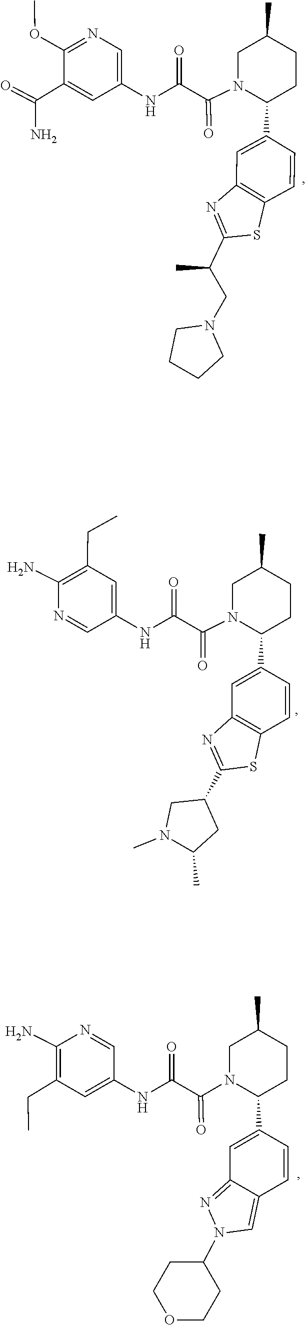 [SMILES: COc1ncc(NC(=O)C(=O)N2C[C@@H](C)CC[C@@H]2c2ccc3sc([C@H](C)CN4CCCC4)nc3c2)cc1C(N)=O ; CCc1cc(NC(=O)C(=O)N2C[C@@H](C)CC[C@@H]2c2ccc3sc([C@@H]4C[C@H](C)N(C)C4)nc3c2)cnc1N ; CCc1cc(NC(=O)C(=O)N2C[C@@H](C)CC[C@@H]2c2ccc3cn(C4CCOCC4)nc3c2)cnc1N] 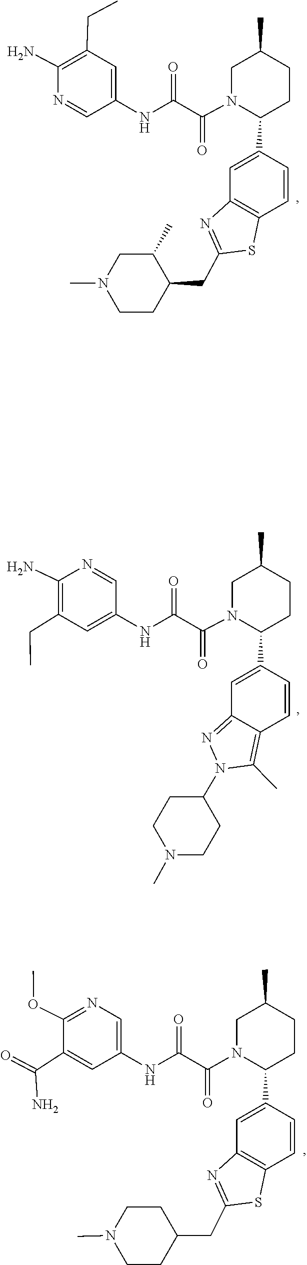 [SMILES: CCc1cc(NC(=O)C(=O)N2C[C@@H](C)CC[C@@H]2c2ccc3sc(C[C@H]4CCN(C)C[C@@H]4C)nc3c2)cnc1N ; CCc1cc(NC(=O)C(=O)N2C[C@@H](C)CC[C@@H]2c2ccc3c(C)n(C4CCN(C)CC4)nc3c2)cnc1N ; COc1ncc(NC(=O)C(=O)N2C[C@@H](C)CC[C@@H]2c2ccc3sc(CC4CCN(C)CC4)nc3c2)cc1C(N)=O] 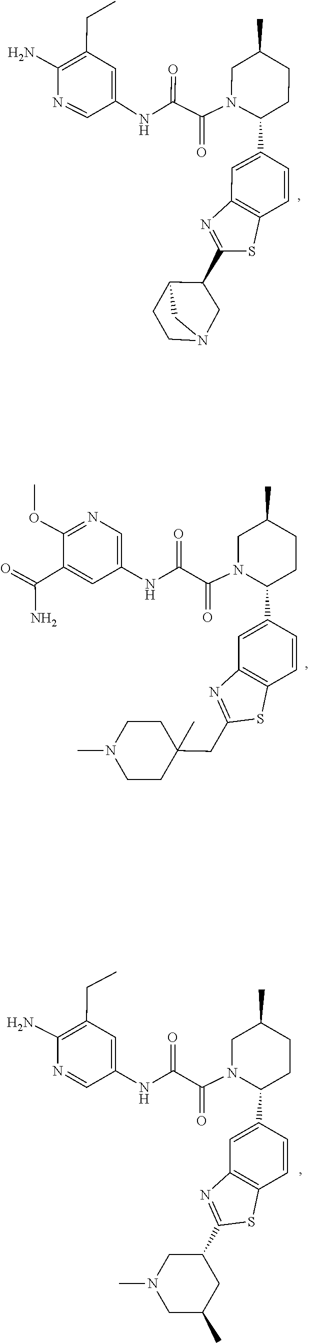 [SMILES: CCc1cc(NC(=O)C(=O)N2C[C@@H](C)CC[C@@H]2c2ccc3sc([C@H]4CN5CC[C@@H]4C5)nc3c2)cnc1N ; COc1ncc(NC(=O)C(=O)N2C[C@@H](C)CC[C@@H]2c2ccc3sc(CC4(C)CCN(C)CC4)nc3c2)cc1C(N)=O ; CCc1cc(NC(=O)C(=O)N2C[C@@H](C)CC[C@@H]2c2ccc3sc([C@@H]4C[C@@H](C)CN(C)C4)nc3c2)cnc1N] 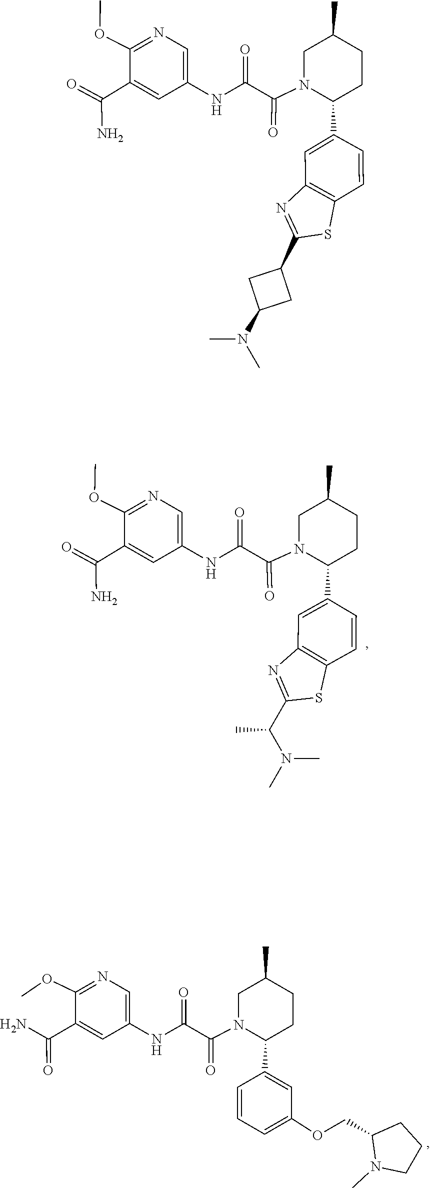 [SMILES: COc1ncc(NC(=O)C(=O)N2C[C@@H](C)CC[C@@H]2c2ccc3sc([C@H]4C[C@@H](N(C)C)C4)nc3c2)cc1C(N)=O ; COc1ncc(NC(=O)C(=O)N2C[C@@H](C)CC[C@@H]2c2ccc3sc([C@@H](C)N(C)C)nc3c2)cc1C(N)=O ; COc1ncc(NC(=O)C(=O)N2C[C@@H](C)CC[C@@H]2c2cccc(OC[C@@H]3CCCN3C)c2)cc1C(N)=O] 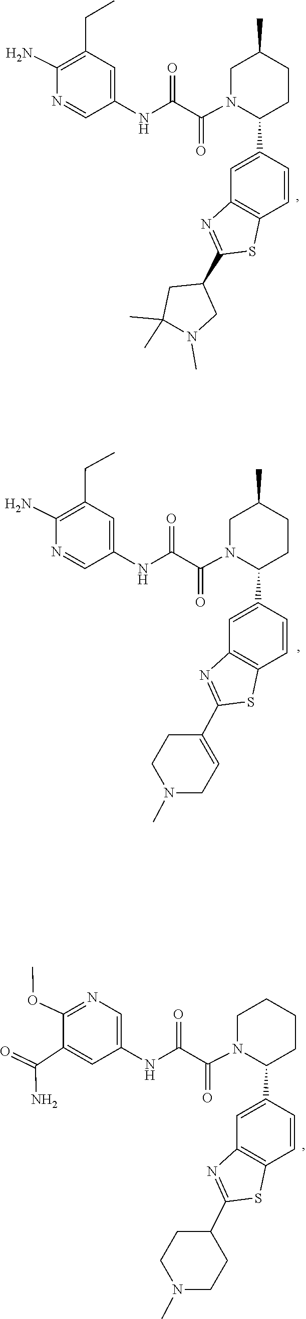 [SMILES: CCc1cc(NC(=O)C(=O)N2C[C@@H](C)CC[C@@H]2c2ccc3sc([C@H]4CN(C)C(C)(C)C4)nc3c2)cnc1N ; CCc1cc(NC(=O)C(=O)N2C[C@@H](C)CC[C@@H]2c2ccc3sc(C4=CCN(C)CC4)nc3c2)cnc1N ; COc1ncc(NC(=O)C(=O)N2CCCC[C@@H]2c2ccc3sc(C4CCN(C)CC4)nc3c2)cc1C(N)=O] 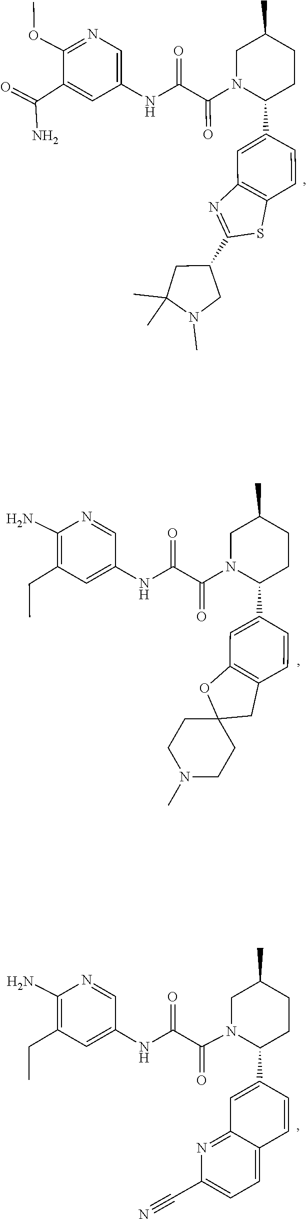 [SMILES: COc1ncc(NC(=O)C(=O)N2C[C@@H](C)CC[C@@H]2c2ccc3sc([C@@H]4CN(C)C(C)(C)C4)nc3c2)cc1C(N)=O ; CCc1cc(NC(=O)C(=O)N2C[C@@H](C)CC[C@@H]2c2ccc3c(c2)OC2(CCN(C)CC2)C3)cnc1N ; CCc1cc(NC(=O)C(=O)N2C[C@@H](C)CC[C@@H]2c2ccc3ccc(C#N)nc3c2)cnc1N] 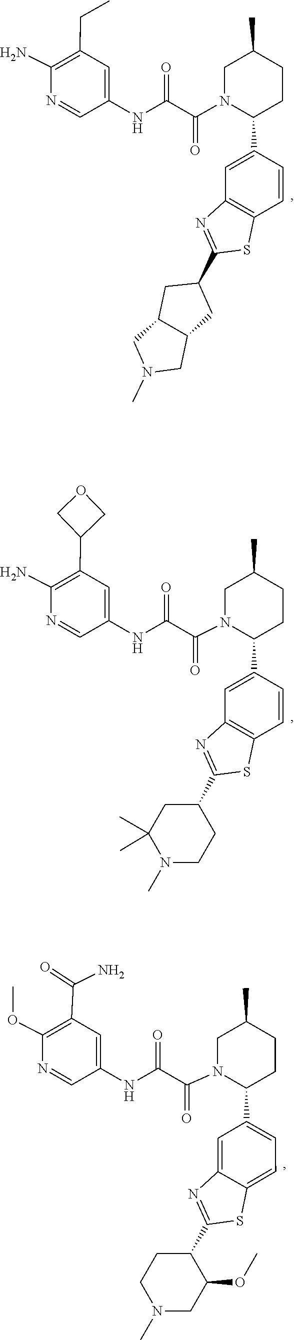 [SMILES: CCc1cc(NC(=O)C(=O)N2C[C@@H](C)CC[C@@H]2c2ccc3sc([C@H]4C[C@@H]5CN(C)C[C@@H]5C4)nc3c2)cnc1N ; C[C@H]1CC[C@H](c2ccc3sc([C@@H]4CCN(C)C(C)(C)C4)nc3c2)N(C(=O)C(=O)Nc2cnc(N)c(C3COC3)c2)C1 ; COc1ncc(NC(=O)C(=O)N2C[C@@H](C)CC[C@@H]2c2ccc3sc([C@H]4CCN(C)C[C@@H]4OC)nc3c2)cc1C(N)=O] 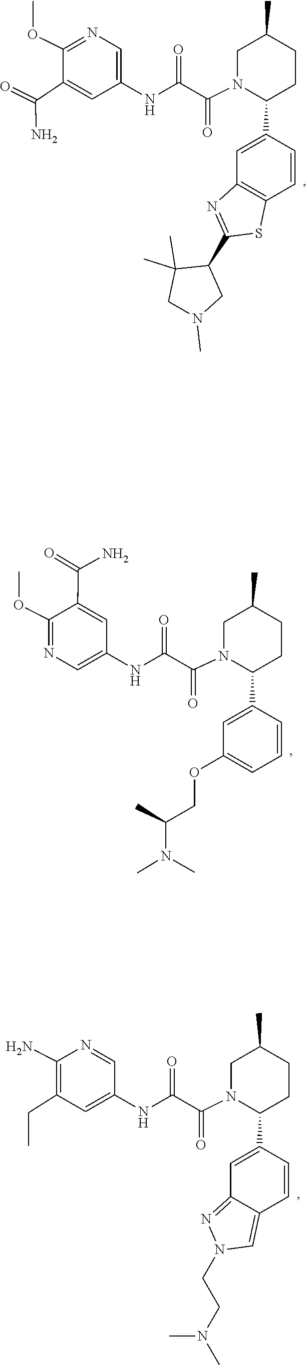 [SMILES: COc1ncc(NC(=O)C(=O)N2C[C@@H](C)CC[C@@H]2c2ccc3sc([C@H]4CN(C)CC4(C)C)nc3c2)cc1C(N)=O ; COc1ncc(NC(=O)C(=O)N2C[C@@H](C)CC[C@@H]2c2cccc(OC[C@H](C)N(C)C)c2)cc1C(N)=O ; CCc1cc(NC(=O)C(=O)N2C[C@@H](C)CC[C@@H]2c2ccc3cn(CCN(C)C)nc3c2)cnc1N] 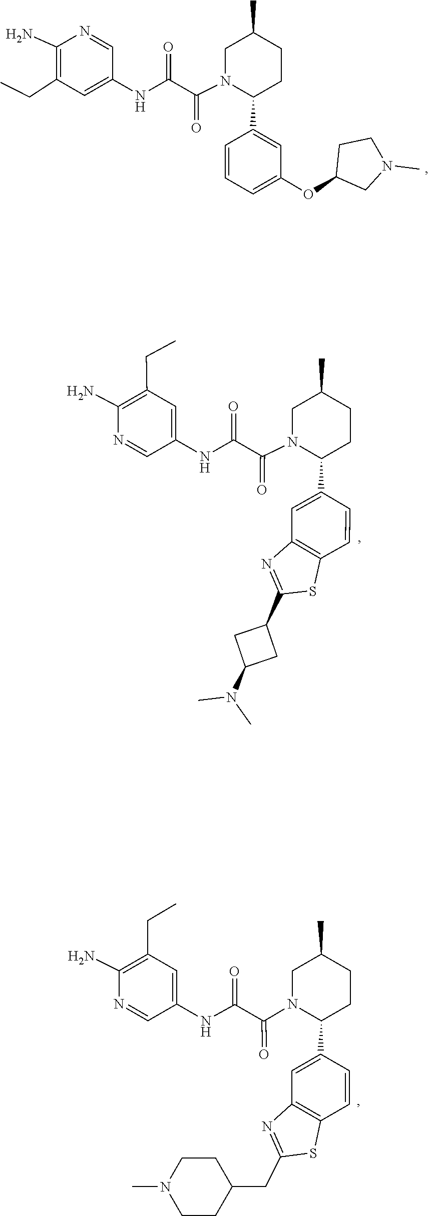 [SMILES: CCc1cc(NC(=O)C(=O)N2C[C@@H](C)CC[C@@H]2c2cccc(O[C@H]3CCN(C)C3)c2)cnc1N ; CCc1cc(NC(=O)C(=O)N2C[C@@H](C)CC[C@@H]2c2ccc3sc([C@H]4C[C@@H](N(C)C)C4)nc3c2)cnc1N ; CCc1cc(NC(=O)C(=O)N2C[C@@H](C)CC[C@@H]2c2ccc3sc(CC4CCN(C)CC4)nc3c2)cnc1N] 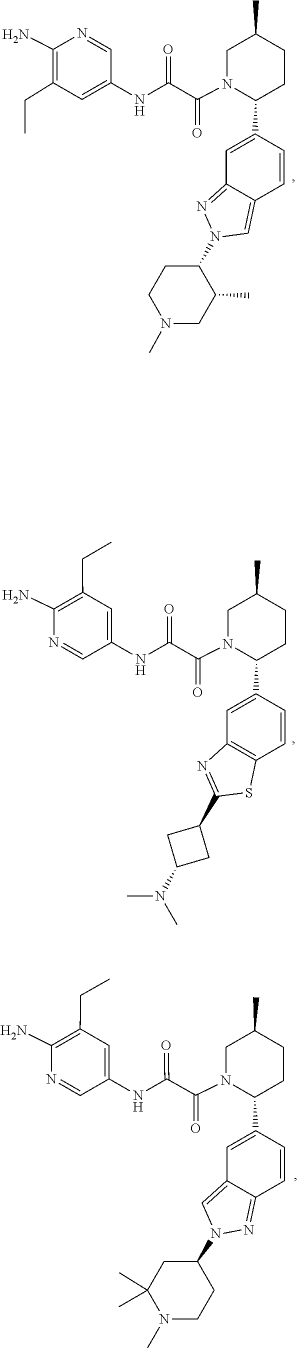 [SMILES: CCc1cc(NC(=O)C(=O)N2C[C@@H](C)CC[C@@H]2c2ccc3cn([C@H]4CCN(C)C[C@H]4C)nc3c2)cnc1N ; CCc1cc(NC(=O)C(=O)N2C[C@@H](C)CC[C@@H]2c2ccc3sc([C@H]4C[C@H](N(C)C)C4)nc3c2)cnc1N ; CCc1cc(NC(=O)C(=O)N2C[C@@H](C)CC[C@@H]2c2ccc3nn([C@H]4CCN(C)C(C)(C)C4)cc3c2)cnc1N] 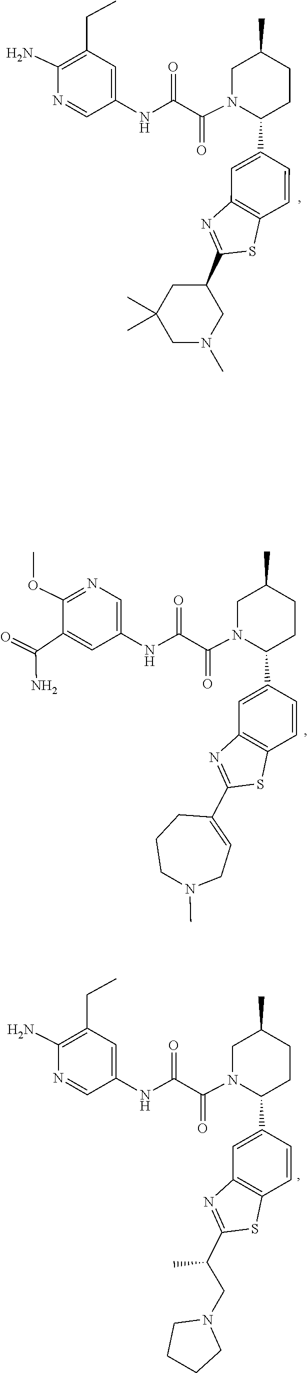 [SMILES: CCc1cc(NC(=O)C(=O)N2C[C@@H](C)CC[C@@H]2c2ccc3sc([C@H]4CN(C)CC(C)(C)C4)nc3c2)cnc1N ; COc1ncc(NC(=O)C(=O)N2C[C@@H](C)CC[C@@H]2c2ccc3sc(C4=CCN(C)CCC4)nc3c2)cc1C(N)=O ; CCc1cc(NC(=O)C(=O)N2C[C@@H](C)CC[C@@H]2c2ccc3sc([C@@H](C)CN4CCCC4)nc3c2)cnc1N] 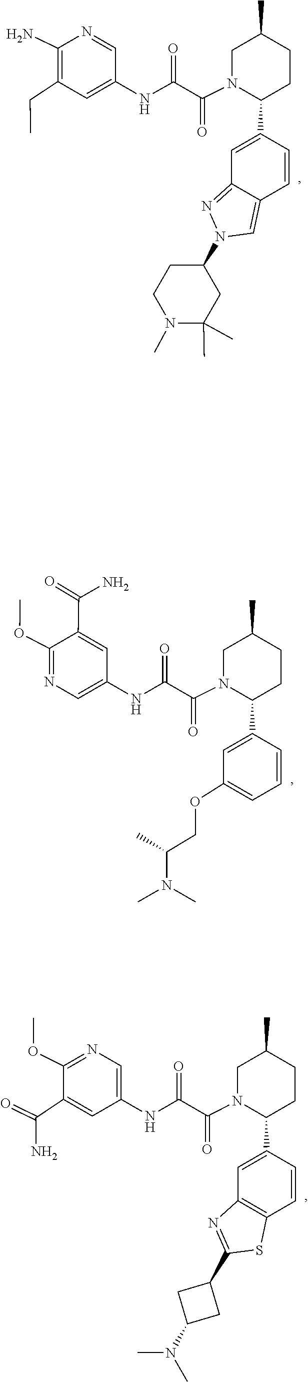 [SMILES: CCc1cc(NC(=O)C(=O)N2C[C@@H](C)CC[C@@H]2c2ccc3cn([C@@H]4CCN(C)C(C)(C)C4)nc3c2)cnc1N ; COc1ncc(NC(=O)C(=O)N2C[C@@H](C)CC[C@@H]2c2cccc(OC[C@@H](C)N(C)C)c2)cc1C(N)=O ; COc1ncc(NC(=O)C(=O)N2C[C@@H](C)CC[C@@H]2c2ccc3sc([C@H]4C[C@H](N(C)C)C4)nc3c2)cc1C(N)=O] 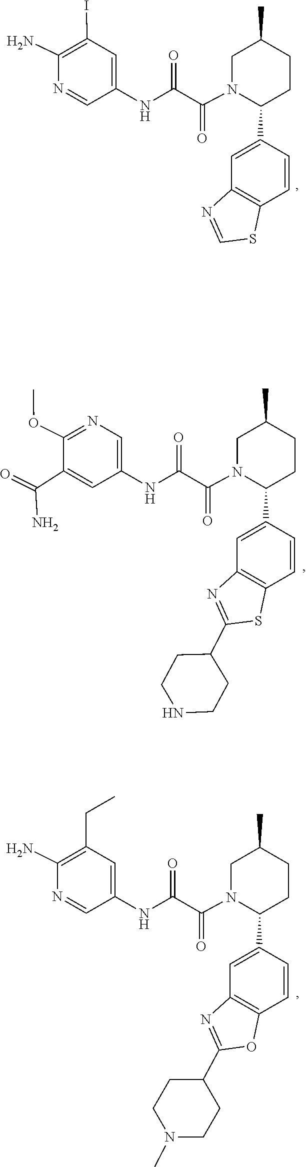 [SMILES: C[C@H]1CC[C@H](c2ccc3scnc3c2)N(C(=O)C(=O)Nc2cnc(N)c(I)c2)C1 ; COc1ncc(NC(=O)C(=O)N2C[C@@H](C)CC[C@@H]2c2ccc3sc(C4CCNCC4)nc3c2)cc1C(N)=O ; CCc1cc(NC(=O)C(=O)N2C[C@@H](C)CC[C@@H]2c2ccc3oc(C4CCN(C)CC4)nc3c2)cnc1N] 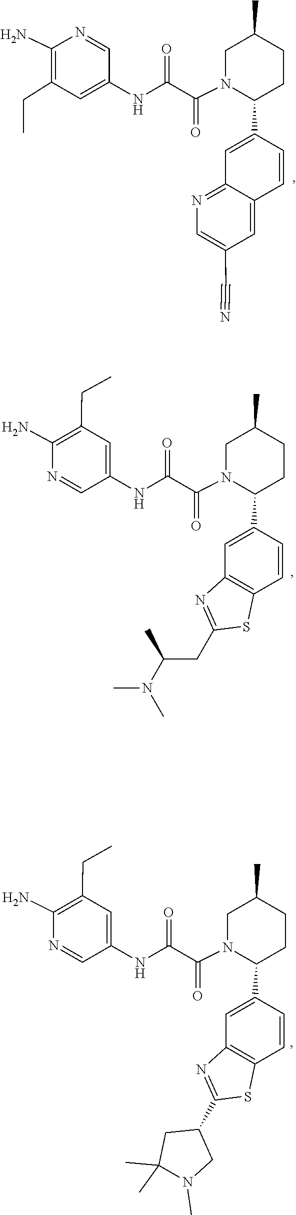 [SMILES: CCc1cc(NC(=O)C(=O)N2C[C@@H](C)CC[C@@H]2c2ccc3cc(C#N)cnc3c2)cnc1N ; CCc1cc(NC(=O)C(=O)N2C[C@@H](C)CC[C@@H]2c2ccc3sc(C[C@H](C)N(C)C)nc3c2)cnc1N ; CCc1cc(NC(=O)C(=O)N2C[C@@H](C)CC[C@@H]2c2ccc3sc([C@@H]4CN(C)C(C)(C)C4)nc3c2)cnc1N] 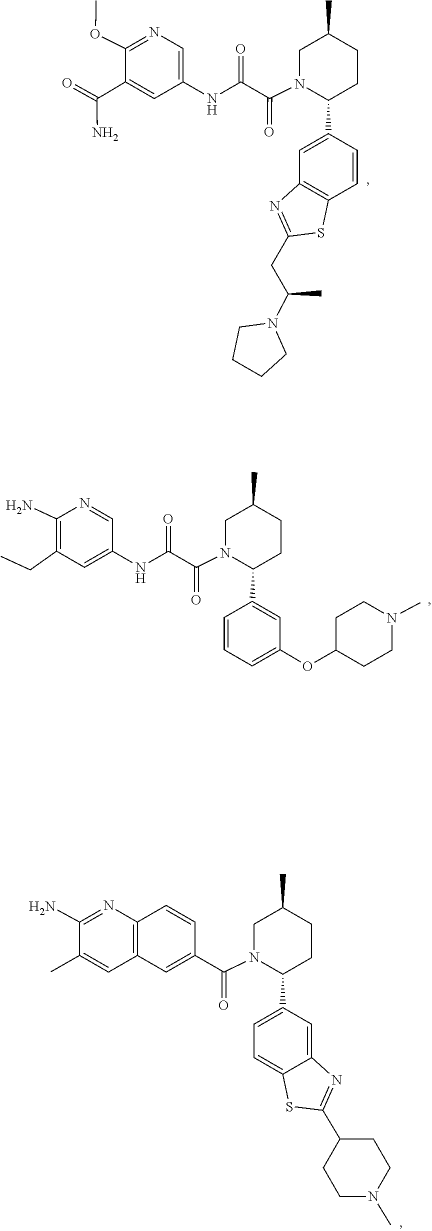 [SMILES: COc1ncc(NC(=O)C(=O)N2C[C@@H](C)CC[C@@H]2c2ccc3sc(C[C@@H](C)N4CCCC4)nc3c2)cc1C(N)=O ; CCc1cc(NC(=O)C(=O)N2C[C@@H](C)CC[C@@H]2c2cccc(OC3CCN(C)CC3)c2)cnc1N ; Cc1cc2cc(C(=O)N3C[C@@H](C)CC[C@@H]3c3ccc4sc(C5CCN(C)CC5)nc4c3)ccc2nc1N] 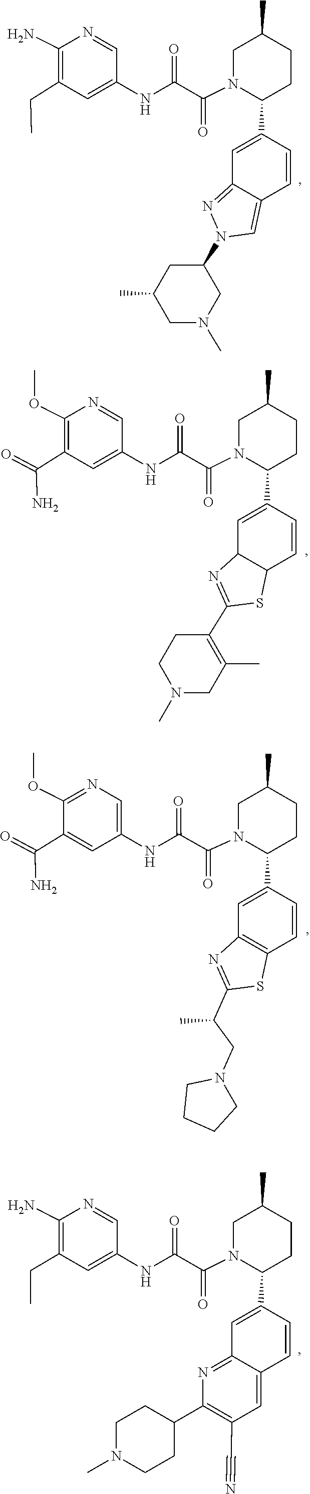 [SMILES: CCc1cc(NC(=O)C(=O)N2C[C@@H](C)CC[C@@H]2c2ccc3cn([C@@H]4C[C@@H](C)CN(C)C4)nc3c2)cnc1N ; COc1ncc(NC(=O)C(=O)N2C[C@@H](C)CC[C@@H]2C2=CC3N=C(C4=C(C)CN(C)CC4)SC3C=C2)cc1C(N)=O ; COc1ncc(NC(=O)C(=O)N2C[C@@H](C)CC[C@@H]2c2ccc3sc([C@@H](C)CN4CCCC4)nc3c2)cc1C(N)=O ; CCc1cc(NC(=O)C(=O)N2C[C@@H](C)CC[C@@H]2c2ccc3cc(C#N)c(C4CCN(C)CC4)nc3c2)cnc1N] 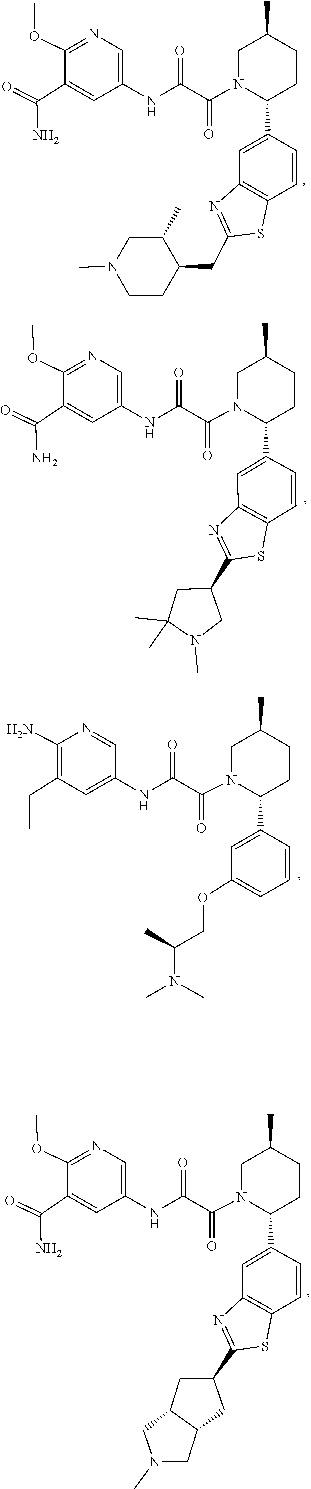 [SMILES: COc1ncc(NC(=O)C(=O)N2C[C@@H](C)CC[C@@H]2c2ccc3sc(C[C@H]4CCN(C)C[C@@H]4C)nc3c2)cc1C(N)=O ; COc1ncc(NC(=O)C(=O)N2C[C@@H](C)CC[C@@H]2c2ccc3sc([C@H]4CN(C)C(C)(C)C4)nc3c2)cc1C(N)=O ; CCc1cc(NC(=O)C(=O)N2C[C@@H](C)CC[C@@H]2c2cccc(OC[C@H](C)N(C)C)c2)cnc1N ; COc1ncc(NC(=O)C(=O)N2C[C@@H](C)CC[C@@H]2c2ccc3sc([C@H]4C[C@@H]5CN(C)C[C@@H]5C4)nc3c2)cc1C(N)=O] 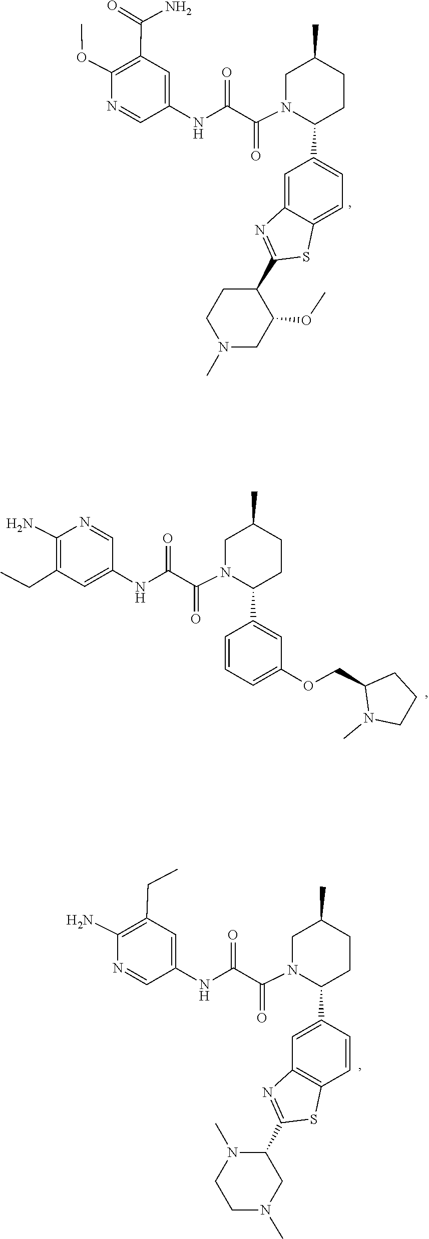 [SMILES: COc1ncc(NC(=O)C(=O)N2C[C@@H](C)CC[C@@H]2c2ccc3sc([C@@H]4CCN(C)C[C@H]4OC)nc3c2)cc1C(N)=O ; CCc1cc(NC(=O)C(=O)N2C[C@@H](C)CC[C@@H]2c2cccc(OC[C@H]3CCCN3C)c2)cnc1N ; CCc1cc(NC(=O)C(=O)N2C[C@@H](C)CC[C@@H]2c2ccc3sc([C@@H]4CN(C)CCN4C)nc3c2)cnc1N] 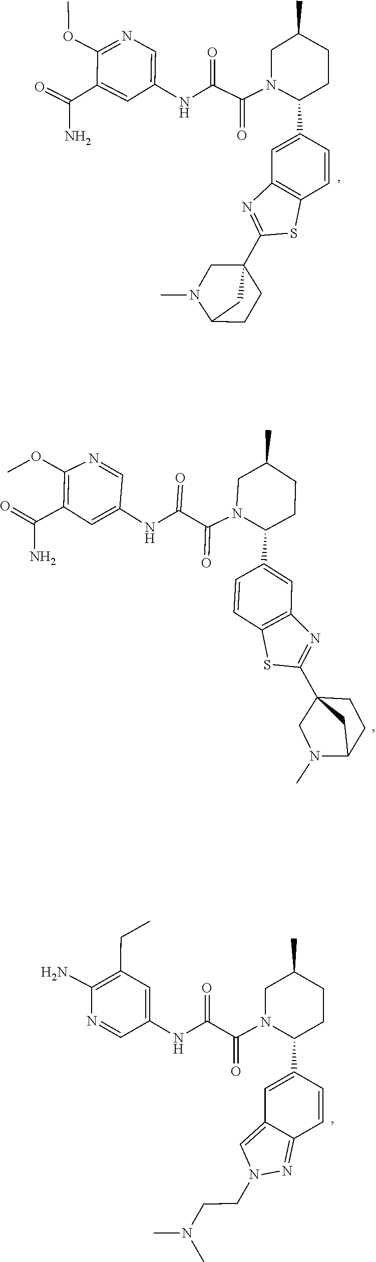 [SMILES: COc1ncc(NC(=O)C(=O)N2C[C@@H](C)CC[C@@H]2c2ccc3sc([C@]45CCC(C4)N(C)C5)nc3c2)cc1C(N)=O ; COc1ncc(NC(=O)C(=O)N2C[C@@H](C)CC[C@@H]2c2ccc3sc([C@@]45CCC(C4)N(C)C5)nc3c2)cc1C(N)=O ; CCc1cc(NC(=O)C(=O)N2C[C@@H](C)CC[C@@H]2c2ccc3nn(CCN(C)C)cc3c2)cnc1N] 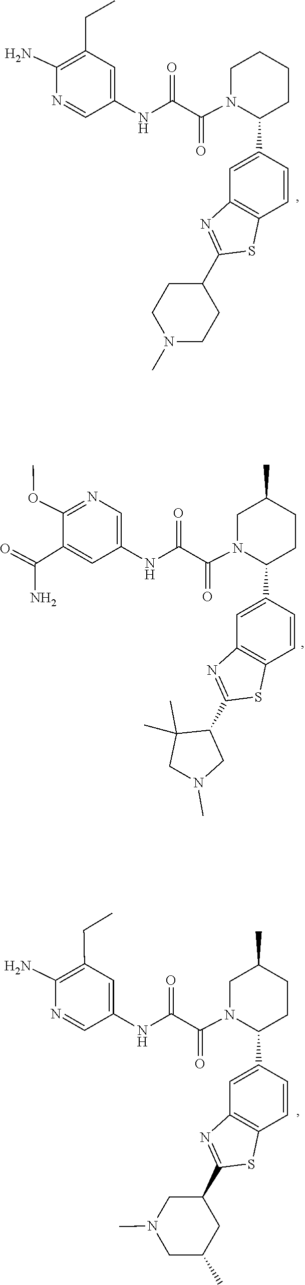 [SMILES: CCc1cc(NC(=O)C(=O)N2CCCC[C@@H]2c2ccc3sc(C4CCN(C)CC4)nc3c2)cnc1N ; COc1ncc(NC(=O)C(=O)N2C[C@@H](C)CC[C@@H]2c2ccc3sc([C@@H]4CN(C)CC4(C)C)nc3c2)cc1C(N)=O ; CCc1cc(NC(=O)C(=O)N2C[C@@H](C)CC[C@@H]2c2ccc3sc([C@H]4C[C@H](C)CN(C)C4)nc3c2)cnc1N] 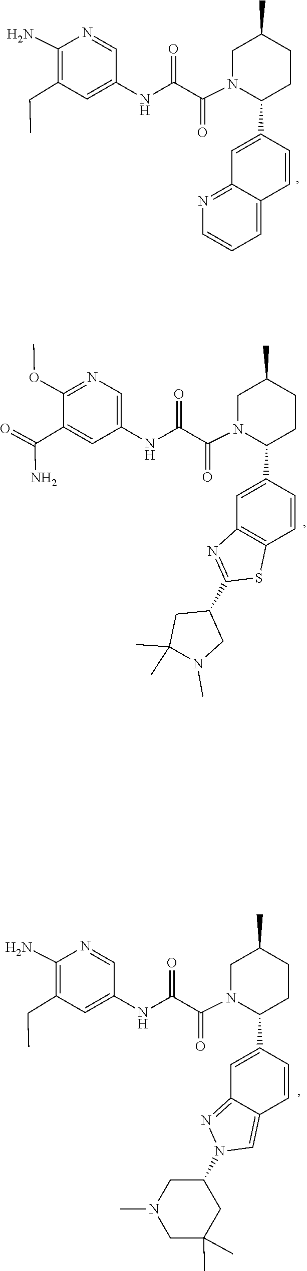 [SMILES: CCc1cc(NC(=O)C(=O)N2C[C@@H](C)CC[C@@H]2c2ccc3cccnc3c2)cnc1N ; COc1ncc(NC(=O)C(=O)N2C[C@@H](C)CC[C@@H]2c2ccc3sc([C@@H]4CN(C)C(C)(C)C4)nc3c2)cc1C(N)=O ; CCc1cc(NC(=O)C(=O)N2C[C@@H](C)CC[C@@H]2c2ccc3cn([C@H]4CN(C)CC(C)(C)C4)nc3c2)cnc1N] 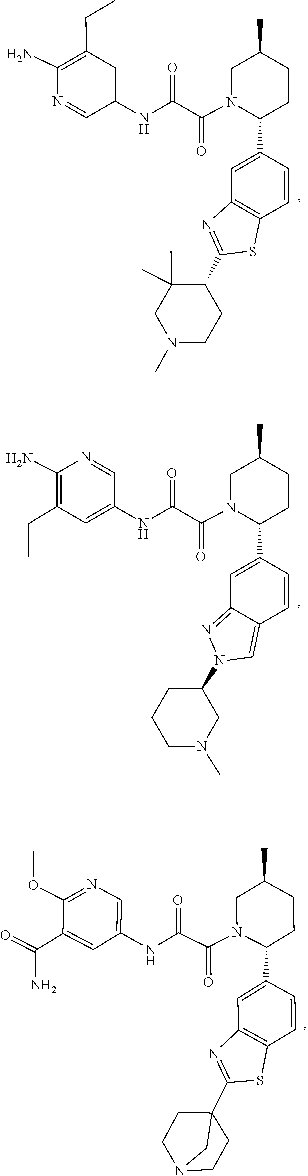 [SMILES: CCC1=C(N)N=CC(NC(=O)C(=O)N2C[C@@H](C)CC[C@@H]2c2ccc3sc([C@@H]4CCN(C)CC4(C)C)nc3c2)C1 ; CCc1cc(NC(=O)C(=O)N2C[C@@H](C)CC[C@@H]2c2ccc3cn([C@@H]4CCCN(C)C4)nc3c2)cnc1N ; COc1ncc(NC(=O)C(=O)N2C[C@@H](C)CC[C@@H]2c2ccc3sc(C45CCN(CC4)C5)nc3c2)cc1C(N)=O] 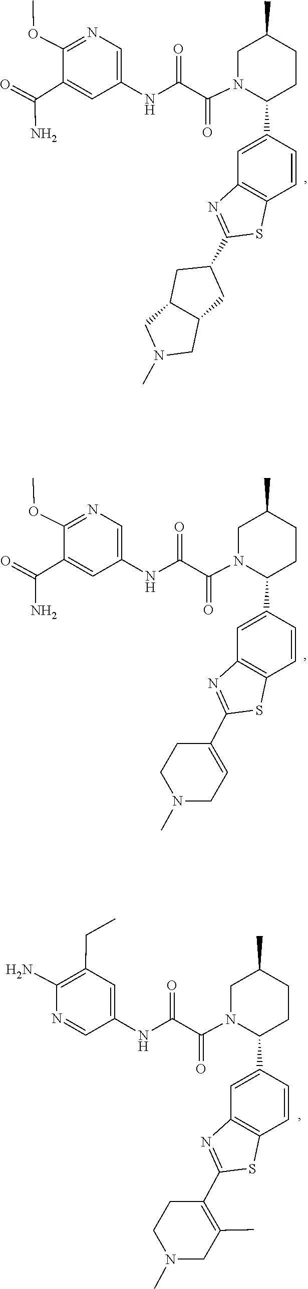 [SMILES: COc1ncc(NC(=O)C(=O)N2C[C@@H](C)CC[C@@H]2c2ccc3sc([C@@H]4C[C@@H]5CN(C)C[C@@H]5C4)nc3c2)cc1C(N)=O ; COc1ncc(NC(=O)C(=O)N2C[C@@H](C)CC[C@@H]2c2ccc3sc(C4=CCN(C)CC4)nc3c2)cc1C(N)=O ; CCc1cc(NC(=O)C(=O)N2C[C@@H](C)CC[C@@H]2c2ccc3sc(C4=C(C)CN(C)CC4)nc3c2)cnc1N] 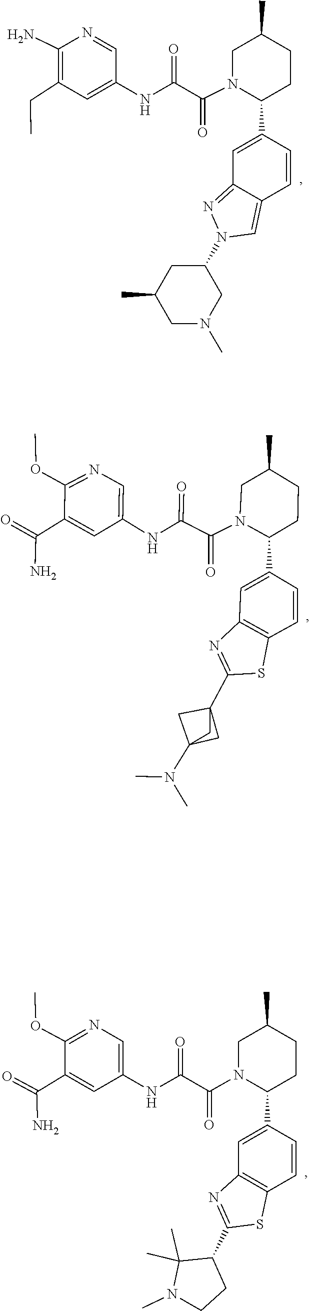 [SMILES: CCc1cc(NC(=O)C(=O)N2C[C@@H](C)CC[C@@H]2c2ccc3cn([C@H]4C[C@H](C)CN(C)C4)nc3c2)cnc1N ; COc1ncc(NC(=O)C(=O)N2C[C@@H](C)CC[C@@H]2c2ccc3sc(C45CC(N(C)C)(C4)C5)nc3c2)cc1C(N)=O ; COc1ncc(NC(=O)C(=O)N2C[C@@H](C)CC[C@@H]2c2ccc3sc([C@@H]4CCN(C)C4(C)C)nc3c2)cc1C(N)=O] 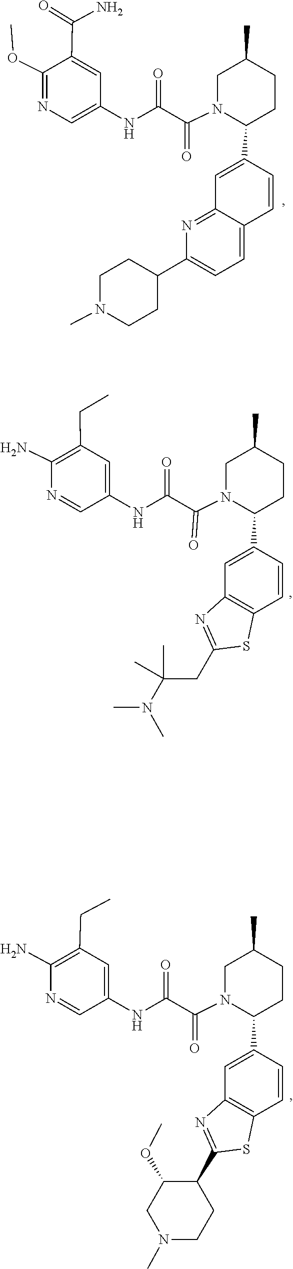 [SMILES: COc1ncc(NC(=O)C(=O)N2C[C@@H](C)CC[C@@H]2c2ccc3ccc(C4CCN(C)CC4)nc3c2)cc1C(N)=O ; CCc1cc(NC(=O)C(=O)N2C[C@@H](C)CC[C@@H]2c2ccc3sc(CC(C)(C)N(C)C)nc3c2)cnc1N ; CCc1cc(NC(=O)C(=O)N2C[C@@H](C)CC[C@@H]2c2ccc3sc([C@H]4CCN(C)C[C@@H]4OC)nc3c2)cnc1N] 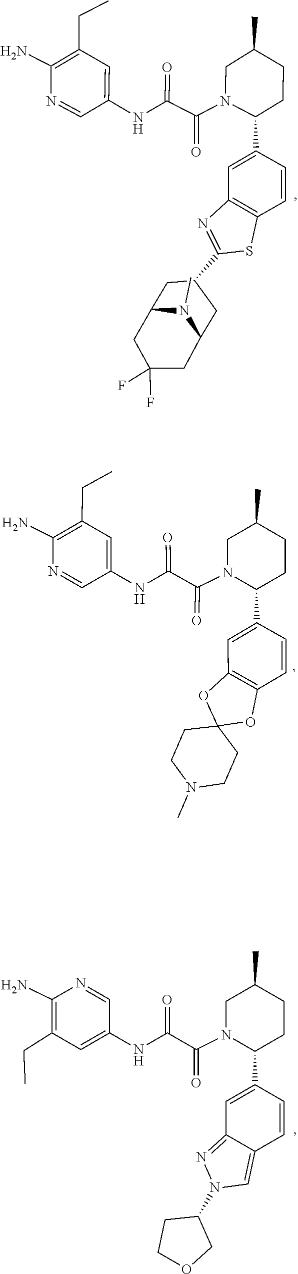 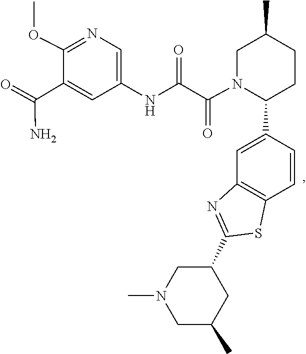 [SMILES: COc1ncc(NC(=O)C(=O)N2C[C@@H](C)CC[C@@H]2c2ccc3sc([C@@H]4C[C@@H](C)CN(C)C4)nc3c2)cc1C(N)=O] 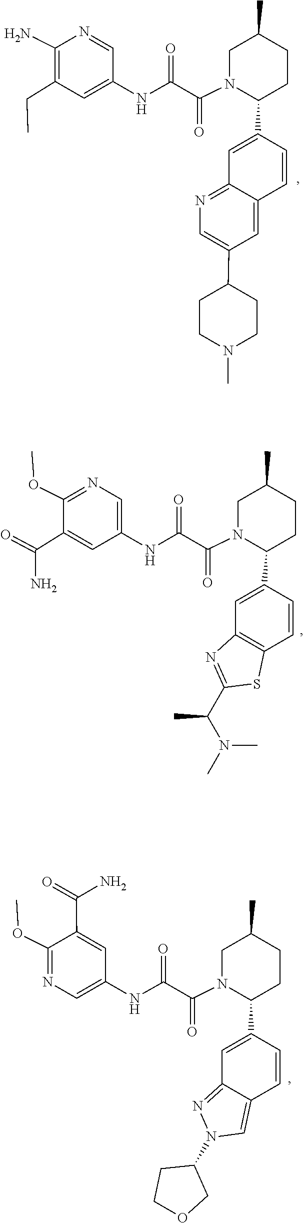 [SMILES: CCc1cc(NC(=O)C(=O)N2C[C@@H](C)CC[C@@H]2c2ccc3cc(C4CCN(C)CC4)cnc3c2)cnc1N ; COc1ncc(NC(=O)C(=O)N2C[C@@H](C)CC[C@@H]2c2ccc3sc([C@H](C)N(C)C)nc3c2)cc1C(N)=O ; COc1ncc(NC(=O)C(=O)N2C[C@@H](C)CC[C@@H]2c2ccc3cn([C@H]4CCOC4)nc3c2)cc1C(N)=O] 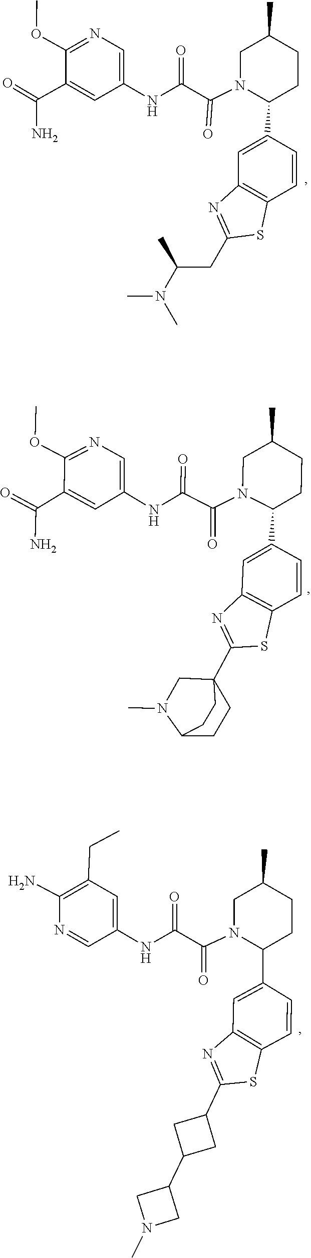 [SMILES: COc1ncc(NC(=O)C(=O)N2C[C@@H](C)CC[C@@H]2c2ccc3sc(C[C@H](C)N(C)C)nc3c2)cc1C(N)=O ; COc1ncc(NC(=O)C(=O)N2C[C@@H](C)CC[C@@H]2c2ccc3sc(C45CCC(CC4)N(C)C5)nc3c2)cc1C(N)=O ; CCc1cc(NC(=O)C(=O)N2C[C@@H](C)CCC2c2ccc3sc(C4CC(C5CN(C)C5)C4)nc3c2)cnc1N] 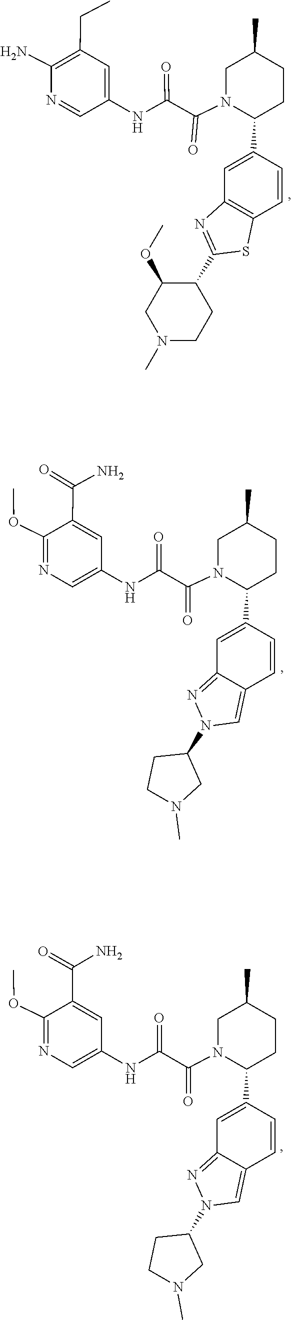 [SMILES: CCc1cc(NC(=O)C(=O)N2C[C@@H](C)CC[C@@H]2c2ccc3sc([C@@H]4CCN(C)C[C@H]4OC)nc3c2)cnc1N ; COc1ncc(NC(=O)C(=O)N2C[C@@H](C)CC[C@@H]2c2ccc3cn([C@@H]4CCN(C)C4)nc3c2)cc1C(N)=O ; COc1ncc(NC(=O)C(=O)N2C[C@@H](C)CC[C@@H]2c2ccc3cn([C@H]4CCN(C)C4)nc3c2)cc1C(N)=O] 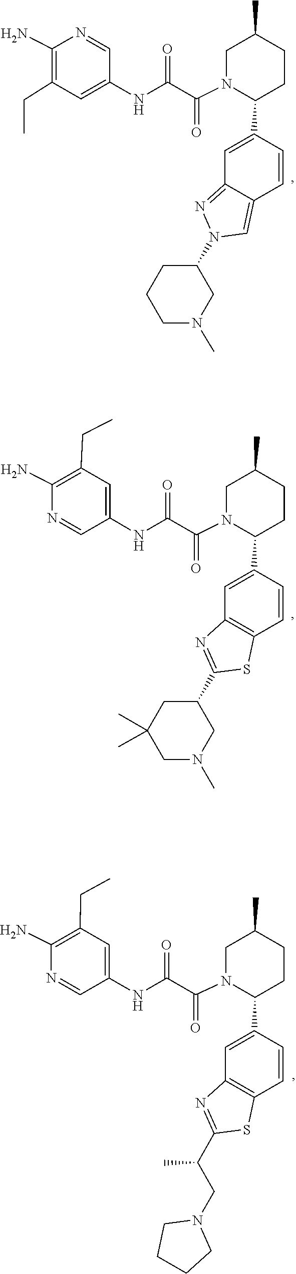 [SMILES: CCc1cc(NC(=O)C(=O)N2C[C@@H](C)CC[C@@H]2c2ccc3cn([C@H]4CCCN(C)C4)nc3c2)cnc1N ; CCc1cc(NC(=O)C(=O)N2C[C@@H](C)CC[C@@H]2c2ccc3sc([C@@H]4CN(C)CC(C)(C)C4)nc3c2)cnc1N ; CCc1cc(NC(=O)C(=O)N2C[C@@H](C)CC[C@@H]2c2ccc3sc([C@@H](C)CN4CCCC4)nc3c2)cnc1N] 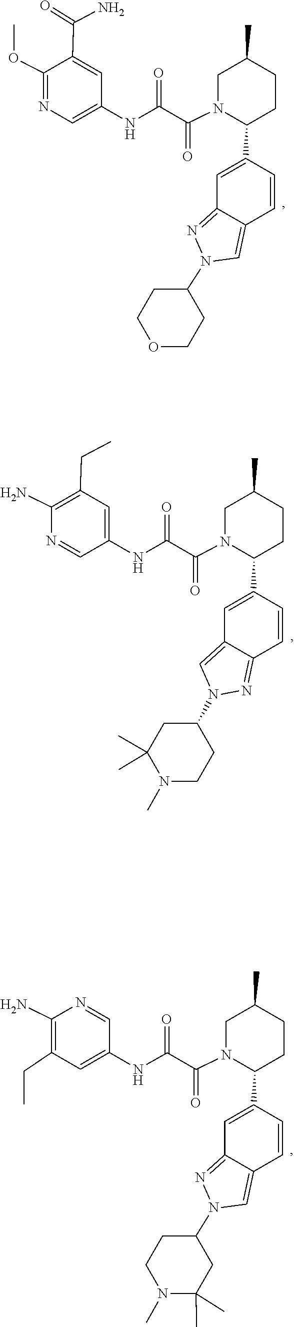 [SMILES: COc1ncc(NC(=O)C(=O)N2C[C@@H](C)CC[C@@H]2c2ccc3cn(C4CCOCC4)nc3c2)cc1C(N)=O ; CCc1cc(NC(=O)C(=O)N2C[C@@H](C)CC[C@@H]2c2ccc3nn([C@@H]4CCN(C)C(C)(C)C4)cc3c2)cnc1N ; CCc1cc(NC(=O)C(=O)N2C[C@@H](C)CC[C@@H]2c2ccc3cn(C4CCN(C)C(C)(C)C4)nc3c2)cnc1N] 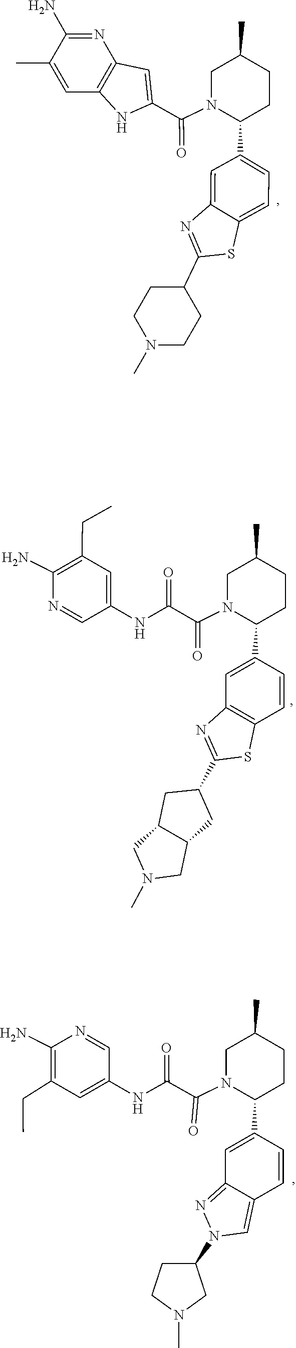 [SMILES: Cc1cc2[nH]c(C(=O)N3C[C@@H](C)CC[C@@H]3c3ccc4sc(C5CCN(C)CC5)nc4c3)cc2nc1N ; CCc1cc(NC(=O)C(=O)N2C[C@@H](C)CC[C@@H]2c2ccc3sc([C@@H]4C[C@@H]5CN(C)C[C@@H]5C4)nc3c2)cnc1N ; CCc1cc(NC(=O)C(=O)N2C[C@@H](C)CC[C@@H]2c2ccc3cn([C@@H]4CCN(C)C4)nc3c2)cnc1N] 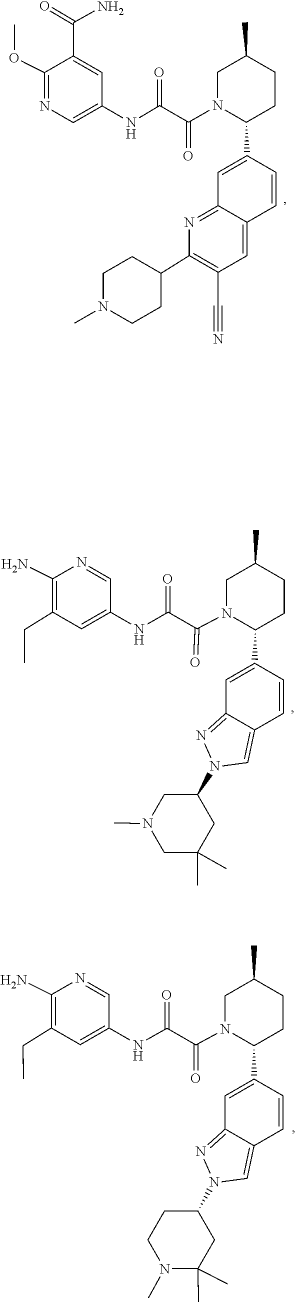 [SMILES: COc1ncc(NC(=O)C(=O)N2C[C@@H](C)CC[C@@H]2c2ccc3cc(C#N)c(C4CCN(C)CC4)nc3c2)cc1C(N)=O ; CCc1cc(NC(=O)C(=O)N2C[C@@H](C)CC[C@@H]2c2ccc3cn([C@@H]4CN(C)CC(C)(C)C4)nc3c2)cnc1N ; CCc1cc(NC(=O)C(=O)N2C[C@@H](C)CC[C@@H]2c2ccc3cn([C@H]4CCN(C)C(C)(C)C4)nc3c2)cnc1N] 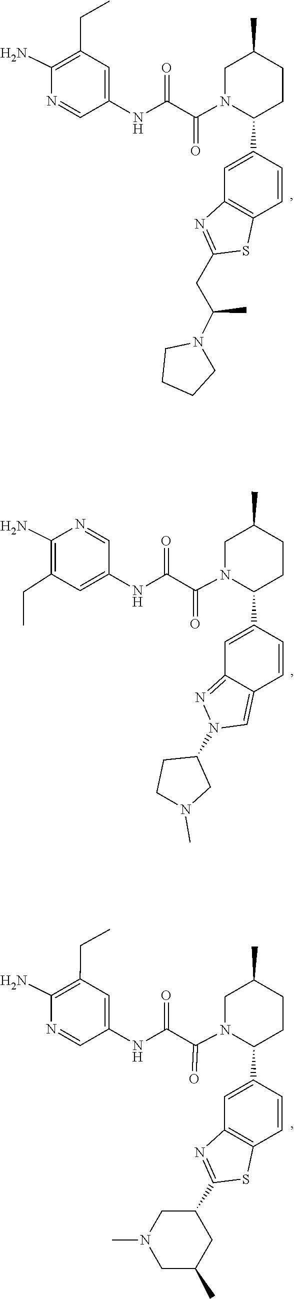 [SMILES: CCc1cc(NC(=O)C(=O)N2C[C@@H](C)CC[C@@H]2c2ccc3sc(C[C@@H](C)N4CCCC4)nc3c2)cnc1N ; CCc1cc(NC(=O)C(=O)N2C[C@@H](C)CC[C@@H]2c2ccc3cn([C@H]4CCN(C)C4)nc3c2)cnc1N ; CCc1cc(NC(=O)C(=O)N2C[C@@H](C)CC[C@@H]2c2ccc3sc([C@@H]4C[C@@H](C)CN(C)C4)nc3c2)cnc1N] 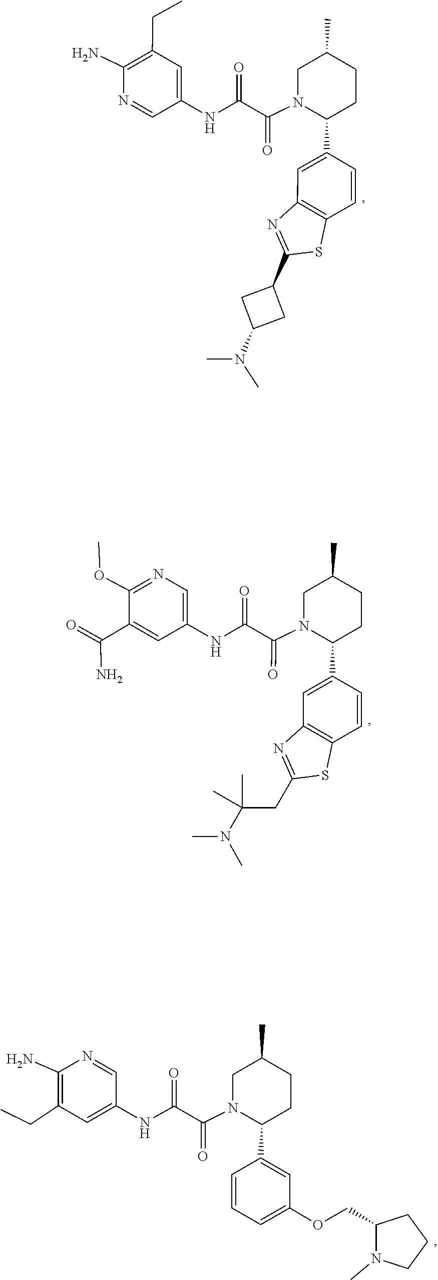 [SMILES: CCc1cc(NC(=O)C(=O)N2C[C@H](C)CC[C@@H]2c2ccc3sc([C@H]4C[C@H](N(C)C)C4)nc3c2)cnc1N ; COc1ncc(NC(=O)C(=O)N2C[C@@H](C)CC[C@@H]2c2ccc3sc(CC(C)(C)N(C)C)nc3c2)cc1C(N)=O ; CCc1cc(NC(=O)C(=O)N2C[C@@H](C)CC[C@@H]2c2cccc(OC[C@@H]3CCCN3C)c2)cnc1N] 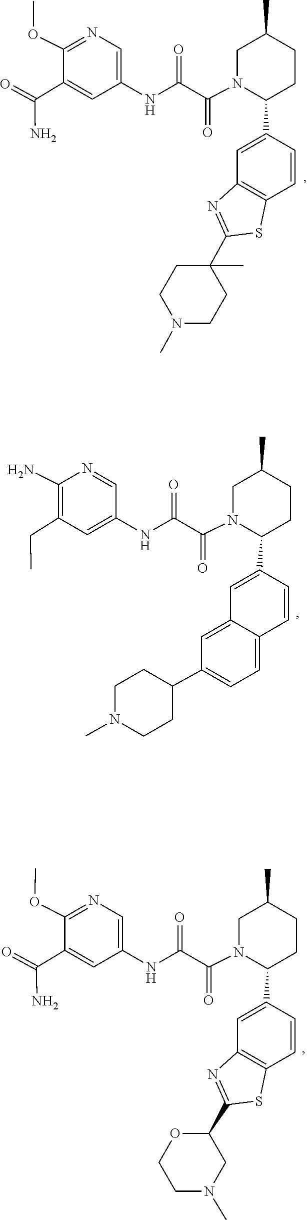 [SMILES: COc1ncc(NC(=O)C(=O)N2C[C@@H](C)CC[C@@H]2c2ccc3sc(C4(C)CCN(C)CC4)nc3c2)cc1C(N)=O ; CCc1cc(NC(=O)C(=O)N2C[C@@H](C)CC[C@@H]2c2ccc3ccc(C4CCN(C)CC4)cc3c2)cnc1N ; COc1ncc(NC(=O)C(=O)N2C[C@@H](C)CC[C@@H]2c2ccc3sc([C@H]4CN(C)CCO4)nc3c2)cc1C(N)=O] 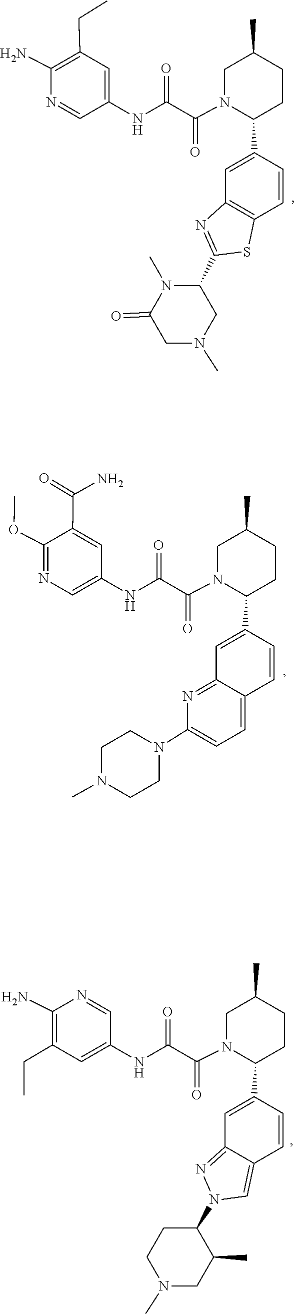 [SMILES: CCc1cc(NC(=O)C(=O)N2C[C@@H](C)CC[C@@H]2c2ccc3sc([C@@H]4CN(C)CC(=O)N4C)nc3c2)cnc1N ; COc1ncc(NC(=O)C(=O)N2C[C@@H](C)CC[C@@H]2c2ccc3ccc(N4CCN(C)CC4)nc3c2)cc1C(N)=O ; CCc1cc(NC(=O)C(=O)N2C[C@@H](C)CC[C@@H]2c2ccc3cn([C@@H]4CCN(C)C[C@@H]4C)nc3c2)cnc1N] 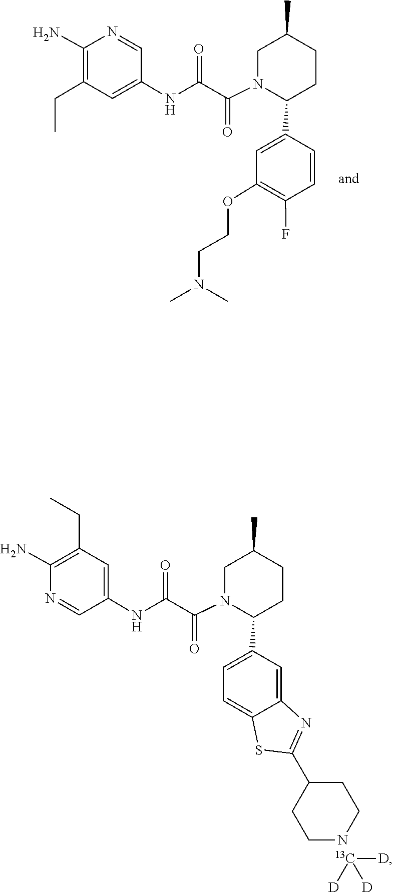
or a pharmaceutically acceptable salt thereof.

2. A pharmaceutical composition comprising a compound of claim 1, or a pharmaceutically acceptable salt thereof, and a pharmaceutically acceptable carrier.

3. The pharmaceutical composition of claim 2, further comprising a second therapeutic agent.

4. A method of treating an MTAP-deficient and/or an MTA-accumulating disease in a subject in need thereof by administering to the subject an effective amount (e.g., a therapeutically effective amount) of a compound of claim 1, or a pharmaceutically acceptable salt thereof, or a pharmaceutically acceptable composition of claim 2.

5. The method of claim 4 wherein the compound, or a pharmaceutically acceptable salt thereof, or composition is administered in combination with a second therapeutic agent.

6. A method of treating an MTAP-deficient and/or an MTA-accumulating disease in a subject in need thereof by administering to the subject an effective amount (e.g., a therapeutically effective amount) of a pharmaceutically acceptable composition of claim 3.

7. The method of any one of claims 4 to 6 wherein the disease is a proliferating disease.

8. The method of claim 7 wherein the disease is an MTAP-deficient and/or MTA-accumulating cancer.

9. The method of claim 8 wherein the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

10. A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering a therapeutically effective amount of a compound of claim 1, or a pharmaceutically acceptable salt thereof, or composition of claim 2 or 3 to the subject identified in step b).
